(19) United States
(12) Patent Application Publication
(10) Pub. No.: US 2026/0108613 A1
(43) Pub. Date: Apr. 23, 2026

(54) Compounds and Compositions as VHL Ligands and STAT3 Degraders Uses Thereof

(71) Applicant: REGENTS OF THE UNIVERSITY OF MICHIGAN, Ann Arbor, MI (US)

(72) Inventors: Shaomeng Wang, Superior Township, MI (US); Haibin Zhou, Ann Arbor, MI (US); Dimin Wu, Ann Arbor, MI (US); Ranjan Kumar Acharyya, Ann Arbor, MI (US); Longchuan Bai, Ann Arbor, MI (US); Donna McEachern, Grass Lake, MI (US); Hoda Metwally, Ypsilanti, MI (US); Mi Wang, Ann Arbor, MI (US); Paul Kirchhoff, Saline, MI (US); Jelena Tosovic, Ann Arbor, MI (US)

(21) Appl. No.: 19/426,229

(22) Filed: Dec. 19, 2025

Related U.S. Application Data
(63) Continuation-in-part of application No. PCT/US24/34664, filed on Jun. 20, 2024.
(60) Provisional application No. 63/521,994, filed on Jun. 20, 2023.
(60) Provisional application No. 63/522,002, filed on Jun. 20, 2023.
(60) Provisional application No. 63/598,423, filed on Nov. 13, 2023.
(60) Provisional application No. 63/598,434, filed on Nov. 13, 2023.

(51) Int. Cl. A61K 47/54; C07D 417/14; C07D 487/04; C07D 498/04

(52) U.S. Cl. CPC A61K 47/548; C07D 417/14; C07D 487/04; C07D 498/04

(57) ABSTRACT

Described herein are compounds or conjugates of Formula I or II and their pharmaceutically acceptable salts, solvates, or stereoisomers, as well as their uses (e.g., as VHL-binding agents or bifunctional degraders for degrading certain proteins (e.g., STAT3)).

### RELATED APPLICATIONS

This application is a continuation-in-part of International Application No. PCT/US2024/034664, filed Jun. 20, 2024, which claims the benefit of and priority to U.S. Provisional Application No. 63/521,994, filed Jun. 20, 2023; U.S. Provisional Application No. 63/598,423, filed Nov. 13, 2023; U.S. Provisional Application No. 63/522,002, filed Jun. 20, 2023; and U.S. Provisional Application No. 63/598,434, filed Nov. 13, 2023; the contents of each of which are incorporated herein by reference in their entireties.

### BACKGROUND

E3 ubiquitin ligases confer substrate specificity for ubiquitination and are more attractive therapeutic targets than general proteasome inhibitors due to their specificity for certain protein substrates. Although the development of ligands of E3 ligase has proven challenging, in part due to the fact that they must disrupt protein-protein interactions, recent developments have provided specific ligands which bind to these ligases. However, the field remains underdeveloped.

One E3 ligase with exciting therapeutic potential is the von Hippel-Lindau (VHL) tumor suppressor, the substrate recognition subunit of the E3 ligase complex VCB, which also consists of elongins B and C, Cul2 and Rbxl. The primary substrate of VHL is Hypoxia Inducible Factor la (HIF-la), a transcription factor that upregulates genes such as the pro-angiogenic growth factor VEGF and the red blood cell inducing cytokine erythropoietin in response to low oxygen levels. While HIF-la is constitutively expressed, its intracellular levels are kept very low under normoxic conditions via its hydroxylation by prolyl hydroxylase domain (PHD) proteins and subsequent VHL-mediated ubiquitination.

The crystal structure of VHL with ligands has been obtained, confirming that a small compound can mimic the binding mode of the transcription factor HIF-la, the major substrate of VHL. Using rational design, the first small molecule ligands of Von Hippel Lindau (VHL) the substrate recognition subunit of the E3 ligase VCB (an important target in cancer, chronic anemia and ischemia) were generated. However, an ongoing need exists in the art for effective small molecule therapeutics across disease indications.

The signal transducer and activator of transcription (STAT) proteins play important roles in biological processes. For example, the abnormal activation of STAT signaling pathways is implicated in cancer, autoimmune diseases, rheumatoid arthritis, asthma, diabetes, and other human diseases. See, e.g., Miklossy et al., Nat Rev Drug Discov 12:611-629 (2013).

The STAT protein family is composed of seven members: STAT1, STAT2, STAT3, STAT4, STAT5A, STAT5B, and STAT6. Structurally, they share five domains: an amino-terminal domain, a coiled-coil domain, a DNA-binding domain, an SH2 domain, and a carboxy-terminal transactivation domain. The transactivation domain contains one or two amino acid residues that are crucial for the activity of the STAT protein. In particular, phosphorylation of a particular tyrosine residue promotes dimerization, whereas phosphorylation of a particular serine residue enhances transcriptional activation.

STAT proteins promote fundamental cellular processes, including cell growth and differentiation, development, apoptosis, immune responses, and inflammation. In particular, STAT3 function may be abnormal in the context of cancer, and this abnormality represents an underlying mechanism of STAT3 for promoting malignant transformation and progression. Constitutively active STAT3 is detected in numerous malignancies, including breast, melanoma, prostate, head and neck squamous cell carcinoma (HNSCC), multiple myeloma, pancreatic, ovarian, and brain tumors. Aberrant STAT3 signaling promotes tumorigenesis and tumor progression partly through dysregulating the expression of critical genes that control cell growth and survival, angiogenesis, migration, invasion, or metastasis. These genes include those that encode p21^{WAF1/CIP2}, cyclin D1, MYC, BCL-X, BCL-2, vascular endothelial growth factor (VEGF), matrix metalloproteinase 1 (MMP1), MMP7 and MMP9, and survivin. STAT3 may also play a role in the suppression of tumor immune surveillance. Consequently, the genetic and pharmacological modulation of persistently active STAT3 was shown to control the tumor phenotype and to lead to tumor regression in vivo.

There exists a need in the art for STAT3 inhibitors and STAT3 degraders having physical and pharmacological properties that allow them to be used in therapeutic applications for treating disease.

### SUMMARY

In certain aspects, the present disclosure provides compounds or conjugates of Formula I:

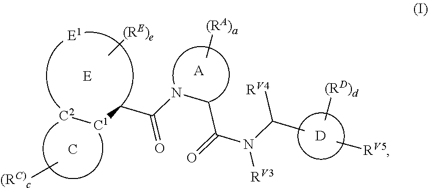

and pharmaceutically acceptable salts, solvates, or stereoisomers thereof, wherein each of the variables in Formulae I is described, embodied, and exemplified herein.

In certain aspects, the present disclosure provides pharmaceutical compositions comprising a compound disclosed herein, and a pharmaceutically acceptable excipient.

In certain aspects, the present disclosure provides methods of binding VHL E3 ubiquitin ligase protein complex in a subject or biological sample comprising administering the compound disclosed herein or contacting the biological sample with the compound disclosed herein.

In certain aspects, the present disclosure provides uses of the compound disclosed herein in the manufacture of a medicament for binding VHL E3 ubiquitin ligase protein complex in a subject or biological sample.

In certain aspects, the present disclosure provides compounds disclosed herein for use in binding VHL E3 ubiquitin ligase protein complex in a subject or biological sample.

In certain aspects, the present disclosure provides methods of degrading a protein in a subject or biological sample comprising administering a compound or conjugate disclosed herein to the subject or contacting the biological sample with the compound disclosed herein.

In certain aspects, the present disclosure provides uses of a compound or conjugate disclosed herein in the manufacture of a medicament for degrading a protein in a subject or biological sample.

In certain aspects, the present disclosure provides compounds or conjugates disclosed herein for use in degrading a protein in a subject or biological sample.

In certain aspects, the present disclosure provides methods of treating or preventing a disease or disorder in a subject in need thereof, comprising administering to the subject a compound or conjugate disclosed herein.

In certain aspects, the present disclosure provides methods of treating a disease or disorder in a subject in need thereof, comprising administering to the subject a compound or conjugate disclosed herein.

In certain aspects, the present disclosure provides uses of a compound or conjugate disclosed herein in the manufacture of a medicament for treating or preventing a disease or disorder in a subject in need thereof.

In certain aspects, the present disclosure provides uses of a compound or conjugate disclosed herein in the manufacture of a medicament for treating a disease or disorder in a subject in need thereof.

In certain aspects, the present disclosure provides compounds or conjugates disclosed herein for use in treating or preventing a disease or disorder in a subject in need thereof.

In certain aspects, the present disclosure provides compounds or conjugates disclosed herein for use in treating a disease or disorder in a subject in need thereof.

In certain aspects, the present disclosure provides conjugates of Formula II:

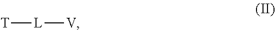

and pharmaceutically acceptable salts, solvates, or stereoisomers thereof, wherein:
V is of Formula II-1

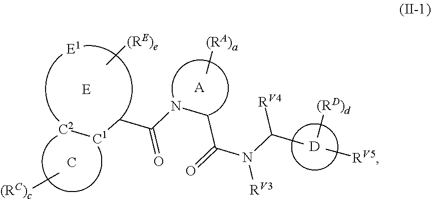

### L is of Formula II-2

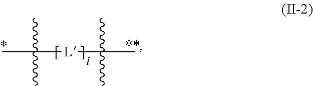

and
T is of Formula II-3

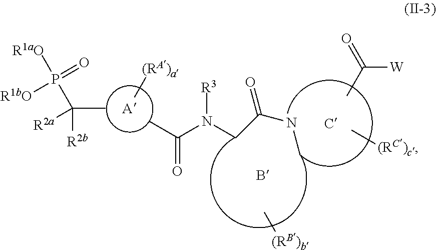

wherein each of the variables in Formulae II, II-1, 11-2, and II-3, is described, embodied, and exemplified herein.

In certain aspects, the present disclosure provides pharmaceutical compositions comprising a conjugate disclosed herein, and a pharmaceutically acceptable excipient.

In certain aspects, the present disclosure provides methods of degrading a protein (e.g., STAT3) in a subject or biological sample comprising administering a conjugate disclosed herein to the subject or contacting the biological sample with the conjugates disclosed herein.

In certain aspects, the present disclosure provides uses of a conjugate disclosed herein in the manufacture of a medicament for degrading a protein (e.g., STAT3) in a subject or biological sample.

In certain aspects, the present disclosure provides conjugates disclosed herein for use in degrading a protein (e.g., STAT3) in a subject or biological sample.

In certain aspects, the present disclosure provides methods of reducing a protein (e.g., STAT3) in a subject or biological sample comprising administering a conjugate disclosed herein to the subject or contacting the biological sample with the conjugate disclosed herein.

In certain aspects, the present disclosure provides uses of a conjugate disclosed herein in the manufacture of a medicament for reducing a protein (e.g., STAT3) in a subject or biological sample.

In certain aspects, the present disclosure provides conjugates disclosed herein for use in reducing a protein (e.g., STAT3) in a subject or biological sample.

In certain aspects, the present disclosure provides methods of treating or preventing a disease or disorder (e.g., a STAT3-mediated disease or disorder) in a subject in need thereof, comprising administering to the subject a conjugate disclosed herein (e.g., in a therapeutically effective amount).

In certain aspects, the present disclosure provides methods of treating a disease or disorder (e.g., a STAT3-mediated disease or disorder) in a subject in need thereof, comprising administering to the subject a conjugate disclosed herein (e.g., in a therapeutically effective amount).

In certain aspects, the present disclosure provides uses of a conjugate disclosed herein in the manufacture of a medicament for treating or preventing a disease or disorder (e.g., a STAT3-mediated disease or disorder) in a subject in need thereof.

In certain aspects, the present disclosure provides uses of a conjugate disclosed herein in the manufacture of a medicament for treating a disease or disorder (e.g., a STAT3-mediated disease or disorder) in a subject in need thereof.

In certain aspects, the present disclosure provides conjugates disclosed herein for use in treating or preventing a disease or disorder (e.g., a STAT3-mediated disease or disorder) in a subject in need thereof.

In certain aspects, the present disclosure provides conjugates disclosed herein for use in treating a disease or disorder (e.g., a STAT3-mediated disease or disorder) in a subject in need thereof.

### DESCRIPTION OF THE DRAWINGS

FIG. 1 is a graph showing antitumor activity (SUP-M2 tumor growth) of STAT3 degrader B1 in SUP-M2 lymphoma xenograft models in SCID mice.

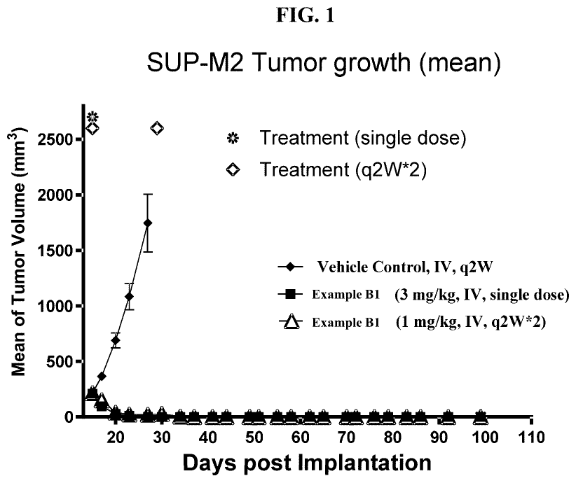

FIG. 2 is a graph showing antitumor activity (SUP-M2 body weight) of STAT3 degrader B1 in SUP-M2 lymphoma xenograft models in SCID mice.

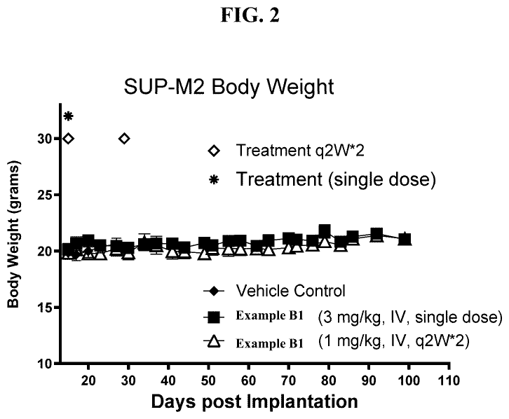

FIG. 3 is a graph showing antitumor activity (SU-DHL-1 tumor growth) of STAT3 degrader B3 in SU-DHL-1 lymphoma xenograft models in SCID mice.

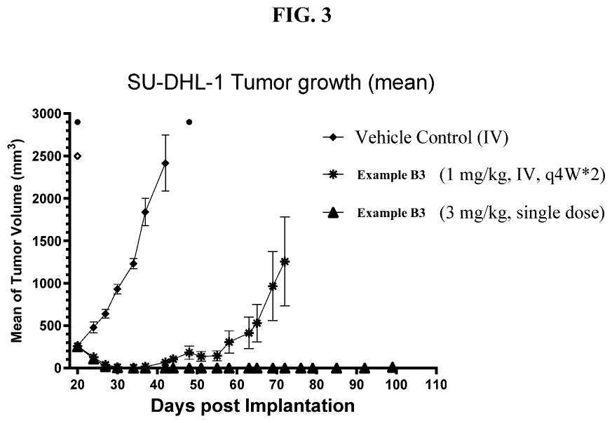

FIG. 4 is a graph showing antitumor activity (SU-DHL-1 body weight) of STAT3 degrader B3 in SU-DHL-1 lymphoma xenograft models in SCID mice.

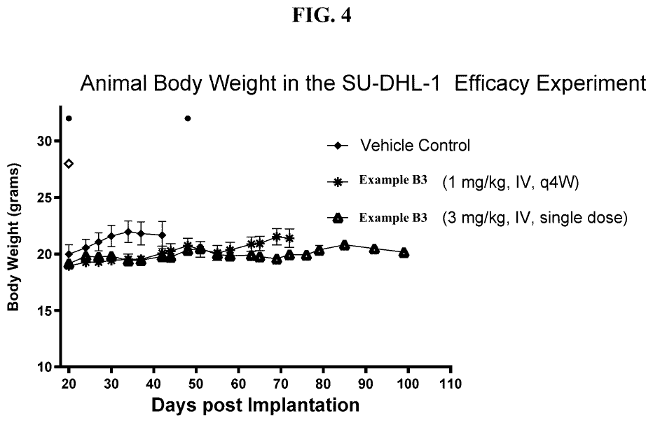

FIG. 5 is a graph showing antitumor activity (SUP-M2 tumor growth) of STAT3 degrader B3 in SUP-M2 lymphoma xenograft models in SCID mice.

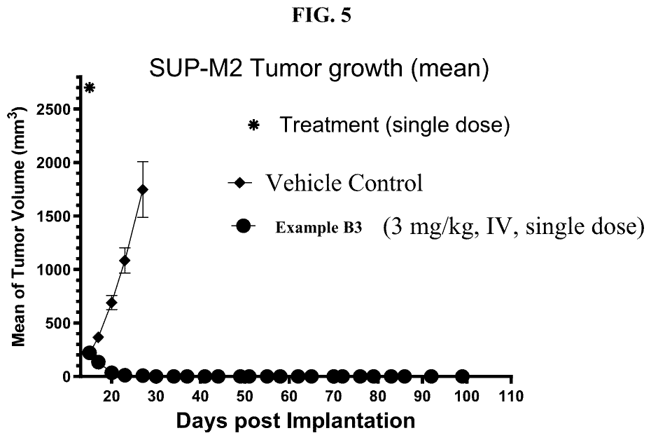

FIG. 6 is a graph showing antitumor activity (SUP-M2 body weight) of STAT3 degrader B3 in SUP-M2 lymphoma xenograft models in SCID mice.

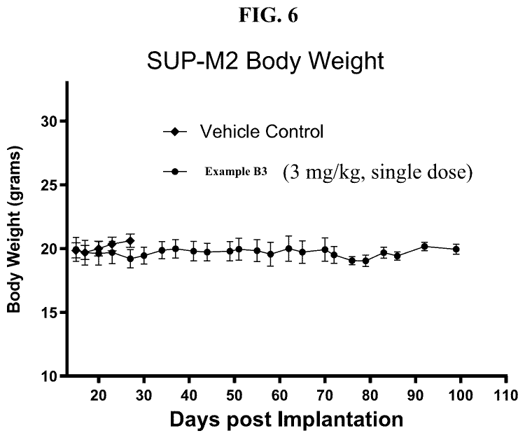

FIG. 7 is a graph showing antitumor activity (MDA-MB-468 tumor growth) of STAT3 degrader B3 in the MDA-MB-468 triple-negative breast cancer model.

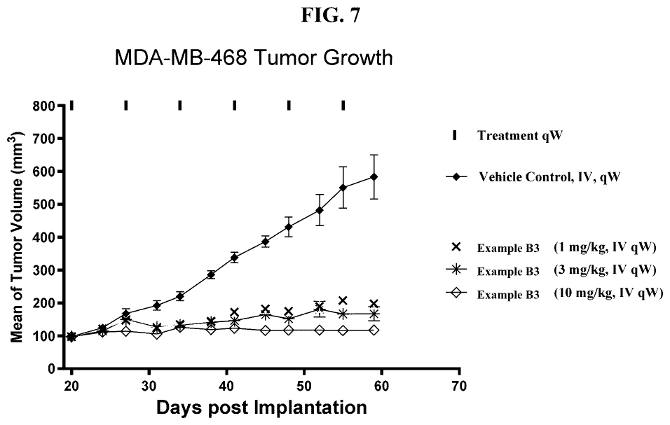

### DETAILED DESCRIPTION

The present disclosure relates to compounds that show VHL E3 ubiquitin ligase protein complex-binding activity, bifunctional degraders comprising a VHL E3 ubiquitin ligase protein complex-binding moeity, and pharmaceutical compositions comprising such compounds or bifunctional degraders. The present disclosure further relates to methods of binding VHL E3 ubiquitin ligase protein complex in a subject or biological sample comprising administering a compound described herein to the subject or contacting the biological sample with a compound described herein. The present disclosure further relates to methods of degrading a protein in a subject or biological sample comprising administering a compound described herein to the subject or contacting the biological sample with a compound described herein. The present disclosure also relates to methods of treating or preventing a disease or disorder a subject in need thereof, comprising administering to the subject a compound described herein.

The present disclosure further relates to conjugates that may be useful as degraders for certain proteins (e.g., STAT3). The present disclosure also relates to methods of degrading certain proteins (e.g., STAT3) comprising contacting the STAT3 protein with a conjugate disclosed herein. The present disclosure also relates to methods of treating a STAT3-mediated disease or condition in a subject in need thereof by administering (e.g., in a therapeutically effective amount) a conjugate disclosed herein. The present disclosure further relates to methods of treating a STAT3-mediated disease or condition in a subject in need thereof, comprising administering (e.g., in a therapeutically effective amount) a pharmaceutical composition comprising an amount of a conjugate disclosed herein.

### Compounds of the Present Disclosure

### VHL Ligands

In certain aspects, the present disclosure provides compounds of Formula I:

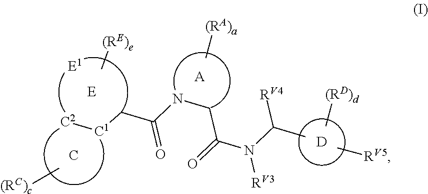

and pharmaceutically acceptable salts, solvates, or stereoisomers thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently amino-protecting group, oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is hydrogen, C₁₋₆ alkyl, C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound is a compound of Formula I-0 or I-0′

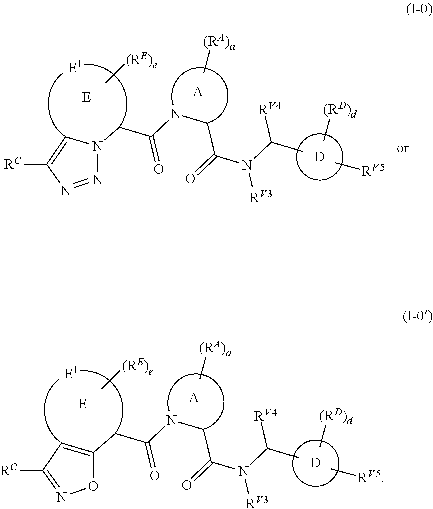

In certain embodiments, the compound is a compound of Formula I-1 or I-2

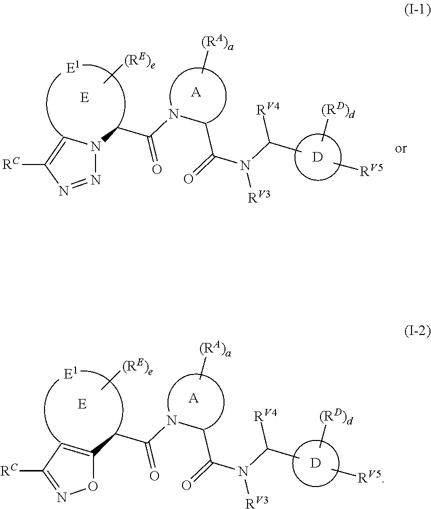

In certain embodiments, the compound is a compound of Formula I-1-i or I-2-i

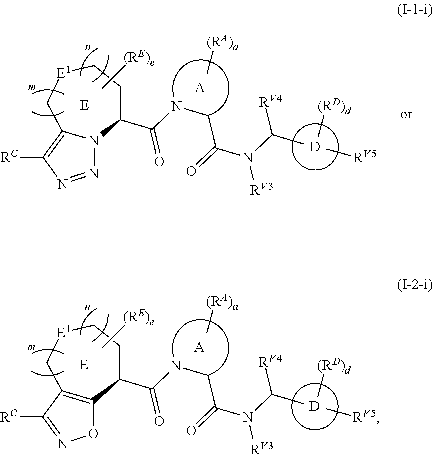

In certain embodiments, the compound is a compound of Formula I-1-ii or I-2-ii

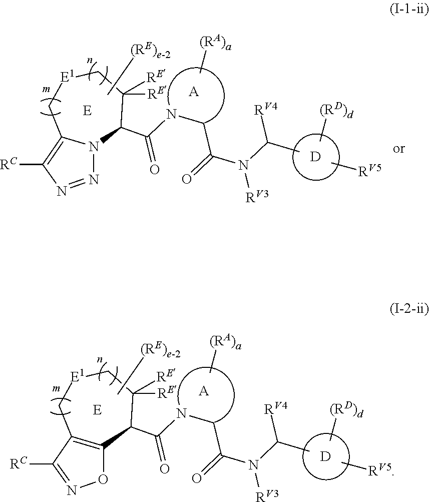

In certain embodiments, the compound is a compound of Formula I-1-ii or I-2-ii

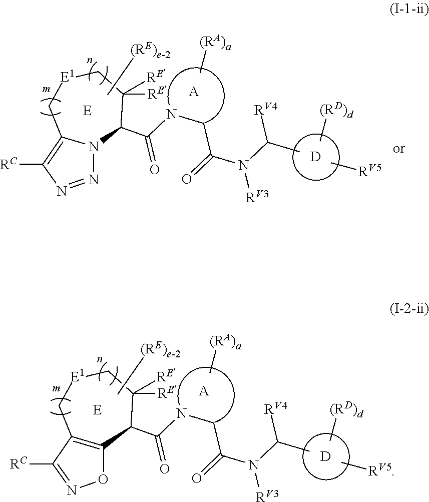

wherein each of R^{E′} is independently C₁₋₆ alkyl.

In certain embodiments, m is 1 or 2; and n is 0, 1, or 2.

In certain embodiments, m is 1. In certain embodiments, m is 2.

In certain embodiments, n is 0. In certain embodiments, n is 1. In certain embodiments, n is 2.

In certain embodiments, m is 1 and n is 1.

In certain embodiments, m is 2 and n is 1.

In certain embodiments, m is 1 and n is 0.

In certain embodiments, m is 1 and n is 2.

In certain embodiments, Ring E is C₅₋₁₂ carbocyclyl (e.g., cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 5- to 12-membered heterocyclyl (e.g., heterocyclyl comprising one or two 5- to 8-membered rings and 1-5 heteroatoms selected from N, O, and S), wherein the carbocyclyl or heterocyclyl is optionally substituted with one or more R^{u}.

In certain embodiments, each R^{E} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least two R^{E} are independently C₁₋₆ alkyl.

In certain embodiments, two geminal R^{E}, together with the carbon atom to which they are bonded, form C₃₋₆ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocycle (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, e is 0. In certain embodiments, e is 1. In certain embodiments, e is 2. In certain embodiments, e is 3. In certain embodiments, e is 4. In certain embodiments, e is 5. In certain embodiments, e is 6.

In certain embodiments, e is an integer selected from 2 to 6.

In certain embodiments, each R^{E′} is independently hydrogen, C₁₋₆ alkyl, or C₁₋₆ alkoxy (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), wherein at least one R^{E′} is not hydrogen.

In certain embodiments, each R^{E′} is independently C₁₋₆ alkyl. In certain embodiments, each R^{E′} is independently methyl, ethyl, or propyl. In certain embodiments, each R^{E′} is methyl.

In certain embodiments, two R^{E′}, together with the carbon atom to which they are bonded, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, R^{E} is R^{E′}.

In certain embodiments, E¹ is C(R^{E1′})₂, NR^{E1″}, O, or S. In certain embodiments, E¹ is C(R^{E1′})₂, NR^{E1′}, O, or S. In certain embodiments, E¹ is NR^{E1″}. In certain embodiments, E¹ is O. In certain embodiments, E¹ is S.

In certain embodiments, E¹ is C(R^{E1′})₂, NR^{E1″}, or O.

In certain embodiments, each R^{E1′} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkoxy, alkylamino, carbocyclyl, or heterocyclyl is optionally substituted with one or more R^{u}.

In certain embodiments, each R^{E1′} is independently hydrogen or C₁₋₆ alkyl.

In certain embodiments, R^{E1″} is an amino-protecting group (e.g., carbanates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)), hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u}.

In certain embodiments, R^{E1″} is amino-protecting group, hydrogen, C₁₋₆ alkyl, or —C(═O)R^{a}.

In certain embodiments, Ring A is 5- to 7-membered heterocycle. In certain embodiments, Ring A is 5-membered heterocycle. In certain embodiments, Ring A is 6-membered heterocycle. In certain embodiments, Ring A is 7-membered heterocycle.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH-2, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least one R^{A} is —OH.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 8, as valency permits. In certain embodiments, a is 9, as valency permits. In certain embodiments, a is 10, as valency permits.

In certain embodiments, a is 1 or 2, and at least one of R^{A} is —OH.

In certain embodiments,

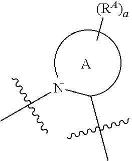

is

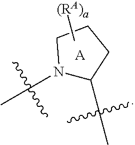

In certain embodiments,

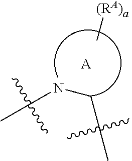

is

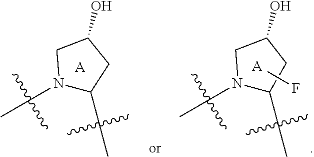

In certain embodiments, Ring C is C₆ aryl (i.e., phenyl) or 5- to 6-membered heteroaryl (e.g., heteroaryl comprising one 5- or 6-membered ring and 1-4 heteroatoms selected from N, O, and S).

In certain embodiments, Ring C is C₆ aryl (i.e., phenyl). In certain embodiments, Ring C is 5-membered heteroaryl. In certain embodiments, Ring C is 6-membered heteroaryl.

In certain embodiments, Ring C is 5-membered heteroaryl comprising one nitrogen atom. In certain embodiments, Ring C is 5-membered heteroaryl comprising two nitrogen atoms. In certain embodiments, Ring C is 5-membered heteroaryl comprising three nitrogen atoms. In certain embodiments, Ring C is 5-membered heteroaryl comprising four nitrogen atoms.

In certain embodiments, Ring C is 5-membered heteroaryl comprising at least one nitrogen atom. In certain embodiments, Ring C is 5-membered heteroaryl comprising at least two nitrogen atoms. In certain embodiments, Ring C is 5-membered heteroaryl comprising at least three nitrogen atoms. In certain embodiments, Ring C is 5-membered heteroaryl comprising at least four nitrogen atoms.

In certain embodiments, Ring C is 6-membered heteroaryl comprising one nitrogen atom. In certain embodiments, Ring C is 6-membered heteroaryl comprising two nitrogen atoms. In certain embodiments, Ring C is 6-membered heteroaryl comprising three nitrogen atoms. In certain embodiments, Ring C is 6-membered heteroaryl comprising four nitrogen atoms.

In certain embodiments, Ring C is 6-membered heteroaryl comprising at least one nitrogen atom. In certain embodiments, Ring C is 6-membered heteroaryl comprising at least two nitrogen atoms. In certain embodiments, Ring C is 6-membered heteroaryl comprising at least three nitrogen atoms. In certain embodiments, Ring C is 6-membered heteroaryl comprising at least four nitrogen atoms.

In certain embodiments, all heteroatoms in Ring C are nitrogen.

In certain embodiments, Ring C is oxazole, isoxazole, or triazole. In certain embodiments, Ring C is isoxazole. In certain embodiments, Ring C is triazole. In certain embodiments, Ring C is oxazole.

In certain embodiments, Ring C is 1,2,3-triazole.

In certain embodiments, each R^{C} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{C} is independently hydrogen, C₁₋₆ alkyl, C₃₋₆ carbocyclyl, or —C(═O)R^{a}, wherein the alkyl and carbocyclyl is optionally substituted with one or more R^{u}.

In certain embodiments, c is 0. In certain embodiments, c is 1. In certain embodiments, c is 2. In certain embodiments, c is 3. In certain embodiments, c is 4, as valency permits. In certain embodiments, c is 5, as valency permits.

In certain embodiments, c is 0 or 1.

In certain embodiments, Ring D is C₆ aryl (i.e., phenyl) or 5- to 6-membered heteroaryl (e.g., heteroaryl comprising one 5- or 6-membered ring and 1-4 heteroatoms selected from N, O, and S). In certain embodiments, Ring D is C₆ aryl (i.e., phenyl). In certain embodiments, Ring D is 5-membered heteroaryl. In certain embodiments, Ring D is 6-membered heteroaryl.

In certain embodiments, Ring D is phenyl.

In certain embodiments,

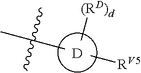

is

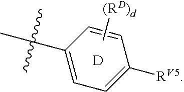

In certain embodiments, each R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, d is 0. In certain embodiments, d is 1. In certain embodiments, d is 2. In certain embodiments, d is 3. In certain embodiments, d is 4.

In certain embodiments, R^{V3} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u}.

In certain embodiments, R^{V3} is hydrogen or C₁₋₆ alkyl. In certain embodiments, R^{V3} is hydrogen. In certain embodiments, R^{V3} is C₁₋₆ alkyl.

In certain embodiments, R^{V4} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), or C₁₋₈ heteroalkyl (e.g., C₁₋₈ heteroalkylene comprising 1-7 heteroatoms selected from N, O, and S), wherein the alkyl or heteroalkyl is optionally substituted with one or more R^{u}.

In certain embodiments, R^{V4} is hydrogen. In certain embodiments, R^{V4} is C₁₋₆ alkyl optionally substituted with one or more R^{u}. In certain embodiments, R^{V4} is C₁₋₆ heteroalkyl optionally substituted with one or more R^{u}. In certain embodiments, R^{V4} is hydrogen, —CH₂C(═O)R^{a}, —CH₂C(═O)OR^{b}, or —CH₂N₃.

In certain embodiments, R^{V5} is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{V5} is halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{V5} is halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{V5} is halogen or 5- to 6-membered heteroaryl (e.g., heteroaryl comprising one 5- or 6-membered ring and 1-3 heteroatoms selected from N, O, and S) optionally substituted with one or more R^{u}.

In certain embodiments, R^{V5} is 5-membered heteroaryl optionally substituted with one or more R^{u}.

In certain embodiments, R^{V5} is thiazolyl optionally substituted with one or more R^{u}.

In certain embodiments, R^{V5} is 6-membered heteroaryl optionally substituted with one or more R^{u}.

In certain embodiments, each R^{a} is independently C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl (CI), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂₋₆ alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{c} and each R^{d} is independently hydrogen, C₁₋₆ alkyl, C₃₋₆ carbocyclyl, or 3- to 6-membered heterocyclyl, wherein the alkyl, carbocyclyl, or heterocyclyls optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, R^{b}, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the compound is selected from the compounds in Table 1 and pharmaceutically acceptable salts thereof.

In certain embodiments, the compound is selected from the compounds in Table 1.

TABLE 1

| Compound | | |
|---|---|---|
| No. | Structure | Chemical Name |
| A1 | 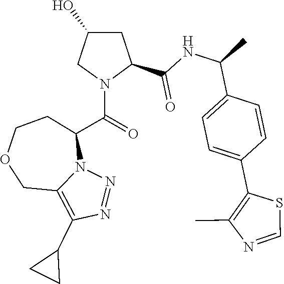 [SMILES: Cc1ncsc1-c1ccc([C@H](C)NC(=O)[C@@H]2C[C@@H](O)CN2C(=O)[C@@H]2CCOCc3c(C4CC4)nnn32)cc1] | (2S,4R)-1-((S)-3-cyclopropyl- 7,8-dihydro-4H,6H-[1,2,3] triazolo[5,1-c][1,4]oxazepine- 8-carbonyl)-4-hydroxy-N-((S)- 1-(4-(4-methyl-thiazol-5-yl)- phenyl)ethyl)pyrrolidine-2- carboxamide |
| A2 | 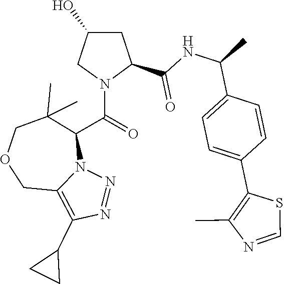 [SMILES: Cc1ncsc1-c1ccc([C@H](C)NC(=O)[C@@H]2C[C@@H](O)CN2C(=O)[C@H]2n3nnc(C4CC4)c3COCC2(C)C)cc1] | (2S,4R)-1-((S)-3-cyclopropyl- 7,7-dimethyl-7,8-dihydro- 4H,6H[1,2,3]triazolo[5,1-c] [1,4]oxazepine-8-carbonyl)-4- hydroxy-N-((S)-1-(4-(4-meth- ylthiazol-5-yl)phenyl)ethyl)- pyrrolidine-2-carboxamide |
| A3 | 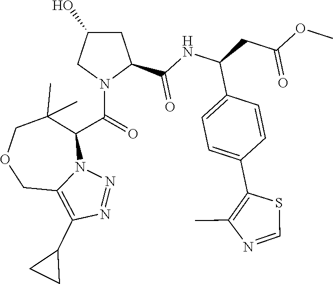 [SMILES: COC(=O)C[C@H](NC(=O)[C@@H]1C[C@@H](O)CN1C(=O)[C@H]1n2nnc(C3CC3)c2COCC1(C)C)c1ccc(-c2scnc2C)cc1] | methyl (S)-3-((2S,4R)-1-((S)- 3-cyclopropyl-7,7-dimethyl- 7,8-dihydro-4H,6H[1,2,3] triazolo[5,1-c][1,4]oxazepine- 8-carbonyl)-4-hydroxypyrrol- idine-2-carboxamido)-3-(4-(4- methylthiazol-5-yl)phenyl)- propanoate |
| A4 | 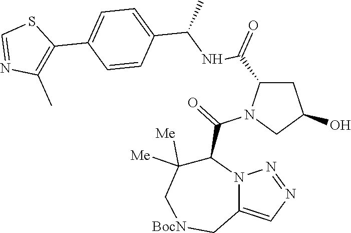 [SMILES: Cc1ncsc1-c1ccc([C@H](C)NC(=O)[C@@H]2C[C@@H](O)CN2C(=O)[C@H]2n3nncc3CN(C(=O)OC(C)(C)C)CC2(C)C)cc1] | tert-butyl (S)-8-((2S,4R)-4- hydroxy-2-(((S)-1-(4-(4-meth- ylthiazol-5-yl)phenyl)ethyl)- carbamoyl)pyrrolidine-1- carbon-yl)-7,7-dimethyl-7,8- dihydro-4H-[1,2,3]triazolo [1,5-a][1,4]diazepine-5(6H)- carboxylate |
| A5 | 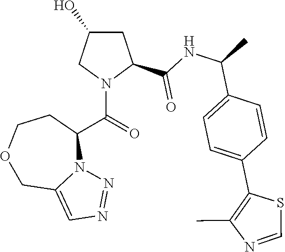 [SMILES: Cc1ncsc1-c1ccc([C@H](C)NC(=O)[C@@H]2C[C@@H](O)CN2C(=O)[C@@H]2CCOCc3cnnn32)cc1] | (2S,4R)-4-hydroxy-N-((S)-1- (4-(4-methylthiazol-5-yl)phen- yl)ethyl)-1-((S)-5,6,7,8-tetra- hydro-4H-[1,2,3]triazolo[1,5- a]azepine-8-carbonyl)pyrrol- idine-2-carboxamide |
| A6 | 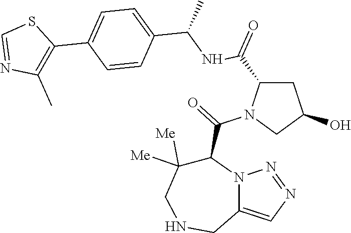 [SMILES: Cc1ncsc1-c1ccc([C@H](C)NC(=O)[C@@H]2C[C@@H](O)CN2C(=O)[C@H]2n3nncc3CNCC2(C)C)cc1] | (2S,4R)-1-((S)-7,7-dimethyl- 5,6,7,8-tetrahydro-4H-[1,2,3] triazolo[1,5-a][1,4]diazepine- 8-carbonyl)-4-hydroxy-N-((S)- 1-(4-(4-methylthiazol-5-yl)- phenyl)ethyl)pyrrolidine-2- carboxamide |
| A7 | 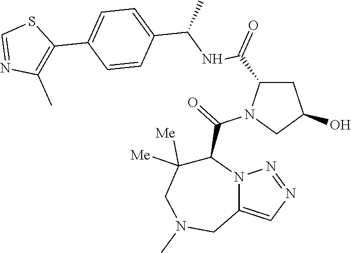 [SMILES: Cc1ncsc1-c1ccc([C@H](C)NC(=O)[C@@H]2C[C@@H](O)CN2C(=O)[C@H]2n3nncc3CN(C)CC2(C)C)cc1] | (2S,4R)-4-hydroxy-N-((S)-1- (4-(4-methylthiazol-5-yl)phen- yl)ethyl)-1-((S)-5,7,7-trimeth- yl-5,6,7,8-tetrahydro-4H-[1,2,3] triazolo[1,5-a][1,4]diazepine- 8-carbonyl)pyrrolidine-2- carboxamide |
| A8 | 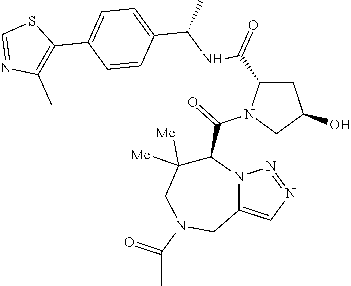 [SMILES: CC(=O)N1Cc2cnnn2[C@H](C(=O)N2C[C@H](O)C[C@H]2C(=O)N[C@@H](C)c2ccc(-c3scnc3C)cc2)C(C)(C)C1] | (2S,4R)-1-((S)-5-acetyl-7,7- dimethyl-5,6,7,8-tetrahydro- 4H-[1,2,3]triazolo[1,5-a][1,4] diazepine-8-carbonyl)-4- hydroxy-N-((S)-1-(4-(4-meth- ylthiazol-5-yl)phenyl)ethyl)- pyrrolidine-2-carboxamide |
| A9 | 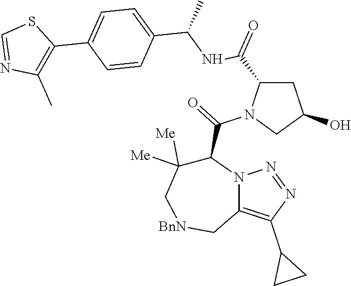 [SMILES: Cc1ncsc1-c1ccc([C@H](C)NC(=O)[C@@H]2C[C@@H](O)CN2C(=O)[C@H]2n3nnc(C4CC4)c3CN(Cc3ccccc3)CC2(C)C)cc1] | (2S,4R)-1-((S)-5-benzyl-3- cyclopropyl-7,7-dimethyl- 5,6,7,8-tetrahydro-4H-[1,2,3] triazolo[1,5-a][1,4]diazepine- 8-carbonyl)-4-hydroxy-N- ((S)-1-(4-(4-methylthiazol-5- yl)phenyl)ethyl)pyrrolidine-2- carboxamide |
| A10 | 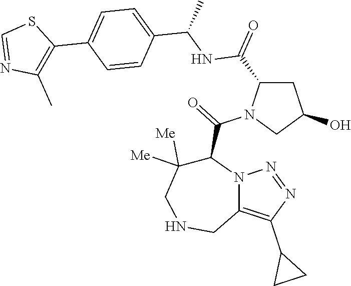 [SMILES: Cc1ncsc1-c1ccc([C@H](C)NC(=O)[C@@H]2C[C@@H](O)CN2C(=O)[C@H]2n3nnc(C4CC4)c3CNCC2(C)C)cc1] | (2S,4R)-1-((S)-3-cyclopropyl- 7,7-dimethyl-5,6,7,8-tetra- hydro-4H-[1,2,3]triazolo[1,5- a][1,4]diazepine-8-carbonyl)- 4-hydroxy-N-((S)-1-(4-(4- methylthiazol-5-yl)phenyl)- ethyl)pyrrolidine-2-carbox- amide |
| A11 | 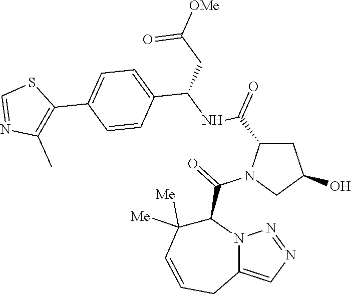 [SMILES: COC(=O)C[C@H](NC(=O)[C@@H]1C[C@@H](O)CN1C(=O)[C@H]1n2nncc2CC=CC1(C)C)c1ccc(-c2scnc2C)cc1] | methyl (S)-3-((2S,4R)-1-((S)- 7,7-dimethyl-7,8-dihydro-4H- [1,2,3]triazolo[1,5-a]azepine- 8-carbonyl)-4-hydroxypyrrol- idine-2-carboxamido)-3-(4-(4- methylthiazol-5-yl)phenyl)- propanoate |
| A12 | 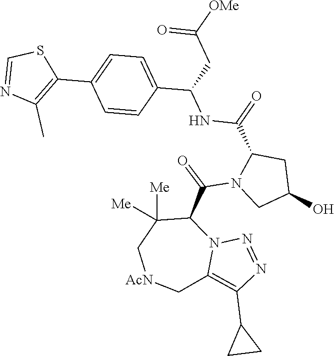 | methyl (S)-3-((2S,4R)-1-((S)- 5-acetyl-3-cyclopropyl-7,7- dimethyl-5,6,7,8-tetrahydro- 4H-[1,2,3]triazolo[1,5-a][1,4] diazepine-8-carbonyl)-4- hydroxypyrrolidine-2-carbox- amido)-3-(4-(4-methylthiazol- 5-yl)phenyl)propanoate |
| A13 | 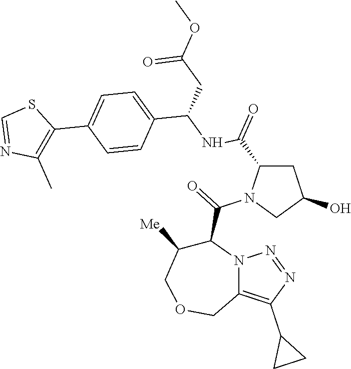 [SMILES: COC(=O)C[C@H](NC(=O)[C@@H]1C[C@@H](O)CN1C(=O)[C@@H]1[C@H](C)COCc2c(C3CC3)nnn21)c1ccc(-c2scnc2C)cc1] | methyl (S)-3-((2S,4R)-1- ((7S,8S)-3-cyclopropyl-7- methyl-7,8-dihydro-4H,6H- [1,2,3]triazolo[5,1-c][1,4] oxazepine-8-carbonyl)-4- hydroxypyrrolidine-2-carbox- amido)-3-(4-(4-methylthiazol- 5-yl)phenyl)propanoate |
| A14 | 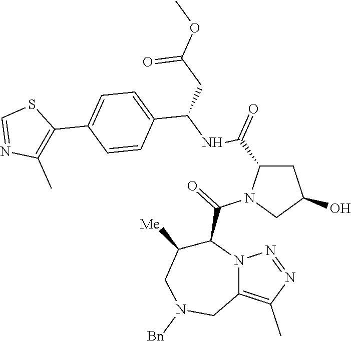 [SMILES: COC(=O)C[C@H](NC(=O)[C@@H]1C[C@@H](O)CN1C(=O)[C@@H]1[C@H](C)CN(Cc2ccccc2)Cc2c(C)nnn21)c1ccc(-c2scnc2C)cc1] | methyl (S)-3-((2S,4R)-1- ((7R,8S)-5-benzyl-3,7-dimeth- yl-5,6,7,8-tetrahydro-4H- [1,2,3]triazolo[1,5-a][1,4] diazepine-8-carbonyl)-4- hydroxypyrrolidine-2-carbox- amido)-3-(4-(4-methylthiazol- 5-yl)phenyl)propanoate |
| A15 | 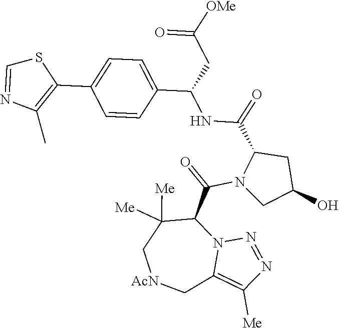 | methyl (S)-3-((2S,4R)-1-((S)- 5-acetyl-3,7,7-trimethyl- 5,6,7,8-tetrahydro-4H-[1,2,3] triazolo[1,5-a][1,4]diazepine- 8-carbonyl)-4-hydroxypyrrol- idine-2-carboxamido)-3-(4-(4- methylthiazol-5-yl)phenyl)- propanoate |
| A16 | 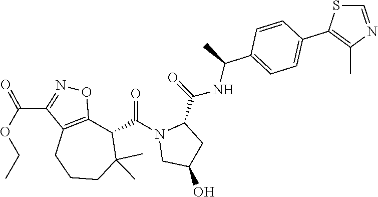 [SMILES: CCOC(=O)c1noc2c1CCCC(C)(C)[C@H]2C(=O)N1C[C@H](O)C[C@H]1C(=O)N[C@@H](C)c1ccc(-c2scnc2C)cc1] | ethyl (R)-8-((2S,4R)-4- hydroxy-2-(((S)-1-(4-(4- methylthiazol-5-yl)phenyl)- ethyl)carbamoyl)pyrrolidine- 1-carbonyl)-7,7-dimethyl- 5,6,7,8-tetrahydro-4H-cyclo- hepta[d]isoxazole-3-carbox- ylate |
| A17 | 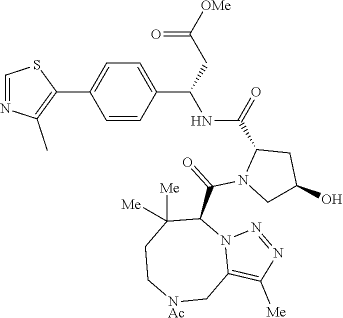 | methyl (S)-3-((2S,4R)-1-((S)- 5-acetyl-3,8,8-trimethyl- 4,5,6,7,8,9-hexahydro-[1,2,3] triazolo[1,5-a][1,4]diazocine- 9-carbonyl)-4-hydroxypyrrol- idine-2-carboxamido)-3-(4-(4- methylthiazol-5-yl)phenyl)- propanoate |
| A18 | 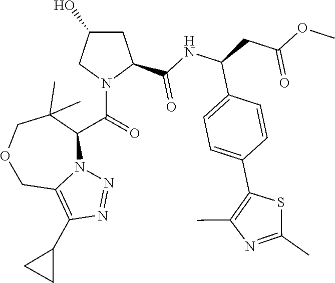 [SMILES: COC(=O)C[C@H](NC(=O)[C@@H]1C[C@@H](O)CN1C(=O)[C@H]1n2nnc(C3CC3)c2COCC1(C)C)c1ccc(-c2sc(C)nc2C)cc1] | methyl (S)-3-((2S,4R)-1-((S)- 3-cyclopropyl-7,7-dimethyl- 7,8-dihydro-4H,6H-[1,2,3] triazolo[5,1-c][1,4]oxazepine- 8-carbonyl)-4-hydroxypyrrol- idine-2-carboxamido)-3-(4- (2,4-dimethylthiazol-5-yl)- phenyl)propanoate |
| A19 | 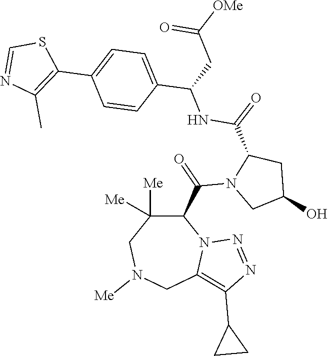 [SMILES: COC(=O)C[C@H](NC(=O)[C@@H]1C[C@@H](O)CN1C(=O)[C@H]1n2nnc(C3CC3)c2CN(C)CC1(C)C)c1ccc(-c2scnc2C)cc1] | methyl (S)-3-((2S,4R)-1-((S)- 3-cyclopropyl-5,7,7-trimethyl- 5,6,7,8-tetrahydro-4H-[1,2,3] triazolo[1,5-a][1,4]diazepine- 8-carbonyl)-4-hydroxypyrrol- idine-2-carboxamido)-3-(4-(4- methylthiazol-5-yl)phenyl)- propanoate |
| A20 | 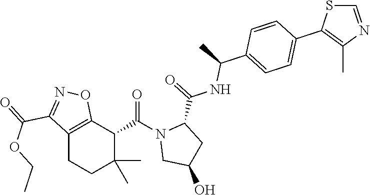 [SMILES: CCOC(=O)c1noc2c1CCC(C)(C)[C@H]2C(=O)N1C[C@H](O)C[C@H]1C(=O)N[C@@H](C)c1ccc(-c2scnc2C)cc1] | ethyl (R)-7-((2S,4R)-4- hydroxy-2-(((S)-1-(4-(4-meth- ylthiazol-5-yl)phenyl)ethyl)- carbamoyl)pyrrolidine-1- carbonyl)-6,6-dimethyl- 4,5,6,7-tetrahydrobenzo[d] isoxazole-3-carboxylate |
| A21 | 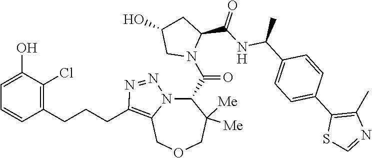 [SMILES: Cc1ncsc1-c1ccc([C@H](C)NC(=O)[C@@H]2C[C@@H](O)CN2C(=O)[C@H]2n3nnc(CCCc4cccc(O)c4Cl)c3COCC2(C)C)cc1] | (2S,4R)-1-((S)-3-(3-(2-chloro- 3-hydroxyphenyl)propyl)-7,7- dimethyl-7,8-dihydro-4H,6H- [1,2,3]triazolo[5,1-c][1,4] oxazepine-8-carbonyl)-4- hydroxy-N-((S)-1-(4-(4-meth- ylthiazol-5-yl)phenyl)ethyl)- pyrrolidine-2-carboxamide |
| A22 | 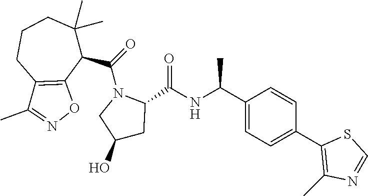 [SMILES: Cc1ncsc1-c1ccc([C@H](C)NC(=O)[C@@H]2C[C@@H](O)CN2C(=O)[C@H]2c3onc(C)c3CCCC2(C)C)cc1] | (2S,4R)-4-hydroxy-N-((S)-1- (4-(4-methylthiazol-5-yl)phen- yl)ethyl)-1-((R)-3,7,7-trimeth- yl-5,6,7,8-tetrahydro-4H- cyclohepta[d]isoxazole-8- carbonyl)pyrrolidine-2- carboxamide |
| A23 | 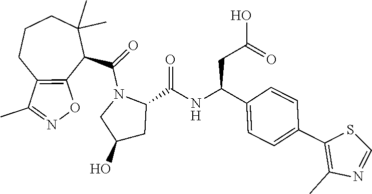 [SMILES: Cc1ncsc1-c1ccc([C@H](CC(=O)O)NC(=O)[C@@H]2C[C@@H](O)CN2C(=O)[C@H]2c3onc(C)c3CCCC2(C)C)cc1] | methyl (S)-3-((2S,4R)-4- hydroxy-1-((R)-3,7,7-trimeth- yl-5,6,7,8-tetrahydro-4H- cyclohepta[d]isoxazole-8- carbonyl)pyrrolidine-2-carbox- amido)-3-(4-(4-methylthiazol- 5-yl)phenyl)propanoate |
| A24 | 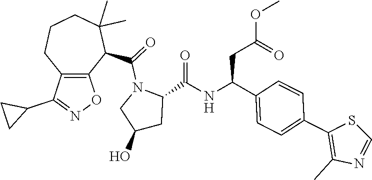 [SMILES: COC(=O)C[C@H](NC(=O)[C@@H]1C[C@@H](O)CN1C(=O)[C@H]1c2onc(C3CC3)c2CCCC1(C)C)c1ccc(-c2scnc2C)cc1] | methyl (S)-3-((2S,4R)-1-((R)- 3-cyclopropyl-7,7-dimethyl- 5,6,7,8-tetrahydro-4H-cyclo- hepta[d]isoxazole-8-carbon- yl)-4-hydroxypyrrolidine-2- carboxamido)-3-(4-(4-methyl- thiazol-5-yl)phenyl)propanoate |
| A25 | 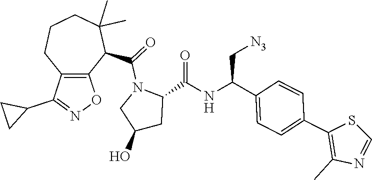 [SMILES: Cc1ncsc1-c1ccc([C@H](CN=[N+]=[N-])NC(=O)[C@@H]2C[C@@H](O)CN2C(=O)[C@H]2c3onc(C4CC4)c3CCCC2(C)C)cc1] | (2S,4R)-N-((R)-2-azido-1-(4- (4-methylthiazol-5-yl)phen- yl)ethyl)-1-((R)-3-cycloprop- yl-7,7-dimethyl-5,6,7,8-tetra- hydro-4H-cyclohepta[d]isox- azole-8-carbonyl)-4-hydroxy- pyrrolidine-2-carboxamide |
| A26 | 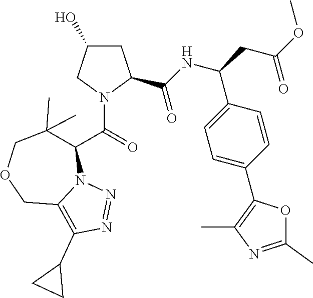 [SMILES: COC(=O)C[C@H](NC(=O)[C@@H]1C[C@@H](O)CN1C(=O)[C@H]1n2nnc(C3CC3)c2COCC1(C)C)c1ccc(-c2oc(C)nc2C)cc1] | methyl (S)-3-((2S,4R)-1-((S)- 3-cyclopropyl-7,7-dimethyl- 7,8-dihydro-4H,6H-[1,2,3] triazolo[5,1-c][1,4]oxazepine- 8-carbonyl)-4-hydroxypyrrol- idine-2-carboxamido)-3-(4- (2,4-dimethyloxazol-5-yl)- phenyl)propanoate |

### Bifunctional Degraders

In certain aspects, the present disclosure provides conjugates comprising a compound disclosed herein being connected to a ligand for a protein (e.g., via a linker).

In certain aspects, the present disclosure provides conjugates of Formula I:

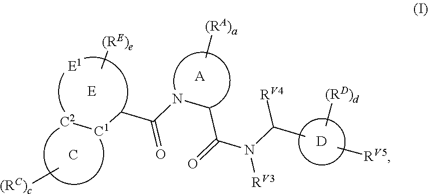

and pharmaceutically acceptable salts, solvates, or stereoisomers thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

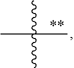

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

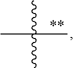

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

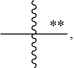

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

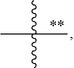

hydrogen, C₆₋₁₀ alkyl, C₁₋₈ heteroalkyl, wherein the alkyl, C₆₋₁₀ alkylene, or heteroalkyl is optionally substituted with one or more R^{u};
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is

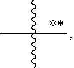

wherein

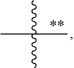

denotes attachment to -L-T,
L is a linker; and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₁₂ carbocyclyl, 3- to 12-membered heterocyclyl, C₆₋₁₀ aryl, or 5- to 10-membered heteroaryl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.
wherein each of the variables in Formula I is described herein.

In certain embodiments, the conjugate is a conjugate of Formula I-0 or I-0′

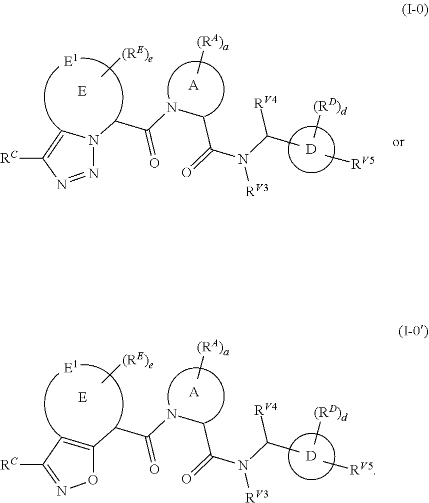

In certain embodiments, the conjugate is a conjugate of Formula I-1 or I-2

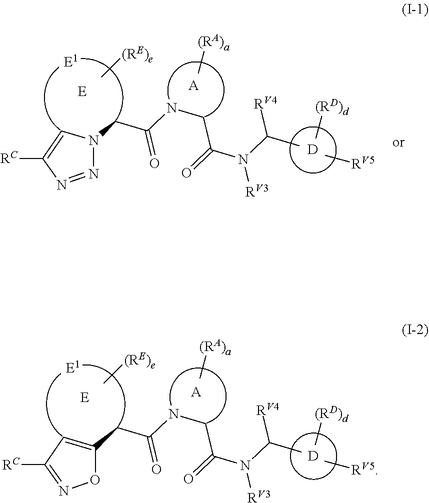

In certain embodiments, the conjugate is a conjugate of Formula I-1-i or I-2-i

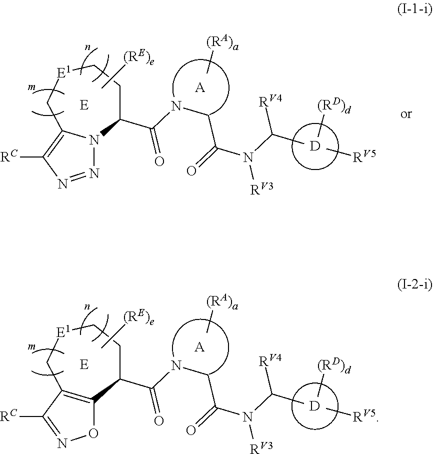

In certain embodiments, the conjugate is a conjugate of Formula I-1-ii or I-2-ii

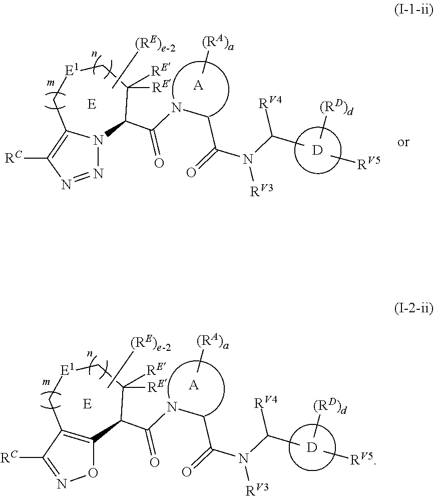

In certain embodiments, the ligand portion of the degrader, prior to conjugation, is selected from Table 1.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrades the target protein.

In certain embodiments, L is a linker comprising C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted by one or more R^{u}.

In certain embodiments, L is of Formula I-2

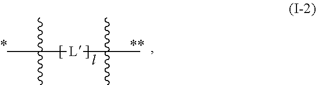

wherein:
* denotes attachment to T and ** denotes attachment to C;
each L′ is independently C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L2})—, —C(═O)O—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₃₋₁₂ carbocyclylene, 3- to 12-membered heterocyclylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each occurrence of R^{L′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{u} is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, 1 is 0. In certain embodiments, t is 1. In certain embodiments, 1 is 2. In certain embodiments, 1 is 3. In certain embodiments, 1 is 4. In certain embodiments, 1 is 5. In certain embodiments, 1 is 6.

T, a ligand of a protein, is a chemical entity that competitively or non-competitively binds a protein.

In certain embodiments, the protein is B7.1 and B7, TINFR1m, TNFR2, NADPH oxidase, BclIBax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300(E1A) binding protein (CBP/p300), SWI/SNF Related, Matrix Associated, Actin Dependent Regulator Of Chromatin, Subfamily A, Member 2/4 (SMARCA2/4), Ikaros Zinc Finger (IKZF)1, IKZF2, or IKZF3, Kirsten rat sarcoma viral oncogene homolog G12D (KRAS G12D), Src homology region 2-containing protein tyrosine phosphatase 2 (SHP2), bromodomain-containing protein 4 (BRD4), or bromodomain-containing protein 9 (BRD9).

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is a ligand for SMARCA2/4 protein. In certain embodiments, T is a ligand for STAT3 protein. In certain embodiments, T is a ligand for CBP/p300 protein. In certain embodiments, T is a ligand for Ikaros Zinc Finger (IKZF)1, IKZF2, or IKZF3. In certain embodiments, T is ligand for an androgen receptor. In certain embodiments, T is a ligand for BRD9 protein.

In certain embodiments, T is an estrogen receptor inhibitor. In certain embodiments, T is a SMARCA2/4 protein inhibitor. In certain embodiments, T is a STAT3 protein inhibitor. In certain embodiments, T is a CBP/p300 protein inhibitor. In certain embodiments, T is a Ikaros Zinc Finger (IKZF)1, IKZF2, or IKZF3 degrader. In certain embodiments, T is an androgen receptor inhibitor. In certain embodiments, T is a BRD9 protein inhibitor.

In certain aspects, the present disclosure provides conjugates of Formula II:

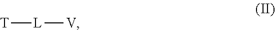

and pharmaceutically acceptable salts, solvates, or stereoisomers thereof, wherein:
V is of Formula II-1

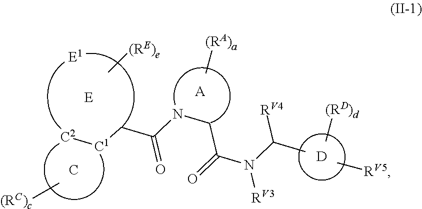

wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

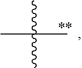

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NRSC(═O)₂R^{a}, —NR^{c}S(═O)R^{a}, —NR^{c}(═O)₂OR^{b},—NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

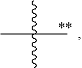

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E}; E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

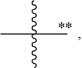

oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

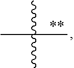

hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is

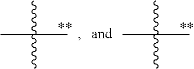

and denotes attachment to -L-T,
L is of Formula II-2

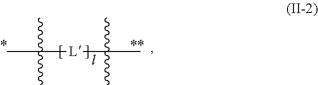

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6,

### T is of Formula II-3

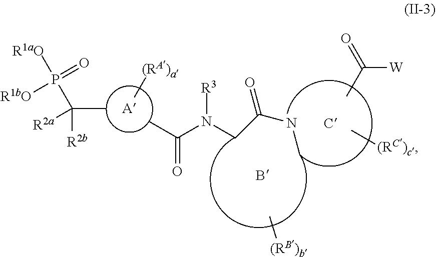

wherein:
R^{1a} and R^{1b} are independently hydrogen or C₁₋₆ alkyl optionally substituted with one or more R^{u};
R^{2a} and R^{2b} are independently hydrogen or halogen;
R^{2a} and R^{2b} together an oxo; or
R^{2a} and R^{2b}, together with the carbon atom to which they are attached, form a C₃₋₆ carbocycle or 3- to 6-membered heterocycle, wherein the carbocycle or heterocycle is optionally substituted with one or more R^{u};
Ring A′ is C₆₋₁₀ aryl or 5- to 10-membered heteroaryl;
each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 6, as valency permits;
R³ is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring B′ is 3- to 12-membered heterocycle;
each R^{B′} is independently

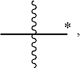

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b},—NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
b′ is an integer selected from 0 to 6, as valency permits;
Ring C′ is 3- to 8-membered heterocycle;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 6, as valency permits;
W is

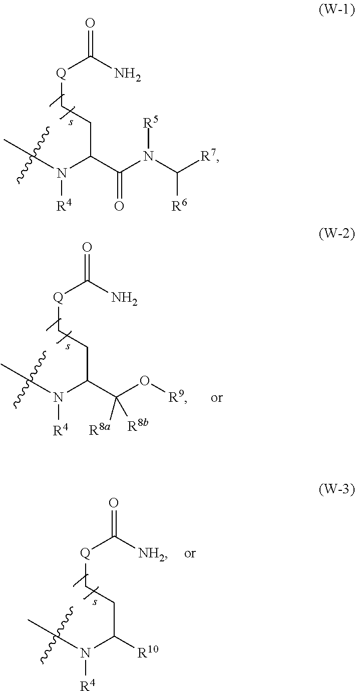

W is

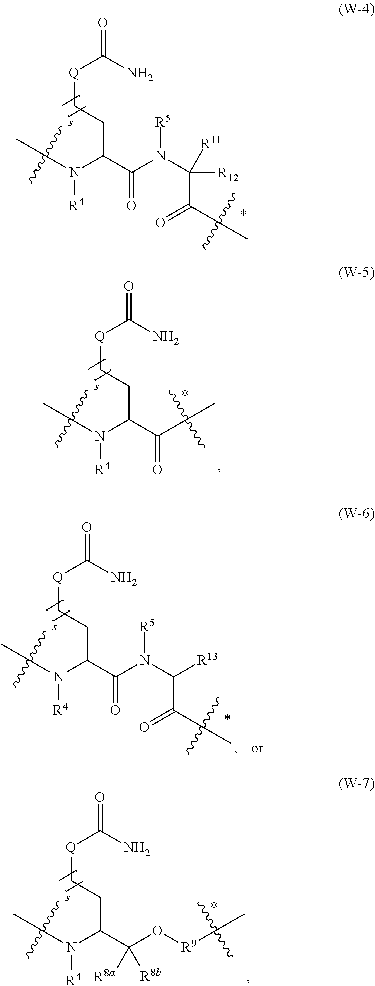

wherein:
* denotes attachment to L;
Q is absent; or
Q is —C(R^{Q})₂—, —O—, and —N(R^{Q′})—;
each R^{Q} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{Q}, together with the carbon atom to which they are attached, form C₃₋₆ carbocyclyl or 3- to 6-membered heterocyclyl, wherein the carbocyclyl or heterocyclyl is optionally substituted with one or more R^{u};
R^{Q′} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u};
s is an integer selected from 0 to 4;
R⁴ and R⁵ are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R⁶ and R⁷ are independently C₆₋₁₀ aryl or 5- to 10-membered heteroaryl, wherein the aryl or heteroaryl is optionally substituted with one or more R^{6a} or R^{7a}, respectively;
each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R⁷ is hydrogen or C₁₋₆ alkyl optionally substituted with one or more R^{7b};
each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
R⁶ and R⁷, together with the carbon atom to which they are attached, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
R^{8a} and R^{8b} are independently hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, carbocyclyl, or heterocyclyl is optionally substituted with one or more R^{u};
R⁹ is C₆₋₁₀ aryl or 5- to 10-membered heteroaryl, wherein the aryl or heteroaryl is optionally substituted with one or more R^{9a};
each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹⁰ is hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkenyl, alkynyl, alkoxy, alkylamino, carbocyclyl, heterocyclyl, aryl, or heteroaryl is optionally substituted with one or more R^{u}; R^{u} is hydrogen or C₁₋₆ alkyl optionally substituted with one or more R^{u};
R¹² is hydrogen, C₁₋₆ alkyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a};
each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹¹ and R¹², together with the carbon atom to which they are attached, form C₃₋₆ carbocyclyl or 3- to 6-membered heterocyclyl, wherein carbocyclyl or heterocyclyl is optionally substituted with one or more R^{u}; or
R¹¹ and R⁵, together with the intervening atoms, form 4- to 8-membered heterocyclyl optionally substituted with one or more R^{u}; and
R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, V is of Formula II-1

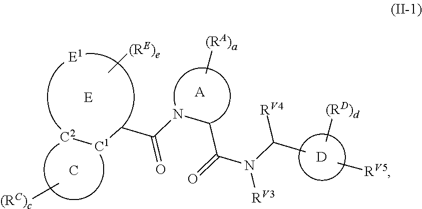

wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

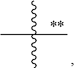

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

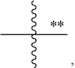

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

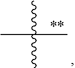

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

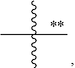

hydrogen, C₆₋₁₀ alkyl, C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is

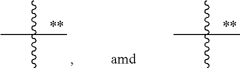

and denotes attachment to -L-T.

In certain embodiments, V is of Formula II-1-i or II-1-ii

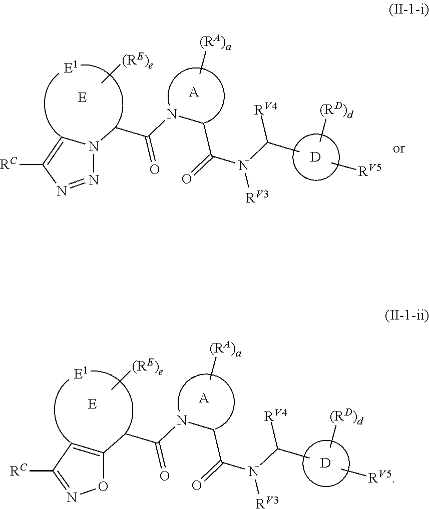

In certain embodiments, V is of Formula II-1-iii or II-1-iv

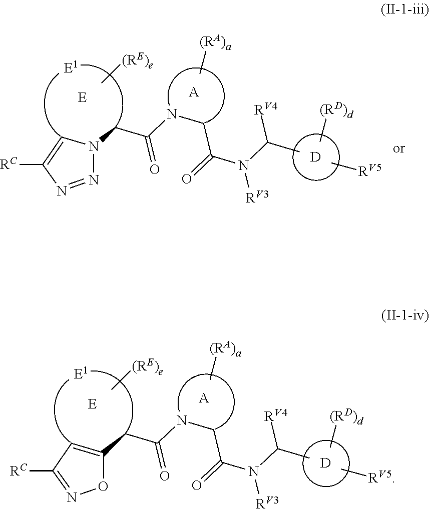

In certain embodiments, V is of Formula II-1-v or II-1-vi

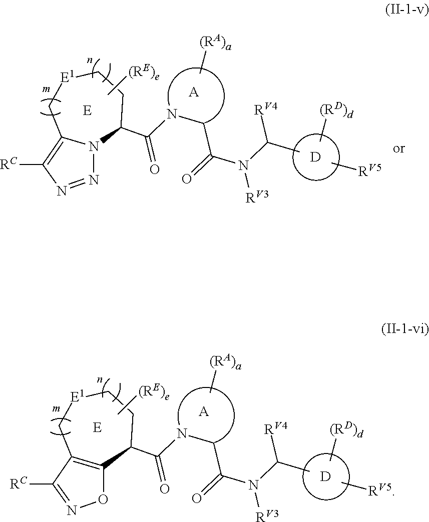

In certain embodiments, V is of Formula II-1-vii or II-1-viii

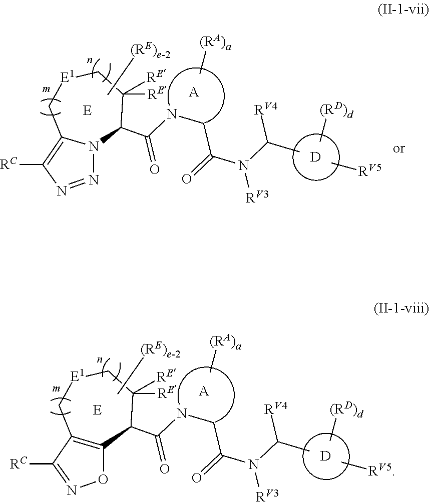

In certain embodiments, V is of Formula II-1-vii or II-1-viii

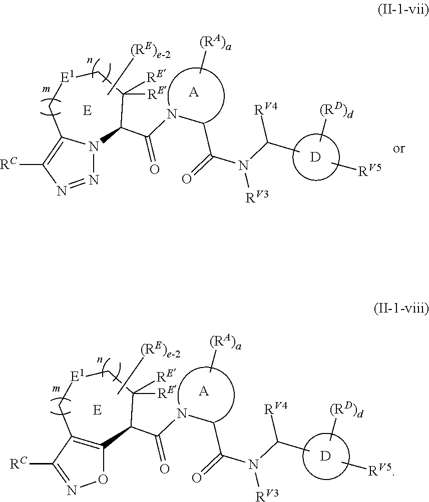

wherein each of R^{E′} is independently C₁₋₆ alkyl.

In certain embodiments, m is 1 or 2; and n is 0 or 1.

In certain embodiments, m is 1. In certain embodiments, m is 2.

In certain embodiments, n is 0. In certain embodiments, n is 1.

In certain embodiments, m is 1 and n is 1.

In certain embodiments, m is 2 and n is 1.

In certain embodiments, m is 1 and n is 0.

In certain embodiments, Ring E is C₅₋₁₂ carbocyclyl (e.g., cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 5- to 12-membered heterocyclyl (e.g., heterocyclyl comprising one or two 5- to 8-membered rings and 1-5 heteroatoms selected from N, O, and S), wherein the carbocyclyl or heterocyclyl is optionally substituted with one or more R^{u}.

In certain embodiments, each R^{E} is independently oxo, hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{E} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least two R^{E} are independently C₁₋₆ alkyl.

In certain embodiments, two geminal R^{E}, together with the carbon atom to which they are bonded, form C₃₋₆ carbocycle (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocycle (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, e is 0. In certain embodiments, e is 1. In certain embodiments, e is 2. In certain embodiments, e is 3. In certain embodiments, e is 4. In certain embodiments, e is 5. In certain embodiments, e is 6.

In certain embodiments, e is an integer selected from 2 to 6.

In certain embodiments, each R^{E′} is independently hydrogen, C₁₋₆ alkyl, or C₁₋₆ alkoxy (e.g., methyl (CI), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), wherein at least one R^{E′} is not hydrogen.

In certain embodiments, each R^{E′} is independently C₁₋₆ alkyl. In certain embodiments, each R^{E′} is independently methyl, ethyl, or propyl. In certain embodiments, each R^{E′} is methyl.

In certain embodiments, two R^{E′}, together with the carbon atom to which they are bonded, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, R^{E} is R^{E′}.

In certain embodiments, E¹ is C(R^{E1′})₂, NR^{E1″}, O, or S. In certain embodiments, E¹ is C(R^{E1′})₂, NR^{E1″}, O, or S. In certain embodiments, E¹ is C(R^{E1′})₂. In certain embodiments, E¹ is NR^{E1″}. In certain embodiments, E¹ is 0. In certain embodiments, E¹ is S.

In certain embodiments, each R^{E1′} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkoxy, alkylamino, carbocyclyl, or heterocyclyl is optionally substituted with one or more R^{u}.

In certain embodiments, R^{E1′} is hydrogen or C₁₋₆ alkyl.

In certain embodiments, R^{E1″} is an amino-protecting group (e.g., carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)), hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u}.

In certain embodiments, R^{E1″} is amino-protecting group, hydrogen, C₁₋₆ alkyl, or —C(═O)R^{a}.

In certain embodiments, V is of Formula II-1-ix, II-1-x, II-1-xi, II-1-xii, or II-1-xiii:

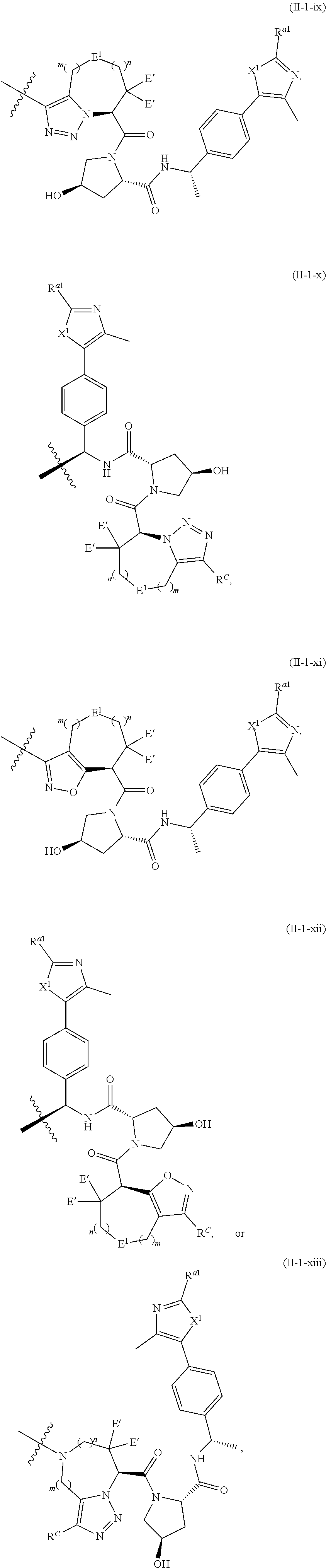

wherein:
R^{a1} is C₆₋₁₀ alkyl;
X¹ is O or S;
E¹ is CH₂, NR^{E1″} or O;
R^{E1″} is hydrogen, C₁₋₆ alkyl, or —C(═O)R^{a};
E′ is hydrogen or C₁₋₆ alkyl;
R^{a} is C₁₋₆ alkyl;
R^{C} is C₆₋₁₀ alkyl or C₃₋₆ carbocyclyl;
m is 1 or 2; and
n is 0 or 1.

In certain embodiments, V is of Formula II-1-xiv, II-1-xv, or II-1-xvi:

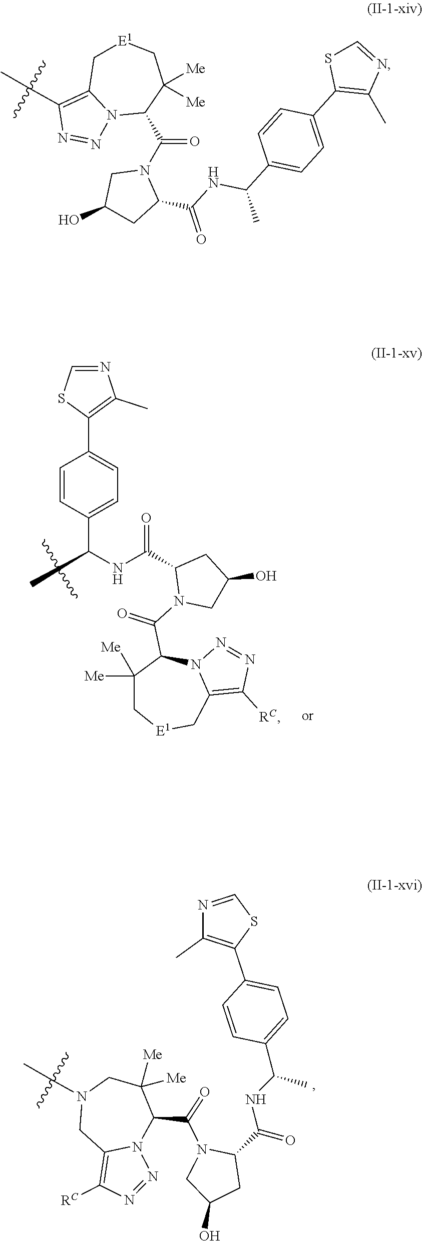

wherein:
E¹ is CH₂, NR^{E1″} or O;
R^{E1″} is hydrogen, C₆₋₁₀ alkyl, or —C(═O)R^{a};
R^{a} is methyl;
R^{C} is C₆₋₁₀ alkyl or C₃₋₆ carbocyclyl.

In certain embodiments, V is of Formula II-1-xiv or II-1-xv.

In certain embodiments, V is of Formula II-1-xiv or II-1-xv, and E¹ is 0.

In certain embodiments, V is of Formula II-1-xvii or II-1-xviii:

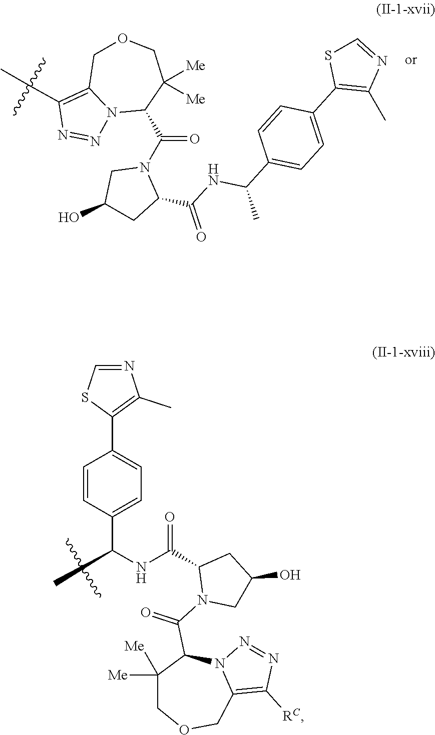

wherein R^{c} is methyl or cyclopropyl.

In certain embodiments, Ring A is 5- to 7-membered heterocycle. In certain embodiments, Ring A is 5-membered heterocycle. In certain embodiments, Ring A is 6-membered heterocycle. In certain embodiments, Ring A is 7-membered heterocycle.

In certain embodiments, each R^{A} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH-2, C₁₋₆ alkyl, C₁₋₆ alkoxy, C₁₋₆ alkylamino, C₂₋₆ alkenyl, C₂₋₆ alkynyl, C₃₋₆ carbocyclyl, or 3 to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, at least one R^{A} is —OH.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 8, as valency permits. In certain embodiments, a is 9, as valency permits. In certain embodiments, a is 10, as valency permits.

In certain embodiments, a is 1 or 2, and at least one of R^{A} is —OH.

In certain embodiments,

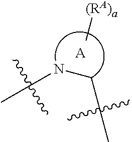

is

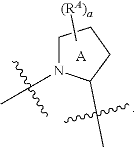

In certain embodiments,

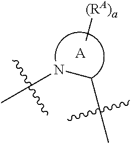

is

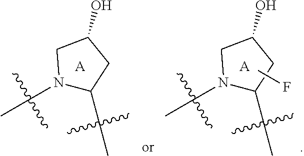

In certain embodiments, Ring C is C₆ aryl (i.e., phenyl) or 5- to 6-membered heteroaryl (e.g., heteroaryl comprising one 5- or 6-membered ring and 1-4 heteroatoms selected from N, O, and S).

In certain embodiments, Ring C is C₆ aryl (i.e., phenyl). In certain embodiments, Ring C is 5-membered heteroaryl. In certain embodiments, Ring C is 6-membered heteroaryl.

In certain embodiments, Ring C is 5-membered heteroaryl comprising one nitrogen atom. In certain embodiments, Ring C is 5-membered heteroaryl comprising two nitrogen atoms. In certain embodiments, Ring C is 5-membered heteroaryl comprising three nitrogen atoms. In certain embodiments, Ring C is 5-membered heteroaryl comprising four nitrogen atoms.

In certain embodiments, Ring C is 5-membered heteroaryl comprising at least one nitrogen atom. In certain embodiments, Ring C is 5-membered heteroaryl comprising at least two nitrogen atoms. In certain embodiments, Ring C is 5-membered heteroaryl comprising at least three nitrogen atoms. In certain embodiments, Ring C is 5-membered heteroaryl comprising at least four nitrogen atoms.

In certain embodiments, Ring C is 6-membered heteroaryl comprising one nitrogen atom. In certain embodiments, Ring C is 6-membered heteroaryl comprising two nitrogen atoms. In certain embodiments, Ring C is 6-membered heteroaryl comprising three nitrogen atoms. In certain embodiments, Ring C is 6-membered heteroaryl comprising four nitrogen atoms.

In certain embodiments, Ring C is 6-membered heteroaryl comprising at least one nitrogen atom. In certain embodiments, Ring C is 6-membered heteroaryl comprising at least two nitrogen atoms. In certain embodiments, Ring C is 6-membered heteroaryl comprising at least three nitrogen atoms. In certain embodiments, Ring C is 6-membered heteroaryl comprising at least four nitrogen atoms.

In certain embodiments, all heteroatoms in Ring C must be nitrogen.

In certain embodiments, Ring C is oxazole, isoxazole, or triazole. In certain embodiments, Ring C is isoxazolel. In certain embodiments, Ring C is triazole. In certain embodiments, Ring C is 1,2,3-triazole.

In certain embodiments, each R^{C} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3 to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{C} is independently hydrogen, C₁₋₆ alkyl, or C₃₋₆ carbocyclyl.

In certain embodiments, c is 0. In certain embodiments, c is 1. In certain embodiments, c is 2. In certain embodiments, c is 3. In certain embodiments, c is 4, as valency permits. In certain embodiments, c is 5, as valency permits.

In certain embodiments, c is 0 or 1.

In certain embodiments, Ring D is C₆ aryl (i.e., phenyl) or 5- to 6-membered heteroaryl (e.g., heteroaryl comprising one 5- or 6-membered ring and 1-4 heteroatoms selected from N, O, and S). In certain embodiments, Ring D is C₆ aryl (i.e., phenyl). In certain embodiments, Ring D is 5-membered heteroaryl. In certain embodiments, Ring D is 6-membered heteroaryl.

In certain embodiments, Ring D is phenyl.

In certain embodiments,

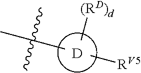

is

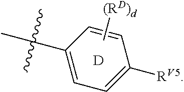

In certain embodiments, each R^{D} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{D} is independently halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, wherein the alkyl, alkoxy, alkylamino, carbocyclyl, or heterocyclyl is optionally substituted with one or more R^{u}.

In certain embodiments, d is 0. In certain embodiments, d is 1. In certain embodiments, d is 2. In certain embodiments, d is 3. In certain embodiments, d is 4.

In certain embodiments, R^{V2} is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}. In certain embodiments, R^{V2} is hydrogen. In certain embodiments, R^{V2} is C₁₋₆ alkyl optionally substituted with one or more R^{u}.

In certain embodiments, R^{V2} is 2-propyl or t-butyl.

In certain embodiments, R^{V2} is C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)) or 3- to 12-membered heterocyclyl (e.g., heterocyclyl comprising one or two 3- to 8-membered rings and 1-5 heteroatoms selected from N, O, and S), wherein the carbocyclyl or heterocyclyl is attached to L and optionally substituted with one or more R^{u}.

In certain embodiments, one R^{C} and R^{V2}, together with the intervening atoms, form 12 carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)) or 3- to 12-membered heterocyclyl (e.g., heterocyclyl comprising one or two 3- to 8-membered rings and 1-5 heteroatoms selected from N, O, and S), wherein the carbocyclyl or heterocyclyl is optionally substituted with one or more R^{u}.

In certain embodiments, R^{V3} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3 to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u}.

In certain embodiments, R^{V3} is hydrogen or C₁₋₆ alkyl. In certain embodiments, R^{V3} is hydrogen. In certain embodiments, R^{V3} is C₁₋₆ alkyl.

In certain embodiments, R^{V4} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₈ heteroalkyl (e.g., C₁₋₈ heteroalkylene comprising 1-7 heteroatoms selected from N, O, and S), wherein the alkyl or heteroalkyl is optionally substituted with one or more R^{u}. In certain embodiments, R¹⁴ is hydrogen. In certain embodiments, R^{V4} is C₁₋₆ alkyl optionally substituted with one or more R^{u}. In certain embodiments, R^{V4} is methyl. In certain embodiments, R^{V4} is C₁₋₆ heteroalkyl optionally substituted with one or more R^{u}. In certain embodiments, R^{V4} is MeO(CH₂O)CH₂—.

In certain embodiments, R^{V5} is hydrogen, halogen (e.g., —F, —Cl, —Br, or —I), —CN, —NO₂, —OH, —NH₂, C₁₋₆ alkyl (e.g., methyl (CI), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, 5- to 6-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, —C(═O)OR^{b}, or —C(═O)NR^{c}R^{d}, wherein the alkyl, alkoxy, alkylamino, alkenyl, alkynyl, carbocyclyl, or heterocyclyl is optionally substituted with one or more R^{u}.

In certain embodiments, R^{V5} is hydrogen, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, —C(═O)OR^{b}, or —C(═O)NR^{c}R^{d}, wherein the alkyl, alkoxy, alkylamino, carbocyclyl, or heterocyclyl is optionally substituted with one or more R^{u}.

In certain embodiments, R^{V5} is halogen or 5- to 6-membered heteroaryl (e.g., heteroaryl comprising one 5- or 6-membered ring and 1-3 heteroatoms selected from N, O, and S) optionally substituted with one or more R^{u}. In certain embodiments, R^{V5} is 5-membered heteroaryl optionally substituted with one or more R^{u}. In certain embodiments, R^{V5} is thiazolyl optionally substituted with one or more R^{u}. In certain embodiments, R^{V5} is 6-membered heteroaryl optionally substituted with one or more R^{u}.

In certain embodiments, L is of Formula II-2

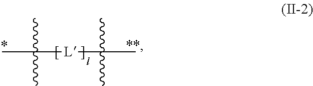

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₁₋₆ heteroalkylene (e.g., C₁₋₆ heteroalkylene comprising 1-7 heteroatoms selected from N, O, and S), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L2})—, —C(═O)O—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₁₋₆ heteroalkylene (e.g., C₁₋₆ heteroalkylene comprising 1-7 heteroatoms selected from N, O, and S), C₃₋₁₂ carbocyclylene, 3- to 12-membered heterocyclylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₁₋₆ heteroalkylene, 5 to 10-membered heteroarylene, C₃₋₁₂ carbocyclylene, 3- to 12-membered heterocyclylene, —C(═O)—, —C(═O)N(R^{L′})—, —C(═O)O—, —N(R^{L′})—, —O—, or —S(═O)₂—, wherein the alkylene, heteroalkylene, heteroarylene, carbocyclylene, or heterocyclylene is optionally substituted with one or more R^{u}.

In certain embodiments, L′ is C₁₋₆ alkylene, C₁₋₆ heteroalkylene, C₃₋₁₂ carbocyclylene, 3- to 12-membered heterocyclylene, —C(═O)—, —N(R^{L′})—, or —O—, wherein the alkylene, carbocyclylene, or heterocyclylene is optionally substituted with one or more R^{u}.

In certain embodiments, each L′ is independently C₁₋₆ alkylene, —C(═O)—, or —N(R^{L′})—.

In certain embodiments, each occurrence of R^{L′} is independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L′} is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, 1 is 0. In certain embodiments, 1 is 1. In certain embodiments, 1 is 2. In certain embodiments, 1 is 3. In certain embodiments, 1 is 4. In certain embodiments, 1 is 5. In certain embodiments, 1 is 6.

In certain embodiments, L is C₁₋₆ alkylene, —(C₁₋₆ alkylene)-N(R^{L′})C(═O)—, or —(C₁₋₆ alkylene)-N(R^{L′})C(═O)—(C₁₋₆ alkylene)-, wherein the alkylene is optionally substituted with one or more R^{u}.

In certain embodiments, L is C₁₋₆ alkylene, —(C₁₋₆ alkylene)-(5- to 10-membered heteroarylene)-(C₁₋₆ alkylene)-, —(C₁₋₆ alkylene)-N(R^{L′})C(═O)—, —C(═O)—(C₁₋₆ alkylene)-N(R^{L′})C(═O)₁(C₁₋₆ alkylene)-, or —(C₁₋₆ alkylene)-N(R^{L′})C(═O)(C₁₋₆ alkylene)-, wherein the alkylene is optionally substituted with one or more R^{u}.

In certain embodiments, T is of Formula II-3

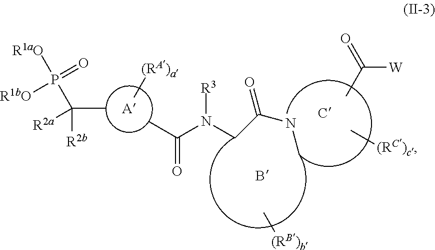

wherein:
R^{1a} and R^{1b} are independently hydrogen or C₁₋₆ alkyl optionally substituted with one or more R^{u}.
R^{2a} and R^{2b} are independently hydrogen or halogen;
R^{2a} and R^{2b} together an oxo; or
R^{2a} and R^{2b}, together with the carbon atom to which they are attached, form C₃₋₆ carbocycle or 3- to 6-membered heterocycle, wherein the carbocycle or heterocycle is optionally substituted with one or more R^{u};
Ring A′ is C₆₋₁₀ aryl or 5- to 10-membered heteroaryl;
each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 6, as valency permits;
R³ is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring B′ is 3- to 12-membered heterocycle;
each R^{B′} is independently

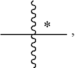

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
b′ is an integer selected from 0 to 6, as valency permits;
Ring C′ is 3- to 8-membered heterocycle;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 6, as valency permits;
W is

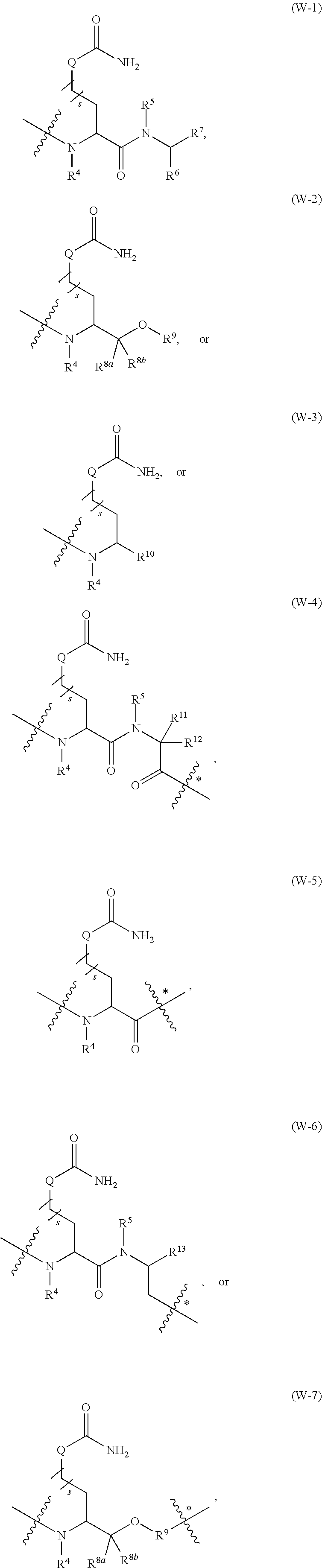

W is

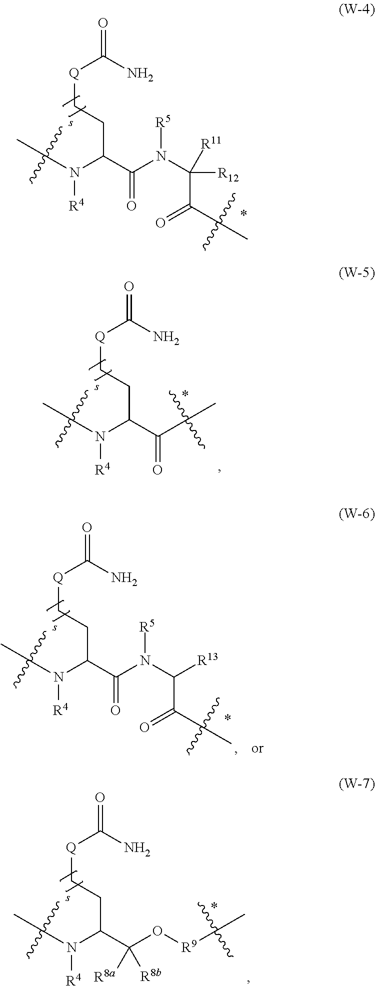

wherein:
* denotes attachment to L;
Q is absent; or
Q is —C(R^{Q})₂—, —O—, and —N(R^{Q′})—;
each R^{Q} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{Q}, together with the carbon atom to which they are attached, form C₃₋₆ carbocyclyl or 3 to 6-membered heterocyclyl, wherein the carbocyclyl or heterocyclyl is optionally substituted with one or more R^{u};
R^{Q′} is hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u};
s is an integer selected from 0 to 4;
R⁴ and R⁵ are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R⁶ and R⁷ are independently C₆₋₁₀ aryl or 5- to 10-membered heteroaryl, wherein the aryl or heteroaryl is optionally substituted with one or more R^{6a} or R^{7a}, respectively;
each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R⁷ is hydrogen or C₁₋₆ alkyl optionally substituted with one or more R^{7b};
each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
R⁶ and R⁷, together with the carbon atom to which they are attached, form C₃₋₁₂ carbocyclyl, 3 to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
R^{8a} and R^{8b} are independently hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, carbocyclyl, or heterocyclyl is optionally substituted with one or more R^{u};
R⁹ is C₆₋₁₀ aryl or 5- to 10-membered heteroaryl, wherein the aryl or heteroaryl is optionally substituted with one or more R^{9a};
each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹⁰ is hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkenyl, alkynyl, alkoxy, alkylamino, carbocyclyl, heterocyclyl, aryl, or heteroaryl is optionally substituted with one or more R^{u};
R¹¹ is hydrogen or C₁₋₆ alkyl optionally substituted with one or more R^{u};
R¹² is hydrogen, C₁₋₆ alkyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a};
each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹¹ and R¹², together with the carbon atom to which they are attached, form C₃₋₆ carbocyclyl or 3- to 6-membered heterocyclyl, wherein carbocyclyl or heterocyclyl is optionally substituted with one or more R^{u}; or
R¹¹ and R⁵, together with the intervening atoms, form 4- to 8-membered heterocyclyl optionally substituted with one or more R^{u}; and
R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, T is of Formula II-3-i-a or II-3-i-b

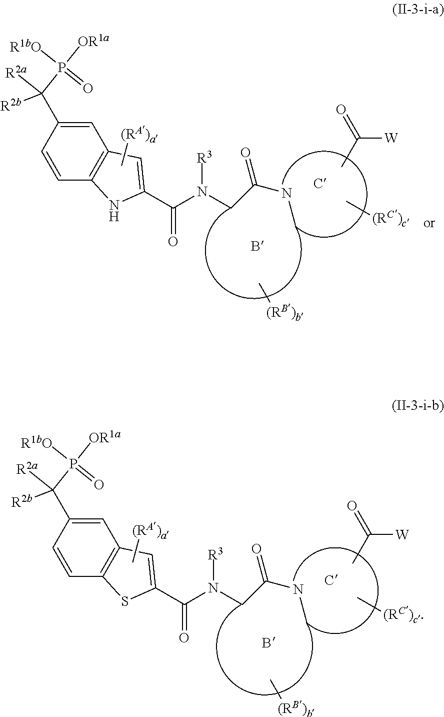

In certain embodiments, T is of Formula II-3-ii-a or II-3-ii-b

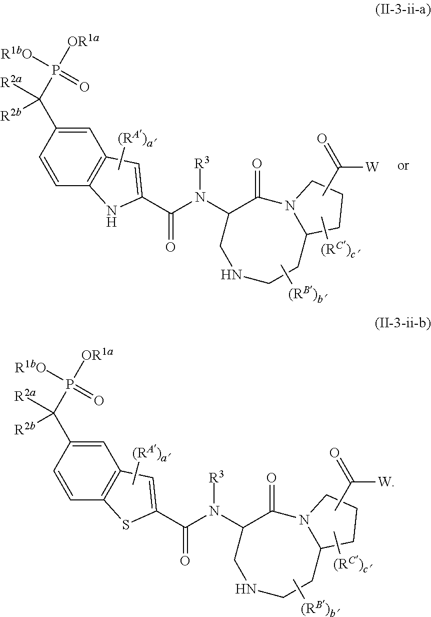

In certain embodiments, T is of Formula II-3-ii-c or II-3-ii-d:

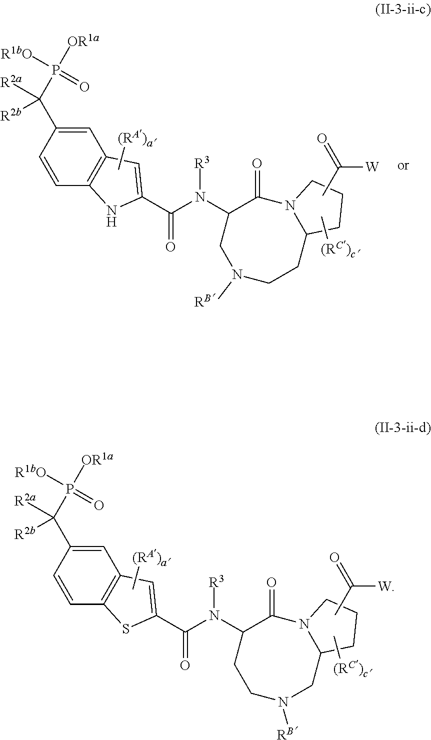

In certain embodiments, T is of Formula II-3-ii-e or II-3-ii-f:

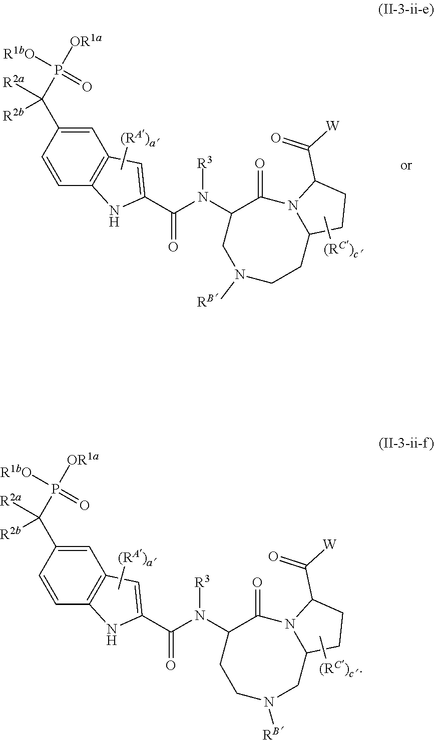

In certain embodiments, T is of Formula II-3-ii-g or II-3-ii-h:

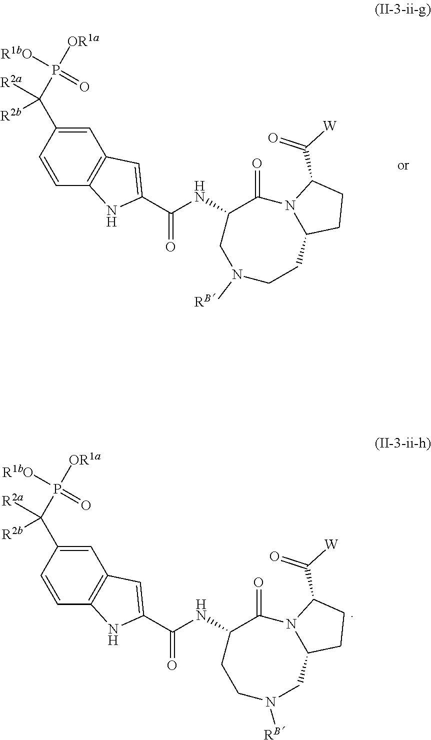

In certain embodiments, T is of Formula II-3-i-i or II-3-ii-j:

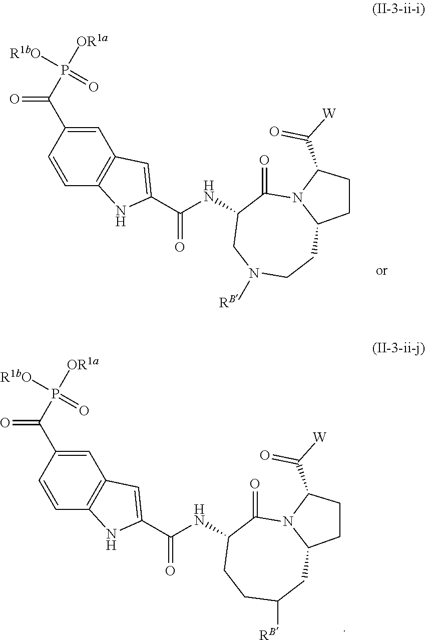

In certain embodiments, R^{1a} and R^{1b} are independently hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}.

In certain embodiments, R^{2a} and R^{2b} are independently hydrogen or halogen (e.g., —F, —Cl, —Br, or —I). In certain embodiments, R^{2a} and R^{2b} are independently halogen. In certain embodiments, at least one of R^{2a} and R^{2b} is halogen. In certain embodiments, R^{2a} and R^{2b} are both —F. In certain embodiments, R^{2a} and R^{2b} are both —C₁.

In certain embodiments, R^{2a} and R^{2b} together an oxo.

In certain embodiments, R^{2a} and R^{2b}, together with the carbon atom to which they are attached, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S).

In certain embodiments, Ring A′ is C₆₋₁₀ aryl (e.g., phenyl or naphthyl) or 5- to 10-membered heteroaryl (e.g., heteroaryl comprising one or two 5- or 6-membered rings and 1-5 heteroatoms selected from N, O, and S).

In certain embodiments, Ring A′ is

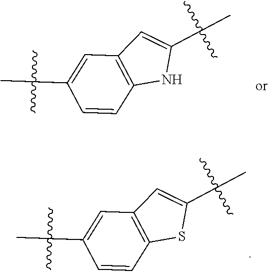 [SMILES: *c1ccc2[nH]c(*)cc2c1 ; *c1ccc2sc(*)cc2c1]

In certain embodiments, each R^{A′} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, a′ is 0. In certain embodiments, a′ is 1. In certain embodiments, a′ is 2. In certain embodiments, a′ is 3, as valency permits. In certain embodiments, a′ is 4, as valency permits. In certain embodiments, a′ is 5, as valency permits. In certain embodiments, a′ is 6, as valency permits.

In certain embodiments, R³ is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R³ is hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R³ is hydrogen, C₁₋₆ alkyl, C₂₋₆ alkenyl, C₂₋₆ alkynyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R³ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R³ is hydrogen.

In certain embodiments, Ring B′ is 3- to 12-membered heterocycle (e.g., heterocyclyl comprising one or two 3- to 8-membered rings and 1-5 heteroatoms selected from N, O, and S).

In certain embodiments,

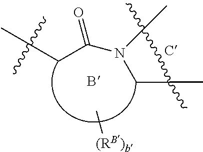

is

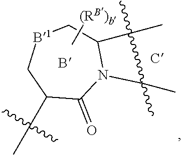

wherein B′¹ is —NHCH₂—, —CH₂NH—, or —CH₂CH₂—.

In certain embodiments,

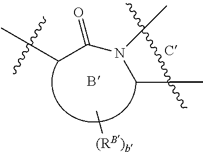

is

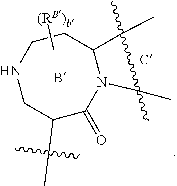

In certain embodiments, each R^{B′} is independently

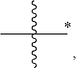

oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B′} is independently

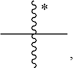

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B′} is independently

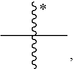

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{B′} is independently

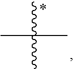

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{B′} is independently

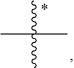

halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, b′ is 0. In certain embodiments, b′ is 1. In certain embodiments, b′ is 2. In certain embodiments, b′ is 3, as valency permits. In certain embodiments, b′ is 4, as valency permits. In certain embodiments, b′ is 5, as valency permits. In certain embodiments, b′ is 6, as valency permits.

In certain embodiments, each R^{B′} is independently

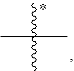

—C(═O)R^{a}, or —C(═O)OR^{b}.

In certain embodiments, at least one R^{B′} is

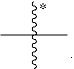

In certain embodiments, at least one R^{B′} is —C(═O)R^{a}. In certain embodiments, at least one R^{B′} is —C(═O)OR^{b}.

In certain embodiments, R^{B′} is —C(═O)CH₃.

In certain embodiments, R^{B′} is —C(═O)CH₃ and W is W-7.

In certain embodiments, R^{B′} is

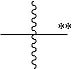

### and W is W-8.

In certain embodiments, Ring C′ is 3- to 8-membered heterocycle (e.g., heterocyclyl comprising one or two 3- to 8-membered rings and 1-3 heteroatoms selected from N, O, and S).

In certain embodiments, each R^{C′} is independently

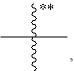

halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C′} is independently

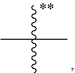

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C′} is independently

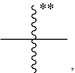

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{C′} is independently

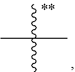

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{C′} is independently

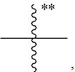

halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, c′ is 0. In certain embodiments, c′ is 1. In certain embodiments, c′ is 2. In certain embodiments, c′ is 3, as valency permits. In certain embodiments, c′ is 4, as valency permits. In certain embodiments, c′ is 5, as valency permits. In certain embodiments, c′ is 6, as valency permits.

In certain embodiments,
W is

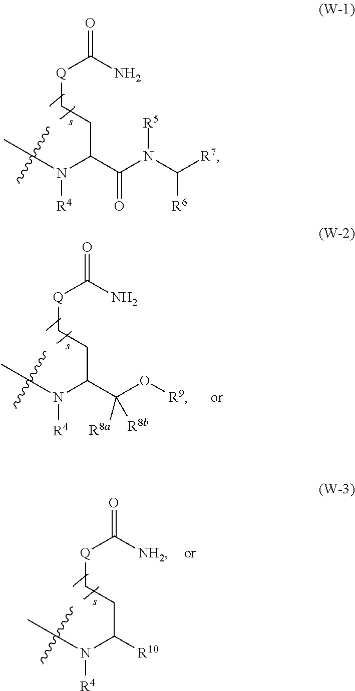

W is

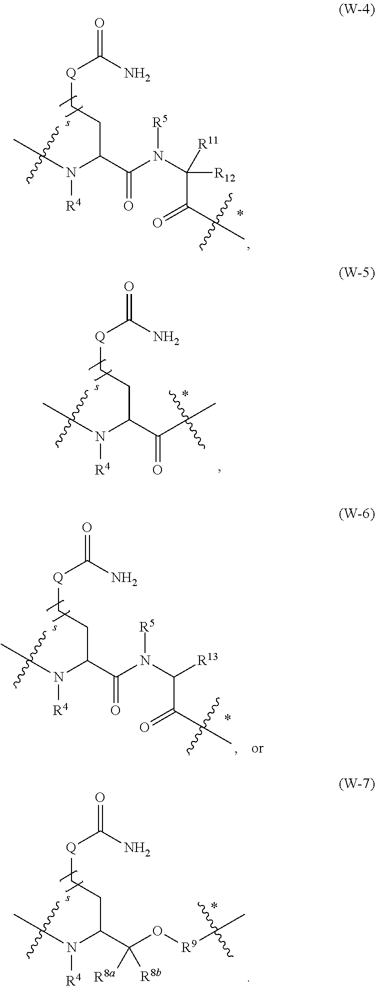

In certain embodiments, W is W-7 or W-8:

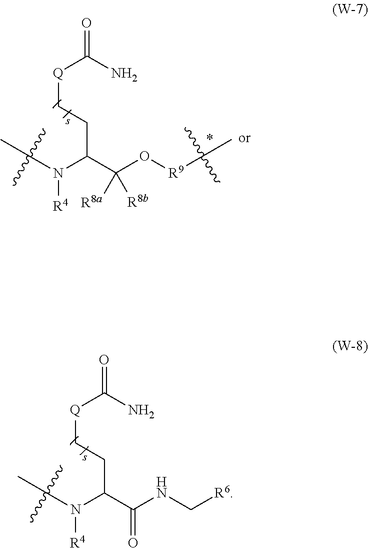

In certain embodiments, Q is absent. In certain embodiments, Q is —C(R^{Q})₂—, —O—, and —N(R^{Q′})—.

In certain embodiments, each R^{Q} is independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), or 3- to 6-membered heterocyclyl (e.g., heterocyclyl comprising one 3- to 6-membered ring and 1-3 heteroatoms selected from N, O, and S), wherein the alkyl, alkoxy, alkylamino, carbocyclyl, or heterocyclyl is optionally substituted with one or more R^{u}.

In certain embodiments, two R^{Q}, together with the carbon atom to which they are connected, form C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{u}.

In certain embodiments, R^{Q′} is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₆ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), or cyclohexadienyl (C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u}.

In certain embodiments, s is 0. In certain embodiments, s is 1. In certain embodiments, s is 2. In certain embodiments, s is 3. In certain embodiments, s is 4.

In certain embodiments, R⁴ and R⁵ are independently hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R⁴ and R⁵ are independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R⁴ and R⁵ are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R⁴ and R⁵ are both hydrogen.

In certain embodiments, R⁶ and R⁷ are independently C₆₋₁₀ aryl (e.g., phenyl or naphthyl) or 5- to 10-membered heteroaryl (e.g., heteroaryl comprising one or two 5- or 6-membered rings and 1-5 heteroatoms selected from N, O, and S), wherein the aryl or heteroaryl is optionally substituted with one or more R^{6a} or R^{7b}, respectively.

In certain embodiments, R⁶ is unsubstituted. In certain embodiments, R⁶ is substituted with one R^{6a}. In certain embodiments, R⁶ is substituted with two R^{6a}. In certain embodiments, R⁶ is substituted with three R^{6a}. In certain embodiments, R⁶ is substituted with four R^{6a}. In certain embodiments, R⁶ is substituted with five R^{6a}.

In certain embodiments, R⁷ is unsubstituted. In certain embodiments, R⁷ is substituted with one R^{7a}. In certain embodiments, R⁷ is substituted with two R^{7a}. In certain embodiments, R⁷ is substituted with three R^{7a}. In certain embodiments, R⁷ is substituted with four R^{7a}. In certain embodiments, R⁷ is substituted with five R^{7a}.

In certain embodiments, each R^{6a} and each R^{7a} are independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{6a} and each R^{7a} are independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{6a} and each R^{7a} are independently halogen, C₁₋₆ alkyl, C₁₋₆ alkylamino, or —S(═O)₂R^{a}, wherein the alkyl or alkylamino is optionally substituted with one or more R^{u}.

In certain embodiments, at least one R^{6a} is halogen. In certain embodiments, at least one R^{6a} is C₁₋₆ alkyl, wherein the alkyl is optionally substituted with one or more R^{u}. In certain embodiments, at least one R^{6a} is C₁₋₆ alkylamino, wherein the alkylamino is optionally substituted with one or more R^{u}. In certain embodiments, at least one R^{6a} is —S(═O)₂R^{a}.

In certain embodiments, at least one R^{7a} is halogen. In certain embodiments, at least one R^{7a} is C₁₋₆ alkyl, wherein the alkyl is optionally substituted with one or more R^{u}. In certain embodiments, at least one R^{7a} is C₁₋₆ alkylamino, wherein the alkylamino is optionally substituted with one or more R^{u}. In certain embodiments, at least one R^{7a} is —S(═O)₂R^{a}.

In certain embodiments, R⁷ is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{7b}.

In certain embodiments, each R^{7b} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁), or spiro[4.5]decanyl (C₁)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{7b} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R⁶ and R⁷, together with the carbon atom to which they are attached, form C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the carbocyclyl, heterocyclyl, aryl, or heteroaryl is optionally substituted with one or more R^{u}.

In certain embodiments, W is W-8;
R⁶ is C₆₋₁₀ aryl, wherein the aryl is optionally substituted with one or more R^{6a};
each R^{6a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d};
R^{a} is C₁₋₆ alkyl, C₃₋₁₂ carbocyclyl, 3- to 12-membered heterocyclyl, C₆₋₁₀ aryl, or 5- to 10-membered heteroaryl;
R^{b} is hydrogen, C₁₋₆ alkyl, C₃₋₁₂ carbocyclyl, 3- to 12-membered heterocyclyl, C₆₋₁₀ aryl, or 5- to 10-membered heteroaryl;
R^{c} and R^{d} are independently hydrogen, C₁₋₆ alkyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl.

In certain embodiments, R^{8a} and R^{8b} are independently hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, carbocyclyl, heterocyclyl, aryl, or heteroaryl is optionally substituted with one or more R^{u}.

In certain embodiments, R^{8a} and R^{8b} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{8a} and R^{8b} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{8a} and R^{8b} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{8a} and R^{8b} are both hydrogen.

In certain embodiments, R⁹ is C₆₋₁₀ aryl (e.g., phenyl or naphthyl) or 5- to 10-membered heteroaryl (e.g., heteroaryl comprising one or two 5- or 6-membered rings and 1-5 heteroatoms selected from N, O, and S), wherein the aryl or heteroaryl is optionally substituted with one or more R^{7b}.

In certain embodiments, R⁹ is unsubstituted. In certain embodiments, R⁹ is substituted with one R^{9a}. In certain embodiments, R⁹ is substituted with two R^{9a}. In certain embodiments, R⁹ is substituted with three R^{9a}. In certain embodiments, R⁹ is substituted with four R^{9a}. In certain embodiments, R⁹ is substituted with five R^{9a}.

In certain embodiments, R⁹ is:

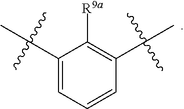

In certain embodiments, each R^{9a} is independently halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{9a} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{9a} is independently halogen or —S(═O)₂R^{a}. In certain embodiments, at least one R^{9a} is independently halogen. In certain embodiments, at least one R^{9a} is independently —S(═O)₂R^{a}.

In certain embodiments, R¹⁰ is hydrogen, halogen (e.g., —F, —Cl, —Br, or —I), —CN, —NO₂, —OH, —NH₂, C₁₋₆ alkyl (e.g., methyl (CI), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, carbocyclyl, or heterocyclyl is optionally substituted with one or more R^{u}.

In certain embodiments, R¹⁰ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R¹⁰ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R¹⁰ is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{u} is hydrogen or C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)) optionally substituted with one or more R^{u}. In certain embodiments, R^{u} is hydrogen.

In certain embodiments, R¹² is hydrogen, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a}.

In certain embodiments, R¹² is —(C₁₋₆ alkylene)-(C₆₋₁₀ aryl) or —(C₁₋₆ alkylene)-(5- to 10-membered heteroaryl), wherein the alkylene is optionally substituted with one or more R^{u}, and aryl or heteroaryl is optionally substituted with one or more R^{12a}.

In certain embodiments, R¹² is unsubstituted. In certain embodiments, R¹² is substituted with one R^{12a}. In certain embodiments, R¹² is substituted with two R^{12a}. In certain embodiments, R¹² is substituted with three R^{12a}. In certain embodiments, R¹² is substituted with four R^{12a}. In certain embodiments, R¹² is substituted with five R^{12a}.

In certain embodiments, each R^{12a} is independently oxo, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₅), cyclooctenyl (C₅), bicyclo[2.2.1]heptanyl (C₇), bicyclo[2.2.2]octanyl (C₅), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, carbocyclyl, heterocyclyl, aryl, or heteroaryl is optionally substituted with one or more R^{u}.

In certain embodiments, each R^{12a} is independently oxo, halogen, —CN, —NO₂, —OH, —NH₂, Ct-6 alkyl, Ct-6 alkoxy, Ct-6 alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{12a} is independently oxo, halogen, —CN, —NO₂, —OH, —NH₂, C₁₋₆ alkyl, Ct-6 alkoxy, Ct-6 alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{12a} is independently oxo, halogen, —CN, —NO₂, —OH, —NH₂, Ct-6 alkyl, Ct-6 alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each R^{12a} is independently halogen, C₆₋₁₀ alkyl, C₁₋₆ alkylamino, or —S(═O)₂R^{a}, wherein the alkyl or alkylamino is optionally substituted with one or more R^{u}. In certain embodiments, at least one R^{12a} is halogen. In certain embodiments, at least one R^{12a} is C₁₋₆ alkyl, wherein the alkyl is optionally substituted with one or more R^{u}. In certain embodiments, at least one R^{12a} is C₁₋₆ alkylamino, wherein the alkylamino is optionally substituted with one or more R^{u}. In certain embodiments, at least one R^{12a} is —S(═O)₂R^{a}.

In certain embodiments, R¹¹ and R¹², together with the carbon atom to which they are attached, form C₃₋₆ carbocyclyl (e.g., cyclopropyl (C₃), or cyclopropenyl (C₃)) or 3- to 6-membered heterocyclyl (e.g., heterocyclyl comprising one 3- to 6-membered ring and 1-3 heteroatoms selected from N, O, and S), wherein carbocyclyl or heterocyclyl is optionally substituted with one or more R^{u}.

In certain embodiments, R¹¹ and R⁵ taken together form 4- to 8-membered heterocyclyl (e.g., heterocyclyl comprising one or two 4- to 8-membered rings and 1-5 heteroatoms selected from N, O, and S) optionally substituted with one or more R^{u}.

In certain embodiments, R¹³ is hydrogen, halogen (e.g., —F, —Cl, —Br, or —I), —CN, —NO₂, —OH, —NH₂, C₁₋₆ alkyl (e.g., methyl (C₁), ethyl (C₂), n-propyl (C₃), i-propyl (C₃), n-butyl (C₄), i-butyl (C₄), s-butyl (C₄), t-butyl (C₄), pentyl (C₅), or hexyl (C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl (C₂), 1-propenyl (C₃), 2-propenyl (C₃), 1-butenyl (C₄), 2-butenyl (C₄), butadienyl (C₄), pentenyl (C₅), pentadienyl (C₅), or hexenyl (C₆)), C₂₋₆ alkynyl (e.g., ethynyl (C₂), 1-propynyl (C₃), 2-propynyl (C₃), 1-butynyl (C₄), 2-butynyl (C₄), pentynyl (C₅), or hexynyl (C₆)), C₃₋₁₂ carbocyclyl (e.g., cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), cyclononyl (C₉), cyclononenyl (C₉), cyclodecyl (C₁₀), cyclodecenyl (C₁₀), octahydro-1H-indenyl (C₉), decahydronaphthalenyl (C₁₀), or spiro[4.5]decanyl (C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R¹³ is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments,
L is C₁₋₆ alkylene;
T is of Formula II-3-ii-a

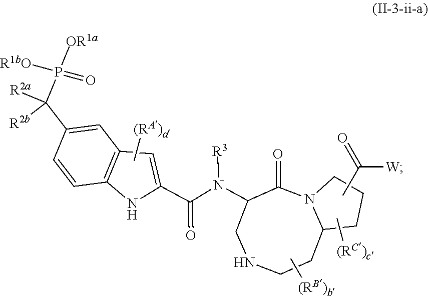

wherein W is

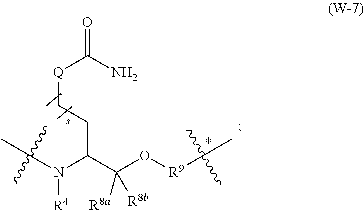

and
V is of Formula II-1-vii

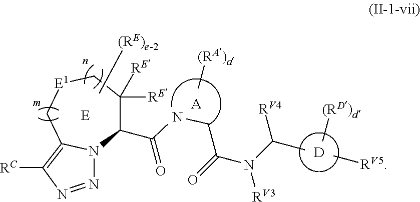

In certain embodiments, the conjugate is of Formula II-X

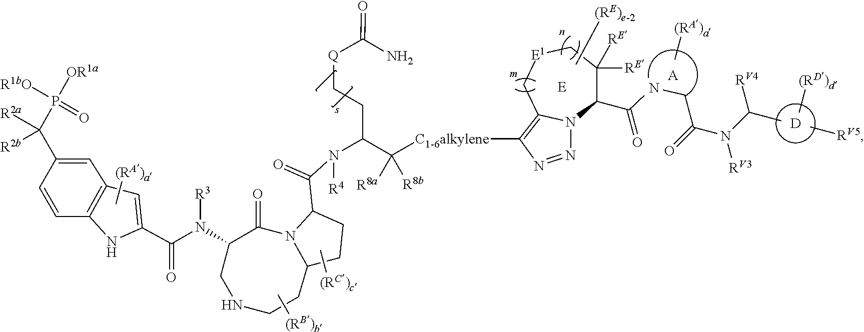

or a pharmaceutically acceptable salt, solvate, or stereoisomer thereof.

In certain embodiments, the conjugate is of Formula II-XI:

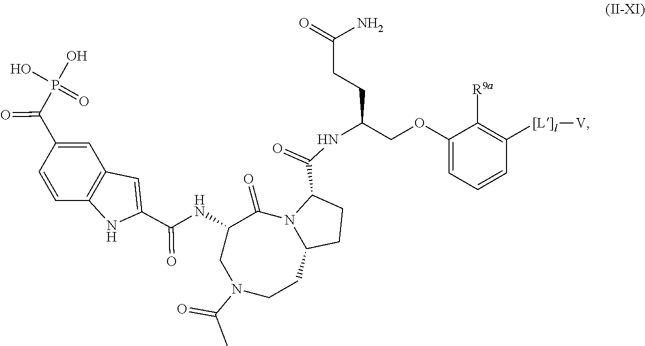

or a pharmaceutically acceptable salt thereof, wherein:
R^{9a} is halogen, e.g., chloro;
each L′ is independently C₁₋₆ alkylene, 5- to 10-membered heteroarylene, —C(═O)—, —C(═O)N(R^{L′})—, —C(═O)O—, or —N(R^{L′})—;
R^{L′} is hydrogen;
L is an integer from 1 to 4; and
V is of Formula II-1-ix, II-1-x, II-1-xi, II-1-xii, or II-1-xiii.

In certain embodiments, the conjugate is of Formula II-XI, or a pharmaceutically acceptable salt thereof, wherein V is of Formula II-1-xiv, II-1-xv, or II-1-xvi.

In certain embodiments, the conjugate is of Formula II-XI, or a pharmaceutically acceptable salt thereof, wherein V is of Formula II-1-xvii or II-1-xviii.

In certain embodiments, the conjugate is of Formula II-XI, or a pharmaceutically acceptable salt thereof, wherein V is:

[chemical structure]

In certain embodiments, the conjugate is of Formula II-XII:

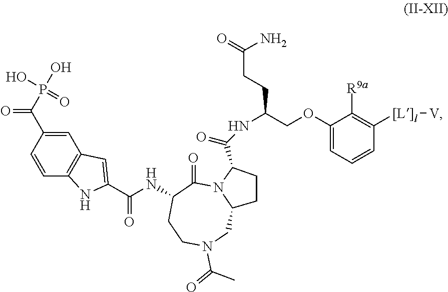

or a pharmaceutically acceptable salt thereof, wherein:
R^{9a} is halogen, e.g., chloro;
each L′ is independently C₁₋₆ alkylene, 5- to 10-membered heteroarylene, —C(═O)—, —C(═O)N(R^{L′})—, —C(═O)O—, or —N(R^{L′})—;
R^{L′} is hydrogen;
L is an integer from 1 to 4; and
V is of Formula II-1-ix, II-1-x, II-1-xi, II-1-xii, or II-1-xiii.

In certain embodiments, the conjugate is of Formula II-XII, or a pharmaceutically acceptable salt thereof, wherein V is of Formula II-1-xiv, II-1-xv, or II-1-xvi.

In certain embodiments, the conjugate is of Formula II-XII, or a pharmaceutically acceptable salt thereof, wherein V is of Formula II-1-xvii or II-1-xviii.

In certain embodiments, the conjugate is of Formula II-XII, or a pharmaceutically acceptable salt thereof, wherein V is:

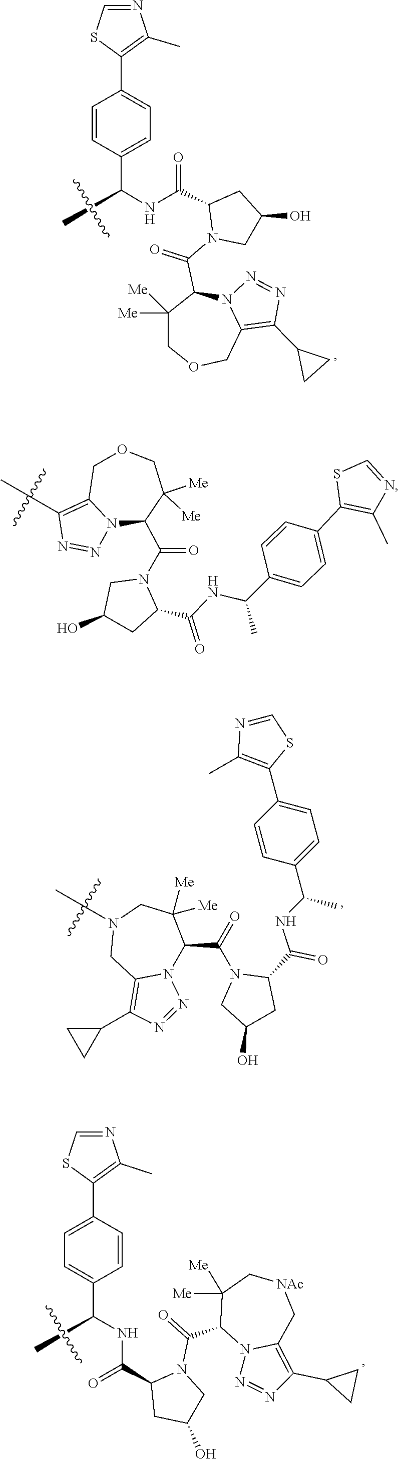

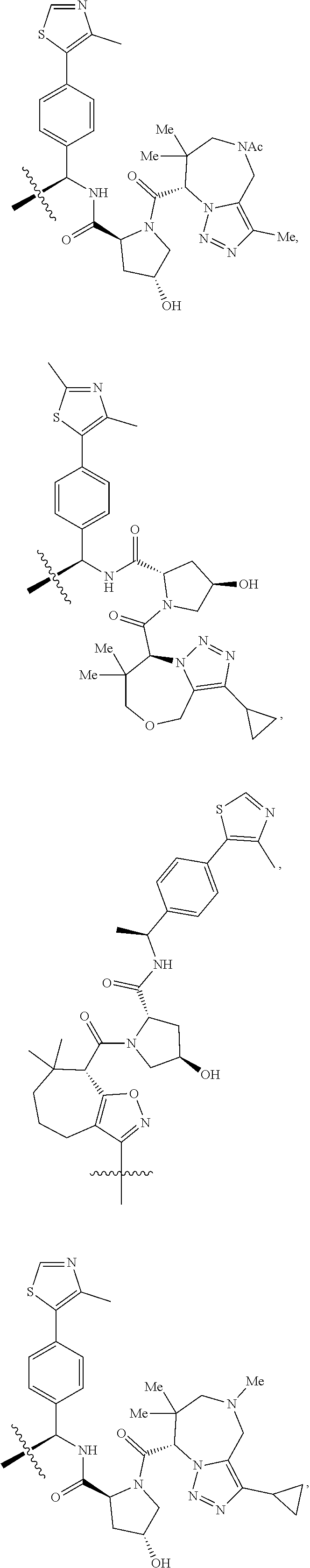

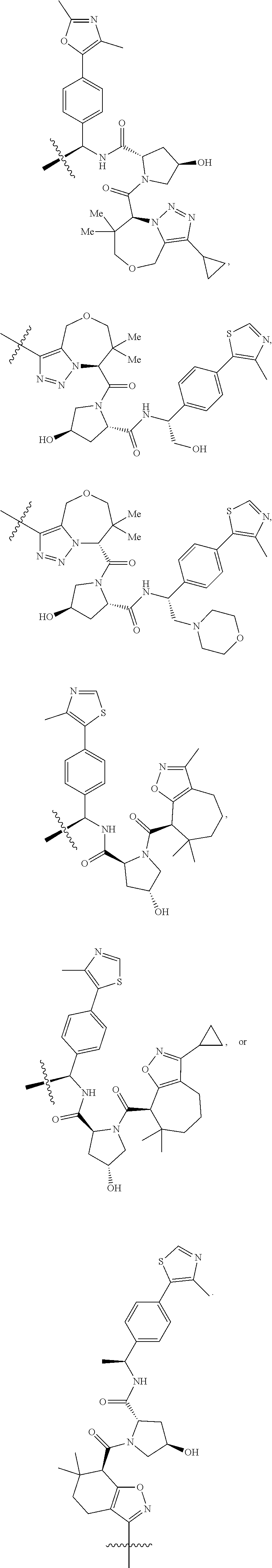

In certain embodiments, the conjugate is of Formula L-XIII:

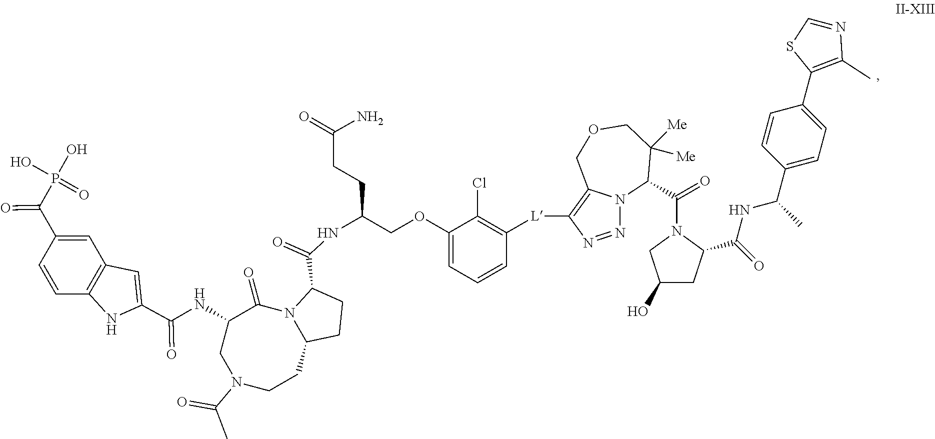

or a pharmaceutically acceptable salt thereof, wherein L′ is C₄₋₆ alkylene,

In certain embodiments, the conjugate is of Formula II-XIV:

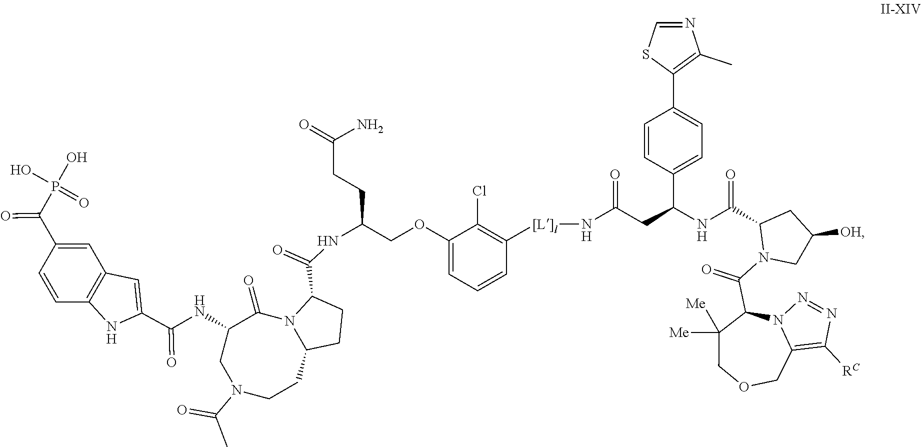

or a pharmaceutically acceptable salt thereof, wherein:
each L′ is independently C₁₋₆ alkylene, —C(═O)—, or —N(R^{L′})—;
R^{L′} is hydrogen;
l is 3 or 4; and
R^{C} is methyl or cyclopropyl

In certain embodiments, the conjugate is B-3, B-8, B-10, or B-18

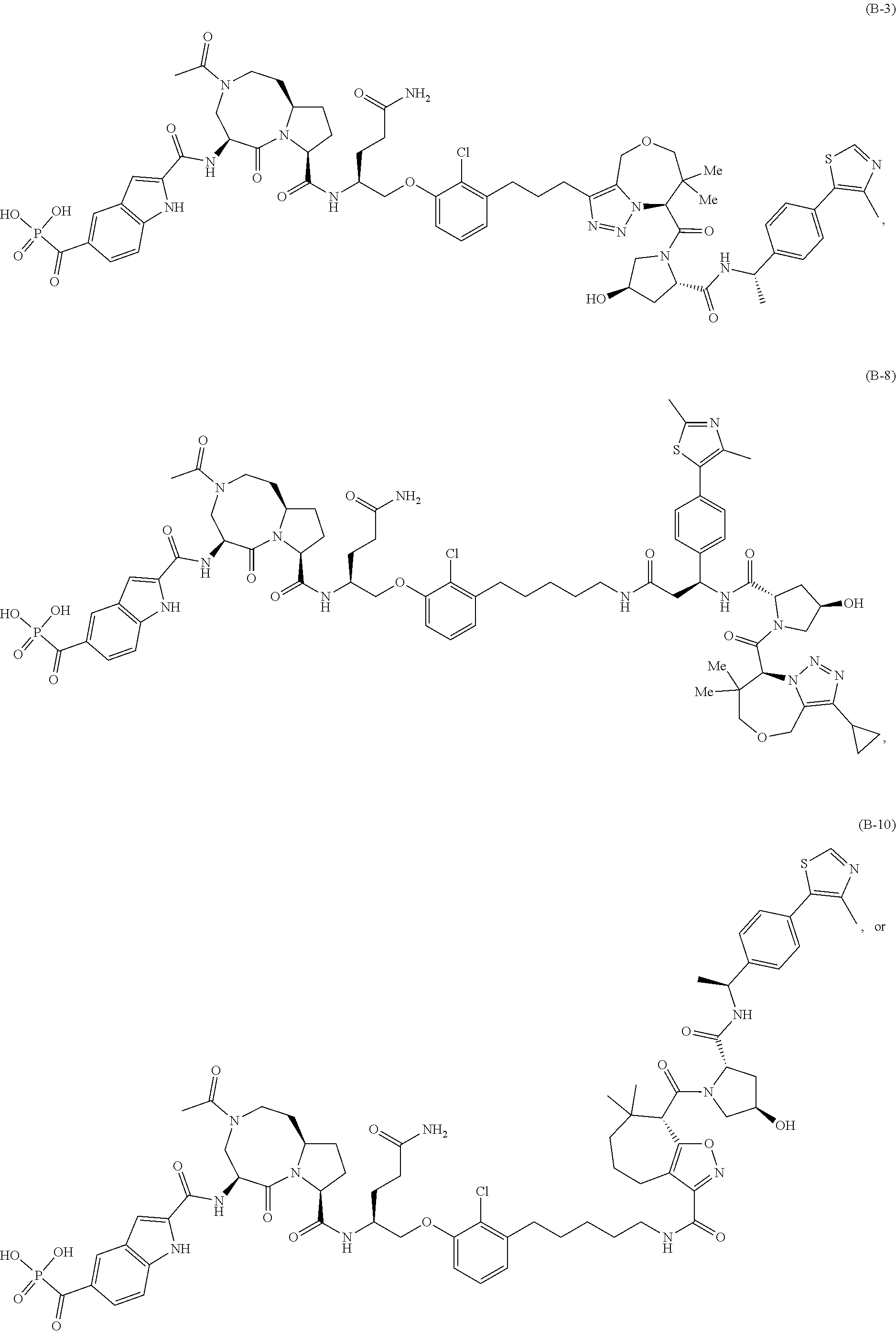

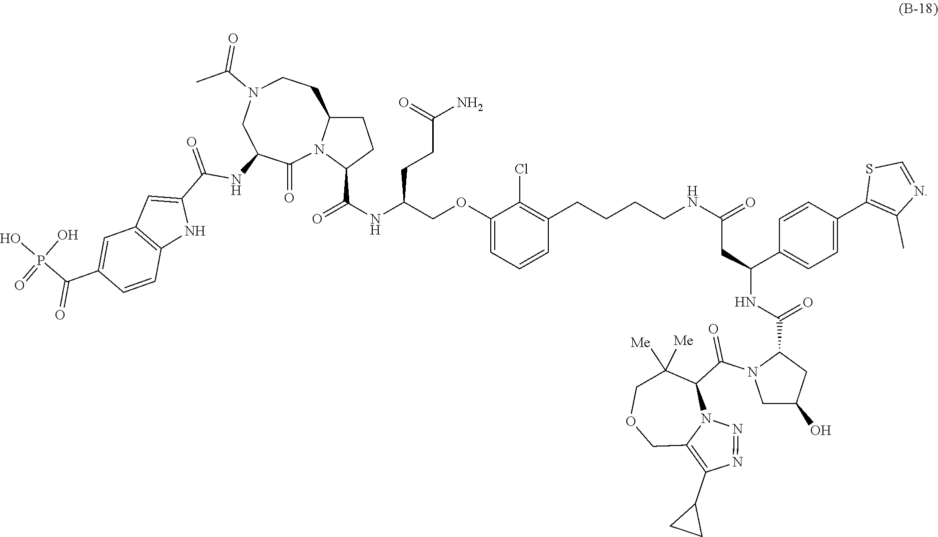 [SMILES: CC(=O)N1CC[C@H]2CC[C@@H](C(=O)N[C@@H](CCC(N)=O)COc3cccc(CCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1]

Embodiments of the variables in any of the Formulae described herein, e.g., Formulae I, I-1-i, I-1-ii, I-1-iii, II-1, II-1-i, II-1-ii, II-1-iii, II-1-iv, II-1-v, II-1-vi, II-1-vii, II-1-viii, II-2, II-3-i-a, II-3-i-b, II-3-i-a, and II-3-ii-b as applicable, are described below. Any of the variables can be any moiety as described in the embodiments below. In addition, the combination of any moieties described for any of the variables, as applicable, with any moieties described for any of the remaining variables, is also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

TABLE 2

| Compound | | |
|---|---|---|
| No | Structure | Chemical Name |
| B1 | 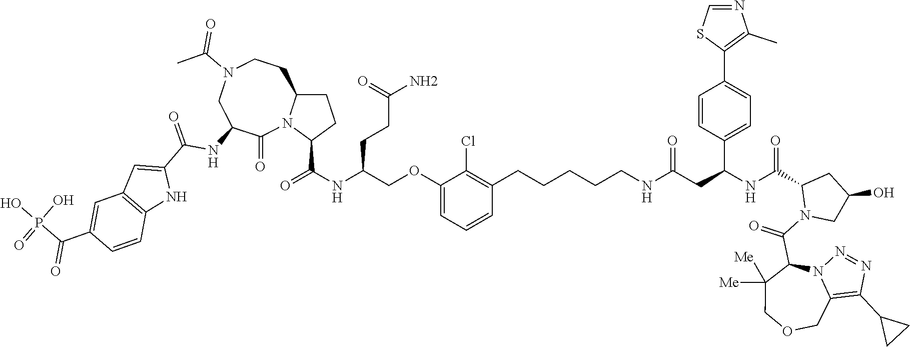 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-((2S,4R)-1-((S)-3- cyclopropyl-7,7-dimethyl-7,8-dihydro-4H,6H- [1,2,3]triazolo[5,1-c][1,4]oxazepine-8-carbon- yl)-4-hydroxypyrrolidine-2-carboxamido)-3- (4-(4-methylthiazol-5-yl)phenyl)propan- amido)pentyl)-phenoxy)-5-oxopentan-2-yl)- carbamoyl)-6-oxodecahydropyrrolo[1,2-a] [1,5]diazocin-5-yl)carbamoyl)-1H-indole-5- carbonyl)phosphonic acid |
| B2 | 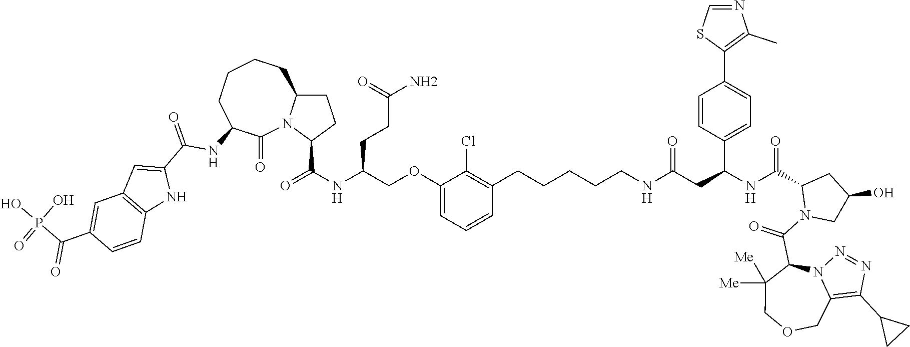 [SMILES: Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)NC(=O)[C@@H]2C[C@@H](O)CN2C(=O)[C@H]2n3nnc(C4CC4)c3COCC2(C)C)cc1] | (2-(((3S,6S,10aS)-3-(((S)-5-amino-1-(2- chloro-3-(5-((S)-3-((2S,4R)-1-((S)-3-cyclo- propyl-7,7-dimethyl-7,8-dihydro-4H,6H- [1,2,3]triazolo[5,1-c][1,4]oxazepine-8-carbon- yl)-4-hydroxypyrrolidine-2-carboxamido)-3- (4-(4-methylthiazol-5-yl)phenyl)propan- amido)pentyl)phenoxy)-5-oxopentan-2-yl)- carbamoyl)-5-oxodecahydropyrrolo[1,2-a] azocin-6-yl)carbamoyl)-1H-indole-5-carbon- yl)phosphonic acid |
| B3 | 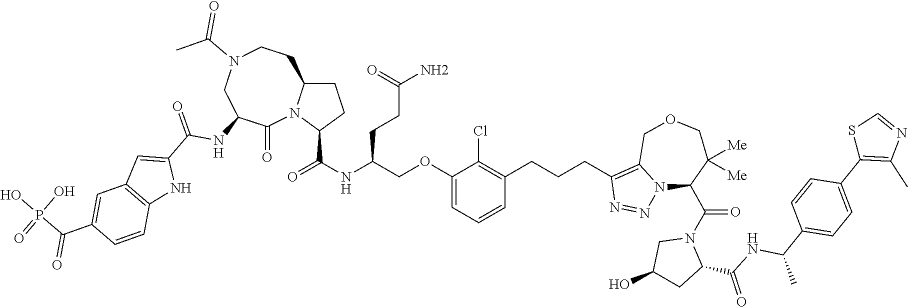 [SMILES: CC(=O)N1CC[C@H]2CC[C@@H](C(=O)N[C@@H](CCC(N)=O)COc3cccc(CCCc4nnn5c4COCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(3-((S)-8-((2S,4R)-4-hydroxy- 2-(((S)-1-(4-(4-methylthiazol-5-yl)phenyl)- ethyl)carbamoyl)pyrrolidine-1-carbonyl)- 7,7-dimethyl-7,8-dihydro-4H,6H-[1,2,3] triazolo[5,1-c][1,4]oxazepin-3-yl)propyl)- phenoxy)-5-oxopentan-2-yl)carbamoyl)-6- oxodecahydropyrrolo[1,2-a][1,5]diazocin-5- yl)carbamoyl)-1H-indole-5-carbonyl)- phosphonic acid |
| B4 | 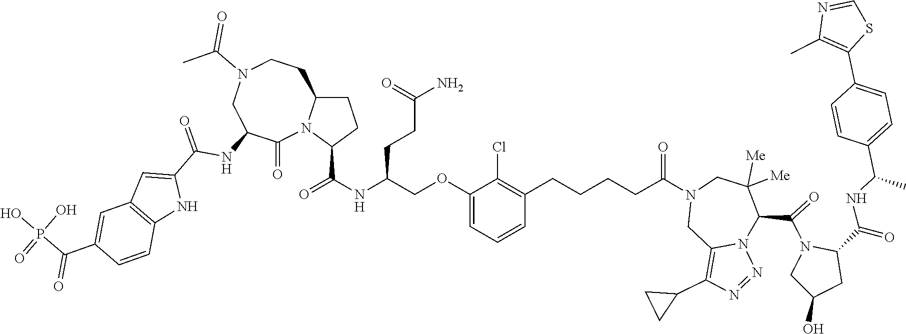 [SMILES: CC(=O)N1CC[C@H]2CC[C@@H](C(=O)N[C@@H](CCC(N)=O)COc3cccc(CCCCC(=O)N4Cc5c(C6CC6)nnn5[C@H](C(=O)N5C[C@H](O)C[C@H]5C(=O)N[C@@H](C)c5ccc(-c6scnc6C)cc5)C(C)(C)C4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-cyclopropyl-8- ((2S,4R)-4-hydroxy-2-(((S)-1-(4-(4-methyl- thiazol-5-yl)phenyl)ethyl)carbamoyl)pyrrol- idine-1-carbonyl)-7,7-dimethyl-7,8-dihydro- 4H-[1,2,3]triazolo[1,5-a][1,4]diazepin-5(6H)- yl)-5-oxopentyl)phenoxy)-5-oxopentan-2-yl)- carbamoyl)-6-oxodecahydropyrrolo[1,2-a] [1,5]diazocin-5-yl)carbamoyl)-1H-indole-5- carbonyl)phosphonic acid |
| B5 | 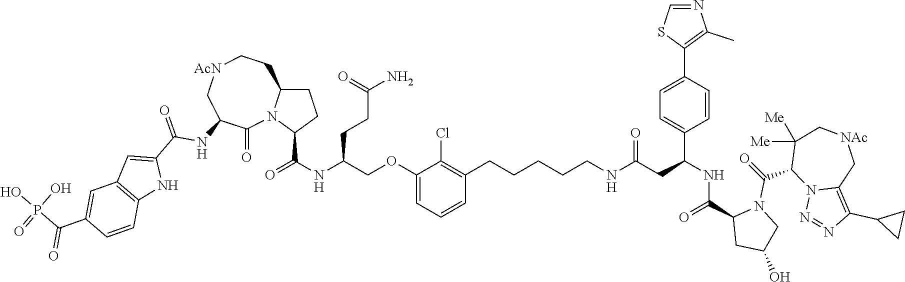 | (2-(((5S,8S,10aR)-3-acetyl-8-(((S)-1-(3-(5- ((S)-3-((2S,4R)-1-((S)-5-acetyl-3-cycloprop- yl-7,7-dimethyl-5,6,7,8-tetrahydro-4H-[1,2,3] triazolo[1,5-a][1,4]diazepine-8-carbonyl)-4- hydroxypyrrolidine-2-carboxamido)-3-(4-(4- methylthiazol-5-yl)phenyl)propanamido)- pentyl)-2-chlorophenoxy)-5-amino-5-oxo- pentan-2-yl)carbamoyl)-6-oxodecahydropyr- rolo[1,2-a][1,5]diazocin-5-yl)carbamoyl)- 1H-indole-5-carbonyl)phosphonic acid |
| B6 | 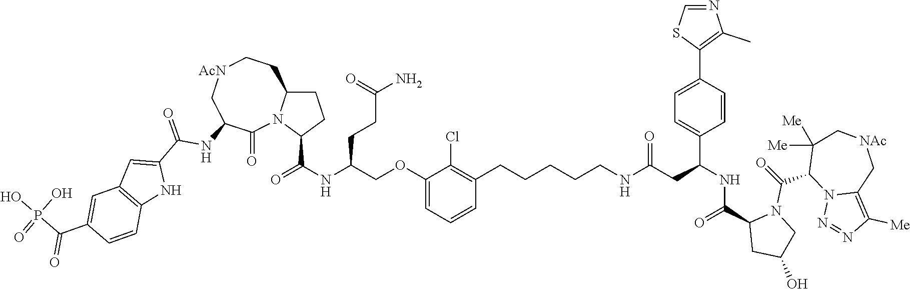 | (2-(((5S,8S,10aR)-3-acetyl-8-(((S)-1-(3-(5- ((S)-3-((2S,4R)-1-((S)-5-acetyl-3,7,7-tri- methyl-5,6,7,8-tetrahydro-4H-[1,2,3]triazolo [1,5-a][1,4]diazepine-8-carbonyl)-4-hydroxy- pyrrolidine-2-carboxamido)-3-(4-(4-methyl- thiazol-5-yl)phenyl)propanamido)pentyl)-2- chlorophenoxy)-5-amino-5-oxopentan-2-yl)- carbamoyl)-6-oxodecahydropyrrolo[1,2-a] [1,5]diazocin-5-yl)carbamoyl)-1H-indole-5- carbonyl)phosphonic acid |
| B7 | 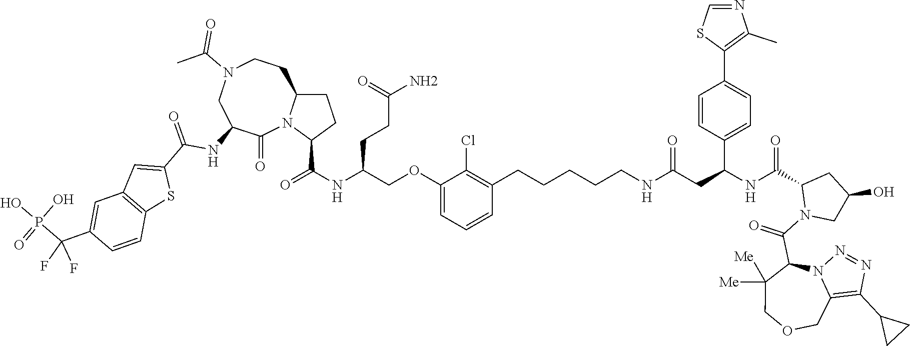 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(F)(F)P(=O)(O)O)ccc3s2)C1] | ((2-(((5S,8S,10aR)-3-acetyl-8-(((S)-5-amino- 1-(2-chloro-3-(5-((S)-3-((2S,4R)-1-((S)-3- cyclopropyl-7,7-dimethyl-7,8-dihydro- 4H,6H-[1,2,3]triazolo[5,1-c][1,4]oxazepine- 8-carbonyl)-4-hydroxypyrrolidine-2-carbox- amido)-3-(4-(4-methylthiazol-5-yl)phenyl)- propanamido)pentyl)phenoxy)-5-oxopentan- 2-yl)carbamoyl)-6-oxodecahydropyrrolo [1,2-a][1,5]diazocin-5-yl)carbamoyl)benzo [b]thiophen-5-yl)difluoromethyl)- phosphonic acid |
| B8 | 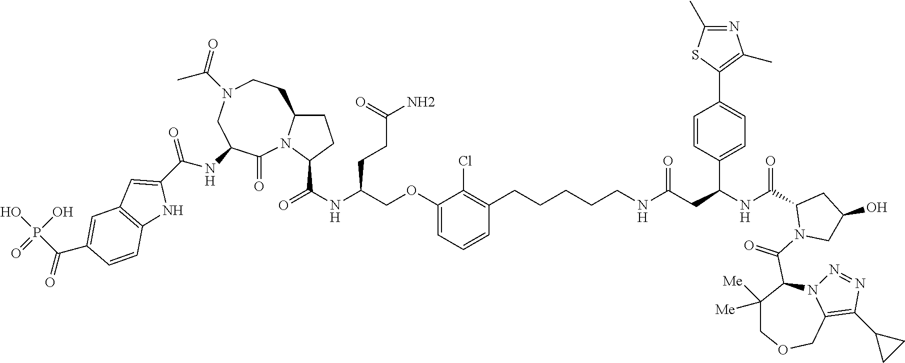 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-((2S,4R)-1-((S)-3- cyclopropyl-7,7-dimethyl-7,8-dihydro- 4H,6H-[1,2,3]triazolo[5,1-c][1,4]oxazepine- 8-carbonyl)-4-hydroxypyrrolidine-2-carbox- amido)-3-(4-(2,4-dimethylthiazol-5-yl)phen- yl)propanamido)pentyl)phenoxy)-5-oxo- pentan-2-yl)carbamoyl)-6-oxodecahydro- pyrrolo[1,2-a][1,5]diazocin-5-yl)carbamoyl)- 1H-indole-5-carbonyl)phosphonic acid |
| B9 | 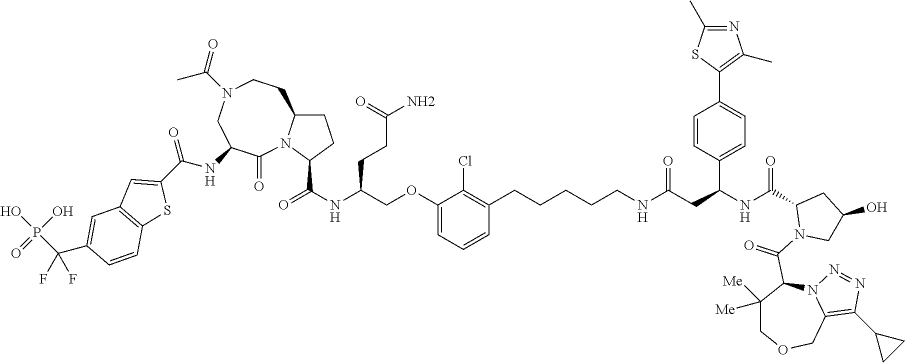 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](NC(=O)c2cc3cc(C(F)(F)P(=O)(O)O)ccc3s2)C1] | ((2-(((5S,8S,10aR)-3-acetyl-8-(((S)-5-amino- 1-(2-chloro-3-(5-((S)-3-((2S,4R)-1-((S)-3- cyclopropyl-7,7-dimethyl-7,8-dihydro- 4H,6H-[1,2,3]triazolo[5,1-c][1,4]oxazepine- 8-carbonyl)-4-hydroxypyrrolidine-2-carbox- amido)-3-(4-(2,4-dimethylthiazol-5-yl)phen- yl)propanamido)pentyl)phenoxy)-5-oxo- pentan-2-yl)carbamoyl)-6-oxodecahydro- pyrrolo[1,2-a][1,5]diazocin-5-yl)carbamoyl)- benzo[b]thiophen-5-yl)difluoromethyl)- phosphonic acid |
| B10 | 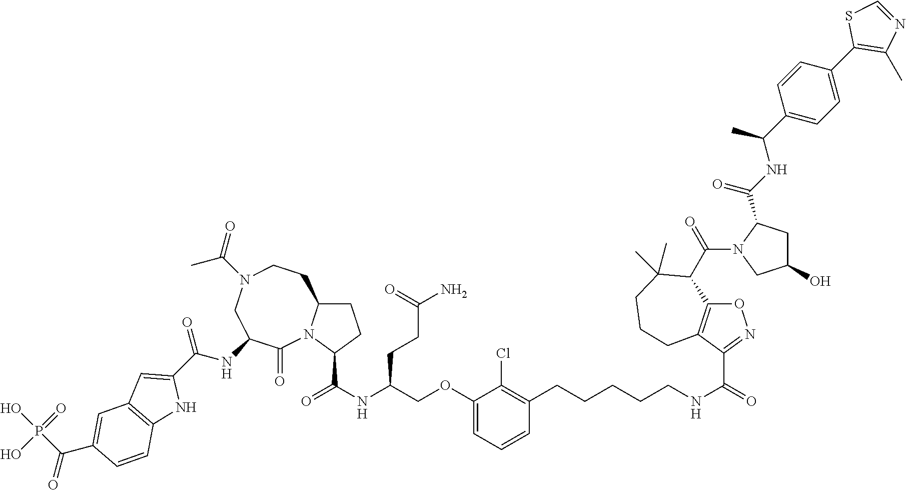 [SMILES: CC(=O)N1CC[C@H]2CC[C@@H](C(=O)N[C@@H](CCC(N)=O)COc3cccc(CCCCCNC(=O)c4noc5c4CCCC(C)(C)[C@@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-8-((2S,4R)-4-hydroxy- 2-(((S)-1-(4-(4-methylthiazol-5-yl)phenyl)- ethyl)carbamoyl)pyrrolidine-1-carbonyl)- 7,7-dimethyl-5,6,7,8-tetrahydro-4H-cyclo- hepta[d]isoxazole-3-carboxamido)pentyl)- phenoxy)-5-oxopentan-2-yl)carbamoyl)-6- oxodecahydropyrrolo[1,2-a][1,5]diazocin-5- yl)carbamoyl)-1H-indole-5-carbonyl)- phosphonic acid |
| B11 | 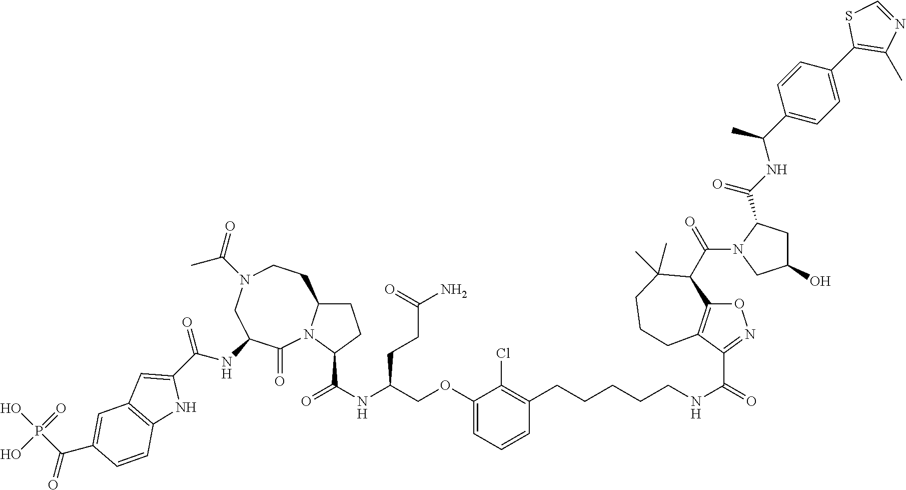 [SMILES: CC(=O)N1CC[C@H]2CC[C@@H](C(=O)N[C@@H](CCC(N)=O)COc3cccc(CCCCCNC(=O)c4noc5c4CCCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((R)-8-((2S,4R)-4-hydroxy- 2-(((S)-1-(4-(4-methylthiazol-5-yl)phenyl)- ethyl)carbamoyl)pyrrolidine-1-carbonyl)- 7,7-dimethyl-5,6,7,8-tetrahydro-4H-cyclo- hepta[d]isoxazole-3-carboxamido)pentyl)- phenoxy)-5-oxopentan-2-yl)carbamoyl)-6- oxodecahydropyrrolo[1,2-a][1,5]diazocin-5- yl)carbamoyl)-1H-indole-5-carbonyl)- phosphonic acid |
| B12 | 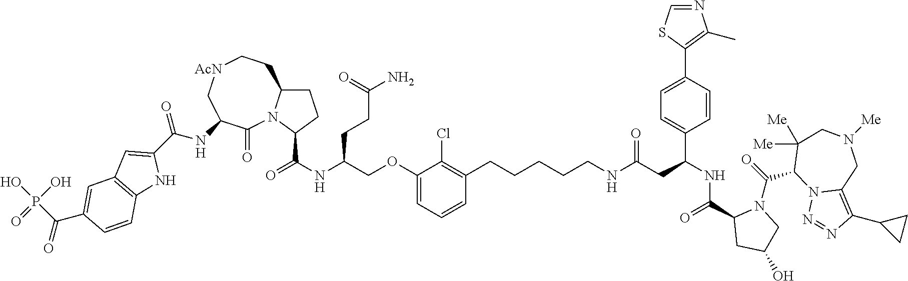 | (2-(((5S,8S,10aR)-3-acetyl-8-(((S)-5-amino- 1-(2-chloro-3-(5-((S)-3-((2S,4R)-1-((S)-3- cyclopropyl-5,7,7-trimethyl-5,6,7,8-tetra- hydro-4H-[1,2,3]triazolo[1,5-a][1,4] diazepine-8-carbonyl)-4-hydroxypyrrolidine- 2-carboxamido)-3-(4-(4-methylthiazol-5- yl)phenyl)propanamido)pentyl)phenoxy)-5- oxopentan-2-yl)carbamoyl)-6-oxodecahydro- pyrrolo[1,2-a][1,5]diazocin-5-yl)carbamoyl)- 1H-indole-5-carbonyl)phosphonic acid |
| B13 | 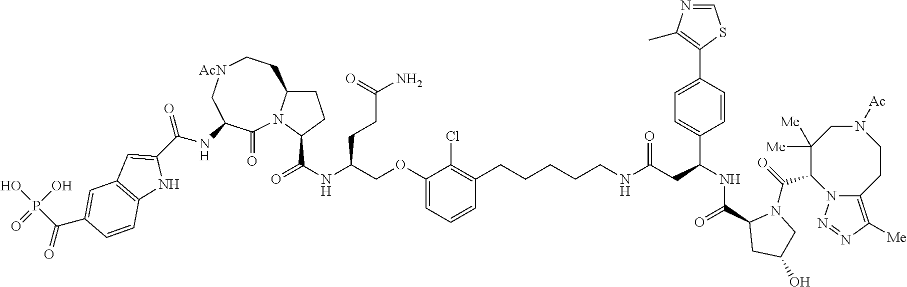 | (2-(((5S,8S,10aR)-3-acetyl-8-(((S)-1-(3-(5- ((S)-3-((2S,4R)-1-((S)-6-acetyl-3,8,8- trimethyl-4,5,6,7,8,9-hexahydro-[1,2,3] triazolo[1,5-a][1,5]diazocine-9-carbonyl)-4- hydroxypyrrolidine-2-carboxamido)-3-(4-(4- methylthiazol-5-yl)phenyl)propanamido)- pentyl)-2-chlorophenoxy)-5-amino-5-oxo- pentan-2-yl)carbamoyl)-6-oxodecahydro- pyrrolo[1,2-a][1,5]diazocin-5-yl)carbamoyl)- 1H-indole-5-carbonyl)phosphonic acid |
| B14 | 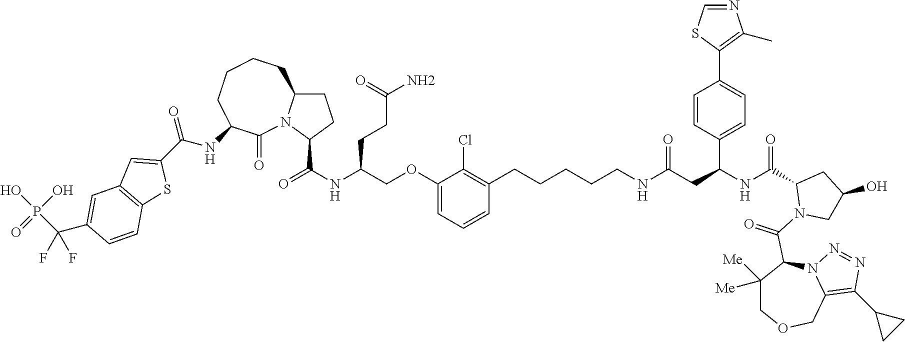 [SMILES: Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(F)(F)P(=O)(O)O)ccc6s5)C(=O)N43)c2Cl)NC(=O)[C@@H]2C[C@@H](O)CN2C(=O)[C@H]2n3nnc(C4CC4)c3COCC2(C)C)cc1] | ((2-(((3S,6S,10aS)-3-(((S)-5-amino-1-(2- chloro-3-(5-((S)-3-((2S,4R)-1-((S)-3-cyclo- propyl-7,7-dimethyl-7,8-dihydro-4H,6H- [1,2,3]triazolo[5,1-c][1,4]oxazepine-8- carbonyl)-4-hydroxypyrrolidine-2-carbox- amido)-3-(4-(4-methylthiazol-5-yl)phenyl)- propanamido)pentyl)phenoxy)-5-oxopentan- 2-yl)carbamoyl)-5-oxodecahydropyrrolo[1,2- a]azocin-6-yl)carbamoyl)benzo[b]thiophen- 5-yl)difluoromethyl)phosphonic acid |
| B15 | 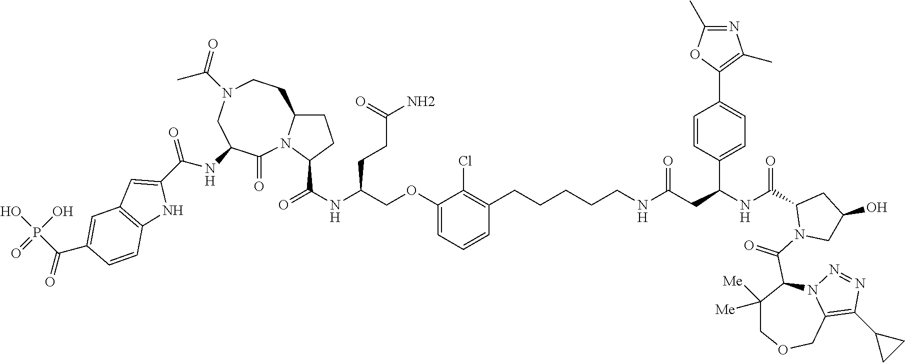 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5oc(C)nc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-((2S,4R)-1-((S)-3- cyclopropyl-7,7-dimethyl-7,8-dihydro- 4H,6H-[1,2,3]triazolo[5,1-c][1,4]oxazepine- 8-carbonyl)-4-hydroxypyrrolidine-2- carboxamido)-3-(4-(2,4-dimethyloxazol-5- yl)phenyl)propanamido)pentyl)phenoxy)-5- oxopentan-2-yl)carbamoyl)-6-oxodecahydro- pyrrolo[1,2-a][1,5]diazocin-5-yl)carbamoyl)- 1H-indole-5-carbonyl)phosphonic acid |
| B16 | 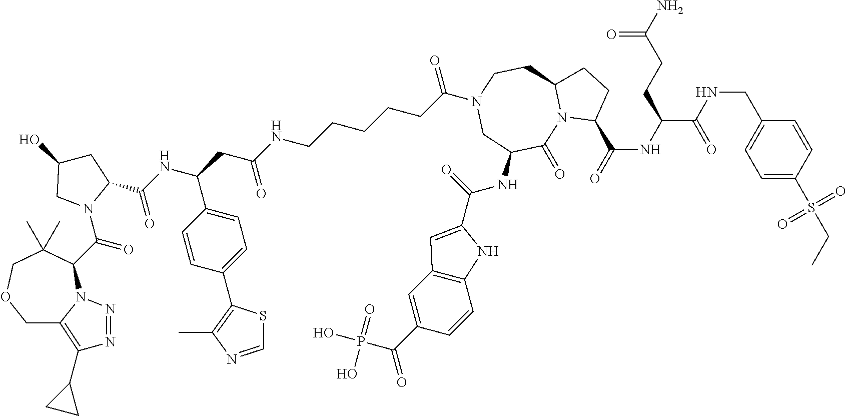 [SMILES: CCS(=O)(=O)c1ccc(CNC(=O)[C@H](CCC(N)=O)NC(=O)[C@@H]2CC[C@@H]3CCN(C(=O)CCCCCNC(=O)C[C@H](NC(=O)[C@H]4C[C@H](O)CN4C(=O)[C@H]4n5nnc(C6CC6)c5COCC4(C)C)c4ccc(-c5scnc5C)cc4)C[C@H](NC(=O)c4cc5cc(C(=O)P(=O)(O)O)ccc5[nH]4)C(=O)N32)cc1] | (2-(((5S,8S,10aR)-8-(((S)-5-amino-1-((4- (ethylsulfonyl)benzyl)amino)-1,5-dioxo- pentan-2-yl)carbamoyl)-3-(6-((S)-3-((2R,4S)- 1-((S)-3-cyclopropyl-7,7-dimethyl-7,8- dihydro-4H,6H-[1,2,3]triazolo[5,1-c][1,4] oxazepine-8-carbonyl)-4-hydroxypyrrol- idine-2-carboxamido)-3-(4-(4-methylthiazol- 5-yl)phenyl)propanamido)hexanoyl)-6-oxo- decahydropyrrolo[1,2-a][1,5]diazocin-5-yl)- carbamoyl)-1H-indole-5-carbonyl)- phosphonic acid |
| B17 | 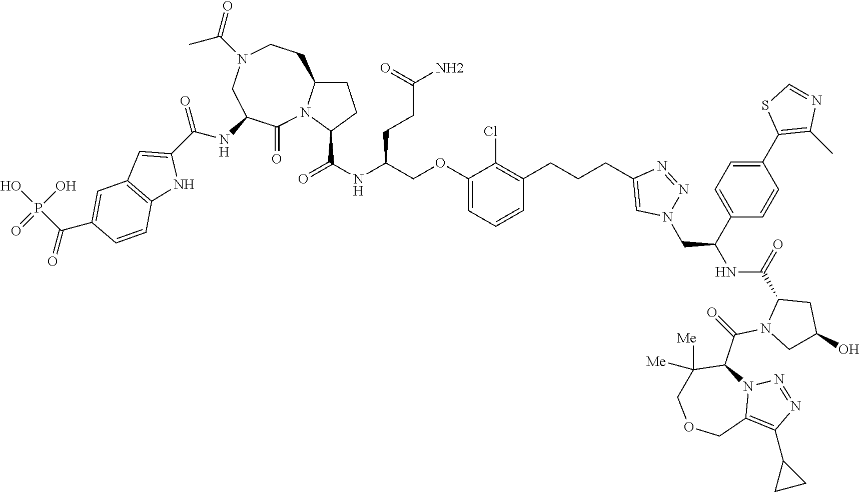 [SMILES: CC(=O)N1CC[C@H]2CC[C@@H](C(=O)N[C@@H](CCC(N)=O)COc3cccc(CCCc4cn(C[C@H](NC(=O)[C@@H]5C[C@@H](O)CN5C(=O)[C@H]5n6nnc(C7CC7)c6COCC5(C)C)c5ccc(-c6scnc6C)cc5)nn4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(3-(1-((R)-2-((2S,4R)-1-((S)- 3-cyclopropyl-7,7-dimethyl-7,8-dihydro- 4H,6H-[1,2,3]triazolo[5,1-c][1,4]oxazepine- 8-carbonyl)-4-hydroxypyrrolidine-2-carbox- amido)-2-(4-(4-methylthiazol-5-yl)phenyl)- ethyl)-1H-1,2,3-triazol-4-yl)propyl)phen- oxy)-5-oxopentan-2-yl)carbamoyl)-6-oxo- decahydropyrrolo[1,2-a][1,5]diazocin-5- yl)carbamoyl)-1H-indole-5-carbonyl)- phosphonic acid |
| B18 | 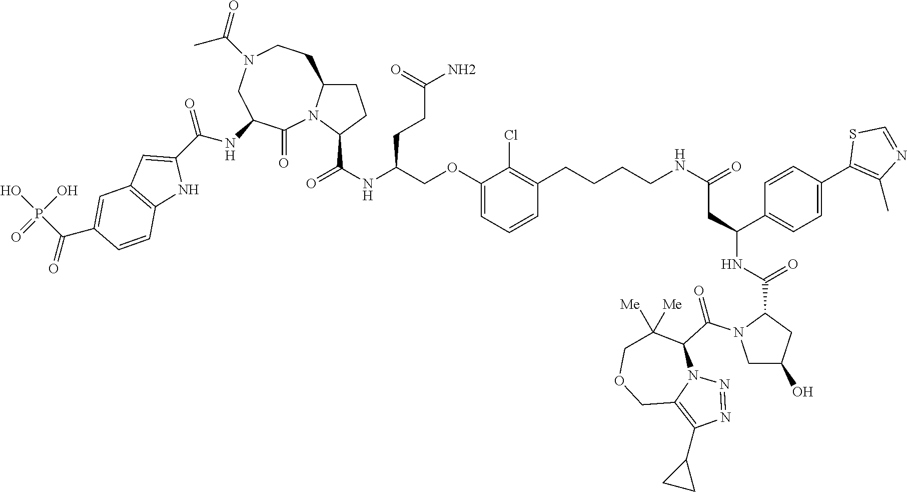 [SMILES: CC(=O)N1CC[C@H]2CC[C@@H](C(=O)N[C@@H](CCC(N)=O)COc3cccc(CCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(4-((S)-3-((2S,4R)-1-((S)-3- cyclopropyl-7,7-dimethyl-7,8-dihydro- 4H,6H-[1,2,3]triazolo[5,1-c][1,4]oxazepine- 8-carbonyl)-4-hydroxypyrrolidine-2-carbox- amido)-3-(4-(4-methylthiazol-5-yl)phenyl)- propanamido)butyl)phenoxy)-5-oxopentan- 2-yl)carbamoyl)-6-oxodecahydropyrrolo [1,2-a][1,5]diazocin-5-yl)carbamoyl)-1H- indole-5-carbonyl)phosphonic acid |
| B19 | 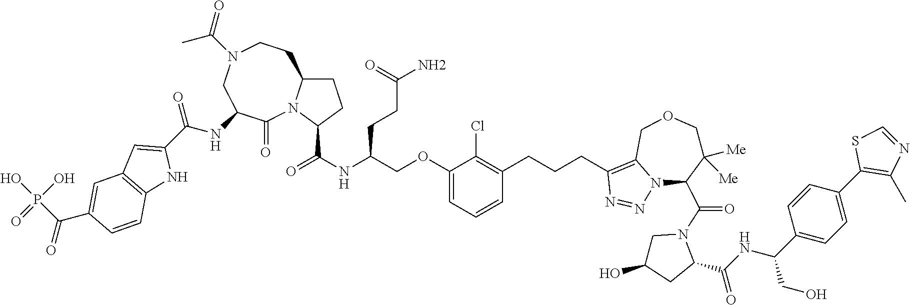 [SMILES: CC(=O)N1CC[C@H]2CC[C@@H](C(=O)N[C@@H](CCC(N)=O)COc3cccc(CCCc4nnn5c4COCC(C)(C)[C@H]5C(=O)N4C[C@H](O)C[C@H]4C(=O)N[C@@H](CO)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(3-((S)-8-((2S,4R)-4-hydroxy- 2-(((R)-2-hydroxy-1-(4-(4-methylthiazol-5- yl)phenyl)ethyl)carbamoyl)pyrrolidine-1- carbonyl)-7,7-dimethyl-7,8-dihydro-4H,6H- [1,2,3]triazolo[5,1-c][1,4]oxazepin-3-yl)- propyl)phenoxy)-5-oxopentan-2-yl)- carbamoyl)-6-oxodecahydropyrrolo[1,2-a] [1,5]diazocin-5-yl)carbamoyl)-1H-indole-5- carbonyl)phosphonic acid |
| B20 | 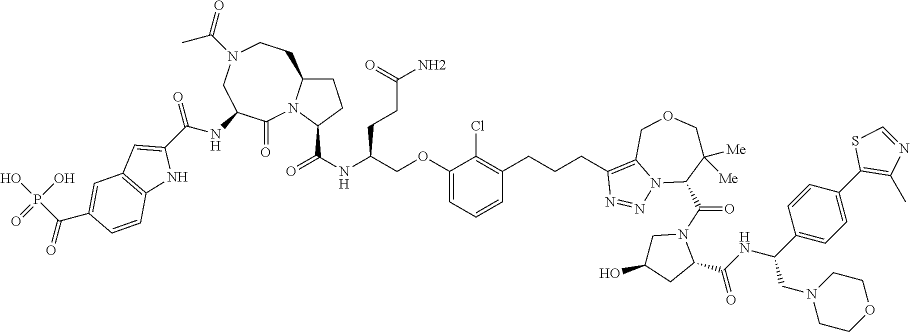 [SMILES: CC(=O)N1CC[C@H]2CC[C@@H](C(=O)N[C@@H](CCC(N)=O)COc3cccc(CCCc4nnn5c4COCC(C)(C)[C@@H]5C(=O)N4C[C@H](O)C[C@H]4C(=O)N[C@@H](CN4CCOCC4)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(3-((S)-8-((2S,4R)-4-hydroxy- 2-(((R)-1-(4-(4-methylthiazol-5-yl)phenyl)- 2-morpholinoethyl)carbamoyl)pyrrolidine-1- carbonyl)-7,7-dimethyl-7,8-dihydro-4H,6H- [1,2,3]triazolo[5,1-c][1,4]oxazepin-3-yl)- propyl)phenoxy)-5-oxopentan-2-yl)carbamo- yl)-6-oxodecahydropyrrolo[1,2-a][1,5] diazocin-5-yl)carbamoyl)-1H-indole-5- carbonyl)phosphonic acid |
| B21 | 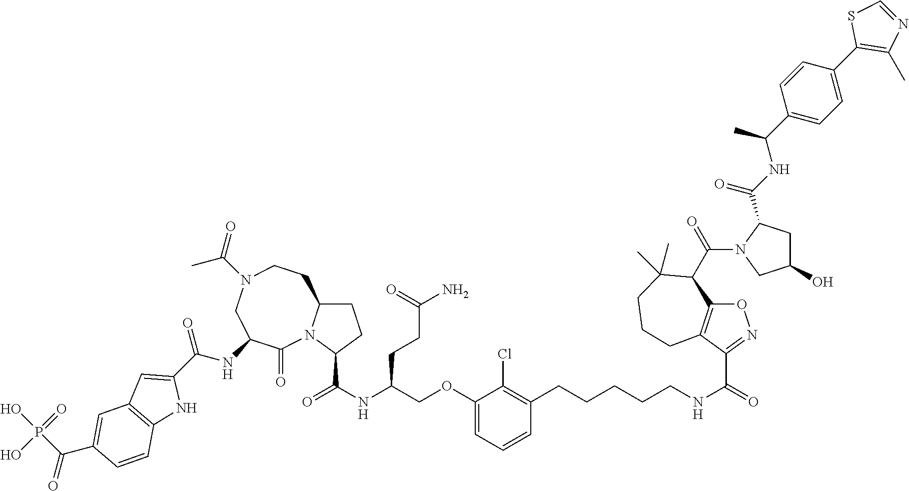 [SMILES: CC(=O)N1CC[C@H]2CC[C@@H](C(=O)N[C@@H](CCC(N)=O)COc3cccc(CCCCCNC(=O)c4noc5c4CCCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((R)-8-((2S,4R)-4-hydroxy- 2-(((S)-1-(4-(4-methylthiazol-5-yl)phenyl)- ethyl)carbamoyl)pyrrolidine-1-carbonyl)-7,7- dimethyl-5,6,7,8-tetrahydro-4H-cyclohepta [d]isoxazole-3-carboxamido)pentyl)phen- oxy)-5-oxopentan-2-yl)carbamoyl)-6-oxo- decahydropyrrolo[1,2-a][1,5]diazocin-5-yl)- carbamoyl)-1H-indole-5-carbonyl)- phosphonic acid |
| B22 | 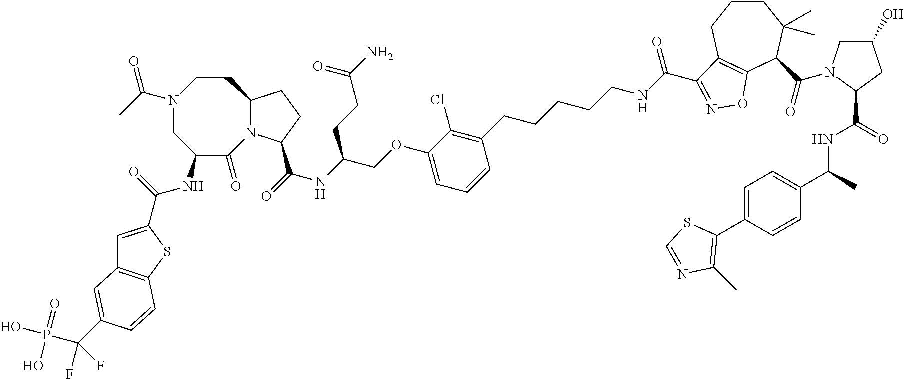 [SMILES: CC(=O)N1CC[C@H]2CC[C@@H](C(=O)N[C@@H](CCC(N)=O)COc3cccc(CCCCCNC(=O)c4noc5c4CCCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(F)(F)P(=O)(O)O)ccc3s2)C1] | ((2-(((5S,8S,10aR)-3-acetyl-8-(((S)-5-amino- 1-(2-chloro-3-(5-((R)-8-((2S,4R)-4-hydroxy- 2-(((S)-1-(4-(4-methylthiazol-5-yl)phenyl)- ethyl)carbamoyl)pyrrolidine-1-carbonyl)-7,7- dimethyl-5,6,7,8-tetrahydro-4H-cyclohepta [d]isoxazole-3-carboxamido)pentyl)phen- oxy)-5-oxopentan-2-yl)carbamoyl)-6-oxo- decahydropyrrolo[1,2-a][1,5]diazocin-5-yl)- carbamoyl)benzo[b]thiophen-5-yl)difluoro- methyl)phosphonic acid |
| B23 | 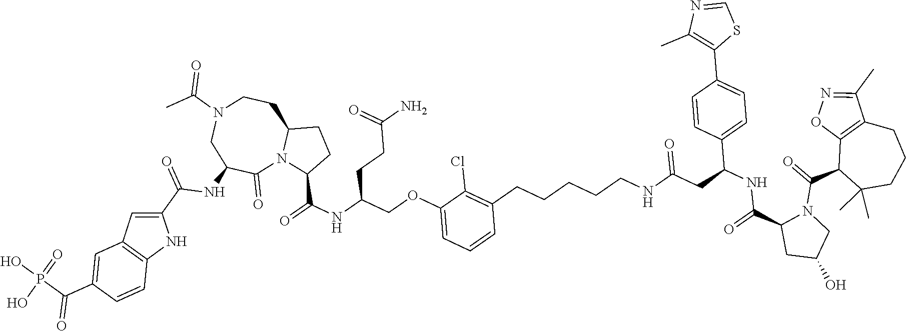 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4c5onc(C)c5CCCC4(C)C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-((2S,4R)-4-hydroxy- 1-((R)-3,7,7-trimethyl-5,6,7,8-tetrahydro-4H- cyclohepta[d]isoxazole-8-carbonyl)pyrrol- idine-2-carboxamido)-3-(4-(4-methylthiazol- 5-yl)phenyl)propanamido)pentyl)phenoxy)- 5-oxopentan-2-yl)carbamoyl)-6-oxodeca- hydropyrrolo[1,2-a][1,5]diazocin-5-yl)- carbamoyl)-1H-indole-5-carbonyl)- phosphonic acid |
| B24 | 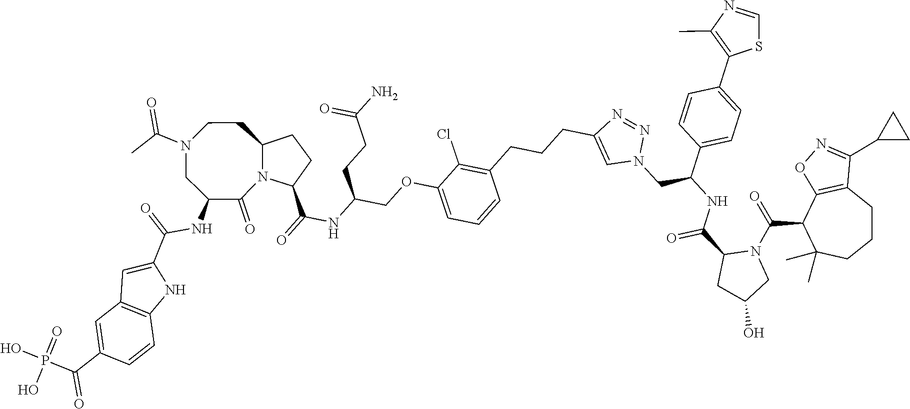 [SMILES: CC(=O)N1CC[C@H]2CC[C@@H](C(=O)N[C@@H](CCC(N)=O)COc3cccc(CCCc4cn(C[C@H](NC(=O)[C@@H]5C[C@@H](O)CN5C(=O)[C@H]5c6onc(C7CC7)c6CCCC5(C)C)c5ccc(-c6scnc6C)cc5)nn4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(3-(1-((R)-2-((2S,4R)-1-((R)- 3-cyclopropyl-7,7-dimethyl-5,6,7,8-tetra- hydro-4H-cyclohepta[d]isoxazole-8-carbon- yl)-4-hydroxypyrrolidine-2-carboxamido)-2- (4-(4-methylthiazol-5-yl)phenyl)ethyl)-1H- 1,2,3-triazol-4-yl)propyl)phenoxy)-5-oxo- pentan-2-yl)carbamoyl)-6-oxodecahydro- pyrrolo[1,2-a][1,5]diazocin-5-yl)carbamo- yl)-1H-indole-5-carbonyl)phosphonic acid |
| B25 | 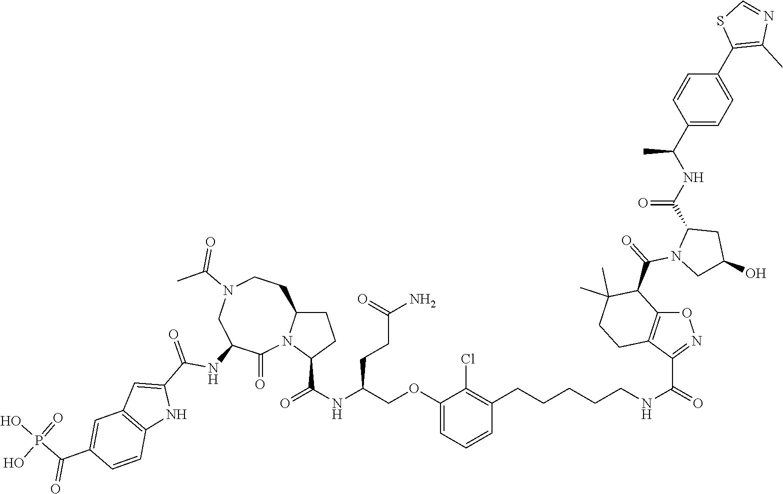 [SMILES: CC(=O)N1CC[C@H]2CC[C@@H](C(=O)N[C@@H](CCC(N)=O)COc3cccc(CCCCCNC(=O)c4noc5c4CCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((R)-7-((2S,4R)-4-hydroxy- 2-(((S)-1-(4-(4-methylthiazol-5-yl)phenyl)- ethyl)carbamoyl)pyrrolidine-1-carbonyl)- 6,6-dimethyl-4,5,6,7-tetrahydrobenzo[d] isoxazole-3-carboxamido)pentyl)phenoxy)- 5-oxopentan-2-yl)carbamoyl)-6-oxodeca- hydropyrrolo[1,2-a][1,5]diazocin-5-yl)- carbamoyl)-1H-indole-5-carbonyl)- phosphonic acid |
| B26 | 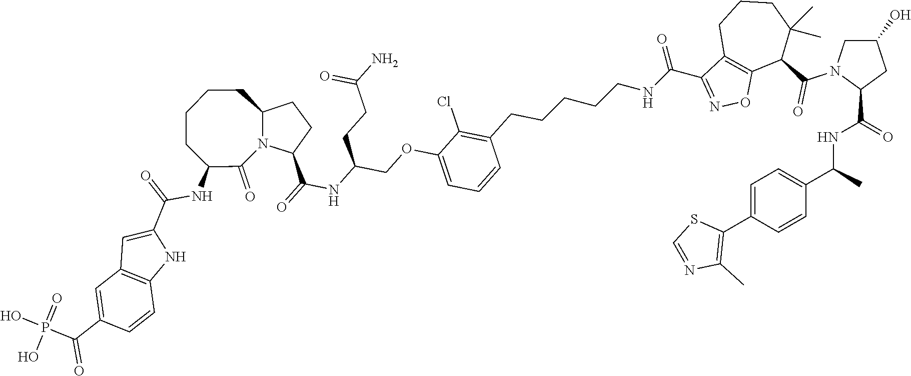 [SMILES: Cc1ncsc1-c1ccc([C@H](C)NC(=O)[C@@H]2C[C@@H](O)CN2C(=O)[C@H]2c3onc(C(=O)NCCCCCc4cccc(OC[C@H](CCC(N)=O)NC(=O)[C@@H]5CC[C@@H]6CCCC[C@H](NC(=O)c7cc8cc(C(=O)P(=O)(O)O)ccc8[nH]7)C(=O)N65)c4Cl)c3CCCC2(C)C)cc1] | (2-(((3S,6S,10aS)-3-(((S)-5-amino-1-(2- chloro-3-(5-((R)-8-((2S,4R)-4-hydroxy-2- (((S)-1-(4-(4-methylthiazol-5-yl)phenyl)- ethyl)carbamoyl)pyrrolidine-1-carbonyl)- 7,7-dimethyl-5,6,7,8-tetrahydro-4H-cyclo- hepta[d]isoxazole-3-carboxamido)pentyl)- phenoxy)-5-oxopentan-2-yl)carbamoyl)-5- oxodecahydropyrrolo[1,2-a]azocin-6-yl)- carbamoyl)-1H-indole-5-carbonyl)- phosphonic acid |
| B27 | 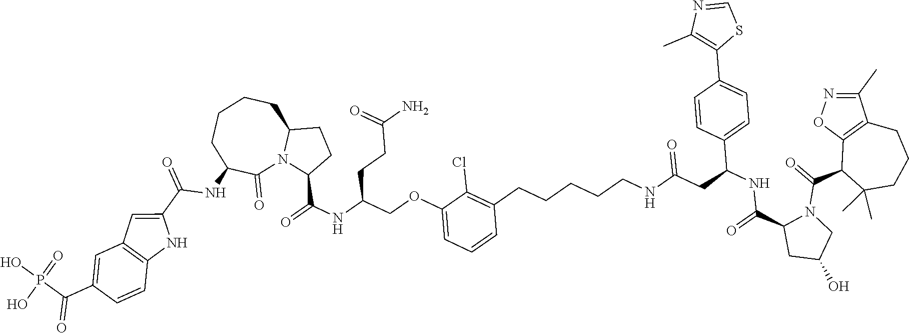 [SMILES: Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)NC(=O)[C@@H]2C[C@@H](O)CN2C(=O)[C@H]2c3onc(C)c3CCCC2(C)C)cc1] | (2-(((3S,6S,10aS)-3-(((S)-5-amino-1-(2- chloro-3-(5-((S)-3-((2S,4R)-4-hydroxy-1- ((R)-3,7,7-trimethyl-5,6,7,8-tetrahydro-4H- cyclohepta[d]isoxazole-8-carbonyl)pyrrol- idine-2-carboxamido)-3-(4-(4-methylthiazol- 5-yl)phenyl)propanamido)pentyl)phenoxy)- 5-oxopentan-2-yl)carbamoyl)-5-oxodeca- hydropyrrolo[1,2-a]azocin-6-yl)carbamoyl)- 1H-indole-5-carbonyl)phosphonic acid |
| B28 | 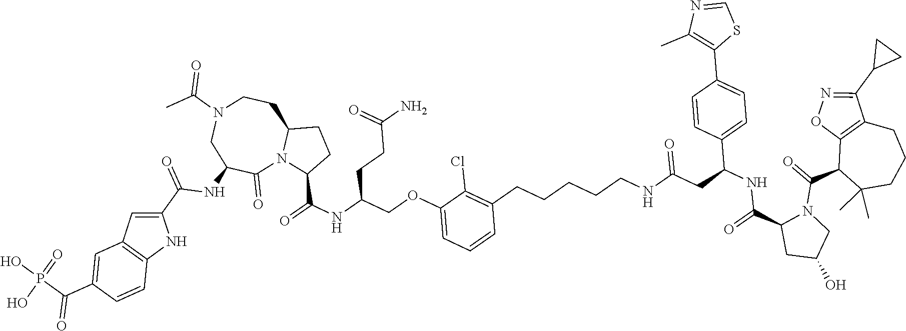 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4c5onc(C6CC6)c5CCCC4(C)C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(5-((S)-3-((2S,4R)-1-((R)-3- cyclopropyl-7,7-dimethyl-5,6,7,8-tetrahydro- 4H-cyclohepta[d]isoxazole-8-carbonyl)-4- hydroxypyrrolidine-2-carboxamido)-3-(4- (4-methylthiazol-5-yl)phenyl)propanamido)- pentyl)phenoxy)-5-oxopentan-2-yl)carbamo- yl)-6-oxodecahydropyrrolo[1,2-a][1,5] diazocin-5-yl)carbamoyl)-1H-indole-5- carbonyl)phosphonic acid |
| B29 | 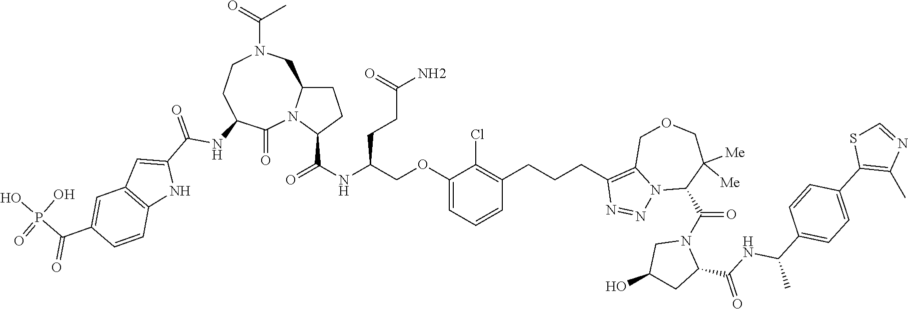 [SMILES: CC(=O)N1CC[C@H](NC(=O)c2cc3cc(C(=O)P(=O)(O)O)ccc3[nH]2)C(=O)N2[C@H](CC[C@H]2C(=O)N[C@@H](CCC(N)=O)COc2cccc(CCCc3nnn4c3COCC(C)(C)[C@@H]4C(=O)N3C[C@H](O)C[C@H]3C(=O)N[C@@H](C)c3ccc(-c4scnc4C)cc3)c2Cl)C1] | (2-(((5S,8S,10aR)-2-acetyl-8-(((S)-5-amino- 1-(2-chloro-3-(3-((R)-8-((2S,4R)-4-hydroxy- 2-(((S)-1-(4-(4-methylthiazol-5-yl)phenyl)- ethyl)carbamoyl)pyrrolidine-1-carbonyl)- 7,7-dimethyl-7,8-dihydro-4H,6H-[1,2,3] triazolo[5,1-c][1,4]oxazepin-3-yl)propyl)- phenoxy)-5-oxopentan-2-yl)carbamoyl)-6- oxodecahydropyrrolo[1,2-a][1,4]diazocin-5- yl)carbamoyl)-1H-indole-5-carbonyl)- phosphonic acid |
| B30 | 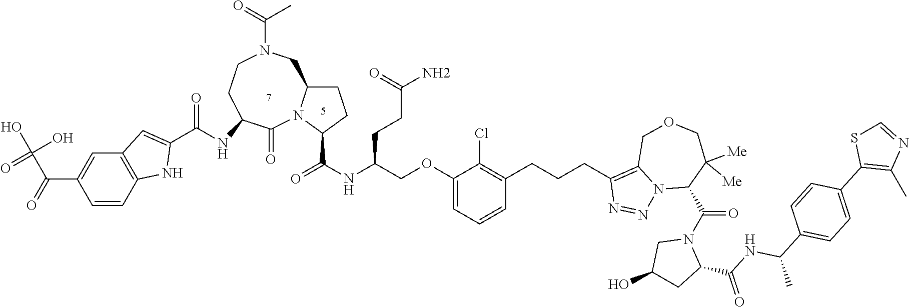 | (2-(((4S,7S,9aR)-2-acetyl-7-(((S)-5-amino-1- (2-chloro-3-(3-((R)-8-((2S,4R)-4-hydroxy-2- (((S)-1-(4-(4-methylthiazol-5-yl)phenyl)- ethyl)carbamoyl)pyrrolidine-1-carbonyl)- 7,7-dimethyl-7,8-dihydro-4H,6H-[1,2,3] triazolo[5,1-c][1,4]oxazepin-3-yl)propyl)- phenoxy)-5-oxopentan-2-yl)carbamoyl)-5- oxooctahydro-1H-pyrrolo[1,2-a][1,4] diazepin-4-yl)carbamoyl)-1H-indole-5- carbonyl)phosphonic acid |
| B31 | 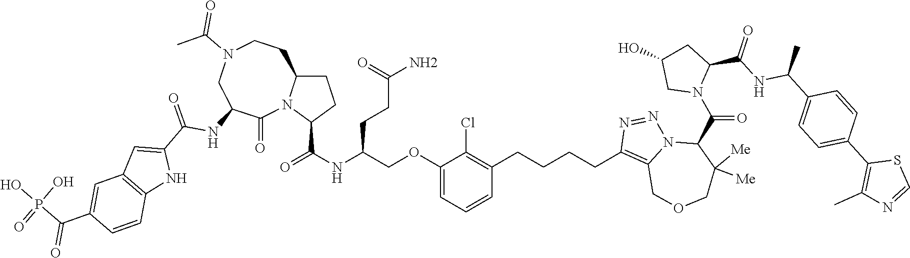 [SMILES: CC(=O)N1CC[C@H]2CC[C@@H](C(=O)N[C@@H](CCC(N)=O)COc3cccc(CCCCc4nnn5c4COCC(C)(C)[C@@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] | (2-(((5S,8S,10aR)-3-acetyl-8-(((S)-5-amino- 1-(2-chloro-3-(4-((R)-8-((2S,4R)-4-hydroxy- 2-(((S)-1-(4-(4-methylthiazol-5-yl)phenyl)- ethyl)carbamoyl)pyrrolidine-1-carbonyl)- 7,7-dimethyl-7,8-dihydro-4H,6H-[1,2,3] triazolo[5,1-c][1,4]oxazepin-3-yl)butyl)- phenoxy)-5-oxopentan-2-yl)carbamoyl)- 6-oxodecahydropyrrolo[1,2-a][1,5]diazocin- 5-yl)carbamoyl)-1H-indole-5-carbonyl)- phosphonic acid |

The compounds or conjugates of the present disclosure may possess advantageous characteristics, as compared to known compounds or conjugates, such as known VHL-binding agents or known degraders comprising such VHL-binding agents. For example, the compounds or conjugates of the present disclosure may display more potent VHL-binding activity or more potent degradation activity against certain proteins (e.g., STAT3), more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds or conjugates of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N*(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight (S)-compound, at least about 99% by weight (R)-compound and at most about 1% by weight (S)-compound, or at least about 99.9% by weight (R)-compound and at most about 0.1% by weight (S)-compound. In certain embodiments, the weights are based upon total weight of compound.

As used herein and unless otherwise indicated, the term “enantiomerically pure (S)-compound” refers to at least about 95% by weight (S)-compound and at most about 5% by weight (R)-compound, at least about 99% by weight (S)-compound and at most about 1% by weight (R)-compound or at least about 99.9% by weight (S)-compound and at most about 0.1% by weight (R)-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In certain embodiments, the enantiomerically pure (R)-compound in such compositions can, for example, comprise, at least about 95% by weight (R)-compound and at most about 5% by weight (S)-compound, by total weight of the compound. For example, a pharmaceutical composition comprising enantiomerically pure (S)-compound can comprise, for example, about 90% excipient and about 10% enantiomerically pure (S)-compound. In certain embodiments, the enantiomerically pure (S)-compound in such compositions can, for example, comprise, at least about 95% by weight (S)-compound and at most about 5% by weight (R)-compound, by total weight of the compound. In certain embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Pharmaceutical Compositions

In certain embodiments, the compound or conjugate described herein is administered as a pure chemical. In certain embodiments, the compound or conjugate described herein is combined with one or more pharmaceutically suitable or acceptable carriers (also referred to herein as one or more pharmaceutically suitable (or acceptable) excipients, physiologically suitable (or acceptable) excipients, or physiologically suitable (or acceptable) carriers) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound or conjugate described herein, or a pharmaceutically acceptable salt, solvate, or stereoisomer thereof, and a pharmaceutically acceptable excipient.

In certain embodiments, the compound or conjugate provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka) (Pittsburgh, PA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Advanced Organic Chemistry: Reactions, Mechanisms and Structure”, 4th Ed., Wiley-Interscience, New York, 1992; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Biological Assays

The biological activities of the compounds of the present disclosure can be assessed with methods and assays known in the art.

For example, binding to VHL may be determined by fluorescence polarization (FP) assay. In a typical FP assay, serial dilutions of test compounds, fluorescein-labeled tracer, and VBC protein are added to each well. The final volume in the assay buffer (e.g., PBS pH 7.4+0.01% BGG+0.01% Tween-20, 2 mM DTT) is, e.g., 100 μL. The final concentration of fluorescein-labeled tracer and VBC protein is, e.g., 5 nM. The plate is mixed on a shaker for certain period of time (e.g., 15 min) and is incubated at certain temperature (e.g., room temperature) for certain period of time (e.g., 1 h) to reach equilibrium. The polarization values in millipolarization (mP) units are measured at certain excitation wavelength (e.g., 485 nm) and certain emission wavelength (e.g., 530 nm). All experimental data can be analyzed using Prism 8.0 software (GraphPad Software). IC₅₀ values are determined by nonlinear regression fitting of the competition curves. Kᵢ values of competitive inhibitors are obtained directly by nonlinear regression fitting, based upon the KD values of the fluorescein-labeled tracer and concentrations of the protein and probe in the competitive assays.

### Methods of Use

In certain aspects, provided herein are methods of binding VHL E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding VHL E3 ubiquitin ligase protein complex in a subject or biological sample.

In certain aspects, provided herein are compounds described herein for use in binding VHL E3 ubiquitin ligase protein complex in a subject or biological sample.

In certain aspects, provided herein are methods of degrading a protein in a subject or biological sample comprising administering a compound or conjugate described herein to the subject or contacting the biological sample with a compound described herein.

In certain aspects, provided herein are uses of a compound or conjugate described herein in the manufacture of a medicament for degrading a protein in a subject or biological sample.

In certain aspects, provided herein are compounds or conjugates described herein for use in degrading a protein in a subject or biological sample.

In certain aspects, provided herein are methods of reducing a protein in a subject or biological sample comprising administering a compound or conjugate described herein to the subject or contacting the biological sample with a compound described herein.

In certain aspects, provided herein are uses of a compound or conjugate described herein in the manufacture of a medicament for reducing a protein in a subject or biological sample.

In certain aspects, provided herein are compounds or conjugates described herein for use in reducing a protein in a subject or biological sample.

In certain embodiments, the protein is SMARCA2, SMARCA4, STAT3, STAT5, STAT6, KRAS G12C, KRAS G12D, SHP2, AR, ER, EGFR, WDR5, KRAS G12V, or SOS1.

In certain aspects, provided herein are methods of treating or preventing a disease or disorder a subject in need thereof, comprising administering to the subject a compound or conjugate described herein.

In certain aspects, provided herein are uses of a compound or conjugate described herein in the manufacture of a medicament for treating or preventing a disease or disorder in a subject in need thereof.

In certain aspects, provided herein are compounds or conjugates described herein for use in treating or preventing a disease or disorder in a subject in need thereof.

In certain embodiments, the disease or disorder is SMARCA2-, SMARCA4-, STAT3-, STAT5-, STAT6-, KRAS-G12C-, KRAS-G12D-, SHP2-, AR-, ER-, EGFR-, WDR5-, KRAS-G12V-, or SOS1-mediated disease or disorder.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂₋₉ alkenyl”). In certain embodiments, an alkenyl group has 2 to 8 carbon atoms (“C₂ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂-10 alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“C₁₋₁₀ heteroalkyl”). In certain embodiments, a heteroalkyl group is a saturated group having 1 to 9 carbon atoms and 1, 2, 3, or 4 heteroatoms (“C₁₋₉ heteroalkyl”). In certain embodiments, a heteroalkyl group is a saturated group having 1 to 8 carbon atoms and 1, 2, 3, or 4 heteroatoms (“C₁₋₈ heteroalkyl”). In certain embodiments, a heteroalkyl group is a saturated group having 1 to 7 carbon atoms and 1, 2, 3, or 4 heteroatoms (“C₁₋₇ heteroalkyl”). In certain embodiments, a heteroalkyl group is a group having 1 to 6 carbon atoms and 1, 2, or 3 heteroatoms (“C₁₋₆ heteroalkyl”). In certain embodiments, a heteroalkyl group is a saturated group having 1 to 5 carbon atoms and 1 or 2 heteroatoms (“C₁₋₅ heteroalkyl”). In certain embodiments, a heteroalkyl group is a saturated group having 1 to 4 carbon atoms and/or 2 heteroatoms (“C₁₋₄ heteroalkyl”). In certain embodiments, a heteroalkyl group is a saturated group having 1 to 3 carbon atoms and 1 heteroatom (“C₁₋₃ heteroalkyl”). In certain embodiments, a heteroalkyl group is a saturated group having 1 to 2 carbon atoms and 1 heteroatom (“C₁₋₂ heteroalkyl”). In certain embodiments, a heteroalkyl group is a saturated group having 1 carbon atom and 1 heteroatom (“C₁ heteroalkyl”). In certain embodiments, a heteroalkyl group is a saturated group having 2 to 6 carbon atoms and 1 or 2 heteroatoms (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted C₁₋₁₀ heteroalkyl. In certain embodiments, the heteroalkyl group is a substituted C₁₋₁₀ heteroalkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“C₂₋₁₀ heteroalkenyl”). In certain embodiments, a heteroalkenyl group has 2 to 9 carbon atoms at least one double bond, and 1, 2, 3, or 4 heteroatoms (“C₂₋₉ heteroalkenyl”). In certain embodiments, a heteroalkenyl group has 2 to 8 carbon atoms, at least one double bond, and 1, 2, 3, or 4 heteroatoms (“C₂₋₈ heteroalkenyl”). In certain embodiments, a heteroalkenyl group has 2 to 7 carbon atoms, at least one double bond, and 1, 2, 3, or 4 heteroatoms (“C₂₋₇ heteroalkenyl”). In certain embodiments, a heteroalkenyl group has 2 to 6 carbon atoms, at least one double bond, and 1, 2, or 3 heteroatoms (“C₂₋₆ heteroalkenyl”). In certain embodiments, a heteroalkenyl group has 2 to 5 carbon atoms, at least one double bond, and 1 or 2 heteroatoms (“C₂₋₅ heteroalkenyl”). In certain embodiments, a heteroalkenyl group has 2 to 4 carbon atoms, at least one double bond, and 1 or 2 heteroatoms (“C₂₋₄ heteroalkenyl”). In certain embodiments, a heteroalkenyl group has 2 to 3 carbon atoms, at least one double bond, and 1 heteroatom (“C₂₋₃ heteroalkenyl”). In certain embodiments, a heteroalkenyl group has 2 to 6 carbon atoms, at least one double bond, and 1 or 2 heteroatoms (“C₂₋₆ heteroalkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted C₂₋₁₀ heteroalkenyl. In certain embodiments, the heteroalkenyl group is a substituted C₂₋₁₀ heteroalkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“C₂₋₁₀ heteroalkynyl”). In certain embodiments, a heteroalkynyl group has 2 to 9 carbon atoms, at least one triple bond, and 1, 2, 3, or 4 heteroatoms (“C₂₋₉ heteroalkynyl”). In certain embodiments, a heteroalkynyl group has 2 to 8 carbon atoms, at least one triple bond, and 1, 2, 3, or 4 heteroatoms (“C₂₋₈ heteroalkynyl”). In certain embodiments, a heteroalkynyl group has 2 to 7 carbon atoms, at least one triple bond, and 1, 2, 3, or 4 heteroatoms (“C₂₋₇ heteroalkynyl”). In certain embodiments, a heteroalkynyl group has 2 to 6 carbon atoms, at least one triple bond, and 1, 2, or 3 heteroatoms (“C₂₋₆ heteroalkynyl”). In certain embodiments, a heteroalkynyl group has 2 to 5 carbon atoms, at least one triple bond, and 1 or 2 heteroatoms (“C₂₋₅ heteroalkynyl”). In certain embodiments, a heteroalkynyl group has 2 to 4 carbon atoms, at least one triple bond, and lor 2 heteroatoms (“C₂₋₄ heteroalkynyl”). In certain embodiments, a heteroalkynyl group has 2 to 3 carbon atoms, at least one triple bond, and 1 heteroatom (“C₂₋₃ heteroalkynyl”). In certain embodiments, a heteroalkynyl group has 2 to 6 carbon atoms, at least one triple bond, and 1 or 2 heteroatoms (“C₂₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted C₂-10 heteroalkynyl. In certain embodiments, the heteroalkynyl group is a substituted C₂-10 heteroalkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

5-membered heteroaryl containing one heteroatom includes, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl (C₅) and cyclohexyl (C₅). Examples of C₃₋₆ carbocyclyl include the aforementioned C₅₋₆ carbocyclyl groups as well as cyclopropyl (C₃) and cyclobutyl (C₄). Examples of C₃₋₈ carbocyclyl include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

In certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share one common bond with, one or more carbocyclyl groups, as defined above, wherein the point of attachment is on any of the fused rings. In such instances, the number of carbons designates the total number of carbons in the fused carbocyclyl ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on any of the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on any of the carbocyclyl rings in which the spiro structure is embedded.

“Bridged carbocyclyl” or “bridged carbocycle” refers to ring systems wherein the carbocyclyl group, as defined above, form bridged structure with, i.e., share more than one atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the bridged rings. When substitution is indicated, unless otherwise specified, substitution can occur on any of the carbocyclyl rings in which the bridged structure is embedded.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

In certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share one common bond with, one or more heterocyclyl or carbocyclyl groups, as defined above, wherein the point of attachment is on any of the fused rings. In such instances, the number of carbons designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than one atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the bridged rings.

“Hetero” when used to describe a compound or a group present on a compound means that one or more carbon atoms in the compound or group have been replaced by a nitrogen, oxygen, sulfur, boron, phosphorus, or silicon heteroatom, as valency permits. Hetero may be applied to any of the hydrocarbyl groups described above having from 1 to 5, and particularly from 1 to 3 heteroatoms.

“Alkoxy” as used herein, refers to the group —OR, wherein R is alkyl, carbocyclyl, or heterocyclyl as defined herein. C₁₋₆ alkoxy refers to the group —OR, wherein each R is C₁₋₆ alkyl, C₃₋₆ carbocyclyl, or 3- to 6-membered heterocyclyl, as defined herein. Exemplary C₆₋₁₀ alkyl, C₃₋₆ carbocyclyl, or 3- to 6-membered heterocyclyl is set forth above.

“Alkylamino” as used herein, refers to the group —NHR or —NR₂, wherein each R is independently alkyl, carbocyclyl, or heterocyclyl, as defined herein. C₁₋₆ alkylamino refers to the group —NHR or —NR₂, wherein each R is independently C₁₋₆ alkyl, C₃₋₆ carbocyclyl, or 3- to 6-membered heterocyclyl as defined herein. Exemplary C₁₋₆ alkyl, C₃₋₆ carbocyclyl, or 3- to 6-membered heterocyclyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thio-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The disclosure is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g., infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Exemplary Embodiments—I

EXEMPLARY EMBODIMENT B1. A conjugate of Formula II:

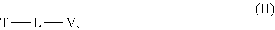

or a pharmaceutically acceptable salt, solvate, or stereoisomer thereof, wherein:
V is of Formula II-1

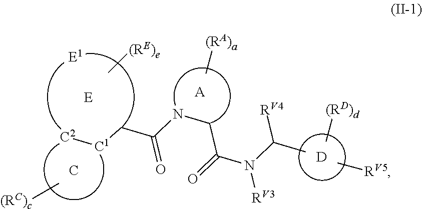

wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

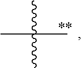

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

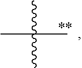

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

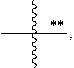

oxo, hydrogen, halogen, —CN, —NO₂, —OH, —NH₂, C₁₋₆ alkyl, CI-6 alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

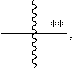

hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is

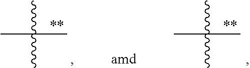

denotes attachment to -L-T,
L is of Formula II-2

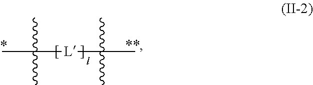

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6,
T is of Formula II-3

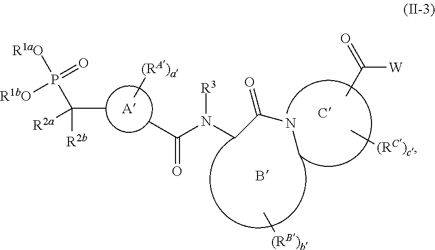

wherein:
R^{1a} and R^{1b} are independently hydrogen or C₁₋₆ alkyl optionally substituted with one or more R^{u};
R^{2a} and R^{2b} are independently hydrogen or halogen;
R^{2a} and R^{2b} together form an oxo; or
R^{2a} and R^{2b}, together with the carbon atom to which they are attached, form C₃₋₆ carbocycle or 3- to 6-membered heterocycle, wherein the carbocycle or heterocycle is optionally substituted with one or more R^{u};
Ring A′ is C₆₋₁₀ aryl or 5- to 10-membered heteroaryl;
each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 6, as valency permits;
R³ is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring B′ is 3- to 12-membered heterocycle;
each R^{B′} is independently

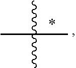

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
b′ is an integer selected from 0 to 6, as valency permits;
Ring C′ is 3- to 8-membered heterocycle;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 6, as valency permits;
W is

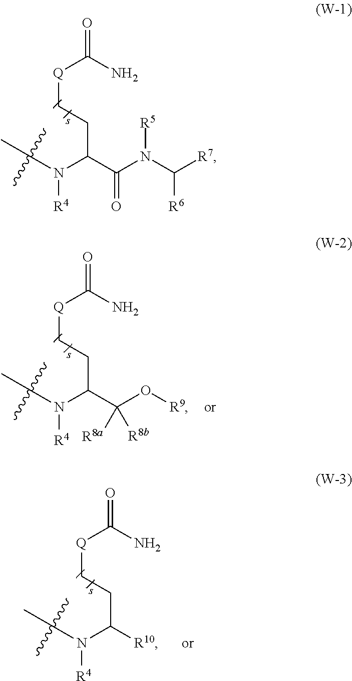

W is

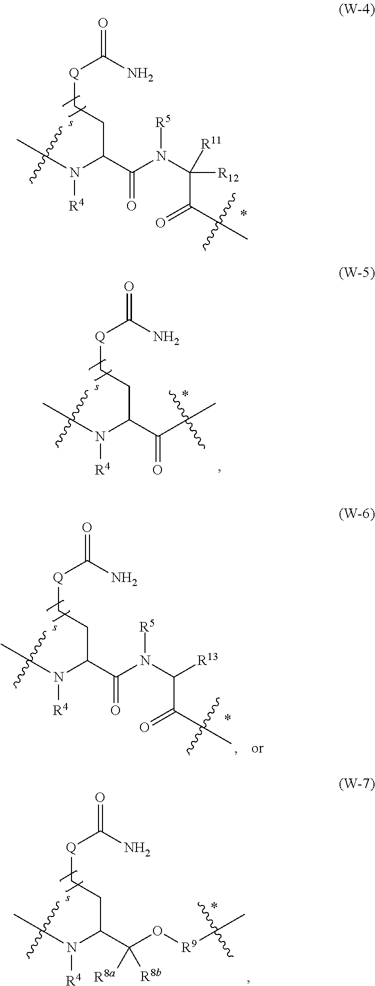

wherein:
* denotes attachment to L;
Q is absent; or
Q is —C(R^{Q})₂—, —O—, and —N(R^{Q′})—;
each R^{Q} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{Q}, together with the carbon atom to which they are attached, form C₃₋₆ carbocyclyl or 3- to 6-membered heterocyclyl, wherein the carbocyclyl or heterocyclyl is optionally substituted with one or more R^{u};
R^{Q′} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u};
s is an integer selected from 0 to 4;
R⁴ and R⁵ are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R⁶ and R⁷ are independently C₆₋₁₀ aryl or 5- to 10-membered heteroaryl, wherein the aryl or heteroaryl is optionally substituted with one or more R^{6a} or R^{7a}, respectively;
each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
R⁷ is hydrogen or C₁₋₆ alkyl optionally substituted with one or more R^{7b};
each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
R⁶ and R⁷, together with the carbon atom to which they are attached, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
R^{8a} and R^{8b} are independently hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, carbocyclyl, or heterocyclyl is optionally substituted with one or more R^{u};
R⁹ is C₆₋₁₀ aryl or 5- to 10-membered heteroaryl, wherein the aryl or heteroaryl is optionally substituted with one or more R^{9a};
each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹⁰ is hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
R¹¹ is hydrogen or C₁₋₆ alkyl optionally substituted with one or more R^{u};
R¹² is hydrogen, C₁₋₆ alkyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a};
each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹¹ and R¹², together with the carbon atom to which they are attached, form C₃₋₆ carbocyclyl or 3- to 6-membered heterocyclyl, wherein carbocyclyl or heterocyclyl is optionally substituted with one or more R^{u}; or
R¹¹ and R⁵, together with the intervening atoms, form 4- to 8-membered heterocyclyl optionally substituted with one or more R^{u}; and
R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

EXEMPLARY EMBODIMENT B2. The conjugate of any one of the preceding exemplary embodiments, wherein Ring C is 5- to 6-membered heteroaryl comprising at least one nitrogen atoms.

EXEMPLARY EMBODIMENT B3. The conjugate of any one of the preceding exemplary embodiments, wherein Ring C is oxazole, isoxazole, or triazole.

EXEMPLARY EMBODIMENT B4. The conjugate of any one of the preceding exemplary embodiments, wherein Ring C is 1,2,3-triazole.

EXEMPLARY EMBODIMENT B5. The conjugate of any one of the preceding exemplary embodiments, wherein V is of Formula I-1-i, II-1-ii, II-1-iii, or II-1-iv

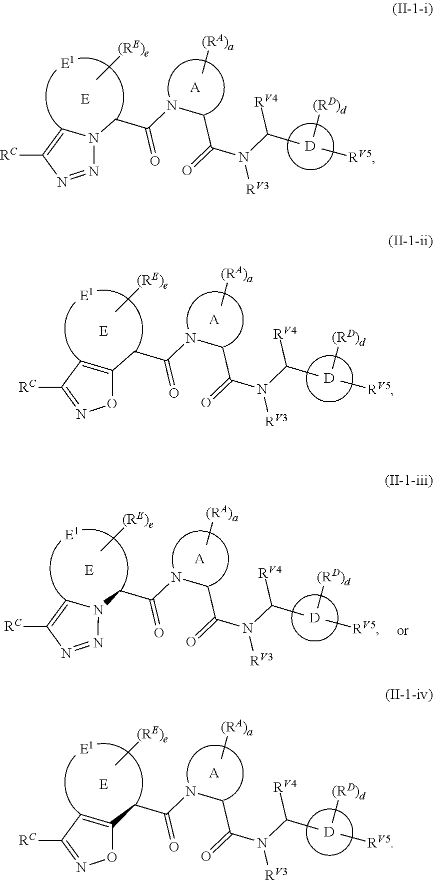

EXEMPLARY EMBODIMENT B6. The conjugate of any one of the preceding exemplary embodiments, wherein Ring E is 5- to 8-membered heterocycle.

EXEMPLARY EMBODIMENT B7. The conjugate of any one of the preceding exemplary embodiments, wherein V is of Formula II-1-v or II-1-vi

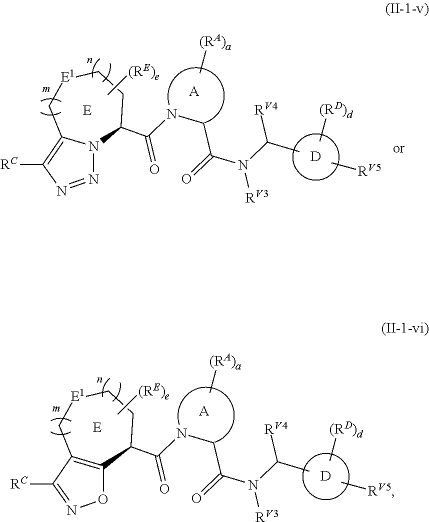

wherein:
m is or 2; and n is 0, 1, or 2.

EXEMPLARY EMBODIMENT 8. The conjugate of any one of the preceding exemplary embodiments, wherein each R^{E} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

EXEMPLARY EMBODIMENT B9. The conjugate of any one of the preceding exemplary embodiments, wherein V is of Formula II-1-vii or II-1-viii

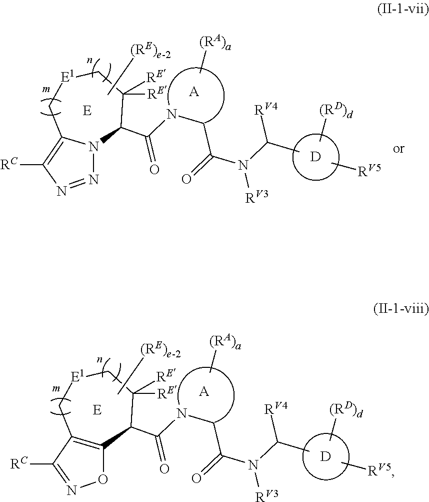

wherein:
R^{E′} is independently hydrogen, C₁₋₆ alkyl, or C₁₋₆ alkoxy, wherein at least one R^{E′} hydrogen;
two R^{E′}, together with the carbon atom to which they are bonded, form C₃₋₆ carbocycle or 3- to 6-membered heterocycle, wherein the carbocycle or heterocycle is optionally substituted with one or more R^{u}; and
e is an integer selected from 2-6.

EXEMPLARY EMBODIMENT 10. The conjugate of any one of the preceding exemplary embodiments, wherein E¹ is C(R^{E1′})₂, NR^{E1′} or O, wherein R^{E1′} is hydrogen or C₁₋₆ alkyl, and R^{E1″} is amino-protecting group, hydrogen, C₁₋₆ alkyl, or —C(═O)R^{a}.

EXEMPLARY EMBODIMENT 11. The conjugate of any one of the preceding exemplary embodiments, wherein each R^{C} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

EXEMPLARY EMBODIMENT B12. The conjugate of any one of the preceding exemplary embodiments, wherein each R^{C} is independently hydrogen, C₁₋₆ alkyl, or C₃₋₆ carbocyclyl.

EXEMPLARY EMBODIMENT B13. The conjugate of any one of the preceding exemplary embodiments, wherein c is 0 or 1.

EXEMPLARY EMBODIMENT B14. The conjugate of any one of the preceding exemplary embodiments, wherein

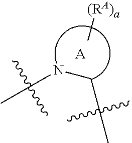

is

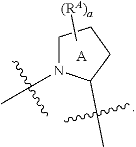

EXEMPLARY EMBODIMENT B15. The conjugate of any one of the preceding exemplary embodiments, wherein a is 1 or 2, and at least one of R^{A} is —OH.

EXEMPLARY EMBODIMENT B16. The conjugate of any one of the preceding exemplary embodiments, wherein

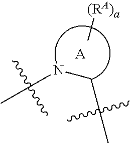

is

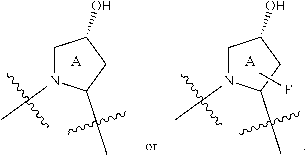

EXEMPLARY EMBODIMENT B17. The conjugate of any one of the preceding exemplary embodiments, wherein Ring D is phenyl.

EXEMPLARY EMBODIMENT B18. The conjugate of any one of the preceding exemplary embodiments, wherein

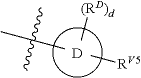

is

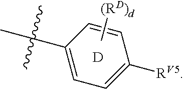

EXEMPLARY EMBODIMENT B19. The conjugate of any one of the preceding exemplary embodiments, wherein d is 0 or 1.

EXEMPLARY EMBODIMENT B20. The conjugate of any one of the preceding exemplary embodiments, wherein R^{V3} is hydrogen.

EXEMPLARY EMBODIMENT B21. The conjugate of any one of the preceding exemplary embodiments, wherein R^{V4} is hydrogen or methyl.

EXEMPLARY EMBODIMENT B22. The conjugate of any one of the preceding exemplary embodiments, wherein R^{V5} is halogen or 5- to 6-membered heteroaryl optionally substituted with one or more R^{u}.

EXEMPLARY EMBODIMENT B23. The conjugate of any one of the preceding exemplary embodiments, wherein R^{V5} is thiazolyl optionally substituted with one or more R^{u}.

EXEMPLARY EMBODIMENT B24. The conjugate of any one of the preceding exemplary embodiments, wherein each L′ is independently C₁₋₆ alkylene, C₁₋₆ heteroalkylene, 5- to 10-membered heteroarylene, C₃₋₁₂ carbocyclylene, 3- to 12-membered heterocyclylene, —C(═O)—, —C(═O)N(R^{L′})—, —C(═O)O—, —N(R^{L′})—, —O—, or —S(═O)₂—, wherein the alkylene, heteroalkylene, heteroarylene, carbocyclylene, or heterocyclylene is optionally substituted with one or more R^{u}, l is an integer selected from 1 to 4.

EXEMPLARY EMBODIMENT B25. The conjugate of any one of the preceding exemplary embodiments, wherein L′ is C₁₋₆ alkylene, C₁₋₆ heteroalkylene, C₃₋₁₂ carbocyclylene, 3- to 12-membered heterocyclylene, —C(═O)—, —N(R^{L′})—, or —O—, wherein the alkylene, carbocyclylene, or heterocyclylene is optionally substituted with one or more R^{u}.

EXEMPLARY EMBODIMENT B26. The conjugate of any one of the preceding exemplary embodiments, wherein L is C₁₋₆ alkylene, —(C₁₋₆ alkylene)-(5- to 10-membered heteroarylene)-(C₁₋₆ alkylene)-, —(C₁₋₆ alkylene)-N(R^{L′})C(═O)—, —C(═O)—(C₁₋₆ alkylene)-N(R^{L′})C(═O)—(C₁₋₆ alkylene)-, or —(C₁₋₆ alkylene)-N(R^{L′})C(═O)—(C₁₋₆ alkylene)-, wherein the alkylene is optionally substituted with one or more R^{u}.

EXEMPLARY EMBODIMENT B27. The conjugate of any one of the preceding exemplary embodiments, wherein each R^{L′} is hydrogen.

EXEMPLARY EMBODIMENT B28. The conjugate of any one of the preceding exemplary embodiments, wherein Ring A′ is 5- to 10-membered heteroaryl.

EXEMPLARY EMBODIMENT B29. The conjugate of any one of the preceding exemplary embodiments, wherein Ring A′ is indolyl or benzo[b]thiophenyl.

EXEMPLARY EMBODIMENT B30. The conjugate of any one of the preceding exemplary embodiments, wherein Ring A′ is

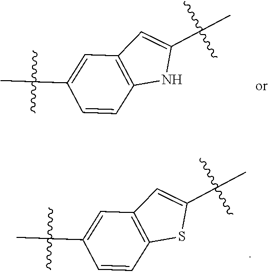 [SMILES: *c1ccc2[nH]c(*)cc2c1 ; *c1ccc2sc(*)cc2c1]

EXEMPLARY EMBODIMENT B31. The conjugate of any one of the preceding exemplary embodiments, wherein T is of Formula II-3-i-a or II-3-i-b

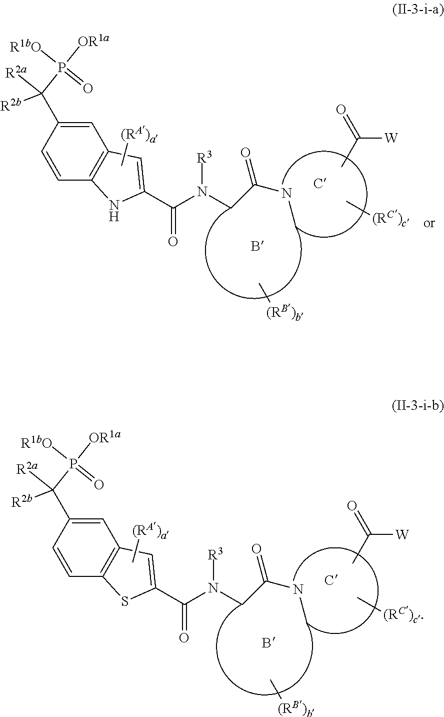

EXEMPLARY EMBODIMENT B32. The conjugate of any one of the preceding exemplary embodiments, wherein Ring B′ is 7- to 9-membered heterocycle.

EXEMPLARY EMBODIMENT B33. The conjugate of any one of the preceding exemplary embodiments, wherein

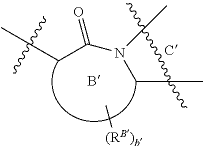

is

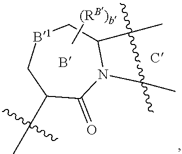

wherein B′¹ is —NHCH₂—, —CH₂NH—, or —CH₂CH₂—.

EXEMPLARY EMBODIMENT B34. The conjugate of any one of the preceding exemplary embodiments, wherein

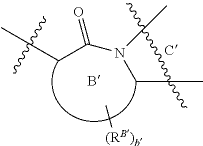

is

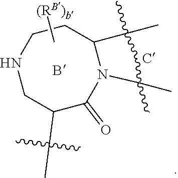

EXEMPLARY EMBODIMENT B35. The conjugate of any one of the preceding exemplary embodiments, wherein Ring C′ is 4- to 7-membered heterocycle.

EXEMPLARY EMBODIMENT B36. The conjugate of any one of the preceding exemplary embodiments, wherein T is of Formula II-3-ii-a or II-3-ii-b

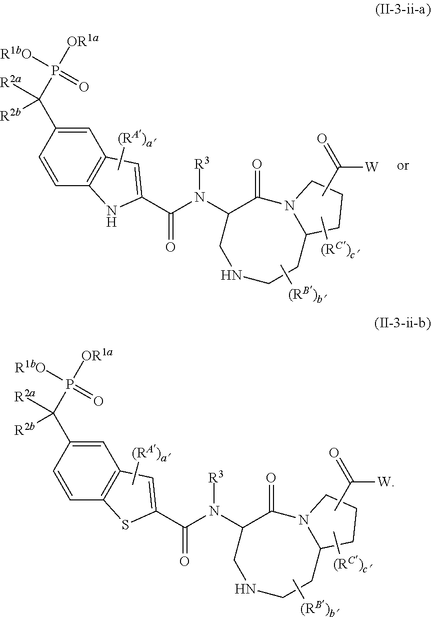

EXEMPLARY EMBODIMENT B37. The conjugate of any one of the preceding exemplary embodiments, wherein R^{1a} and R^{1b} are both hydrogen.

EXEMPLARY EMBODIMENT B38. The conjugate of any one of the preceding exemplary embodiments, wherein R^{2a} and R^{2b} are independently halogen.

EXEMPLARY EMBODIMENT B39. The conjugate of any one of the preceding exemplary embodiments, wherein R^{2a} and R^{2b} are both —F.

EXEMPLARY EMBODIMENT B40. The conjugate of any one of the preceding exemplary embodiments, wherein R^{2a} and R^{2b} together form an oxo.

EXEMPLARY EMBODIMENT B41. The conjugate of any one of the preceding exemplary embodiments, wherein each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B42. The conjugate of any one of the preceding exemplary embodiments, wherein a′ is 0.

EXEMPLARY EMBODIMENT B43. The conjugate of any one of the preceding exemplary embodiments, wherein R³ is hydrogen or C₁₋₆ alkyl.

EXEMPLARY EMBODIMENT B44. The conjugate of any one of the preceding exemplary embodiments, wherein each R^{B′} is independently

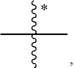

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B45. The conjugate of any one of the preceding exemplary embodiments, wherein each R^{B′} is independently

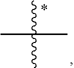

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B46. The conjugate of any one of the preceding exemplary embodiments, wherein b′ is 0 or 1.

EXEMPLARY EMBODIMENT B47. The conjugate of any one of the preceding exemplary embodiments, wherein each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B48. The conjugate of any one of the preceding exemplary embodiments, wherein c′ is 0.

EXEMPLARY EMBODIMENT B49. The conjugate of any one of the preceding exemplary embodiments, wherein W is

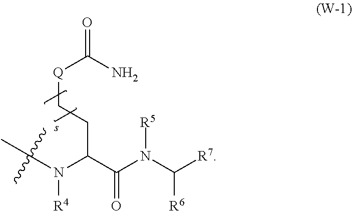

EXEMPLARY EMBODIMENT B50. The conjugate of any one of the preceding exemplary embodiments, wherein R⁶ and R⁷ are independently C₆ aryl or 5- to 6-membered heteroaryl, wherein the aryl or heteroaryl is optionally substituted with one or more R^{6a} or R^{7b}, respectively.

EXEMPLARY EMBODIMENT B51. The conjugate of any one of the preceding exemplary embodiments, wherein each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —S(═O)₂R^{a}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B52. The conjugate of any one of the preceding exemplary embodiments, wherein each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B53. The conjugate of any one of the preceding exemplary embodiments, wherein W is

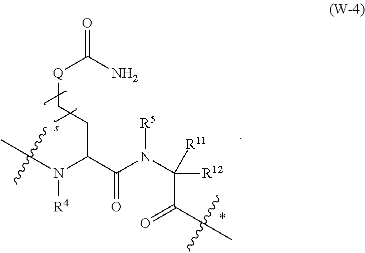

EXEMPLARY EMBODIMENT B54. The conjugate of any one of the preceding exemplary embodiments, wherein R¹² is —(C₁₋₆ alkylene)-(C₆₋₁₀ aryl) or —(C₁₋₆ alkylene)-(5- to 10-membered heteroaryl), wherein the alkylene is optionally substituted with one or more R^{u}, and the aryl or heteroaryl is optionally substituted with one or more R^{12a}.

EXEMPLARY EMBODIMENT B55. The conjugate of any one of the preceding exemplary embodiments, wherein each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —S(═O)₂R^{a}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B56. The conjugate of any one of the preceding exemplary embodiments, wherein each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —N^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B57. The conjugate of any one of the preceding exemplary embodiments, wherein R⁵ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

EXEMPLARY EMBODIMENT B58. The conjugate of any one of the preceding exemplary embodiments, wherein R⁵ is hydrogen.

EXEMPLARY EMBODIMENT B59. The conjugate of any one of the preceding exemplary embodiments, wherein W is

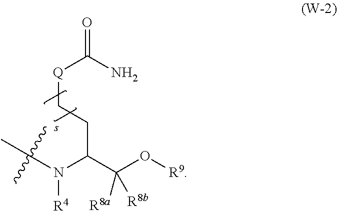

EXEMPLARY EMBODIMENT B60. The conjugate of any one of the preceding exemplary embodiments, wherein W is

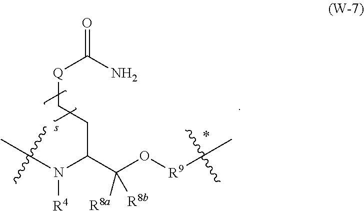

EXEMPLARY EMBODIMENT B61. The conjugate of any one of the preceding exemplary embodiments, wherein R^{8a} and R^{8b} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

EXEMPLARY EMBODIMENT B62. The conjugate of any one of the preceding exemplary embodiments, wherein R^{8a} and R^{8b} are both hydrogen.

EXEMPLARY EMBODIMENT B63. The conjugate of any one of the preceding exemplary embodiments, wherein R⁹ is C₆ aryl or 5- to 6-membered heteroaryl, wherein the aryl or heteroaryl is optionally substituted with one or more R^{9a}.

EXEMPLARY EMBODIMENT B64. The conjugate of any one of the preceding exemplary embodiments, wherein each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, or —S(═O)₂R^{a}, wherein the alkyl, alkoxy, alkylamino, alkenyl, alkynyl, carbocyclyl, heterocyclyl, aryl, or heteroaryl is optionally substituted with one or more R^{u};

EXEMPLARY EMBODIMENT B65. The conjugate of claim 63, wherein each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

EXEMPLARY EMBODIMENT B66. The conjugate of any one of the preceding exemplary embodiments, wherein R⁴ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

EXEMPLARY EMBODIMENT B67. The conjugate of any one of the preceding exemplary embodiments, wherein R⁴ is hydrogen.

EXEMPLARY EMBODIMENT B68. The conjugate of any one of the preceding exemplary embodiments, wherein Q is absent.

EXEMPLARY EMBODIMENT B69. The conjugate of any one of the preceding exemplary embodiments, wherein s is 1.

EXEMPLARY EMBODIMENT B70. A conjugate selected from compounds described in Table 2 and pharmaceutically acceptable salts thereof.

EXEMPLARY EMBODIMENT B71. A pharmaceutical composition comprising the conjugate of any one of the preceding exemplary embodiments, and a pharmaceutically acceptable excipient.

EXEMPLARY EMBODIMENT B72. A method of degrading STAT3 protein in a patient or biological sample comprising contacting said patient or biological sample with a conjugate of any one of the preceding exemplary embodiments.

EXEMPLARY EMBODIMENT B73. Use of a conjugate of any one of the preceding exemplary embodiments in the manufacture of a medicament for degrading STAT3 protein in a patient or biological sample.

EXEMPLARY EMBODIMENT B74. A conjugate of any one of the preceding exemplary embodiments for use in degrading STAT3 protein in a patient or biological sample.

EXEMPLARY EMBODIMENT B75. A method of treating a STAT3-mediated disease or disorder comprising administering to a patient in need thereof a conjugate of any one of the preceding exemplary embodiments.

EXEMPLARY EMBODIMENT B76. Use of a conjugate of any one of the preceding exemplary embodiments in the manufacture of a medicament for treating a STAT3-mediated disease or disorder.

EXEMPLARY EMBODIMENT B77. A conjugate of any one of the preceding exemplary embodiments for use in treating a STAT3-mediated disease or disorder.

EXEMPLARY EMBODIMENT B78. The method, use, or conjugate for use of any one of the preceding exemplary embodiments, wherein the STAT3-mediated disorder is cancer, an autoimmune disease, an inflammatory disorder, a neurodegenerative disease, a viral disease, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, or a CNS disorder.

### Exemplary Embodiments—II

EXEMPLARY EMBODIMENT A1. A compound of Formula I:

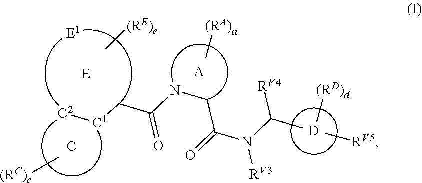

or a pharmaceutically acceptable salt, solvate, or stereoisomer thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —N₃, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₁₂ carbocyclyl, 3- to 12-membered heterocyclyl, C₆₋₁₀ aryl, or 5- to 10-membered heteroaryl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

EXEMPLARY EMBODIMENT A2. A conjugate of Formula I:

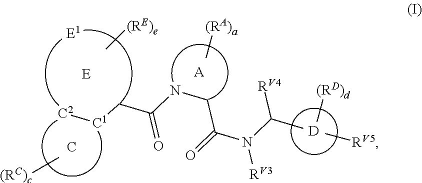

or a pharmaceutically acceptable salt, solvate, or stereoisomer thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

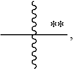

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

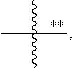

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

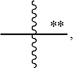

oxo, hydrogen, halogen, —CN, —NO₂, —OH, —NH₂, C₁₋₆ alkyl, CI-6 alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

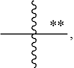

hydrogen, C₆₋₁₀ alkyl, C₁₋₈ heteroalkyl, wherein the alkyl, C₁₋₆ alkylene, or heteroalkyl is optionally substituted with one or more R^{u};
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{V4}, R^{E}, R^{D}, R^{C} is

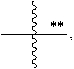

wherein

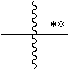

denotes attachment to -L-T,
L is a linker; and
T is a ligand for a protein,
wherein:
each R^{u} is independently oxo, halogen, —N₃, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

EXEMPLARY EMBODIMENT A3. The compound or conjugate of any one of the preceding exemplary embodiments, wherein Ring C is 5- to 6-membered heteroaryl comprising at least one nitrogen atom.

EXEMPLARY EMBODIMENT A4. The compound or conjugate of any one of the preceding exemplary embodiments, wherein Ring C is oxazole, isoxazole, or triazole.

EXEMPLARY EMBODIMENT A5. The compound or conjugate of any one of the preceding exemplary embodiments, wherein Ring C is 1,2,3-triazole.

EXEMPLARY EMBODIMENT A6. The compound or conjugate of any one of the preceding exemplary embodiments, wherein the compound is a compound of Formula I-0, I-0′, I-1, or I-2

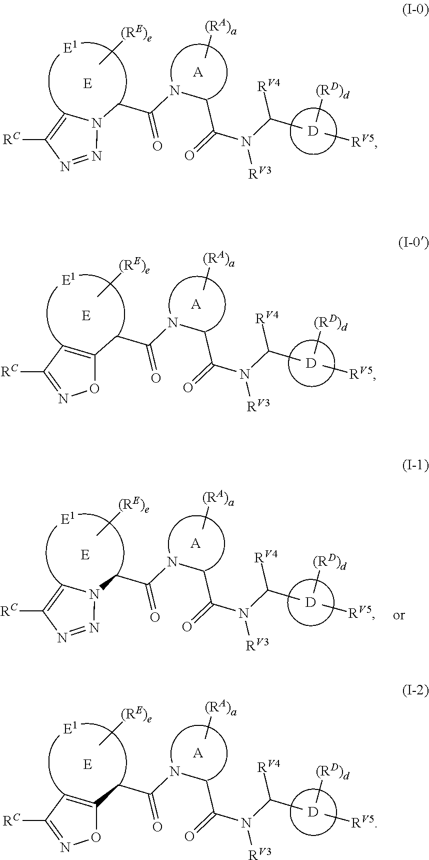

EXEMPLARY EMBODIMENT A7. The compound or conjugate of any one of the preceding exemplary embodiments, wherein Ring E is 5- to 8-membered heterocycle or C₅₋₈ carbocycle.

EXEMPLARY EMBODIMENT A8. The compound or conjugate of any one of the preceding exemplary embodiments, wherein the compound is a compound of Formula I-1-i or I-2-i

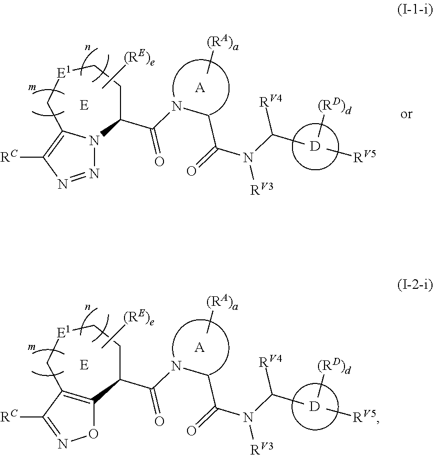

wherein:
m is 1 or 2; and n is 0, 1, or 2.

EXEMPLARY EMBODIMENT A9. The compound or conjugate of any one of the preceding exemplary embodiments, wherein each R^{E} is independently oxo, hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

EXEMPLARY EMBODIMENT A10. The compound or conjugate of any one of the preceding exemplary embodiments, wherein the compound is a compound of Formula I-1-ii or I-2-ii

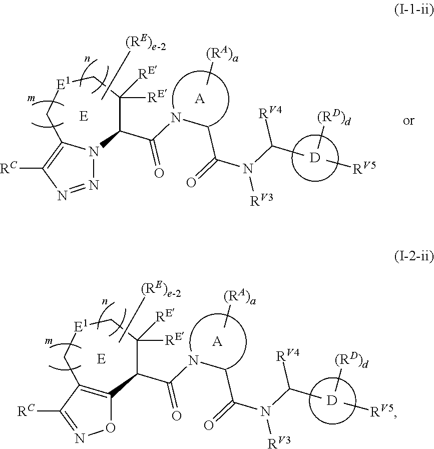

wherein:
R^{E′} is independently hydrogen, C₁₋₆ alkyl, or C₁₋₆ alkoxy, wherein at least one R^{E′} i o hydrogen, or
two R^{E′}, together with the carbon atom to which they are bonded, form C₃₋₆ carbocycle or 3- to 6-membered heterocycle, wherein the carbocycle or heterocycle is optionally substituted with one or more R^{u}; and
e is an integer selected from 2 to 6.

EXEMPLARY EMBODIMENT A11. The compound or conjugate of any one of the preceding exemplary embodiments, wherein E¹ is C(R^{E1′})₂, NR^{E1″}, or O, wherein R^{E1′} is hydrogen or C₁₋₆ alkyl, and R^{E1″} is amino-protecting group, hydrogen, C₁₋₆ alkyl, or —C(═O)R^{a}.

EXEMPLARY EMBODIMENT A12. The compound or conjugate of any one of the preceding exemplary embodiments, wherein

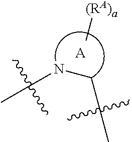

is

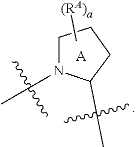

EXEMPLARY EMBODIMENT A16. The compound or conjugate of any one of the preceding exemplary embodiments, wherein a is 1 or 2, and at least one of R^{A} is —OH.

EXEMPLARY EMBODIMENT A17. The compound or conjugate of any one of the preceding exemplary embodiments, wherein

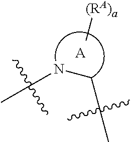

is

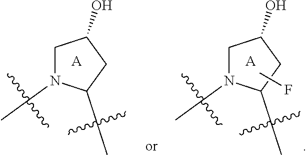

EXEMPLARY EMBODIMENT A18. The compound or conjugate of any one of the preceding exemplary embodiments, wherein Ring D is phenyl.

EXEMPLARY EMBODIMENT A19. The compound or conjugate of any one of the preceding exemplary embodiments, wherein

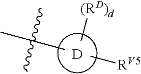

is

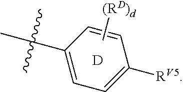

EXEMPLARY EMBODIMENT A20. The compound or conjugate of any one of the preceding exemplary embodiments, wherein d is 0 or 1.

EXEMPLARY EMBODIMENT A21. The compound or conjugate of any one of the preceding exemplary embodiments, wherein R^{V3} is hydrogen.

EXEMPLARY EMBODIMENT A22. The compound or conjugate of any one of the preceding exemplary embodiments, wherein R^{V4} is hydrogen or C₁₋₆ alkyl optionally substituted with one or more R^{u}.

EXEMPLARY EMBODIMENT A23. The compound or conjugate of any one of the preceding exemplary embodiments, wherein R^{V4} is hydrogen, —CH₂C(═O)R^{a}, —CH₂C(═O)OR^{b}, or —CH₂N₃.

EXEMPLARY EMBODIMENT A24. The compound or conjugate of any one of the preceding exemplary embodiments, wherein R^{V5} is halogen or 5- to 6-membered heteroaryl optionally substituted with one or more R^{u}.

EXEMPLARY EMBODIMENT A25. The compound or conjugate of any one of the preceding exemplary embodiments, wherein R^{V5} is thiazolyl optionally substituted with methyl.

EXEMPLARY EMBODIMENT A26. A compound selected from compounds described in Table 1 and pharmaceutically acceptable salts thereof.

EXEMPLARY EMBODIMENT A27. A pharmaceutical composition comprising the compound of any one of the preceding exemplary embodiments, and a pharmaceutically acceptable excipient.

EXEMPLARY EMBODIMENT A28. A method of binding VHL E3 ubiquitin ligase protein complex in a subject or biological sample comprising administering the compound of any one of the preceding exemplary embodiments to the subject or contacting the biological sample with the compound of any one of the preceding exemplary embodiments.

EXEMPLARY EMBODIMENT A29. Use of the compound of any one of the preceding exemplary embodiments in the manufacture of a medicament for binding VHL E3 ubiquitin ligase protein complex in a subject or biological sample.

EXEMPLARY EMBODIMENT A30. A compound of any one of the preceding exemplary embodiments for use in binding VHL E3 ubiquitin ligase protein complex in a subject or biological sample.

EXEMPLARY EMBODIMENT A31. A method of treating or preventing a disease or disorder a subject in need thereof, comprising administering to the subject a conjugate of any one of the preceding exemplary embodiments.

EXEMPLARY EMBODIMENT A32. Use of a conjugate of any one of the preceding exemplary embodiments in the manufacture of a medicament for treating or preventing a disease or disorder in a subject in need thereof.

EXEMPLARY EMBODIMENT A33. A conjugate of any one of the preceding exemplary embodiments for use in treating or preventing a disease or disorder in a subject in need thereof.

EXEMPLARY EMBODIMENT A34. The method, use, or compound for use of any one of the preceding exemplary embodiments, wherein the disease or disorder is an estrogen receptor-mediated disease or disorder, a STAT3-mediated disease or disorder, a SMARCA2/4-mediated disease or disorder, a CBP/p300-mediated disease or disorder, an androgen receptor-mediated disease or disorder, or a BRD9-mediated disease or disorder.

### Exemplary Embodiments—III

EXEMPLARY EMBODIMENT C1. A conjugate of Formula II:

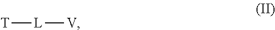

or a pharmaceutically acceptable salt, solvate, or stereoisomer thereof, wherein:
V is of Formula II-1

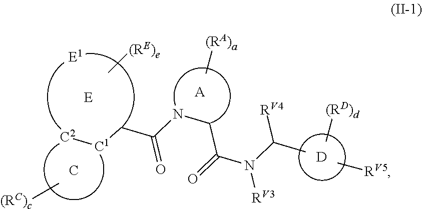

wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently

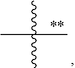

hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently

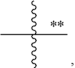

halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1′}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently

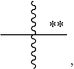

oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is

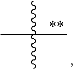

hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is

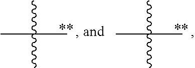

denotes attachment to -L-T,
L is of Formula II-2

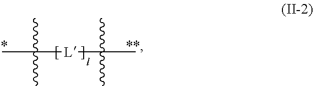

wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6,
T is of Formula II-3

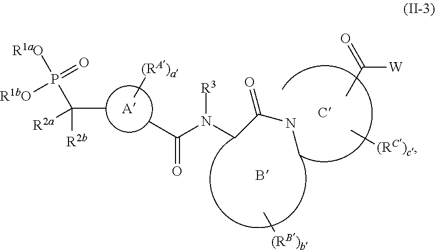

wherein:
R^{1a} and R^{1b} are independently hydrogen or C₁₋₆ alkyl optionally substituted with one or more R^{u};
R^{2a} and R^{2b} are independently hydrogen or halogen;
R^{2a} and R^{2b} together form an oxo; or
R^{2a} and R^{2b}, together with the carbon atom to which they are attached, form C₃₋₆ carbocycle or 3- to 6-membered heterocycle, wherein the carbocycle or heterocycle is optionally substituted with one or more R^{u};
Ring A′ is C₆₋₁₀ aryl or 5- to 10-membered heteroaryl;
each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 6, as valency permits;
R³ is hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring B′ is 3- to 12-membered heterocycle;
each R^{B′} is independently

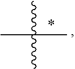

oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
b′ is an integer selected from 0 to 6, as valency permits;
Ring C′ is 3- to 8-membered heterocycle;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 6, as valency permits;
W is

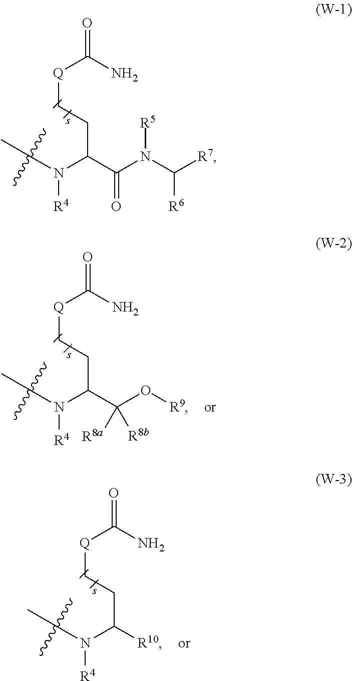

W is

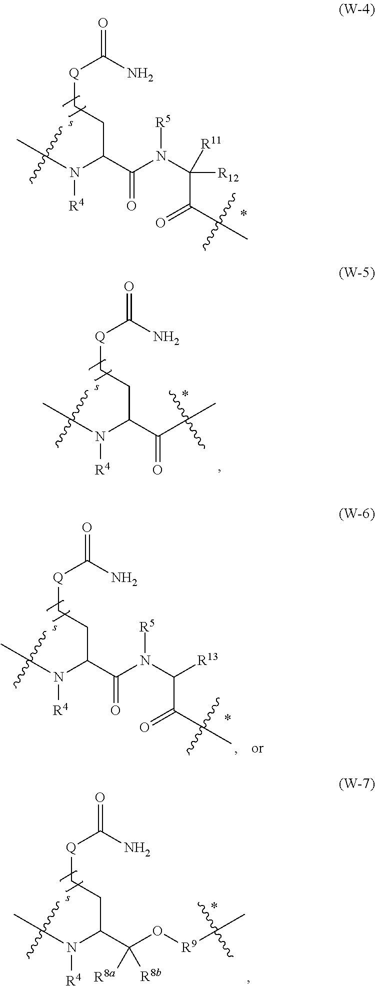

wherein:
* denotes attachment to L;
Q is absent; or
Q is —C(R^{Q})₂—, —O—, and —N(R^{Q′})—;
each R^{Q} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{Q}, together with the carbon atom to which they are attached, form C₃₋₆ carbocyclyl or 3- to 6-membered heterocyclyl, wherein the carbocyclyl or heterocyclyl is optionally substituted with one or more R^{u};
R^{Q′} is hydrogen, C₆₋₁₀ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u};
s is an integer selected from 0 to 4;
R⁴ and R⁵ are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R⁶ and R⁷ are independently C₆₋₁₀ aryl or 5- to 10-membered heteroaryl, wherein the aryl or heteroaryl is optionally substituted with one or more R^{6a} or R^{7a}, respectively;
each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
R⁷ is hydrogen or C₁₋₆ alkyl optionally substituted with one or more R^{7b};
each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
R⁶ and R⁷, together with the carbon atom to which they are attached, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
R^{8a} and R^{8b} are independently hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, carbocyclyl, or heterocyclyl is optionally substituted with one or more R^{u};
R⁹ is C₆₋₁₀ aryl or 5- to 10-membered heteroaryl, wherein the aryl or heteroaryl is optionally substituted with one or more R^{9a};
each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹⁰ is hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
R¹¹ is hydrogen or C₁₋₆ alkyl optionally substituted with one or more R^{u};
R¹² is hydrogen, C₁₋₆ alkyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a};
each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹¹ and R¹², together with the carbon atom to which they are attached, form C₃₋₆ carbocyclyl or 3- to 6-membered heterocyclyl, wherein carbocyclyl or heterocyclyl is optionally substituted with one or more R^{u}; or
R¹¹ and R⁵, together with the intervening atoms, form 4- to 8-membered heterocyclyl optionally substituted with one or more R^{u}; and
R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl, wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

EXEMPLARY EMBODIMENT C2. The compound of EXEMPLARY EMBODIMENT C1, or a pharmaceutically acceptable salt thereof, wherein:
V is of Formula II-1-ix, II-1-x, II-1-xi, II-1-xii, or II-1-xiii:

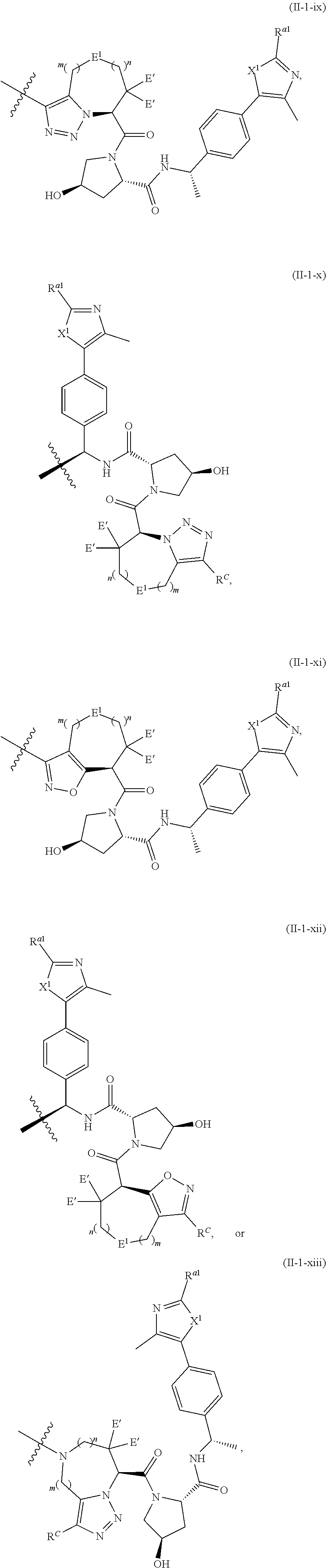

wherein:
R^{a1} is C₁₋₆ alkyl;
X¹ is 0 or S;
E¹ is CH₂, NR^{E1″} or O;
R^{E1″} is hydrogen, C₁₋₆ alkyl, or —C(═O)R^{a};
E′ is hydrogen or C₁₋₆ alkyl;
R^{a} is C₆₋₁₀ alkyl;
R^{C} is C₆₋₁₀ alkyl or C₃₋₆ carbocyclyl;
m is 1 or 2;
n is 0 or 1;
and

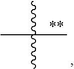

denotes attachment to -L-T;
L is of Formula II-2:

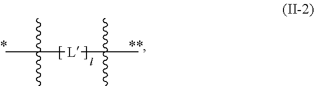

wherein:
* denotes attachment to T and ** denotes attachment to V;
each L′ is independently C₁₋₆ alkylene, 5- to 10-membered heteroarylene, —C(═O)—, —C(═O)N(R^{L′})—, —C(═O)O—, or —N(R^{L′})—;
R^{L′} is hydrogen;
l is an integer selected from 1 to 4;
T is of Formula II-3-ii-g or II-3-ii-h:

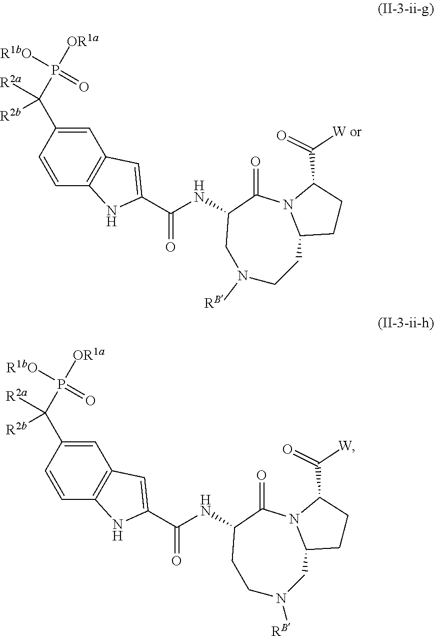

wherein:
R^{1a} and R^{1b} are independently hydrogen or C₁₋₆ alkyl;
R^{2a} and R^{2b} are independently hydrogen or halogen;
R^{2a} and R^{2b} together form an oxo; or
R^{B′} is —C(═O)R^{a};
W is W-7 or W-8:

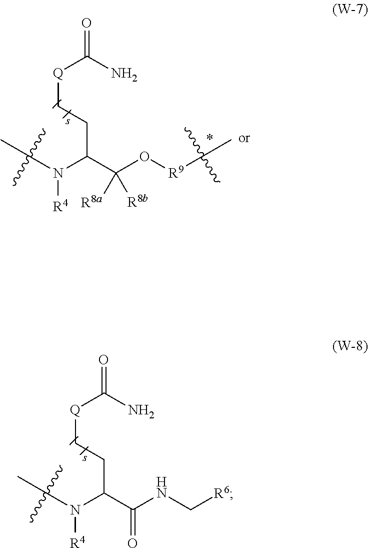

wherein:
* denotes attachment to L;
Q is absent;
s is 1;
R⁴ is hydrogen;
R⁶ is C₆₋₁₀ aryl, wherein the aryl is optionally substituted with one or more R^{6a};
each R^{6a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d};
R^{a} is C₁₋₆ alkyl, C₃₋₁₂ carbocyclyl, 3- to 12-membered heterocyclyl, C₆₋₁₀ aryl, or 5- to 10-membered heteroaryl;
R^{b} is hydrogen, C₁₋₆ alkyl, C₃₋₁₂ carbocyclyl, 3- to 12-membered heterocyclyl, C₆₋₁₀ aryl, or 5- to 10-membered heteroaryl;
R^{c} and R^{d} are independently hydrogen, C₁₋₆ alkyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl;
R^{8a} and R^{8b} are both hydrogen;
R⁹ is C₆₋₁₀ aryl or 5- to 10-membered heteroaryl, wherein the aryl or heteroaryl is optionally substituted with one or more R^{9a}; and
each R^{9a} is independently halogen, —CN, —NO₂, —OH, —NH₂, C₁₋₆ alkyl, C₁₋₆ alkoxy, C₃₋₁₂ carbocyclyl, 3- to 12-membered heterocyclyl, C₆₋₁₀ aryl, or 5- to 10-membered heteroaryl,
with the provisos that:
(i) when R^{B′} is —C(═O)R^{a}, then W is W-7; or
(ii) when R^{B′} is

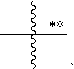

then W is W-8.

EXEMPLARY EMBODIMENT C3. The compound of EXEMPLARY EMBODIMENTS C₁ or C₂, or a pharmaceutically acceptable salt thereof, wherein V is of Formula II-1-xiv or II-1-xv:

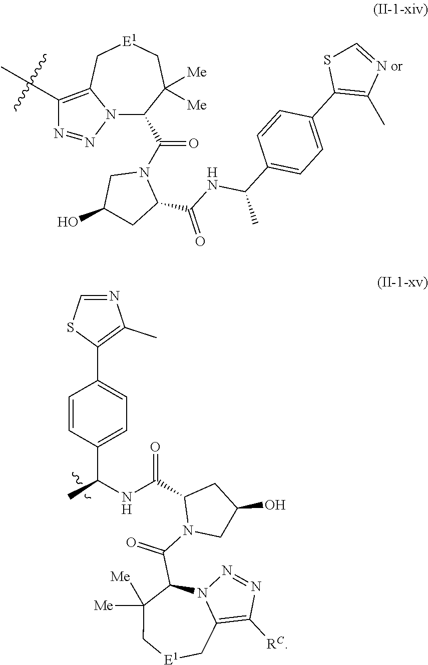

EXEMPLARY EMBODIMENT C4. The compound of any one of EXEMPLARY EMBODIMENTS C₁-C₃, or a pharmaceutically acceptable salt thereof, wherein E¹ is O.

EXEMPLARY EMBODIMENT C5. The compound of any one of EXEMPLARY EMBODIMENTS C₁-C₄, or a pharmaceutically acceptable salt thereof, wherein R^{C} is methyl or cyclopropyl.

EXEMPLARY EMBODIMENT C6. The compound any one of EXEMPLARY EMBODIMENTS C₁-C₅, or a pharmaceutically acceptable salt thereof, wherein each L′ is independently C₁₋₆ alkylene, —C(═O)—, or —N(R^{L′})—.

EXEMPLARY EMBODIMENT C7. The compound of any one of EXEMPLARY EMBODIMENTS C₁-C₆, or a pharmaceutically acceptable salt thereof, wherein T is of Formula II-3-ii-g.

EXEMPLARY EMBODIMENT C8. The compound of EXEMPLARY EMBODIMENT C7, or a pharmaceutically acceptable salt thereof, wherein R^{B′} is —C(═O)CH₃.

EXEMPLARY EMBODIMENT C9. The compound of any one of EXEMPLARY EMBODIMENTS C₁-C₆, or a pharmaceutically acceptable salt thereof, wherein T is of Formula II-3-ii-h.

EXEMPLARY EMBODIMENT C10. The compound of EXEMPLARY EMBODIMENT C9, or a pharmaceutically acceptable salt thereof, wherein R^{B′} is —C(═O)CH₃.

EXEMPLARY EMBODIMENT C11 The conjugate of any one of EXEMPLARY EMBODIMENTS C₁-C₁₀, wherein R⁹ is:

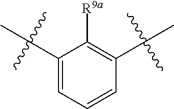

EXEMPLARY EMBODIMENT C12. The conjugate of EXEMPLARY EMBODIMENT C11, or a pharmaceutically acceptable salt thereof, wherein R^{9a} is halogen.

EXEMPLARY EMBODIMENT C13. The conjugate of EXEMPLARY EMBODIMENT C2, or a pharmaceutically acceptable salt thereof, wherein the conjugate is of Formula II-XI:

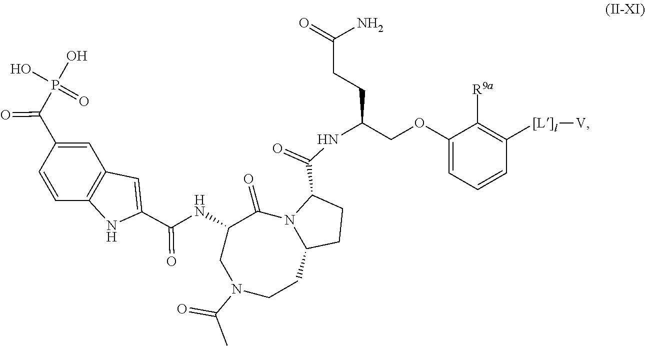

wherein:
R^{9a} is chloro;
each L′ is independently C₁₋₆ alkylene, —C(═O)—, or —N(R^{L′})—;
R^{L′} is hydrogen; and
l is an integer from 1 to 4.

EXEMPLARY EMBODIMENT C14. The conjugate of EXEMPLARY EMBODIMENT C13, or a pharmaceutically acceptable salt thereof, wherein V is:

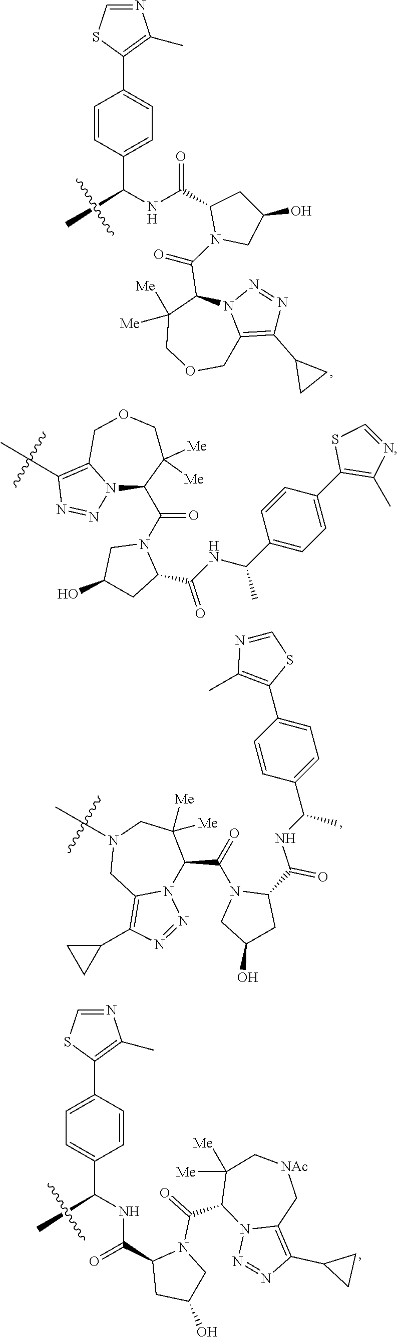

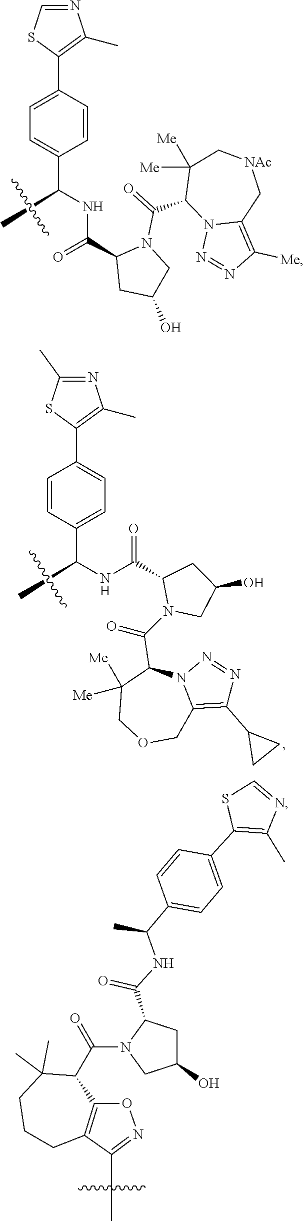

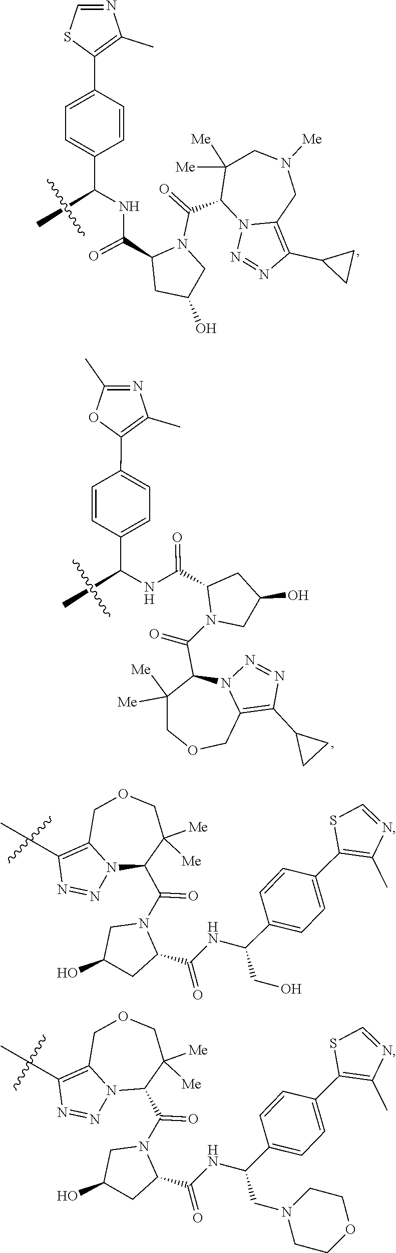

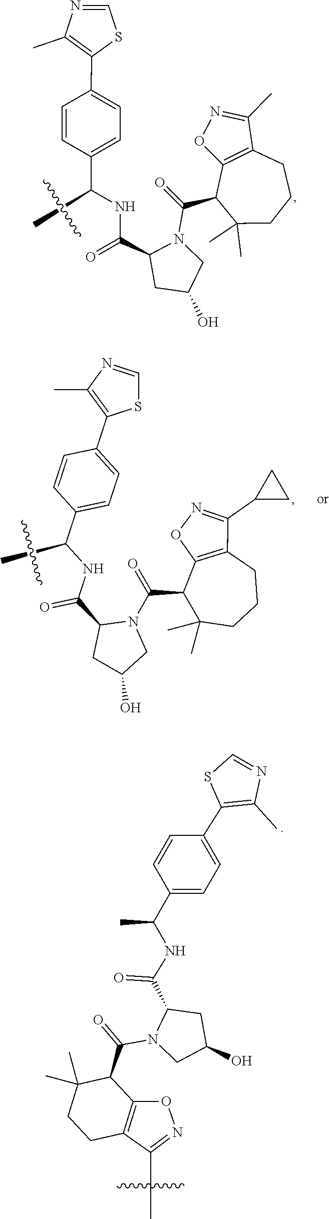

EXEMPLARY EMBODIMENT C15. The conjugate of EXEMPLARY EMBODIMENT C2, or a pharmaceutically acceptable salt thereof, wherein the conjugate is of Formula II-XII:

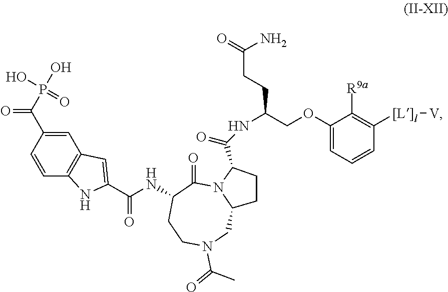

wherein:
R^{9a} is chloro;
each L′ is independently C₁₋₆ alkylene, —C(═O)—, or —N(R^{L′})—;
R^{L′} is hydrogen; and
l is an integer from 1 to 4.

EXEMPLARY EMBODIMENT C16. The conjugate of EXEMPLARY EMBODIMENT C15, or a pharmaceutically acceptable salt thereof, wherein V is:

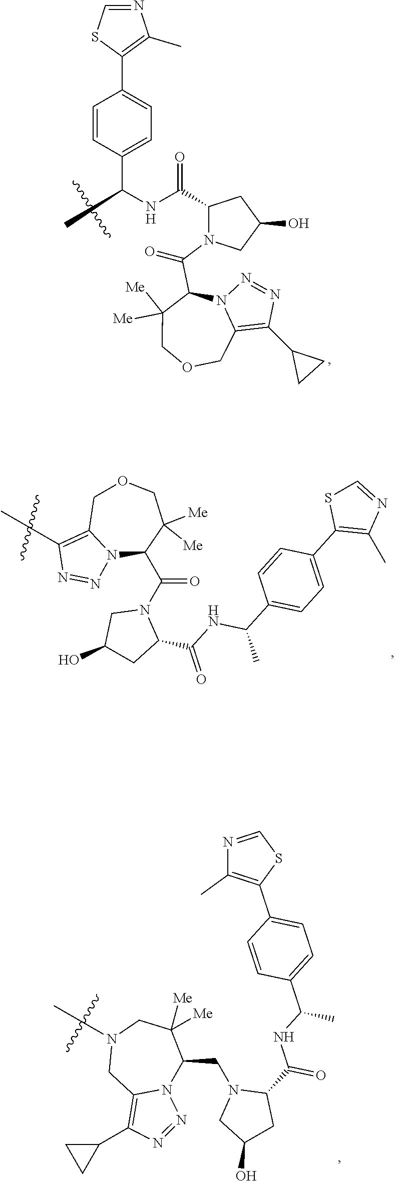 [SMILES: Cc1ncsc1-c1ccc([C@H](C)NC(=O)[C@@H]2C[C@@H](O)CN2C(=O)[C@H]2n3nnc(C4CC4)c3COCC2(C)C)cc1 ; *c1nnn2c1COCC(C)(C)[C@H]2C(=O)N1C[C@H](O)C[C@H]1C(=O)N[C@@H](C)c1ccc(-c2scnc2C)cc1 ; *N1Cc2c(C3CC3)nnn2[C@H](CN2C[C@H](O)C[C@H]2C(=O)N[C@@H](C)c2ccc(-c3scnc3C)cc2)C(C)(C)C1]

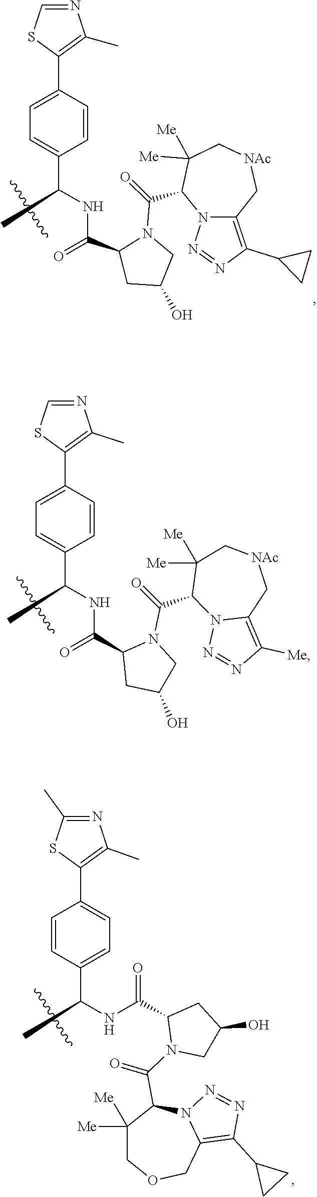

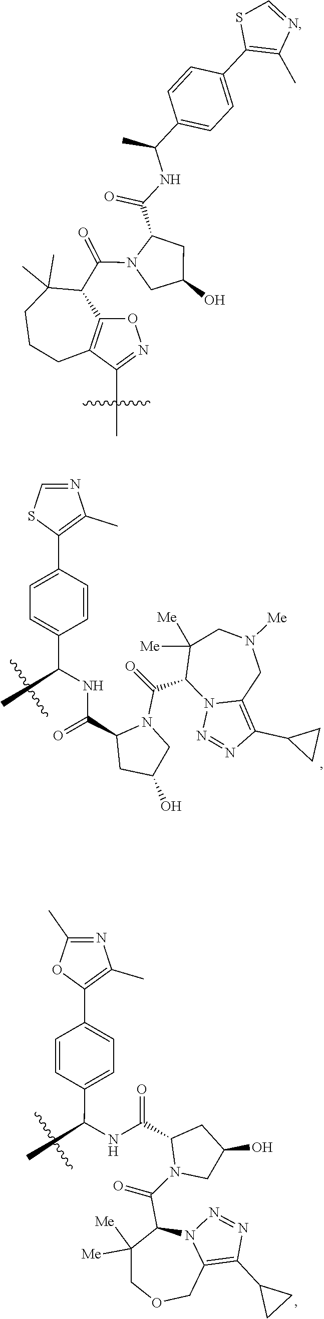

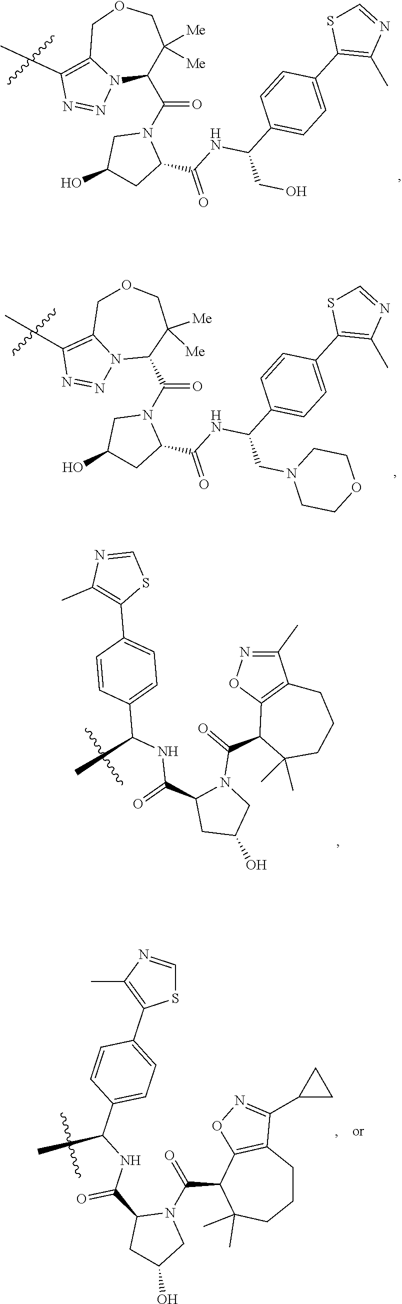

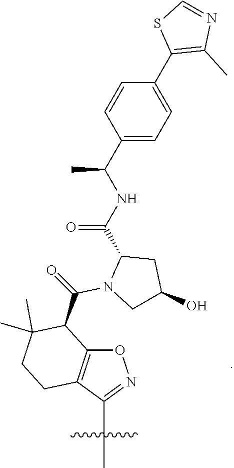

EXEMPLARY EMBODIMENT C17. The conjugate of EXEMPLARY EMBODIMENT C2, or a pharmaceutically acceptable salt thereof, wherein the conjugate is of Formula II-XV:

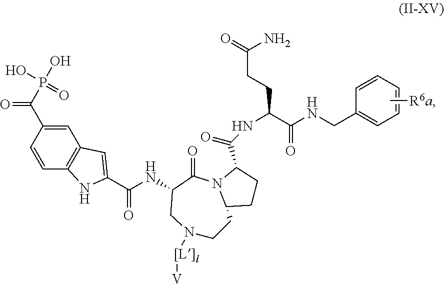

wherein:
R^{6a} is —S(═O)₂R^{a};
R^{a} is C₁₋₆ alkyl;
each L′ is independently C₁₋₆ alkylene, —C(═O)—, or —N(R^{L′})—;
R^{L′} is hydrogen; and
l is an integer from 1 to 4.

EXEMPLARY EMBODIMENT C18. The conjugate of EXEMPLARY EMBODIMENT C17, or a pharmaceutically acceptable salt thereof, wherein V is:

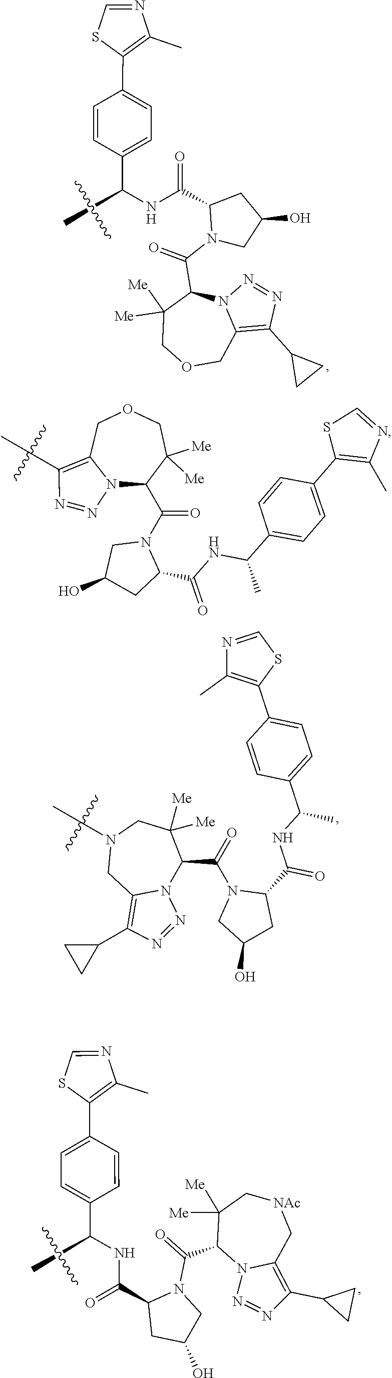

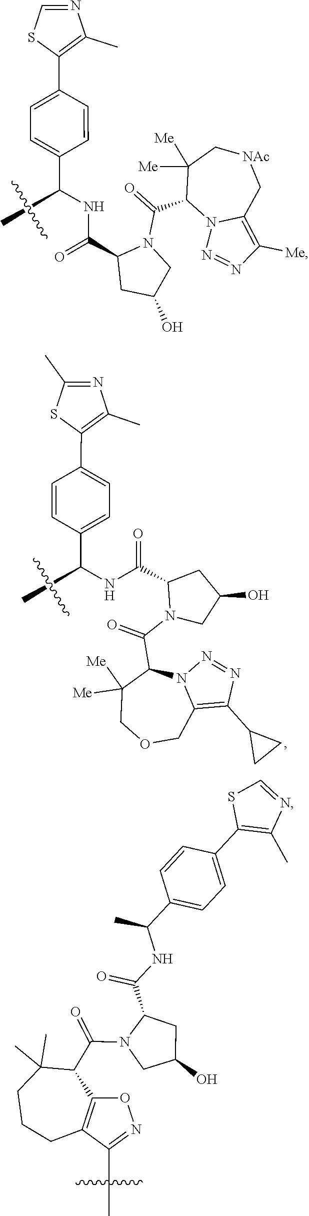

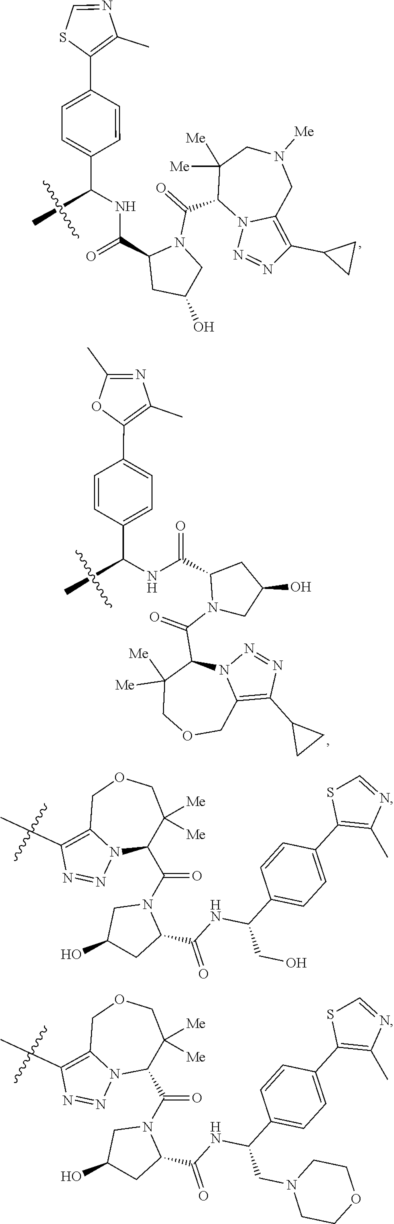

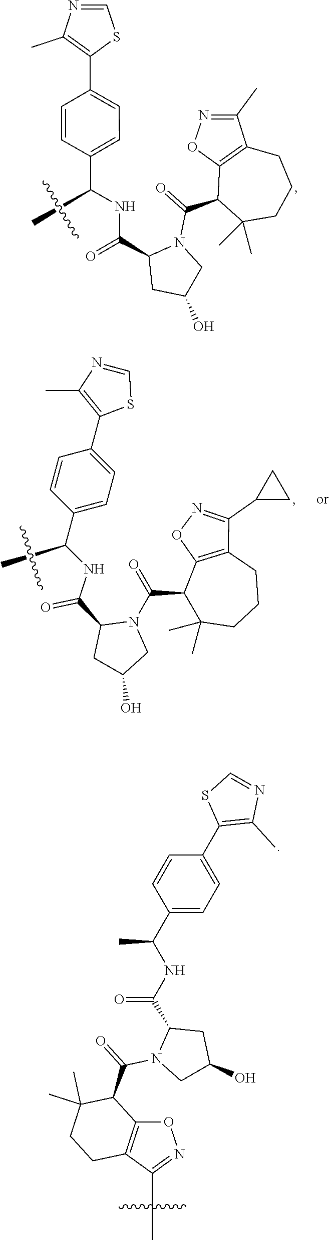

EXEMPLARY EMBODIMENT C19. A conjugate selected from the group consisting of:

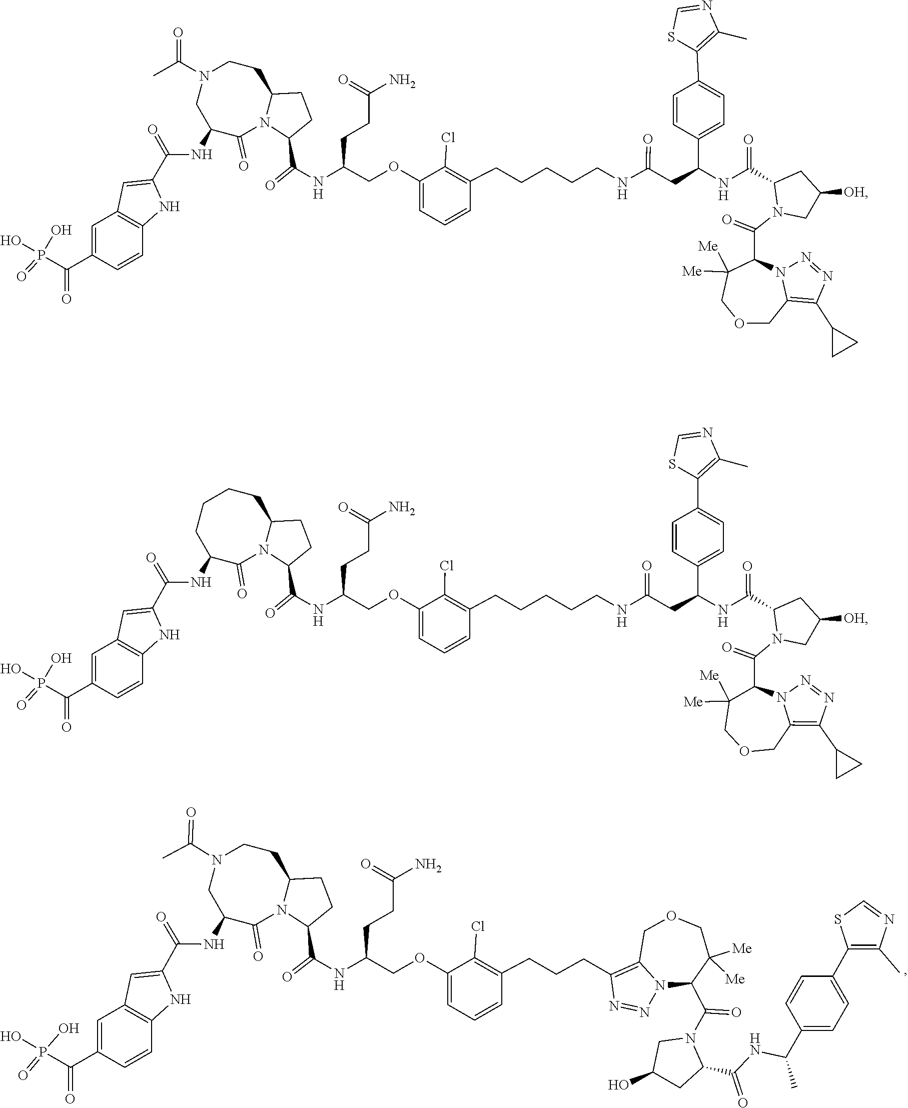

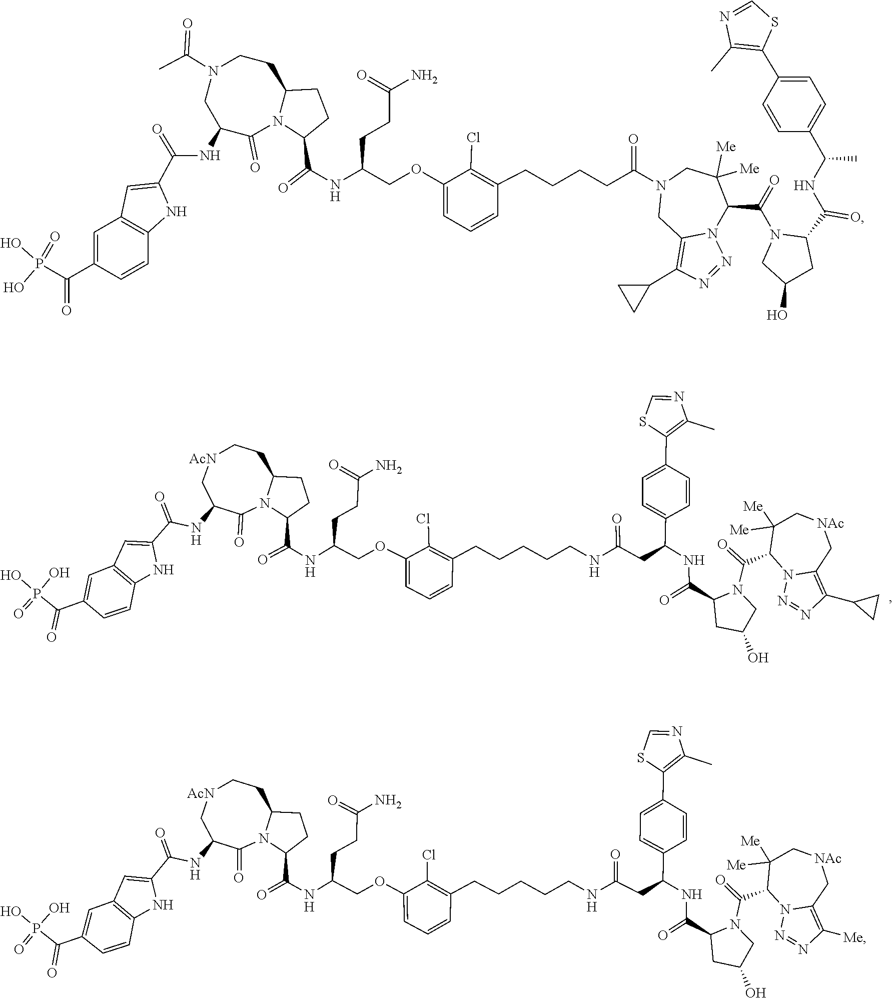

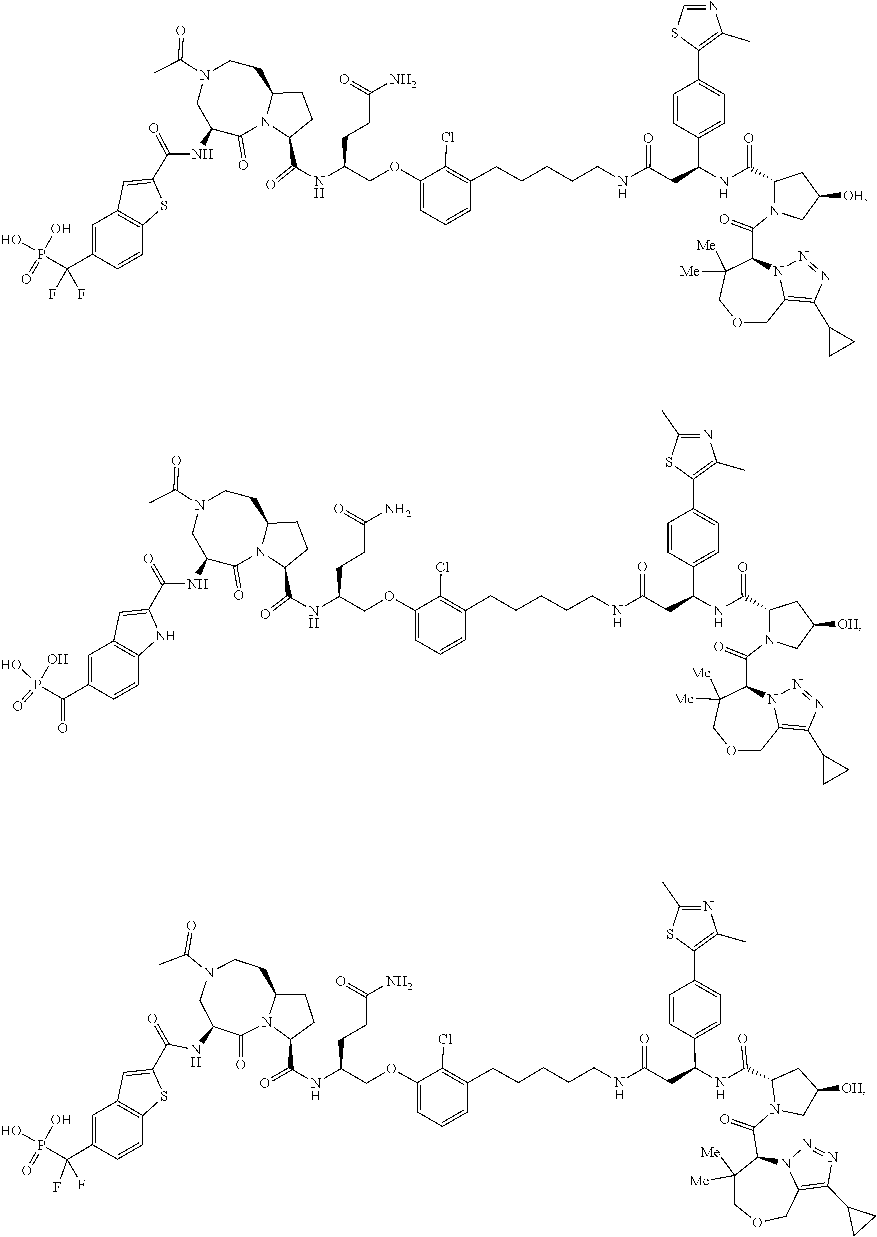

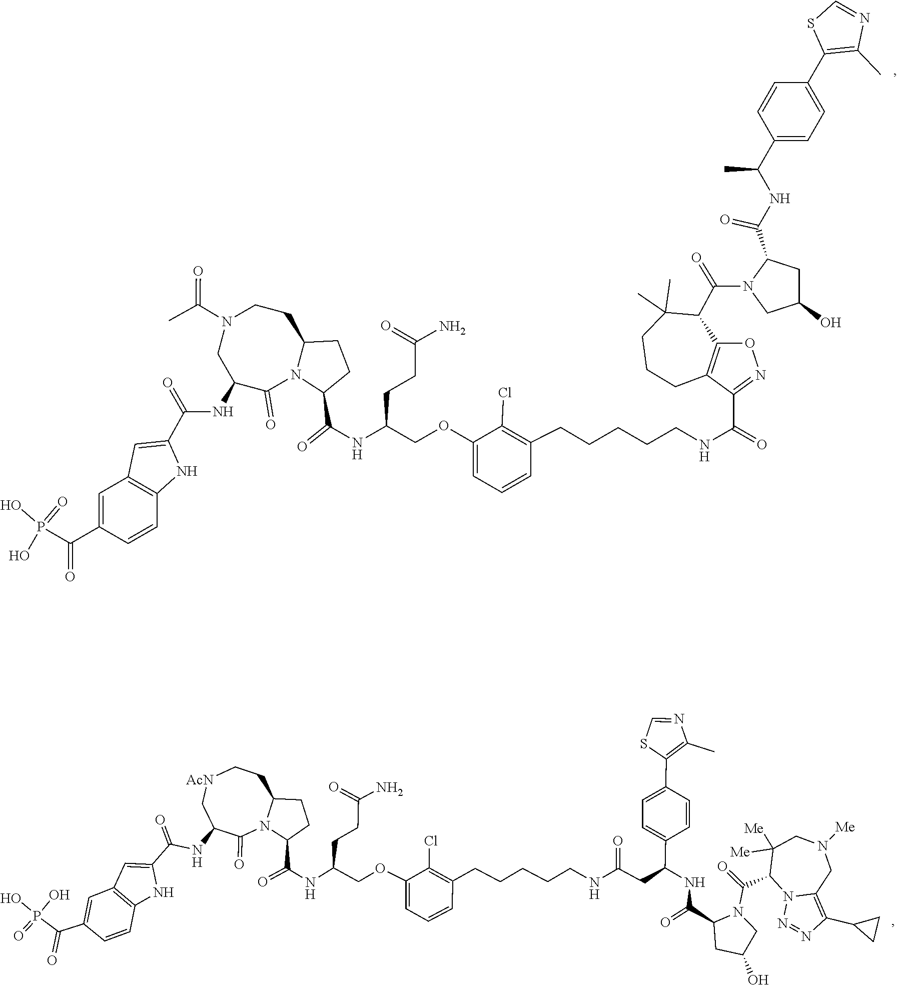

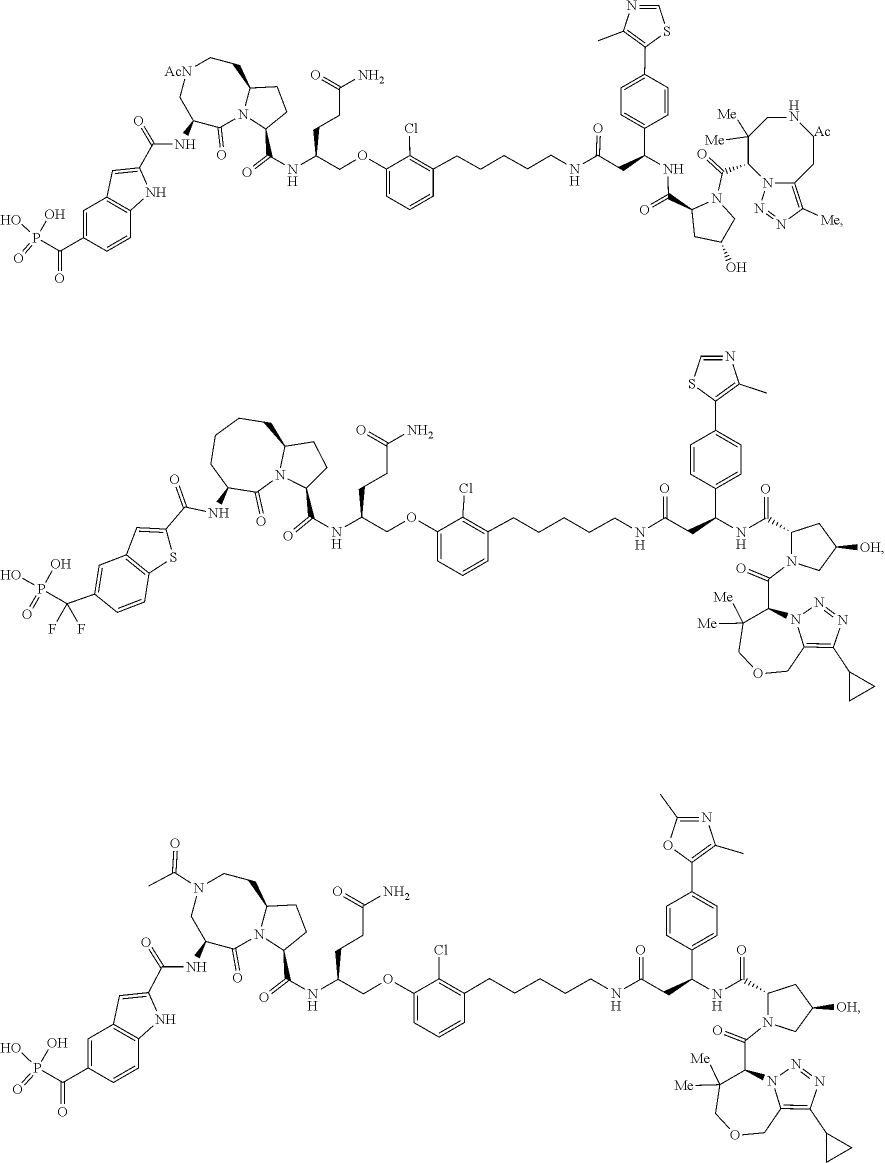

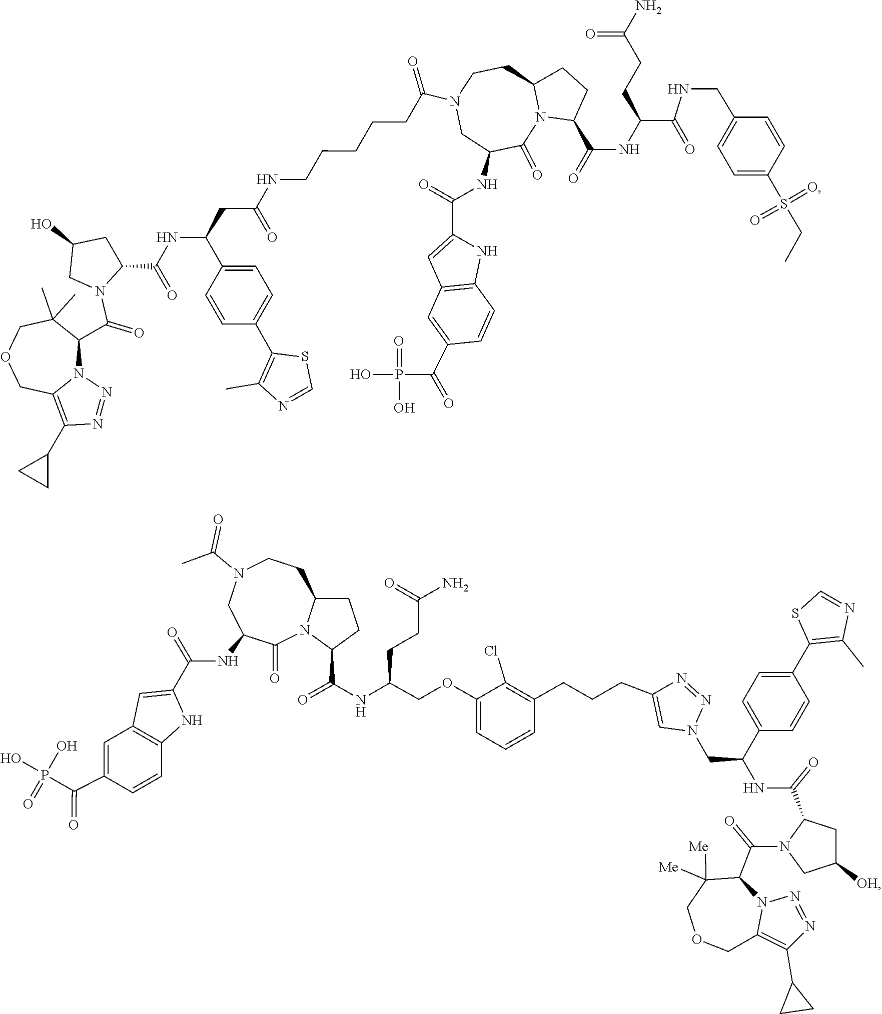 [SMILES: CCS(=O)(=O)c1ccc(CNC(=O)[C@H](CCC(N)=O)NC(=O)[C@@H]2CC[C@@H]3CCN(C(=O)CCCCCNC(=O)C[C@H](NC(=O)[C@H]4C[C@H](O)CN4C(=O)[C@H]4n5nnc(C6CC6)c5COCC4(C)C)c4ccc(-c5scnc5C)cc4)C[C@H](NC(=O)c4cc5cc(C(=O)P(=O)(O)O)ccc5[nH]4)C(=O)N32)cc1 ; CC(=O)N1CC[C@H]2CC[C@@H](C(=O)N[C@@H](CCC(N)=O)COc3cccc(CCCc4cn(C[C@H](NC(=O)[C@@H]5C[C@@H](O)CN5C(=O)[C@H]5n6nnc(C7CC7)c6COCC5(C)C)c5ccc(-c6scnc6C)cc5)nn4)c3Cl)N2C(=O)[C@@H](NC(=O)c2cc3cc(C(=O)P(=O)(O)O)ccc3[nH]2)C1]

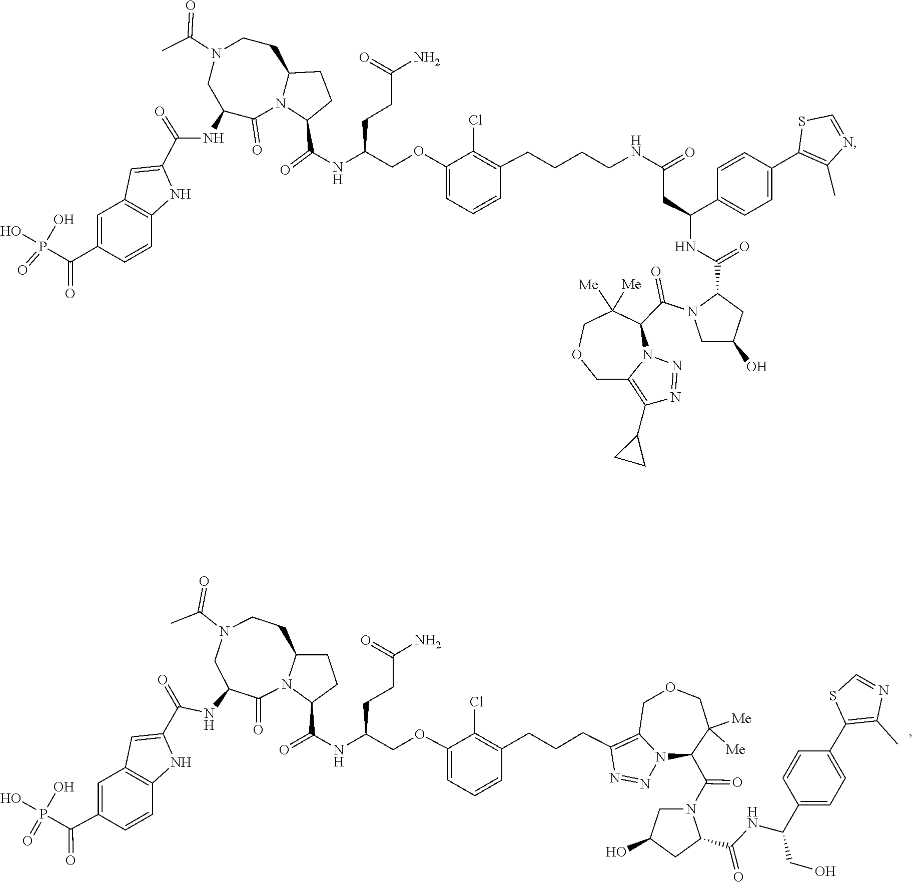 [SMILES: CC(=O)N1CC[C@H]2CC[C@@H](C(=O)N[C@@H](CCC(N)=O)COc3cccc(CCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1 ; CC(=O)N1CC[C@H]2CC[C@@H](C(=O)N[C@@H](CCC(N)=O)COc3cccc(CCCc4nnn5c4COCC(C)(C)[C@H]5C(=O)N4C[C@H](O)C[C@H]4C(=O)N[C@@H](CO)c4ccc(-c5scnc5C)cc4)c3Cl)N2C(=O)[C@@H](NC(=O)c2cc3cc(C(=O)P(=O)(O)O)ccc3[nH]2)C1]

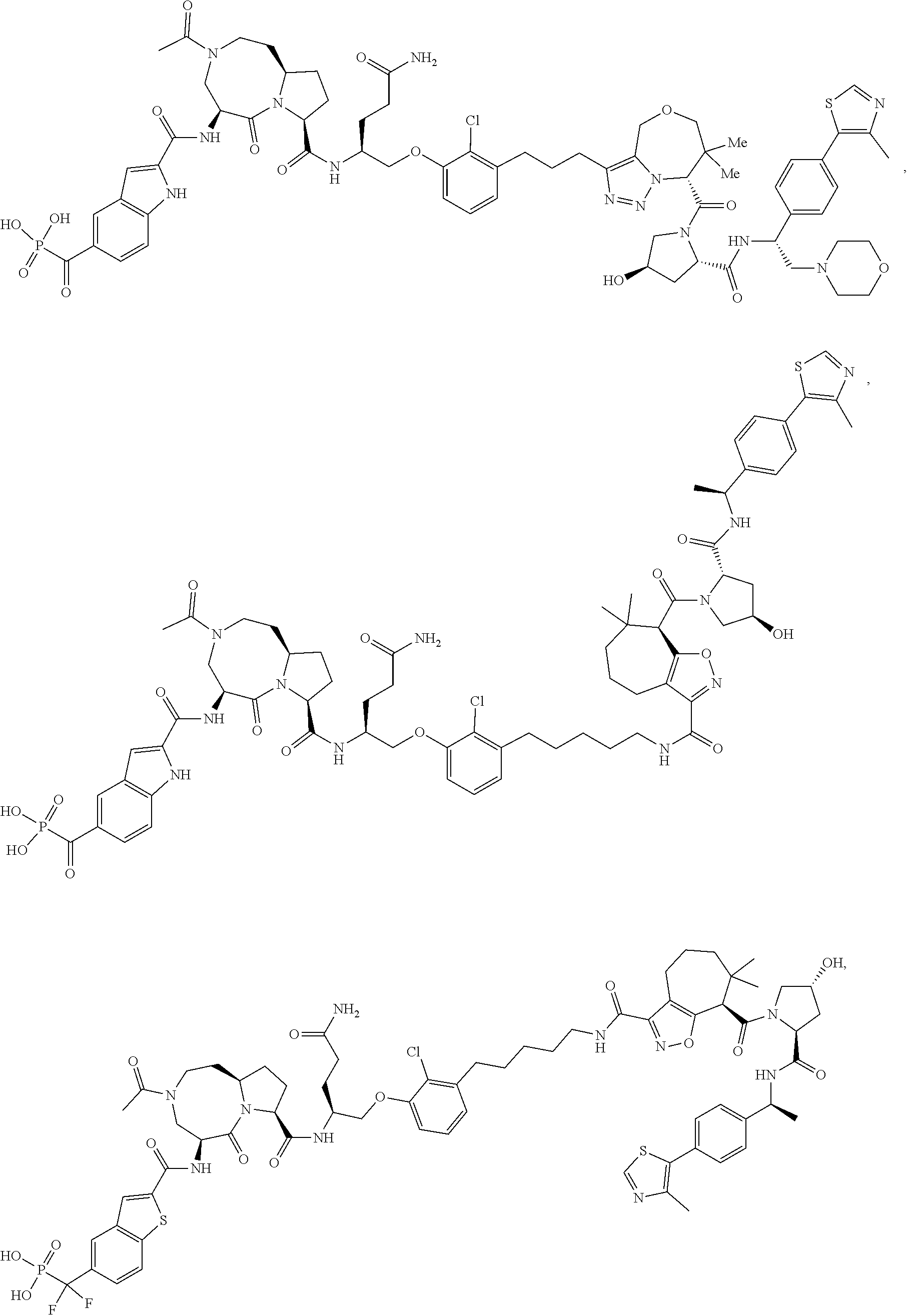

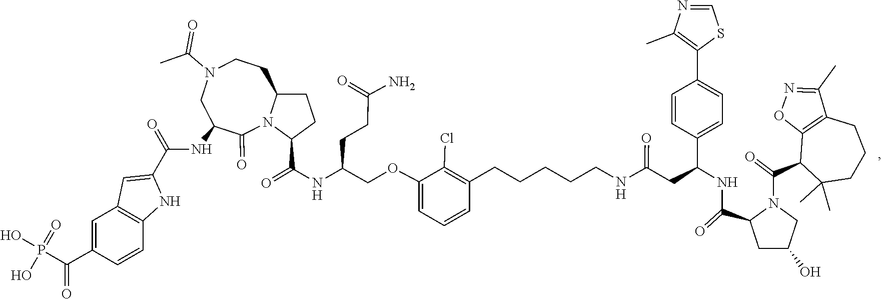 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4c5onc(C)c5CCCC4(C)C)c4ccc(-c5scnc5C)cc4)c3Cl)N2C(=O)[C@@H](NC(=O)c2cc3cc(C(=O)P(=O)(O)O)ccc3[nH]2)C1]

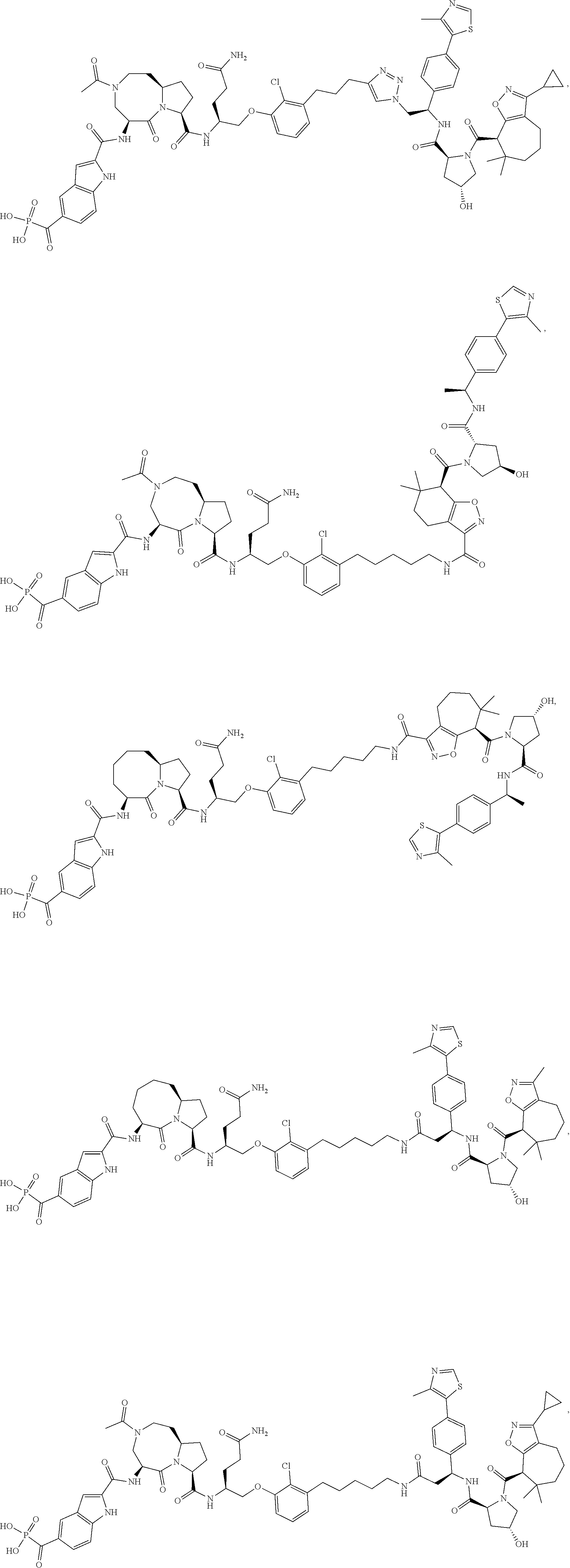

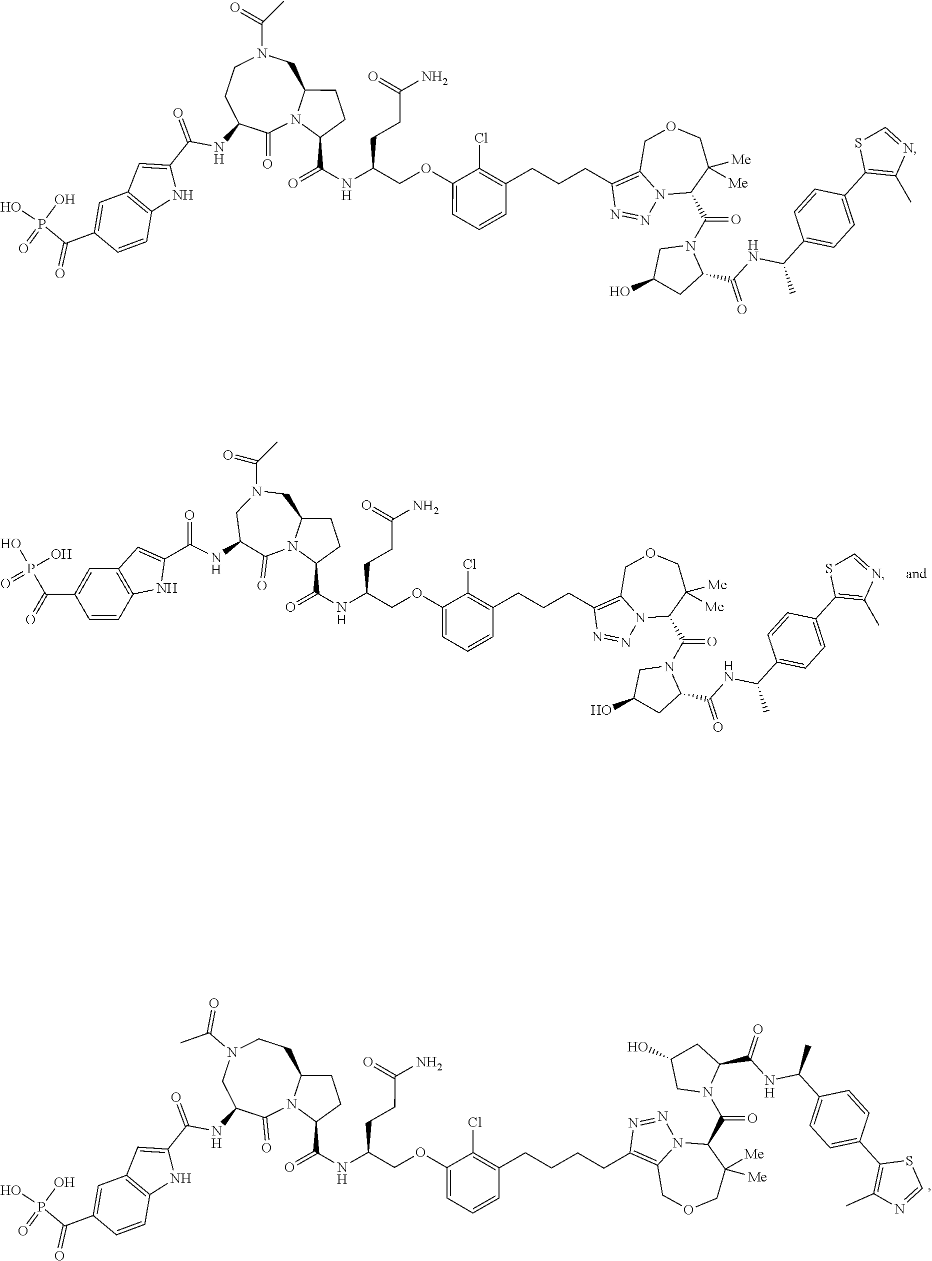

or a pharmaceutically acceptable salt thereof.

EXEMPLARY EMBODIMENT C20. The conjugate of EXEMPLARY EMBODIMENT C19, or a pharmaceutically acceptable salt thereof, that is:

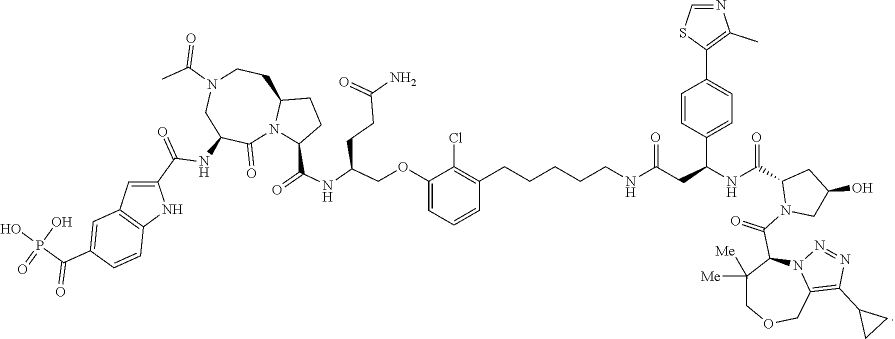 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1]

EXEMPLARY EMBODIMENT C21. The conjugate of EXEMPLARY EMBODIMENT C19, or a pharmaceutically acceptable salt thereof, that is:

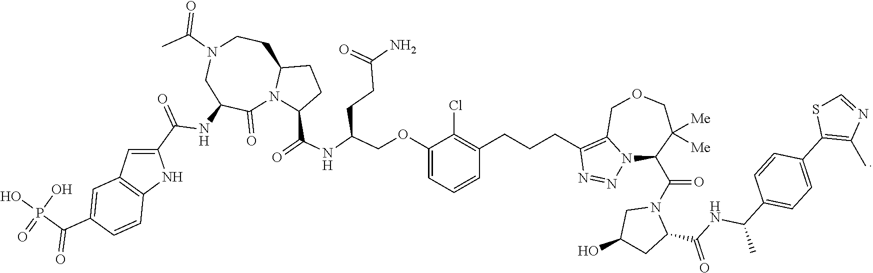 [SMILES: CC(=O)N1CC[C@H]2CC[C@@H](C(=O)N[C@@H](CCC(N)=O)COc3cccc(CCCc4nnn5c4COCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1]

EXEMPLARY EMBODIMENT C22. The conjugate of EXEMPLARY EMBODIMENT C19, or a pharmaceutically acceptable salt thereof, that is:

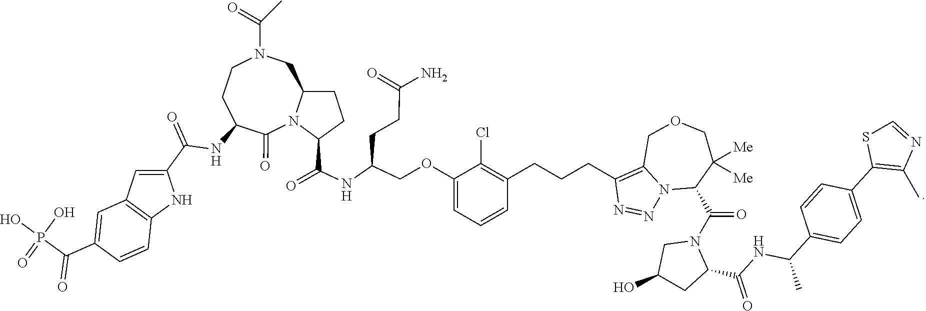 [SMILES: CC(=O)N1CC[C@H](NC(=O)c2cc3cc(C(=O)P(=O)(O)O)ccc3[nH]2)C(=O)N2[C@H](CC[C@H]2C(=O)N[C@@H](CCC(N)=O)COc2cccc(CCCc3nnn4c3COCC(C)(C)[C@@H]4C(=O)N3C[C@H](O)C[C@H]3C(=O)N[C@@H](C)c3ccc(-c4scnc4C)cc3)c2Cl)C1]

EXEMPLARY EMBODIMENT C23. A pharmaceutical composition comprising the conjugate of any one of EXEMPLARY EMBODIMENTS C1-C22, or a pharmaceutically acceptable salt thereof, and a pharmaceutically acceptable excipient.

EXEMPLARY EMBODIMENT C24. A method of treating a STAT3-mediated disease or disorder comprising administering to a patient in need thereof a conjugate of any one of EXEMPLARY EMBODIMENTS C1-C22, wherein the STAT3-mediated disease or disorder is cancer, an autoimmune disease, an inflammatory disorder, a neurodegenerative disease, a viral disease, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, or a CNS disorder.

EXEMPLARY EMBODIMENT C25. A compound of Formula I:

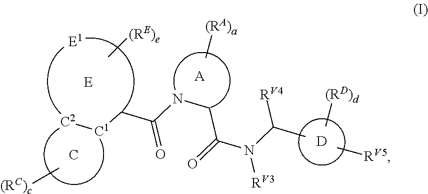

or a pharmaceutically acceptable salt, solvate, or stereoisomer thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl; C¹ and C² are independently C or N;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E}; E¹ is C(R^{E1′})₂, NR^{E1″}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —N₃, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₁₂ carbocyclyl, 3- to 12-membered heterocyclyl, C₆₋₁₀ aryl, or 5- to 10-membered heteroaryl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

EXEMPLARY EMBODIMENT C28. The compound of EXEMPLARY EMBODIMENT C23, wherein the compounds is selected from the group consisting of:

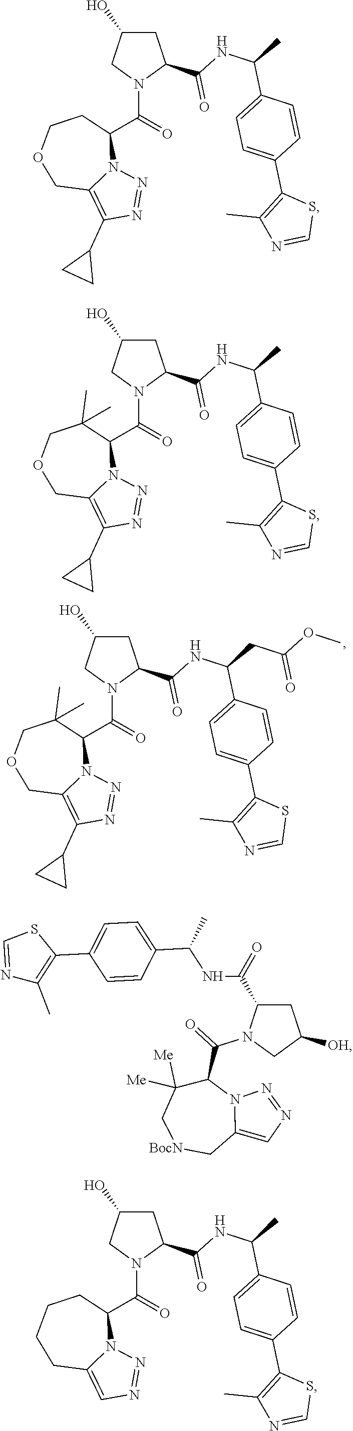

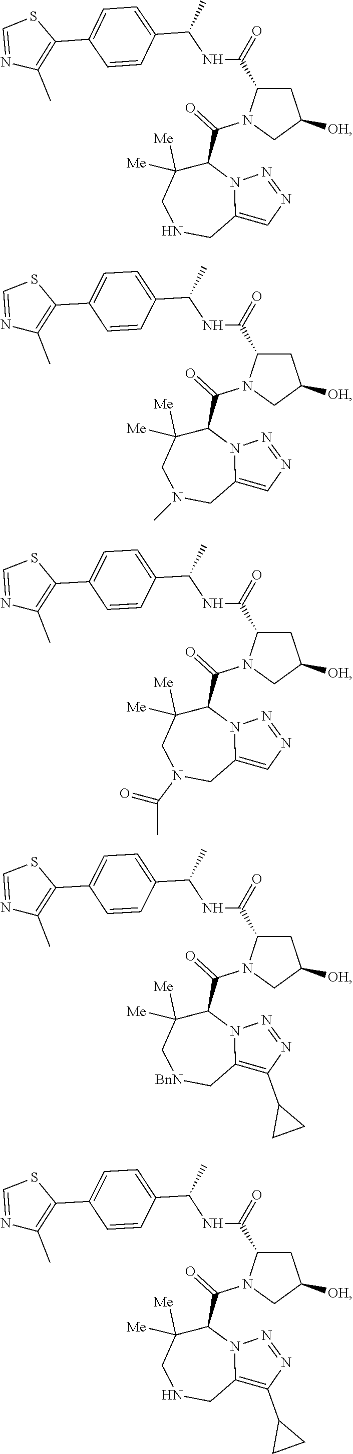

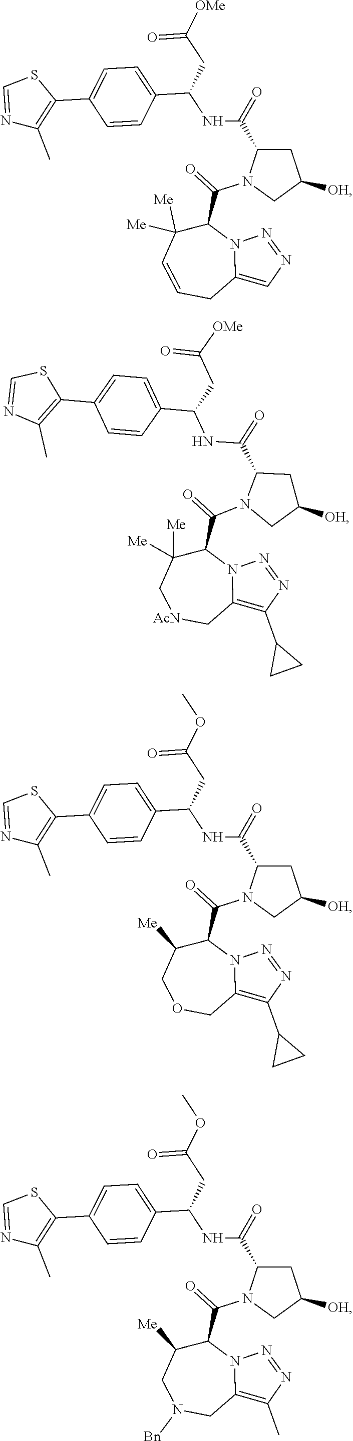

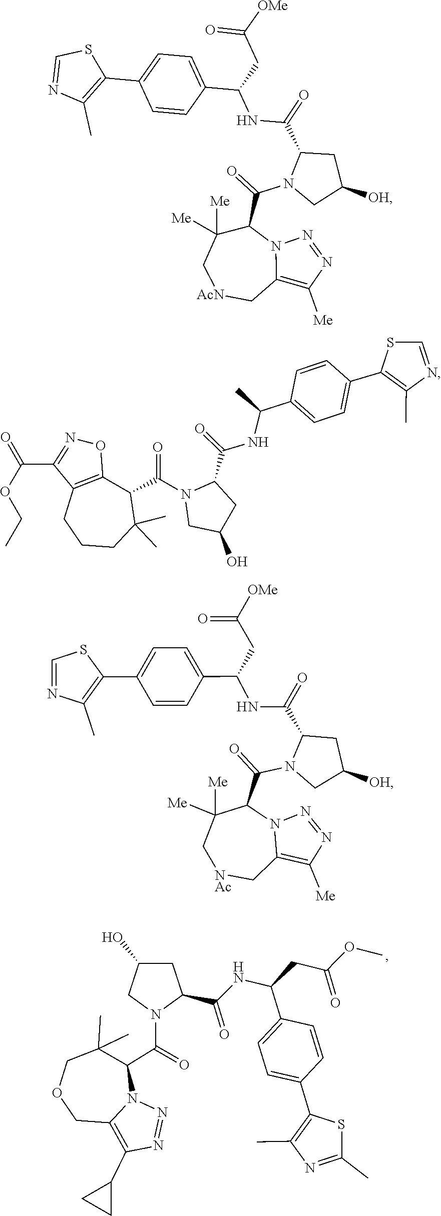

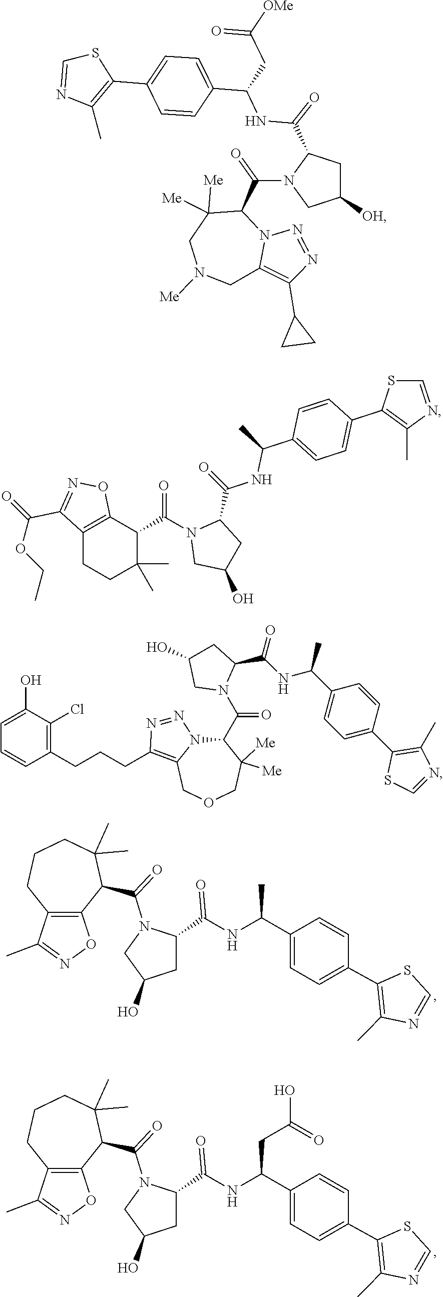

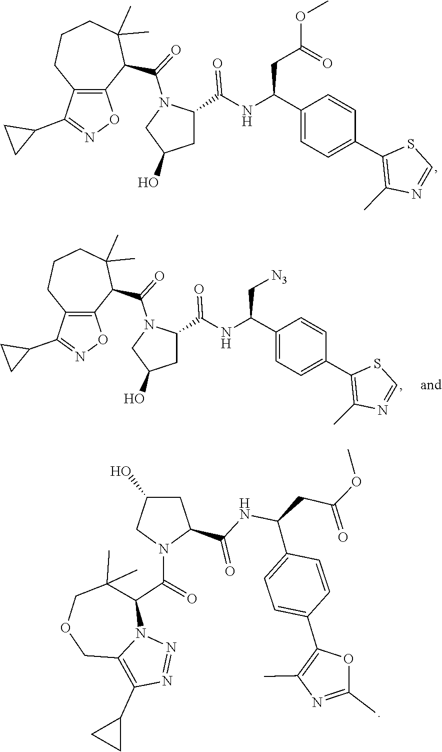

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

It is understood that, unless indicated otherwise, the values presented in the examples are approximate values, and are subject to experimental and instrumental variations.

### I. Synthesis and Characterization

### A. VHL Ligands

### Compound A1. (2S,4R)-1-(3-cyclopropyl-6,7,8,9-tetrahydro-4H-[1,2,3]triazolo[5,1-c][1,4]oxazocine-9-carbonyl)-4-hydroxy-N-((S)-1-(4-(4-methylthiazol-5-yl)phenyl)ethyl)pyrrolidine-2-carboxamide

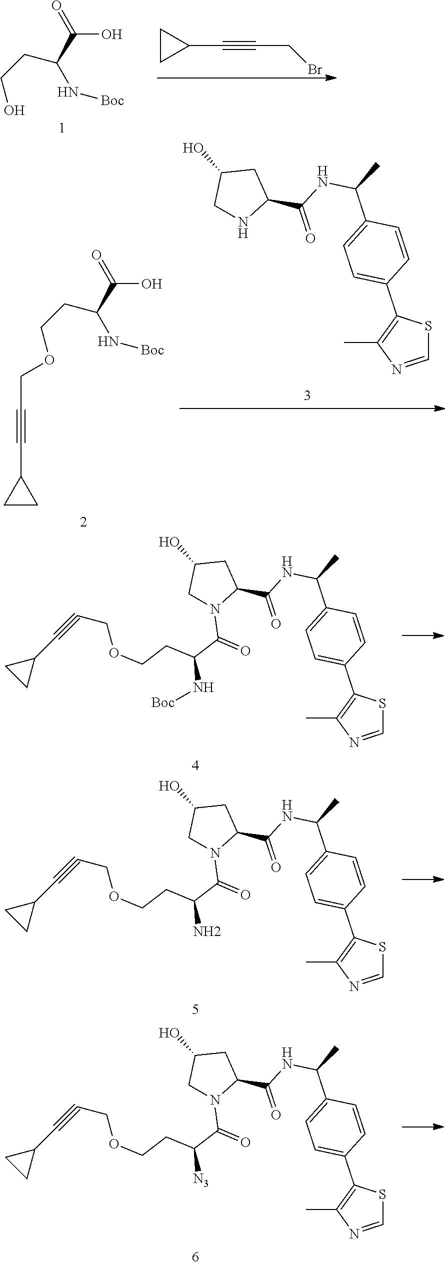

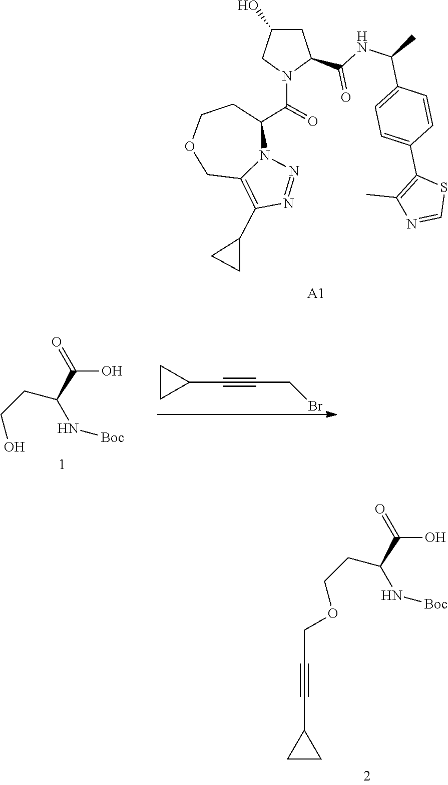 [SMILES: Cc1ncsc1-c1ccc([C@H](C)NC(=O)[C@@H]2C[C@@H](O)CN2C(=O)[C@@H]2CCOCc3c(C4CC4)nnn32)cc1 ; "A1" ; CC(C)(C)OC(=O)N[C@@H](CCO)C(=O)O ; BrCC#CC1CC1 ; CC(C)(C)OC(=O)N[C@@H](CCOCC#CC1CC1)C(=O)O]

(S)-2-((tert-butoxycarbonyl)amino)-5-hydroxypentanoic acid (1, 600 mg, 2.7 mmol, 1 equiv.) was dissolved in DMF (10 ml). The solution was cooled to 0° C. and NaH (274 mg, 6.8 mmol, 60 wt-% suspension in mineral oil, 2.5 equiv) was added in portions. After stirring for 0.5-1 h at 0° C., (3-bromoprop-1-yn-1-yl)cyclopropane was added dropwise. The reaction was slowly (1-2 h) warmed to rt and stirred at rt for 2-4 h. The reaction was quenched by addition of 1 mL water, and then acidified with 1M KHSO4 to pH 1-2. The resulting mixture was extracted with EtOAc and the combined organic phases were washed with brine. The organic layer was dried over Na₂SO₄, filtered and concentrated in vacuo to afford 2 which was used without further purification. UPLC-MS calculated [M+H]⁺: 298.2, found: 198.3. Retention time: 1.8 min.

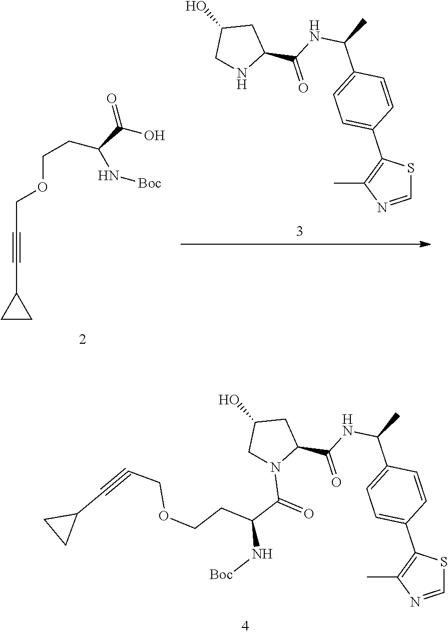 [SMILES: CC(C)(C)OC(=O)N[C@@H](CCOCC#CC1CC1)C(=O)O ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)[C@H](CCOCC#CC2CC2)NC(=O)OC(C)(C)C)cc1]

HATU (0.55 mmol, 1.1 equiv.) was added to a solution of 2 (0.5 mmol, 1 equiv.), 3 (0.5 mmol, 1 equiv.) and DIEA (1.5 mmol, 3 equiv.) in DMF (4 mL) and the resulting mixture was stirred at rt for 1 h. This mixture was purified by reverse phase preparative HPLC to give 4. UPLC-MS calculated [M+H]⁺: 611.3, found: 611.5.

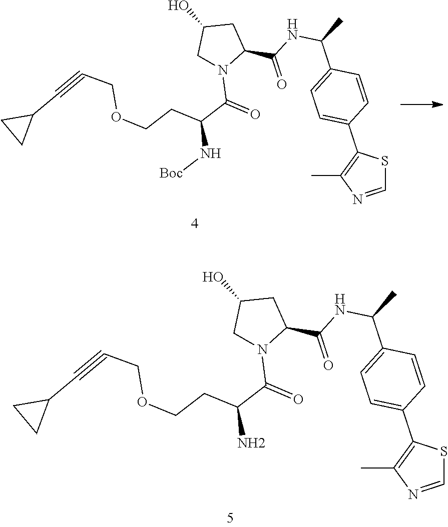 [SMILES: Cc1ncsc1-c1ccc([C@H](C)NC(=O)[C@@H]2C[C@@H](O)CN2C(=O)[C@H](CCOCC#CC2CC2)NC(=O)OC(C)(C)C)cc1 ; Cc1ncsc1-c1ccc([C@H](C)NC(=O)[C@@H]2C[C@@H](O)CN2C(=O)[C@@H](N)CCOCC#CC2CC2)cc1]

10% H₂SO₄ (5 mL) was added to a solution of compound 7 (0.2 mmol) in MeCN (1 mL). The resulting reaction solution was stirred at rt for 4 h. This mixture was purified by reverse phase preparative HPLC to give 5. UPLC-MS calculated [M+H]⁺: 511.2, found: 511.5.

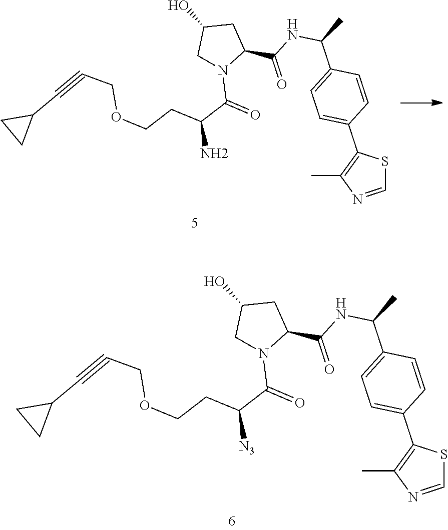 [SMILES: Cc1ncsc1-c1ccc([C@H](C)NC(=O)[C@@H]2C[C@@H](O)CN2C(=O)[C@@H](N)CCOCC#CC2CC2)cc1 ; Cc1ncsc1-c1ccc([C@H](C)NC(=O)[C@@H]2C[C@@H](O)CN2C(=O)[C@H](CCOCC#CC2CC2)N=[N+]=[N-])cc1]

1H-imidazole-1-sulfonyl azide (0.12 mmol) was added to a mixture of 5 (0.1 mmol), potassium carbonate (0.25 mmol) and cupric sulfate (0.02 mmol) in methanol (1 mL) at 25° C. The reaction mixture was stirred at 25° C. for 10 h. After removing the solvent, 10% potassium bisulfate in water (1 mL) was added and extracted with ethyl acetate. The organic layer was washed with brine, and dried over sodium sulfate. The organic layer was concentrated, and the residue was purified by reverse phase preparative HPLC to give 6. UPLC-MS calculated [M+H]⁺: 537.2, found: 537.7. Retention time: 3.6 min.

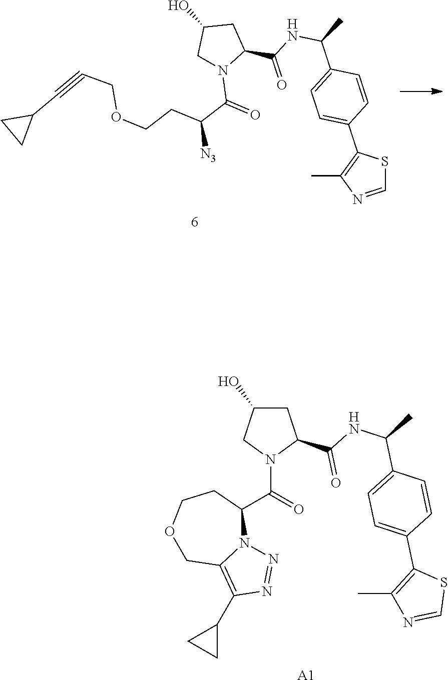 [SMILES: Cc1ncsc1-c1ccc([C@H](C)NC(=O)[C@@H]2C[C@@H](O)CN2C(=O)[C@H](CCOCC#CC2CC2)N=[N+]=[N-])cc1 ; Cc1ncsc1-c1ccc([C@H](C)NC(=O)[C@@H]2C[C@@H](O)CN2C(=O)[C@@H]2CCOCc3c(C4CC4)nnn32)cc1 ; "A1"]

To a solution of 6 (50 mg) in 1,2-dichlorobenzene (10 mL). Microwave the reaction at 140° C. for 16 h. Removal of the solvent under vacuum and the residue was purified by HPLC to yield A1. the next step without further purification. UPLC-MS calculated [M+H]²⁺: 537.2, found: 537.7. Retention time: 2.5 min.

### Compound A2. (2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxy-N-((S)-1-(4-(4-methylthiazol-5-yl)phenyl)ethyl)pyrrolidine-2-carboxamide

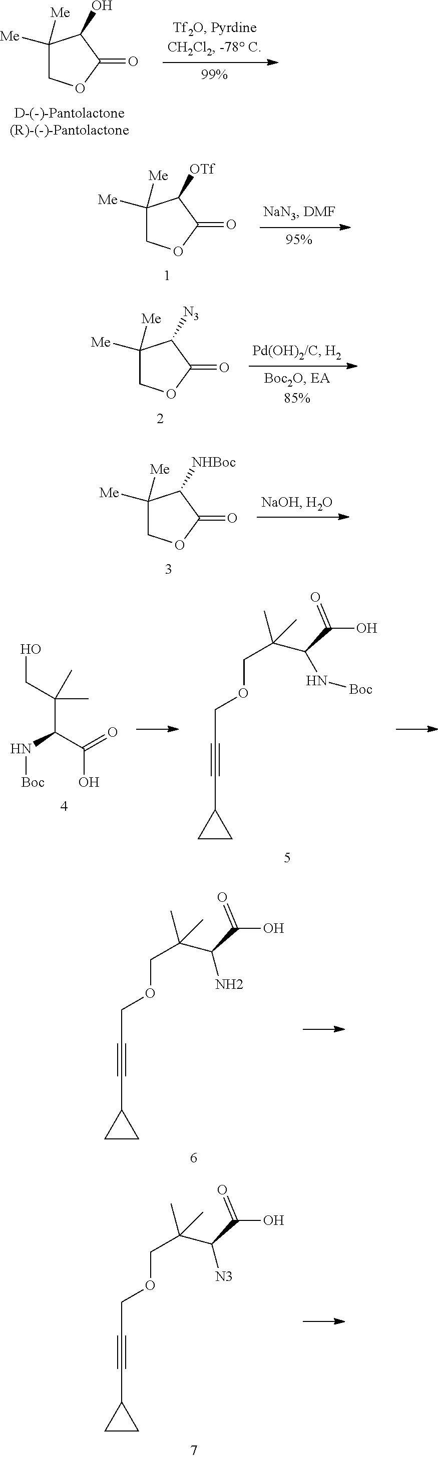

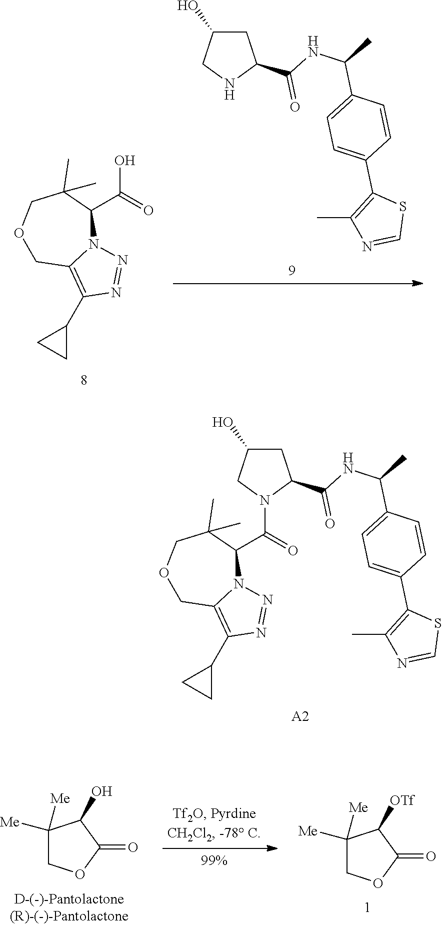 [SMILES: CC1(C)COCc2c(C3CC3)nnn2[C@@H]1C(=O)O ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)[C@H]2n3nnc(C4CC4)c3COCC2(C)C)cc1 ; "A2" ; CC1(C)COC(=O)[C@@H]1O ; CC1(C)COC(=O)[C@@H]1OS(=O)(=O)C(F)(F)F]

To a solution of (R)-(−)-Pantolactone (10 g, 1 equiv.) in 4 mL of dry dichloromethane was added pyridine (7.8 mL, 1.25 equiv.). The resulting solution was cooled to −78° C. Triflic anhydride (13.9 mL, 1.1 equiv.) was added dropwise via a syringe over 10 min. The reaction mixture was stirred at −78° C. for 30 min and then 1 h at room temperature. The reaction mixture was partitioned between 200 mL of diethyl ether and 100 mL of 20% HCl. The organic layer was washed once with 50 mL of saturated NaHCO₃, followed by 50 mL of brine. It was dried over MgSO₄ and concentrated in vacuum to afford 20 g 1 (99%), which was used in subsequent step without further purification. Rf₁=0.5, hexane/ethyl acetate=7:3.

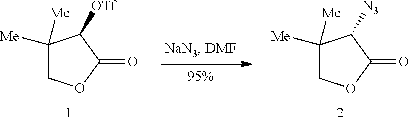 [SMILES: CC1(C)COC(=O)[C@@H]1OS(=O)(=O)C(F)(F)F ; CC1(C)COC(=O)[C@H]1N=[N+]=[N-]]

The crude product 1 (20 g, 1 equiv.) was dissolved in 50 mL of DMF. To the resulting solution was added NaN₃ (5.7 g, 1.15 equiv.). The mixture was stirred at room temperature for 2 h. The reaction mixture was poured into water (200 mL). The aqueous mixture was extracted twice with 100 mL of diethyl ether. The combined organic extract was washed once with brine (50 mL). The organic layer was dried over MgSO₄. Concentrated in vacuum and purified by silica gel flash chromatography to afford 11.3 g white powder 2 (95%). Rf₂=0.26, hexane/ethyl acetate=8:2

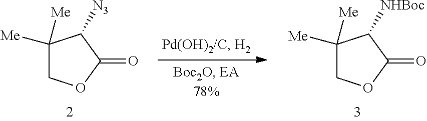 [SMILES: CC1(C)COC(=O)[C@H]1N=[N+]=[N-] ; CC(C)(C)OC(=O)N[C@@H]1C(=O)OCC1(C)C]

To a solution of 2 (2 g, 1 equiv.) in ethyl acetate (70 mL). Boc₂O (5.6 g, 2 equiv.) and Pd(OH)₂/C (0.2 g) were added into the flask. Hydrogenation with H₂ balloon for 24 h. Rf₂=0.71, Rf₃=0.43 Dichloromethane. Pd(OH)₂/C was filtered by Cilite. Concentrated in vacuum and purified by silica gel flash chromatography to afford 2.5 g white powder 3 (84.6%). UPLC-MS: calculated [M+Na]⁺: 252.12, found: 252.13.

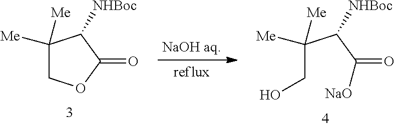

To a solution of 3 (150 mg, 1 equiv.) in NaOH aq. (5.2 mL, C_{NaOH}=0.125 mol/L, 1 equiv). The mixture was refluxed overnight. Once the reaction was detected be done by HPLC-MS, remove the water by freeze drying in vacuum to afford 4, which was used in subsequent step without further purification. UPLC-MS: calculated [M+H]⁺: 248.15, found: 247.99.

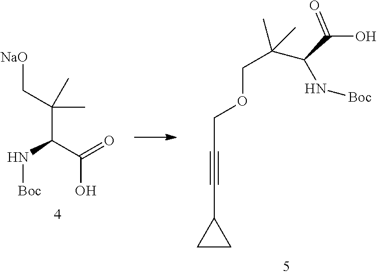

Sodium (S)-3-((tert-butoxycarbonyl)amino)-3-carboxy-2,2-dimethylpropan-1-olate (4, 100 mg, 0.37 mmol, 1 equiv.) was dissolved in DMF (3 ml). The solution was cooled to 0° C. and NaH (23 mg, 0.56 mmol, 60 wt-% suspension in mineral oil, 1.5 equiv) was added in portions. After stirring for 0.5-1 h at 0° C., (3-bromoprop-1-yn-1-yl)cyclopropane was added dropwise.

The reaction was slowly (1-2 h) warmed to rt and stirred at rt for 2-4 h. The reaction was quenched by addition of 1 mL water, and then acidified with 1M KHSO4 to pH 1-2. The resulting mixture was extracted with EtOAc and the combined organic phases were washed with brine. The organic layer was dried over Na₂SO₄, filtered and concentrated in vacuo to afford crude 5 which can be purified by HPLC to give 5. UPLC-MS calculated [M+H]⁺: 326.2, found: 270.3, 248.4, 174.2. Retention time: 4.6 min.

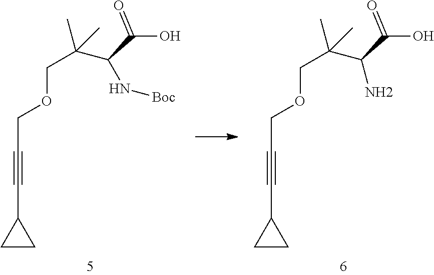 [SMILES: CC(C)(C)OC(=O)N[C@H](C(=O)O)C(C)(C)COCC#CC1CC1 ; CC(C)(COCC#CC1CC1)[C@H](N)C(=O)O]

10% H₂SO₄ (2 mL) was added to a solution of compound 5 (0.1 mmol) in MeCN (0.5 mL, for solubility). and the resulting reaction solution was stirred at rt for overnight. This mixture was purified by reverse phase preparative HPLC to give 6. UPLC-MS calculated [M+H]⁺: 226.1, found: 226.3. Retention time: 1.3 min. (Note: If TFA or HCl was used as acid to remove the Boc group, byproduct was formed by attacking on the triple bond. This is why H₂SO₄ was used.)

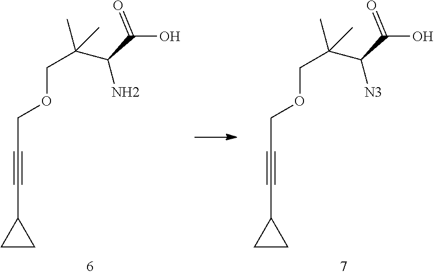 [SMILES: CC(C)(COCC#CC1CC1)[C@H](N)C(=O)O ; CC(C)(COCC#CC1CC1)[C@H](N=[N+]=[N-])C(=O)O]

1H-imidazole-1-sulfonyl azide (0.13 mmol) was added to a mixture of 6 (0.1 mmol), DIEA (0.4 mmol) and cupric sulfate (0.02 mmol) in methanol (1 mL) at 25° C. The reaction mixture was stirred at 25° C. for 0.5-1 h. Purification by reverse phase preparative HPLC gave 7. UPLC-MS calculated [M+H]⁺: 252.2, found: 252.3. Retention time: 3.8 min.

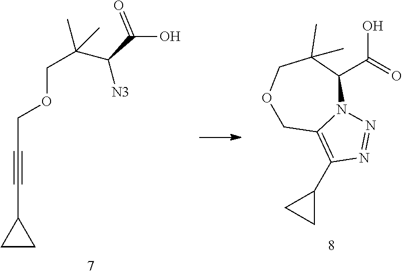 [SMILES: CC(C)(COCC#CC1CC1)[C@H](N=[N+]=[N-])C(=O)O ; CC1(C)COCc2c(C3CC3)nnn2[C@@H]1C(=O)O]

A solution of 7 (30 mg) in 1,2-dichlorobenzene (10 mL) was stirred at 140° C. by microwave for 16 h. Removal of the solvent under vacuum yielded the crude product 10 which was used for the next step without further purification. UPLC-MS calculated [M+H]⁺: 252.1, found: 252.1. Retention time: 1.7 min.

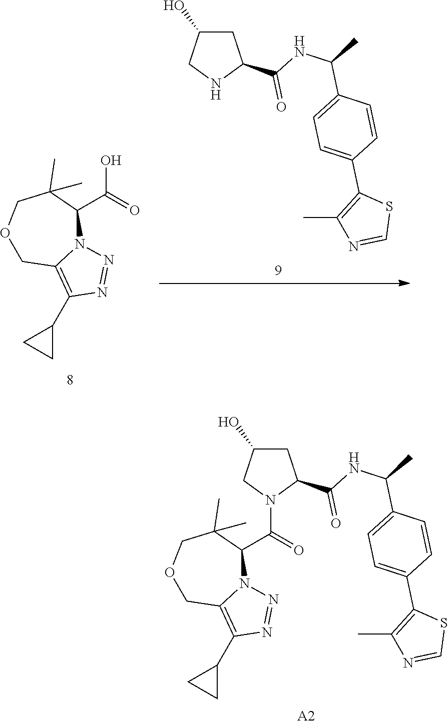 [SMILES: CC1(C)COCc2c(C3CC3)nnn2[C@@H]1C(=O)O ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)[C@H]2n3nnc(C4CC4)c3COCC2(C)C)cc1 ; "A2"]

HATU (0.08 mmol, 1.1 equiv.) was added to a solution of 8 (18 mg, 0.07 mmol, 1 equiv.), 9 (0.07 mmol, 1 equiv.) and DIEA (0.21 mmol, 3 equiv.) in DMF (1 mL) and the resulting mixture was stirred at rt for 1 h. This mixture was purified by reverse phase preparative HPLC to give A2. UPLC-MS calculated [M+H]⁺: 565.3, found: 565.6. Retention time: 3.6 min.

### Compound A3. methyl (S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanoate

### Scheme 1:

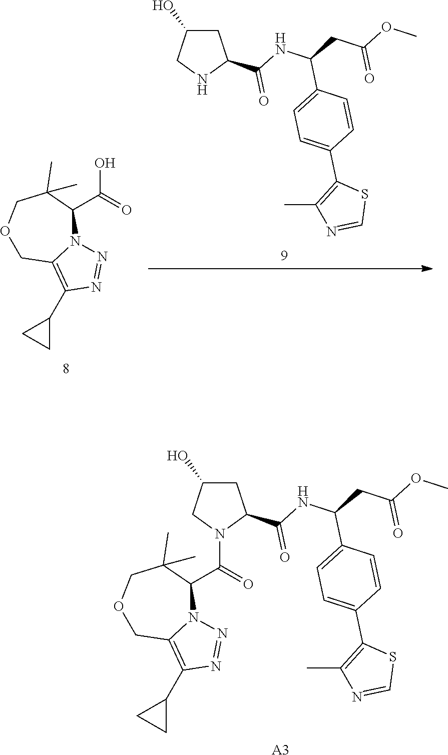 [SMILES: CC1(C)COCc2c(C3CC3)nnn2[C@@H]1C(=O)O ; COC(=O)C[C@H](NC(=O)[C@@H]1C[C@@H](O)CN1)c1ccc(-c2scnc2C)cc1 ; COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; "A3"]

HATU (0.08 mmol, 1.1 equiv.) was added to a solution of 8 (18 mg, 0.07 mmol, 1 equiv.), 9 (0.07 mmol, 1 equiv.) and DIEA (0.21 mmol, 3 equiv.) in DMF (1 mL) and the resulting mixture was stirred at rt for 1 h. This mixture was purified by reverse phase preparative HPLC to give ZH-7. UPLC-MS calculated [M+H]⁺: 623.3, found: 623.5. Retention time: 3.2 min.

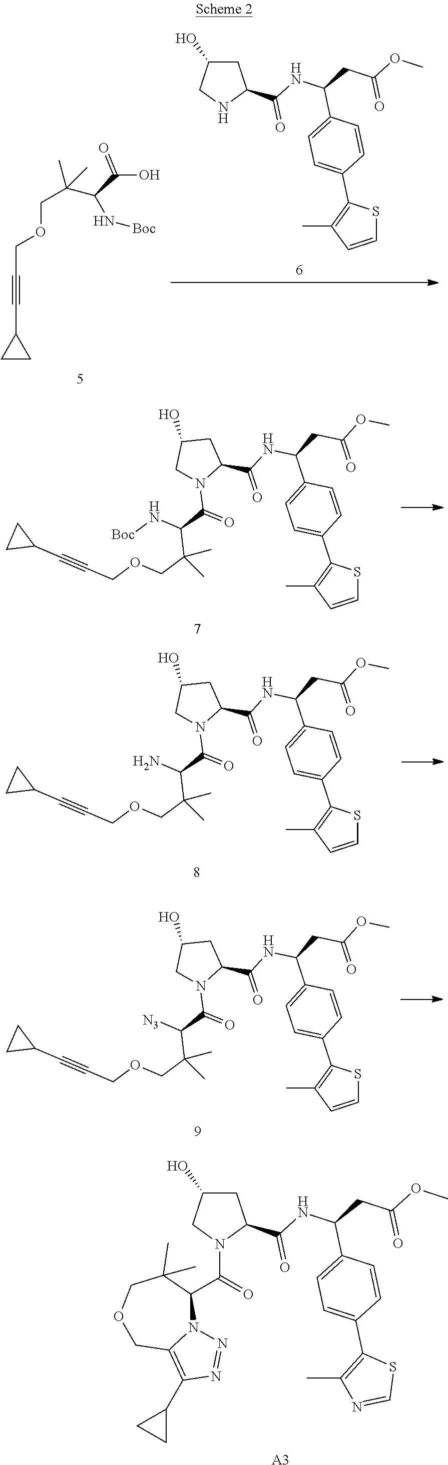

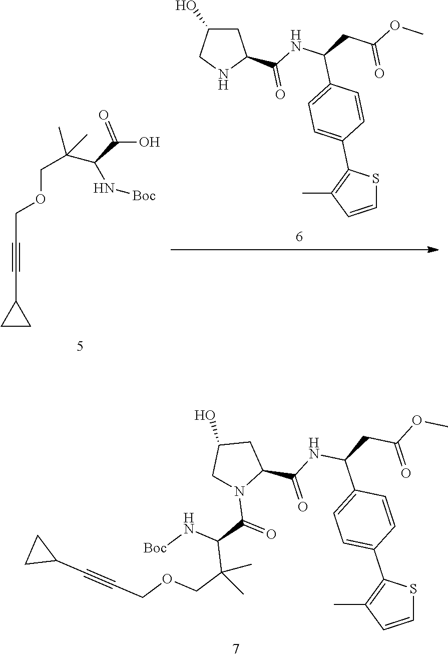 [SMILES: CC(C)(C)OC(=O)N[C@H](C(=O)O)C(C)(C)COCC#CC1CC1 ; COC(=O)C[C@H](NC(=O)[C@@H]1C[C@@H](O)CN1)c1ccc(-c2sccc2C)cc1 ; COC(=O)C[C@H](NC(=O)[C@@H]1C[C@@H](O)CN1C(=O)[C@H](NC(=O)OC(C)(C)C)C(C)(C)COCC#CC1CC1)c1ccc(-c2sccc2C)cc1]

HATU (0.18 mmol, 1.1 equiv.) was added to a solution of 5 (0.16 mmol, 1 equiv.), 6 (0.16 mmol, 1 equiv.) and DIEA (0.48 mmol, 3 equiv.) in DMF (2 mL) and the resulting mixture was stirred at rt for 1 h. This mixture was purified by reverse phase preparative HPLC to give 7. UPLC-MS calculated [M+H]⁺: 697.3, found: 697.5. Retention time: 5.2 min.

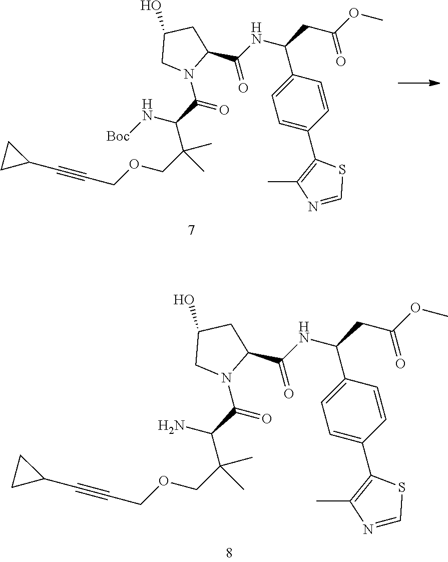 [SMILES: COC(=O)C[C@H](NC(=O)[C@@H]1C[C@@H](O)CN1C(=O)[C@H](NC(=O)OC(C)(C)C)C(C)(C)COCC#CC1CC1)c1ccc(-c2scnc2C)cc1 ; COC(=O)C[C@H](NC(=O)[C@@H]1C[C@@H](O)CN1C(=O)[C@H](N)C(C)(C)COCC#CC1CC1)c1ccc(-c2scnc2C)cc1]

10% H2SO4 was added to a solution of compound 7 (0.05 mmol) in MeCN (0.5 mL). and the resulting reaction solution was stirred at rt for 4 h. This mixture was purified by reverse phase preparative HPLC to give 8. UPLC-MS calculated [M+H]⁺: 597.3, found: 597.8. Retention time: 3.3 min.

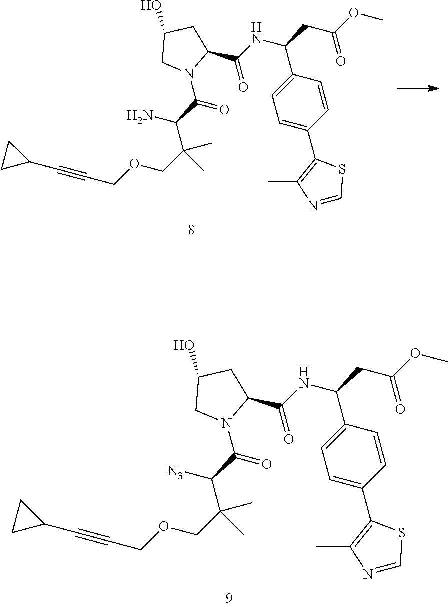 [SMILES: COC(=O)C[C@H](NC(=O)[C@@H]1C[C@@H](O)CN1C(=O)[C@H](N)C(C)(C)COCC#CC1CC1)c1ccc(-c2scnc2C)cc1 ; COC(=O)C[C@H](NC(=O)[C@@H]1C[C@@H](O)CN1C(=O)[C@H](N=[N+]=[N-])C(C)(C)COCC#CC1CC1)c1ccc(-c2scnc2C)cc1]

1H-imidazole-1-sulfonyl azide (0.12 mmol) was added to a mixture of 8 (0.1 mmol), potassium carbonate (0.25 mmol) and cupric sulfate (0.02 mmol) in methanol (1 mL) at 25° C. The reaction mixture was stirred at 25° C. for 10 h. After removing the solvent, 10% potassium bisulfate in water (1 mL) was added and extracted with ethyl acetate. The organic layer was washed with brine, and dried over sodium sulfate. The organic layer was concentrated, and the residue was purified by reverse phase preparative HPLC to give 9. UPLC-MS calculated [M+H]⁺: 623.3, found: 623.5. Retention time: 4.8 min.

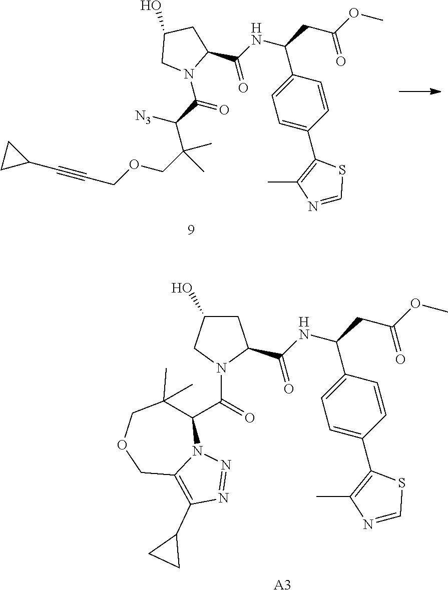 [SMILES: COC(=O)C[C@H](NC(=O)[C@@H]1C[C@@H](O)CN1C(=O)[C@H](N=[N+]=[N-])C(C)(C)COCC#CC1CC1)c1ccc(-c2scnc2C)cc1 ; COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; "A3"]

To a solution of 9 (30 mg) in 1,2-dichlorobenzene (1 mL). Microwave the reaction at 140° C. for 16 h. Removal of the solvent under vacuum to yield the crude product A3 which was used for the next step without further purification. UPLC-MS calculated [M+H]⁺: 623.3, found: 623.5. Retention time: 3.2 min.

### Compound A4. tert-butyl (S)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-7,8-dihydro-4H-[1,2,3]triazolo[1,5-a][1,4]diazepine-5 (6H)-carboxylate

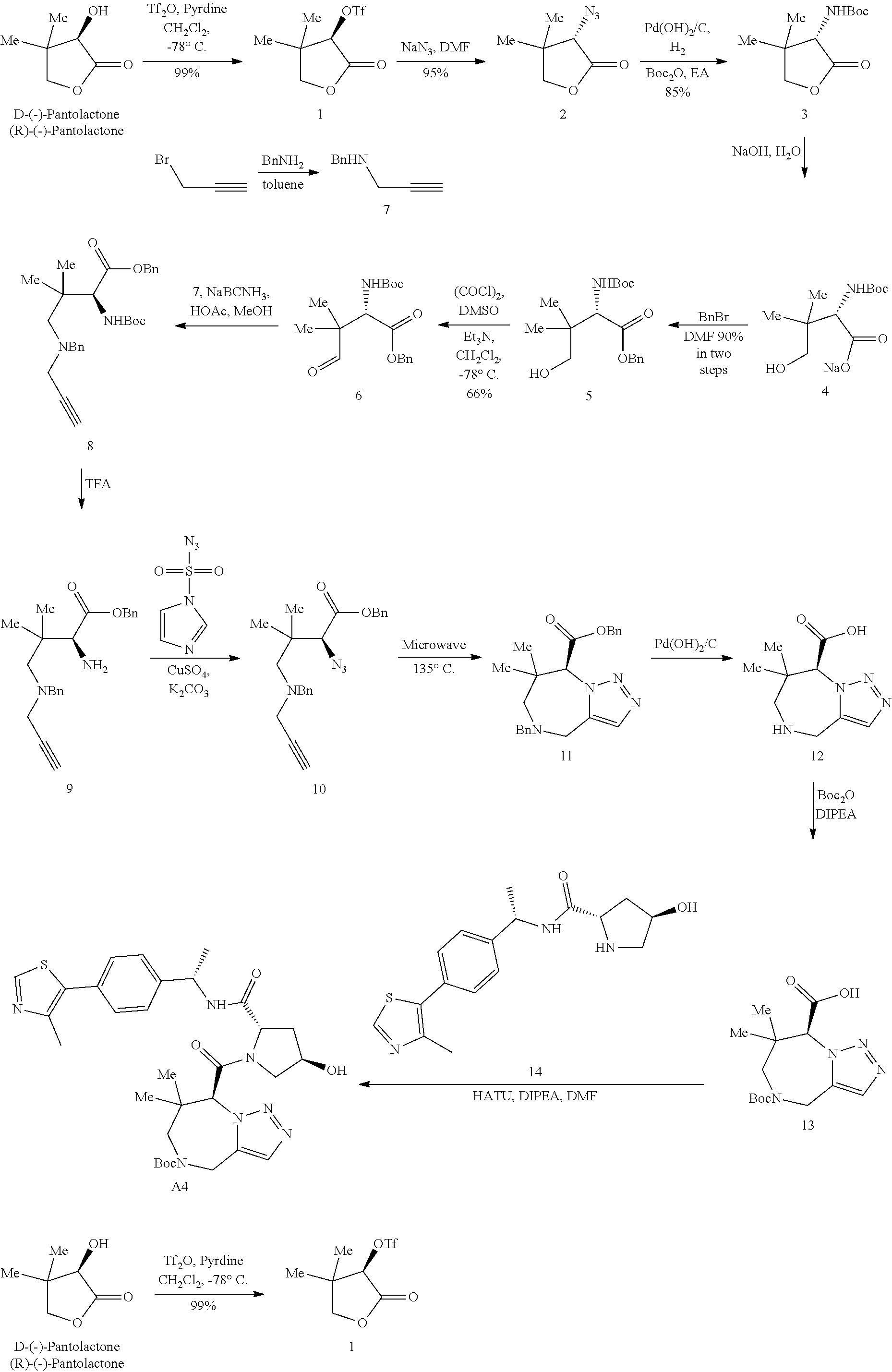

To a solution of (R)-(−)-Pantolactone (10 g, 1 equiv.) in 4 mL of dry dichloromethane was added pyridine (7.8 mL, 1.25 equiv.). The resulting solution was cooled to −78° C. Triflic anhydride (13.9 mL, 1.1 equiv.) was added dropwise via a syringe over 10 min. The reaction mixture was stirred at −78° C. for 30 min and then 1 h at room temperature. The reaction mixture was partitioned between 200 mL of diethyl ether and 100 mL of 20% HCl. The organic layer was washed once with 50 mL of saturated NaHCO₃, followed by 50 mL of brine. It was dried over MgSO₄ and concentrated in vacuum to afford 20 g 1 (99%), which was used in subsequent step without further purification. Rf₁=0.5, hexane/ethyl acetate=7:3.

¹H-NMR (400 MHz, CDCl₃) δ: 5.07 (S, 3H), 4.15-4.05 (m, 2H), 1.31 (s, 3H), 1.23 (s, 3H).

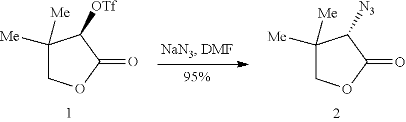 [SMILES: CC1(C)COC(=O)[C@@H]1OS(=O)(=O)C(F)(F)F ; CC1(C)COC(=O)[C@H]1N=[N+]=[N-]]

The crude product 1 (20 g, 1 equiv.) was dissolved in 50 mL of DMF. To the resulting solution was added NaN₃ (5.7 g, 1.15 equiv.). The mixture was stirred at room temperature for 2 h. The reaction mixture was poured into water (200 mL). The aqueous mixture was extracted twice with 100 mL of diethyl ether. The combined organic extract was washed once with brine (50 mL). The organic layer was dried over MgSO₄. Concentrated in vacuum and purified by silica gel flash chromatography to afford 11.3 g white powder 2 (95%). Rf₂=0.26, hexane/ethyl acetate=8:2

¹H-NMR (400 MHz, CDCl₃) δ: 4.04-3.92 (m, 3H), 1.23 (s, 3H), 1.1 (s, 3H).

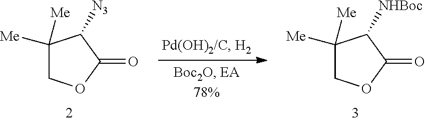 [SMILES: CC1(C)COC(=O)[C@H]1N=[N+]=[N-] ; CC(C)(C)OC(=O)N[C@@H]1C(=O)OCC1(C)C]

To a solution of 2 (2 g, 1 equiv.) in ethyl acetate (70 mL). Boc₂O (5.6 g, 2 equiv.) and Pd(OH)₂/C (0.2 g) were added into the flask. Hydrogenation with H₂ balloon for 24 h. Rf₂=0.71, Rf₃=0.43 Dichloromethane. Pd(OH)₂/C was filtered by Cilite. Concentrated in vacuum and purified by silica gel flash chromatography to afford 2.5 g white powder 3 (84.6%). UPLC-MS: calculated [M+Na]⁺: 252.12, found: 252.13.

¹H-NMR (400 MHz, CDCl₃) δ: 4.95 (d, 1H), 4.32 (d, 1H), 3.96 (s, 2H), 1.40 (s, 9H), 1.18 (s, 3H), 0.94 (s, 3H).

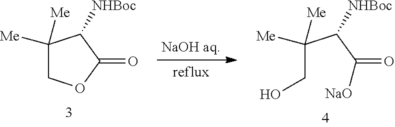

To a solution of 3 (150 mg, 1 equiv.) in NaOH aq. (5.2 mL, C_{NaOH}=0.125 mol/L, 1 equiv). The mixture was refluxed overnight. Once the reaction was detected be done by HPLC-MS, remove the water by freeze drying in vacuum to afford 4, which was used in subsequent step without further purification. UPLC-MS: calculated [M+H]⁺: 248.15, found: 247.99.

¹H-NMR (400 MHz, D₂O) δ: 3.49-3.39 (m, 2H), 1.46 (s, 9H), 0.98 (s, 3H), 0.94 (s, 3H).

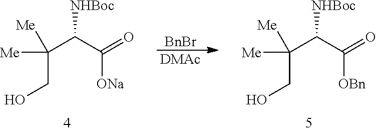

To a solution of previous product 3 (1 equiv.) in DMAc (2 mL). BnBr (115 μL, 1 equiv.) was added. The mixture was stirred for 12 h. Then remove the DMAc and purified by silica gel flash chromatography to afford 200 mg 5 (90.1% in two steps). Rf₅=0.13, hexane/ethyl acetate=8:2. UPLC-MS: calculated [M+H]⁺: 338.20, found: 338.32.

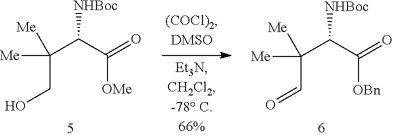 [SMILES: COC(=O)[C@@H](NC(=O)OC(C)(C)C)C(C)(C)CO ; CC(C)(C)OC(=O)N[C@H](C(=O)OCc1ccccc1)C(C)(C)C=O]

To a solution of oxalyl chloride (96 μL, 2.0 equiv.) in CH₂Cl₂ (5 mL), the mixture was stirred at −78° C. DMSO (80 μL, 2.0 equiv.) was added into the flask. The mixture was stirred for 10 min at −78° C. Then 6 (190 mg, 1 equiv.) in CH₂Cl₂ (1 mL) was added and stirred for 30 min. Et₃N (0.4 mL, 5.0 equiv.) was added and stirred for 30 min. The reaction was quenched by water. The organic phase was separated, washed with brine, dried, concentrated and the residue was purified by silica column chromatography to afford 6 125 mg (66%). Rf₆=0.3, hexane/ethyl acetate=8:2. UPLC-MS: calculated [M+H]⁺: 336.18, found: 336.29.

¹H-NMR. (CDCl₃, 100 MHz)

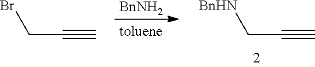

To a solution of benzyl amine (4.26 mg, 6 equiv.) in toluene (20 mL). Propargyl bromide (0.7 mL, 9.2M in toluene) was added. The reaction was stirred at room temperature overnight. The solvent was removed by vacuum and purified by silica column chromatography to afford 3. Rf₃=0.46, CH₂Cl₂/ethyl acetate=10:3. UPLC-MS: calculated [M+H]⁺: 146.10, found: 146

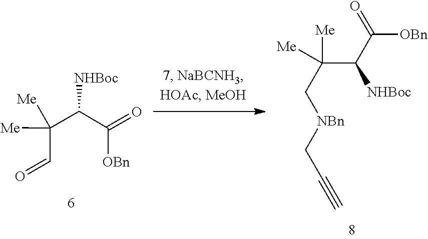 [SMILES: CC(C)(C)OC(=O)N[C@H](C(=O)OCc1ccccc1)C(C)(C)C=O ; C#CCN(Cc1ccccc1)CC(C)(C)[C@H](NC(=O)OC(C)(C)C)C(=O)OCc1ccccc1]

To a solution of 6 (1 equiv.) in DCE. 7 (2 equiv.) and HOAc (2 equiv.) were added. Then NaB(AcO)₃H (2 equiv.) was added into 8 portions. The mixture was stirred for 2 h. The solvent was removed by vacuum and purified by silica column chromatography to afford 8. UPLC-MS: calculated [M+H]⁺: 465.28, found: 465.30.

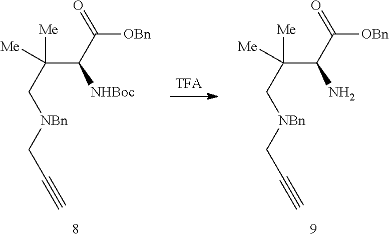 [SMILES: C#CCN(Cc1ccccc1)CC(C)(C)[C@H](NC(=O)OC(C)(C)C)C(=O)OCc1ccccc1 ; C#CCN(Cc1ccccc1)CC(C)(C)[C@H](N)C(=O)OCc1ccccc1]

To a solution of 8 (3 mg, 1 equiv.) in TFA (1 mL). The reaction was stirred for 5 min. Then the solvent was removed by vacuum to afford 8 without further purification. UPLC-MS: calculated [M+H]⁺: 365.22, found: 365.36.

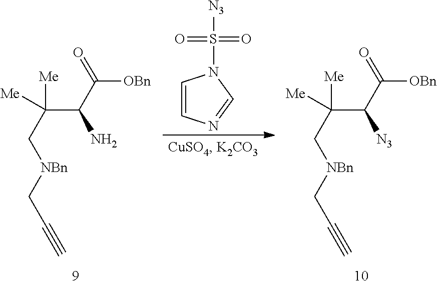 [SMILES: C#CCN(Cc1ccccc1)CC(C)(C)[C@H](N)C(=O)OCc1ccccc1 ; [N-]=[N+]=NS(=O)(=O)n1ccnc1 ; C#CCN(Cc1ccccc1)CC(C)(C)[C@H](N=[N+]=[N-])C(=O)OCc1ccccc1]

To a solution of 9 (3 mg, 1 equiv.) in MeOH (1 mL). CuSO4 (1 mg), Imidazole-1-sulfonyl azide (4 mg) and K₂CO₃ (6 mg) were added. The mixture was stirred for 2 h. Then the solvent was removed by vacuum and purified by silica column chromatography to afford 10. UPLC-MS: calculated [M+H]⁺: 391.21, found: 391.31. Retention time: 2.7 min

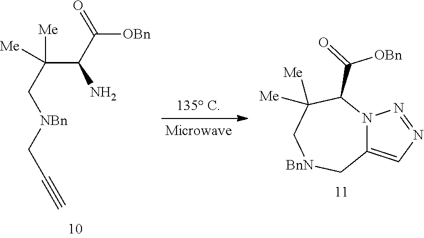 [SMILES: C#CCN(Cc1ccccc1)CC(C)(C)[C@H](N)C(=O)OCc1ccccc1 ; CC1(C)CN(Cc2ccccc2)Cc2cnnn2[C@@H]1C(=O)OCc1ccccc1]

To a solution of 10 (1 equiv.) in 1,2-DCB (1 mL). Microwave the reaction at 135° C. for 12 h. Then purify by silica column chromatography to afford 11. Rf₁₁=0.14, Hexane/ethyl acetate=7:3. UPLC-MS: calculated [M+H]⁺: 391.21, found: 391.25. Retention time: 2.2 min

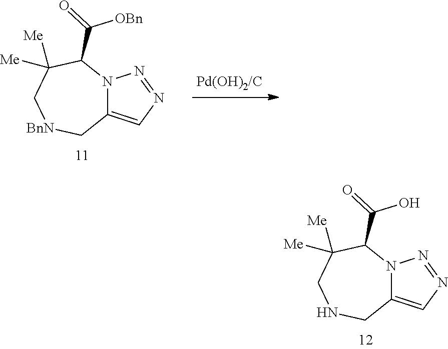 [SMILES: CC1(C)CN(Cc2ccccc2)Cc2cnnn2[C@@H]1C(=O)OCc1ccccc1 ; CC1(C)CNCc2cnnn2[C@@H]1C(=O)O]

To a solution of 11 (1 equiv.) in ethyl acetate (1 mL). Pd(OH)₂/C (10% wt) was added. The mixture was stirred under H₂ atmosphere overnight. The mixture was filtered by Celite and concentrated in vacuum to afford 12. UPLC-MS: calculated [M+H]⁺: 211.12, found: 211.09.

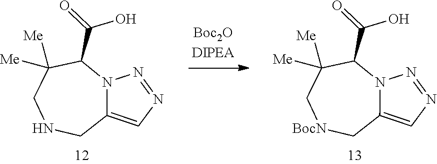 [SMILES: CC1(C)CNCc2cnnn2[C@@H]1C(=O)O ; CC(C)(C)OC(=O)N1Cc2cnnn2[C@H](C(=O)O)C(C)(C)C1]

To a solution of 12 (1 equiv.) in ethyl acetate (1 mL). DIPEA (5 equiv.) and Boc₂O (1.5 equiv.) were added. The mixture was stirred for 30 min. Then Purified by silica column chromatography to afford 13. UPLC-MS: calculated [M+H]⁺: 311.17, found: 311.09.

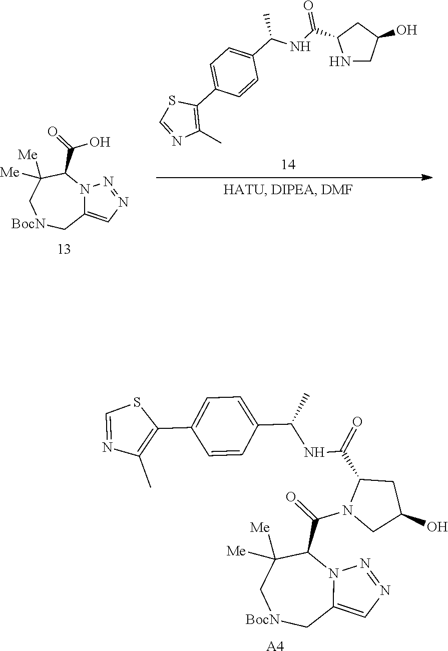

To a solution of 13 (1 equiv.) in DMF. Then DIPEA (5 equiv.) and HATU (1 equiv.) were added. The mixture was stirred for 10 min. Then 14 (1.5 equiv.) was added and stirred for another 10 min. The mixture was detected by UPLC-MS and purified by HPLC to afford A4. UPLC-MS: calculated [M+H]⁺: 624.30, found: 624.19.

### Compound A5. (2S,4R)-4-hydroxy-N-((S)-1-(4-(4-methylthiazol-5-yl)phenyl)ethyl)-1-((S)-5,6,7,8-tetrahydro-4H-[1,2,3]triazolo[1,5-a]azepine-8-carbonyl)pyrrolidine-2-carboxamide

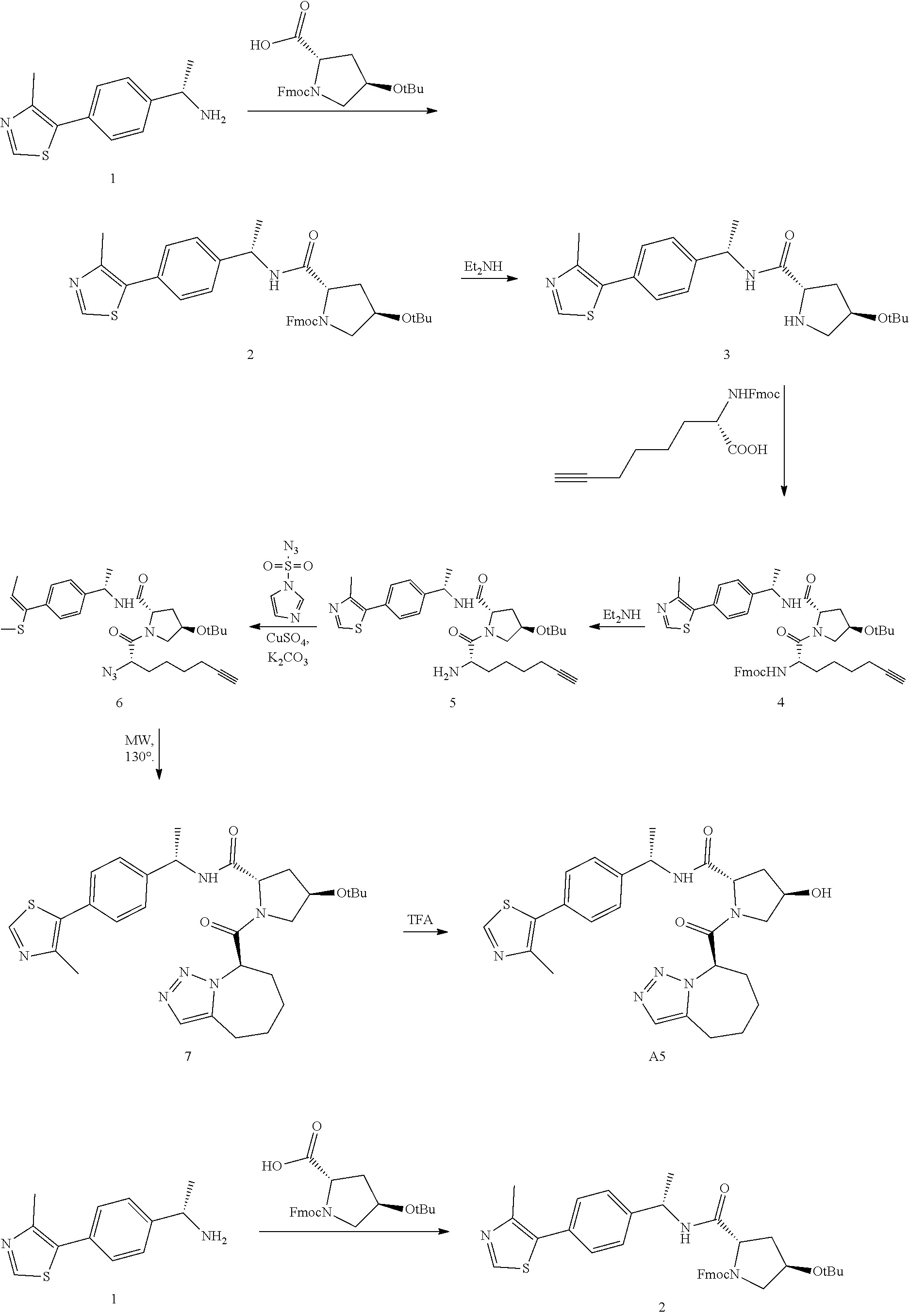

To a solution of 1 (2.55 g, 1 equiv.) and (2S,4R)-1-(((9H-fluoren-9-yl)methoxy)carbonyl)-4-(tert-butoxy)pyrrolidine-2-carboxylic acid (4.1 g, 1 equiv.) in DMF. Then DIPEA (9 mL. 5 equiv.) and HATU (3.8 g 1 equiv.) were added. The mixture was stirred for 10 min. The mixture was detected by UPLC-MS and purified by HPLC to afford 2. UPLC-MS: calculated [M+H]+:610.3, found: 610.5.

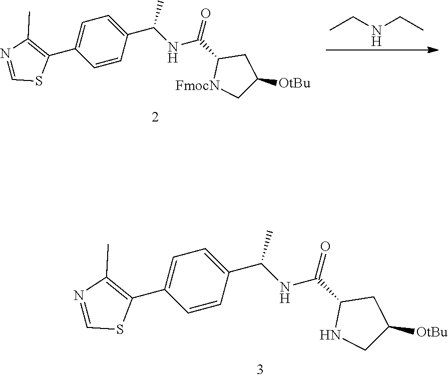

To a solution of 2 (1 g, 1 equiv.) MeCN. Then diethylamine (0.85 mL. 5 equiv.) was added. The mixture was stirred for 3 hours. The mixture was detected by UPLC-MS and purified by HPLC to afford 3. UPLC-MS: calculated [M+H]+:388.2, found: 388.1.

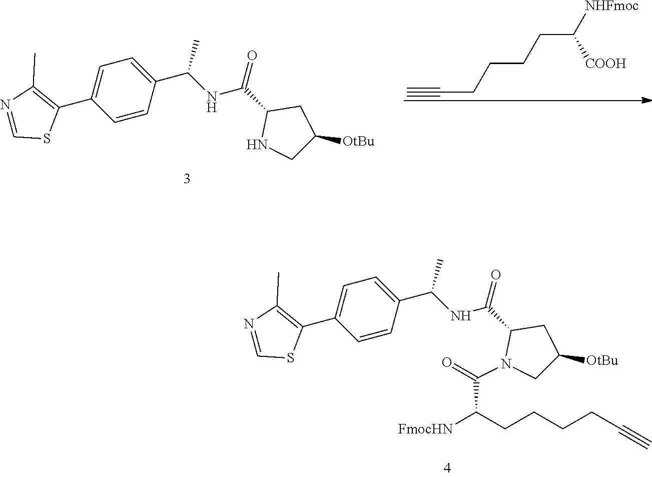

To a solution of (S)-2-((((9H-fluoren-9-yl)methoxy)carbonyl)amino)oct-7-ynoic acid (188 mg, 1 equiv.) and 3 (193 mg, 1 equiv.) DMF. Then DIPEA (0.44 mL. 5 equiv.) and HATU (190 mg 1 equiv.) were added. The mixture was stirred for 10 min. The mixture was detected by UPLC-MS and purified by HPLC to afford 4. UPLC-MS: calculated [M+H]+:747.4, found: 747.5.

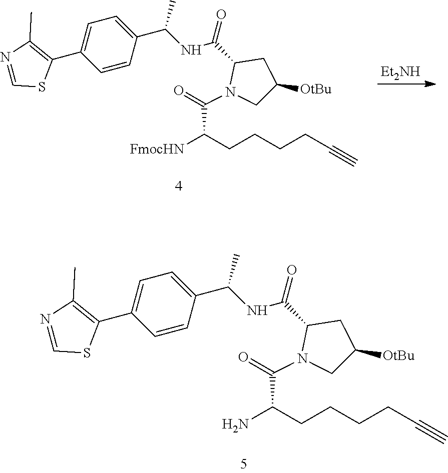

To a solution of 5 (250 mg, 1 equiv.) MeCN. Then diethylamine (173 μL. 5 equiv.) was added. The mixture was stirred for 3 hours. The mixture was detected by UPLC-MS and purified by HPLC to afford 5. UPLC-MS: calculated [M+H]+:525.3, found: 525.6.

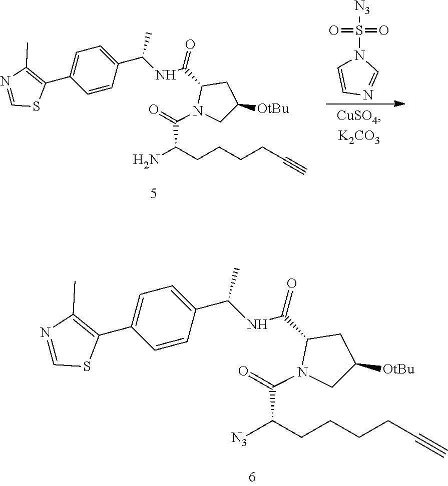 [SMILES: C#CCCCC[C@H](N)C(=O)N1C[C@H](OC(C)(C)C)C[C@H]1C(=O)N[C@@H](C)c1ccc(-c2scnc2C)cc1 ; [N-]=[N+]=NS(=O)(=O)n1ccnc1 ; C#CCCCC[C@H](N=[N+]=[N-])C(=O)N1C[C@H](OC(C)(C)C)C[C@H]1C(=O)N[C@@H](C)c1ccc(-c2scnc2C)cc1]

1H-imidazole-1-sulfonyl azide (72 mg, 2 equiv.) was added to a mixture of 5 (0.1 g, 1 equiv.), K2CO3 (109 mg, 5 equiv.) and CuSO4 (5.4 mg, 0.2 equiv.) in MeOH at 25° C. The reaction mixture was stirred at 25° C. for 10 h. After removing the solvent, The organic layer was concentrated, and the residue was purified by reverse phase preparative HPLC to give 6. UPLC-MS calculated [M+H]+: 551.3, found: 551.3.

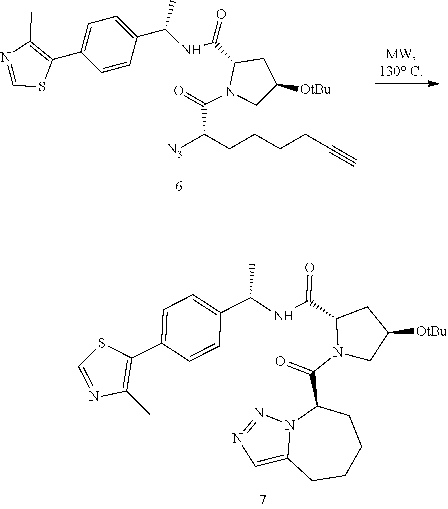 [SMILES: C#CCCCC[C@H](N=[N+]=[N-])C(=O)N1C[C@H](OC(C)(C)C)C[C@H]1C(=O)N[C@@H](C)c1ccc(-c2scnc2C)cc1 ; Cc1ncsc1-c1ccc([C@H](C)NC(=O)[C@@H]2C[C@@H](OC(C)(C)C)CN2C(=O)[C@H]2CCCCc3cnnn32)cc1]

To a solution of 6 (1 equiv.) in 1,2-DCB (1 mL). Microwave the reaction at 130° C. for 12 h. Then purify by silica column chromatography to afford 7. UPLC-MS: calculated [M+H]+: 551.3, found: 551.4.

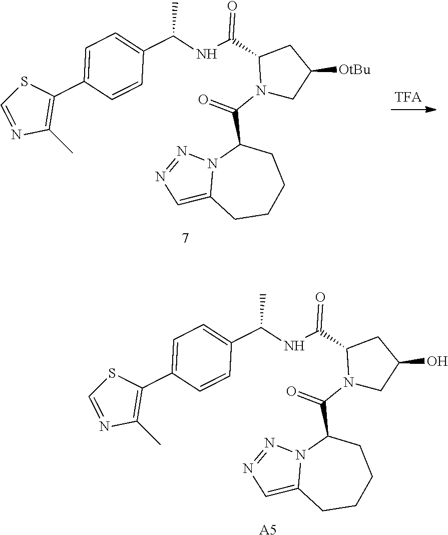

To a solution of 7 (1 equiv.) in CH₂Cl₂ (1 mL). TFA (0.5 mL) was added. The reaction was stirred for 1 hour. After removing the solvent, then purify by HPLC to afford A5. UPLC-MS: calculated [M+H]⁺: 495.2, found: 495.3.

### Compound A6. (2S,4R)-1-((S)-7,7-dimethyl-5,6,7,8-tetrahydro-4H-[1,2,3]triazolo[1,5-a][1,4]diazepine-8-carbonyl)-4-hydroxy-N-((S)-1-(4-(4-methylthiazol-5-yl)phenyl)ethyl)pyrrolidine-2-carboxamide

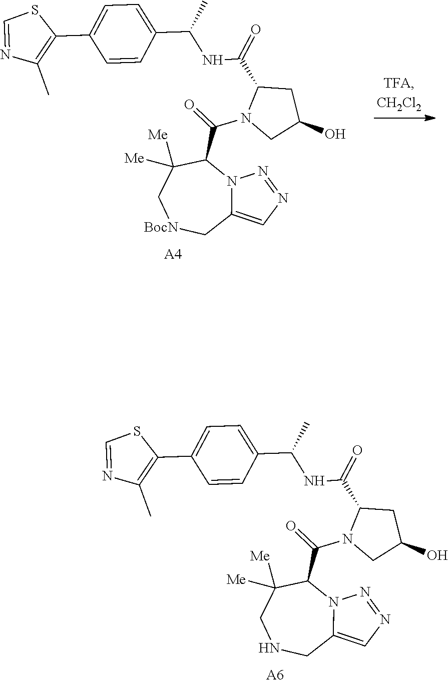

To a solution of A4 (1 equiv.) in CH₂Cl₂ (c_{A4}=0.05 mol/L), TFA (half volume of CH₂Cl₂) was added. The mixture was stirred for 30 min and detected by UPLC-MS and purified by HPLC to afford A6. UPLC-MS: calculated [M+H]⁺: 524.2, found: 524.6

### Compound A7. (2S,4R)-4-hydroxy-N—((S)-1-(4-(4-methylthiazol-5-yl)phenyl)ethyl)-1-((S)-5,7,7-trimethyl-5,6,7,8-tetrahydro-4H-[1,2,3]triazolo[1,5-a][1,4]diazepine-8-carbonyl)pyrrolidine-2-carboxamide

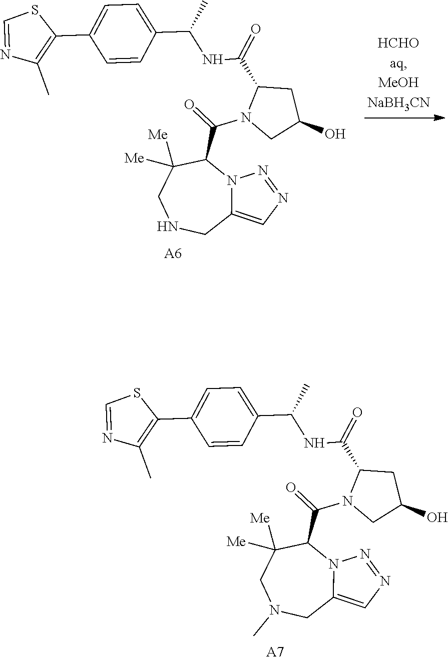

To a solution of A6 (1 equiv.) in MeOH (c_{A6}=0.1 mol/L), HCHO aq. (37% formaldehyde solution, 13.4 mol/L, 5 equiv) was added. The mixture was stirred for 30 min. Then NaBH₃CN (3 equiv.) was added in 5 portions. The mixture was detected by UPLC-MS and purified by HPLC to afford A7. UPLC-MS: calculated [M+H]⁺: 538.3, found: 538.4

### Compound A8. (2S,4R)-1-((S)-5-acetyl-7,7-dimethyl-5,6,7,8-tetrahydro-4H-[1,2,3]triazolo[1,5-a][1,4]diazepine-8-carbonyl)-4-hydroxy-N—((S)-1-(4-(4-methylthiazol-5-yl)phenyl)ethyl)pyrrolidine-2-carboxamide

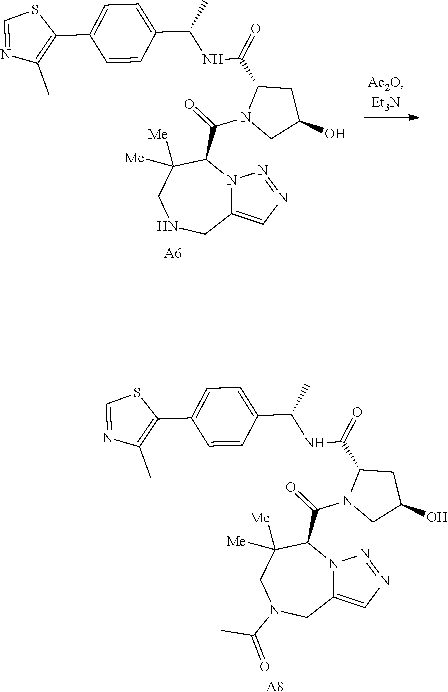

To a solution of A6 (1 equiv.) in CH₂Cl₂ (c_{A4}=0.05 mol/L). Et₃N (5 equiv.) was added at room temperature. The mixture was stirred for 1 min, then Ac₂O (1.2 equiv.) was added. The reaction was detected by UPLC-MS. Solvent was removed in vacuum and purified by HPLC to afford A8. UPLC-MS: calculated [M+H]⁺: 566.3, found: 566.8.

### Compound A9. (2S,4R)-1-((S)-5-benzyl-3-cyclopropyl-7,7-dimethyl-5,6,7,8-tetrahydro-4H-[1,2,3]triazolo[1,5-a][1,4]diazepine-8-carbonyl)-4-hydroxy-N—((S)-1-(4-(4-methylthiazol-5-yl)phenyl)ethyl)pyrrolidine-2-carboxamide

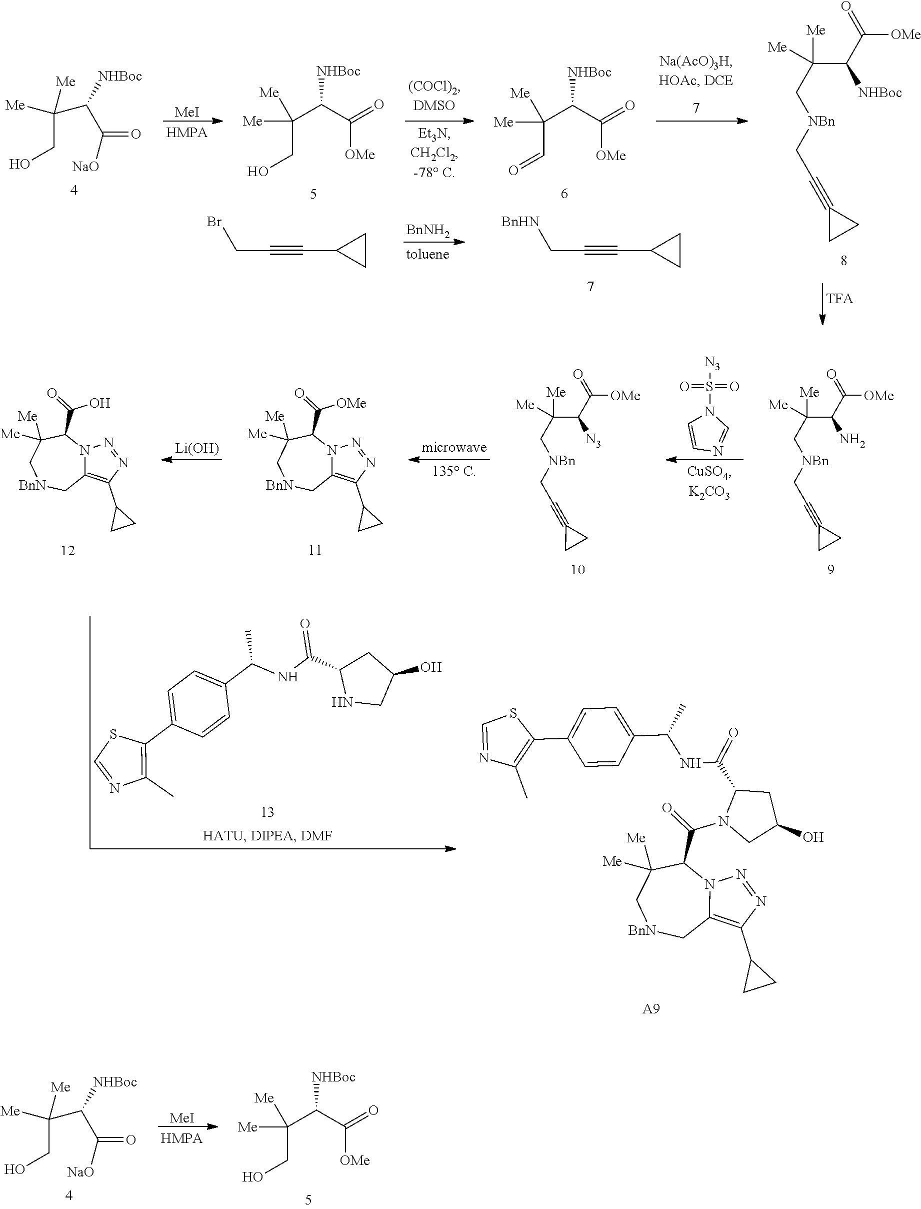

To a solution of previous product 4 (1 equiv.) in HMPA (c₄=0.5 mol/L). Mel (3 equiv.) was added. The mixture was stirred for 12 h. Then purified by silica gel flash chromatography to afford 5. UPLC-MS: calculated [M+H]⁺: 262.2, found: 262.8.

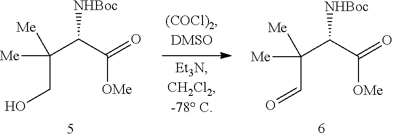 [SMILES: COC(=O)[C@@H](NC(=O)OC(C)(C)C)C(C)(C)CO ; COC(=O)[C@@H](NC(=O)OC(C)(C)C)C(C)(C)C=O]

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 5 (1 equiv.) in CH₂Cl₂ was added and stirred for 30 min. Et₃N (5.0 equiv.) was added and stirred for 30 min. The reaction was quenched by water. The organic phase was separated, washed with brine, dried, concentrated and the residue was purified by silica column chromatography to afford 6 UPLC-MS: calculated [M+H]⁺: 260.2, found: 260.2.

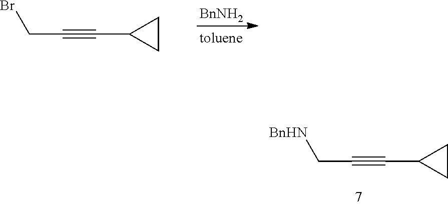

To a solution of benzyl amine (6 equiv.) in toluene (20 mL). (3-bromoprop-1-yn-1-yl)cyclopropane (1 equiv.) was added. The reaction was stirred at room temperature overnight. The solvent was removed by vacuum and purified by silica column chromatography to afford 7. UPLC-MS: calculated [M+H]⁺: 186.1, found: 186.2

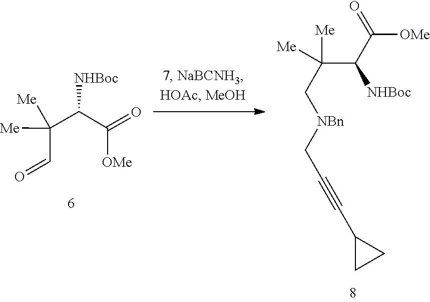 [SMILES: COC(=O)[C@@H](NC(=O)OC(C)(C)C)C(C)(C)C=O ; COC(=O)[C@@H](NC(=O)OC(C)(C)C)C(C)(C)CN(CC#CC1CC1)Cc1ccccc1]

To a solution of 6 (1 equiv.) in DCE. 7 (2 equiv.) and HOAc (2 equiv.) were added. Then NaB(AcO)₃H (2 equiv.) was added into 8 portions. The mixture was stirred for 2 h. The solvent was removed by vacuum and purified by silica column chromatography to afford 8. UPLC-MS: calculated [M+H]⁺: 429.3, found: 429.5.

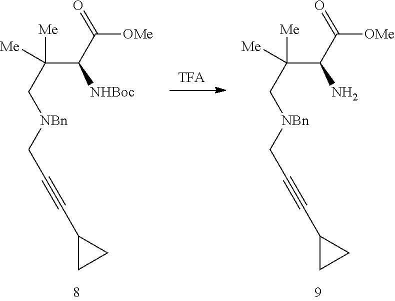 [SMILES: COC(=O)[C@@H](NC(=O)OC(C)(C)C)C(C)(C)CN(CC#CC1CC1)Cc1ccccc1 ; COC(=O)[C@@H](N)C(C)(C)CN(CC#CC1CC1)Cc1ccccc1]

To a solution of 8 (1 mmol) in CH₂Cl₂ (2 mL), TFA (1 mL) was added. The mixture was stirred for 30 min and detected by UPLC-MS and removed solvent in vacuum to afford 9 without further purification. UPLC-MS: calculated [M+H]⁺: 329.2, found: 329.3.

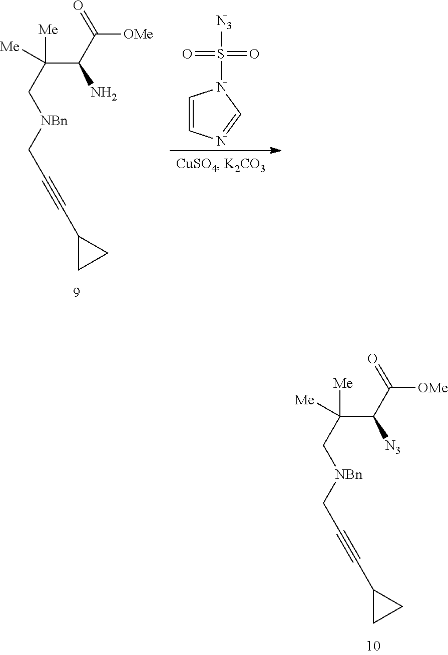 [SMILES: COC(=O)[C@@H](N)C(C)(C)CN(CC#CC1CC1)Cc1ccccc1 ; [N-]=[N+]=NS(=O)(=O)n1ccnc1 ; COC(=O)[C@@H](N=[N+]=[N-])C(C)(C)CN(CC#CC1CC1)Cc1ccccc1]

To a solution of 9 (1 equiv.) in MeOH. CuSO4 (0.2 equiv.), Imidazole-1-sulfonyl azide (2 equiv) and K₂CO₃ (3 equiv.) were added. The mixture was stirred for 2 h. Then the solvent was removed by vacuum and purified by silica column chromatography to afford 10. UPLC-MS: calculated [M+H]⁺: 355.2, found: 355.0.

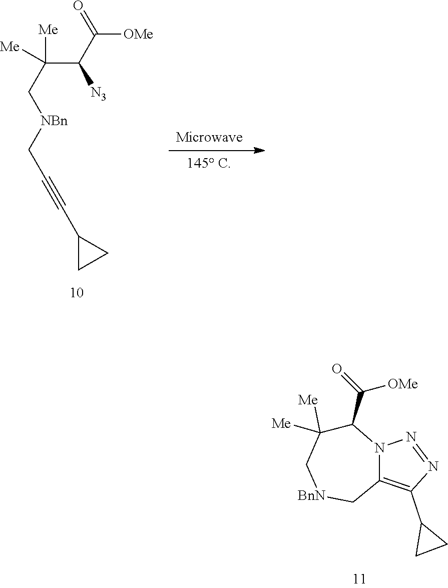

To a solution of 10 (1 equiv.) in 1,2-DCB (c₁₀=0.05 mol/L). Microwave the reaction at 145° C. for 16 h. Then purify by silica column chromatography to afford 11. UPLC-MS: calculated [M+H]⁺: 355.2, found: 355.2.

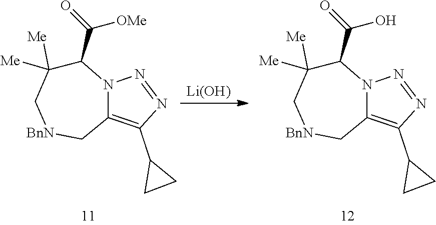

To a solution of 11 (1 equiv.) in MeOH/H₂O (c₁₁=0.1 mol/L, MeOH:H₂O=3:1). LiOH (3 equiv.) was added at room temperature. The mixture was stirred overnight, and purified by HPLC to afford 12. UPLC-MS: calculated [M+H]⁺: 341.2, found: 341.7.

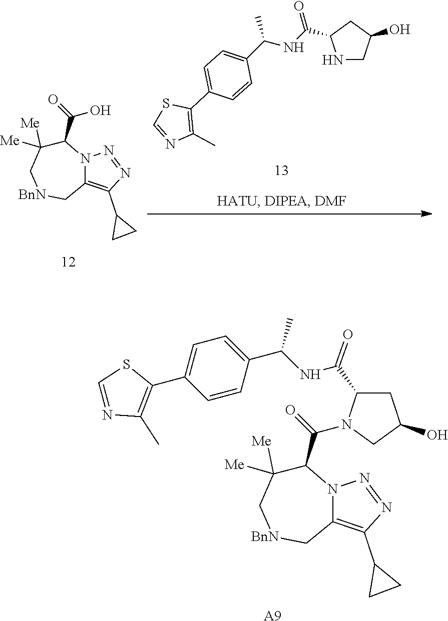

To a solution of 12 (1 equiv.) in DMF. Then DIPEA (5 equiv.) and HATU (1 equiv.) were added. The mixture was stirred for 10 min. Then 13 (1.5 equiv.) was added and stirred for another 10 min. The mixture was detected by UPLC-MS and purified by HPLC to afford A9. UPLC-MS: calculated [M+H]+:654.3, found: 654.1.

### Compound A10. (2S,4R)-1-((S)-3-cyclopropyl-7,7-dimethyl-5,6,7,8-tetrahydro-4H-[1,2,3]triazolo[1,5-a][1,4]diazepine-8-carbonyl)-4-hydroxy-N—((S)-1-(4-(4-methylthiazol-5-yl)phenyl)ethyl)pyrrolidine-2-carboxamide

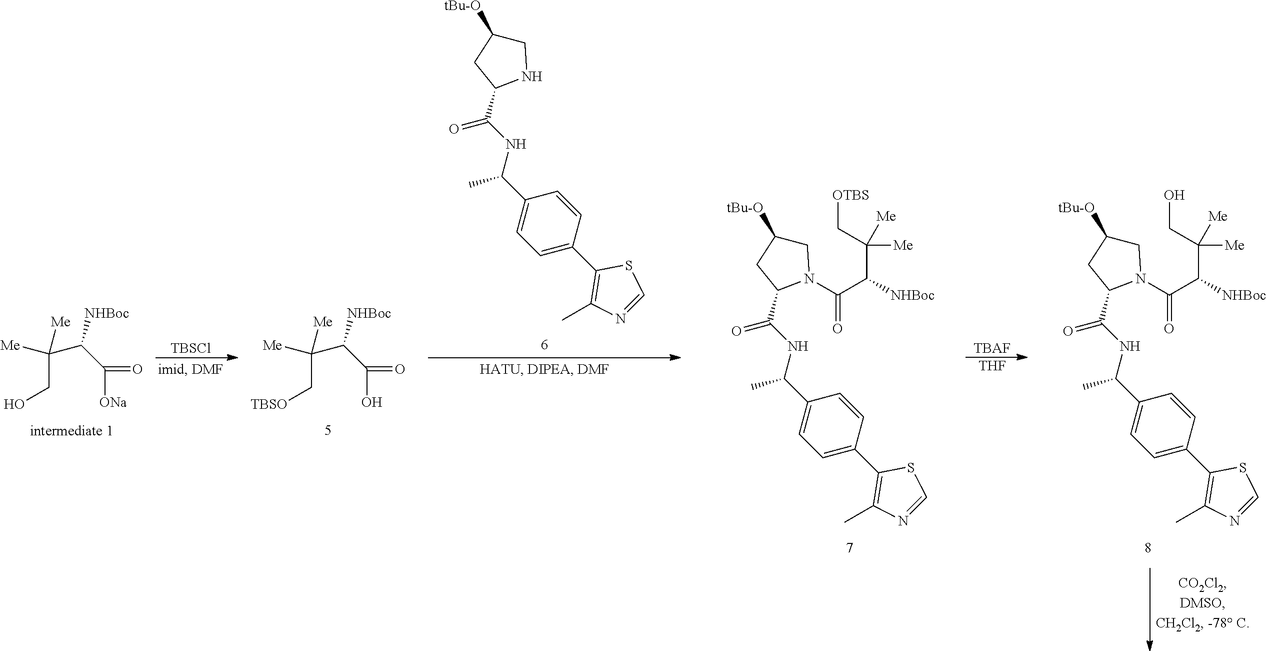

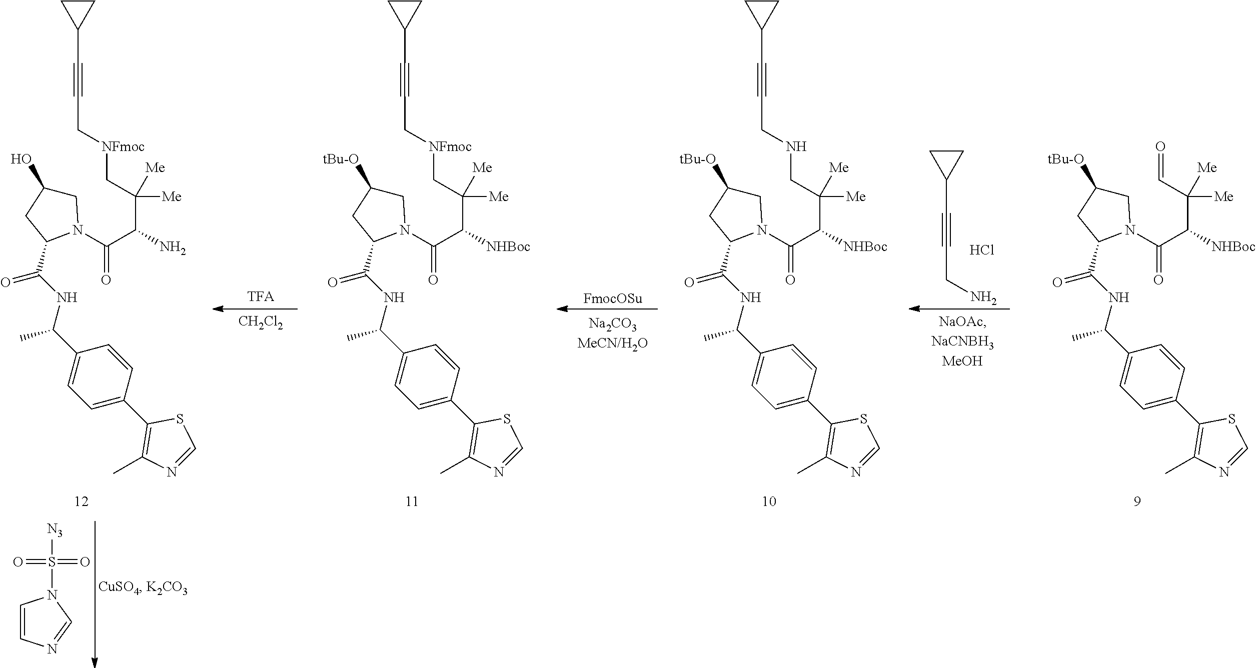

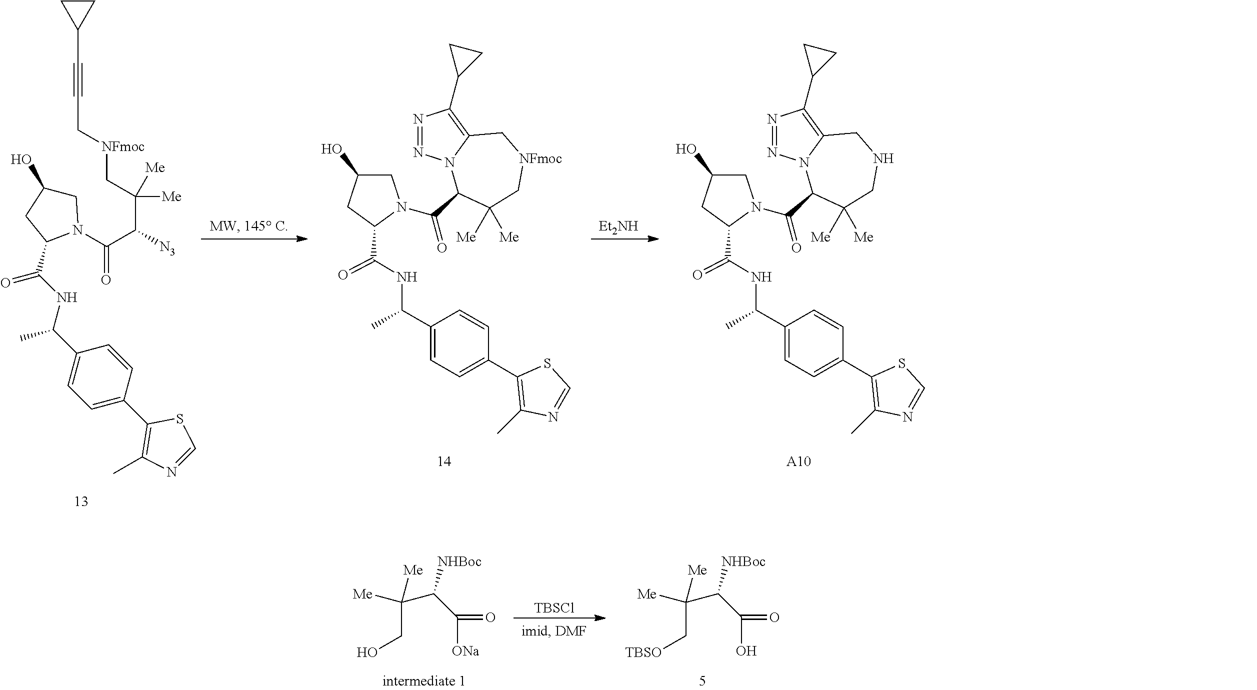

To a solution of intermediate 1 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

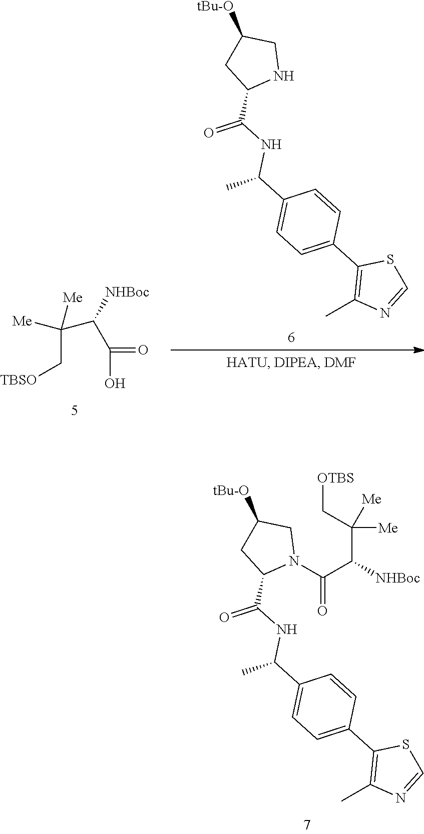

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 731.4, found: 731.6.

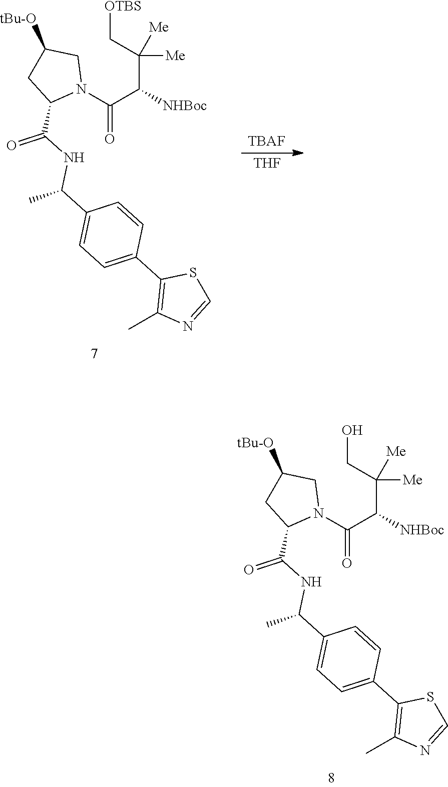

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+H]⁺: 617.3, found: 617.6.

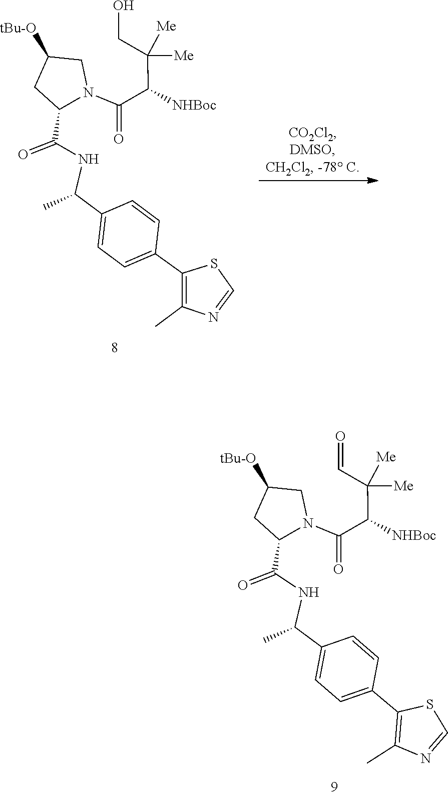

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 615.3, found: 615.7.

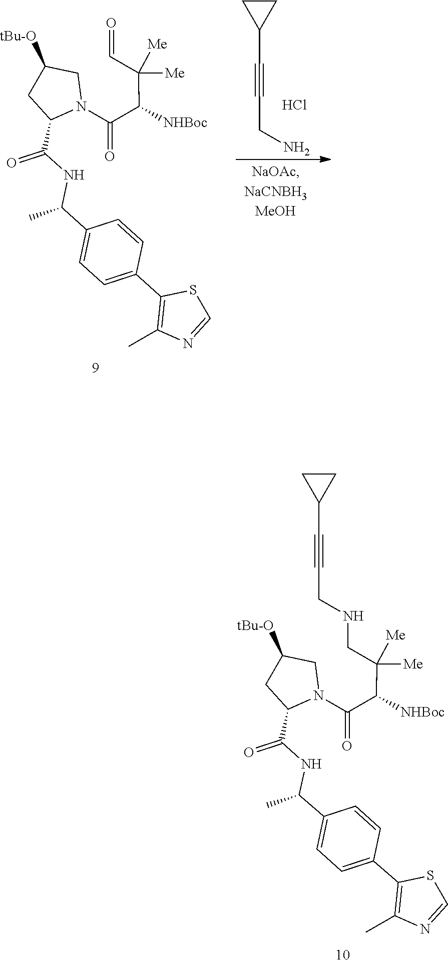

To a solution of 9 (1 equiv.) in MeOH. 3-cyclopropylprop-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 694.4, found: 694.5.

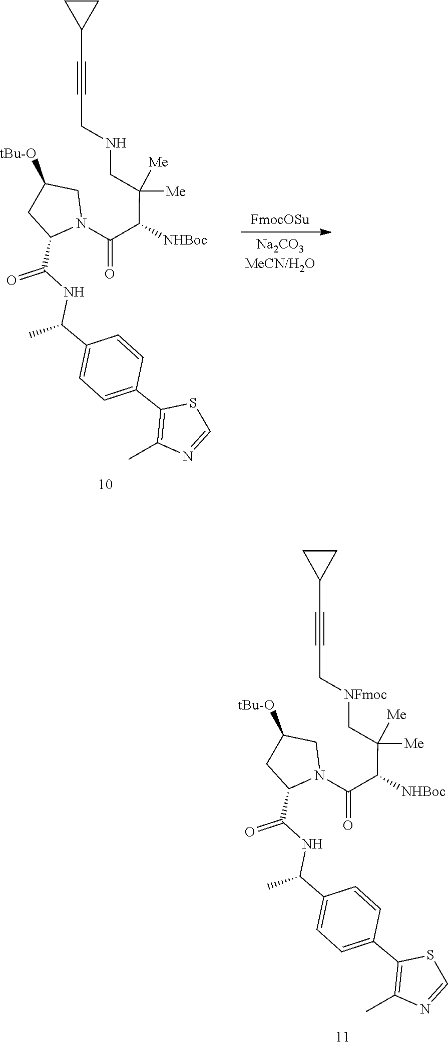

To a solution of 10 (1 equiv.) in MeCN/H₂O (2:1). Na₂CO₃ (3 equiv.) and FmocOSu (2.0 equiv.) were added. The reaction was stirred for 6 hours. The ethyl acetate was added and water phase was washed twice by ethyl acetate. The organic phase was combined, dried, concentrated by vacuum and purified by silica column chromatography to afford 10. UPLC-MS: calculated [M+H]⁺: 916.5, found: 916.8.

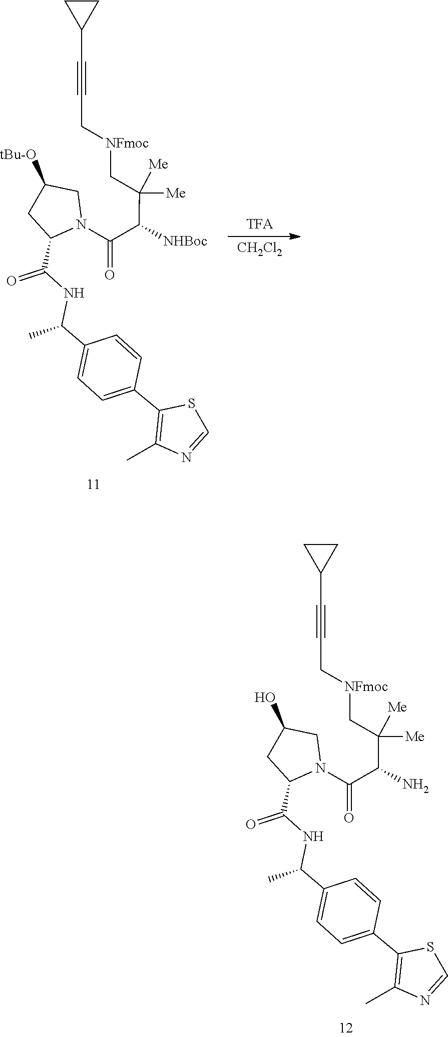

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 760.4, found: 760.3.

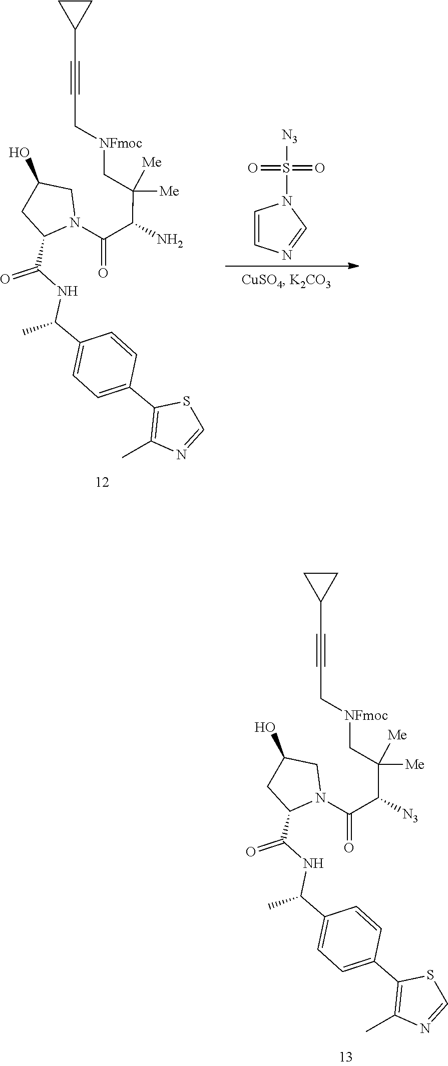

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 786.3, found: 786.5.

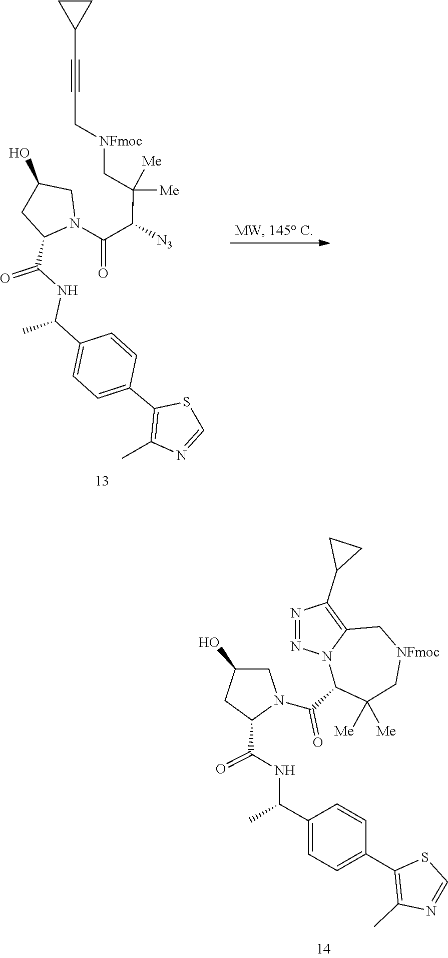

To a solution of 13 (1 equiv.) in 1,2-DCB (c₁₃=0.01 mol/L). Microwave the reaction at 145° C. for 16 h. The solvent was removed in vacuum for next step without further purification. UPLC-MS: calculated [M+H]⁺: 786.3, found: 786.4.

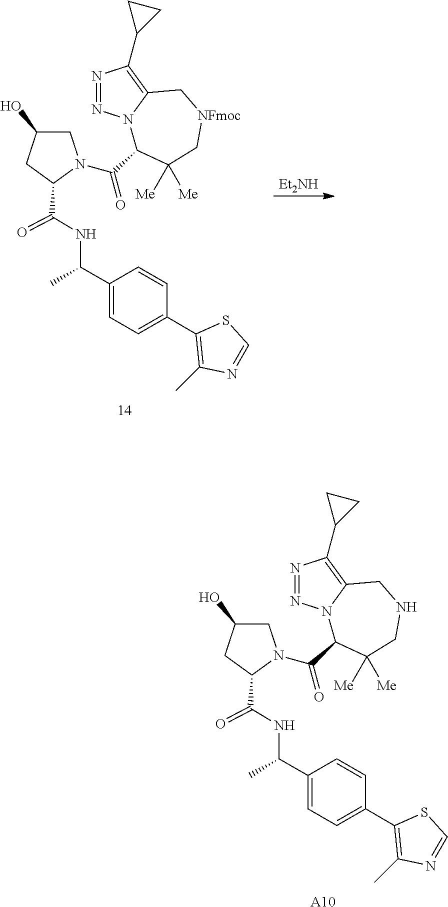

To a solution of 14 (1 equiv.) MeCN. Then diethylamine (5 equiv.) was added. The mixture was stirred for 3 hours. The mixture was detected by UPLC-MS and purified by HPLC to afford A10. UPLC-MS: calculated [M+H]⁺: 564.3, found: 564.3.

### Compound A11. methyl (S)-3-((2S,4R)-1-((S)-7,7-dimethyl-7,8-dihydro-4H-[1,2,3]triazolo[1,5-a]azepine-8-carbonyl)-4-hydroxypyrrolidine-2-carboxamido)-3-(4-(4-methylthiazol-5-yl)phenyl)propanoate

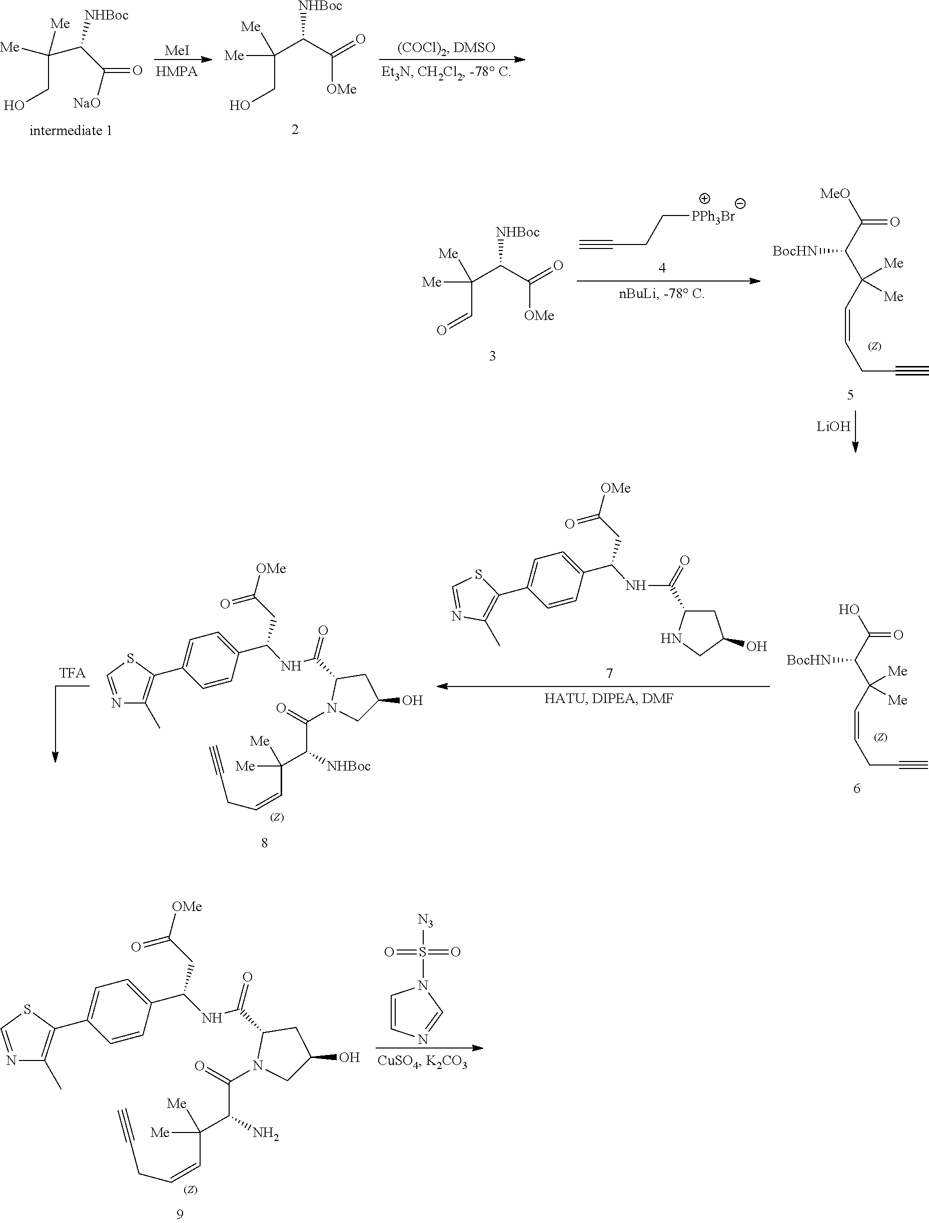

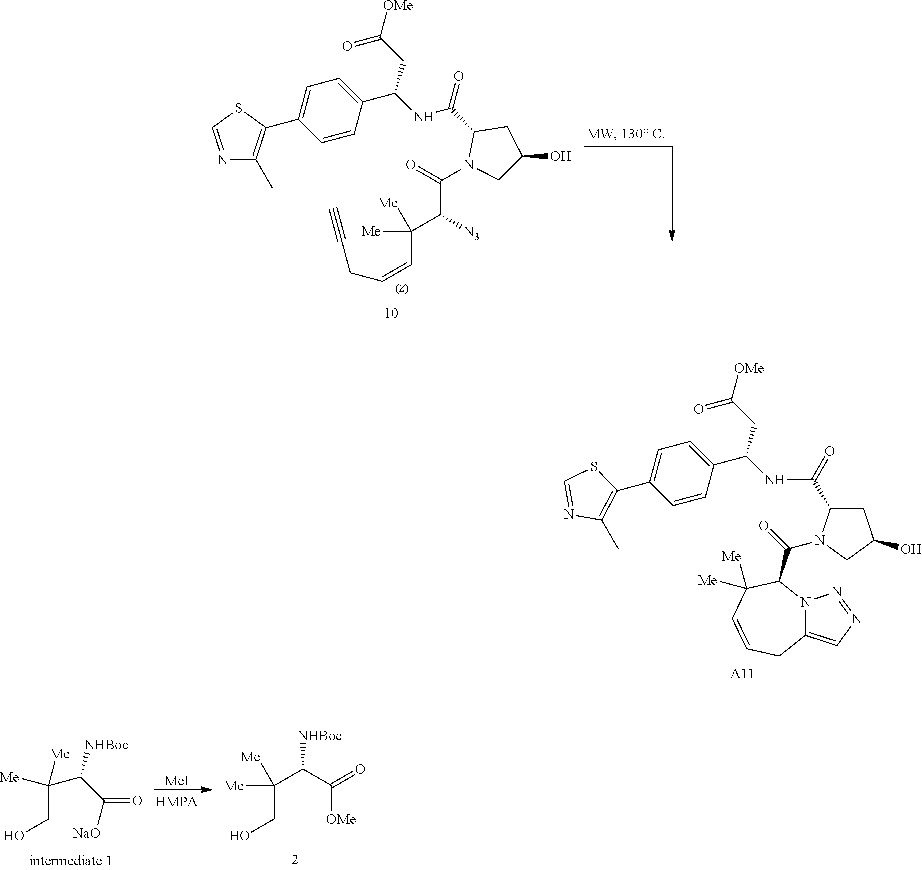

To a solution of 4 (1 equiv.) HMPA. Then Mel (2 equiv.) was added. The mixture was stirred for 3 hours and purified by silica column chromatography for afford 2. UPLC-MS: calculated [M+H]+:262.2, found: 262.3.

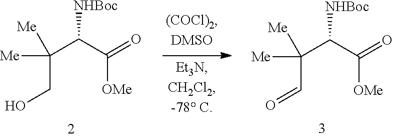 [SMILES: COC(=O)[C@@H](NC(=O)OC(C)(C)C)C(C)(C)CO ; COC(=O)[C@@H](NC(=O)OC(C)(C)C)C(C)(C)C=O]

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 2 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 3 UPLC-MS: calculated [M+H]⁺: 260.2, found: 260.1.

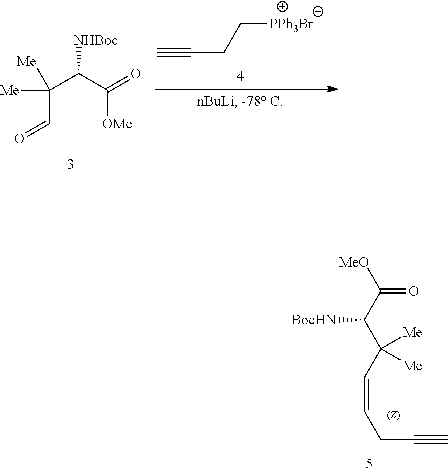

To a solution of 4 (1.2 equiv.) in THF, the mixture was stirred at −78° C. n-BuLi (1.6M in hexane, 1 equiv.) was added into flask. The mixture was stirred at −78° C. for 1 hour. 3 (1 equiv. in THF) was added into flask, the mixture was stirred for 30 min at −78° C., then warm to room temperature and stirred for more 2 hours. The reaction was quenched by saturated NaHCO₃ aq. Ethyl acetate was added, water phase was washed twice by ethyl acetate. Combined organic phase and dried by MgSO₄, concentrated and purified by silica column chromatography to afford 5 UPLC-MS: calculated [M+H]⁺: 296.2, found: 296.3.

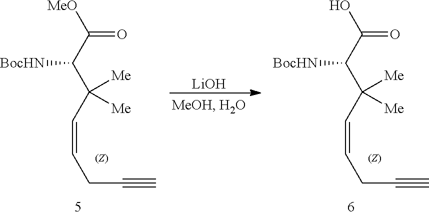 [SMILES: C#CC/C=C\C(C)(C)[C@H](NC(=O)OC(C)(C)C)C(=O)OC ; "LiOH" ; C#CC/C=C\C(C)(C)[C@H](NC(=O)OC(C)(C)C)C(=O)O]

To a solution of 5 (1 equiv.) in MeOH/H₂O (3:1), LiOH (2 equiv.) was added. The reaction was stirred for 6 hours. Then HCl (2M, 2 equiv.) was added, the solvent was removed in vacuum and purified by silica column chromatography to afford 6. UPLC-MS: calculated [M+H]⁺: 282.2, found: 282.5.

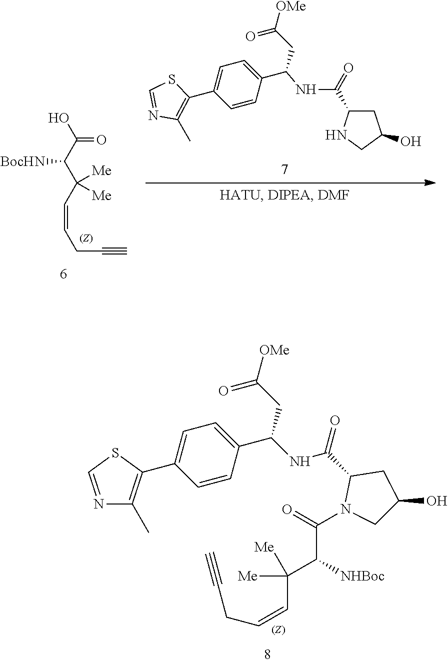 [SMILES: C#CC/C=C\C(C)(C)[C@H](NC(=O)OC(C)(C)C)C(=O)O ; COC(=O)C[C@H](NC(=O)[C@@H]1C[C@@H](O)CN1)c1ccc(-c2scnc2C)cc1 ; C#CC/C=C\C(C)(C)[C@@H](NC(=O)OC(C)(C)C)C(=O)N1C[C@H](O)C[C@H]1C(=O)N[C@@H](CC(=O)OC)c1ccc(-c2scnc2C)cc1]

To a solution of 6 (1 equiv.) and 7 (1 equiv.) in DMF. Then DIPEA (5 equiv.) and HATU (1 equiv.) were added. The mixture was stirred for 10 min. The mixture was detected by UPLC-MS and purified by HPLC to afford 8. UPLC-MS: calculated [M+H]⁺: 653.3, found: 653.4.

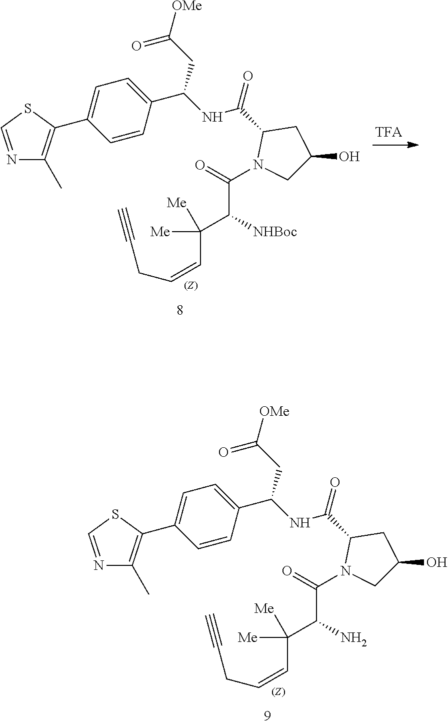 [SMILES: C#CC/C=C\C(C)(C)[C@@H](NC(=O)OC(C)(C)C)C(=O)N1C[C@H](O)C[C@H]1C(=O)N[C@@H](CC(=O)OC)c1ccc(-c2scnc2C)cc1 ; C#CC/C=C\C(C)(C)[C@@H](N)C(=O)N1C[C@H](O)C[C@H]1C(=O)N[C@@H](CC(=O)OC)c1ccc(-c2scnc2C)cc1]

8 was dissolved in CH₂Cl₂ (c₅=0.2 mol/L), TFA (1/2 volume of CH₂Cl₂) was added. The reaction was stirred for 1 hours. Solvent and TFA were removed in vacuum for next step without further purification. UPLC-MS: calculated [M+H]⁺: 553.3, found: 553.4.

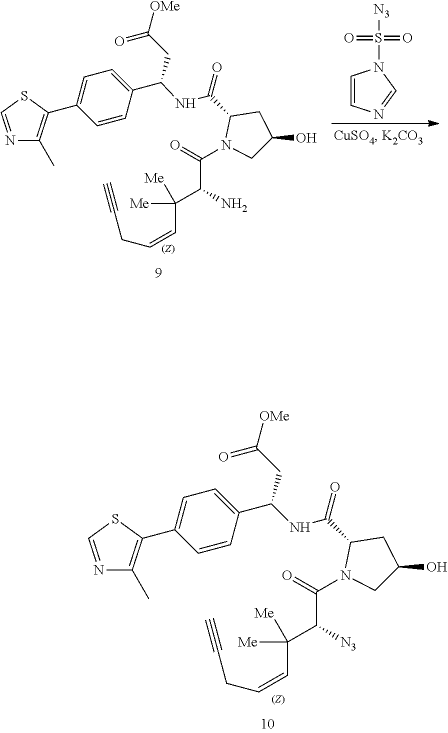 [SMILES: C#CC/C=C\C(C)(C)[C@@H](N)C(=O)N1C[C@H](O)C[C@H]1C(=O)N[C@@H](CC(=O)OC)c1ccc(-c2scnc2C)cc1 ; [N-]=[N+]=NS(=O)(=O)n1ccnc1 ; C#CC/C=C\C(C)(C)[C@@H](N=[N+]=[N-])C(=O)N1C[C@H](O)C[C@H]1C(=O)N[C@@H](CC(=O)OC)c1ccc(-c2scnc2C)cc1]

1H-imidazole-1-sulfonyl azide (2 equiv.) was added to a mixture of 9 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 10. UPLC-MS calculated [M+H]+: 579.2, found: 579.3.

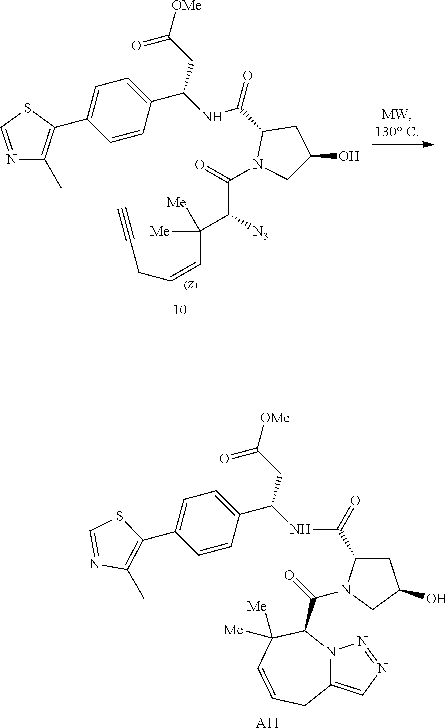

To a solution of 10 (1 equiv.) in 1,2-DCB (c₁₃=0.01 mol/L). Microwave the reaction at 135° C. for 12 h. The solvent was removed in vacuum for next step without further purification to afford A11. UPLC-MS: calculated [M+H]⁺: 579.2, found: 579.4.

### Compound A12. methyl (S)-3-((2S,4R)-1-((S)-5-acetyl-3-cyclopropyl-7,7-dimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanoate

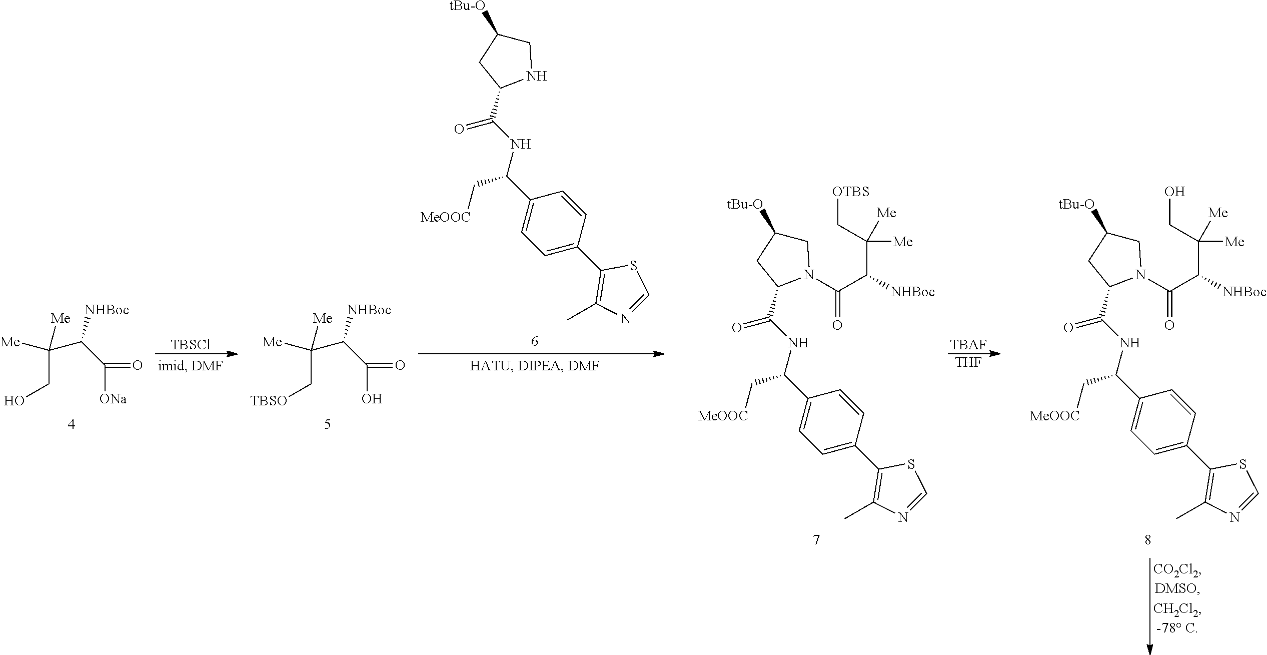

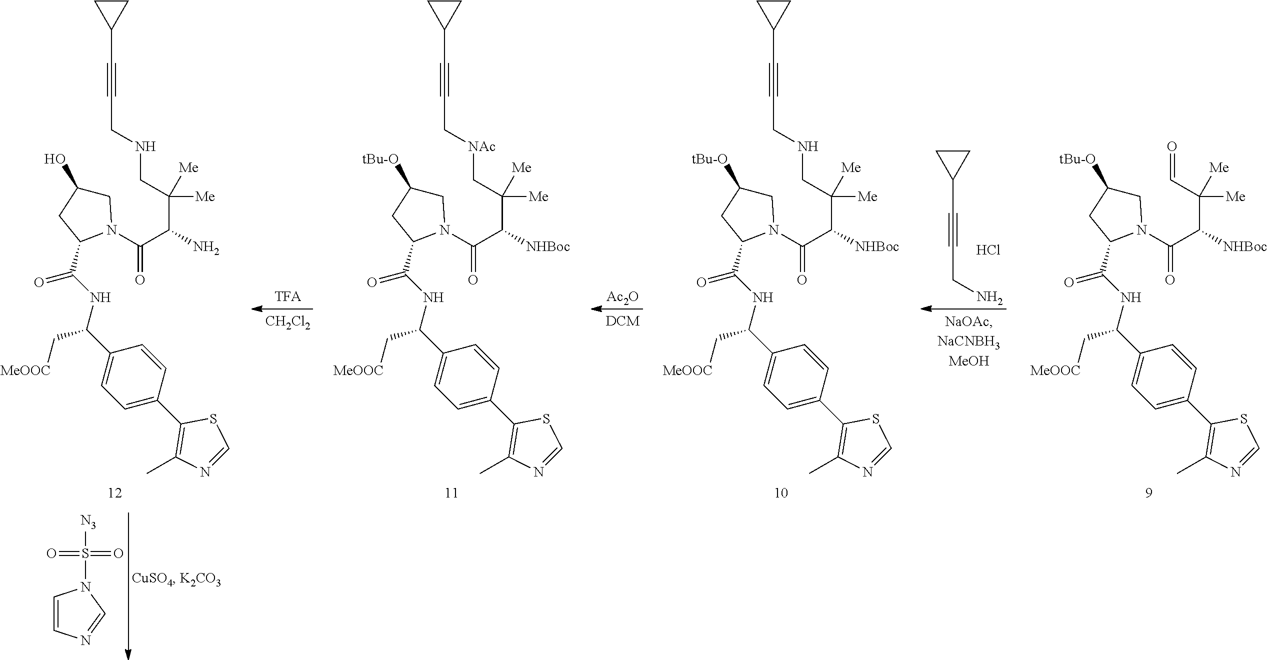

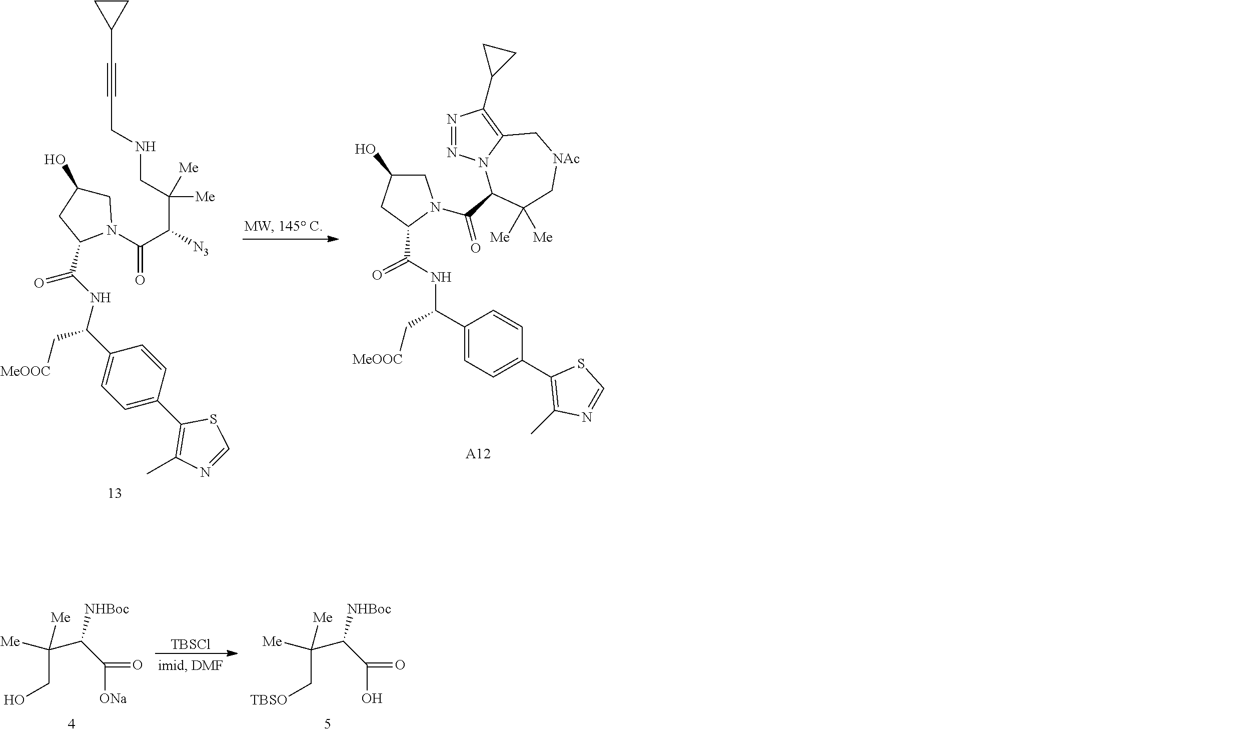

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

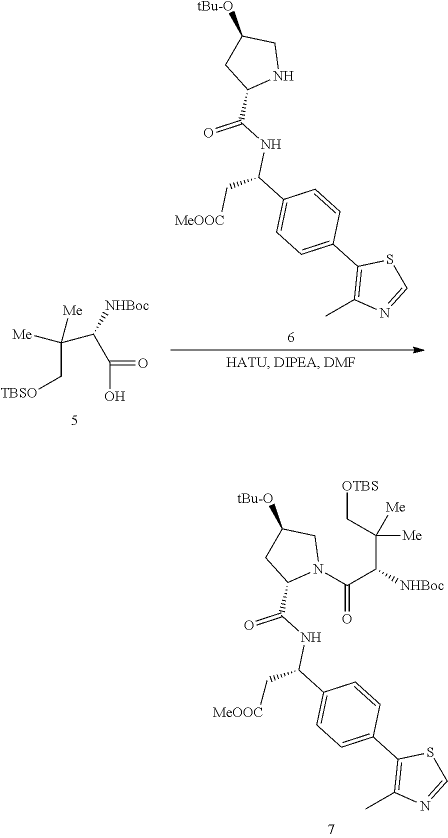

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 789.4, found:

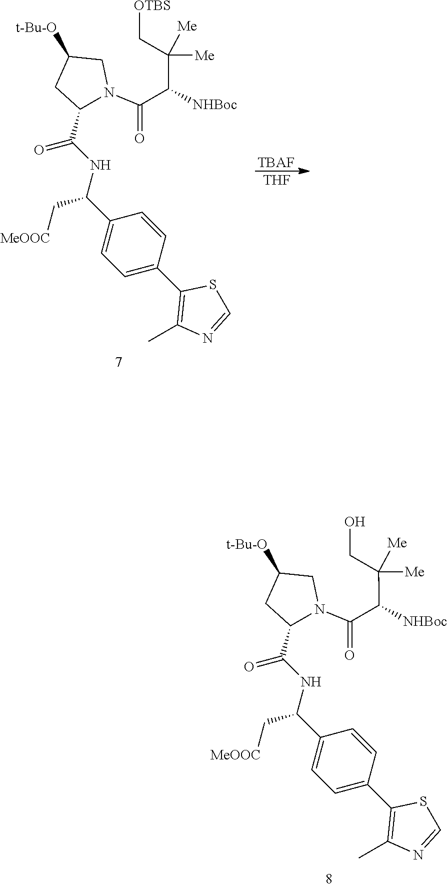

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+Na]⁺: 697.3, found: 697.0.

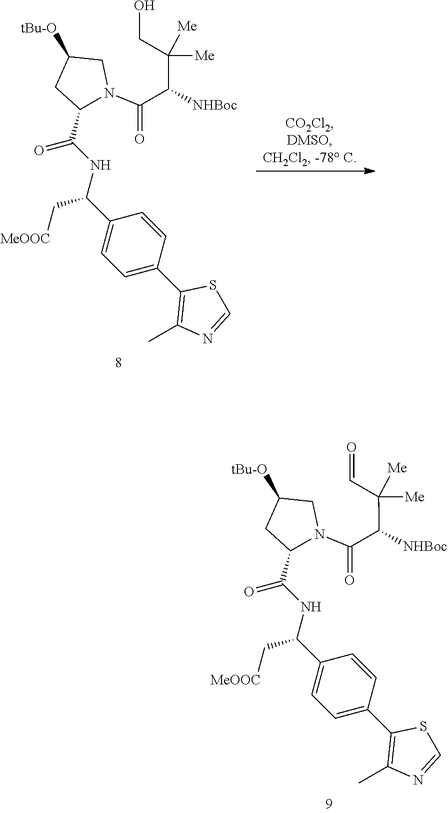

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 673.3, found: 673.3.

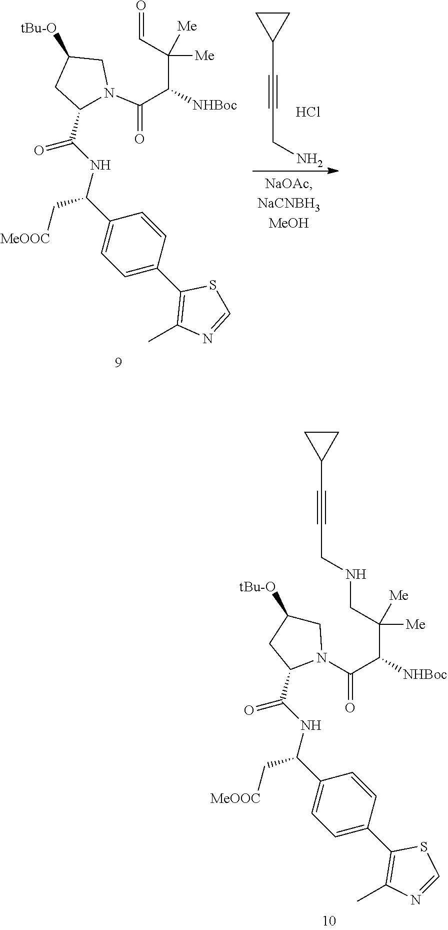

To a solution of 9 (1 equiv.) in MeOH. 3-cyclopropylprop-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 752.4, found: 752.8.

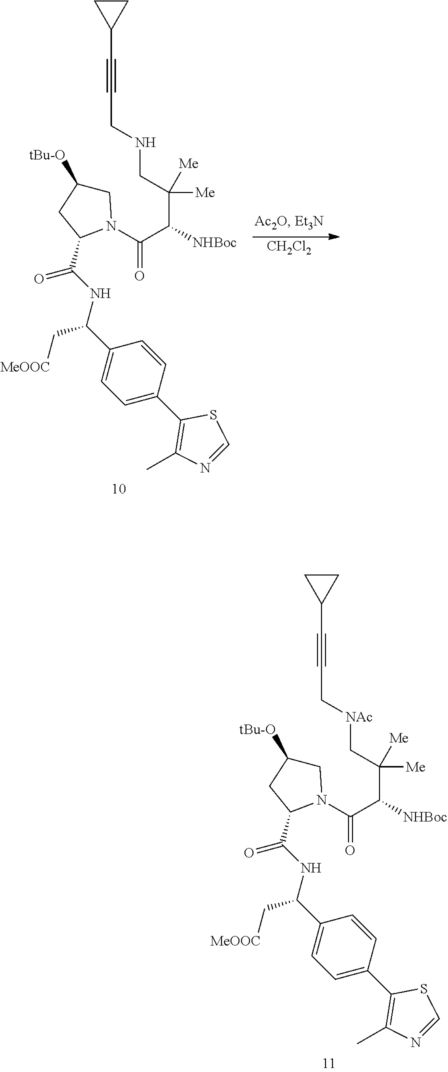

To a solution of 10 (1 equiv.) in DCM. Et₃N (3 equiv.) and Ac₂O (1.2 equiv.) were added. The reaction was stirred for 1 hours. The mixture was concentrated by vacuum and purified by silica column chromatography to afford 11. UPLC-MS: calculated [M+H]⁺: 794.4, found: 794.5.

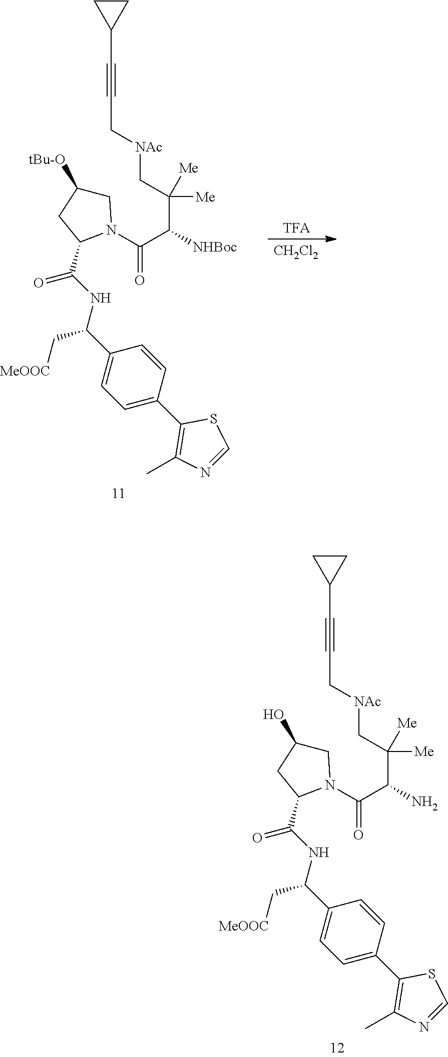

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 638.3, found: 638.4.

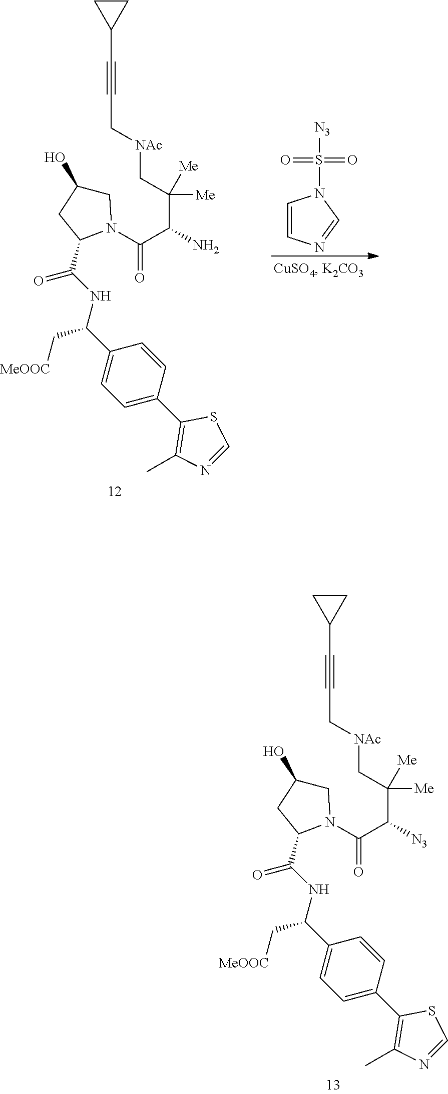

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 664.3, found: 664.3.

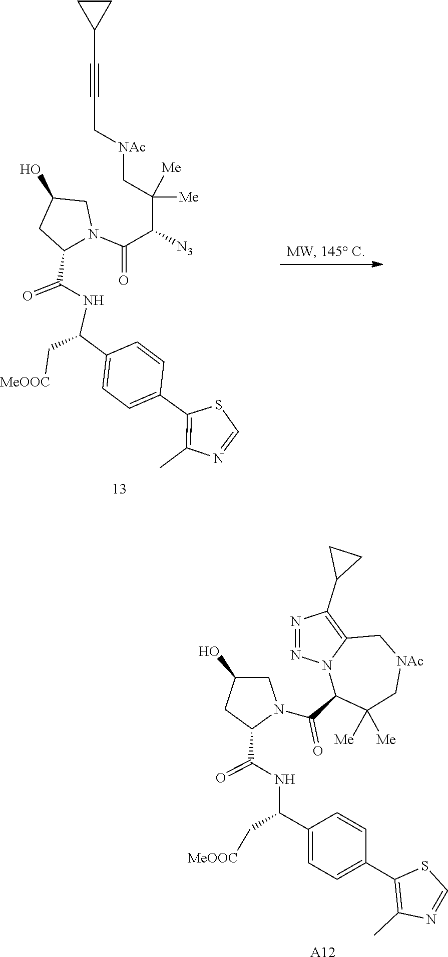

To a solution of 13 (1 equiv.) in 1,2-DCB (c₁₃=0.05 mol/L). Microwave the reaction at 145° C. for 16 h. The solvent was removed in vacuum for next step without further purification. UPLC-MS: calculated [M+H]⁺: 664.3, found: 664.1.

### Compound A13. methyl (S)-3-((2S,4R)-1-((7S,8S)-3-cyclopropyl-7-methyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanoate

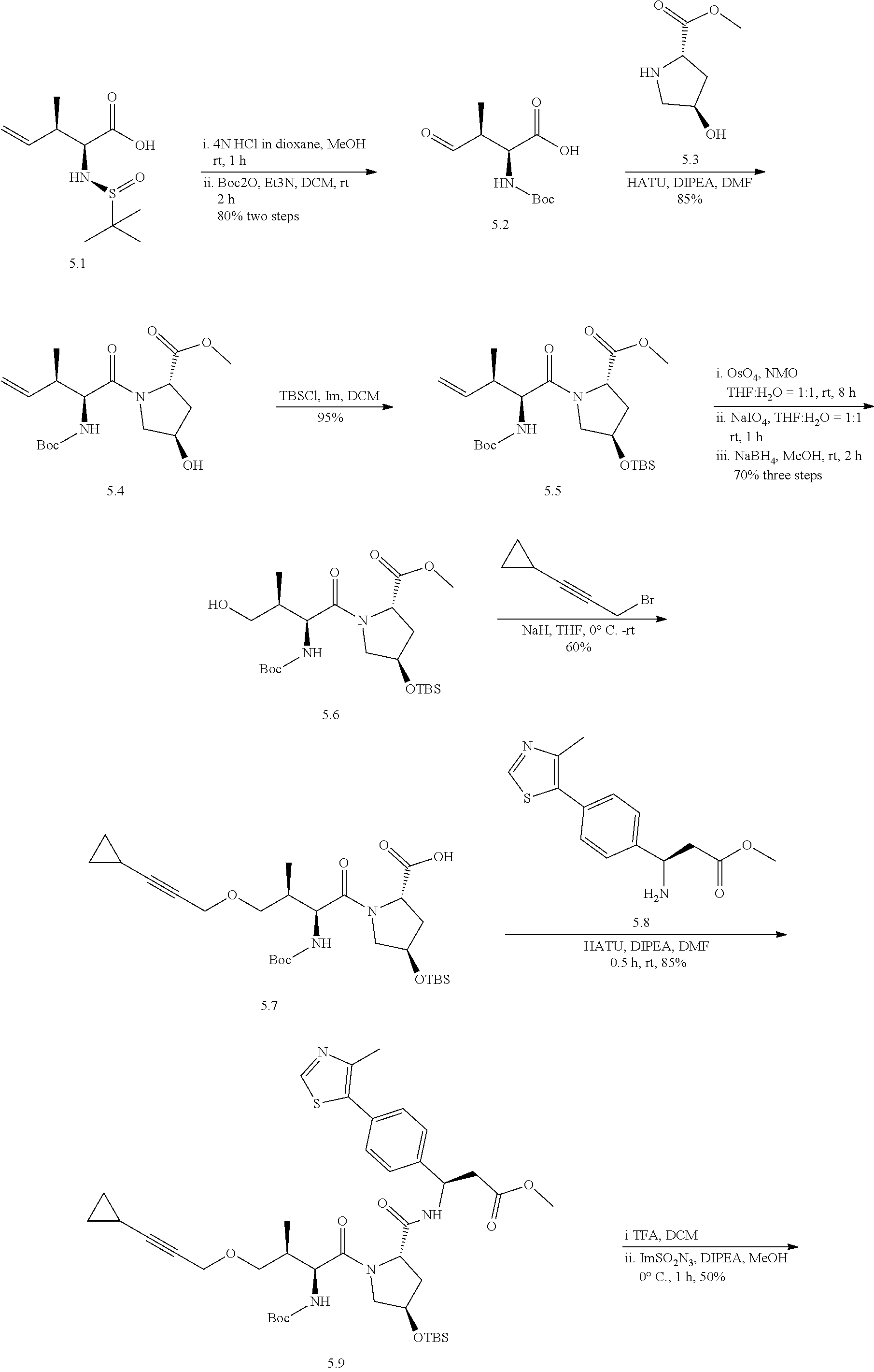

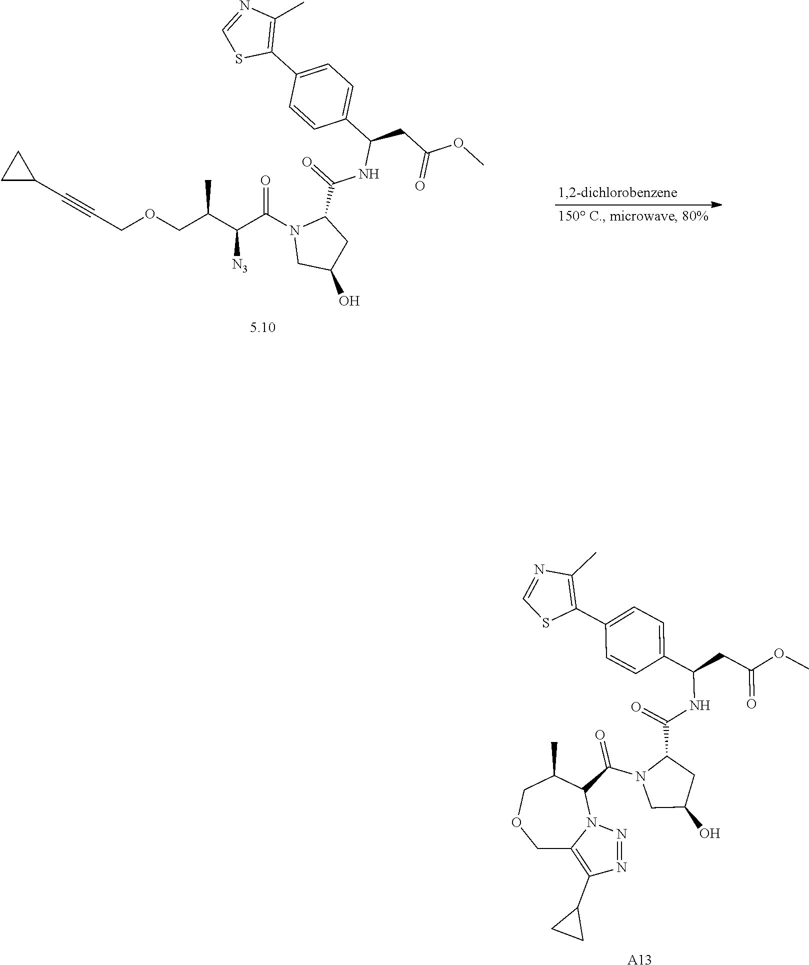

### Step 1

To a stirred solution of compound 5.1 (1 eq) in MeOH at rt, HCl (1.2 eq) in dioxane was added and the mixture was stirred for 1 h. After completion of the reaction (monitored by UPLC mass spectroscopy), the organic solvent was evaporated under reduced pressure and the crude product was used for the next step.

The crude material was dissolved in DCM and the mixture was cooled to 0° C. DIPEA (2 eq) and Boc anhydride (1.2 eq) were sequentially added to the reaction mixture and stirred for another 2 h at room temperature. After the complete conversion of the starting material, the mixture was concentrated and purified by flash column chromatography. UPLC-MS: calculated [M+H]⁺: 230.13, found: 230.15

### Step 2

To a stirred solution of Compound 5.2 (1 eq), 5.1 (1 eq), and HATU (1 eq) in DMF at 0° C., DIPEA was added dropwise, and the reaction mixture was stirred at room temperature for another 0.5 h. After completion of the reaction (monitored by UPLC), the mixture was diluted with water and extracted with ethyl acetate. The product was purified by flash column chromatography. UPLC-MS: calculated [M+H]⁺: 257.19, found: 256.92

### Step 3

To a stirred solution of compound 5.4 in DCM at room temperature, imidazole, and TBSCl were added, and the mixture was stirred at rt for 2 h. After complete conversion of the starting material (monitored by UPLC), the mixture was extracted with DCM and water. The organic layer was washed with anhydrous Na₂SO₄ and concentrated under reduced pressure. The product was purified with flash column chromatography. UPLC-MS: calculated [M+H]⁺: 471.28, found: 470.9

### Step 4

To a stirred solution of compound 5.5 in THF: Water (1:1) at rt, NMO (1.5 eq), and OsO₄ (0.1 eq) were added, and the mixture was stirred at rt overnight. After complete conversion of the starting material, the mixture was diluted with water and extracted with ethyl acetate. The organic layer was separated and evaporated under reduced pressure. The crude product was used for the next step.

The crude material was dissolved in THF: Water (1:1) and NaIO₄ (1.5 eq) was added to the reaction mixture and stirred for 1 h. After completion of the reaction, the mixture was diluted with water and extracted with ethyl acetate. The organic layer was separated and washed with brine and dried over anhydrous Na₂SO₄. Organic layer was concentrated under reduced pressure and the crude product was used for the next step.

To a stirred solution of the crude aldehyde at 0° C., NaBH₄ (1.2 eq) was added portion-wise. After complete addition of the reagent, the reaction was stirred at rt for another 2 h. After complete conversion of the aldehyde, the reaction was quenched with water, and the organic solvent was evaporated under reduced pressure. The crude mixture was extracted with ethyl acetate. The product was purified using flash column chromatography. UPLC-MS: calculated [M+H]⁺: 475.28, found: 475.04

### Step 5

To a suspension of NaH (1.5 eq) in THF at 0° C., compound 5.6 was added and stirred for 30 min. (3-bromoprop-1-yn-1-yl)cyclopropane was then added to the mixture in THF dropwise. The reaction mixture was allowed to warm at rt and stirred for overnight. After completion of the reaction, the mixture was diluted with ethyl acetate and quenched with a saturated solution of ammonium chloride. The mixture was extracted with ethyl acetate and dried over Na₂SO₄. The product was purified using flash column chromatography. UPLC-MS: calculated [M+H]⁺: 539.31, found: 539.00

### Step 6

To a stirred solution of compound 5.7, compound 5.8, and HATU in DMF at 0° C., DIPEA was added dropwise and stirred for 0.5 h at rt. After completion of the reaction, it was diluted with water and extracted with ethyl acetate. The crude product was purified using flash column chromatography. UPLC-MS: calculated [M+H]⁺: 797.39, found: 797.28

### Step 7

To a stirred solution of compound 5.9 in DCM at rt, TFA was added and stirred until starting material disappears. Upon completion of reaction, organic solvent and TFA was evaporated under reduced pressure and the crude material was used for the next step.

To a stirred solution of crude amine in MeOH at 0° C., DIPEA (1.5 eq) and CuSO₄ (0.1 eq) were added and stirred for 5 mins. ImSO₂N₃ was then added to the reaction mixture portion-wise at nitrogen atmosphere. The reaction was monitored by UPLC mass spectroscopy. Upon completion of the reaction, solvent was evaporated under reduced pressure and the product was purified using reverse phase column chromatography. The product was lyophilized with ACN: H₂O (1:2) mixture. UPLC-MS: calculated [M+H]⁺: 609.24, found: 608.85 Step 8:

A solution of compound 5.10 in 1,2-dichlorobenzene in a microwave tube was microwaved for 2 days at 150° C. and monitored by UPLC chromatography. Upon complete conversion of the starting material, organic solvent was evaporated under reduces pressure and the product was purified using preparative HPLC (ACN:H₂O with 0.1% TFA). The product was lyophilized and used for next steps. UPLC-MS: calculated [M+H]⁺: 609.24, found: 608.95

### Compound A14. methyl (S)-3-((2S,4R)-1-((7R,8S)-5-benzyl-3,7-dimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanoate

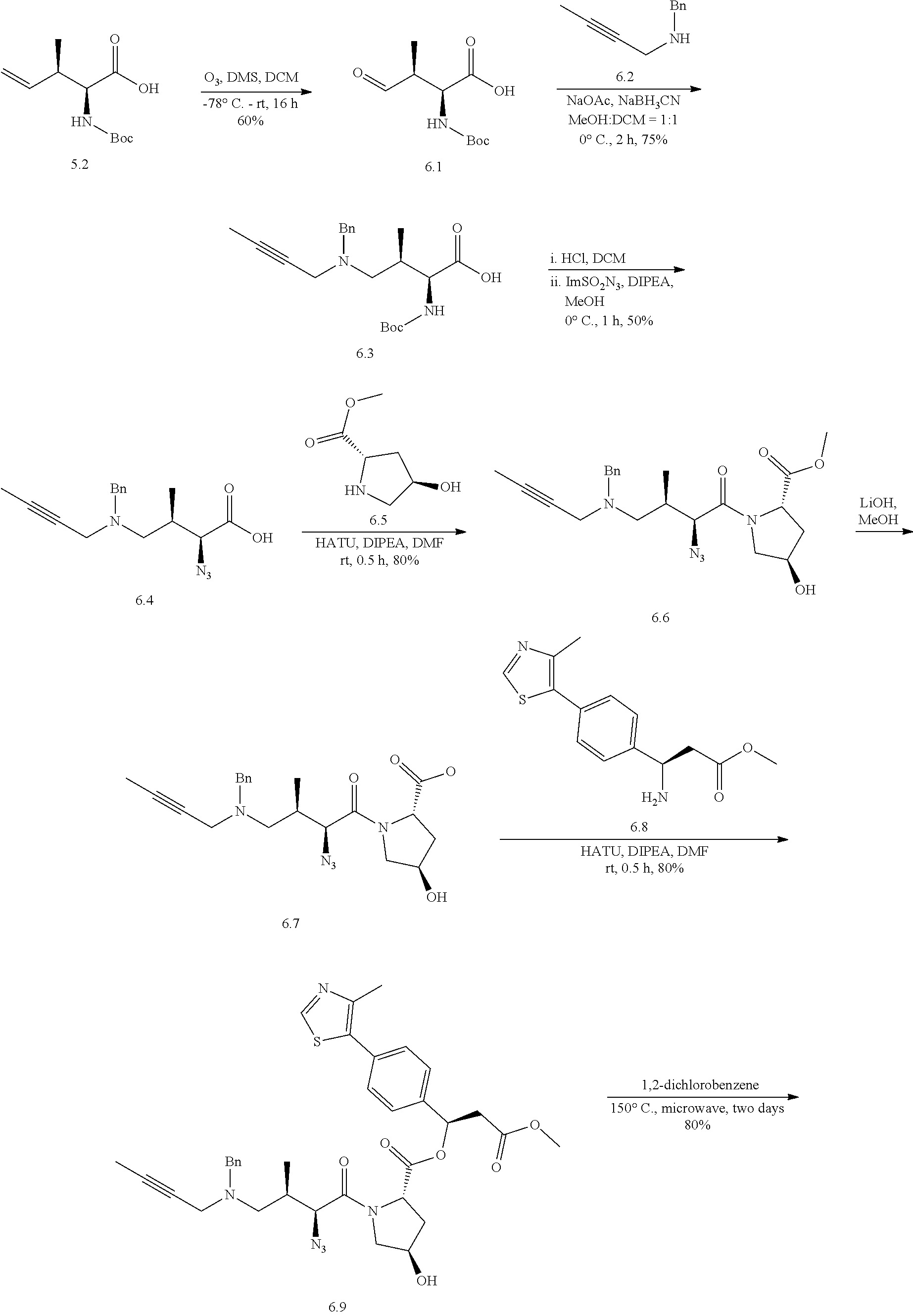

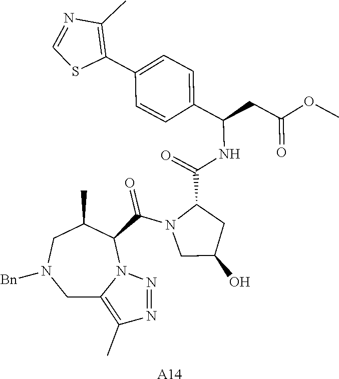 [SMILES: COC(=O)C[C@@H](NC(=O)[C@@H]1C[C@@H](O)CN1C(=O)[C@@H]1[C@H](C)CN(Cc2ccccc2)Cc2c(C)nnn21)c1ccc(-c2scnc2C)cc1 ; "A14"]

### Step 1

To a stirred solution of compound of compound 5.2 in DCM at −78° C., ozone gas was passed for 0.5 h and monitored by TLC analysis. After the full conversion of the starting material, DMS (10 eq) was added at −78° C. and the reaction was allowed to warm at rt and stirred for 16 h. The solvent was evaporated under reduced pressure and the product was purified using flash column chromatography. UPLC-MS: calculated [M+H]⁺: 232.11, found: 232.16.

### Step 2

To a stirred solution of aldehyde 6.1 (1 eq), amine 6.2 (1.2 eq), and sodium acetate (2.5 eq) in MeOH:DCM (1:1) at 0° C., NaBH₃CN (2 eq) was added and the mixture was stirred at room temperature for another 2 h. After completion of the reaction, solvent was evaporated under reduced pressure and the product was purified using reverse phase column chromatography. UPLC-MS: calculated [M+H]⁺: 375.22, found: 375.03.

### Step 3

To a stirred solution of compound 6.3 in DCM at rt, HCl in dioxane was added and stirred until starting material disappears. Upon completion of reaction, organic solvent and HCL was evaporated under reduced pressure and the crude material was used for the next step.

To a stirred solution of crude amine in MeOH at 0° C., DIPEA (1.5 eq) and CuSO₄ (0.1 eq) were added and stirred for 5 mins. ImSO₂N₃ was then added to the reaction mixture portion-wise at nitrogen atmosphere. The reaction was monitored by UPLC mass spectroscopy. Upon completion of the reaction, solvent was evaporated under reduced pressure and the product was purified using reverse phase column chromatography. The product was lyophilized with ACN:H₂O (1:2) mixture. UPLC-MS: calculated [M+H]⁺: 301.16, found: 301.08.

### Step 4

To a stirred solution of Compound 6.4 (1 eq), 6.5 (1 eq), and HATU (1 eq) in DMF at 0° C., DIPEA (1.5 eq) was added dropwise, and the reaction mixture was stirred at room temperature for another 0.5 h. After completion of the reaction (monitored by UPLC), the mixture was diluted with water and extracted with ethyl acetate. The product was purified by flash column chromatography. UPLC-MS: calculated [M+H]⁺: 428.22, found: 428.08.

### Step 5

To a stirred solution of compound 6.6 (1 eq) in MeOH:H₂O at rt, LiOH (2 eq) was added and stirred for 1 h. After completion of the reaction, mixture was acidified with 2 N HCl up to pH 3 and organic solvent was evaporated under reduced pressure. The mixture was extracted with ethyl acetate and purified with flash column chromatography. UPLC-MS: calculated [M+H]⁺: 414.21, found: 413.96.

### Step 6

To a stirred solution of Compound 6.7 (1 eq), 6.8 (1 eq), and HATU (1 eq) in DMF at 0° C., DIPEA (1.5 eq) was added dropwise, and the reaction mixture was stirred at room temperature for another 0.5 h. After completion of the reaction (monitored by UPLC), the mixture was diluted with water and extracted with ethyl acetate. The product was purified by flash column chromatography. UPLC-MS: calculated [M+H]⁺: 672.29, found: 671.96.

### Step 7

A solution of compound 6.9 in 1,2-dichlorobenzene in a microwave tube was microwaved for 2 days at 150° C. and monitored by UPLC mass chromatography. Upon complete conversion of the starting material, organic solvent was evaporated under reduces pressure and the product was purified using preparative HPLC (ACN:H₂O with 0.1% TFA). The product was lyophilized and used for next steps. UPLC-MS: calculated [M+H]⁺: 672.29, found: 671.94.

### Compound A15. methyl (S)-3-((2S,4R)-1-((S)-5-acetyl-3,7,7-trimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanoate

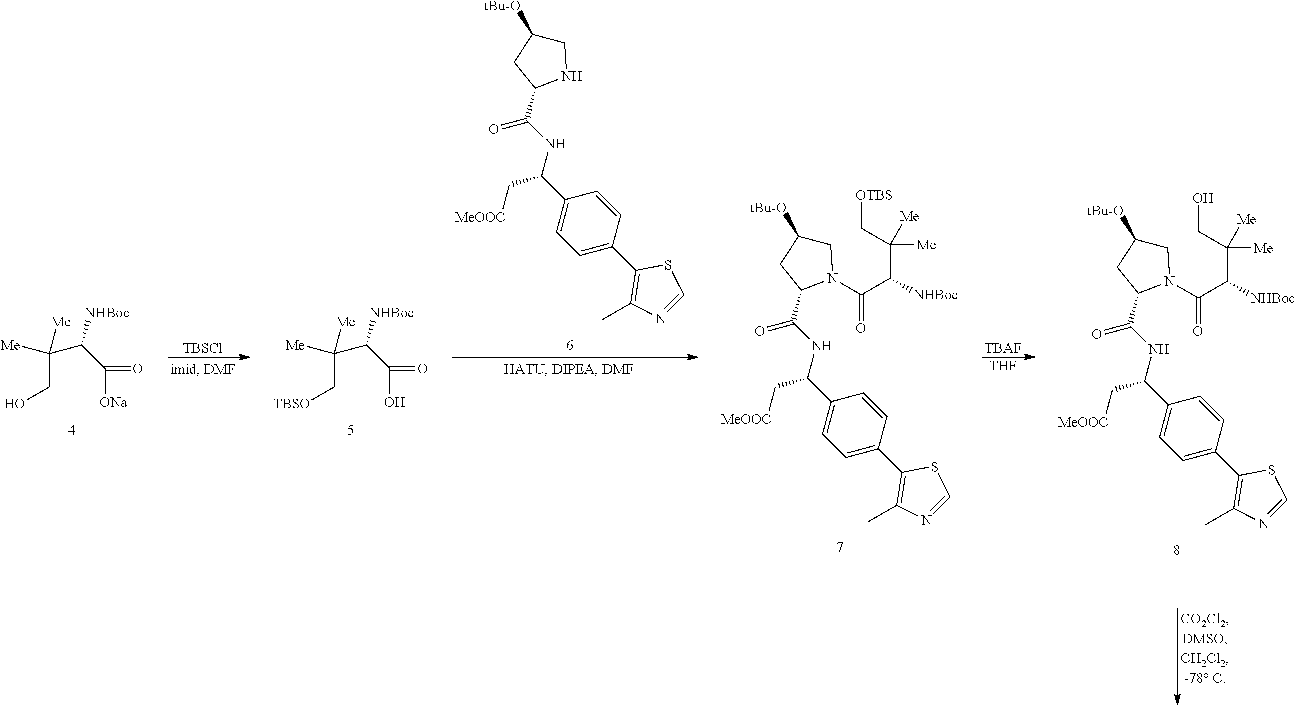

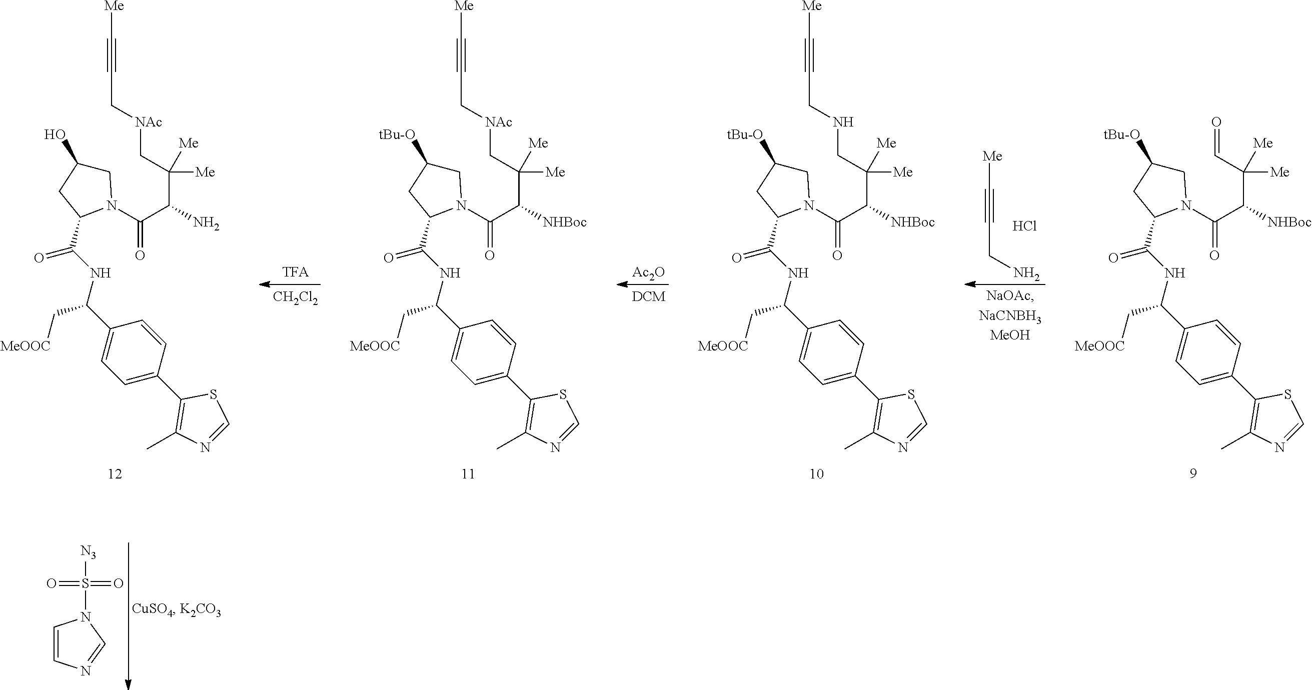

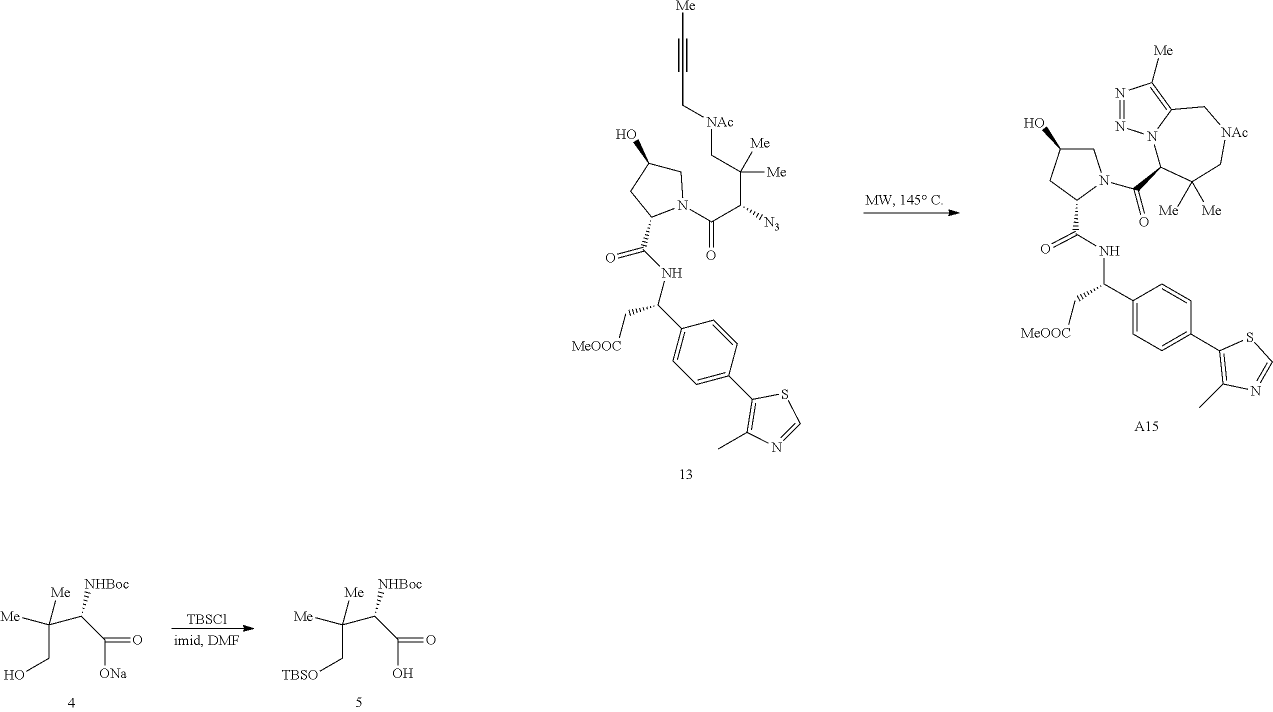

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

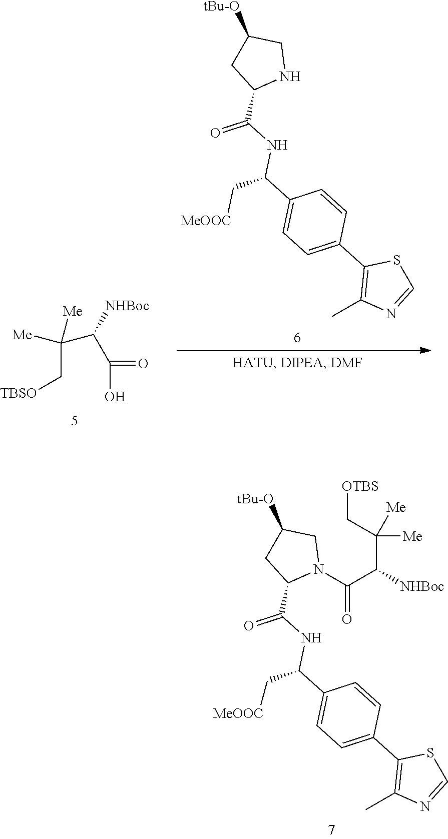

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 789.4, found: 788.9.

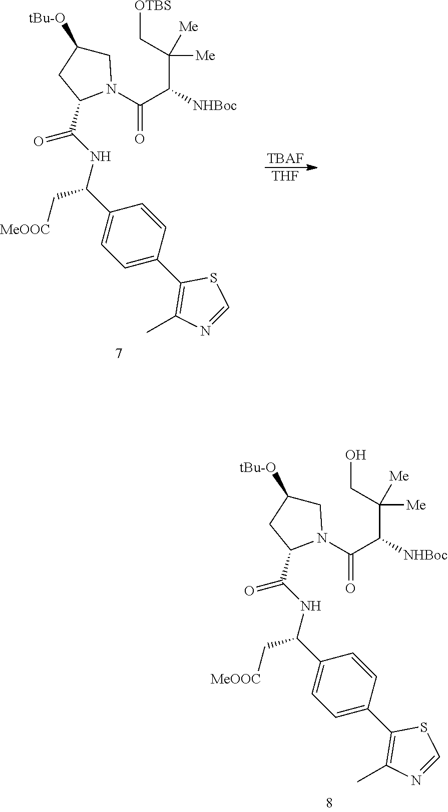

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+Na]⁺: 697.3, found: 697.0.

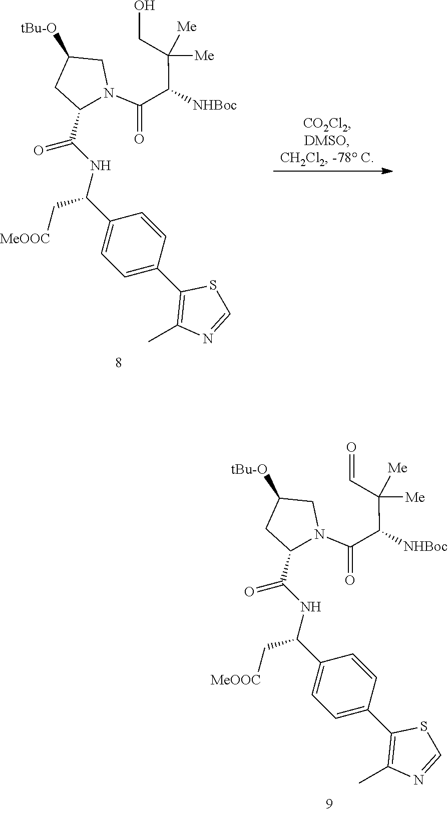

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 673.3, found: 673.3.

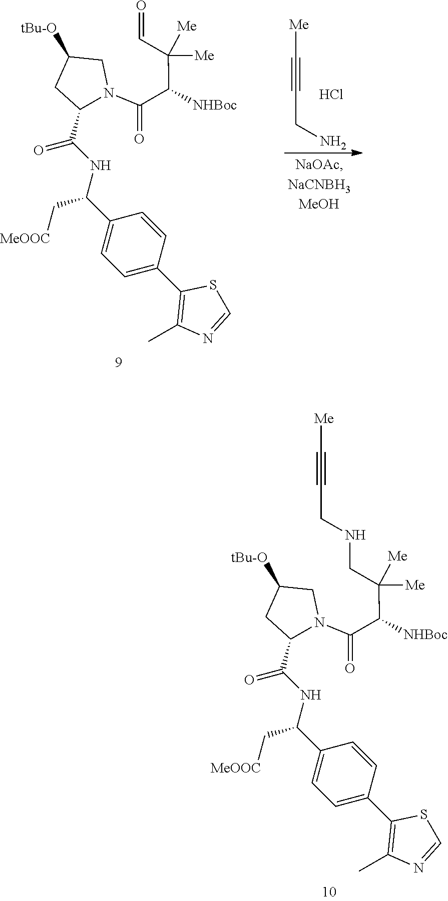

To a solution of 9 (1 equiv.) in MeOH. but-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 726.4, found: 726.2.

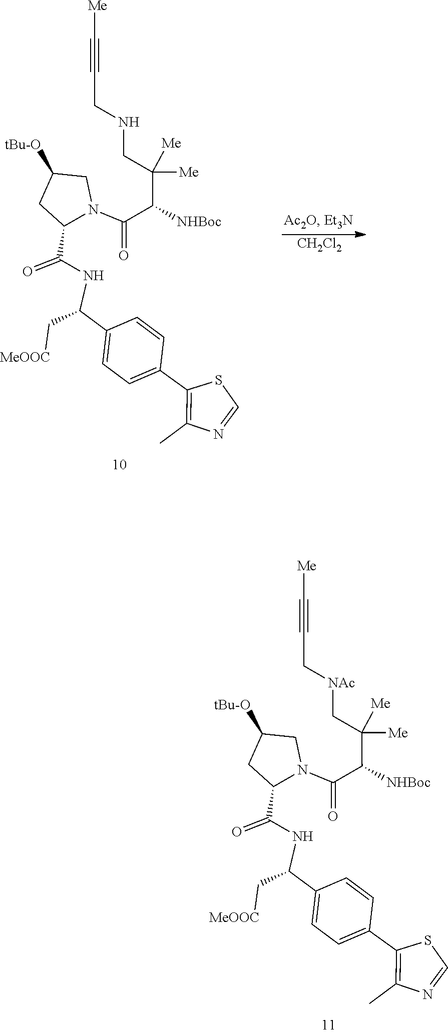

To a solution of 10 (1 equiv.) in DCM. Et₃N (3 equiv.) and Ac₂O (1.2 equiv.) were added. The reaction was stirred for 1 hours. The mixture was concentrated by vacuum and purified by silica column chromatography to afford 11. UPLC-MS: calculated [M+H]⁺: 768.4, found: 768.5.

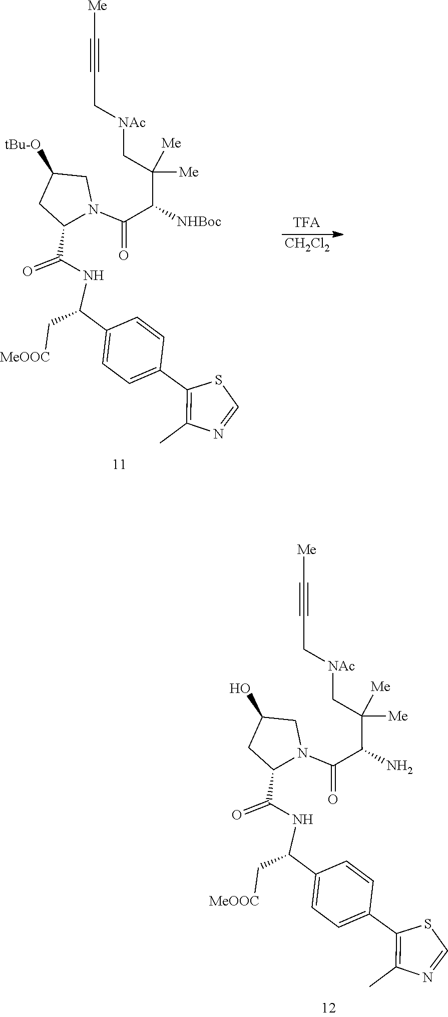

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 612.3, found: 612.3.

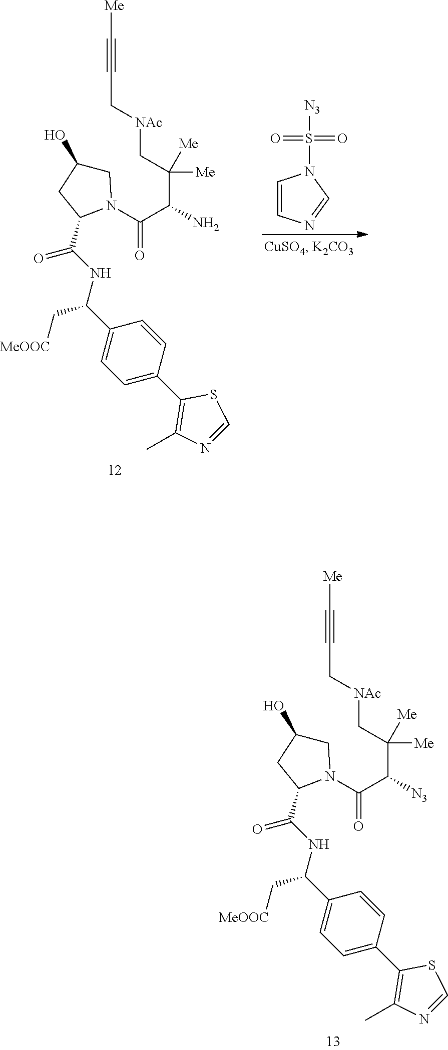

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 638.3, found: 638.1.

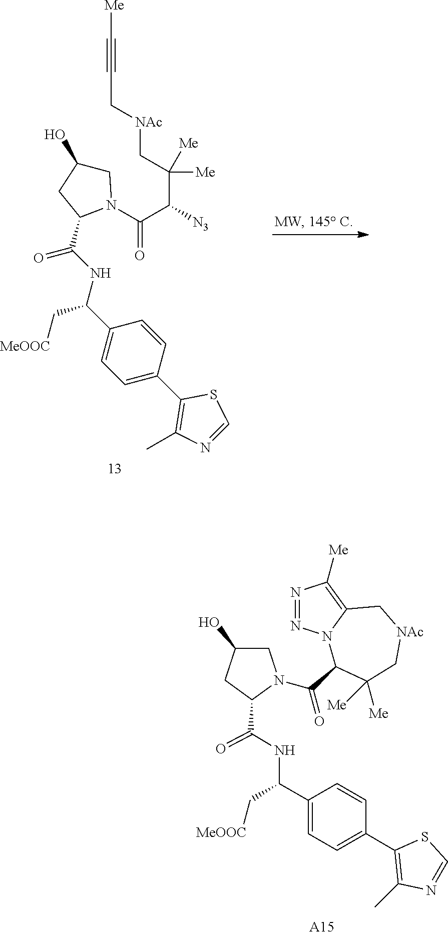

To a solution of 13 (1 equiv.) in 1,2-DCB (c₁₃=0.05 mol/L). Microwave the reaction at 145° C. for 16 h. The solvent was removed in vacuum for next step without further purification. UPLC-MS: calculated [M+H]⁺: 638.3, found: 638.3.

### Compound A16. ethyl (R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-5,6,7,8-tetrahydro-4H-cyclohepta[d]isoxazole-3-carboxylate

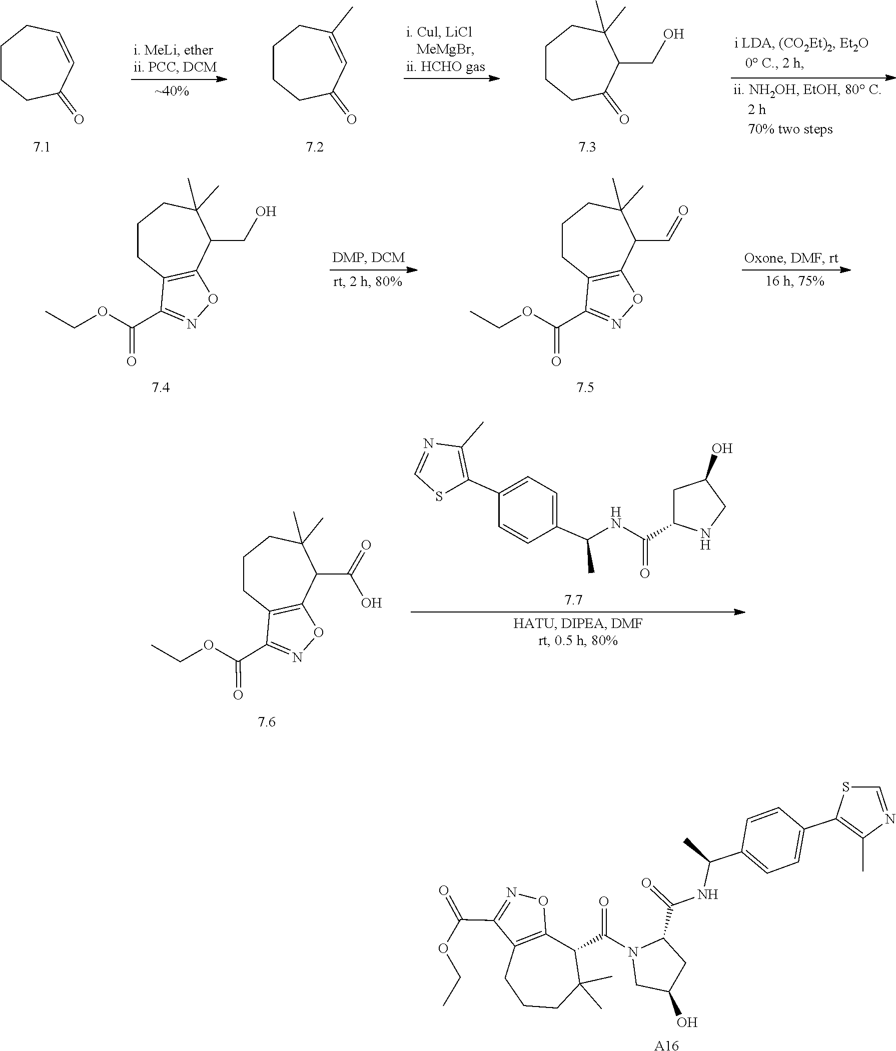

### Step 1

To a stirred solution of compound 7.1 in diethyl ether at −78° C., MeLi (1.1 eq) was added dropwise and the reaction was stirred at the same temperature for another 2 h. After complete conversion of the starting material, the reaction was quenched with saturated ammonium chloride and extracted with diethyl ether. The organic layer was washed with brine and dried over Na₂SO₄. The crude product was used for the next step.

To a stirred solution of the crude product in DCM at rt, PCC (1.5 eq) was added and the reaction was vigorously stirred for 2 h. The reaction was monitored by TLC chromatography. After complete conversion of the starting material, the reaction was diluted with ethyl ether and filtered through a pad of celite. The mixture was washed with ethyl ether. The filtrate was concentrated under reduced pressure and the product was purified with flash column chromatography. ¹H NMR (400 MHz, CDCl₃) δ 5.95 (d, J=1.9 Hz, 1H), 2.67-2.52 (m, 2H), 2.43 (dd, J=6.9, 4.6 Hz, 2H), 1.98 (dd, J=1.3, 0.7 Hz, 3H), 1.90-1.73 (m, 4H).

### Step 2

LiCl (0.1 eq) and CuI (0.05 eq) were added to a flame-dried two-way round bottom flask and purged with nitrogen three times. THF was added to the mixture and stirred vigorously until a homogeneous light-yellow solution. Compound 7.2 was added, and the reaction was cooled to 0° C. Methyl magnesium bromide (1.2 eq) was added to the reaction dropwise over 20 mins and the reaction was stirred at the same temperature for 2 h and monitored by TLC chromatography. After the complete conversion of starting material, the reaction was cooled to −10° C., and formaldehyde gas (generated by heating paraformaldehyde at 140° C.) was passed through the solution using a cannula for 4 h. The reaction was monitored by TLC chromatography. Upon completion of the reaction, the mixture was quenched with a saturated solution of ammonium chloride and extracted with ethyl acetate. The product was purified by flash column chromatography. ¹H NMR (400 MHz, CDCl₃) δ 4.02 (dd, J=11.2, 9.0 Hz, 1H), 3.71 (dd, J=11.2, 3.6 Hz, 1H), 2.96 (dd, J=9.0, 3.6 Hz, 1H), 2.67 (s, 2H), 2.59-2.34 (m, 2H), 1.89-1.73 (m, 3H), 1.66-1.40 (m, 2H), 1.05 (s, 3H), 0.83 (s, 3H).

### Step 3

A solution of compound 7.3 (1 eq), diethyl oxalate (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

A solution of crude product and hydroxyl amine hydrochloride (2 eq) in ethanol was heated to reflux for 2 h. After complete conversion of starting material, ethanol was evaporated under reduced pressure and the product was purified with flash column chromatography. ¹H NMR (400 MHz, CDCl₃) δ 4.41 (q, J=7.1 Hz, 2H), 3.81 (hept, J=6.7 Hz, 1H), 3.43 (hept, J=6.8 Hz, 1H), 3.14-3.03 (m, 1H), 2.98 (ddd, J=7.1, 5.9, 1.1 Hz, 1H), 2.60-2.44 (m, 1H), 2.06 (s, 2H), 1.88 (ddd, J=14.4, 11.1, 3.3 Hz, 1H), 1.71 (ddt, J=7.1, 6.0, 3.6 Hz, 1H), 1.61-1.53 (m, 1H), 1.40 (t, J=7.2 Hz, 3H), 1.05 (s, 3H), 0.93 (s, 3H). UPLC-MS: calculated [M+H]⁺: 268.15, found: 268.17.

### Step 4

To a stirred solution of compound 7.4 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography. ¹H NMR (400 MHz, CDCl₃) δ 9.85 (d, J=2.4 Hz, 1H), 4.41 (q, J=7.1 Hz, 2H), 3.76 (dd, J=2.4, 1.4 Hz, 1H), 3.16 (dddd, J=16.0, 5.2, 3.1, 1.2 Hz, 1H), 2.60-2.47 (m, 1H), 1.89 (ddd, J=14.0, 11.5, 2.7 Hz, 1H), 1.83-1.63 (m, 3H), 1.40 (t, J=7.1 Hz, 3H), 1.27 (s, 3H), 0.98 (s, 3H). UPLC-MS: calculated [M+H]⁺: 266.13, found: 266.07.

### Step 5

To a stirred solution of compound 7.5 (1 eq) in DMF at rt, Oxone monohydrate (3 eq) was added. The reaction was stirred at rt for 16 h followed by dilution with water. The mixture was extracted with ethyl acetate and the product was purified using flash column chromatography. UPLC-MS: calculated [M+H]⁺: 282.13, found: 282.04.

### Step 6

To a stirred solution of compound 7.6 (1 eq), compound 7.7 (1 eq), and HATU (1 eq) in DMF at rt, DIPEA (1.5 eq) was added dropwise and monitored by UPLC. After completion of the reaction, the product was purified by reverse phase column chromatography. UPLC-MS: calculated [M+H]⁺: 594.25, found: 595.10.

### Compound A17. methyl (S)-3-((2S,4R)-1-((S)-5-acetyl-3,8,8-trimethyl-4,5,6,7,8,9-hexahydro-[1,2,3]triazolo[1,5-a][1,4]diazocine-9-carbonyl)-4-hydroxypyrrolidine-2-carboxamido)-3-(4-(4-methylthiazol-5-yl)phenyl)propanoate

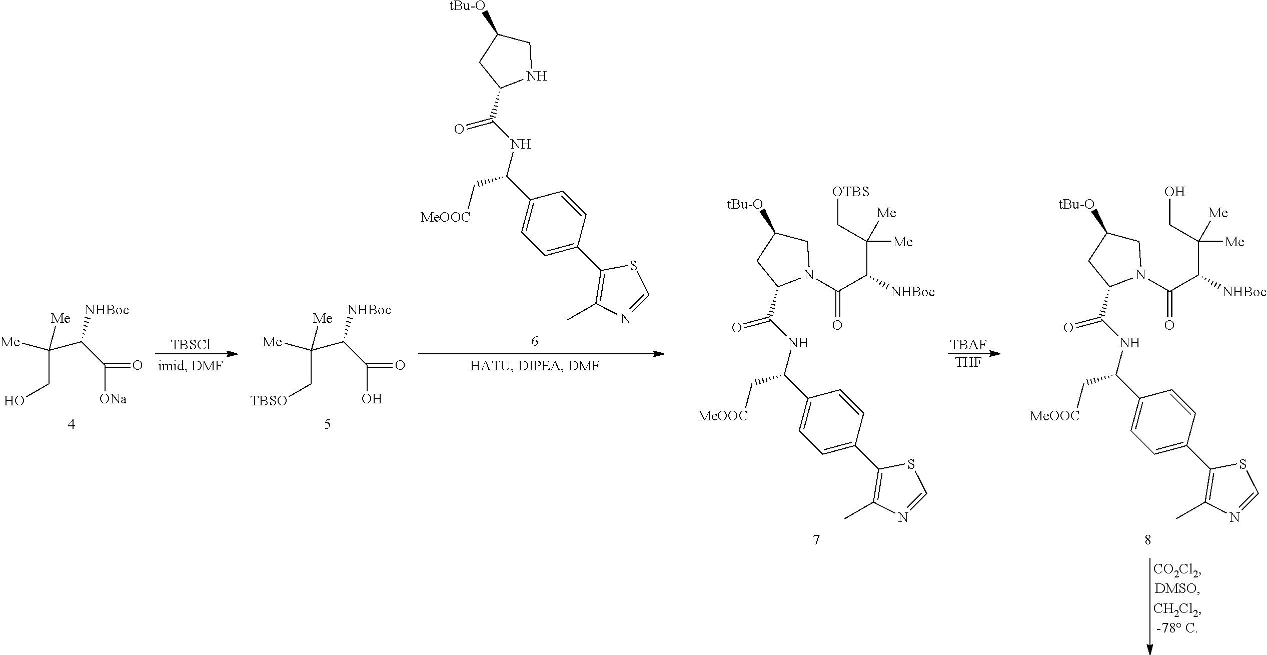

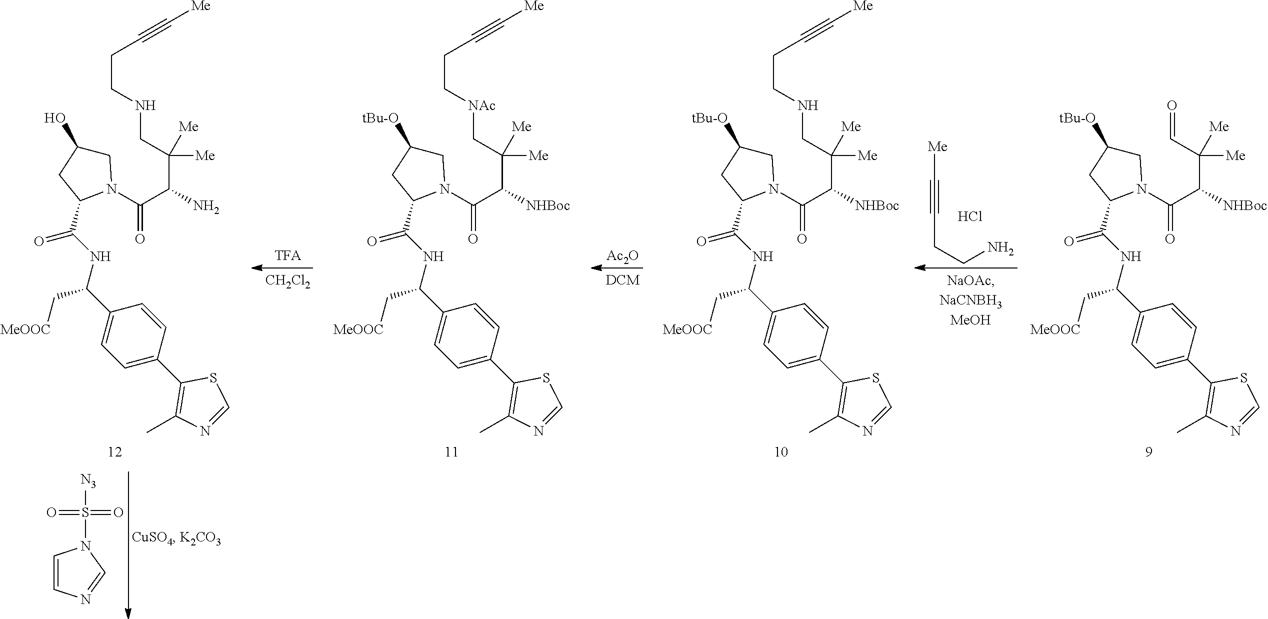

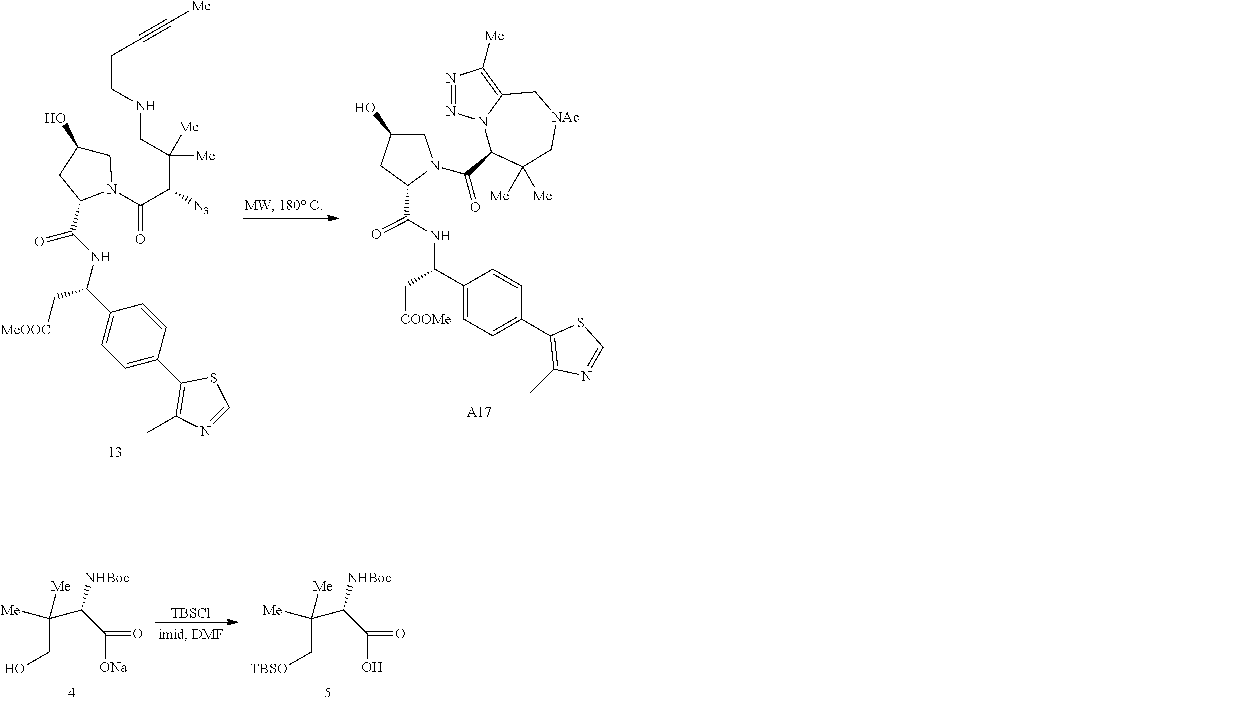

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

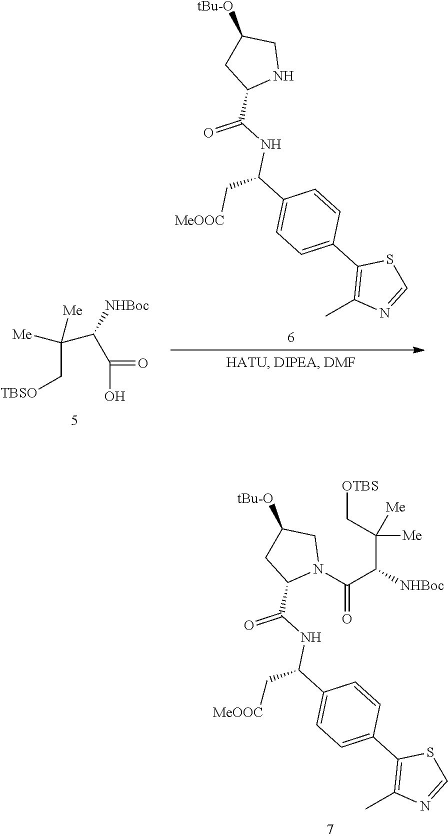

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 789.4, found: 788.9.

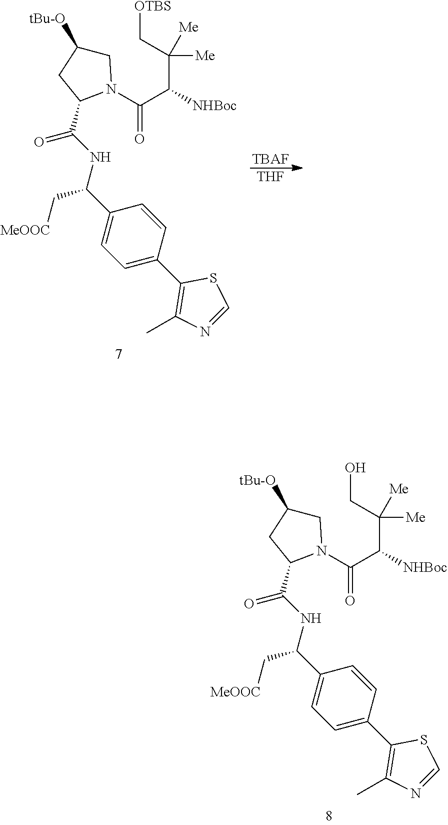

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+Na]⁺: 697.3, found: 697.0.

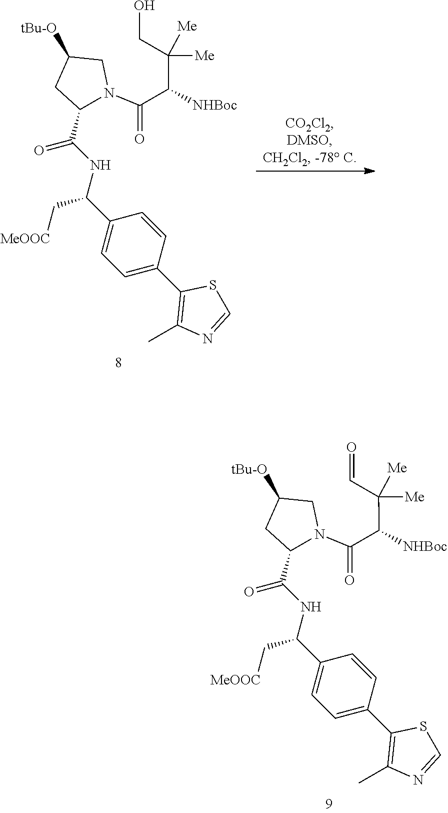

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 673.3, found: 673.3.

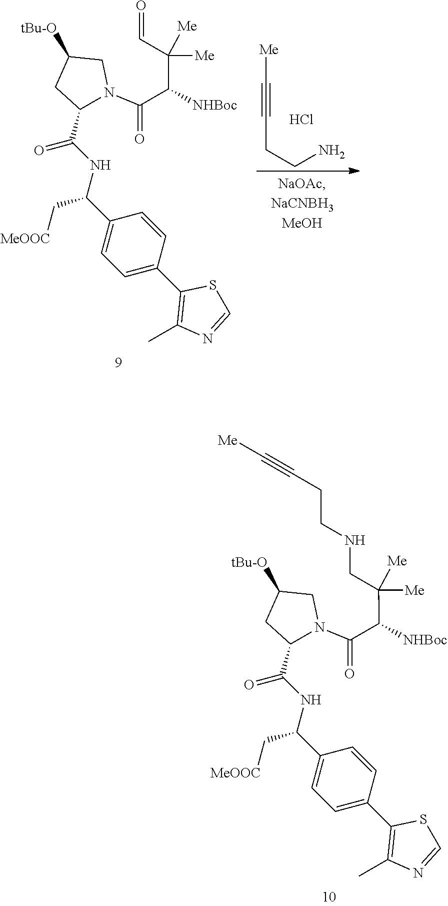

To a solution of 9 (1 equiv.) in MeOH. pent-3-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 740.4, found: 740.2.

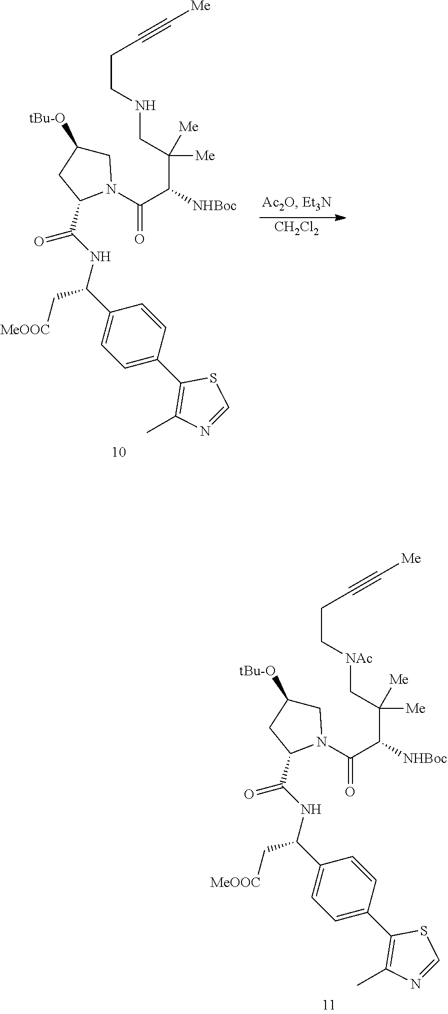

To a solution of 10 (1 equiv.) in DCM. Et₃N (3 equiv.) and Ac₂O (1.2 equiv.) were added. The reaction was stirred for 1 hours. The mixture was concentrated by vacuum and purified by silica column chromatography to afford 11. UPLC-MS: calculated [M+H]⁺: 782.4, found: 782.5.

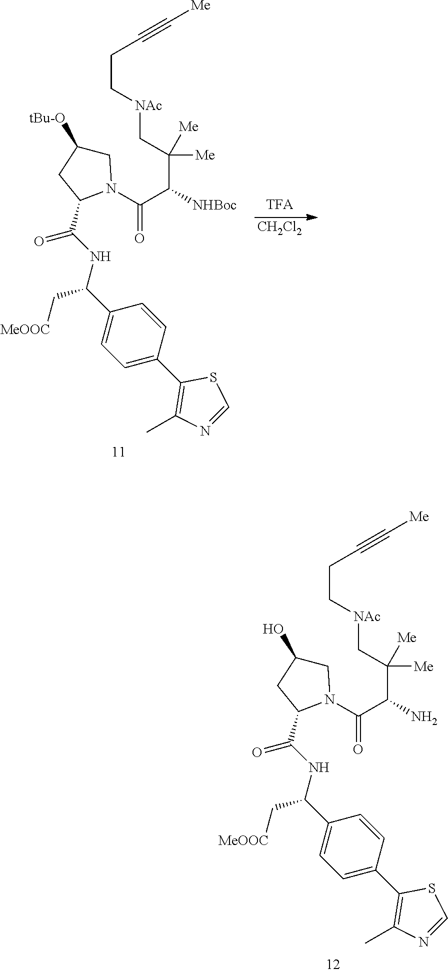

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 626.3, found: 626.4.

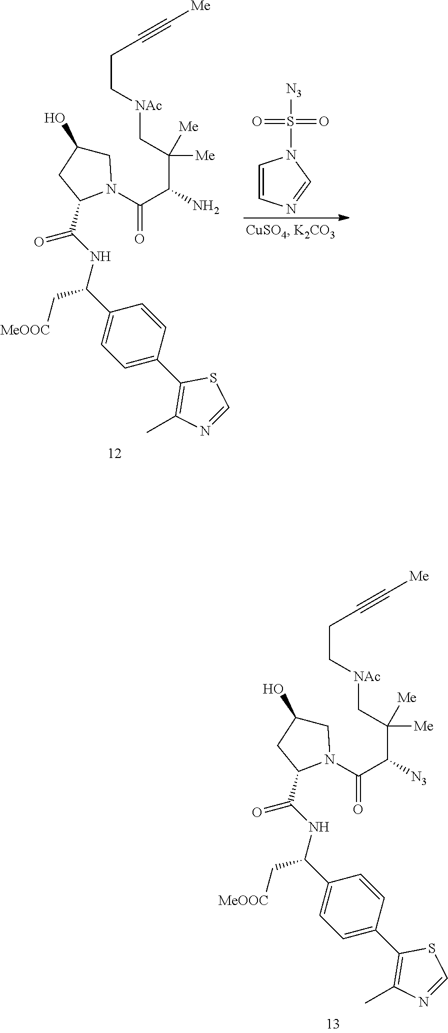

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 652.3, found: 652.1.

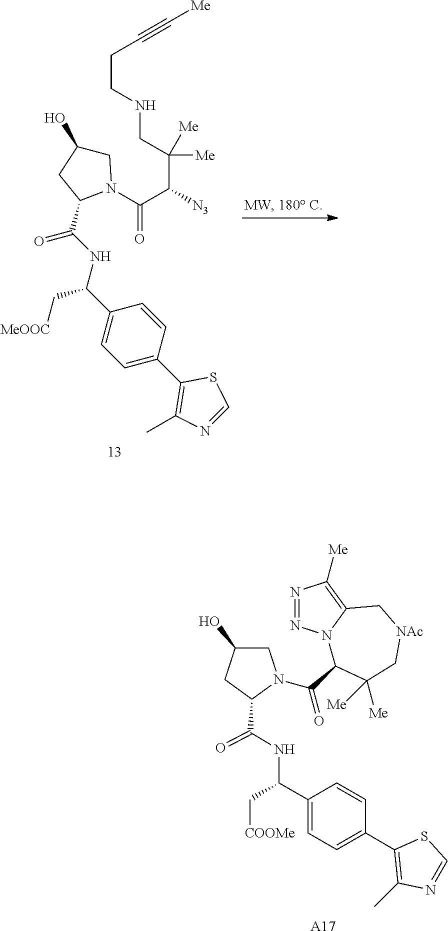

To a solution of 13 (1 equiv.) in 1,2-DCB (c₁₃=0.05 mol/L). Microwave the reaction at 180° C. for 30 h. The solvent was removed in vacuum for next step without further purification. UPLC-MS to provide A17: calculated [M+H]⁺: 652.3, found: 652.2.

### Compound A18. methyl (S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(2,4-dimethylthiazol-5-yl)phenyl)propanoate

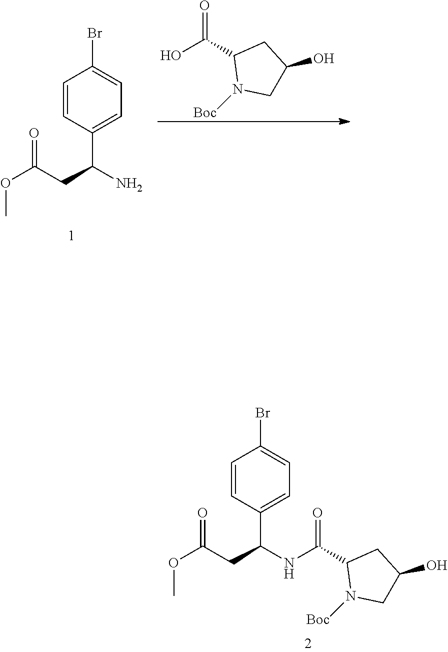

HATU (1.65 mmol, 1.1 equiv.) was added to a solution of 1 (1.5 mmol, 1 equiv.), (2S,4R)-1-(tert-butoxycarbonyl)-4-hydroxypyrrolidine-2-carboxylic acid (1.5 mmol, 1 equiv.) and DIEA (4.5 mmol, 3 equiv.) in DMF (15 mL) and the resulting mixture was stirred at rt for 1 h. This mixture was purified by reverse phase preparative HPLC to give 2. UPLC-MS calculated [M+H]+: 471.1, found: 471.3 Retention time: 3.8 min.

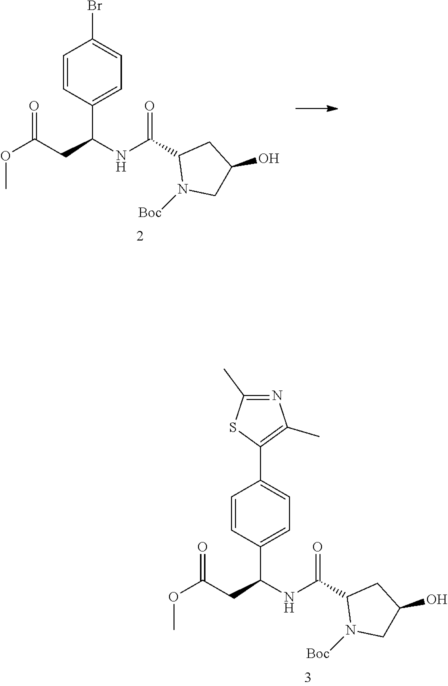

Pd(OAc)₂ (0.1 mmol, 0.11 equiv.) was added to a mixture of compound 2 (1.0 mmol, 1 equiv.), 2,4-dimethylthiazole (2 mmol, 2 equiv.) and Potassium acetate (2 mmol, 2 equiv) in DMF (5 mL). The resulting mixture was purged and refilled with argon three times and stirred at 100° C. overnight under Argon. The reaction mixture was then cooled to room temperature and evaporated to remove most of the solvent. The residue was purified by HPLC to yield the 3 UPLC-MS calculated [M+H]+: 504.2, found: 504.4. Retention time: 3.2 min.

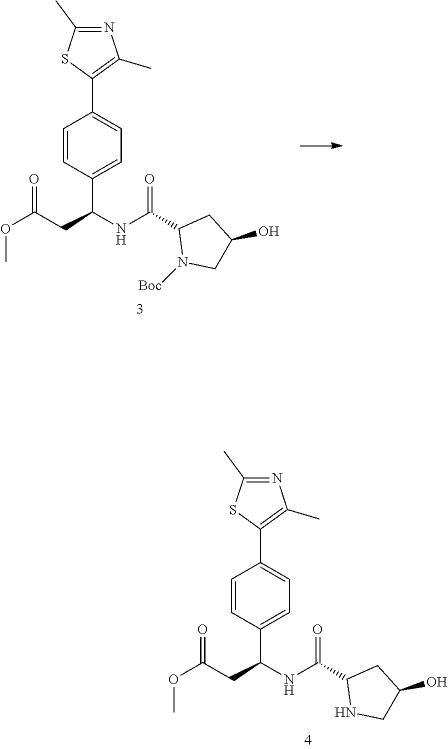

Compound 3 (250 mg) was treated with 5 mL of 1M HCl (in Methanol) and 2 mL of 4M HCl (in dioxane) for 2 h. The reaction mixture was evaporated to give the 4 without further purification. UPLC-MS calculated [M+H]⁺: 404.2, found: 404.5. Retention time: 1.8 min.

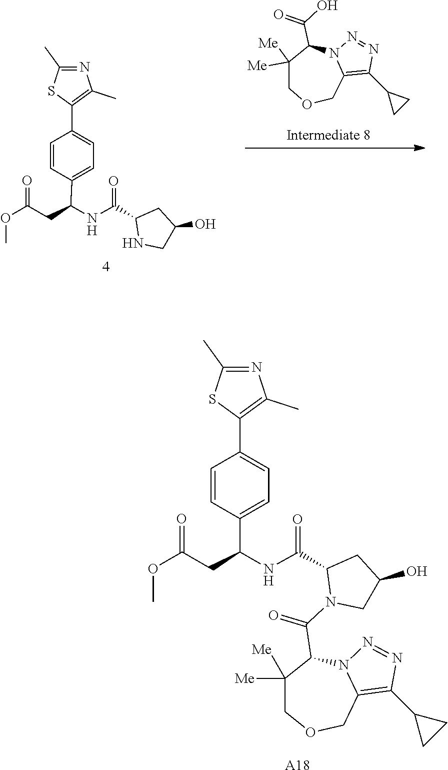

HATU (0.11 mmol, 1.1 equiv.) was added to a solution of Intermediate 8 (0.1 mmol, 1 equiv.), 4 (0.1 mmol, 1 equiv.) and DIEA (0.3 mmol, 3 equiv.) in DMF (1 mL) and the resulting mixture was stirred at rt for 1 h. This mixture was purified by reverse phase preparative HPLC to give A18. UPLC-MS calculated [M+H]⁺: 637.3, found: 637.6. Retention time: 3.6 min.

### Compound A19. methyl (S)-3-((2S,4R)-1-((S)-3-cyclopropyl-5,7,7-trimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanoate

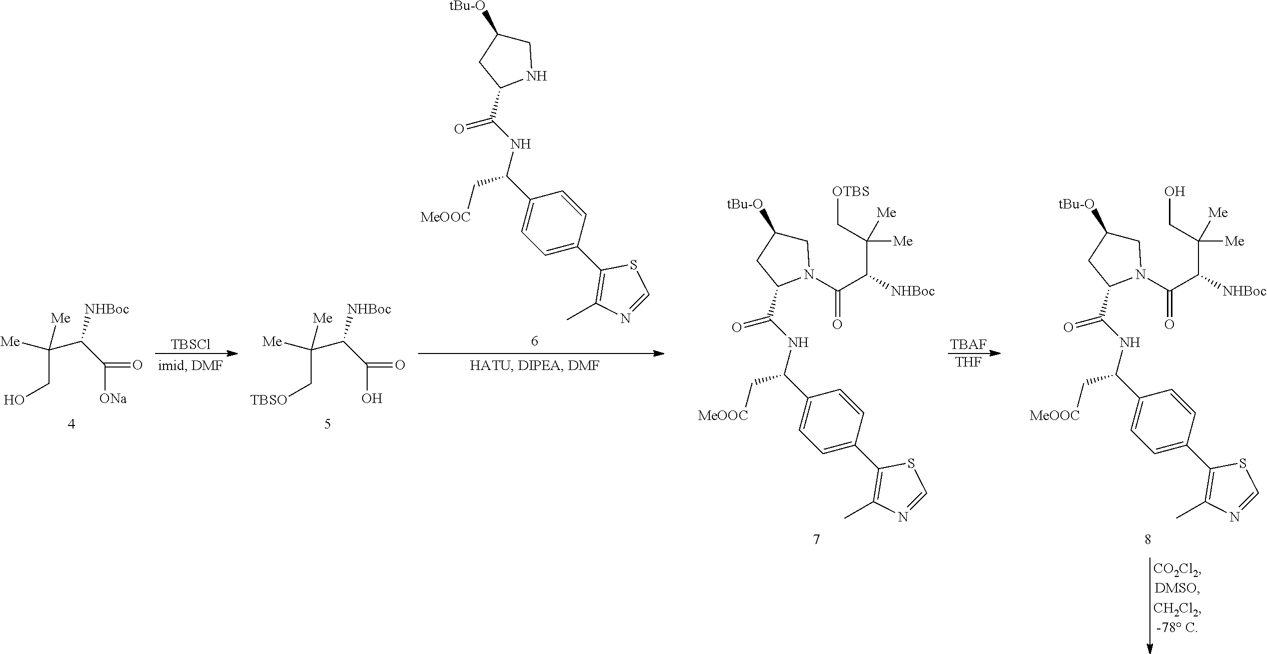

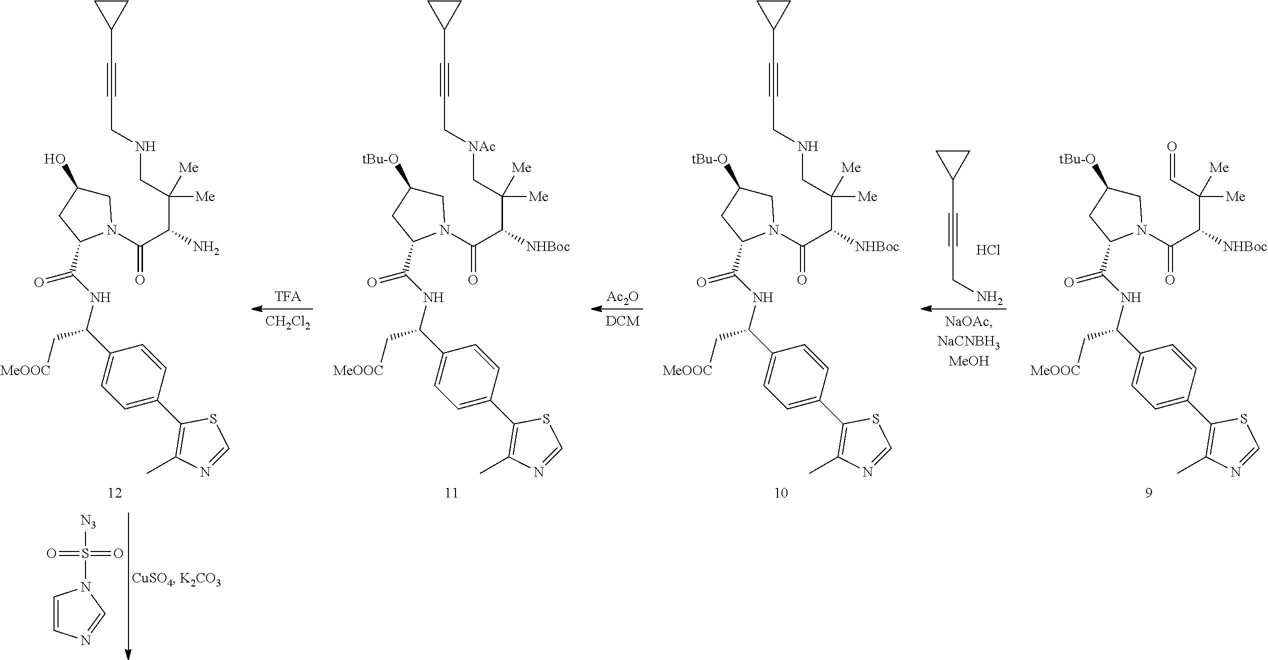

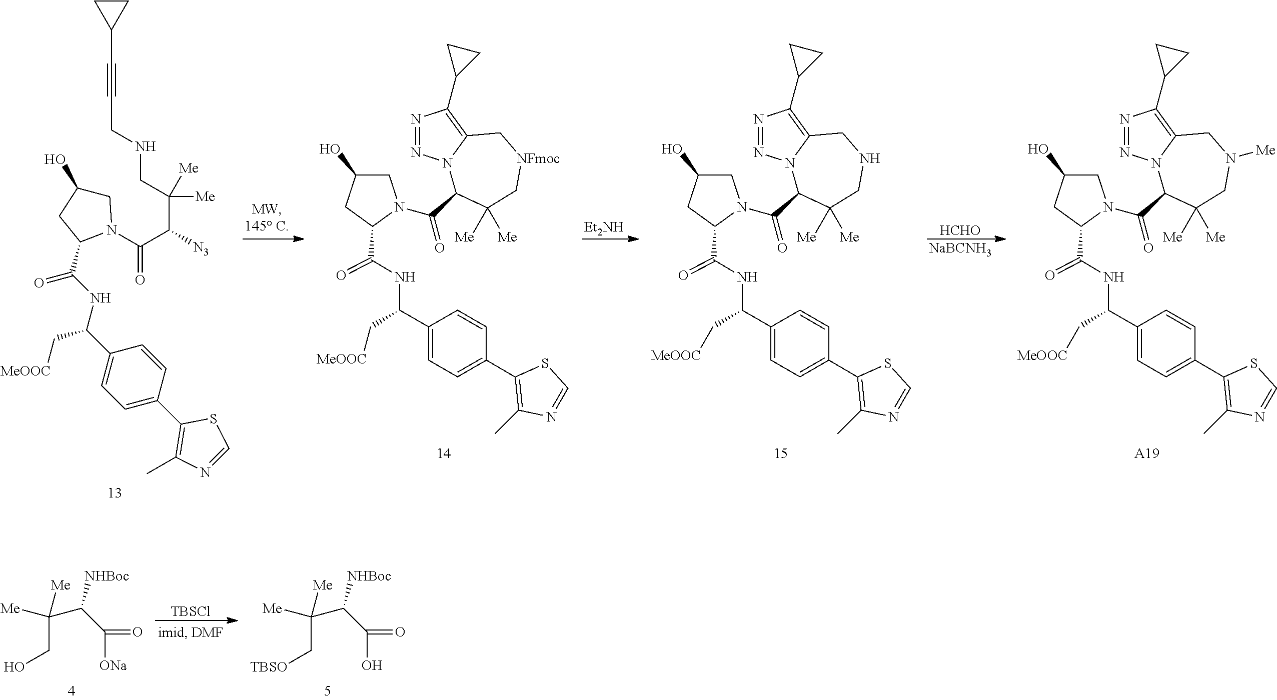

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

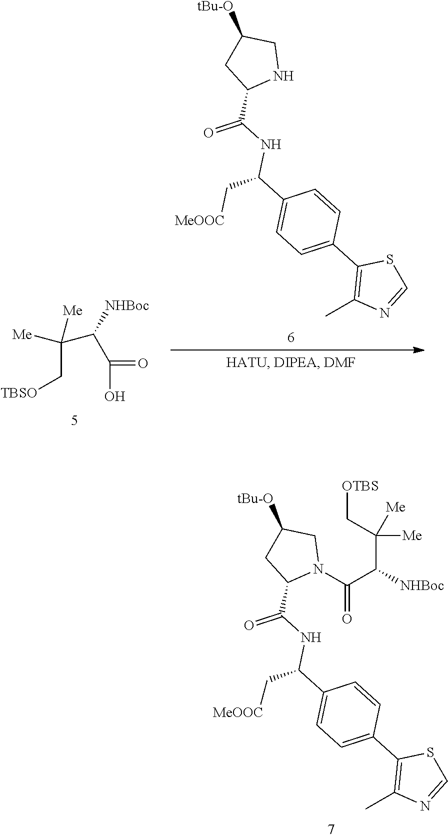

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 789.4, found: 788.9.

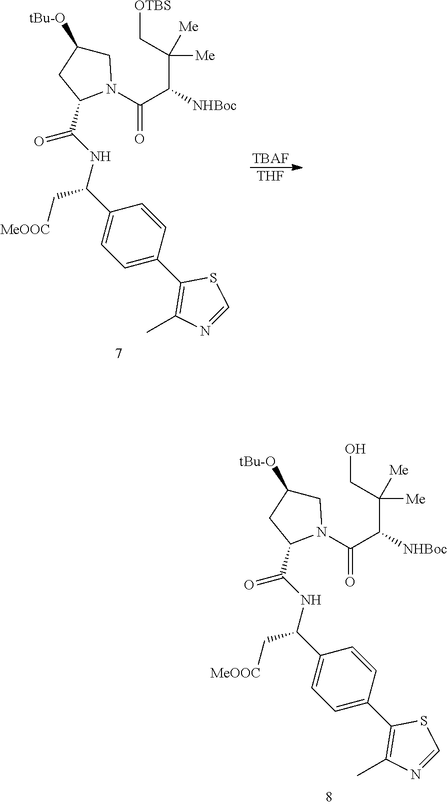

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+Na]⁺: 697.3, found: 697.0.

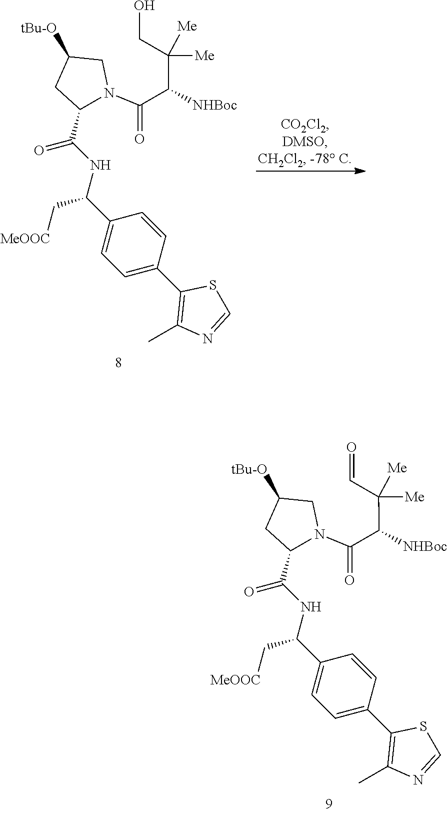

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 673.3, found: 673.3.

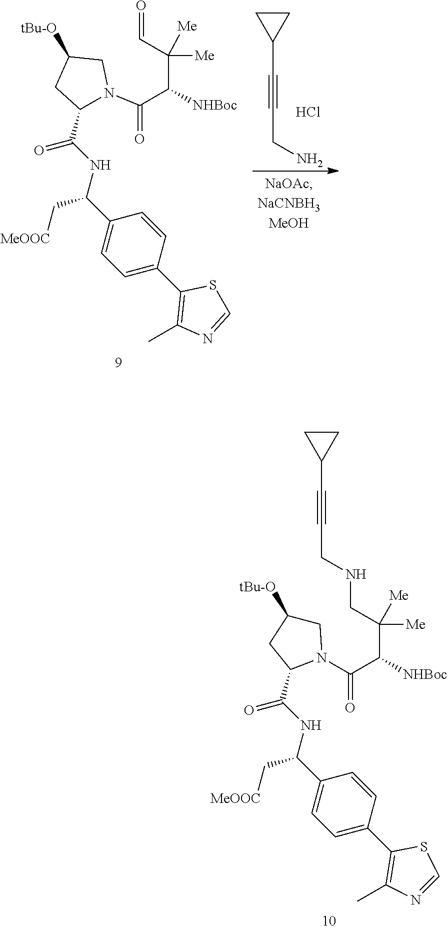

To a solution of 9 (1 equiv.) in MeOH. 3-cyclopropylprop-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 752.4, found: 752.8.

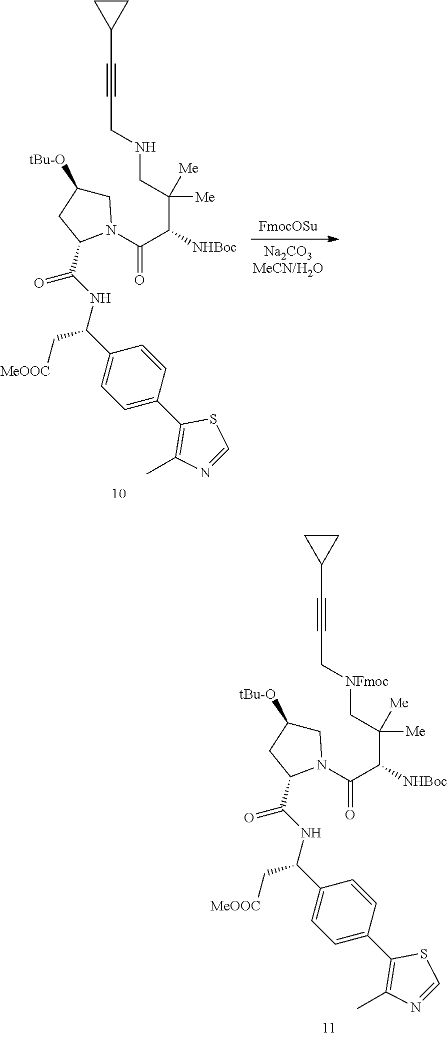

To a solution of 10 (1 equiv.) in MeCN/H₂O (2:1). Na₂CO₃ (3 equiv.) and FmocOSu (2.0 equiv.) were added. The reaction was stirred for 6 hours. The ethyl acetate was added and water phase was washed twice by ethyl acetate. The organic phase was combined, dried, concentrated by vacuum and purified by silica column chromatography to afford 10. UPLC-MS: calculated [M+H]⁺: 974.5, found: 974.6.

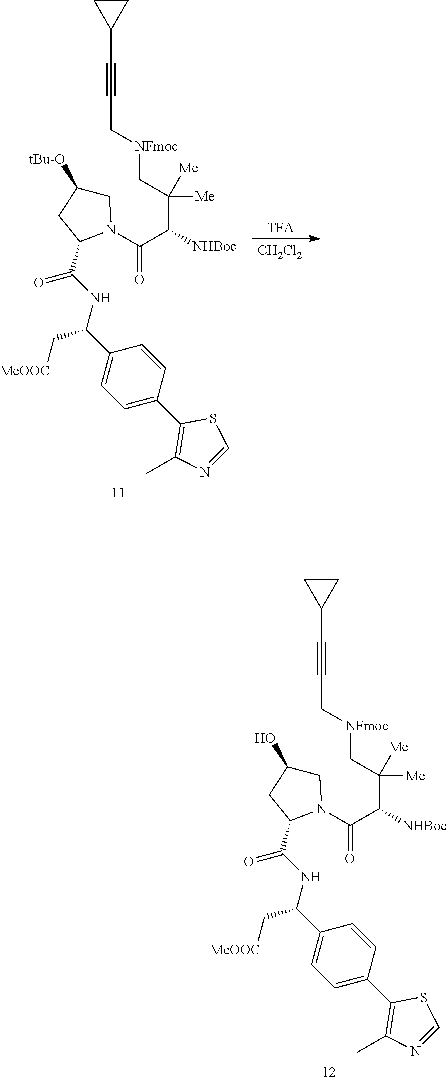

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 818.4, found: 818.2.

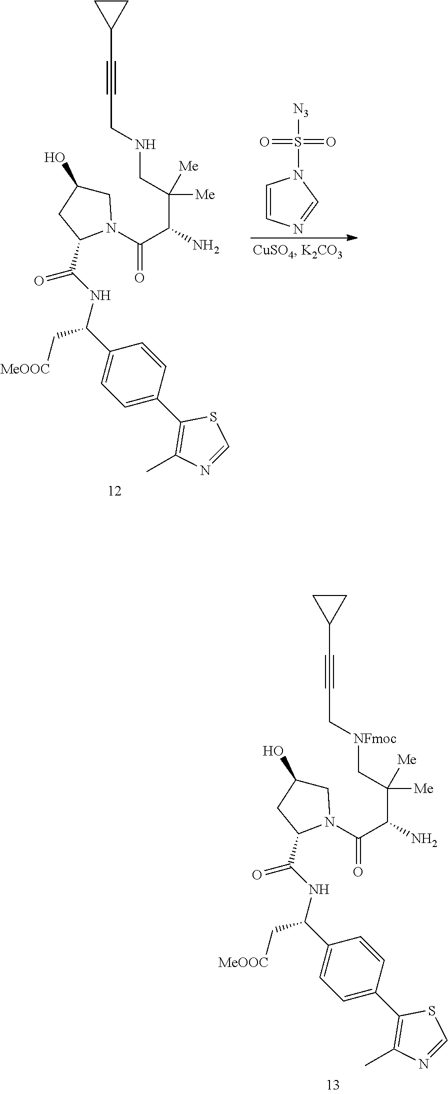

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 844.4, found: 844.5.

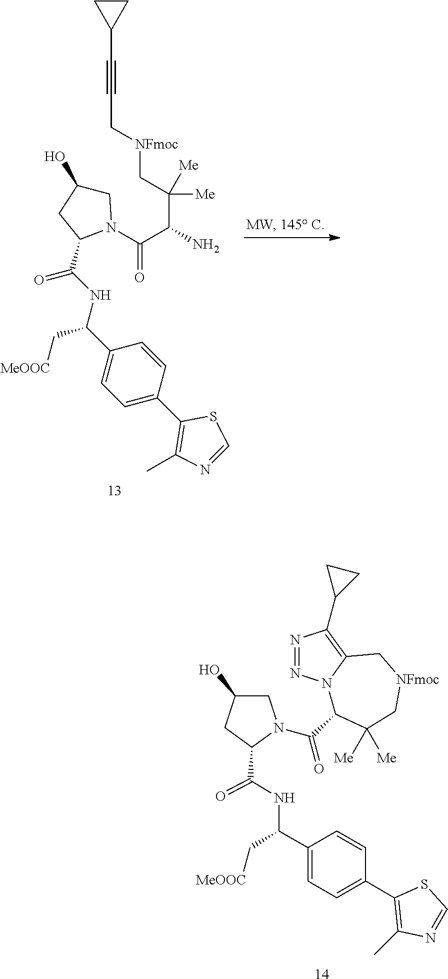

To a solution of 13 (1 equiv.) in 1,2-DCB (c₁₃=0.01 mol/L). Microwave the reaction at 145° C. for 16 h. The solvent was removed in vacuum for next step without further purification. UPLC-MS: calculated [M+H]⁺: 844.4, found: 844.4.

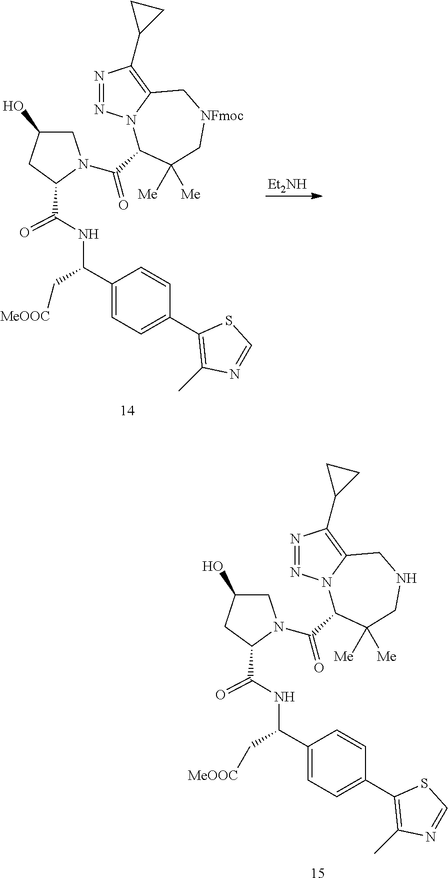

To a solution of 14 (1 equiv.) MeCN. Then diethylamine (5 equiv.) was added. The mixture was stirred for 3 hours. The mixture was detected by UPLC-MS and purified by HPLC to afford A10. UPLC-MS: calculated [M+H]⁺: 622.3, found: 622.5.

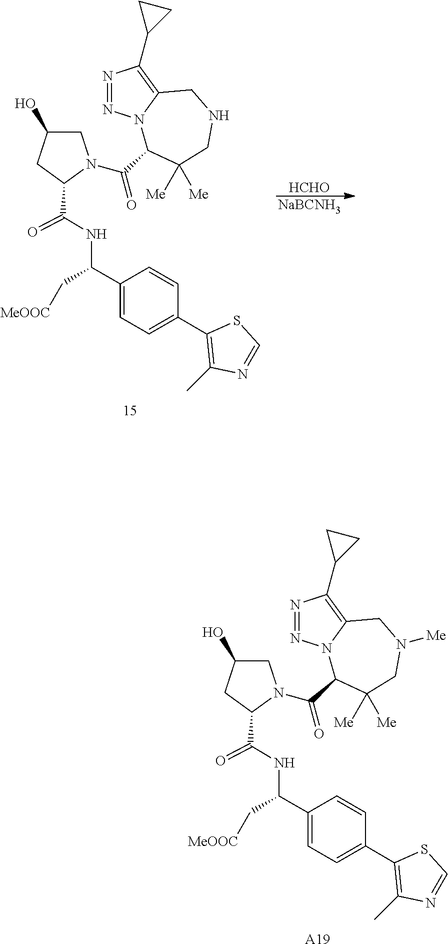 [SMILES: COC(=O)C[C@H](NC(=O)[C@@H]1C[C@@H](O)CN1C(=O)[C@@H]1n2nnc(C3CC3)c2CNCC1(C)C)c1ccc(-c2scnc2C)cc1 ; "NaBCNH3" ; "HCHO" ; "15" ; COC(=O)C[C@H](NC(=O)[C@@H]1C[C@@H](O)CN1C(=O)[C@H]1n2nnc(C3CC3)c2CN(C)CC1(C)C)c1ccc(-c2scnc2C)cc1 ; "A19"]

To a solution of 15 (1 equiv.) in MeOH (c₁₅=0.1 mol/L), HCHO aq. (37% formaldehyde solution, 13.4 mol/L, 5 equiv) was added. The mixture was stirred for 30 min. Then NaBH₃CN (3 equiv.) was added in 5 portions. The mixture was detected by UPLC-MS and purified by HPLC to afford A7. UPLC-MS: calculated [M+H]⁺: 636.3, found: 636.4.

### Compound A20. ethyl (R)-7-((2S,4R)-4-hydroxy-2-(((S)-1-(4-(4-methylthiazol-5-yl)phenyl)ethyl)carbamoyl)pyrrolidine-1-carbonyl)-6,6-dimethyl-4,5,6,7-tetrahydrobenzo[d]isoxazole-3-carboxylate

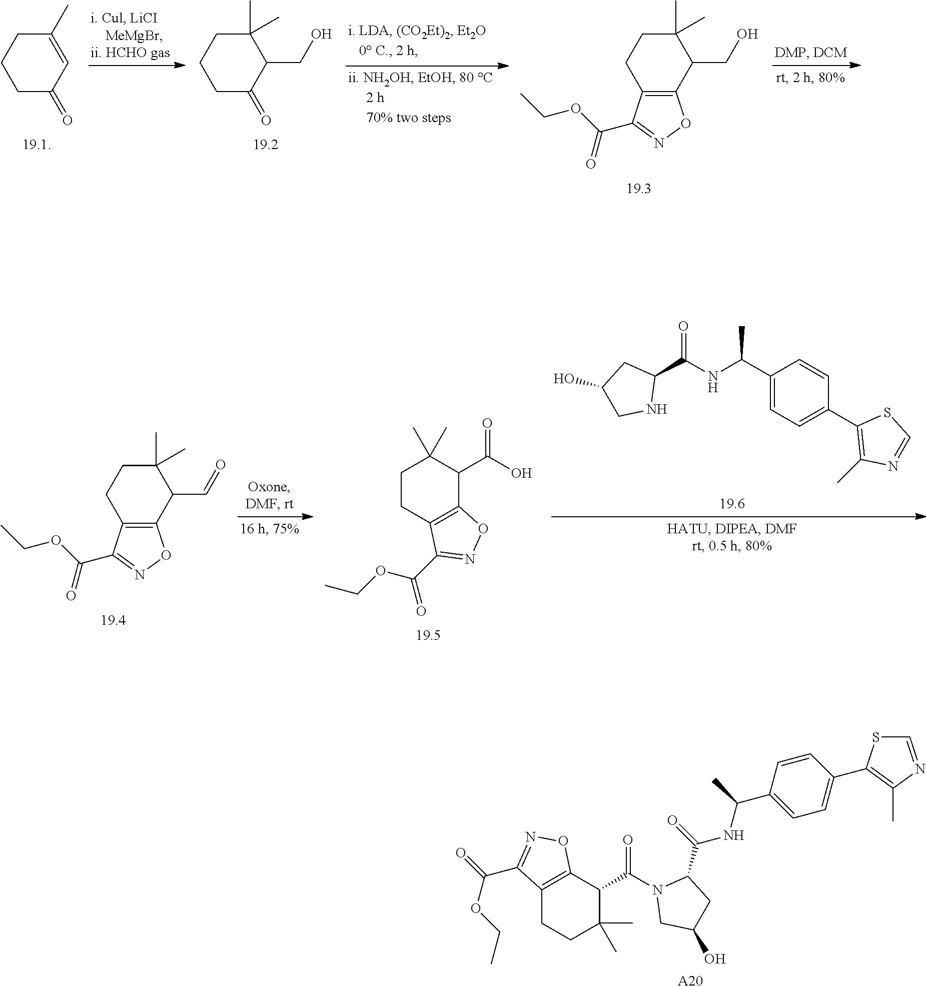

### Step 1

LiCl (0.1 eq) and CuI (0.05 eq) were added to a flame-dried two-way round bottom flask and purged with nitrogen three times. THF was added to the mixture and stirred vigorously until a homogeneous light-yellow solution. Compound 19.1 was added, and the reaction was cooled to 0° C. Methyl magnesium bromide (1.2 eq) was added to the reaction dropwise over 20 mins and the reaction was stirred at the same temperature for 2 h and monitored by TLC chromatography. After the complete conversion of starting material, the reaction was cooled to −10° C., and formaldehyde gas (generated by heating paraformaldehyde at 140° C.) was passed through the solution using a cannula for 4 h. The reaction was monitored by TLC chromatography. Upon completion of the reaction, the mixture was quenched with a saturated solution of ammonium chloride and extracted with ethyl acetate. The product was purified by flash column chromatography. The NMR (1H and 13C) matches with reported spectra.

### Step 2

A solution of compound 19.2 (1 eq), diethyl oxalate (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

### Step 3

A solution of crude product and hydroxyl amine hydrochloride (2 eq) in ethanol was heated to reflux for 2 h. After complete conversion of starting material, ethanol was evaporated under reduced pressure and the product was purified with flash column chromatography. UPLC-MS: calculated [M+H]⁺: 254.13, found: 254.12.

### Step 4

To a stirred solution of compound 19.3 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography. ¹H NMR (400 MHz, CDCl₃) δ 9.85 (d, J=2.4 Hz, 1H), 4.41 (q, J=7.1 Hz, 2H), 3.76 (dd, J=2.4, 1.4 Hz, 1H), 3.16 (dddd, J=16.0, 5.2, 3.1, 1.2 Hz, 1H), 2.60-2.47 (m, 1H), 1.89 (ddd, J=14.0, 11.5, 2.7 Hz, 1H), 1.83-1.63 (m, 3H), 1.40 (t, J=7.1 Hz, 3H), 1.27 (s, 3H), 0.98 (s, 3H). UPLC-MS: calculated [M+H]⁺: 252.13, found: 252.07.

### Step 5

To a stirred solution of compound 19.4 (1 eq) in DMF at rt, Oxone monohydrate (3 eq) was added. The reaction was stirred at rt for 16 h followed by dilution with water. The mixture was extracted with ethyl acetate and the product was purified using flash column chromatography. UPLC-MS: calculated [M+H]⁺: 268.13, found: 268.04.

### Step 6

To a stirred solution of compound 19.5 (1 eq), compound 19.6 (1 eq), and HATU (1 eq) in DMF at rt, DIPEA (1.5 eq) was added dropwise and monitored by UPLC. After completion of the reaction, the product was purified by reverse phase column chromatography to provide A20. UPLC-MS: calculated [M+H]⁺: 581.24, found: 581.10.

### Compound A21. (2R,4S)-1-((S)-3-(3-(2-chloro-3-hydroxyphenyl)propyl)-7,7-dimethyl-7,8-dihydro-4H,6H-[1,2,3]triazolo[5,1-c][1,4]oxazepine-8-carbonyl)-4-hydroxy-N—((R)-1-(4-(4-methylthiazol-5-yl)phenyl)ethyl)pyrrolidine-2-carboxamide

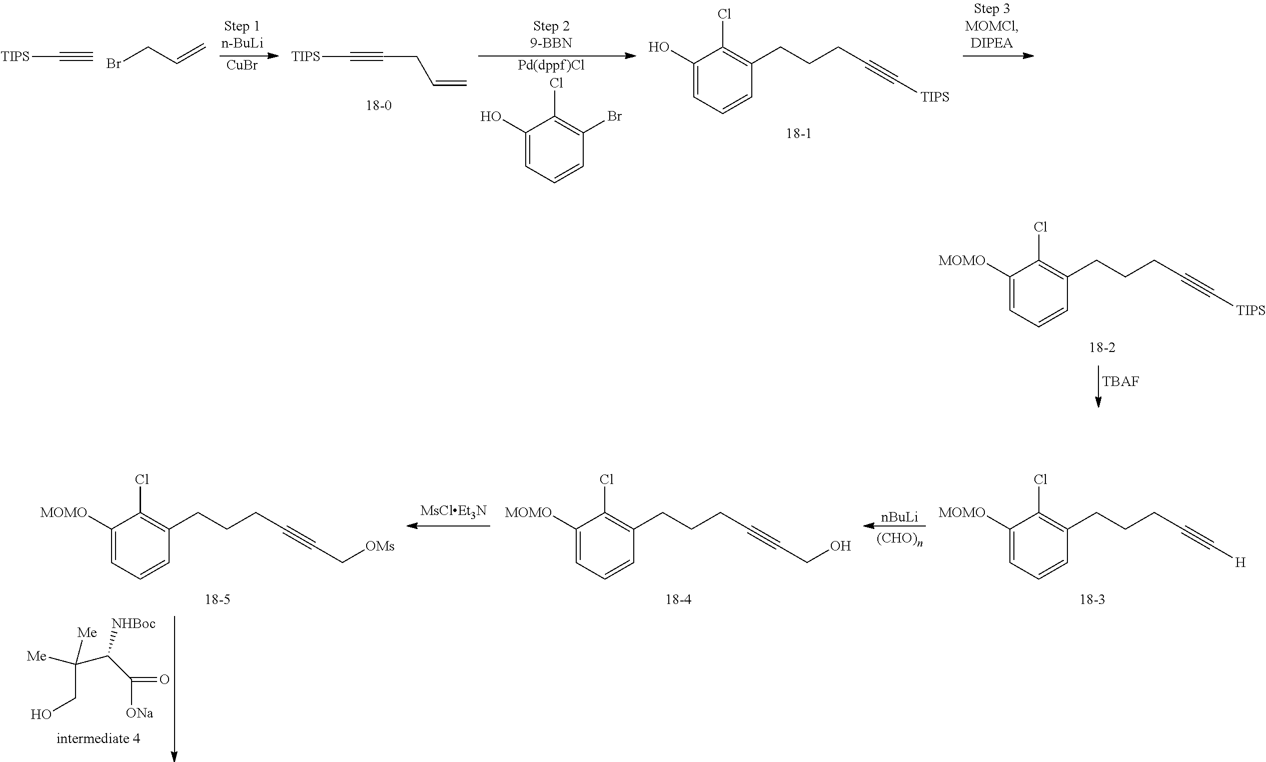

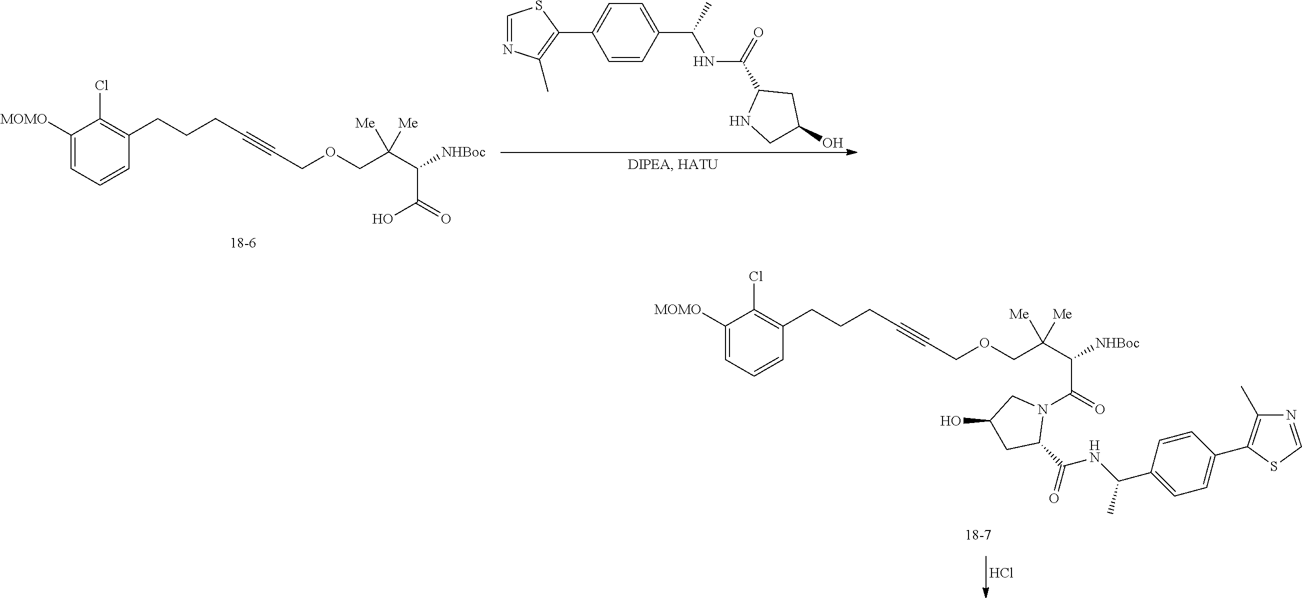

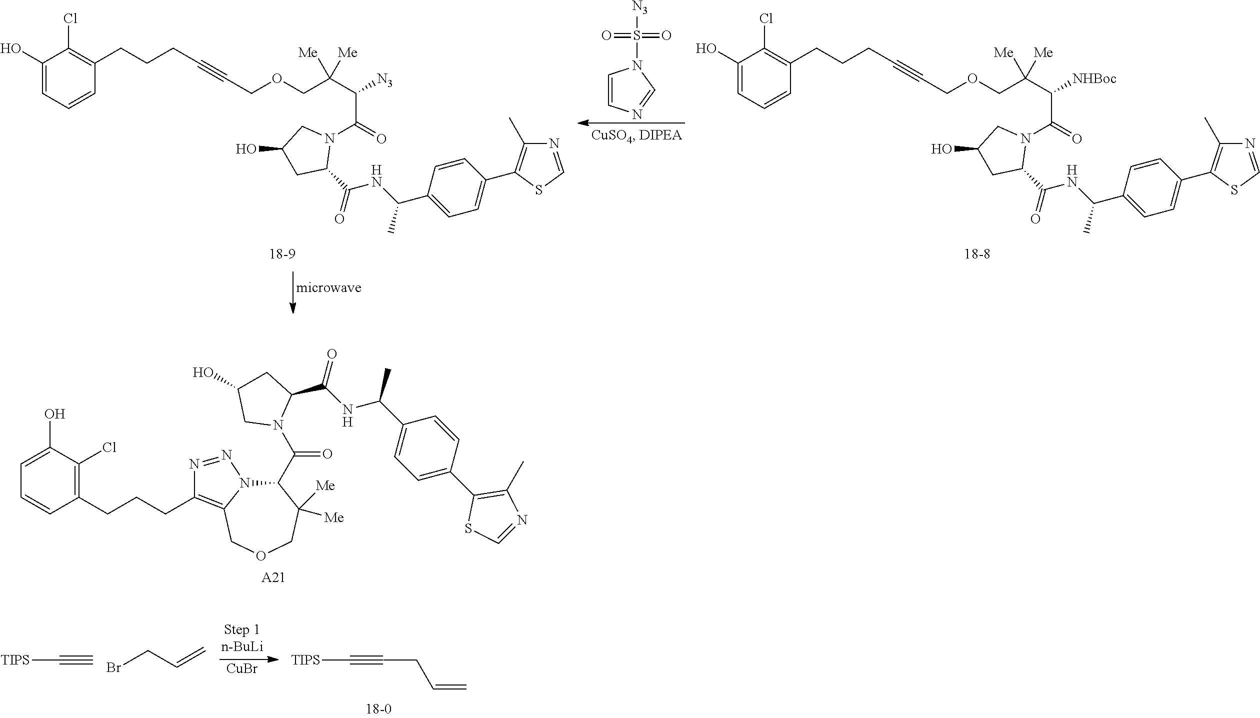

Step 1: n-BuLi in hexanes (17 ml, 2.5M, 1.1 equiv.) was added to a solution of (Triisopropylsilyl)acetylene (7 g, 1 equiv.) in THF (30 mL, c=1 mol/L) at −78° C. After stirring for 30 min at −78° C., CuBr (273 mg, 0.05 equiv.) and allyl bromide (3.6 mL, 1.1 equiv) were added. After stirring for 5 h at 50° C., the reaction mixture was quenched with saturated aqueous NH₄Cl, extracted with ether, washed with brine, dried over MgSO₄, and concentrated. Column chromatography (silica gel, hexanes) afforded 8.5 g 18-0 (98.5%) as a colorless oil. Rf₁₈₋₀=0.8, hexane. ¹H-NMR (400 MHz, CDCl₃) δ: 5.83 (ddt, 1H), 5.40 (dq, 1H), 5.13 (dq, 1H), 3.05 (dt, 2H), 1.07 (m, 21H)

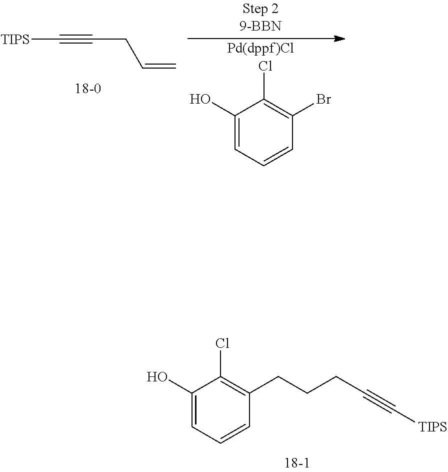

Step 2: 9-BBN in THF (218 mL, 0.05M 1.05 equiv.) was added to neat 18-0 (23 g, 1 equiv.) at 0° C. After stirring overnight at rt. NaOH aq (82 mL 4 equiv. C_{Na}OH=5 mol/L) was added and stir for 30 min at rt. 3-bromo-2-chlorophenol (25.6 g, 1.2 equiv.) and Pd(dppf)Cl₂ (4.4 g, 0.05 equiv.) were added and stir at 60° C. for 1 hour, the reaction mixture was quenched with saturated aqueous NaHCO₃, extracted with EA, washed with brine, dried over MgSO₄, and concentrated. Column chromatography (silica gel, hexanes/EA) afforded 27.8 g 18-1 (76.9%) as a pale-yellow oil. Rf₁₈₋₁=0.3, hexane/ethyl acetate=10:1. ¹H-NMR (400 MHz, CDCl₃) δ: 7.10 (t, 1H), 6.89 (dd, 1H), 6.82 (dd, 1H), 2.87 (t, 2H), 2.31 (t, 2H), 1.86 (m, 2H), 1.08 (m, 21H).

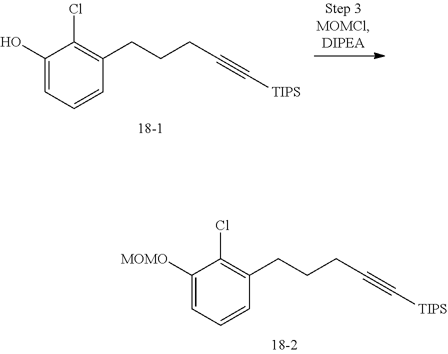

Step 3: To a solution of 18-1 (25 g, 1 equiv.) in 140 mL of dry dichloromethane, added DIPEA (38 mL, 3 equiv.) and MOMCl (6.5 mL, 1.2 equiv.). The mixture was stirred for 3 h. The organic layer was quenched with 50 mL of saturated NaHCO₃, Then the aqueous mixture was extracted twice with 100 mL of diethyl ether. The combined organic extract was washed once with brine (50 mL). The organic layer was dried over MgSO₄. Concentrated in vacuum and purified by silica gel flash chromatography (silica gel, hexanes/EA) to afford 27 g 18-2 (96%). Rf₁₈₋₂=0.7, hexane/ethyl acetate=10:1. ¹H-NMR (400 MHz, CDCl₃) δ: 7.14 (t, 1H), 7.06 (dd, 1H), 6.95 (dd, 1H), 5.27 (s, 2H), 3.55 (s, 3H), 2.93 (t, 2H), 2.34 (t, 2H), 1.87 (m, 2H), 1.11 (m, 21H).

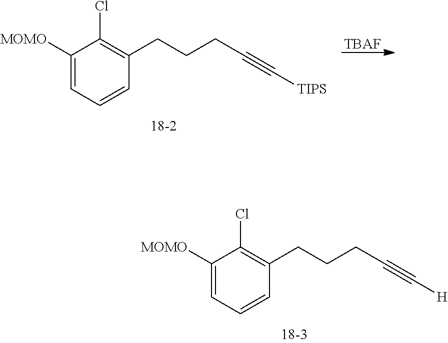

Step 4: TBAF (127 mL, 1M, 2 equiv.) was added to neat 18-2 (25 g, equiv.). The mixture was heated at 60° C. overnight. The reaction was quenched with 50 mL of saturated NH₄Cl. Then the aqueous mixture was extracted twice with 100 mL of ethyl acetate. The combined organic extract was washed once with brine (50 mL). The organic layer was dried over MgSO₄. Concentrated in vacuum and purified by silica gel flash chromatography (silica gel, hexanes/EA) to afford 14 g 18-3 (92.3%). Rf₁₈₋₃=0.7, hexane/ethyl acetate=10:1. ¹H-NMR (400 MHz, CDCl₃) δ: 7.12 (dd, 1H), 7.04 (dd, 1H), 6.92 (dd, 1H), 5.24 (s, 2H), 3.53 (s, 3H), 2.87 (t, 2H), 2.24 (t, 2H), 2.00 (t, 1H), 1.87 (m, 2H).

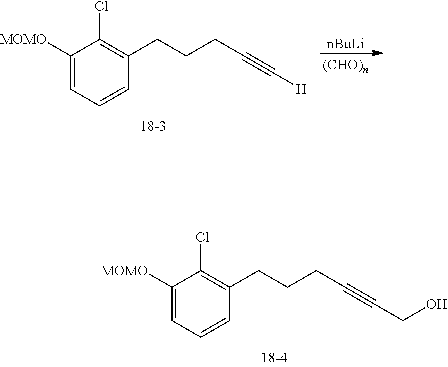

Step 5: To a solution of 18-3 (5.8 g, 1 equiv.) in 80 mL of dry THF. The mixture was stirred at −78° C., then n-BuLi (15 mL, 2.5M, 1.5 equiv.) was added. The mixture was stirred for 1 hour. Then (CHO)ₙ (1.8 g, 2.5 equiv) was added. The mixture was stirred overnight. The reaction was quenched with 50 mL of saturated NH₄Cl. Then the aqueous mixture was extracted twice with 50 mL of ethyl acetate. The combined organic extract was washed once with brine (50 mL). The organic layer was dried over MgSO₄. Concentrated in vacuum and purified by silica gel flash chromatography (silica gel, hexanes/EA) to afford 4 g 18-4 (61%). Rf₁₈₋₄=0.1, hexane/ethyl acetate=10:1. ¹H-NMR (400 MHz, CDCl₃) δ: 7.12 (dd, 1H), 7.04 (dd, 1H), 6.91 (dd, 1H), 5.24 (s, 2H), 4.27 (t, 2H), 3.53 (s, 3H), 2.85 (t, 2H), 2.27 (tt, 2H), 1.85 (m, 2H).

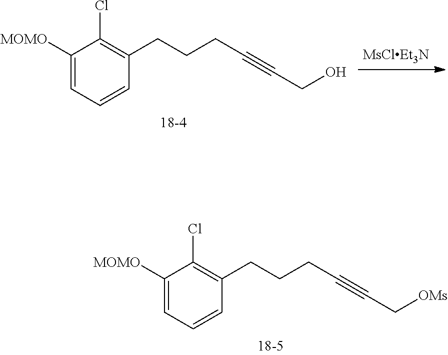

Step 6: To a solution of 18-4 (0.87 g, 1 equiv.) in dichloromethane. Et₃N (1.33 mL, 3 equiv.) was added. Then the mixture was stirred at 0° C. MsCl (0.38 mL, 1.5 equiv.) was added drop by drop into the mixture. The reaction was stirred for 30 min. Then reaction was quenched with 20 mL of saturated NaHCO₃. Then the aqueous mixture was extracted twice with 20 mL of ethyl acetate. The combined organic extract was washed once with brine (10 mL). The organic layer was dried over MgSO₄. Concentrated in vacuum to get 1 g 18-5 (88%) without further purification. Rf₁₈₋₅=0.6, dichloromethane/ethyl acetate=10:1. ¹H-NMR (400 MHz, CDCl₃): 7.16 (dd, 1H), 7.07 (dd, 1H), 6.92 (dd, 1H), 5.27 (s, 2H), 4.89 (t, 2H), 3.55 (s, 3H), 3.14 (s, 3H), 2.87 (t, 2H), 2.34 (tt, 2H), 1.89 (m, 2H).

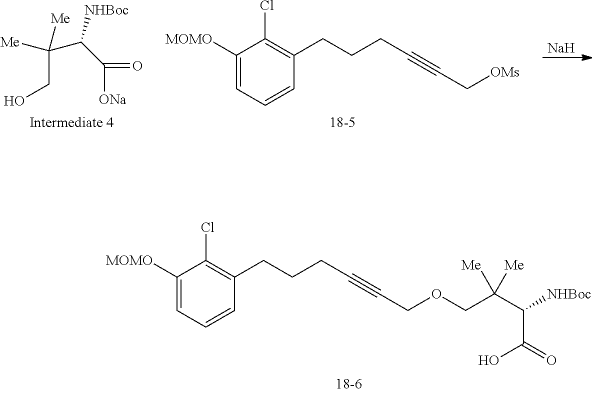

Step 7: To a solution of intermediate 4 (0.67 g, 1 equiv.) in DMF. NaH (150 mg, 60%, 1.5 equiv.) was added into mixture at 0° C. The mixture was stirred for 30 min. Then 18-5 (870 mg, 1 equiv.) was added. The mixture was stirred for 3 h at 0° C. Then reaction was quenched with 20 mL of water, the aqueous mixture was extracted fifth with 20 mL of ethyl acetate. The combined organic extract was washed once with brine (10 mL). The organic layer was dried over MgSO₄. Concentrated in vacuum to get crude 18-6 without further purification. UPLC-MS: calculated [M+Na]+: 520.21, found: 519.94.

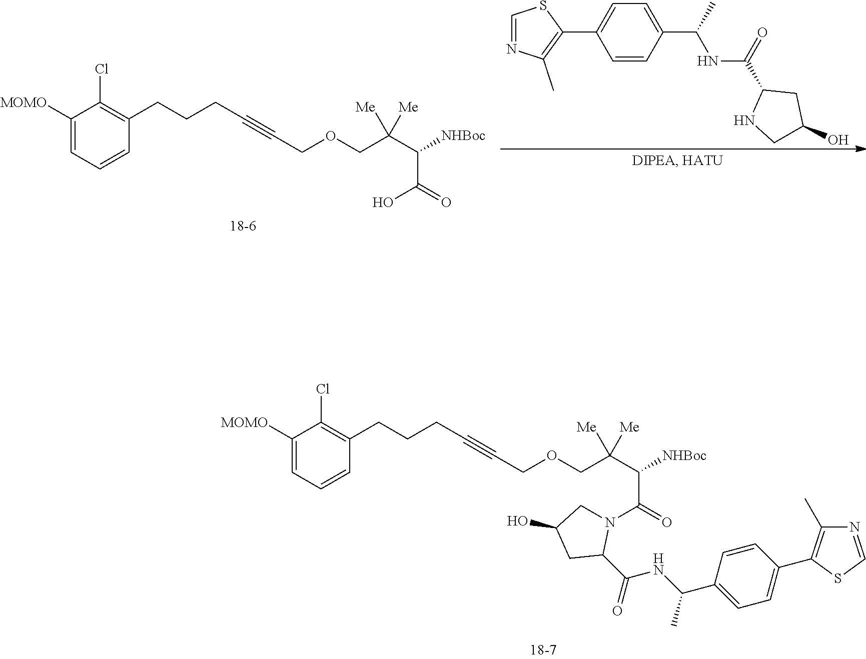

Step 8: To a solution of 18-6 in previous step in DMF. DIPEA (5 equiv.) and HATU (1 equiv.) was added into the mixture and stirred for 10 min. Then (2S,4R)-4-hydroxy-N—((S)-1-(4-(4-methylthiazol-5-yl)phenyl)ethyl)pyrrolidine-2-carboxamide (1 equiv.) was added. The mixture was stirred for 10 min. Then purified by HPLC directly. UPLC-MS: calculated [M+H]+: 811.35, found: 810.85.

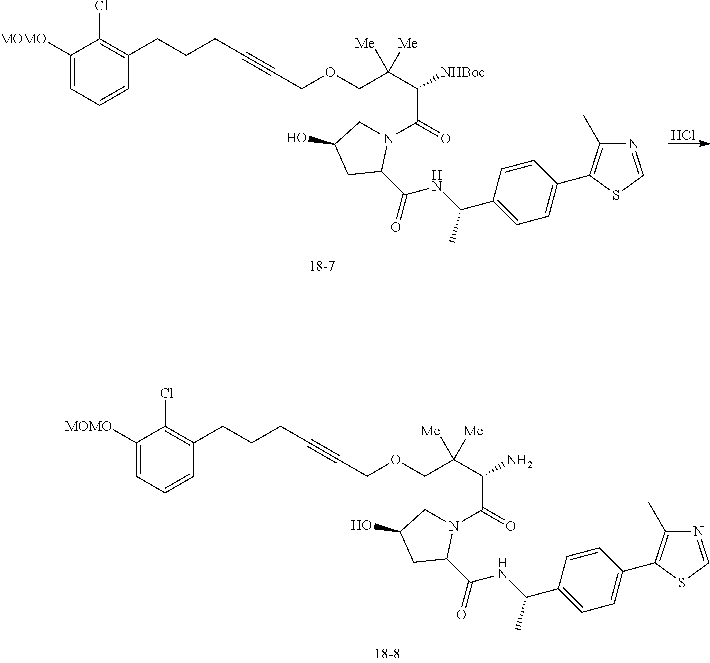

Step 8: To a solution of 18-7 in MeCN (c=0.5M), hydrochloric acid (12M, same value with MeCN) was added. The mixture was stirred for 1 h, then removed all solvent in vacuum to afford 18-8. UPLC-MS: calculated [M+H]+: 667.27, found: 666.6.

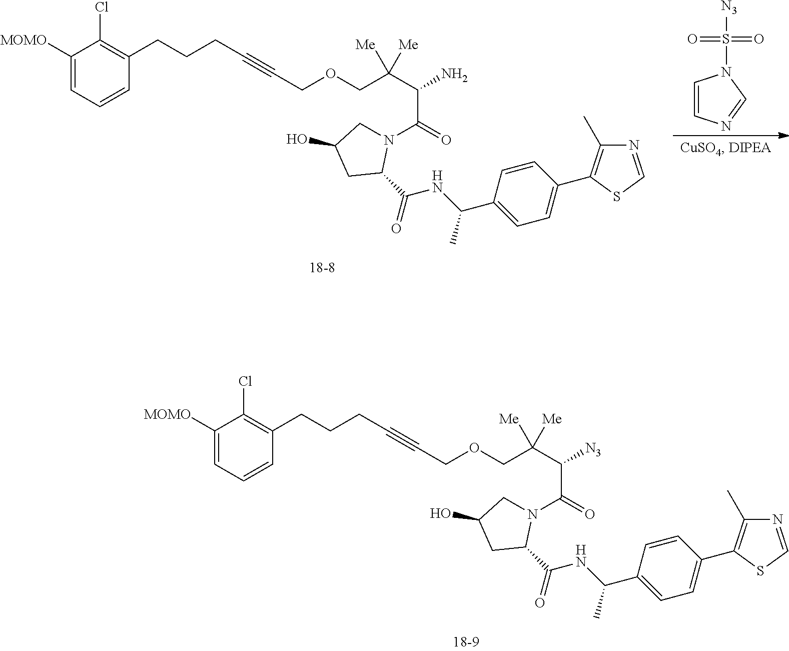

Step 9: To a solution of 18-8 (400 mg, 1 equiv.) in MeOH. Then DIPEA (0.42 mL, 4 equiv.) was added. The mixture was stirred for 5 min. Then 1H-imidazole-1-sulfonyl azide (250 mg, 2 equiv.) and CuSO₄ (25 mg, 0.2 equiv.) was added at room temperature. The mixture was stirred for 3 h, then directly purified by HPLC to afford 320 mg 18-9 (77%). UPLC-MS: calculated [M+H]+: 693.26, found: 692.84.

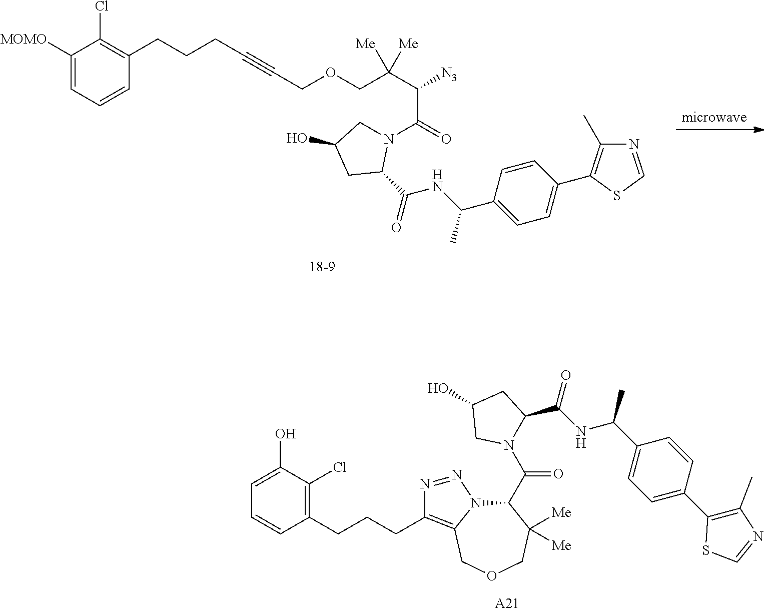

Step 10: To a solution of 18-9 (1 equiv.) in 1,2-DCB (c₁₃=0.01 mol/L). Microwave the reaction at 140° C. for 16 h. The solvent was removed in vacuum and purified by HPLC to afford A21. UPLC-MS: calculated [M+H]+: 693.26, found: 692.73.

### Compound A22. (2S,4R)-4-hydroxy-N—((S)-1-(4-(4-methylthiazol-5-yl)phenyl)ethyl)-1-((R)-3,7,7-trimethyl-5,6,7,8-tetrahydro-4H-cyclohepta[d]isoxazole-8-carbonyl)pyrrolidine-2-carboxamide

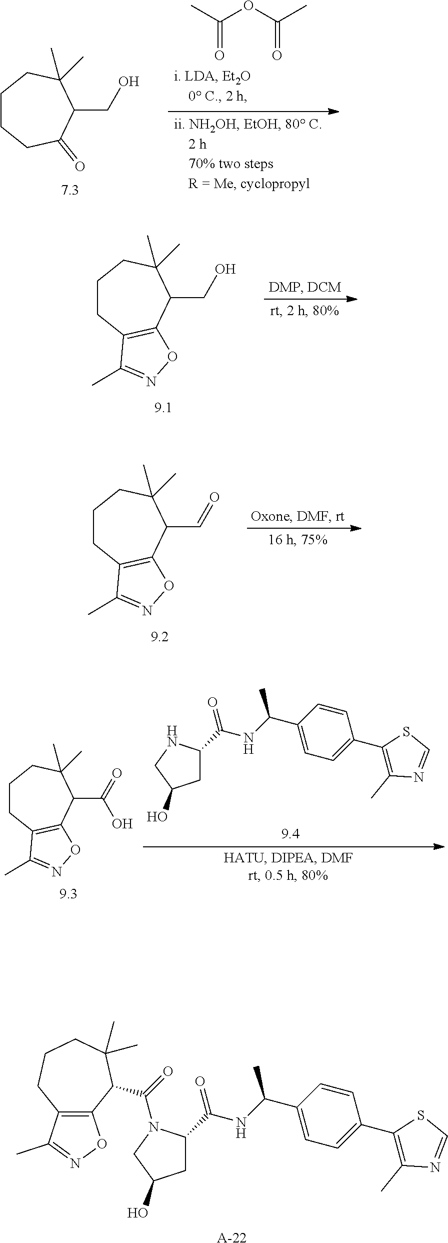

Step 1. A solution of compound 7.3 (1 eq), acetic anhydride (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step. A solution of crude product and hydroxyl amine hydrochloride (2 eq) in ethanol was heated to reflux for 2 h. After complete conversion of starting material, ethanol was evaporated under reduced pressure and the product was purified with flash column chromatography to get compound 9.1 (UPLC-MS: calculated [M+H]⁺: 210.15, found: 210.17).

Step 2: To a stirred solution of compound 9.1 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography to get compound 9.2 (UPLC-MS: calculated [M+H]⁺: 208.14, found: 208.17).

Step 3. To a stirred solution of compound 9.2 (1 eq) in DMF at rt, Oxone monohydrate (3 eq) was added. The reaction was stirred at rt for 16 h followed by dilution with water. The mixture was extracted with ethyl acetate and the product was purified using flash column chromatography. 9.3 (UPLC-MS: calculated [M+H]⁺: 224.12, found: 224.16).

Step 4: To a stirred solution of compound 9.3 (1 eq), compound 9.4 (1 eq), and HATU (1 eq) in DMF at rt, DIPEA (1.5 eq) was added dropwise and monitored by UPLC. After completion of the reaction, the product was purified by reverse phase column chromatography to get compound A-22 as white solid, (40% yields, 1:2.5 dr) UPLC-MS: calculated [M+H]⁺: 537.25, found: 537.25.

Compounds A23. methyl (S)-3-((2S,4R)-4-hydroxy-1-((R)-3,7,7-trimethyl-5,6,7,8-tetrahydro-4H-cyclohepta[d]isoxazole-8-carbonyl)pyrrolidine-2-carboxamido)-3-(4-(4-methylthiazol-5-yl)phenyl)propanoate

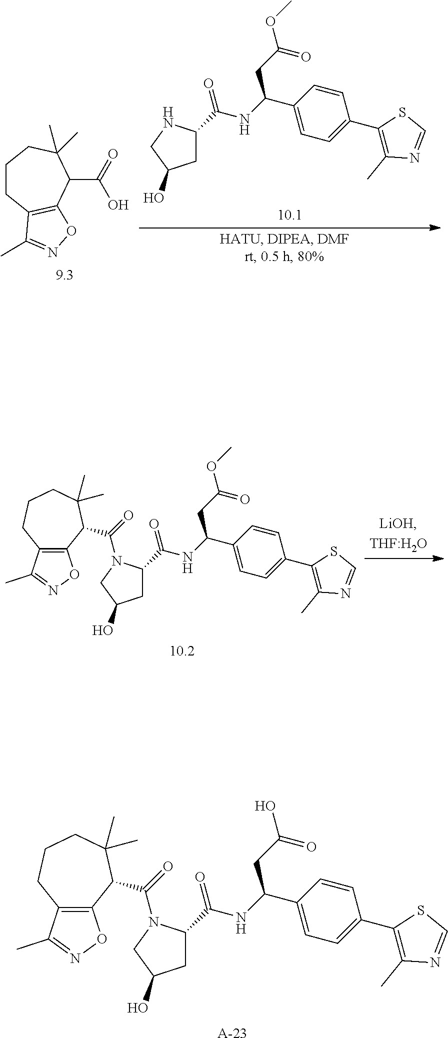

Step 1: To a stirred solution of compound 9.3 (1 eq), compound 9.4 (1 eq), and HATU (1 eq) in DMF at rt, DIPEA (1.5 eq) was added dropwise and monitored by UPLC. After completion of the reaction, the product was purified by reverse phase column chromatography to get compound 10.2 as white solid, (41% yields, 1:2.5 dr) UPLC-MS: calculated [M+H]⁺: 595.25, found: 595.30.

Step 2: To a stirred solution of compound 9.5 in THF:water (1:1) at rt, LiOH was added and the reaction was stirred for 2 h. After complete conversion of the starting material, it was acidified with 2 N HCl up to pH 3 and extracted with ethyl acetate. The product was purified with flash column chromatography to get compound A-23 UPLC-MS: calculated [M+H]⁺: 581.24, found: 581.32.

### Compounds A24. methyl (S)-3-((2S,4R)-1-((R)-3-cyclopropyl-7,7-dimethyl-5,6,7,8-tetrahydro-4H-cyclohepta[d]isoxazole-8-carbonyl)-4-hydroxypyrrolidine-2-carboxamido)-3-(4-(4-methylthiazol-5-yl)phenyl)propanoate

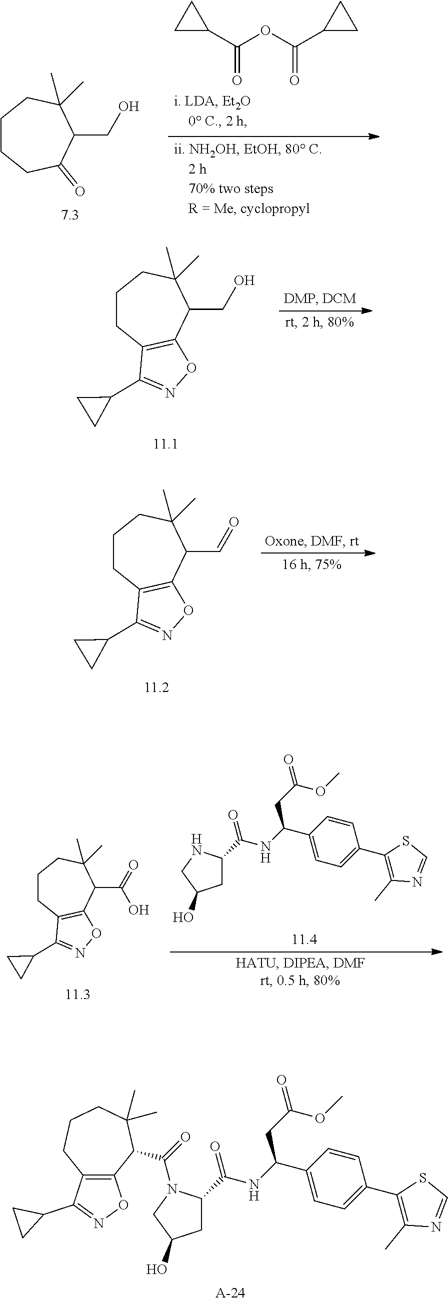

Step 1. A solution of compound 7.3 (1 eq), cyclopropanecarboxylic anhydride (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step. A solution of crude product and hydroxyl amine hydrochloride (2 eq) in ethanol was heated to reflux for 2 h. After complete conversion of starting material, ethanol was evaporated under reduced pressure and the product was purified with flash column chromatography to get compound 11.1 (UPLC-MS: calculated [M+H]⁺: 236.16, found: 236.19).

Step 2: To a stirred solution of compound 11.1 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography to get compound 11.2 (UPLC-MS: calculated [M+H]⁺: 234.14, found: 234.12).

Step 3. To a stirred solution of compound 11.2 (1 eq) in DMF at rt, Oxone monohydrate (3 eq) was added. The reaction was stirred at rt for 16 h followed by dilution with water. The mixture was extracted with ethyl acetate and the product was purified using flash column chromatography. 11.3 (UPLC-MS: calculated [M+H]⁺: 250.14, found: 250.14).

Step 4: To a stirred solution of compound 11.3 (1 eq), compound 11.4 (1 eq), and HATU (1 eq) in DMF at rt, DIPEA (1.5 eq) was added dropwise and monitored by UPLC. After completion of the reaction, the product was purified by reverse phase column chromatography to get compound A-24 as white solid, (42% yields, 1:2.5 dr) UPLC-MS: calculated [M+H]⁺: 537.25, found: 537.25.

### Compounds A25. (2S,4R)—N—((R)-2-azido-1-(4-(4-methylthiazol-5-yl)phenyl)ethyl)-1-((R)-3-cyclopropyl-7,7-dimethyl-5,6,7,8-tetrahydro-4H-cyclohepta[d]isoxazole-8-carbonyl)-4-hydroxypyrrolidine-2-carboxamide

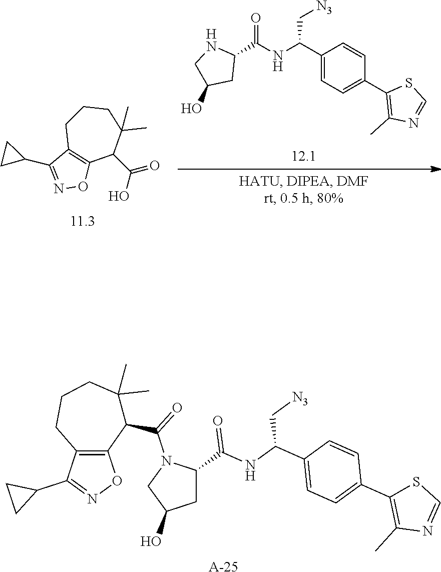 [SMILES: CC1(C)CCCc2c(C3CC3)noc2C1C(=O)O ; Cc1ncsc1-c1ccc([C@@H](CN=[N+]=[N-])NC(=O)[C@@H]2C[C@@H](O)CN2)cc1 ; Cc1ncsc1-c1ccc([C@@H](CN=[N+]=[N-])NC(=O)[C@@H]2C[C@@H](O)CN2C(=O)[C@H]2c3onc(C4CC4)c3CCCC2(C)C)cc1 ; "A-25"]

To a stirred solution of compound 11.3 (1 eq), compound 12.1 (1 eq), and HATU (1 eq) in DMF at rt, DIPEA (1.5 eq) was added dropwise and monitored by UPLC. After completion of the reaction, the product was purified by reverse phase column chromatography to get compound A-24 as white solid, (35% yields, 1:2.5 dr) UPLC-MS: calculated [M+H]⁺: 604.26, found: 604.32.

### Compounds A26. methyl (S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(2,4-dimethyloxazol-5-yl)phenyl)propanoate

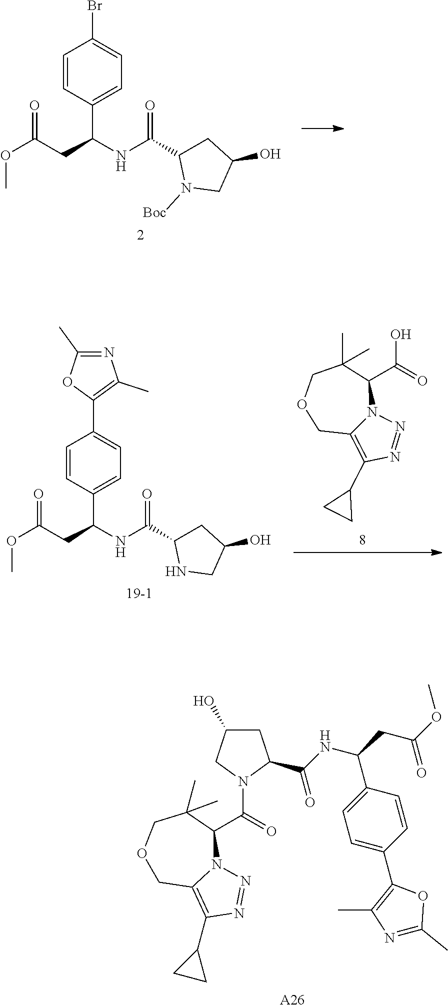

19-1: Pd(OAc)₂ (0.1 mmol, 0.11 equiv.) was added to a mixture of compound 2 (1.0 mmol, 1 equiv.), 2,4-dimethyloxazole (2 mmol, 2 equiv.) and Potassium acetate (2 mmol, 2 equiv) in DMF (5 mL). The resulting mixture was purged and refilled with argon three times and stirred at 100° C. overnight under Argon. The reaction mixture was then cooled to room temperature and evaporated to remove most of the solvent. The residue was purified by HPLC and the obtained product was treated with 5 mL of 1M HCl (in Methanol) and 2 mL of 4M HCl (in dioxane) for 2 h. The reaction mixture was evaporated to give the 19-1 without further purification. UPLC-MS calculated [M+H]⁺: 388.2, found: 388.5. Retention time: 1.8 min. A26: HATU (0.022 mmol, 1.1 equiv.) was added to a solution of 19-1 (0.02 mmol, 1 equiv.), 8 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield A26. UPLC-MS calculated [M+H]²+: 621.3, found: 621.5. Retention time: 3.7 min.

TABLE E1
Mass Spec Data

| Compound No. | Expected [M + H]⁺ | Observed [M + H]⁺ |
|---|---|---|
| A1 | 537.2 | 537.7 |
| A2 | 565.3 | 565.6 |
| A3 | 623.3 | 623.5 |
| A4 | 524.2 | 524.4 |
| A5 | 495.2 | 495.0 |
| A6 | 524.2 | 524.6 |
| A7 | 538.3 | 538.4 |
| A8 | 566.3 | 566.8 |
| A9 | 654.3 | 654.1 |
| A10 | 564.3 | 564.3 |
| A11 | 579.2 | 579.4 |
| A12 | 664.3 | 664.1 |
| A13 | 609.24 | 608.95 |
| A14 | 672.29 | 671.94 |
| A15 | 638.3 | 638.3 |
| A16 | 595.25 | 595.10 |
| A17 | 652.3 | 652.2 |
| A18 | 637.3 | 637.6 |
| A19 | 636.3 | 636.4 |
| A20 | 581.24 | 581.10 |
| A21 | 693.26 | 692.73 |
| A22 | 537.25 | 537.25 |
| A23 | 581.24 | 581.32 |
| A24 | 621.27 | 621.26 |
| A25 | 604.26 | 604.30 |
| A26 | 621.3 | 621.5 |

### B. Bifunctional Degraders

### Compound B1. (2-(((5S,8S,10aR)-3-acetyl-8-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

### Synthesis of Intermediate 11

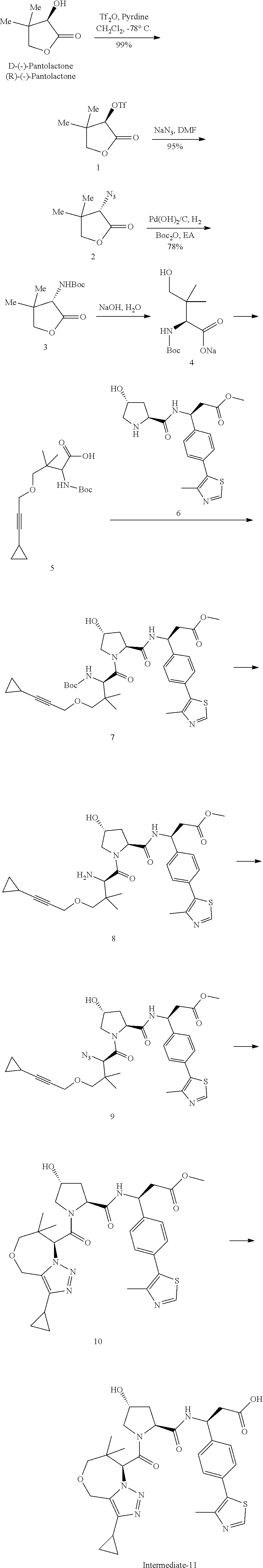

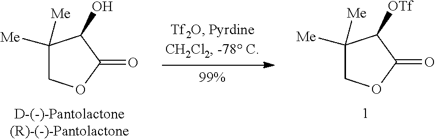 [SMILES: CC1(C)COC(=O)[C@@H]1O ; CC1(C)COC(=O)[C@@H]1OS(=O)(=O)C(F)(F)F]

To a solution of (R)-(−)-Pantolactone (10 g, 1 equiv.) in 4 mL of dry dichloromethane was added pyridine (7.8 mL, 1.25 equiv.). The resulting solution was cooled to −78° C. Triflic anhydride (13.9 mL, 1.1 equiv.) was added dropwise via a syringe over 10 min. The reaction mixture was stirred at −78° C. for 30 min and then 1 h at room temperature. The reaction mixture was partitioned between 200 mL of diethyl ether and 100 mL of 20% HCl. The organic layer was washed once with 50 mL of saturated NaHCO₃, followed by 50 mL of brine. It was dried over MgSO₄ and concentrated in vacuum to afford 20 g 1 (99%), which was used in subsequent step without further purification. Rf₁=0.5, hexane/ethyl acetate=7:3.

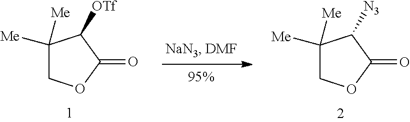 [SMILES: CC1(C)COC(=O)[C@@H]1OS(=O)(=O)C(F)(F)F ; CC1(C)COC(=O)[C@H]1N=[N+]=[N-]]

The crude product 1 (20 g, 1 equiv.) was dissolved in 50 mL of DMF. To the resulting solution was added NaN₃ (5.7 g, 1.15 equiv.). The mixture was stirred at room temperature for 2 h. The reaction mixture was poured into water (200 mL). The aqueous mixture was extracted twice with 100 mL of diethyl ether. The combined organic extract was washed once with brine (50 mL). The organic layer was dried over MgSO₄. Concentrated in vacuum and purified by silica gel flash chromatography to afford 11.3 g white powder 2 (95%). Rf₂=0.26, hexane/ethyl acetate=8:2

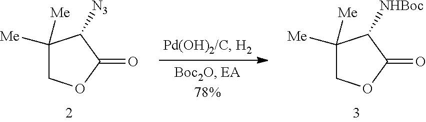 [SMILES: CC1(C)COC(=O)[C@H]1N=[N+]=[N-] ; CC(C)(C)OC(=O)N[C@@H]1C(=O)OCC1(C)C]

To a solution of 2 (2 g, 1 equiv.) in ethyl acetate (70 mL). Boc₂O (5.6 g, 2 equiv.) and Pd(OH)₂/C (0.2 g) were added into the flask. Hydrogenation with H₂ balloon for 24 h. Rf₂=0.71, Rf₃=0.43 Dichloromethane. Pd(OH)₂/C was filtered by Cilite. Concentrated in vacuum and purified by silica gel flash chromatography to afford 2.5 g white powder 3 (84.6%). UPLC-MS: calculated [M+Na]⁺: 252.12, found: 252.13.

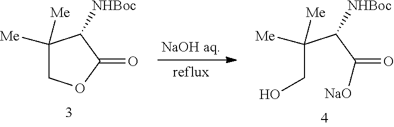

To a solution of 3 (150 mg, 1 equiv.) in NaOH aq. (5.2 mL, C_{NaOH}=0.125 mol/L, 1 equiv). The mixture was stirred at 40° C. for two hours. Once the reaction was completed detected by HPLC-MS, remove the water by freeze drying in vacuum to afford 4, which was used in subsequent step without further purification. UPLC-MS: calculated [M+H]⁺: 248.15, found: 247.99.

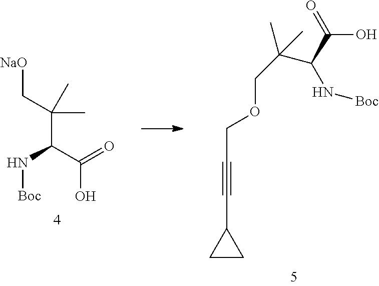

Sodium (S)-3-((tert-butoxycarbonyl)amino)-3-carboxy-2,2-dimethylpropan-1-olate (4, 100 mg, 0.37 mmol, 1 equiv.) was dissolved in DMF (3 ml). The solution was cooled to 0° C. and NaH (23 mg, 0.56 mmol, 60 wt-% suspension in mineral oil, 1.5 equiv) was added in portions. After stirring for 0.5-1 h at 0° C., (3-bromoprop-1-yn-1-yl)cyclopropane was added dropwise.

The reaction was slowly (1-2 h) warmed to rt and stirred at rt for 2-4 h. The reaction was quenched by addition of 1 mL water, and then acidified with 1M KHSO4 to pH 1-2. The resulting mixture was extracted with EtOAc and the combined organic phases were washed with brine. The organic layer was dried over Na₂SO₄, filtered and concentrated in vacuo to afford crude 5 which can be purified by HPLC to give 5. UPLC-MS calculated [M+H]⁺: 326.2, found: 270.3, 248.4, 174.2. Retention time: 4.6 min.

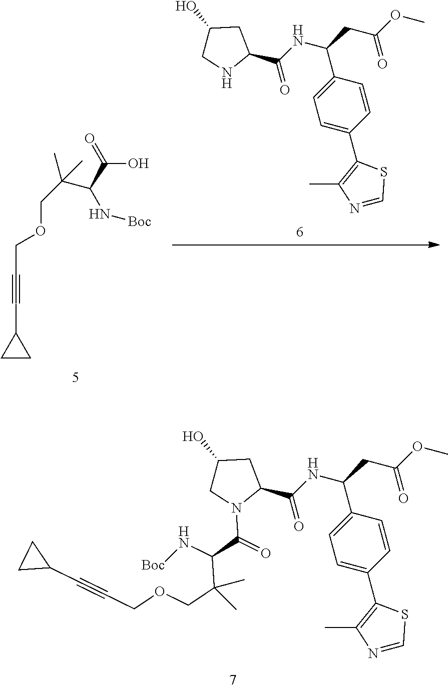 [SMILES: CC(C)(C)OC(=O)N[C@H](C(=O)O)C(C)(C)COCC#CC1CC1 ; COC(=O)C[C@H](NC(=O)[C@@H]1C[C@@H](O)CN1)c1ccc(-c2scnc2C)cc1 ; COC(=O)C[C@H](NC(=O)[C@@H]1C[C@@H](O)CN1C(=O)[C@H](NC(=O)OC(C)(C)C)C(C)(C)COCC#CC1CC1)c1ccc(-c2scnc2C)cc1]

HATU (0.18 mmol, 1.1 equiv.) was added to a solution of 5 (0.16 mmol, 1 equiv.), 6 (0.16 mmol, 1 equiv.) and DIEA (0.48 mmol, 3 equiv.) in DMF (2 mL) and the resulting mixture was stirred at rt for 1 h. This mixture was purified by reverse phase preparative HPLC to give 7. UPLC-MS calculated [M+H]⁺: 697.3, found: 697.5. Retention time: 5.2 min.

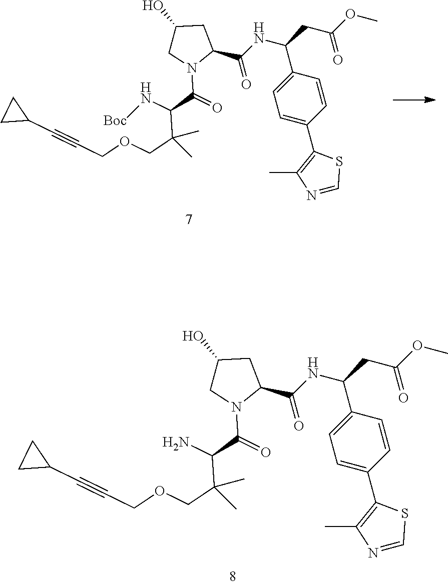 [SMILES: COC(=O)C[C@H](NC(=O)[C@@H]1C[C@@H](O)CN1C(=O)[C@H](NC(=O)OC(C)(C)C)C(C)(C)COCC#CC1CC1)c1ccc(-c2scnc2C)cc1 ; COC(=O)C[C@H](NC(=O)[C@@H]1C[C@@H](O)CN1C(=O)[C@H](N)C(C)(C)COCC#CC1CC1)c1ccc(-c2scnc2C)cc1]

10% H2SO₄ was added to a solution of compound 7 (0.05 mmol) in MeCN (0.5 mL). and the resulting reaction solution was stirred at rt for 4 h. This mixture was purified by reverse phase preparative HPLC to give 8. UPLC-MS calculated [M+H]⁺: 597.3, found: 597.8. Retention time: 3.3 min.

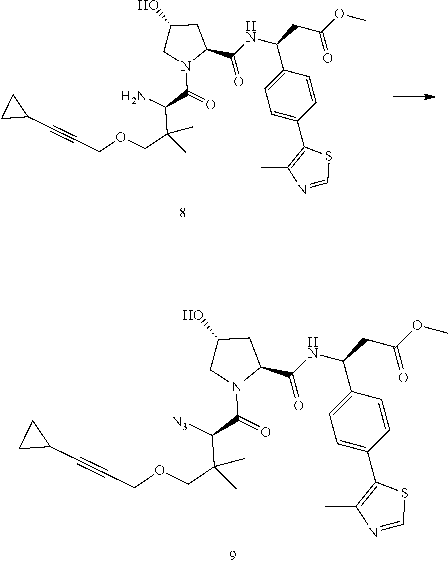 [SMILES: COC(=O)C[C@H](NC(=O)[C@@H]1C[C@@H](O)CN1C(=O)[C@H](N)C(C)(C)COCC#CC1CC1)c1ccc(-c2scnc2C)cc1 ; COC(=O)C[C@H](NC(=O)[C@@H]1C[C@@H](O)CN1C(=O)[C@H](N=[N+]=[N-])C(C)(C)COCC#CC1CC1)c1ccc(-c2scnc2C)cc1]

1H-imidazole-1-sulfonyl azide (0.12 mmol) was added to a mixture of 8 (0.1 mmol), potassium carbonate (0.25 mmol) and cupric sulfate (0.02 mmol) in methanol (1 mL) at 25° C. The reaction mixture was stirred at 25° C. for 10 h. After removing the solvent, 10% potassium bisulfate in water (1 mL) was added and extracted with ethyl acetate. The organic layer was washed with brine, and dried over sodium sulfate. The organic layer was concentrated, and the residue was purified by reverse phase preparative HPLC to give 9. UPLC-MS calculated [M+H]⁺: 623.3, found: 623.5. Retention time: 4.8 min.

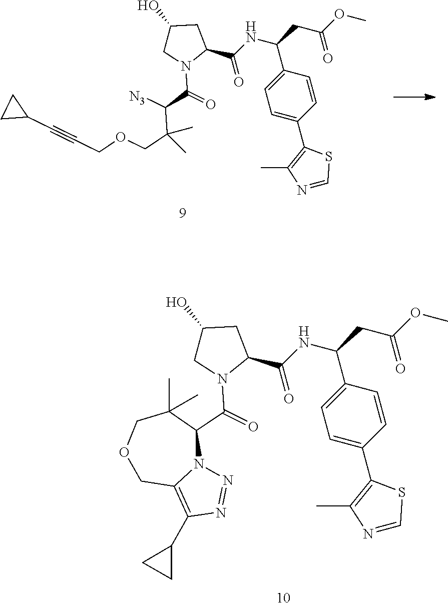 [SMILES: COC(=O)C[C@H](NC(=O)[C@@H]1C[C@@H](O)CN1C(=O)[C@H](N=[N+]=[N-])C(C)(C)COCC#CC1CC1)c1ccc(-c2scnc2C)cc1 ; COC(=O)C[C@H](NC(=O)[C@@H]1C[C@@H](O)CN1C(=O)[C@H]1n2nnc(C3CC3)c2COCC1(C)C)c1ccc(-c2scnc2C)cc1]

To a solution of 9 (30 mg) in 1,2-dichlorobenzene (1 mL). Microwave the reaction at 140° C. for 16 h. Removal of the solvent under vacuum to yield the crude product 10 which was used for the next step without further purification. UPLC-MS calculated [M+H]⁺: 622.3, found: 622.5. Retention time: 3.2 min.

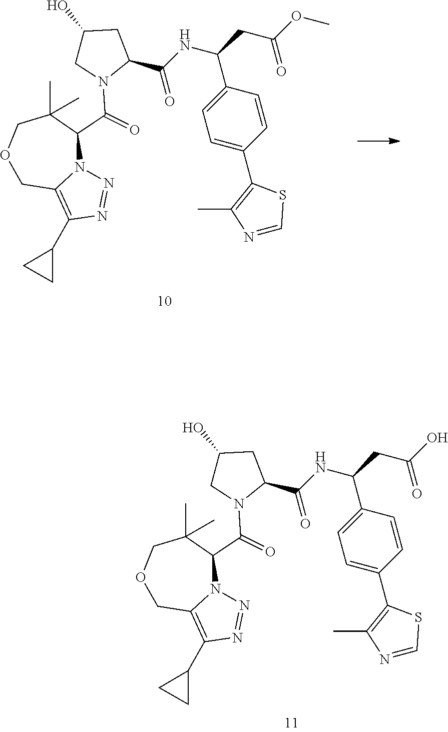 [SMILES: COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; Cc1ncsc1-c1ccc([C@H](CC(=O)O)NC(=O)[C@@H]2C[C@@H](O)CN2C(=O)[C@H]2n3nnc(C4CC4)c3COCC2(C)C)cc1]

LiOH (0.2 mmol in 1 mL H2O) was added to a solution of 10 (0.1 mmoL) in Methanol (1 ml) and the resulting mixture was stirred for 2 h. Acidified with 1M HCl and then purification was performed by HPLC to give 11. UPLC-MS calculated [M+H]⁺: 609.2, found: 609.4. Retention time: 2.6 min.

Scheme 2 for synthesis of Intermediate 11

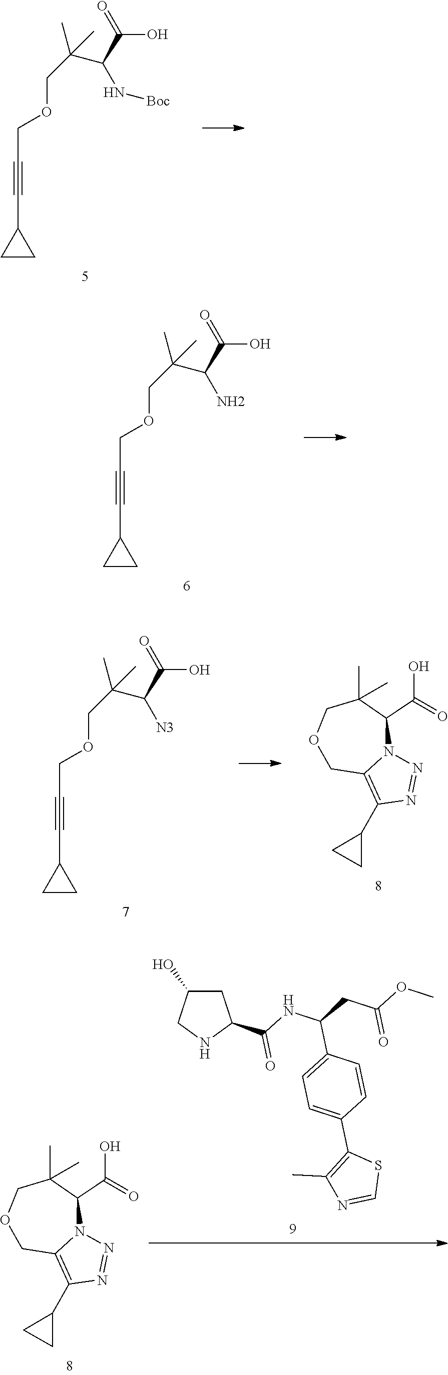 [SMILES: CC(C)(C)OC(=O)N[C@H](C(=O)O)C(C)(C)COCC#CC1CC1 ; CC(C)(COCC#CC1CC1)[C@H](N)C(=O)O ; CC(C)(COCC#CC1CC1)[C@H](N=[N+]=[N-])C(=O)O ; CC1(C)COCc2c(C3CC3)nnn2[C@@H]1C(=O)O ; CC1(C)COCc2c(C3CC3)nnn2[C@@H]1C(=O)O ; COC(=O)C[C@H](NC(=O)[C@@H]1C[C@@H](O)CN1)c1ccc(-c2scnc2C)cc1]

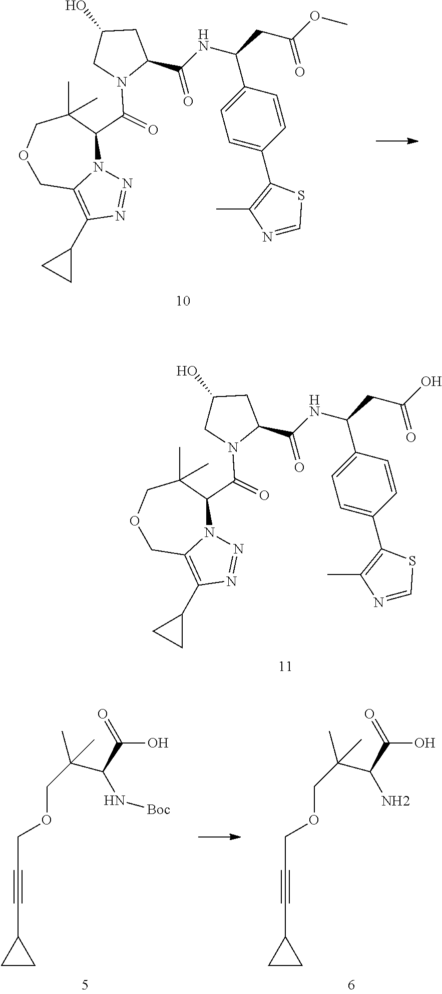 [SMILES: COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; Cc1ncsc1-c1ccc([C@H](CC(=O)O)NC(=O)[C@@H]2C[C@@H](O)CN2C(=O)[C@H]2n3nnc(C4CC4)c3COCC2(C)C)cc1 ; CC(C)(C)OC(=O)N[C@H](C(=O)O)C(C)(C)COCC#CC1CC1 ; CC(C)(COCC#CC1CC1)[C@H](N)C(=O)O]

10% H₂SO₄ (2 mL) was added to a solution of compound 5 (0.1 mmol) in MeCN (0.5 mL, for solubility). and the resulting reaction solution was stirred at rt for overnight. This mixture was purified by reverse phase preparative HPLC to give 6. UPLC-MS calculated [M+H]⁺: 226.1, found: 226.3. Retention time: 1.3 min.

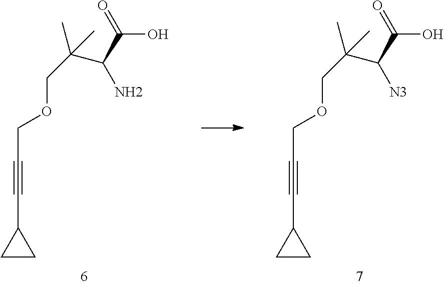 [SMILES: CC(C)(COCC#CC1CC1)[C@H](N)C(=O)O ; CC(C)(COCC#CC1CC1)[C@H](N=[N+]=[N-])C(=O)O]

1H-imidazole-1-sulfonyl azide (0.13 mmol) was added to a mixture of 6 (0.1 mmol), DIEA (0.4 mmol) and cupric sulfate (0.02 mmol) in methanol (1 mL) at 25° C. The reaction mixture was stirred at 25° C. for 0.5-1 h. Purification by reverse phase preparative HPLC gave 7. UPLC-MS calculated [M+H]⁺: 252.2, found: 252.3. Retention time: 3.8 min.

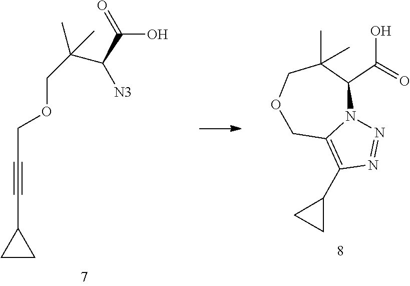 [SMILES: CC(C)(COCC#CC1CC1)[C@H](N=[N+]=[N-])C(=O)O ; CC1(C)COCc2c(C3CC3)nnn2[C@@H]1C(=O)O]

A solution of 7 (30 mg) in 1,2-dichlorobenzene (10 mL) was stirred at 140° C. by microwave for 16 h. Removal of the solvent under vacuum yielded the crude product 10 which was used for the next step without further purification. UPLC-MS calculated [M+H]⁺: 252.1, found: 252.1. Retention time: 1.7 min.

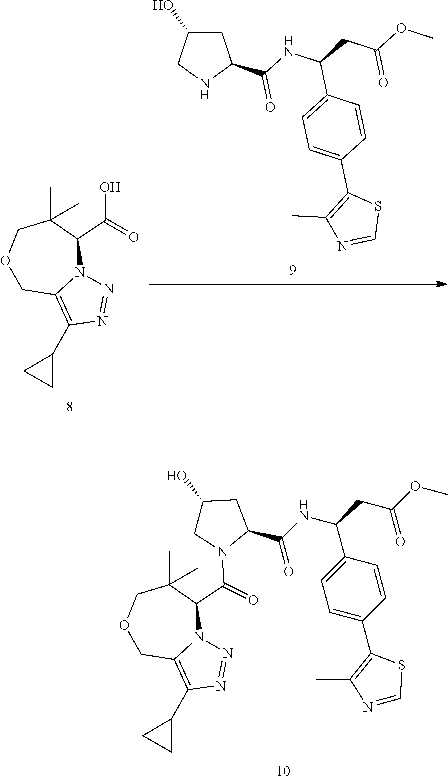 [SMILES: CC1(C)COCc2c(C3CC3)nnn2[C@@H]1C(=O)O ; COC(=O)C[C@H](NC(=O)[C@@H]1C[C@@H](O)CN1)c1ccc(-c2scnc2C)cc1 ; COC(=O)C[C@H](NC(=O)[C@@H]1C[C@@H](O)CN1C(=O)[C@H]1n2nnc(C3CC3)c2COCC1(C)C)c1ccc(-c2scnc2C)cc1]

HATU (0.08 mmol, 1.1 equiv.) was added to a solution of 8 (18 mg, 0.07 mmol, 1 equiv.), 9 (0.07 mmol, 1 equiv.) and DIEA (0.21 mmol, 3 equiv.) in DMF (1 mL) and the resulting mixture was stirred at rt for 1 h. This mixture was purified by reverse phase preparative HPLC to give ZH-7. UPLC-MS calculated [M+H]⁺: 623.3, found: 623.5. Retention time: 3.2 min.

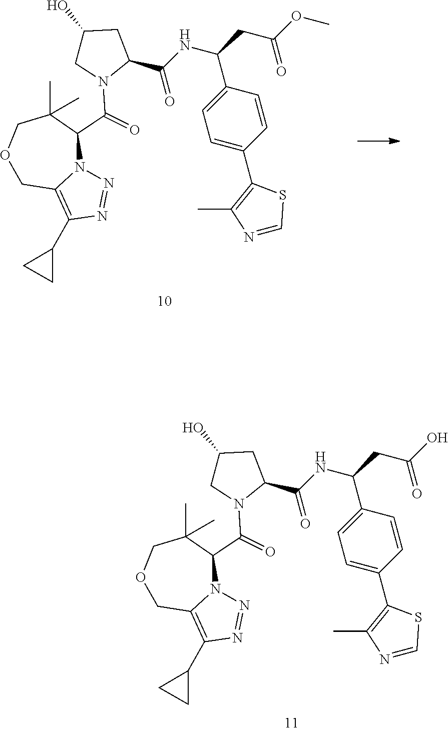 [SMILES: COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; Cc1ncsc1-c1ccc([C@H](CC(=O)O)NC(=O)[C@@H]2C[C@@H](O)CN2C(=O)[C@H]2n3nnc(C4CC4)c3COCC2(C)C)cc1]

LiOH (0.2 mmol in 1 mL H2O) was added to a solution of 10 (0.1 mmoL) in Methanol (1 ml) and the resulting mixture was stirred for 2 h. Acidified with 1M HCl and then purification was performed by HPLC to give 11. UPLC-MS calculated [M+H]⁺: 609.2, found: 609.4. Retention time: 2.6 min.

### Synthesis of Intermediate 18

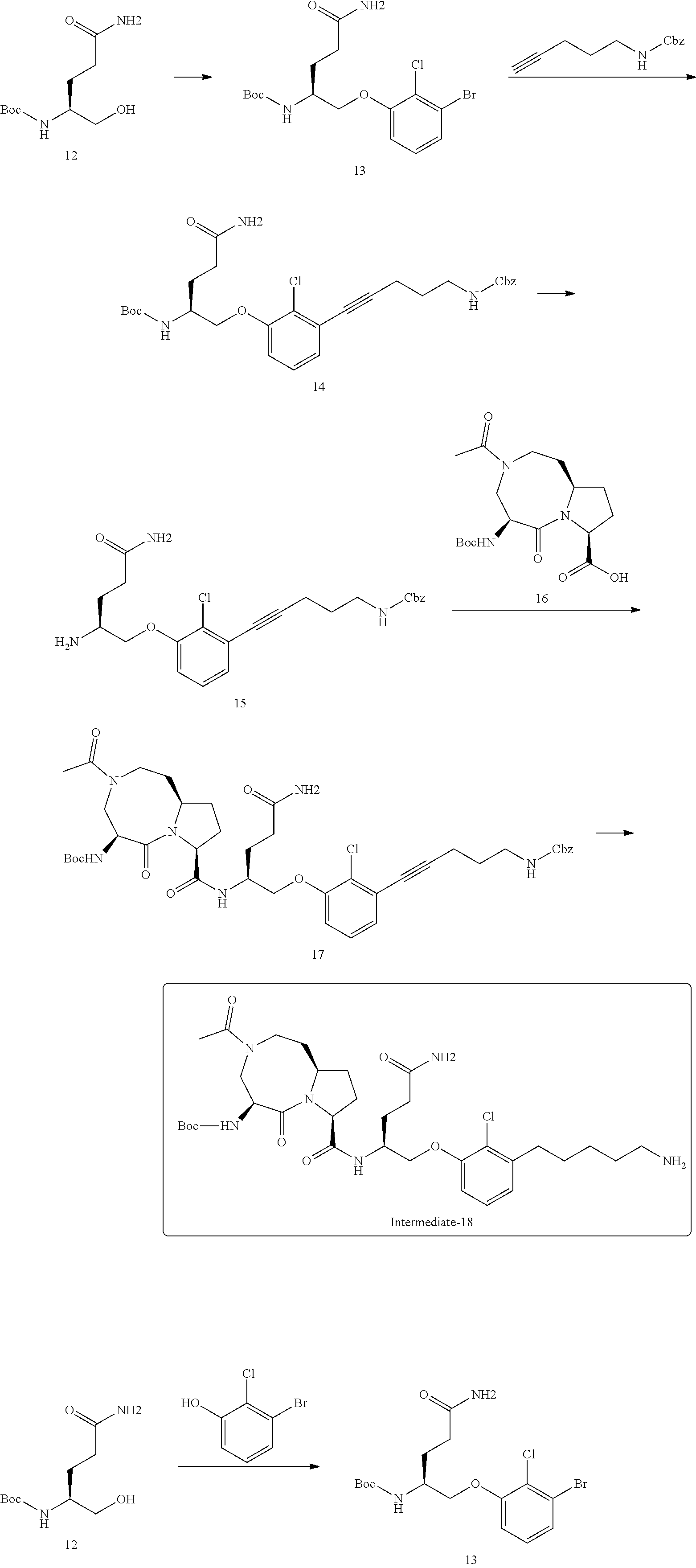

DIAD (1.5 mmol) was added dropwise at 0° C. to the mixture of 12 (1 mmol), 3-bromo-2-chlorophenol (1 mmol) and PPh₃P (1.5 mmol) in THF (10 mL), and the mixture was stirred for 3 h at rt. The reaction mixture was concentrated and purified by reverse phase preparative HPLC. UPLC-MS calculated [M+H]⁺: 421.1, found: 421.5.

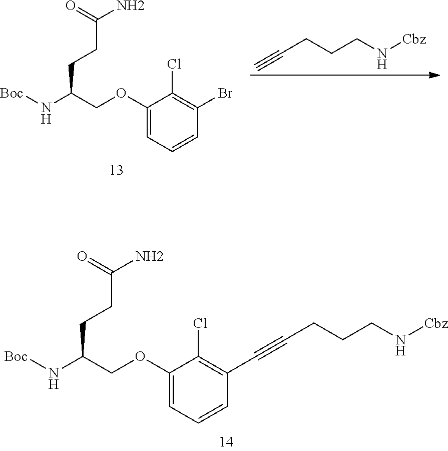

Trimethylamine (3 mL) was added to a mixture of compound 13 (1.0 mmol, 1 equiv.), benzyl pent-4-yn-1-ylcarbamate (1.5 mmol, 1.5 equiv.), CuI (154 mg, 0.2 mmol, 0.2 equiv) and Pd(PPh₃)₂Cl₂ (282 mg, 0.1 mmol, 0.1 equiv) in DMF (5 mL). The resulting mixture was purged and refilled with argon three times and stirred at 70-80° C. for 3 h under Argon. The reaction mixture was then cooled to room temperature and evaporated to remove most of the solvent. The residue was purified by HPLC to yield the 14. UPLC-MS calculated [M+H]⁺: 558.2, found: 558.6. Retention time: 5.1 min.

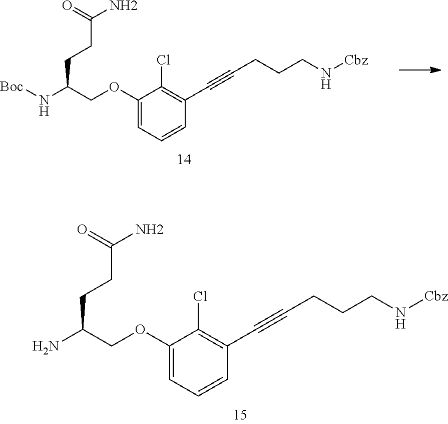 [SMILES: CC(C)(C)OC(=O)N[C@@H](CCC(N)=O)COc1cccc(C#CCCCNC(=O)OCc2ccccc2)c1Cl ; NC(=O)CC[C@H](N)COc1cccc(C#CCCCNC(=O)OCc2ccccc2)c1Cl]

4 M HCl in dioxane was added to a solution of 14 in DCM and the resulting mixture was stirred for 3 h. Removal of the solvent under vacuum to yield the crude product 15 which was used for the next step without further purification. UPLC-MS calculated [M+H]⁺: 458.2, found: 458.4. Retention time: 2.9 min.

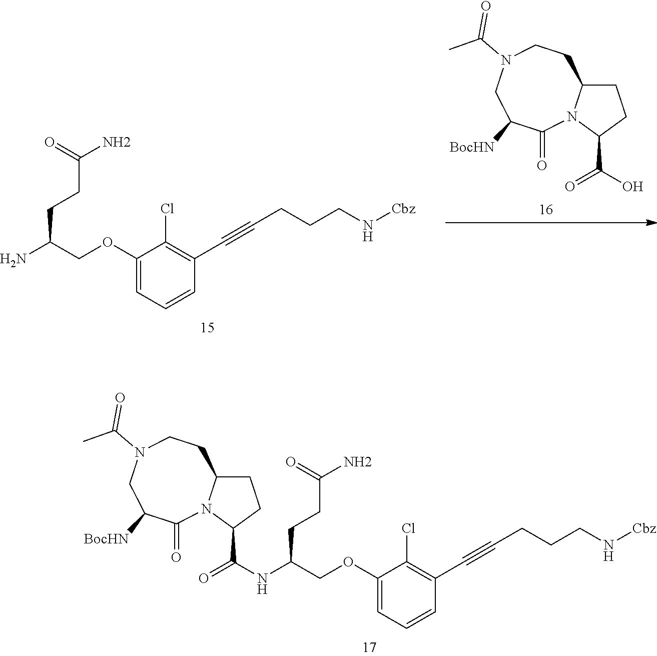 [SMILES: NC(=O)CC[C@H](N)COc1cccc(C#CCCCNC(=O)OCc2ccccc2)c1Cl ; CC(=O)N1CC[C@H]2CC[C@@H](C(=O)O)N2C(=O)[C@@H](NC(=O)OC(C)(C)C)C1 ; CC(=O)N1CC[C@H]2CC[C@@H](C(=O)N[C@@H](CCC(N)=O)COc3cccc(C#CCCCNC(=O)OCc4ccccc4)c3Cl)N2C(=O)[C@@H](NC(=O)OC(C)(C)C)C1]

HATU (0.22 mmol, 1.1 equiv.) was added to a solution of 15 (0.2 mmol, 1 equiv.), 16 (0.2 mmol, 1 equiv.) and DIEA (0.6 mmol, 3 equiv.) in DMF (3 mL) and the resulting mixture was stirred at rt for 1 h. This mixture was purified by reverse phase preparative HPLC to give 17. UPLC-MS calculated [M+H]⁺: 809.4, found: 809.6. Retention time: 4.7 min.

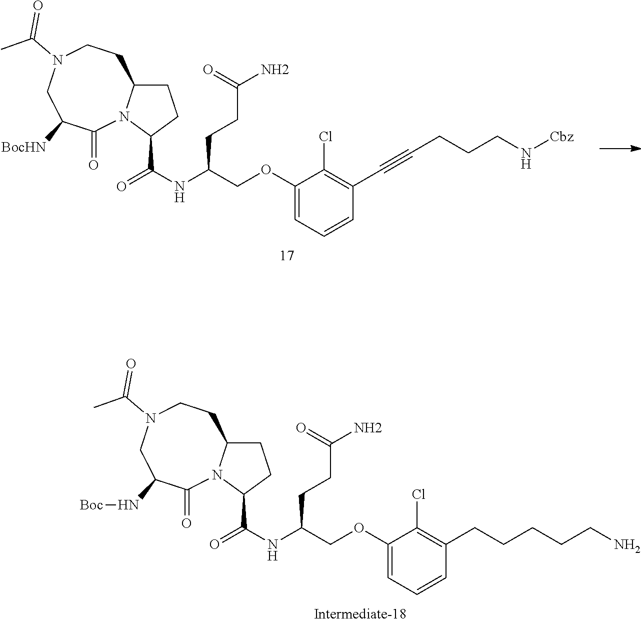 [SMILES: CC(=O)N1CC[C@H]2CC[C@@H](C(=O)N[C@@H](CCC(N)=O)COc3cccc(C#CCCCNC(=O)OCc4ccccc4)c3Cl)N2C(=O)[C@@H](NC(=O)OC(C)(C)C)C1 ; CC(=O)N1CC[C@H]2CC[C@@H](C(=O)N[C@@H](CCC(N)=O)COc3cccc(CCCCCN)c3Cl)N2C(=O)[C@@H](NC(=O)OC(C)(C)C)C1]

To a solution of compound 17 (0.1 mmol) in MeOH (4 mL) was added platinum(iv) oxide (15 mg). The mixture was stirred under 1 atm of H2 at room temperature for 3 hours before filtering through celite and being concentrated. The resulting amine was used for the next step without further purification. UPLC-MS calculated [M+H]⁺: 679.4, found: 679.7. Retention time: 2.4 min.

Final steps of synthesis of BJ

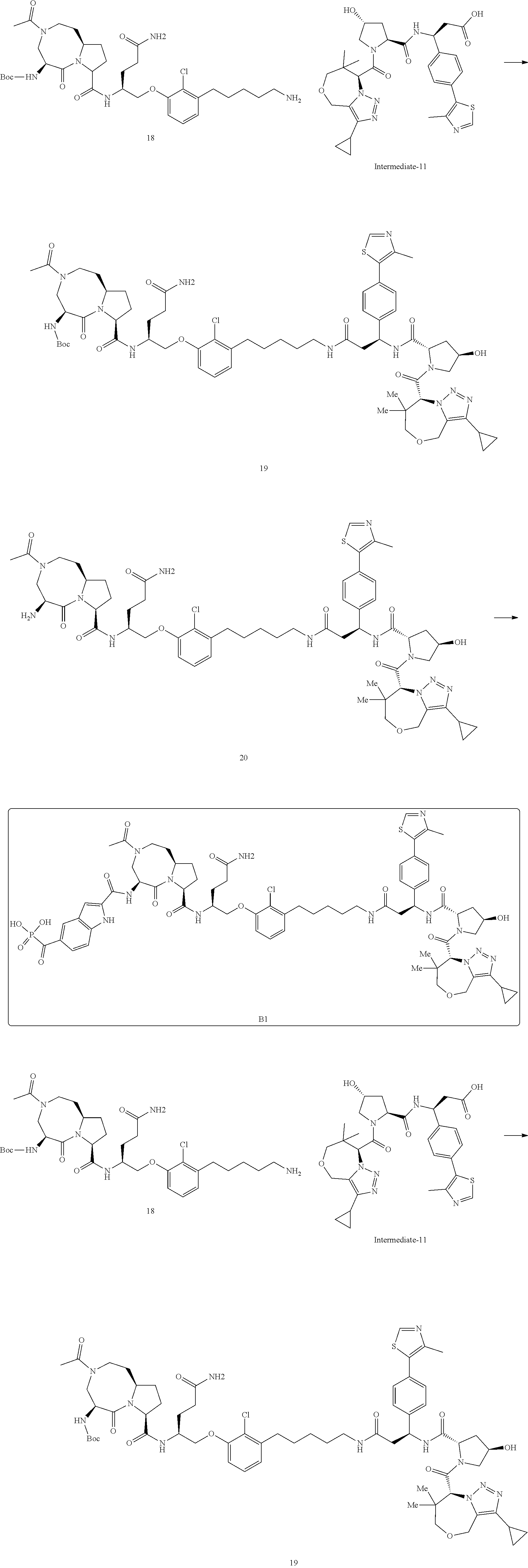

HATU (0.022 mmol, 1.1 equiv.) was added to a solution of Intermediate-11 (0.02 mmol, 1 equiv.), 18 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield 19. UPLC-MS calculated [M+2H]²⁺: 635.3, found: 635.4. Retention time: 4.2 min.

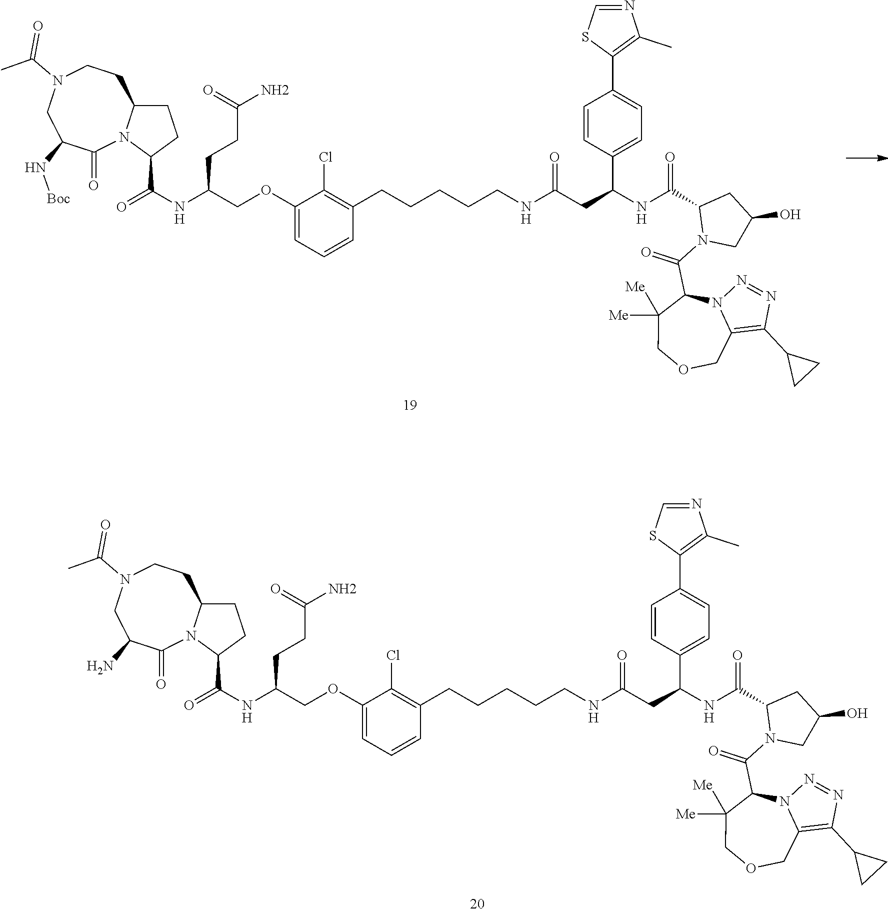 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)OC(C)(C)C)C1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](N)C1]

Compound 19 (20 mg) was treated with 1 mL of 1M HCl (in Methanol) and 1 mL of 4M HCl (in dioxane) for 2 h. The reaction mixture was evaporated to give the 20 without further purification. UPLC-MS calculated [M+2H]²⁺: 635.3, found: 635.4. Retention time: 3.0 min.

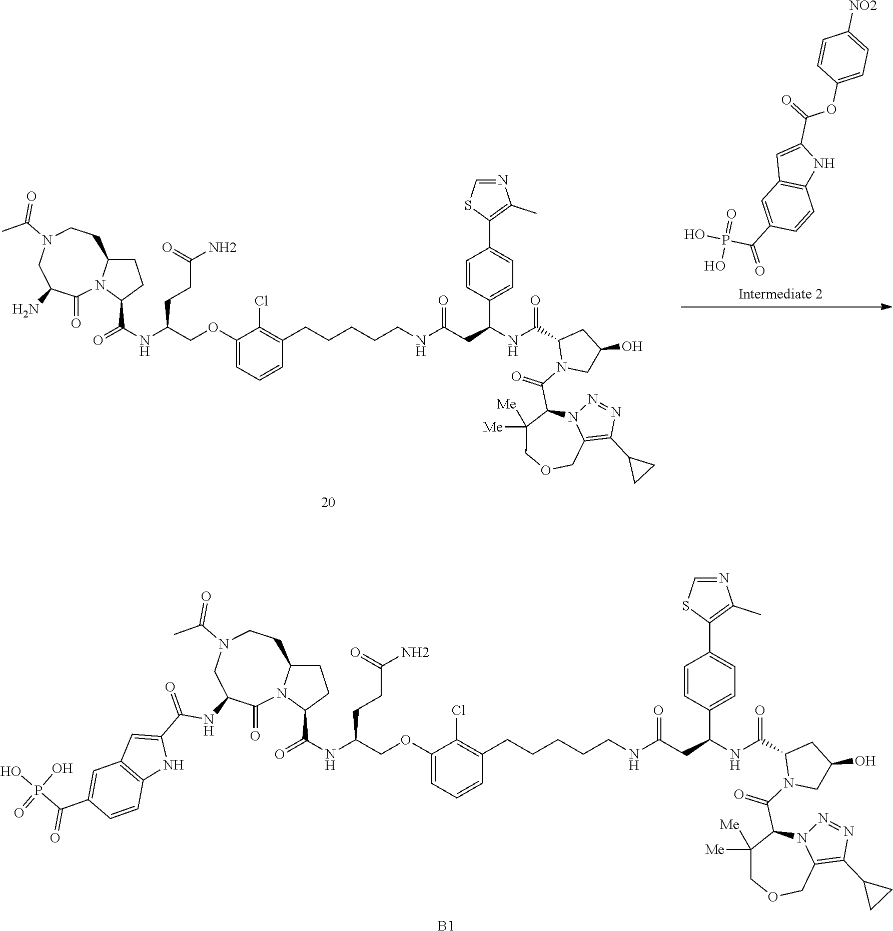 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](N)C1 ; O=C(Oc1ccc([N+](=O)[O-])cc1)c1cc2cc(C(=O)P(=O)(O)O)ccc2[nH]1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1 ; "B1"]

DIEA (0.042 mmol, 3 equiv.) was added to the mixture of Intermediate 2 (0.017 mmol, 1.2 equiv.), compound 20 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B1. UPLC-MS calculated [M+2H]²⁺: 710.8, found: 710.9. Retention time: 3.4 min.

### Compound B2. (2-(((3S,6S,10aS)-3-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-5-oxodecahydropyrrolo[1,2-a]azocin-6-yl)carbamoyl)-1H-indole-5-carbonyl)phosphonic acid

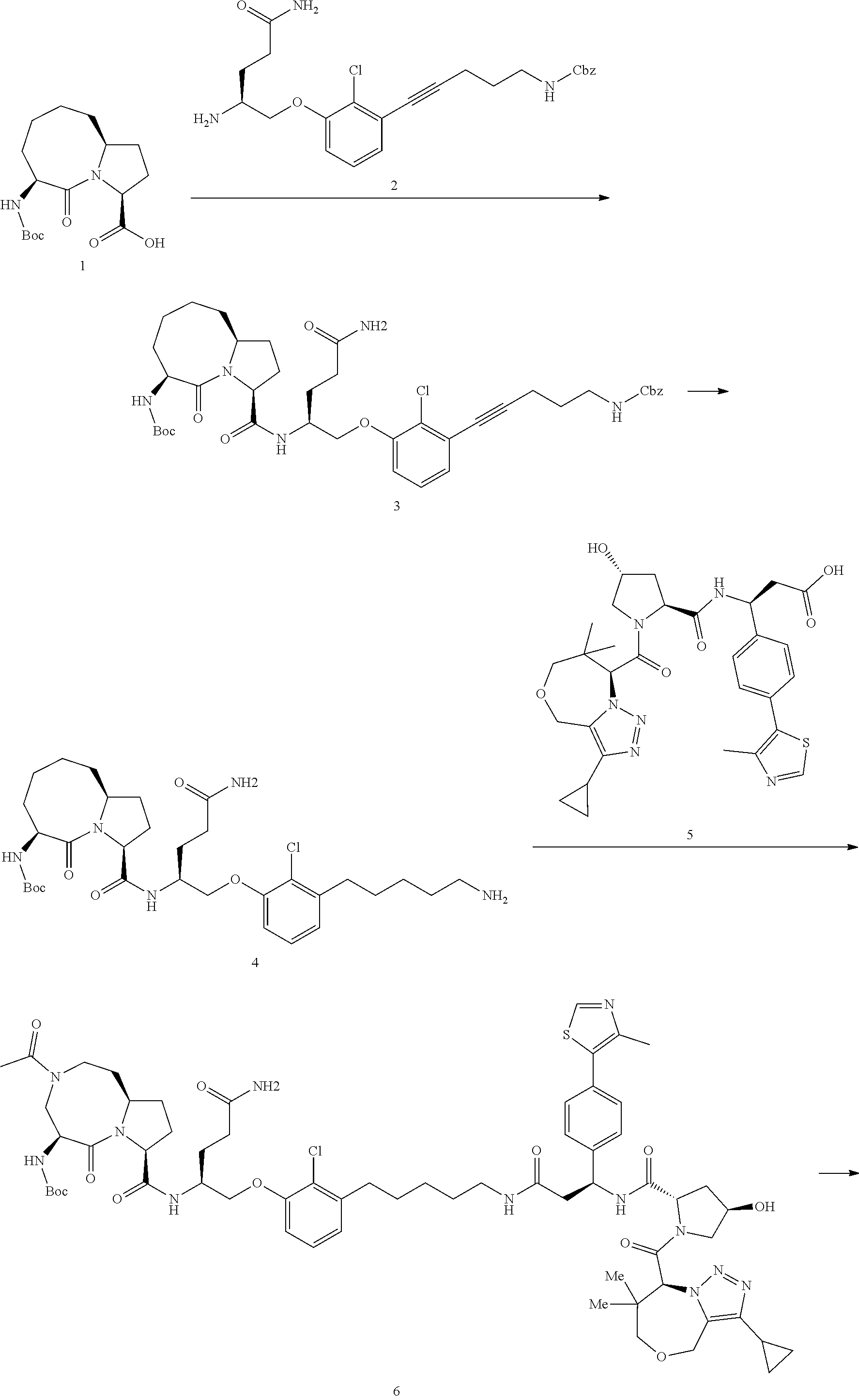

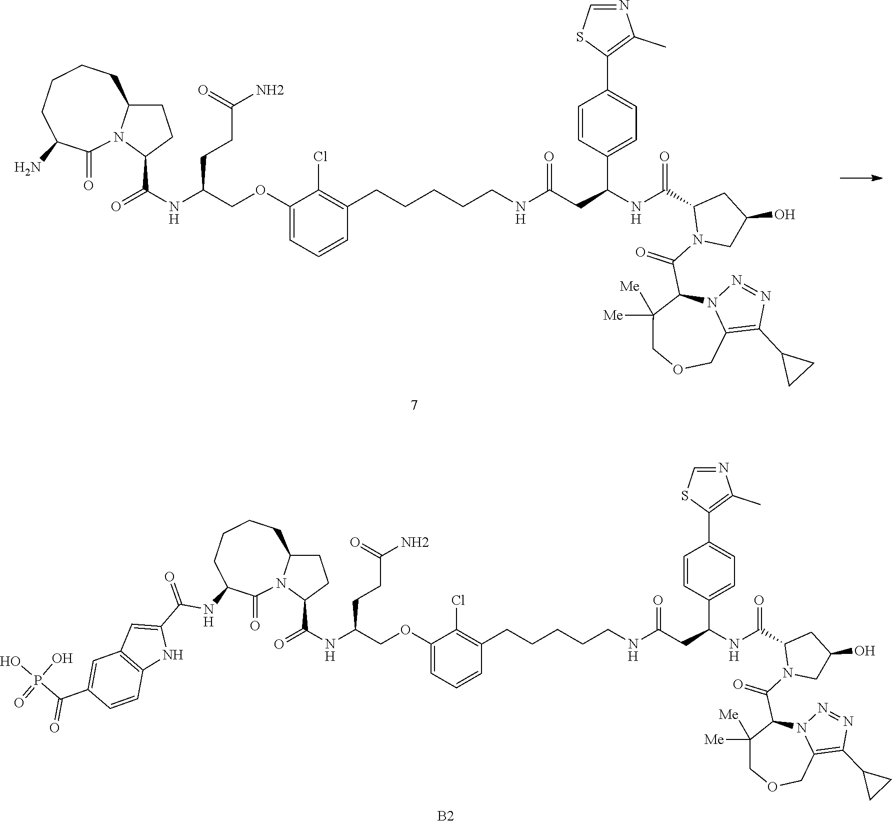 [SMILES: Cc1ncsc1-c1ccc([C@H](CC(=O)NCCCCCc2cccc(OC[C@H](CCC(N)=O)NC(=O)[C@@H]3CC[C@@H]4CCCC[C@H](N)C(=O)N43)c2Cl)NC(=O)[C@@H]2C[C@@H](O)CN2C(=O)[C@H]2n3nnc(C4CC4)c3COCC2(C)C)cc1 ; Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)NC(=O)[C@@H]2C[C@@H](O)CN2C(=O)[C@H]2n3nnc(C4CC4)c3COCC2(C)C)cc1 ; "B2"]

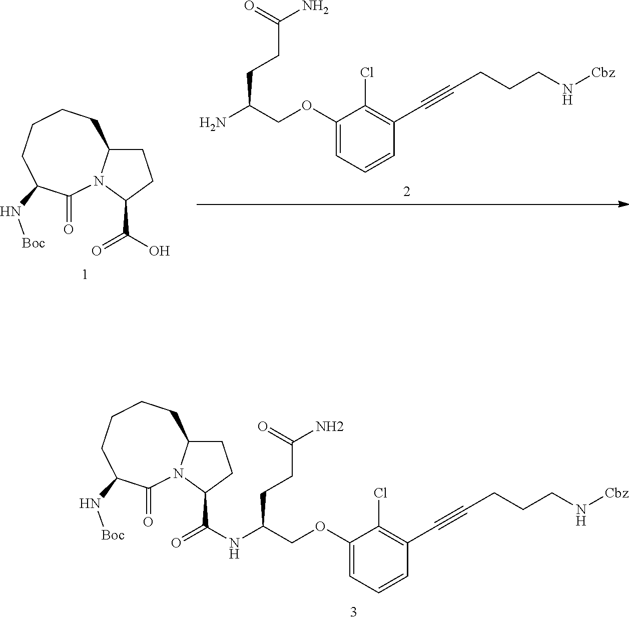 [SMILES: CC(C)(C)OC(=O)N[C@H]1CCCC[C@H]2CC[C@@H](C(=O)O)N2C1=O ; NC(=O)CC[C@H](N)COc1cccc(C#CCCCNC(=O)OCc2ccccc2)c1Cl ; CC(C)(C)OC(=O)N[C@H]1CCCC[C@H]2CC[C@@H](C(=O)N[C@@H](CCC(N)=O)COc3cccc(C#CCCCNC(=O)OCc4ccccc4)c3Cl)N2C1=O]

HATU (0.22 mmol, 1.1 equiv.) was added to a solution of 1 (0.2 mmol, 1 equiv.), 2 (0.2 mmol, 1 equiv.) and DIEA (0.6 mmol, 3 equiv.) in DMF (3 mL) and the resulting mixture was stirred at rt for 1 h. This mixture was purified by reverse phase preparative HPLC to give 3. UPLC-MS calculated [M+H]⁺: 766.4, found: 766.6. Retention time: 5.4 min.

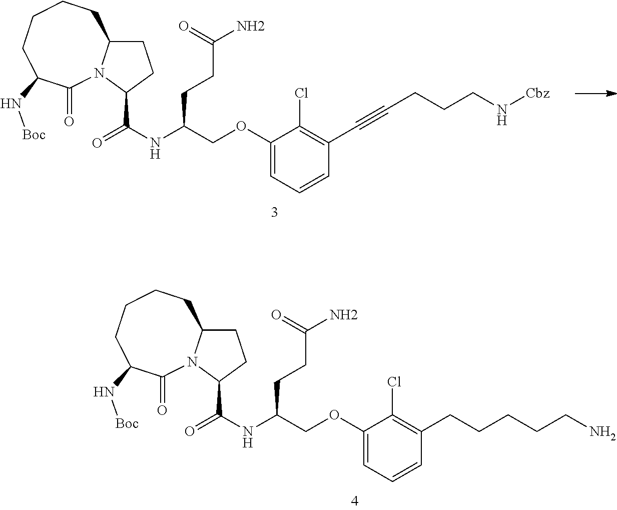 [SMILES: CC(C)(C)OC(=O)N[C@H]1CCCC[C@H]2CC[C@@H](C(=O)N[C@@H](CCC(N)=O)COc3cccc(C#CCCCNC(=O)OCc4ccccc4)c3Cl)N2C1=O ; CC(C)(C)OC(=O)N[C@H]1CCCC[C@H]2CC[C@@H](C(=O)N[C@@H](CCC(N)=O)COc3cccc(CCCCCN)c3Cl)N2C1=O]

To a solution of compound 3 (0.1 mmol) in MeOH (4 mL) was added platinum(iv) oxide (15 mg). The mixture was stirred under 1 atm of H₂ at room temperature for 3 hours before filtering through celite and being concentrated. The resulting amine was used for the next step without further purification. UPLC-MS calculated [M+H]⁺: 636.3, found: 636.7 Retention time: 3.5 min.

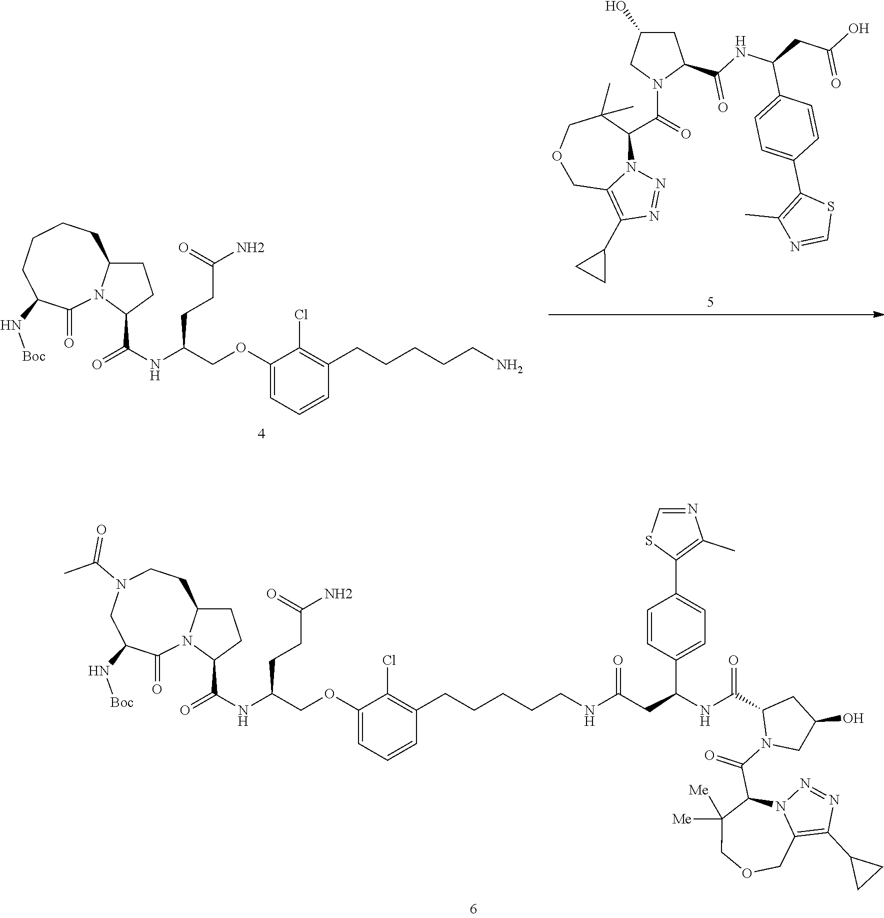 [SMILES: CC(C)(C)OC(=O)N[C@H]1CCCC[C@H]2CC[C@@H](C(=O)N[C@@H](CCC(N)=O)COc3cccc(CCCCCN)c3Cl)N2C1=O ; Cc1ncsc1-c1ccc([C@H](CC(=O)O)NC(=O)[C@@H]2C[C@@H](O)CN2C(=O)[C@H]2n3nnc(C4CC4)c3COCC2(C)C)cc1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)OC(C)(C)C)C1]

HATU (0.033 mmol, 1.1 equiv.) was added to a solution of 4 (0.03 mmol, 1 equiv.), 5 (0.03 mmol, 1 equiv.) and DIEA (0.09 mmol, 3 equiv.) in DMF (1 mL) and the resulting mixture was stirred at rt for 1 h. This mixture was purified by reverse phase preparative HPLC to yield 6. UPLC-MS calculated [M+2H]²⁺: 613.8, found: 613.9. Retention time: 4.8 min.

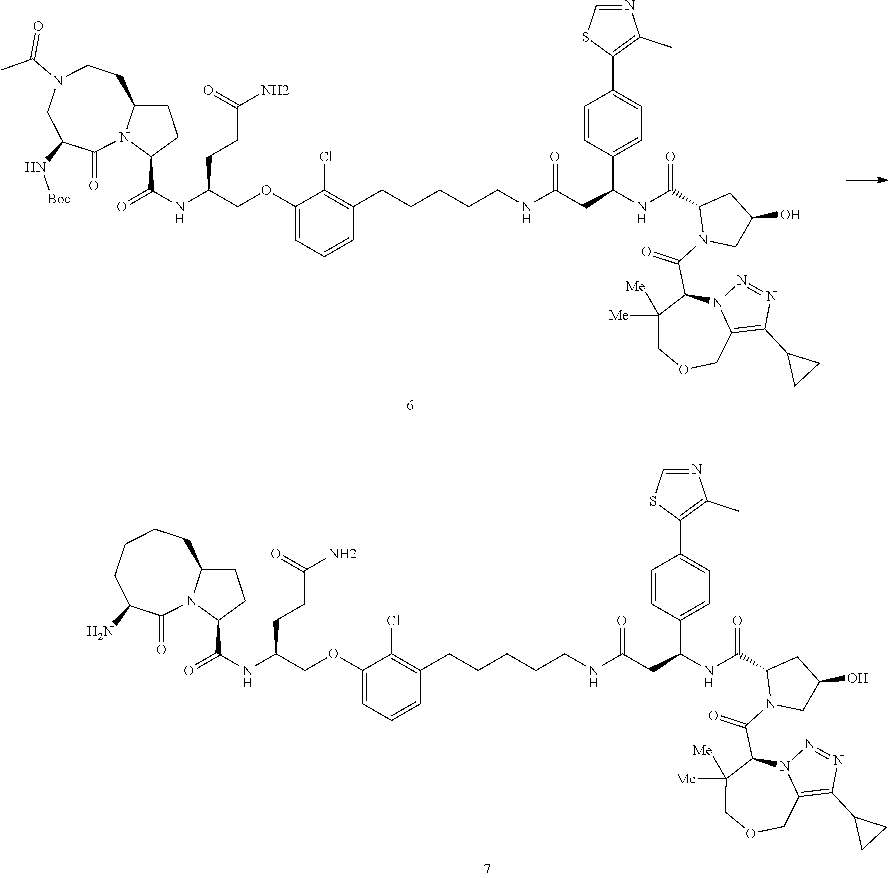 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)OC(C)(C)C)C1 ; Cc1ncsc1-c1ccc([C@H](CC(=O)NCCCCCc2cccc(OC[C@H](CCC(N)=O)NC(=O)[C@@H]3CC[C@@H]4CCCC[C@H](N)C(=O)N43)c2Cl)NC(=O)[C@@H]2C[C@@H](O)CN2C(=O)[C@H]2n3nnc(C4CC4)c3COCC2(C)C)cc1]

Compound 6 (20 mg) was treated with 1 mL of 1M HCl (in Methanol) and 1 mL of 4M HCl (in dioxane) for 2 h. The reaction mixture was evaporated to give the 7 without further purification. UPLC-MS calculated [M+H]⁺: 1126.5, found: 1126.1. Retention time: 3.6 min.

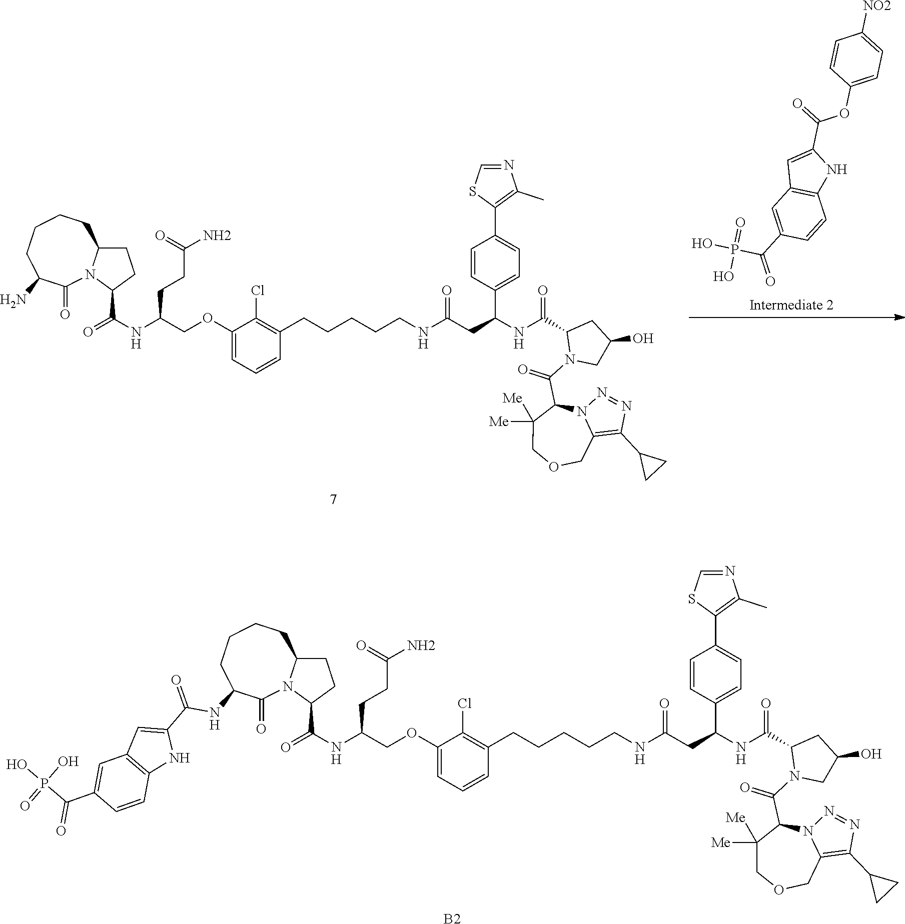 [SMILES: Cc1ncsc1-c1ccc([C@H](CC(=O)NCCCCCc2cccc(OC[C@H](CCC(N)=O)NC(=O)[C@@H]3CC[C@@H]4CCCC[C@H](N)C(=O)N43)c2Cl)NC(=O)[C@@H]2C[C@@H](O)CN2C(=O)[C@H]2n3nnc(C4CC4)c3COCC2(C)C)cc1 ; O=C(Oc1ccc([N+](=O)[O-])cc1)c1cc2cc(C(=O)P(=O)(O)O)ccc2[nH]1 ; Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(=O)P(=O)(O)O)ccc6[nH]5)C(=O)N43)c2Cl)NC(=O)[C@@H]2C[C@@H](O)CN2C(=O)[C@H]2n3nnc(C4CC4)c3COCC2(C)C)cc1 ; "B2"]

DIEA (0.06 mmol, 3 equiv.) was added to the mixture of Intermediate 2 (0.024 mmol, 1.2 equiv.), compound 7 (0.02 mmol, 1 equiv.) and HOBt (0.04 mmol, 2 equiv.) in DMF (1 mL). The resulted mixture was stirred at room temperature for 1 hour. Purification of this reaction mixture by HPLC gave the compound B2. UPLC-MS calculated [M+2H]²⁺: 689.3, found: 689.3.

### Compound B3. (2-(((5S,8S,10aR)-3-acetyl-8-(((S)-5-amino-1-(2-chloro-3-(3-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-7,8-dihydro-4H,6H-[1,2,3]triazolo[5,1-c][1,4]oxazepin-3-yl)propyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

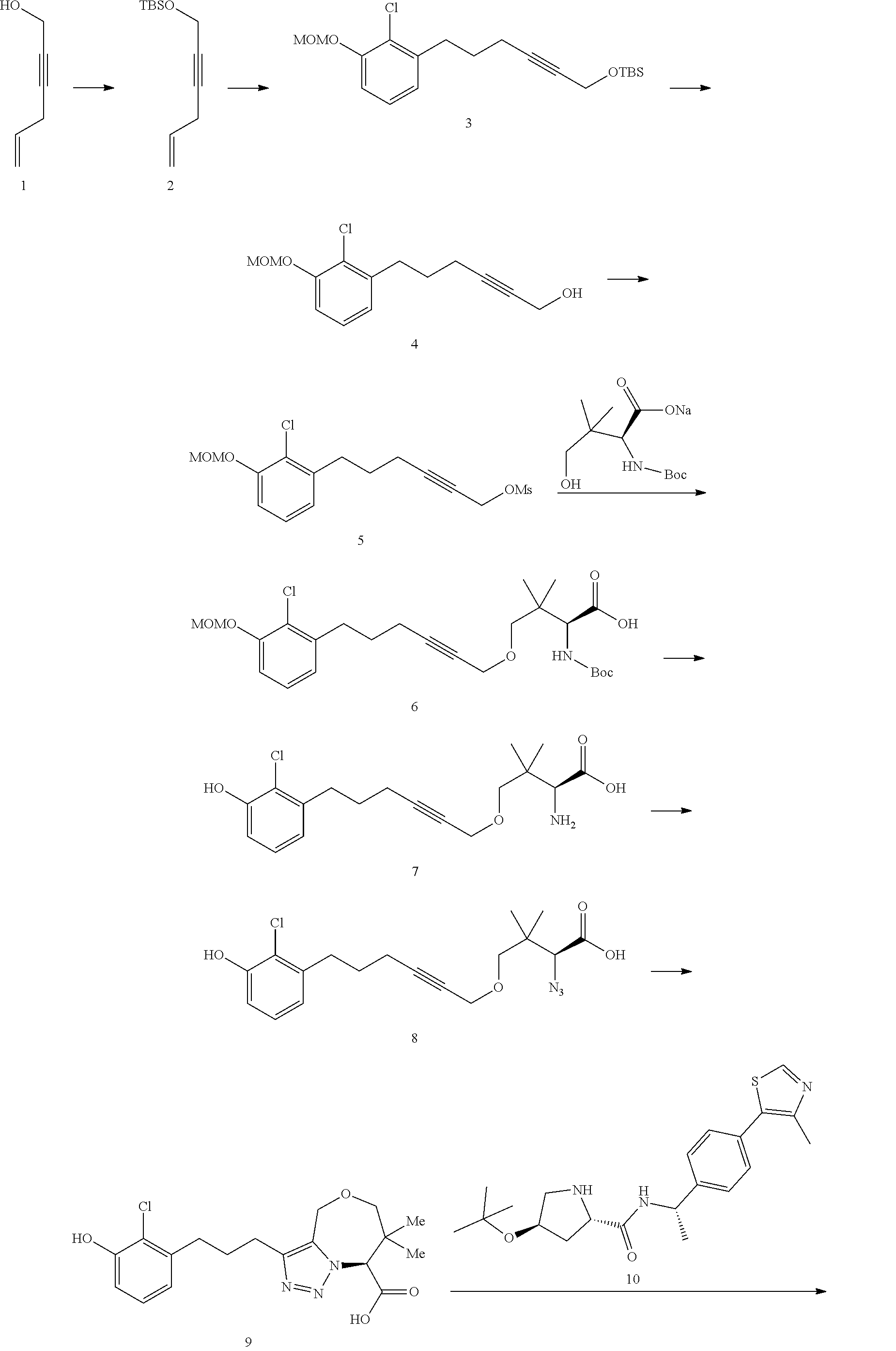

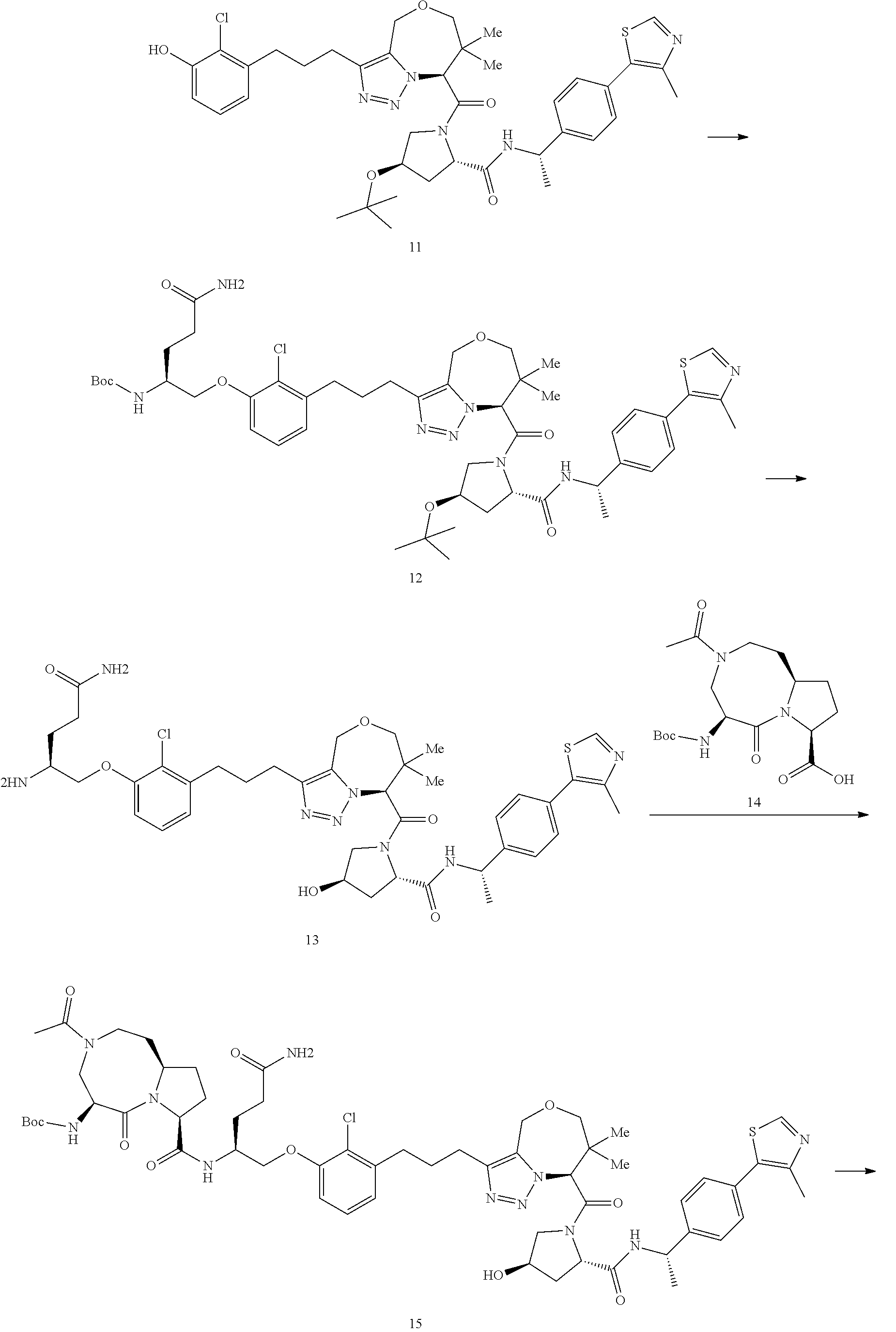

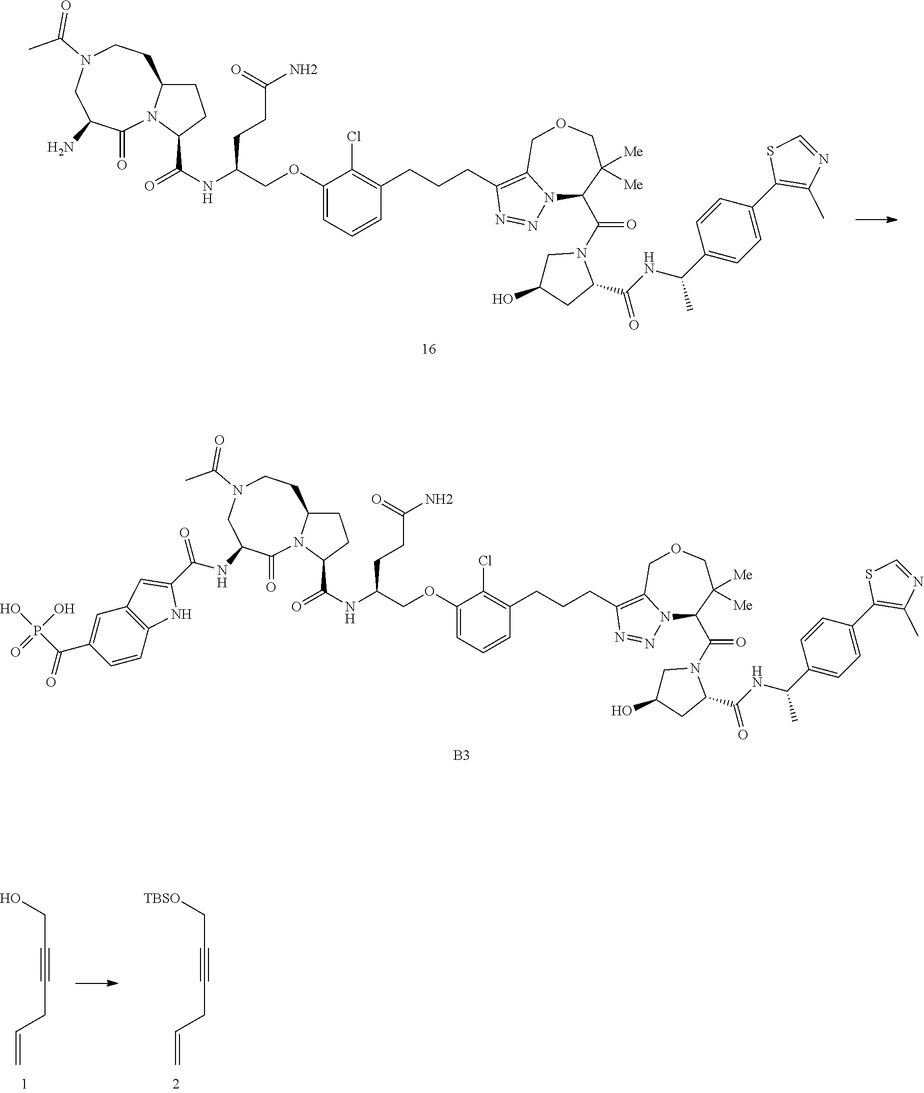

To a solution of 1 (4.8 g, 50 mmol, 1 eq) and imidazole (75 mmol, 1.5 eq) in CH₂Cl₂ (10 ml) at room temperature was added TBSCl (55 mmol, 1.1 eq). The reaction mixture was stirred for 1 h, then quenched with water and extracted with EtOAc. The organic layer was washed with brine, dried over MgSO₄, and concentrated under reduced pressure. The residue was purified by column chromatography on silica gel eluting with EtOAc-hexane to give 2.

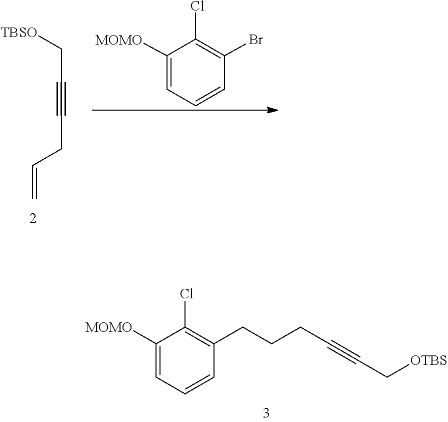

9-BBN in THF (0.5m, 22 mL, 11 mmol, 1.2 eq) was added to neat 2 (9.17 mmol, 1 eq.) at 0° C. The resulted mixture was stirred at rt overnight. NaOH aq (4M, 9.2 mL, 36.7 mmol, 4 eq) was added and stir for 30 min at rt. 3-bromo-2-chlorophenol (2.3 g, 9.17 mmol, 1 eq) and Pd(dppf)Cl₂ (748 mg, 0.92 mmol, 0.1 eq) were added and stir at 50-60° C. for 1 hour, the reaction mixture was quenched with saturated aqueous NaHCO₃, extracted with EA, washed with brine, dried over MgSO₄, and concentrated. Column chromatography (silica gel, hexanes/EA) afforded 3.

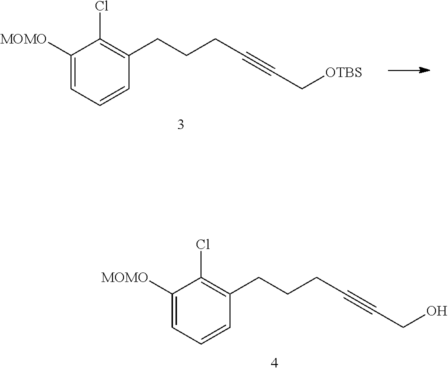

Tetrabutylammonium fluoride solution (4.8 mL, 1M in THF, 1.5 eq) was added to the solution of 3 (1.2 g, 3.2 mmol) in THF (5 mL). The reaction mixture was stirred for 2 h and H2O (5 mL) was added. The resulting mixture was extracted with EtOAc and the combined organic phases were washed with brine. The organic layer was dried over Na₂SO₄, filtered and concentrated in vacuo. The residue was purified by column chromatography (silica gel, hexanes/EA) to afforded 4.

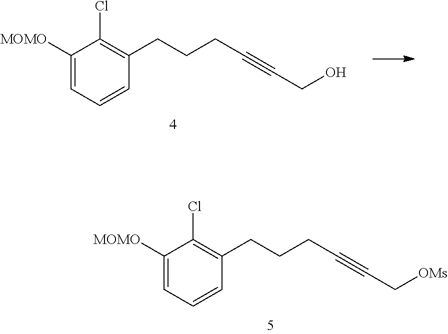

TEA (0.19 mL, 1.1 eq) and methanesulfonyl chloride (156 mg, 1 eq) were added to the solution of alcohol 4 (366 mg, 1.36 mmol, 1 eq) in diethyl either (10 mL) at 0° C. The resulted mixture was stirred at 0° C. for 1 h until the starting material was consumed. 5 ml of water was added and extract with diethyl either twice. The organic layer was dried over Na₂SO₄, filtered and concentrated in vacuo to afford crude 5 which can be purified by column chromatography.

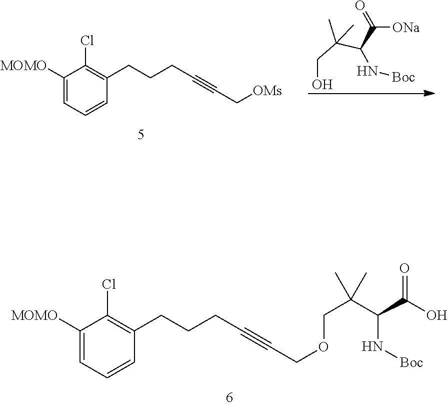

Sodium (S)-3-((tert-butoxycarbonyl)amino)-3-carboxy-2,2-dimethylpropan-1-olate (182 mg, 0.68 mmol, 1.2 equiv.) was dissolved in DMF (4 ml). The solution was cooled to 0° C. and NaH (41 mg, 60 wt-% suspension in mineral oil, 1.8 equiv) was added in portions. After stirring for 0.5-1 h at 0° C., 5 (195 mg, 1 eq) in DMF (2 mL) was added dropwise. The reaction was stirred at 0° C. for 1 h. The reaction was quenched by addition of 1 mL water, and then acidified with 1M KHSO4 to pH 1-2. The resulting mixture was extracted with EtOAc and the combined organic phases were washed with brine. The organic layer was dried over Na₂SO₄, filtered and concentrated in vacuo to afford crude 5 which can be purified by HPLC to give 6. UPLC-MS calculated [M+Na]⁺: 520.2, found: 520.3. Retention time: 6.3 min.

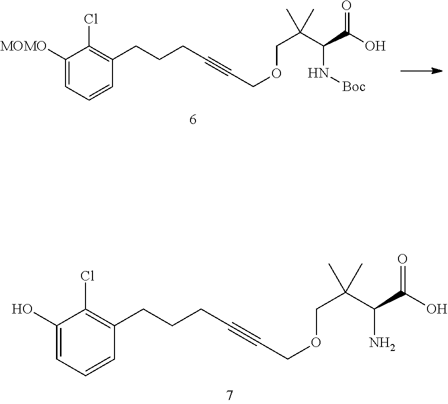

10% H₂SO₄ (4 mL) was added to a solution of compound 6 (0.2 mmol) in MeCN (1 mL, for solubility). and the resulting reaction solution was stirred at rt for overnight. This mixture was purified by reverse phase preparative HPLC to give 7. UPLC-MS calculated [M+H]⁺: 354.1, found: 354.3. Retention time: 3.1 min.

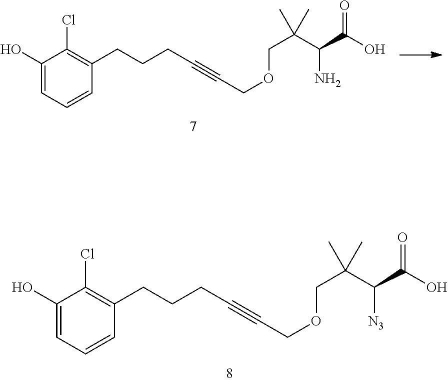 [SMILES: CC(C)(COCC#CCCCc1cccc(O)c1Cl)[C@H](N)C(=O)O ; CC(C)(COCC#CCCCc1cccc(O)c1Cl)[C@H](N=[N+]=[N-])C(=O)O]

1H-imidazole-1-sulfonyl azide (0.24 mmol, 1.3 eq) was added to a mixture of 7 (64 mg, 0.18 mmol, 1 eq), DIEA (0.73 mmol) and cupric sulfate (0.036 mmol, 0.2 eq) in methanol (1 mL) at 25° C. The reaction mixture was stirred at 25° C. for 0.5-1 h. Purification by reverse phase preparative HPLC gave 8. UPLC-MS calculated [M+H]⁺: 380.1, found: 380.4. Retention time: 5.2 min.

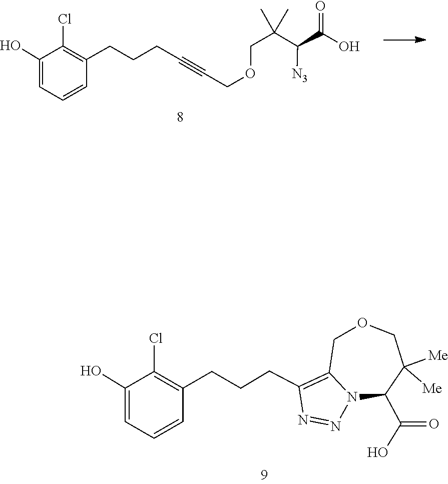 [SMILES: CC(C)(COCC#CCCCc1cccc(O)c1Cl)[C@H](N=[N+]=[N-])C(=O)O ; CC1(C)COCc2c(CCCc3cccc(O)c3Cl)nnn2[C@@H]1C(=O)O]

A solution of 8 (33 mg) in 1,2-dichlorobenzene (10 mL) was stirred at 140° C. by microwave for 18 h. Removal of the solvent under vacuum yielded the crude product 10 which was used for the next step without further purification. UPLC-MS calculated [M+H]⁺: 380.1, found: 380.4. Retention time: 3.3 min.

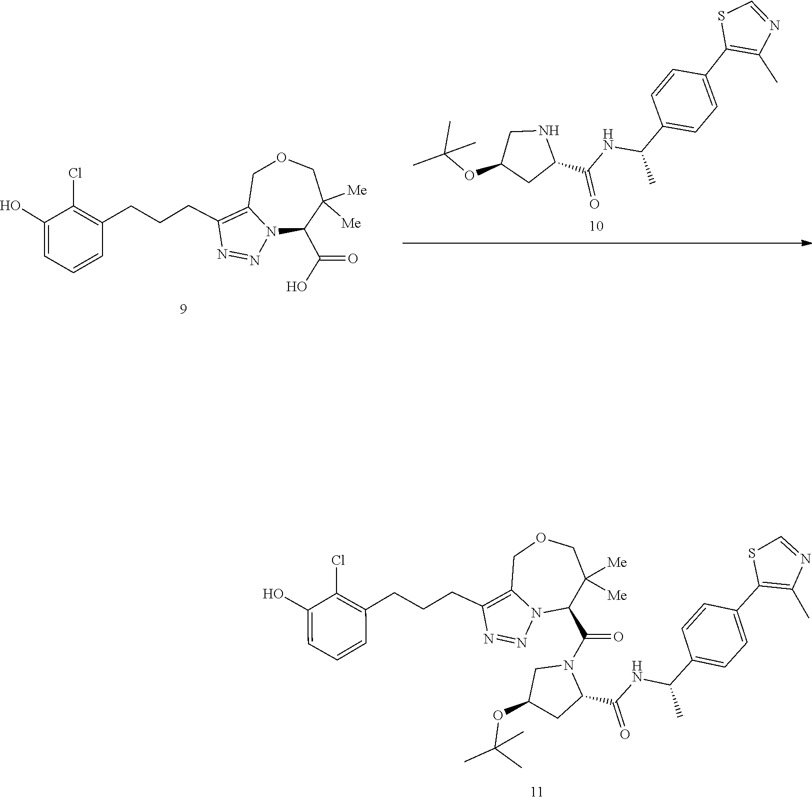 [SMILES: CC1(C)COCc2c(CCCc3cccc(O)c3Cl)nnn2[C@@H]1C(=O)O ; Cc1ncsc1-c1ccc([C@H](C)NC(=O)[C@@H]2C[C@@H](OC(C)(C)C)CN2)cc1 ; Cc1ncsc1-c1ccc([C@H](C)NC(=O)[C@@H]2C[C@@H](OC(C)(C)C)CN2C(=O)[C@H]2n3nnc(CCCc4cccc(O)c4Cl)c3COCC2(C)C)cc1]

HATU (0.087 mmol, 1.1 equiv.) was added to a solution of 9 (33 mg, 0.087 mmol, 1 equiv.), 10 (0.087 mmol, 1 equiv.) and DIEA (0.26 mmol, 3 equiv.) in DMF (2 mL) and the resulting mixture was stirred at rt for 1 h. This mixture was purified by reverse phase preparative HPLC to give 11. UPLC-MS calculated [M+H]⁺: 749.3, found: 749.5. Retention time: 5.4 min.

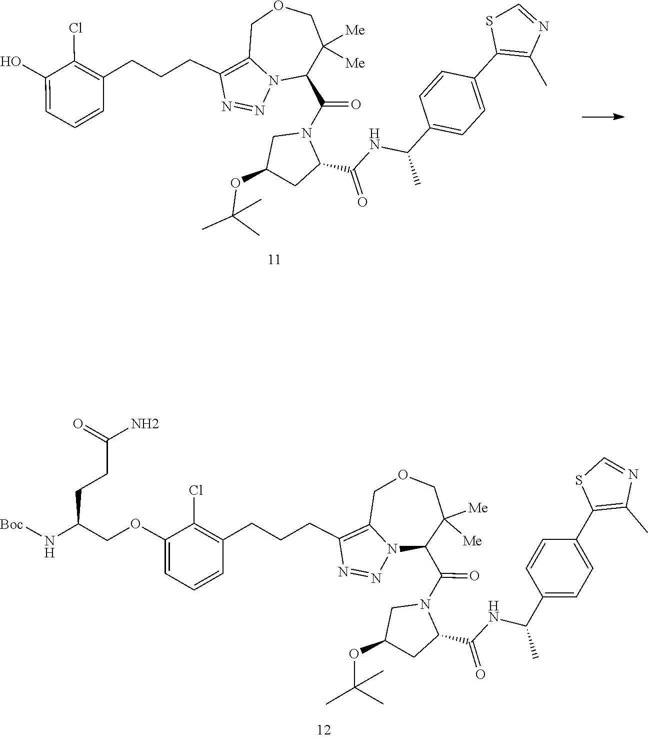 [SMILES: Cc1ncsc1-c1ccc([C@H](C)NC(=O)[C@@H]2C[C@@H](OC(C)(C)C)CN2C(=O)[C@H]2n3nnc(CCCc4cccc(O)c4Cl)c3COCC2(C)C)cc1 ; Cc1ncsc1-c1ccc([C@H](C)NC(=O)[C@@H]2C[C@@H](OC(C)(C)C)CN2C(=O)[C@H]2n3nnc(CCCc4cccc(OC[C@H](CCC(N)=O)NC(=O)OC(C)(C)C)c4Cl)c3COCC2(C)C)cc1]

DIAD (0.17 mmol, 2 eq) was added dropwise at 0° C. to the mixture of 11 (0.086 mmol, 1 eq), tert-butyl (S)-(5-amino-1-hydroxy-5-oxopentan-2-yl)carbamate (0.17 mmol, 2 eq) and PPh₃P (0.17 mmol, 2 eq) in THF (2 mL), and the mixture was stirred for 3 h at rt. The reaction mixture was concentrated and purified by reverse phase preparative HPLC to give 12. UPLC-MS calculated [M+H]⁺: 963.4, found: 963.2. Retention time: 5.6 min.

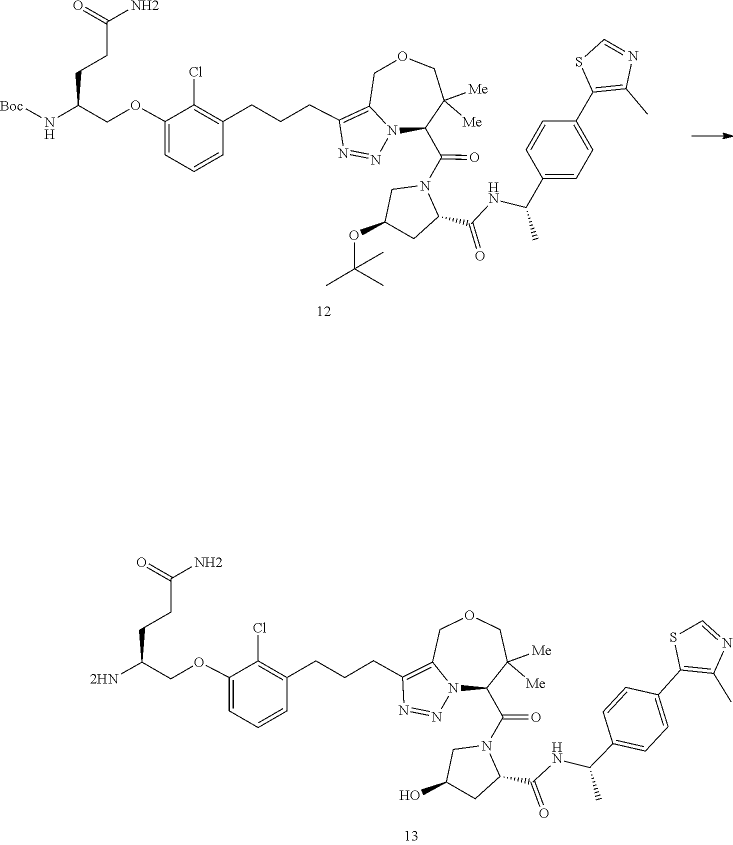

Compound 12 (25 mg) was treated with 1 mL of 1M HCl (in Methanol) and 1 mL of 4M HCl (in dioxane) for 2 h. The reaction mixture was evaporated to give the 13 without further purification. UPLC-MS calculated [M+H]⁺: 807.3, found: 807.6.

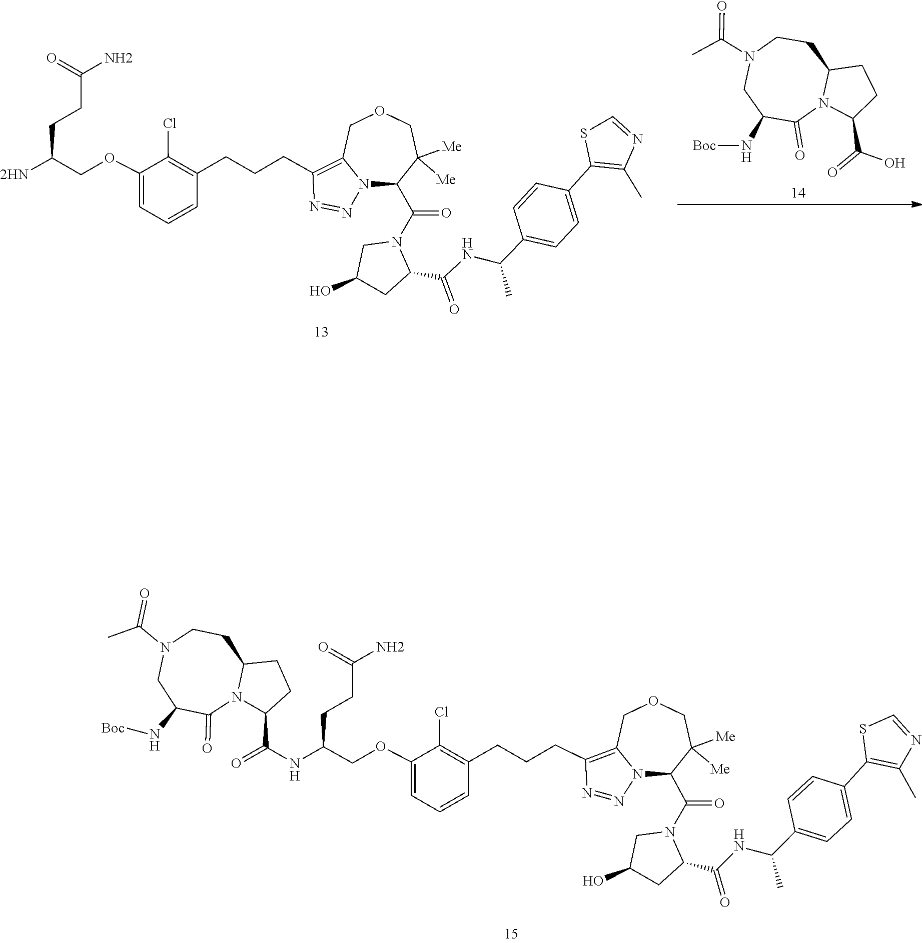

HATU (0.022 mmol, 1.1 equiv.) was added to a solution of 11 (0.02 mmol, 1 equiv.), 14 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield 15. UPLC-MS calculated [M+H]⁺: 1158.5, found: 1158.1. Retention time: 4.4 min.

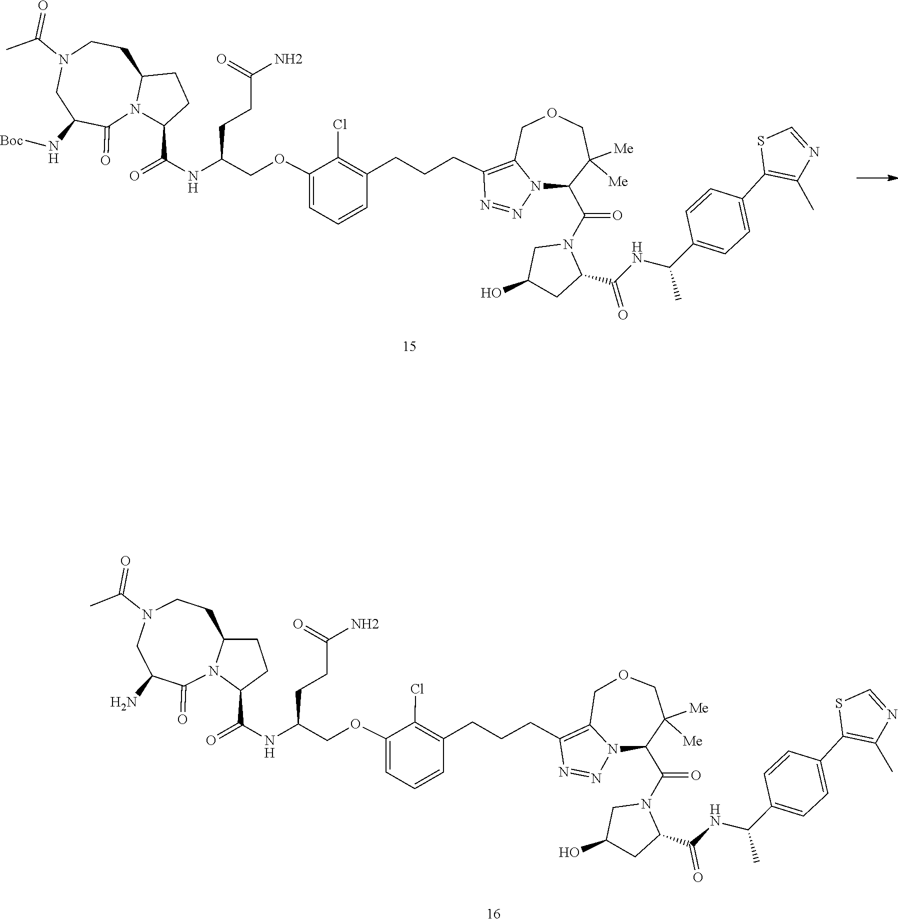

Compound 15 (20 mg) was treated with 1 mL of 1M HCl (in Methanol) and 1 mL of 4M HCl (in dioxane) for 2 h. The reaction mixture was evaporated to give the 16 without further purification. UPLC-MS calculated [M+H]⁺: 1058.5, found: 1058.8.

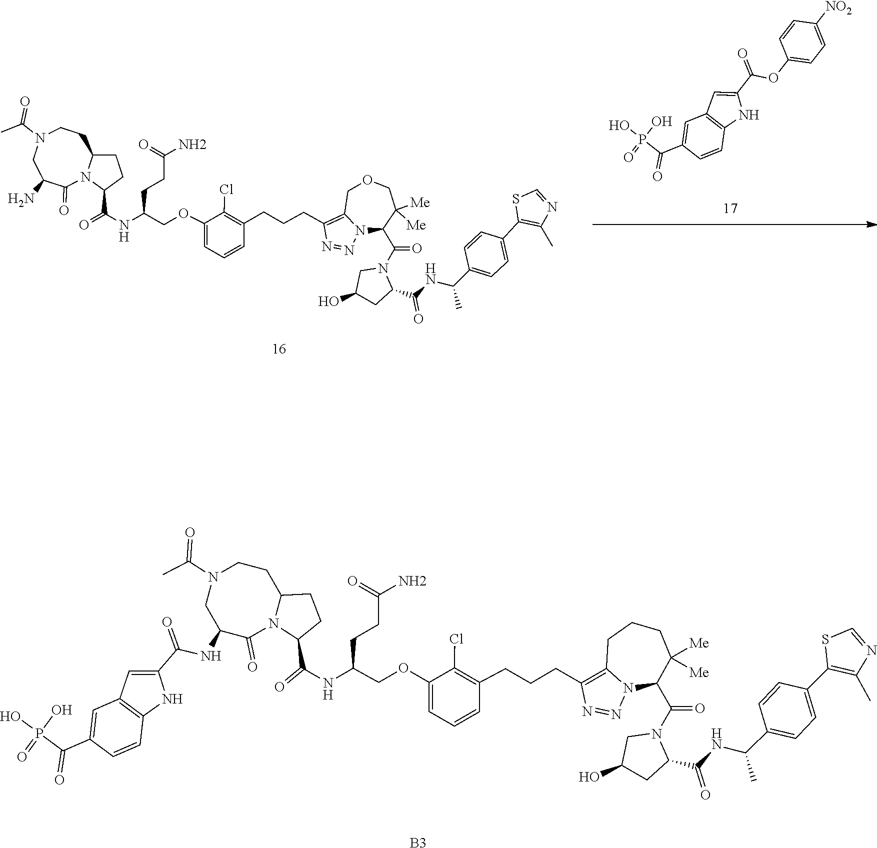

DIEA (0.042 mmol, 3 equiv.) was added to the mixture of 17 (0.017 mmol, 1.2 equiv.), compound 16 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B3. UPLC-MS calculated [M+2H]²⁺: 655.2, found: 655.4. Retention time: 3.6 min.

### Compound B4. (2-(((5S,8S,10aR)-3-acetyl-8-(((S)-5-amino-1-(2-chloro-3-(5-((S)-3-cyclopropyl-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-7,8-dihydro-4H-[1,2,3]triazolo[1,5-a][1,4]diazepin-5 (6H)-yl)-5-oxopentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

### Part I: Synthesis of Intermediate-901.

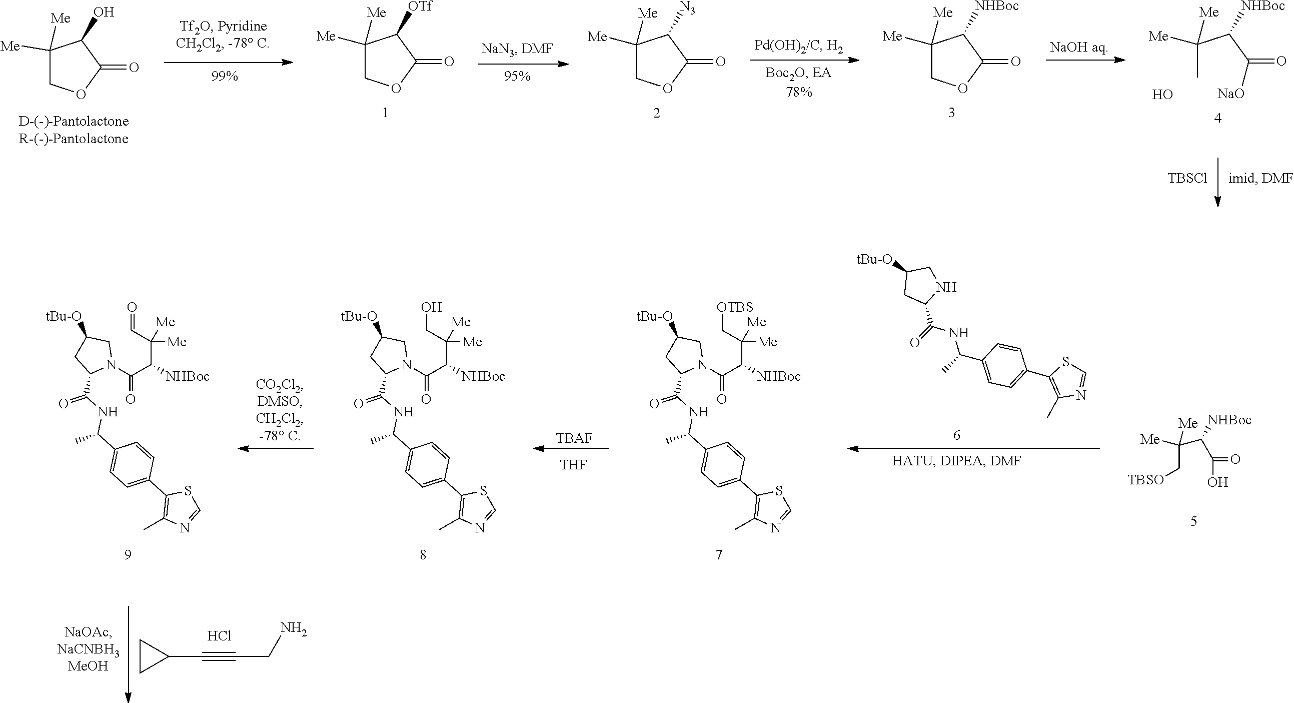

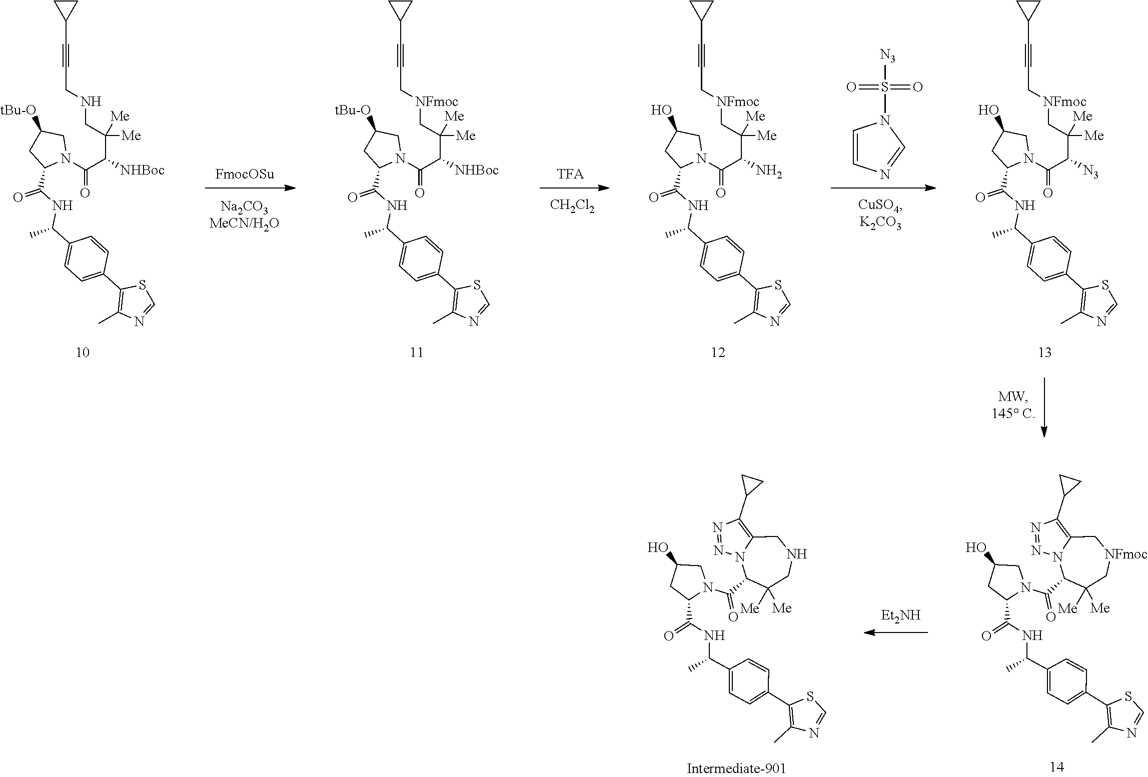

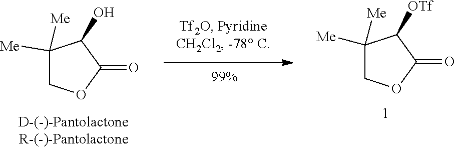 [SMILES: CC1(C)COC(=O)[C@@H]1O ; CC1(C)COC(=O)[C@@H]1OS(=O)(=O)C(F)(F)F]

To a solution of (R)-(−)-Pantolactone (10 g, 1 equiv.) in 4 mL of dry dichloromethane was added pyridine (7.8 mL, 1.25 equiv.). The resulting solution was cooled to −78° C. Triflic anhydride (13.9 mL, 1.1 equiv.) was added dropwise via a syringe over 10 min. The reaction mixture was stirred at −78° C. for 30 min and then 1 h at room temperature. The reaction mixture was partitioned between 200 mL of diethyl ether and 100 mL of 20% HCl. The organic layer was washed once with 50 mL of saturated NaHCO₃, followed by 50 mL of brine. It was dried over MgSO₄ and concentrated in vacuum to afford 20 g 1 (99%), which was used in subsequent step without further purification. Rf₁=0.5, hexane/ethyl acetate=7:3.

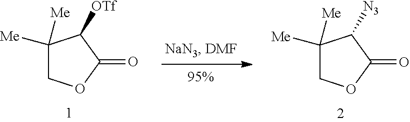 [SMILES: CC1(C)COC(=O)[C@@H]1OS(=O)(=O)C(F)(F)F ; CC1(C)COC(=O)[C@H]1N=[N+]=[N-]]

The crude product 1 (20 g, 1 equiv.) was dissolved in 50 mL of DMF. To the resulting solution was added NaN₃ (5.7 g, 1.15 equiv.). The mixture was stirred at room temperature for 2 h. The reaction mixture was poured into water (200 mL). The aqueous mixture was extracted twice with 100 mL of diethyl ether. The combined organic extract was washed once with brine (50 mL). The organic layer was dried over MgSO₄. Concentrated in vacuum and purified by silica gel flash chromatography to afford 11.3 g white powder 2 (95%). Rf₂=0.26, hexane/ethyl acetate=8:2

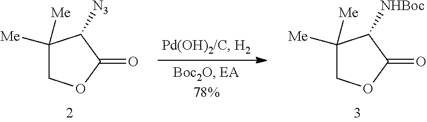 [SMILES: CC1(C)COC(=O)[C@H]1N=[N+]=[N-] ; CC(C)(C)OC(=O)N[C@@H]1C(=O)OCC1(C)C]

To a solution of 2 (2 g, 1 equiv.) in ethyl acetate (70 mL). Boc₂O (5.6 g, 2 equiv.) and Pd(OH)₂/C (0.2 g) were added into the flask. Hydrogenation with H₂ balloon for 24 h. Rf₂=0.71, Rf₃=0.43 Dichloromethane. Pd(OH)₂/C was filtered by Cilite. Concentrated in vacuum and purified by silica gel flash chromatography to afford 2.5 g white powder 3 (84.6%). UPLC-MS: calculated [M+Na]⁺: 252.12, found: 252.13.

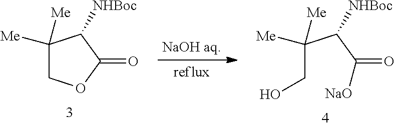

To a solution of 3 (150 mg, 1 equiv.) in NaOH aq. (5.5 mL, C_{Na}OH=0.125 mol/L, 1.05 equiv). The mixture was heated at 50° C. overnight. Once the reaction was detected be done by HPLC-MS, remove the water by freeze drying in vacuum to afford 4, which was used in subsequent step without further purification. UPLC-MS: calculated [M+H]⁺: 248.15, found: 247.99.

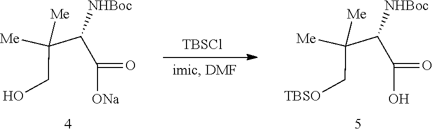

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

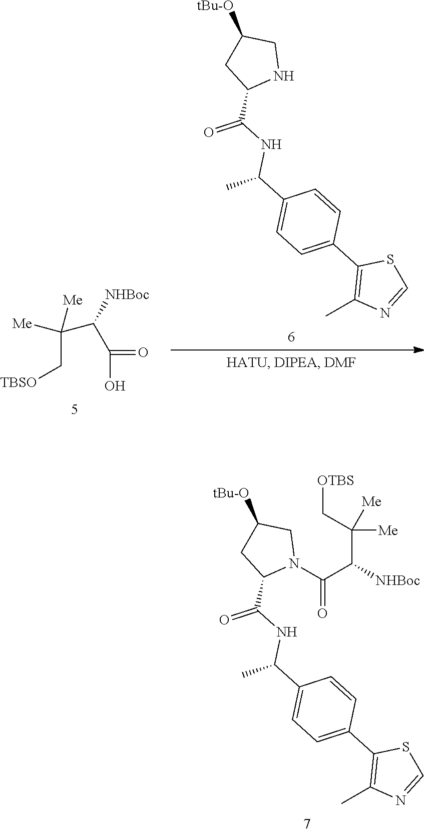

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 731.4, found: 731.6.

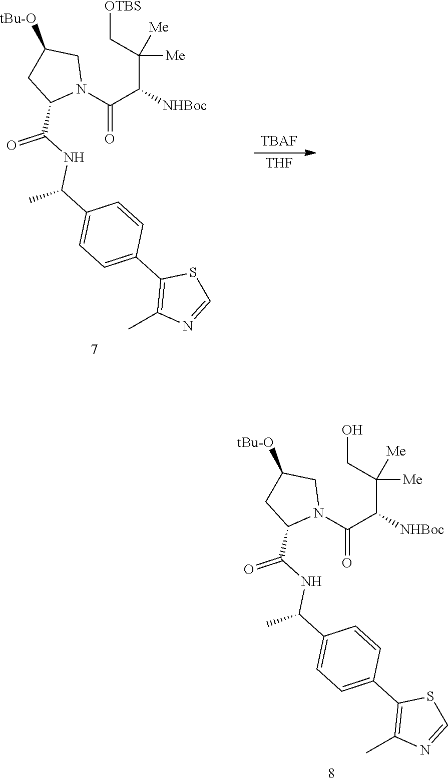

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+H]⁺: 617.3, found: 617.6.

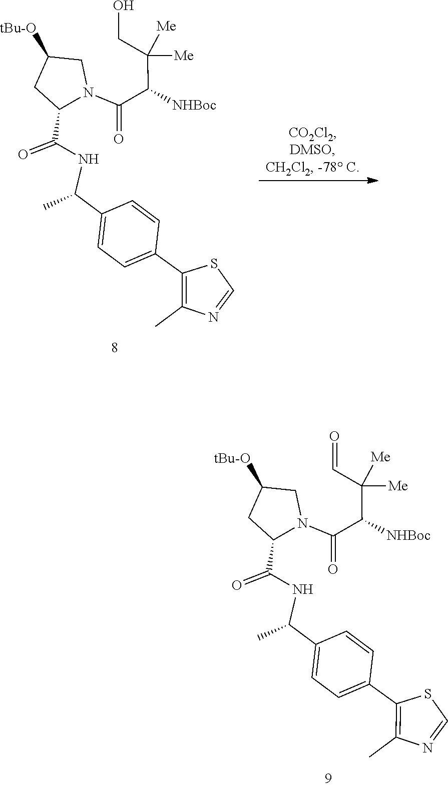

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 615.3, found: 615.7.

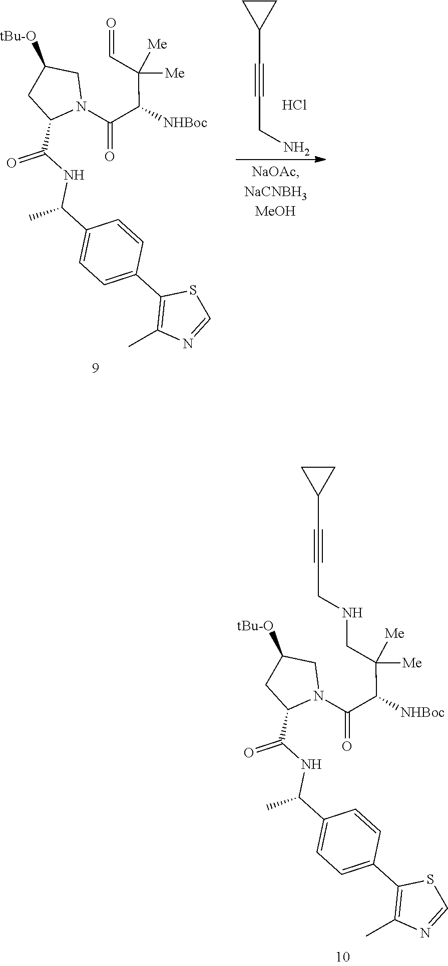

To a solution of 9 (1 equiv.) in MeOH. 3-cyclopropylprop-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 694.4, found: 694.5.

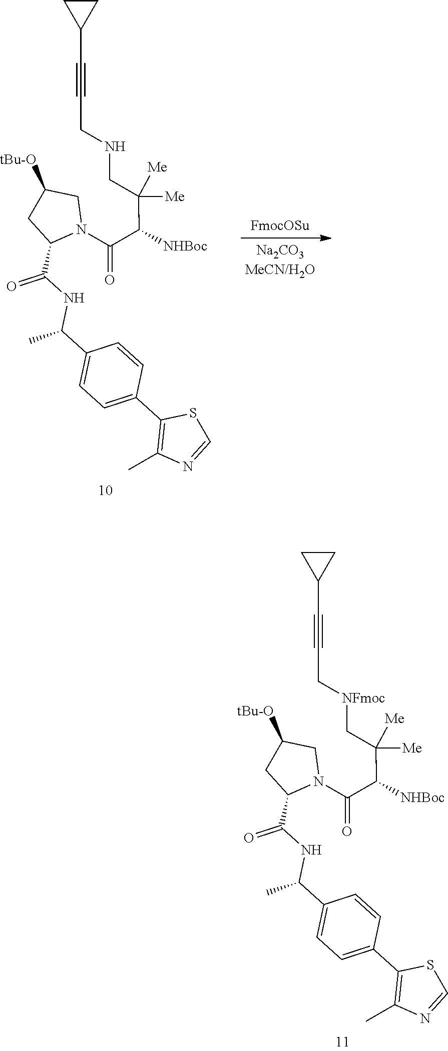

To a solution of 10 (1 equiv.) in MeCN/H₂O (2:1). Na₂CO₃ (3 equiv.) and FmocOSu (2.0 equiv.) were added. The reaction was stirred for 6 hours. Then NaBCNH₃ (2 equiv.) was added into 8 portions. The mixture was stirred for 2 h. The ethyl acetate was added and water phase was washed twice by ethyl acetate. The organic phase was combined, dried, concentrated by vacuum and purified by silica column chromatography to afford 10. UPLC-MS: calculated [M+H]⁺: 916.5, found: 916.8.

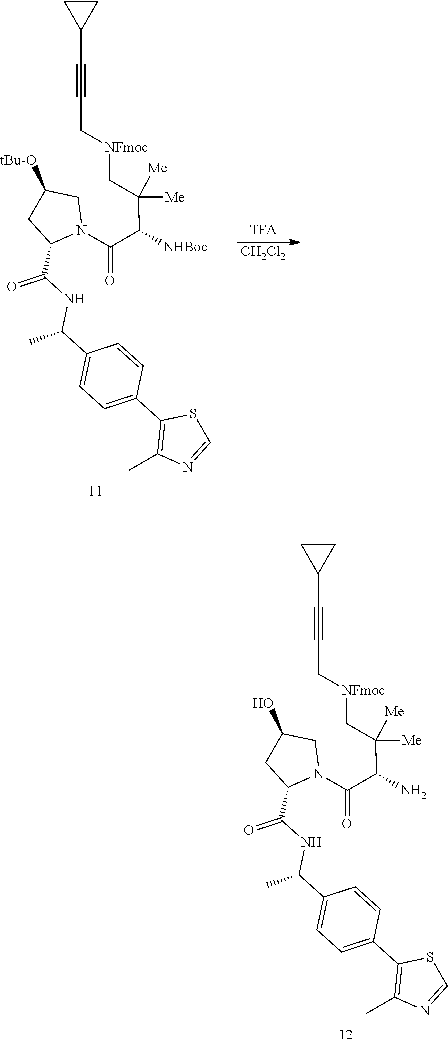

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 760.4, found: 760.3.

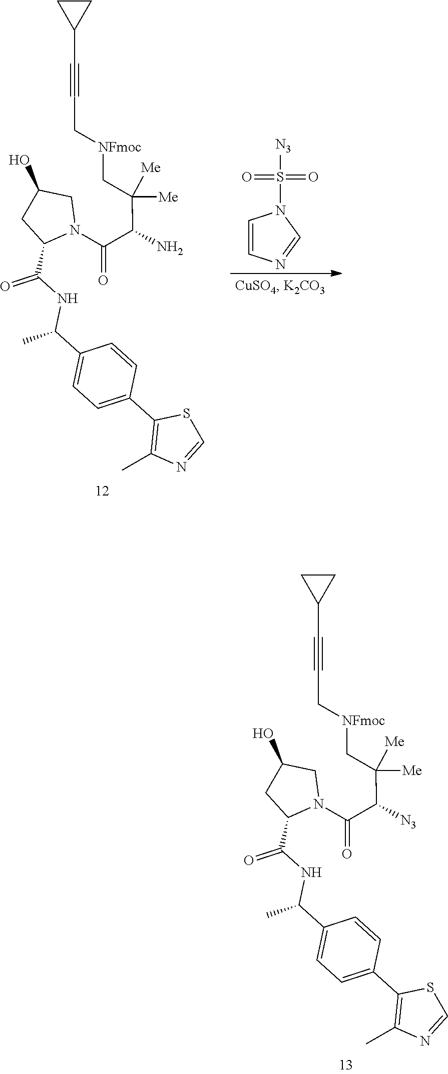

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 786.3, found: 786.5.

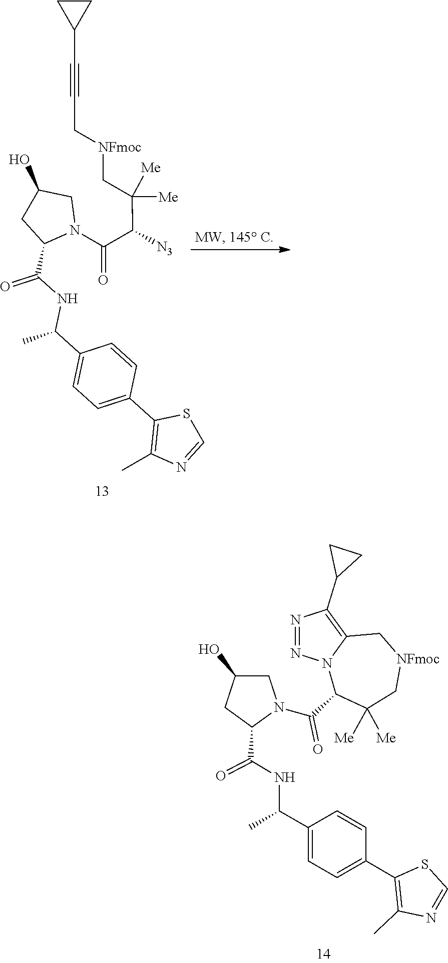

To a solution of 13 (1 equiv.) in 1,2-DCB (c₁₃=0.01 mol/L). Microwave the reaction at 145° C. for 16 h. The solvent was removed in vacuum for next step without further purification. UPLC-MS: calculated [M+H]⁺: 786.3, found: 786.4.

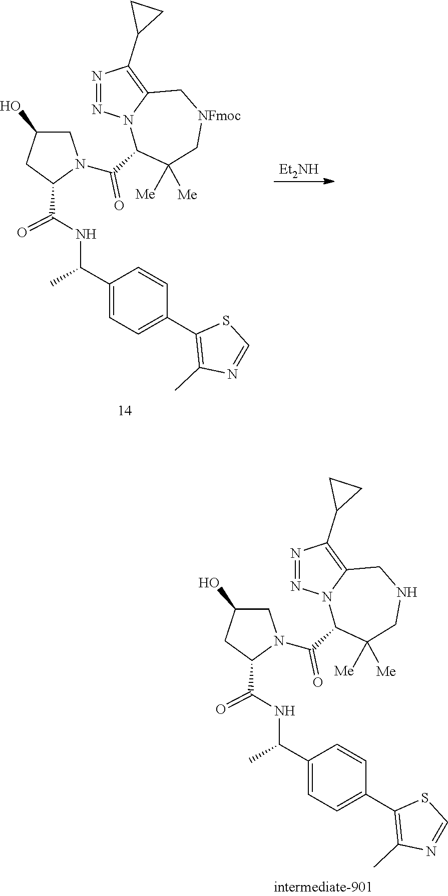

To a solution of 14 (1 equiv.) MeCN. Then diethylamine (5 equiv.) was added. The mixture was stirred for 3 hours. The mixture was detected by UPLC-MS and purified by HPLC to afford intermediate-901. UPLC-MS: calculated [M+H]+:564.3, found: 564.3.

### Part II. Synthesis of Compound B4

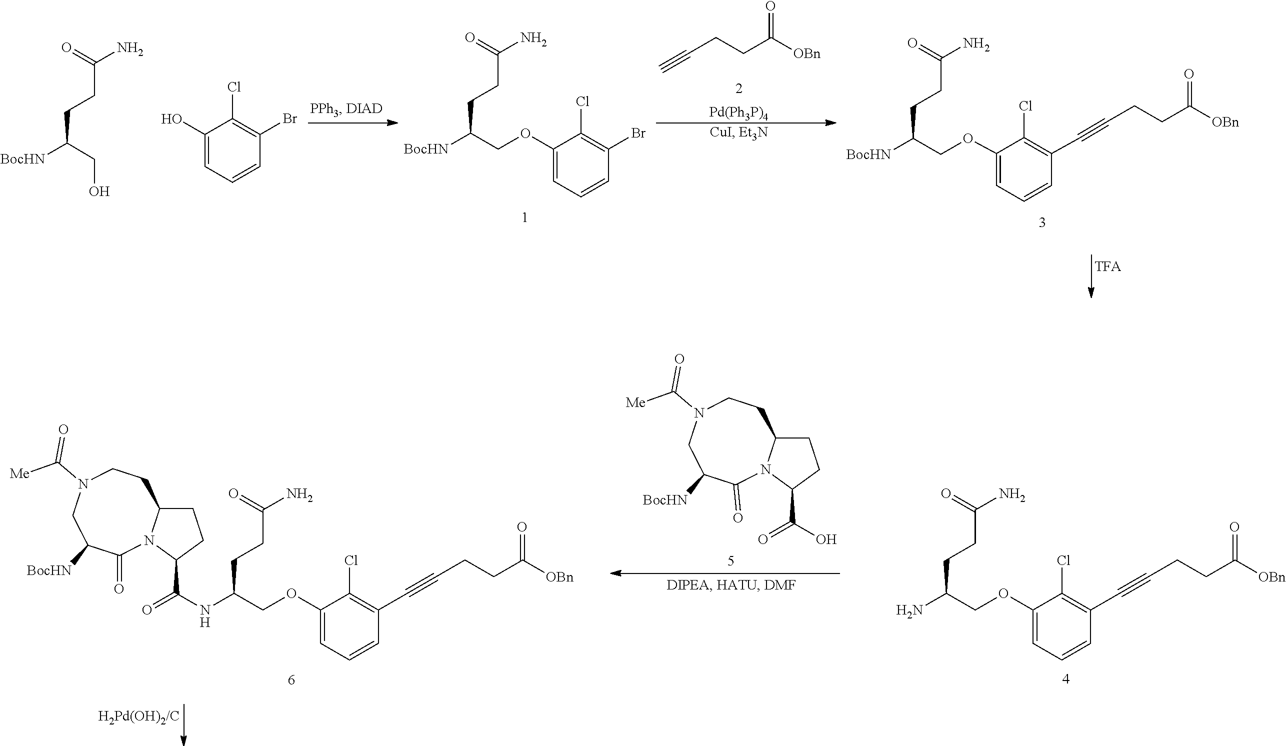

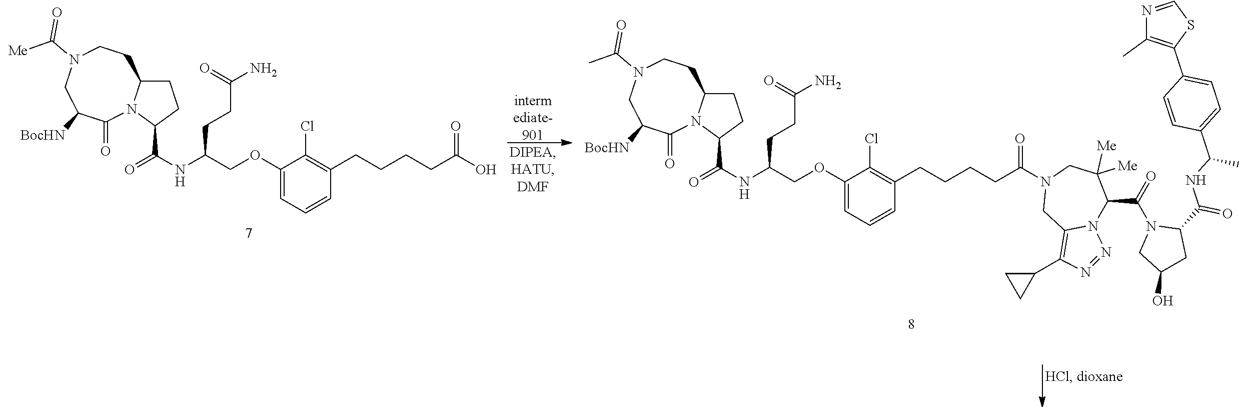

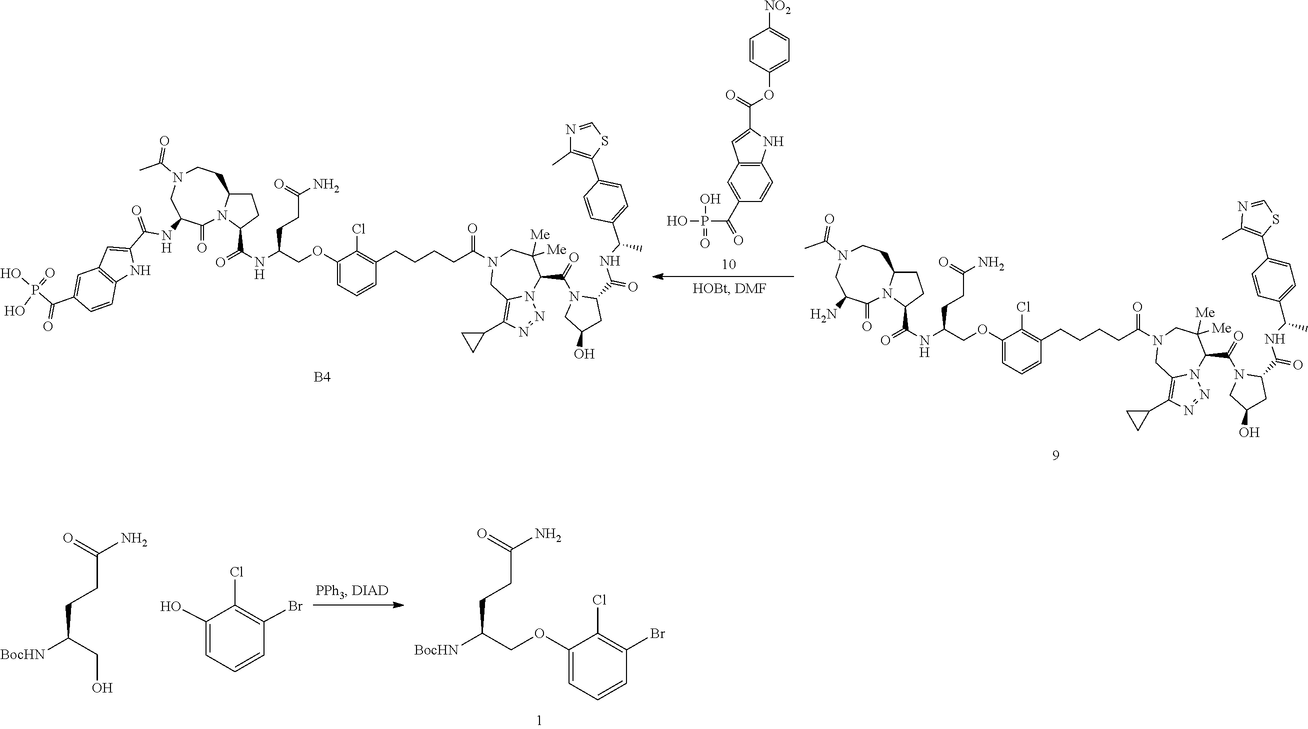

To a solution of 3-bromo-2-chlorophenol (1 equiv.) and Boc-glutaminol (1 equiv.) in THF. PPh₃ (2 equiv) was added into flask. DIAD (2 equiv.) was added in 4 portions. The reaction was stirred for 30 min. The solvent was removed in vacuum and purified by silica column chromatography to afford 1. UPLC-MS: calculated [M+H]⁺: 421.0, found: 421.2.

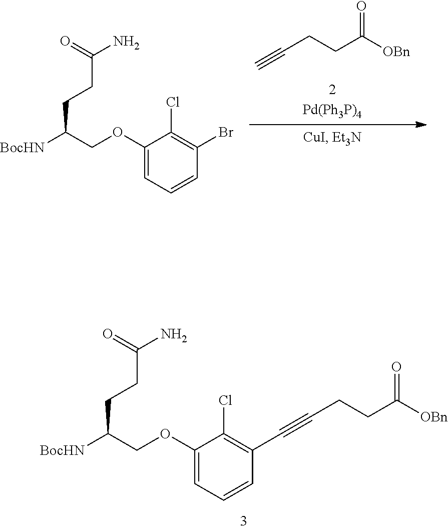 [SMILES: CC(C)(C)OC(=O)N[C@@H](CCC(N)=O)COc1cccc(Br)c1Cl ; C#CCCC(=O)OCc1ccccc1 ; CC(C)(C)OC(=O)N[C@@H](CCC(N)=O)COc1cccc(C#CCCC(=O)OCc2ccccc2)c1Cl]

To a solution of 1 (1 equiv.) in DMSO(c₁=0.15 mol/L) was added 2 (4 equiv.), Et₃N (1/2 volume of DMSO), CuI (0.1 equiv.) and Pd(PPh₃)₄ (0.1 equiv.) at room temperature under nitrogen atmosphere. The reaction mixture was stirred for 16 hours at 90° C. under nitrogen atmosphere. The resulting mixture cooled down to room temperature and concentrated under reduced pressure. The residue was purified by HPLC to afford 3. UPLC-MS: calculated [M+H]⁺: 529.2, found: 529.3.

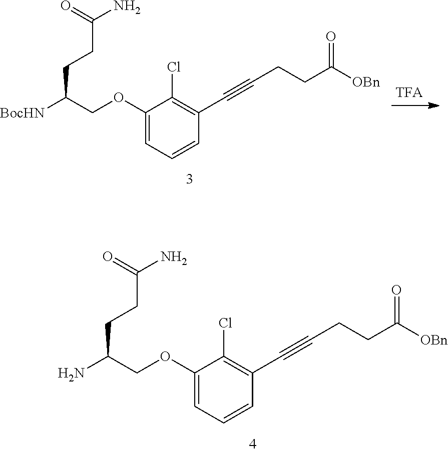 [SMILES: CC(C)(C)OC(=O)N[C@@H](CCC(N)=O)COc1cccc(C#CCCC(=O)OCc2ccccc2)c1Cl ; NC(=O)CC[C@H](N)COc1cccc(C#CCCC(=O)OCc2ccccc2)c1Cl]

3 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 429.2, found: 429.1.

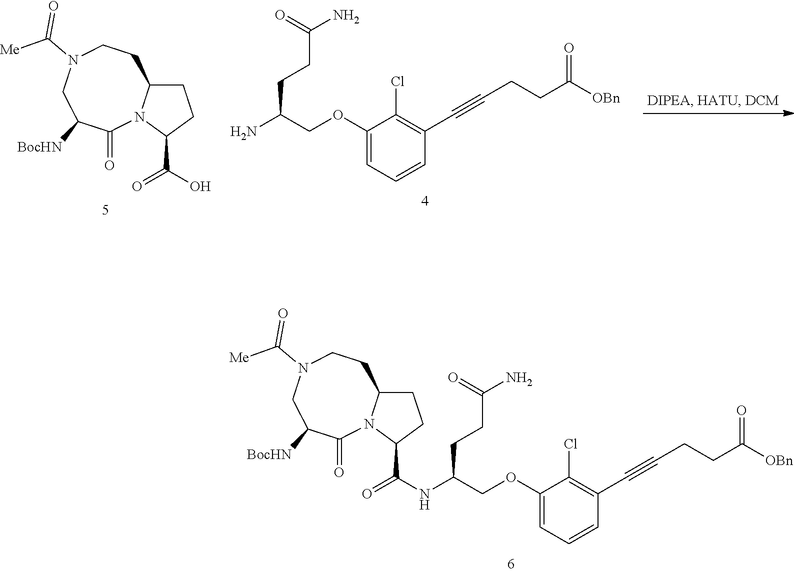 [SMILES: CC(=O)N1CC[C@H]2CC[C@@H](C(=O)O)N2C(=O)[C@@H](NC(=O)OC(C)(C)C)C1 ; NC(=O)CC[C@H](N)COc1cccc(C#CCCC(=O)OCc2ccccc2)c1Cl ; CC(=O)N1CC[C@H]2CC[C@@H](C(=O)N[C@@H](CCC(N)=O)COc3cccc(C#CCCC(=O)OCc4ccccc4)c3Cl)N2C(=O)[C@@H](NC(=O)OC(C)(C)C)C1]

To a solution of 5 (1 equiv.) and 4 (1 equiv.) in DCM. Then DIPEA (5 equiv.) and HATU (1 equiv.) were added. The mixture was stirred for 10 min. The mixture was detected by UPLC-MS and purified by silica column chromatography to afford 6. UPLC-MS: calculated [M+H]⁺: 780.3, found: 780.3.

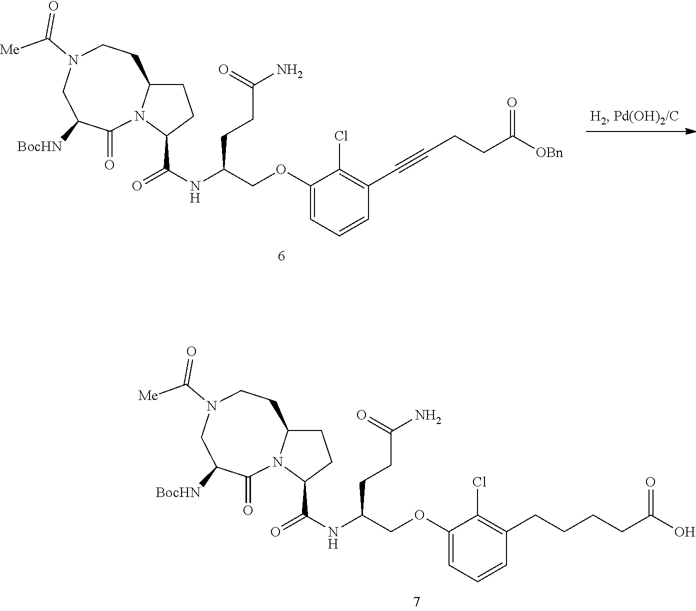 [SMILES: CC(=O)N1CC[C@H]2CC[C@@H](C(=O)N[C@@H](CCC(N)=O)COc3cccc(C#CCCC(=O)OCc4ccccc4)c3Cl)N2C(=O)[C@@H](NC(=O)OC(C)(C)C)C1 ; CC(=O)N1CC[C@H]2CC[C@@H](C(=O)N[C@@H](CCC(N)=O)COc3cccc(CCCCC(=O)O)c3Cl)N2C(=O)[C@@H](NC(=O)OC(C)(C)C)C1]

To a solution of 6 (1 equiv.) in MeOH. Pd(OH)₂/C (10% wt) was added. The mixture was stirred under H2 atmosphere overnight. The mixture was filtered by Celite and concentrated in vacuum to afford 7 for next step without any purification. UPLC-MS: calculated [M+H]⁺: 694.3, found: 694.4.

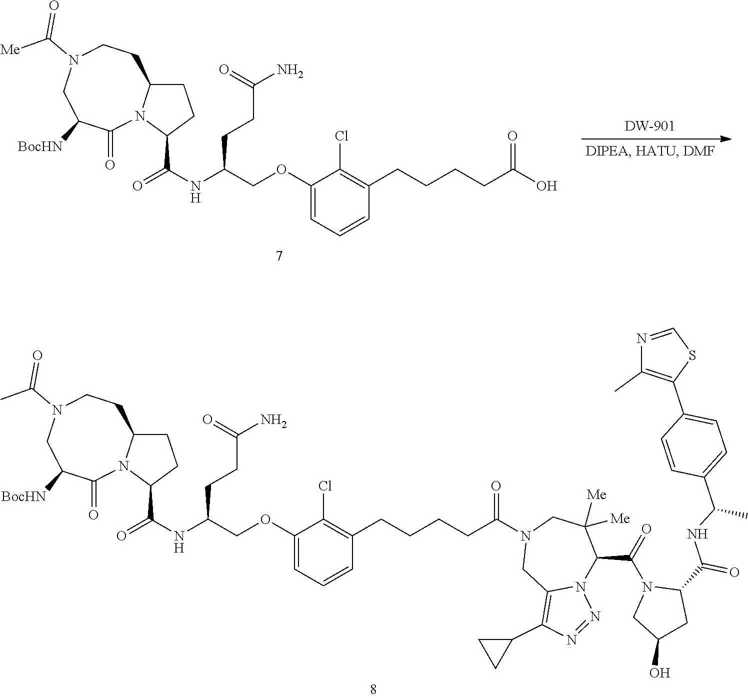 [SMILES: CC(=O)N1CC[C@H]2CC[C@@H](C(=O)N[C@@H](CCC(N)=O)COc3cccc(CCCCC(=O)O)c3Cl)N2C(=O)[C@@H](NC(=O)OC(C)(C)C)C1 ; CC(=O)N1CC[C@H]2CC[C@@H](C(=O)N[C@@H](CCC(N)=O)COc3cccc(CCCCC(=O)N4Cc5c(C6CC6)nnn5[C@H](C(=O)N5C[C@H](O)C[C@H]5C(=O)N[C@@H](C)c5ccc(-c6scnc6C)cc5)C(C)(C)C4)c3Cl)N2C(=O)[C@@H](NC(=O)OC(C)(C)C)C1]

To a solution of 7 (1 equiv.) and Intermediate-901 (1 equiv.) in DMF. Then DIPEA (5 equiv.) and HATU (1 equiv.) were added. The mixture was stirred for 10 min. The mixture was detected by UPLC-MS and purified by HPLC to afford 8. UPLC-MS: calculated [M+2H]/2⁺:621.0, found: 620.7.

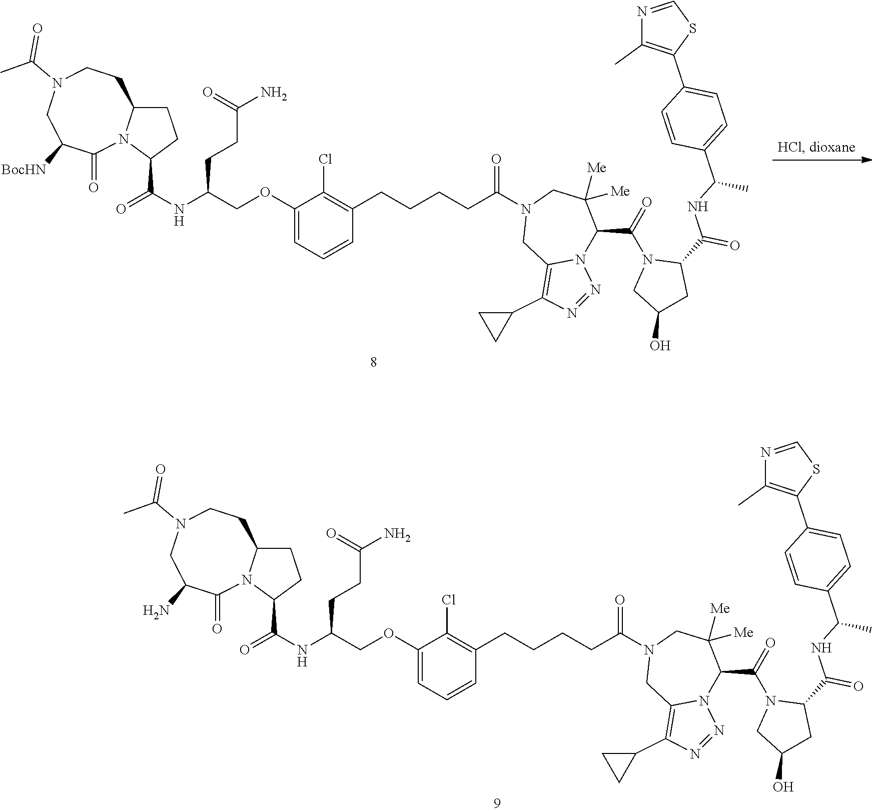

8 (1 equiv.) was dissolved in CH₂Cl₂, HCl (4M in dioxane, 5 equiv.) was added. The reaction was stirred for one hours. Solvent was removed in vacuum for next step without further purification. UPLC-MS: calculated [M+H]⁺: 1139.5, found: 1139.8.

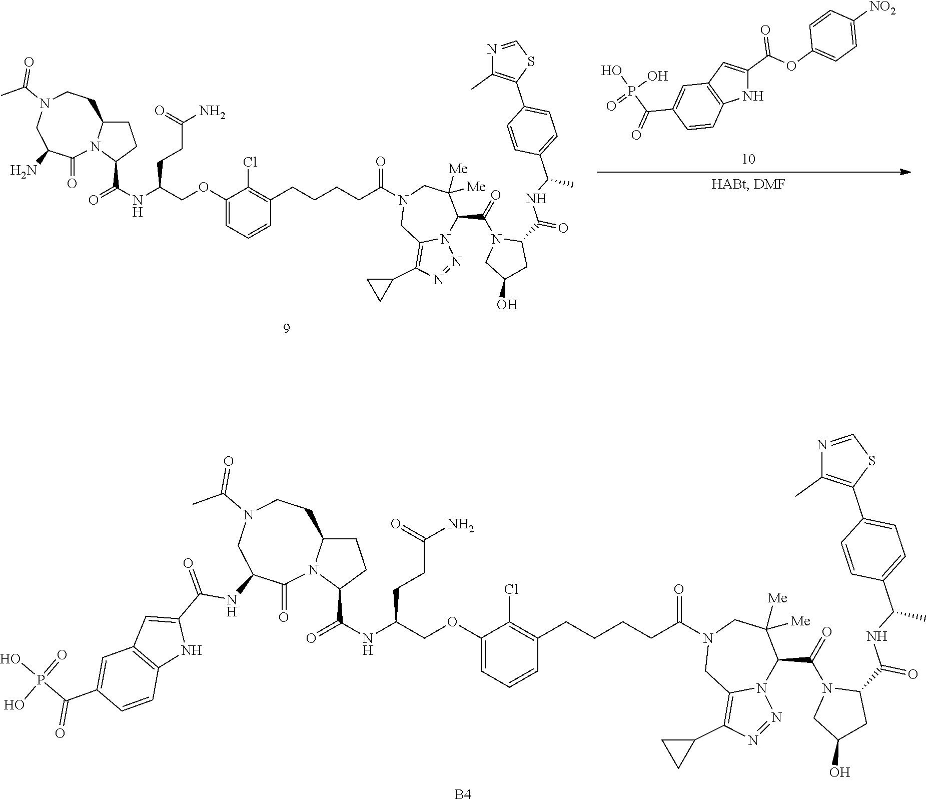 [SMILES: CC(=O)N1CC[C@H]2CC[C@@H](C(=O)N[C@@H](CCC(N)=O)COc3cccc(CCCCC(=O)N4Cc5c(C6CC6)nnn5[C@H](C(=O)N5C[C@H](O)C[C@H]5C(=O)N[C@@H](C)c5ccc(-c6scnc6C)cc5)C(C)(C)C4)c3Cl)N2C(=O)[C@@H](N)C1 ; O=C(Oc1ccc([N+](=O)[O-])cc1)c1cc2cc(C(=O)P(=O)(O)O)ccc2[nH]1 ; CC(=O)N1CC[C@H]2CC[C@@H](C(=O)N[C@@H](CCC(N)=O)COc3cccc(CCCCC(=O)N4Cc5c(C6CC6)nnn5[C@H](C(=O)N5C[C@H](O)C[C@H]5C(=O)N[C@@H](C)c5ccc(-c6scnc6C)cc5)C(C)(C)C4)c3Cl)N2C(=O)[C@@H](NC(=O)c2cc3cc(C(=O)P(=O)(O)O)ccc3[nH]2)C1 ; "B4"]

9 (1 equiv.) was dissolved in DMF. Then 10 (1 equiv.) and HOBt (1 equiv.) was added. The reaction was stirred for one hour. Then purified by HPLC to afford B4. UPLC-MS: calculated [M+2H]/2⁺:695.8, found: 695.9.

### Compound B5. (2-(((5S,8S,10aR)-3-acetyl-8-(((S)-1-(3-(5-((R)-3-((2R,4S)-1-((R)-5-acetyl-3-cyclopropyl-7,7-dimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanamido)pentyl)-2-chlorophenoxy)-5-amino-5-oxopentan-2-yl)carbamoyl)-6-oxodecahydropyrrolo[1,2-a][1,5]diazocin-5-yl)carbamoyl)-1H-indole-5-carbonyl)phosphonic acid

### Compound B6. (2-(((5S,8S,10aR)-3-acetyl-8-(((S)-1-(3-(5-((R)-3-((2R,4S)-1-((R)-5-acetyl-3,7,7-trimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanamido)pentyl)-2-chlorophenoxy)-5-amino-5-oxopentan-2-yl)carbamoyl)-6-oxodecahydropyrrolo[1,2-a][1,5]diazocin-5-yl)carbamoyl)-1H-indole-5-carbonyl)phosphonic acid

### Compound B12. (2-(((5S,8S,10aR)-3-acetyl-8-(((S)-5-amino-1-(2-chloro-3-(5-((R)-3-((2R,4S)-1-((R)-3-cyclopropyl-5,7,7-trimethyl-5,6,7,8-tetrahydro-4H-[1,2,3]triazolo[1,5-a][1,4]diazepine-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

### Compound B13. (2-(((5S,8S,10aR)-3-acetyl-8-(((S)-1-(3-(5-((R)-3-((2R,4S)-1-((S)-6-acetyl-3,8,8-trimethyl-4,5,6,7,8,9-hexahydro-[1,2,3]triazolo[1,5-a][1,5]diazocine-9-carbonyl)-4-hydroxypyrrolidine-2-carboxamido)-3-(4-(4-methylthiazol-5-yl)phenyl)propanamido)pentyl)-2-chlorophenoxy)-5-amino-5-oxopentan-2-yl)carbamoyl)-6-oxodecahydropyrrolo[1,2-a][1,5]diazocin-5-yl)carbamoyl)-1H-indole-5-carbonyl)phosphonic acid

### Part I: Synthesis of Intermediate-930, 937, and 945

| Code | Amine 10 | Structure |
|---|---|---|
| Intermediate-930 | 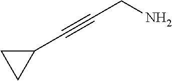 | 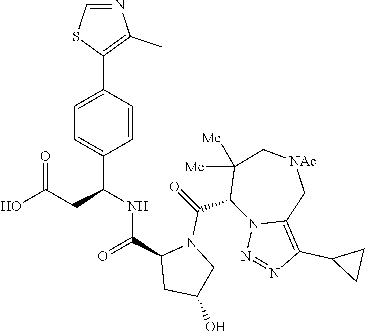 |
| Intermediate-937 | 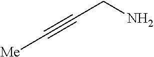 | 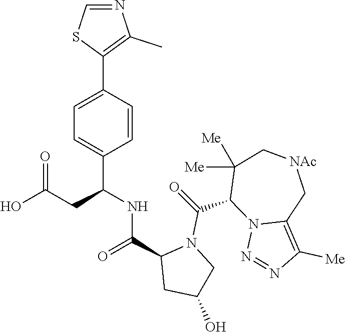 |
| Intermediate-945 | 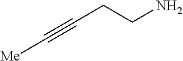 | 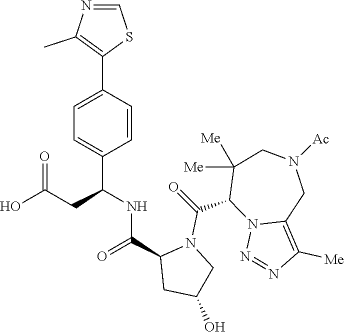 [SMILES: CC(=O)N1Cc2c(C)nnn2[C@H](C(=O)N2C[C@H](O)C[C@H]2C(=O)N[C@@H](CC(=O)O)c2ccc(-c3scnc3C)cc2)C(C)(C)C1] |

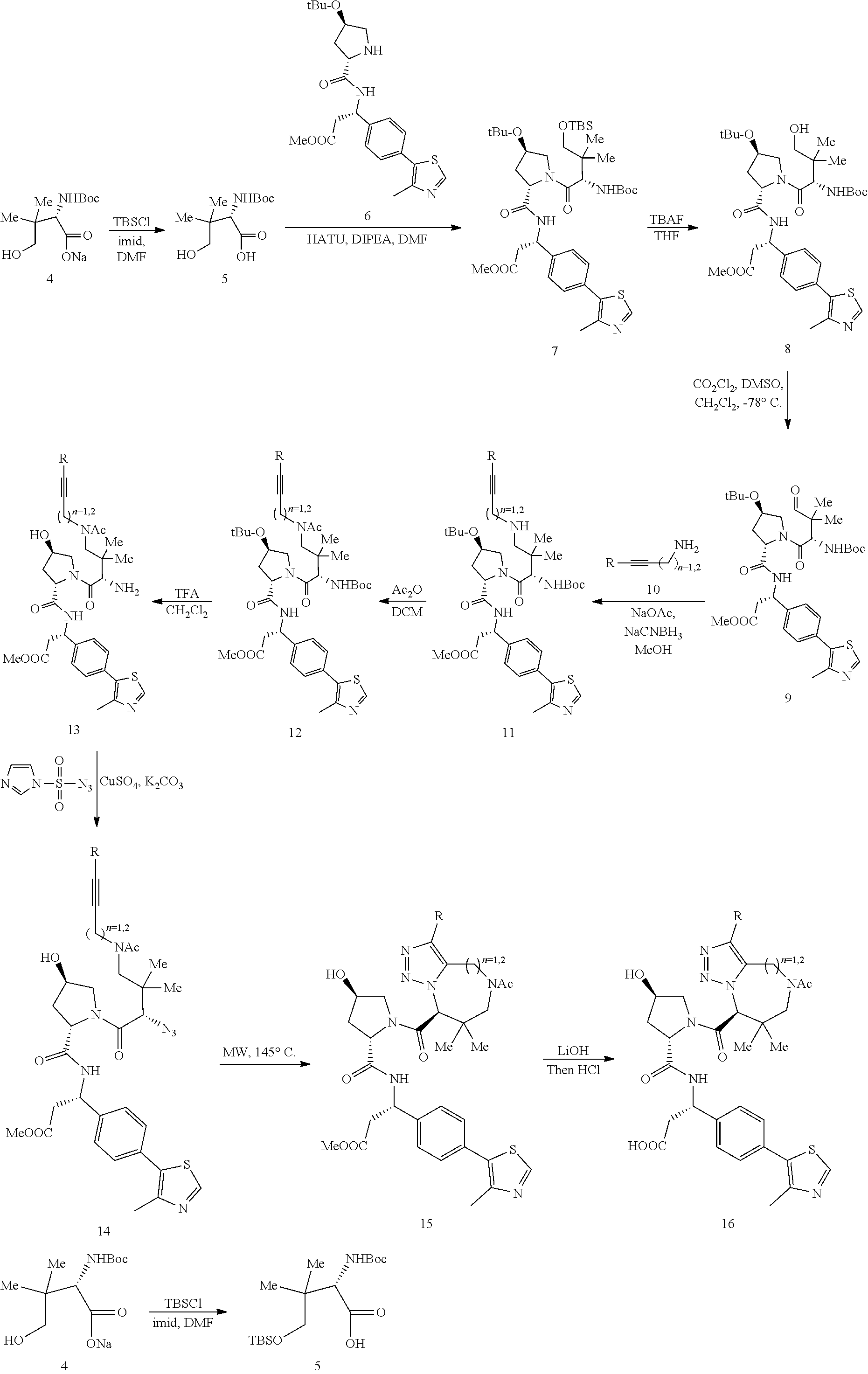

To a solution of 4 (1 equiv.) and imidazole (1.5 equiv.) in DMF (c_{intermediate1}=1 mol/L). Then TBSCl (1.1 equiv.) was added at room temperature. The mixture was stirred for 2 hours. Then the reaction was quenched by water, HCl acid (2M) was added at 0° C. until PH=5. The water phase was separated and washed by ethyl acetate. Combined organic phase and washed with brine, dried, concentrated for next step without further purification. UPLC-MS: calculated [M+H]⁺: 362.2, found: 362.3.

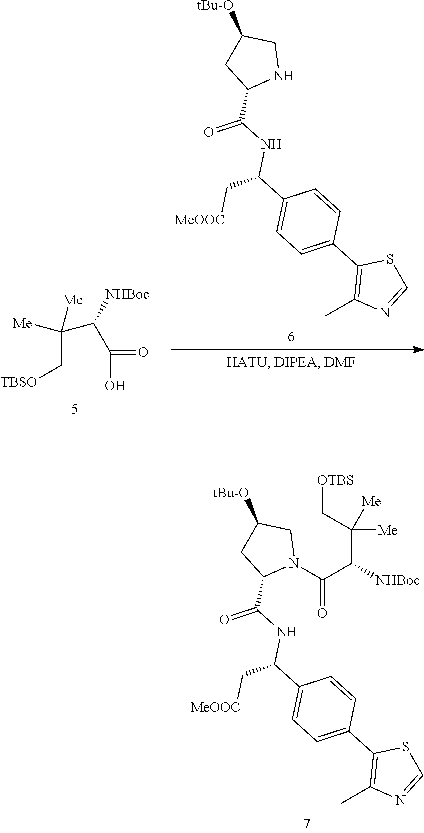

To a solution of 5 (1 equiv.) and 6 (1 equiv.) in DMF. Then DIPEA (5 equiv.) and HATU (1 equiv.) were added. The mixture was stirred for 10 min. The mixture was detected by UPLC-MS and purified by HPLC to afford 7. UPLC-MS: calculated [M+H]⁺: 789.4, found: 788.9.

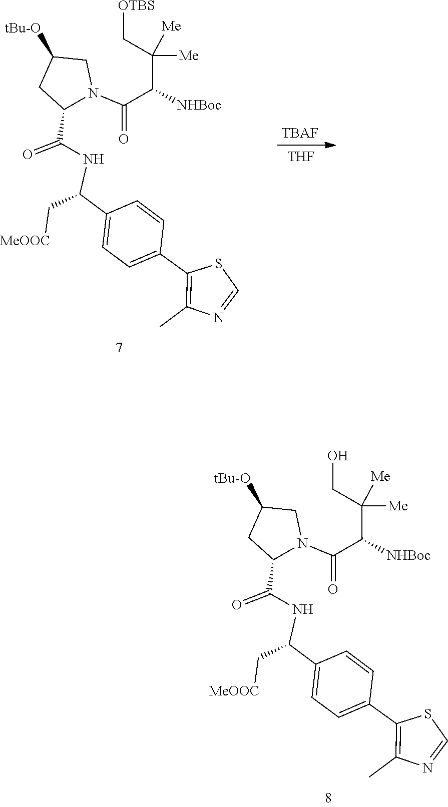

7 (1 equiv.) was dissolved in THF (c₇=0.2 mol/L), TBAF (1 mol/L in THF, 2 equiv.) was added at room temperature. The reaction was stirred for 3 hour. The reaction was quenched by water and the aqueous mixture was extracted twice with ethyl acetate. The combined organic extract was washed once with brine (50 mL). The organic layer was dried over MgSO₄. Concentrated in vacuum and purified by silica gel flash chromatography to afford 8. UPLC-MS: calculated [M+Na]⁺: 697.3, found: 697.0.

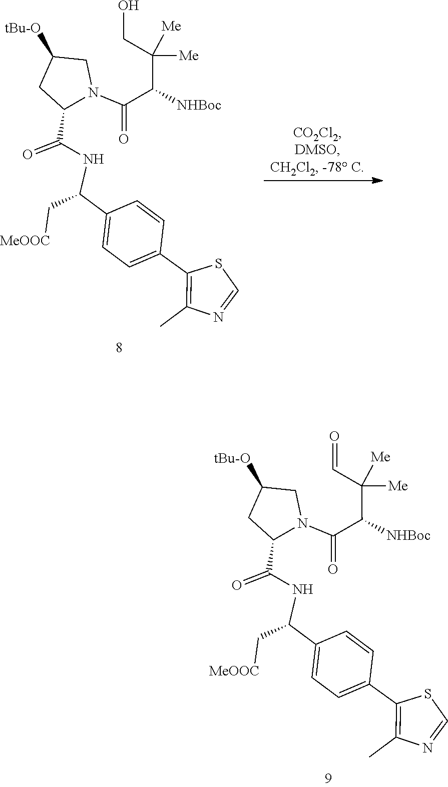

To a solution of oxalyl chloride (2.0 equiv.) in CH₂Cl₂, the mixture was stirred at −78° C. DMSO (2.0 equiv.) was added into the flask. The mixture was stirred for 10 min at −78° C. Then 8 (1 equiv.) in CH₂Cl₂ was added and stirred for 30 min. Et₃N (5.0 equiv.) was added and the flask was removed from −78° C. bath, the reaction was stirred for 30 min. The reaction was quenched by water. The organic phase was separated, washed with brine, dried, concentrated and the residue was purified by silica column chromatography to afford 9 UPLC-MS: calculated [M+H]⁺: 673.3, found: 673.3.

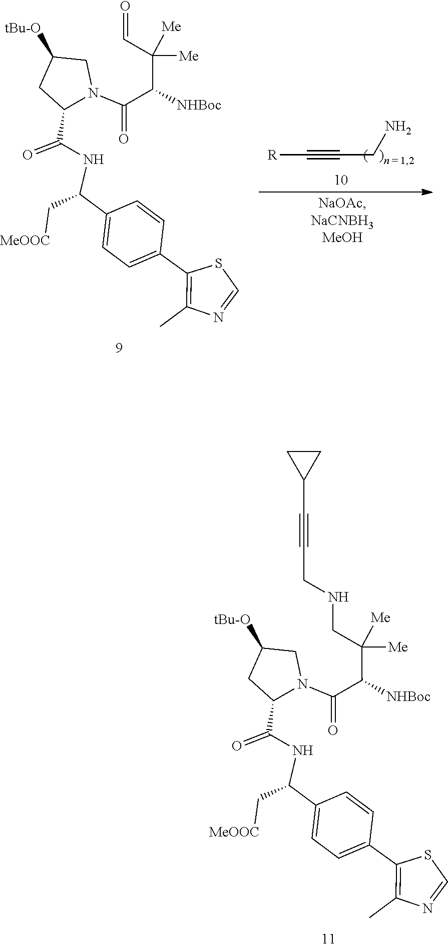

To a solution of 9 (1 equiv.) in MeOH. Amine 10 (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 11.

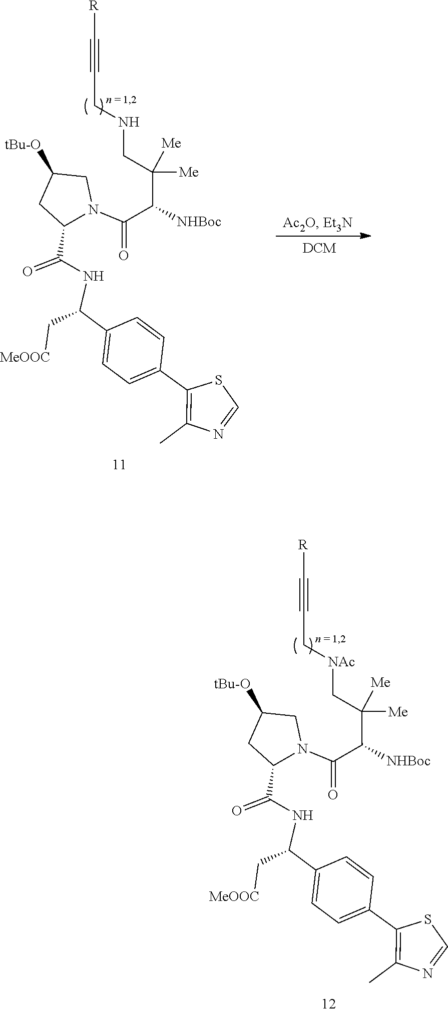

To a solution of 11 (1 equiv.) in DCM. Et₃N (3 equiv.) and Ac₂O (1.2 equiv.) were added. The reaction was stirred for 1 hours. The mixture was concentrated by vacuum and purified by silica column chromatography to afford 12.

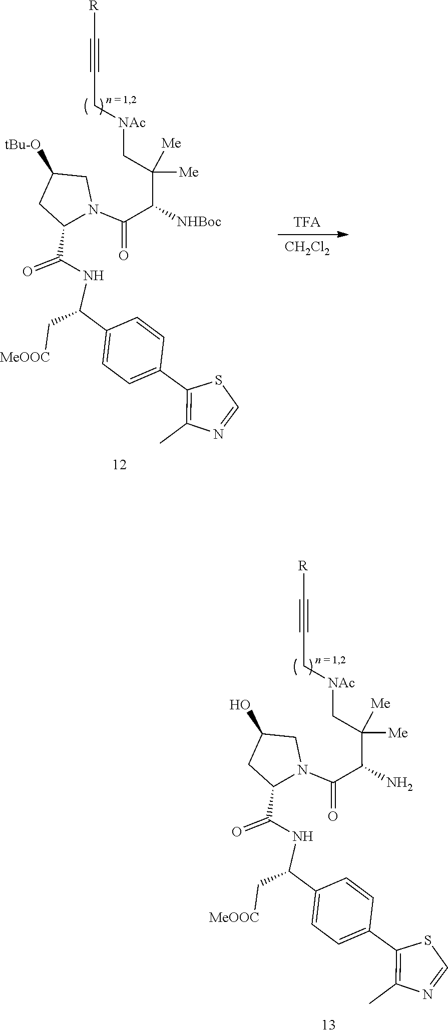

12 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification.

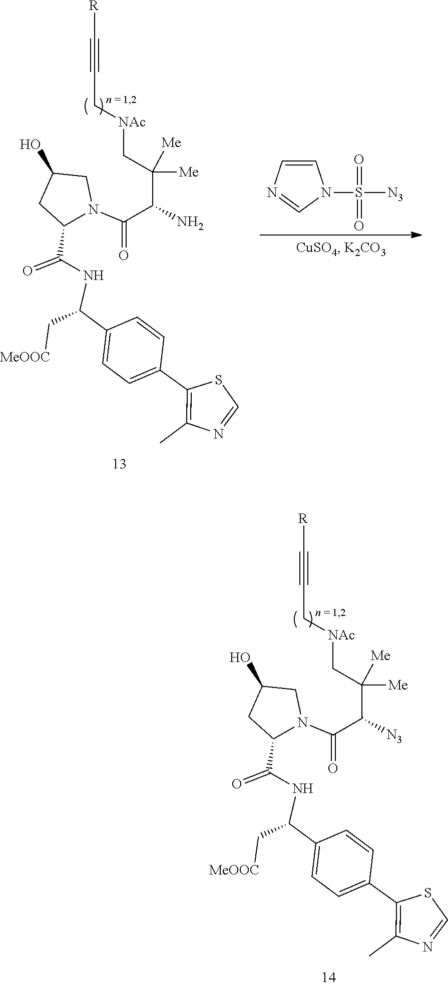

1H-imidazole-1-sulfonyl azide (2 equiv.) was added to a mixture of 13 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 14.

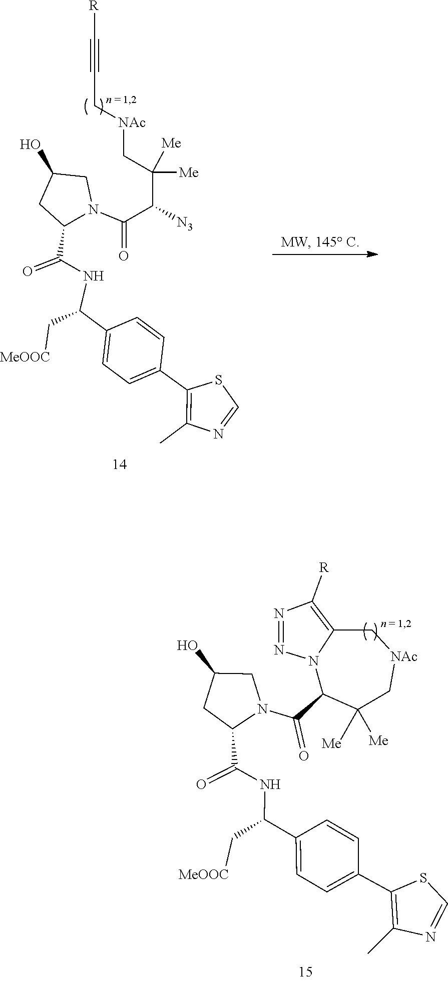

To a solution of 14 (1 equiv.) in 1,2-DCB (c₁₃=0.05 mol/L). Microwave the reaction at 145° C. for 16 h (intermediate-945 needs 180° C. for 30 h). The reaction was purified by silica column chromatography to afford 15.

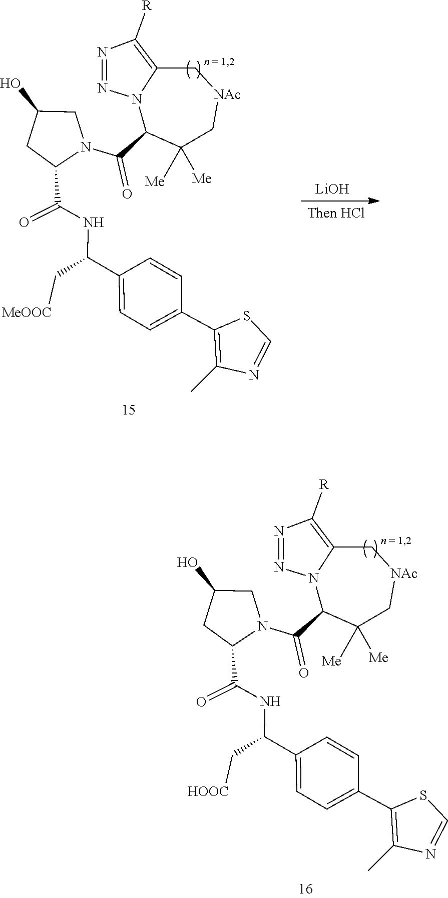

To a solution of 15 (1 equiv.) in MeOH/H₂O (c₁₅=0.1 mol/L, 3:1). LiOH (3 equiv) was added, the mixture was stirred for 3 h. Then the reaction was quenched by HCL (4 mol/L). The reaction was purified by HPLC to afford Intermediate-930, 937, and 945.

### Part II: Synthesis of Intermediate-949

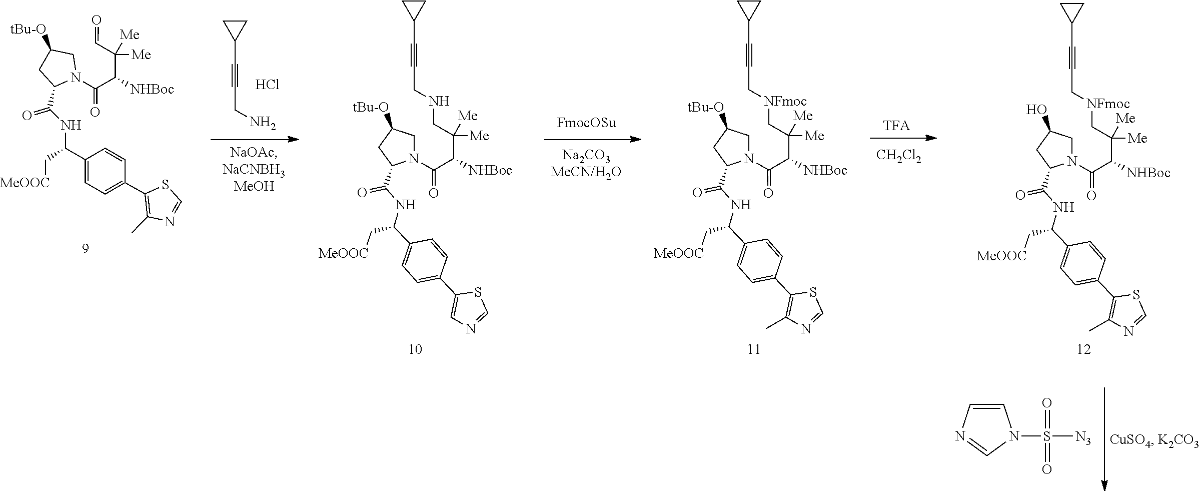

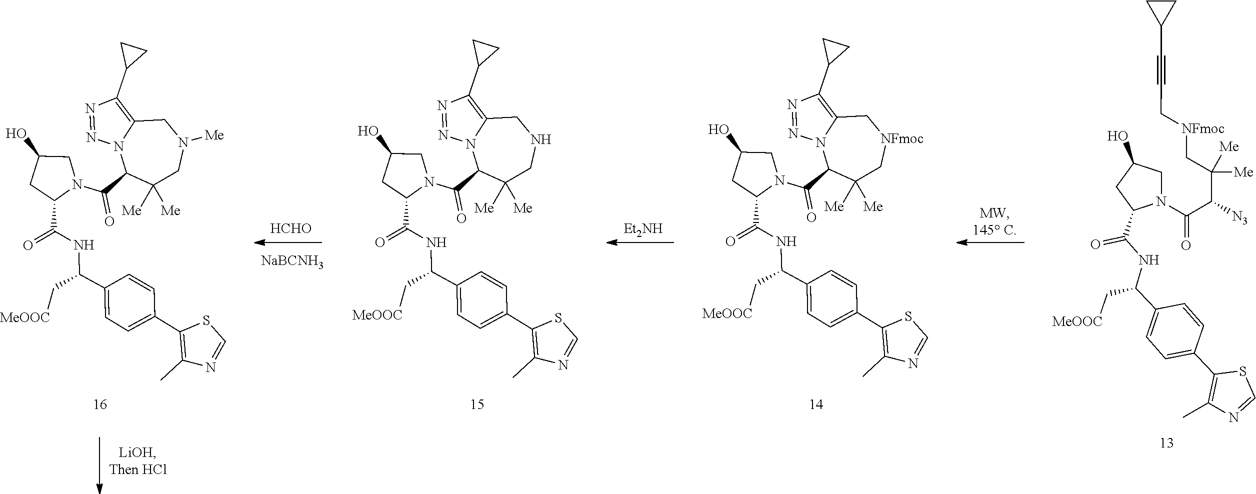

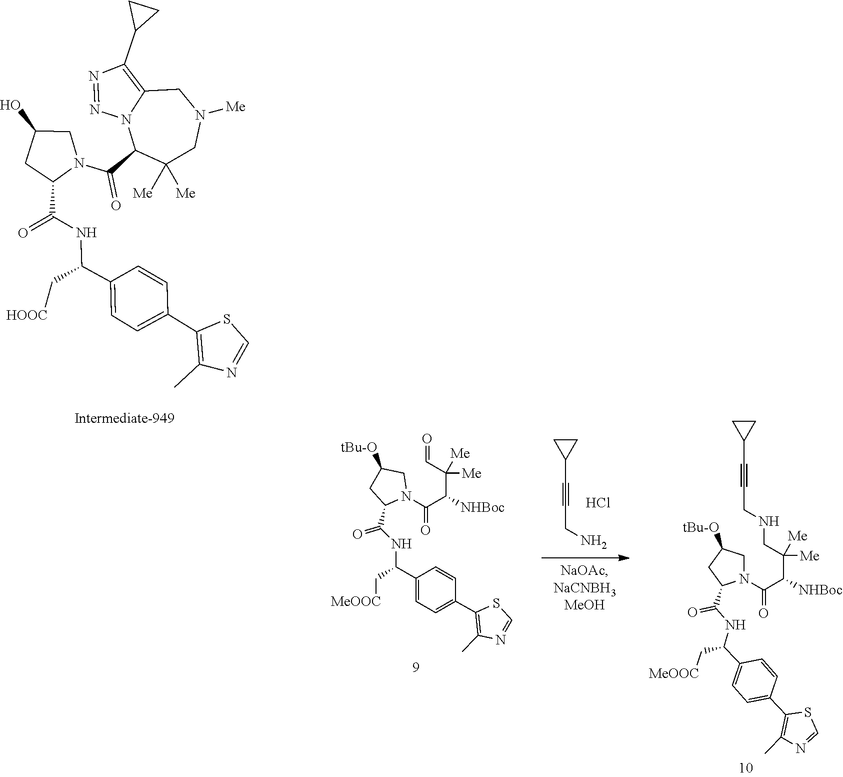

To a solution of 9 (1 equiv.) in MeOH. 3-cyclopropylprop-2-yn-1-amine hydrochloride (1.5 equiv.) and NaOAc (1.5 equiv.) were added. The reaction was stirred for 30 min Then NaBCNH₃ (2 equiv.) was added into 8 portions. The mixture was stirred for 2 h. The solvent was removed by vacuum and purified by silica column chromatography to afford 10. UPLC-MS: calculated [M+H]⁺: 752.4, found: 752.8.

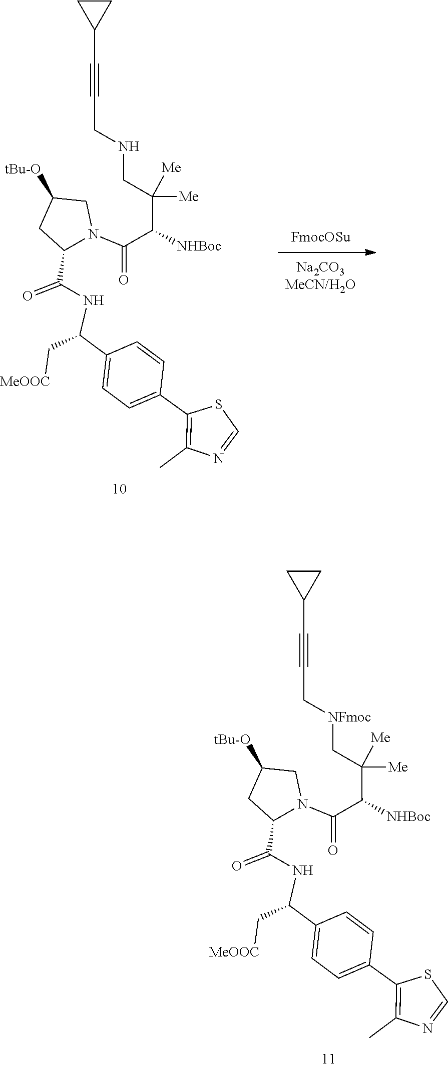

To a solution of 10 (1 equiv.) in MeCN/H₂O (2:1). Na₂CO₃ (3 equiv.) and FmocOSu (2.0 equiv.) were added. The reaction was stirred for 6 hours. The ethyl acetate was added and water phase was washed twice by ethyl acetate. The organic phase was combined, dried, concentrated by vacuum and purified by silica column chromatography to afford 10. UPLC-MS: calculated [M+H]⁺: 974.5, found: 974.6.

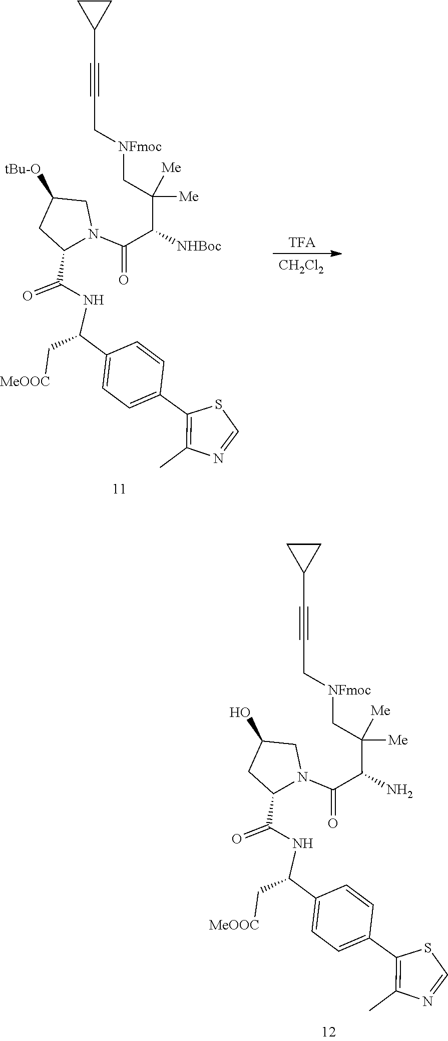

11 was dissolved in CH₂Cl₂ (c₁₁=0.2 mol/L), TFA (1/2 volume of CH₂Cl₂) was added. The reaction was stirred for two hours. Solvent and TFA were removed in vacuum for next step without further purification. UPLC-MS: calculated [M+H]⁺: 818.4, found: 818.2.

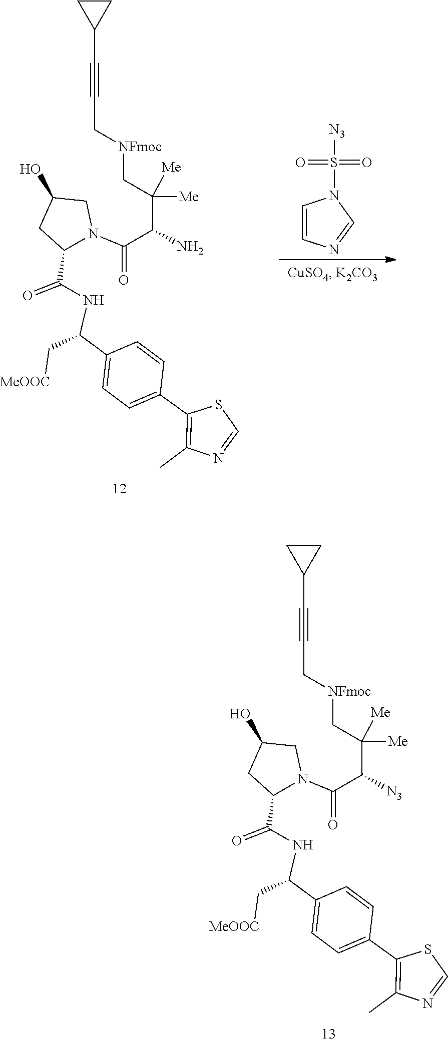

1H-imidazole-1-sulfonyl azide (2 equiv.) was added to a mixture of 12 (1 equiv.), K₂CO₃ (5 equiv.) and CuSO₄ (0.2 equiv.) in MeOH at 25° C. The reaction mixture was stirred at 25° C. for 10 h. After removing the solvent, the organic layer was concentrated, and the residue was purified by reverse phase preparative HPLC to give 13. UPLC-MS calculated [M+H]+: 844.4, found: 844.5.

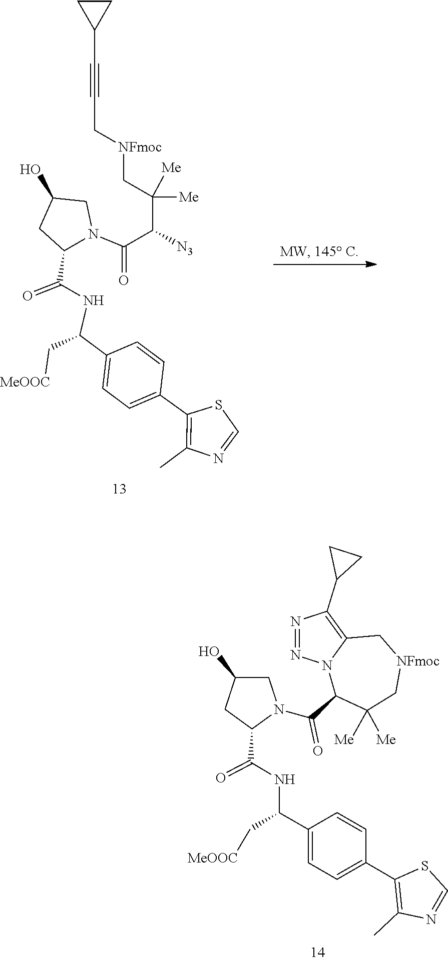

To a solution of 13 (1 equiv.) in 1,2-DCB (c₁₃=0.01 mol/L). Microwave the reaction at 145° C. for 16 h. The reaction was purified by silica column chromatography to afford 14 UPLC-MS: calculated [M+H]⁺: 844.4, found: 844.4.

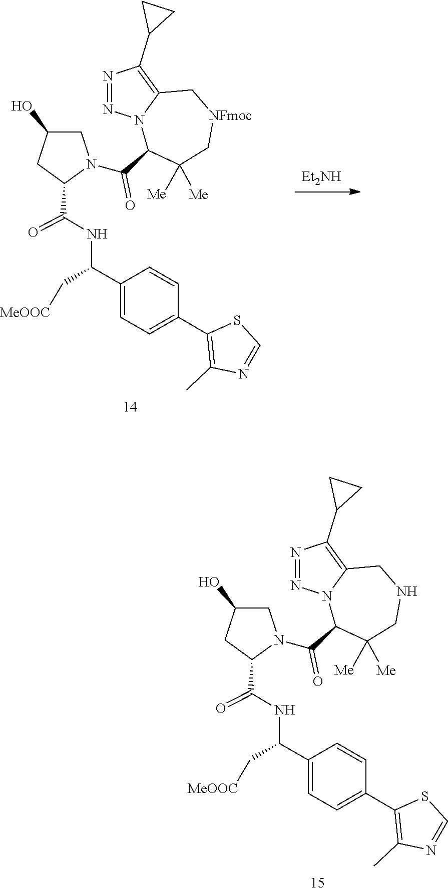

To a solution of 14 (1 equiv.) MeCN. Then diethylamine (5 equiv.) was added. The mixture was stirred for 3 hours. The mixture was detected by UPLC-MS and purified by HPLC to afford A10. UPLC-MS: calculated [M+H]⁺: 622.3, found: 622.5.

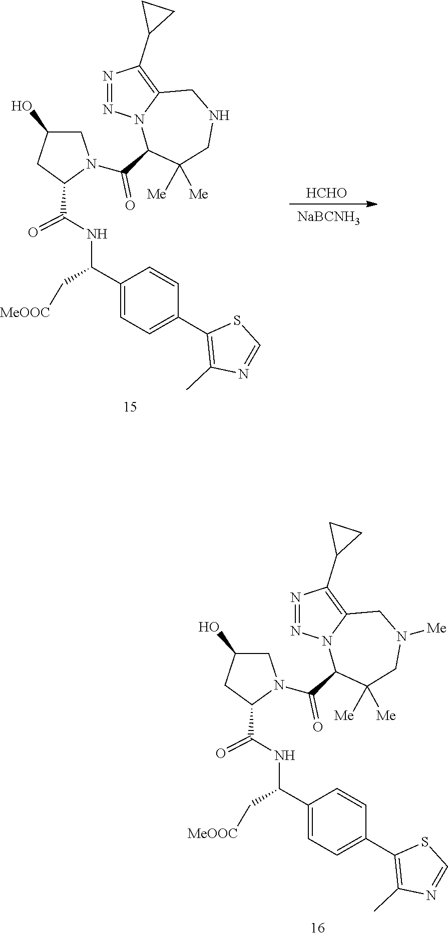 [SMILES: COC(=O)C[C@H](NC(=O)[C@@H]1C[C@@H](O)CN1C(=O)[C@H]1n2nnc(C3CC3)c2CNCC1(C)C)c1ccc(-c2scnc2C)cc1 ; "NaBCNH3" ; "HCHO" ; COC(=O)C[C@H](NC(=O)[C@@H]1C[C@@H](O)CN1C(=O)[C@H]1n2nnc(C3CC3)c2CN(C)CC1(C)C)c1ccc(-c2scnc2C)cc1]

To a solution of 15 (1 equiv.) in MeOH (c₁₅=0.1 mol/L), HCHO aq. (37% formaldehyde solution, 13.4 mol/L, 5 equiv) was added. The mixture was stirred for 30 min. Then NaBH₃CN (3 equiv.) was added in 5 portions. The mixture was detected by UPLC-MS and purified by HPLC to afford A19. UPLC-MS: calculated [M+H]⁺: 636.3, found: 636.4.

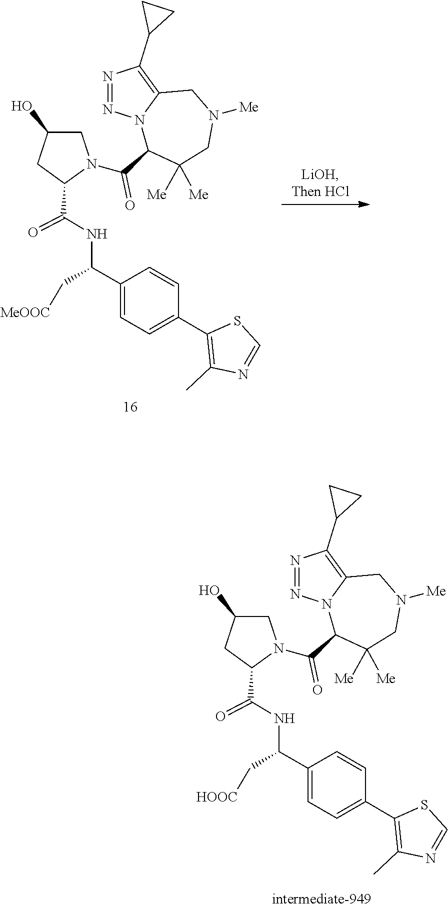 [SMILES: COC(=O)C[C@H](NC(=O)[C@@H]1C[C@@H](O)CN1C(=O)[C@H]1n2nnc(C3CC3)c2CN(C)CC1(C)C)c1ccc(-c2scnc2C)cc1 ; Cc1ncsc1-c1ccc([C@H](CC(=O)O)NC(=O)[C@@H]2C[C@@H](O)CN2C(=O)[C@H]2n3nnc(C4CC4)c3CN(C)CC2(C)C)cc1]

To a solution of 16 (1 equiv.) in MeOH/H₂O (c₁₅=0.1 mol/L, 3:1). LiOH (3 equiv) was added, the mixture was stirred for 3 h. Then the reaction was quenched by HCL (4 mol/L). The reaction was purified by HPLC to afford Intermediate-949.

### Part III: Compound B5, B6, B12, and B13.

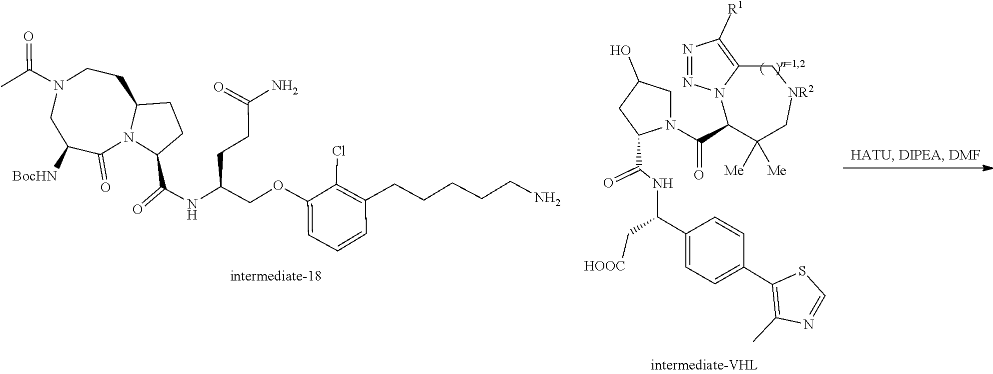

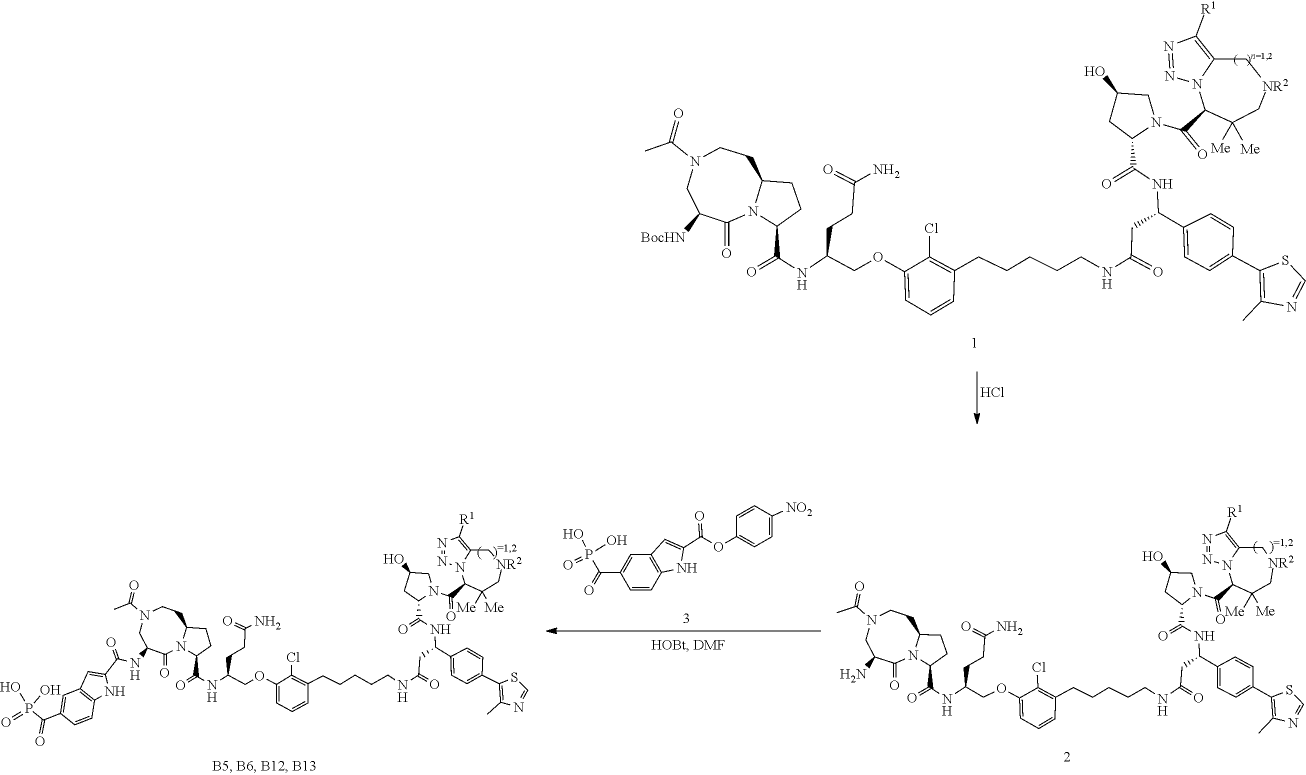

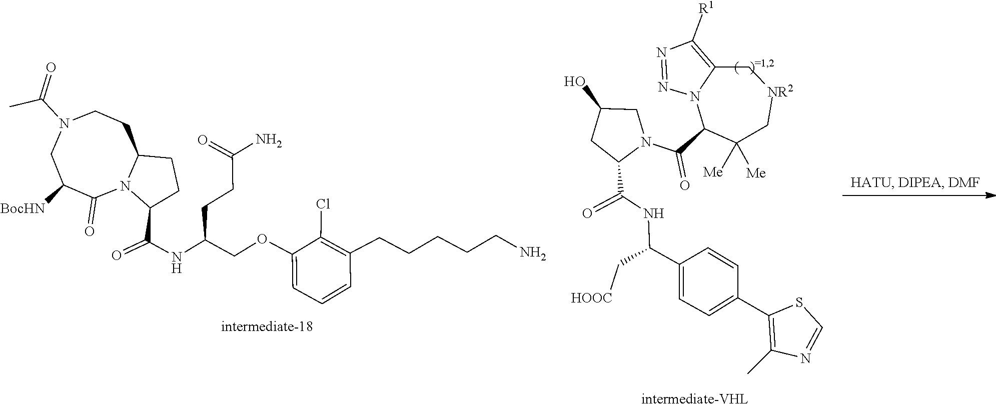

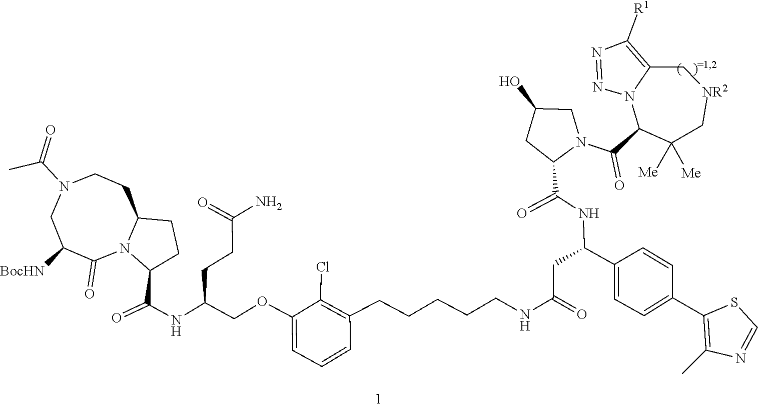

To a solution of Intermediate-18 (1 equiv.) and Intermediate-VHL (1 equiv.) in DMF. Then DIPEA (5 equiv.) and HATU (1 equiv.) were added. The mixture was stirred for 10 min. The mixture was detected by UPLC-MS and purified by HPLC to afford 1.

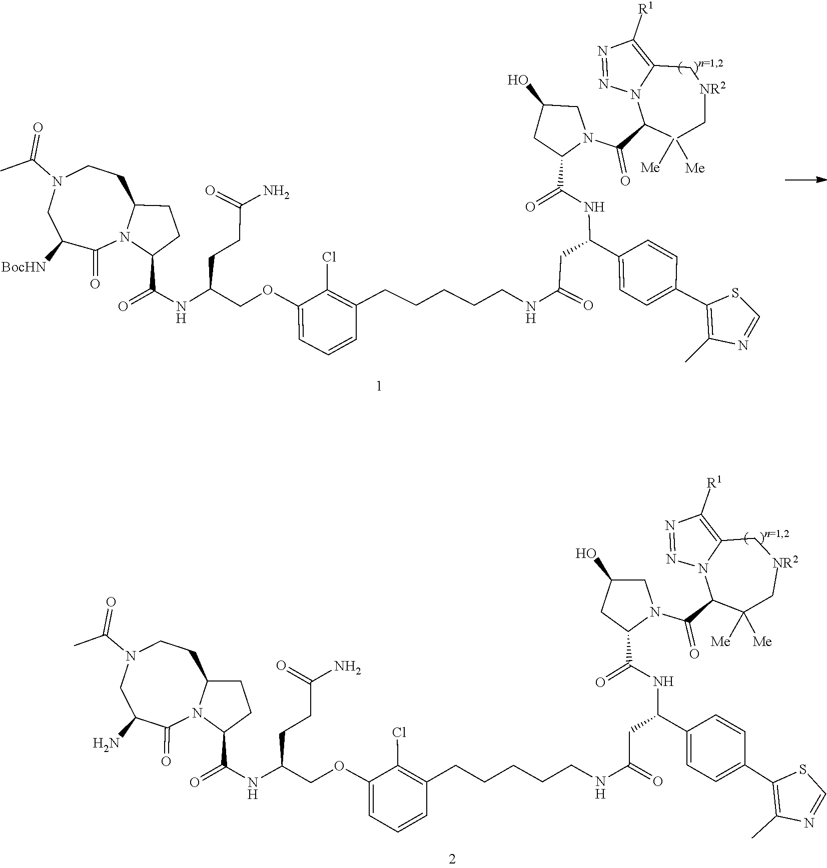

1 (1 equiv.) was dissolved in CH₂Cl₂, HCl (4M in dioxane, 5 equiv.) was added. The reaction was stirred for one hours. Solvent was removed in vacuum for next step without further purification.

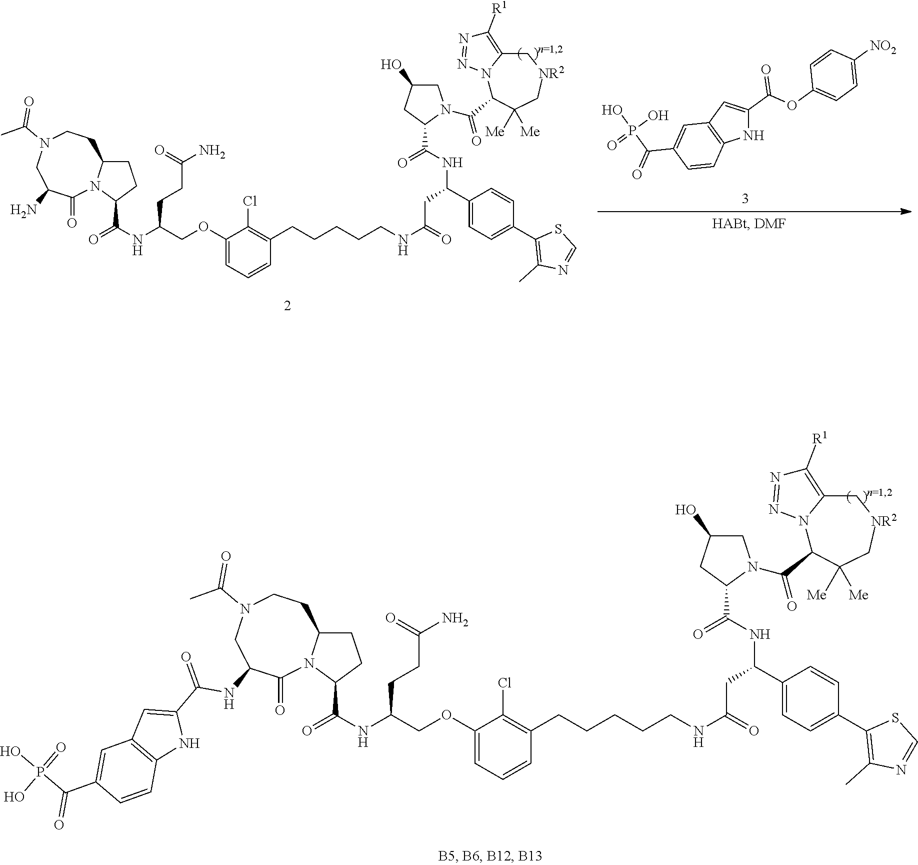

2 (1 equiv.) was dissolved in DMF. Then 3 (1 equiv.) and HOBt (1 equiv.) was added. The reaction was stirred for one hour. Then purified by HPLC to afford B5, B6, B12, and B13. UPLC-MS:
B5: calculated [M+2H]/2⁺: 731.3, found: 731.8.
B6: calculated [M+2H]/2⁺: 718.3, found: 718.8.
B12: calculated [M+2H]/2⁺: 717.3, found: 717.8.
B13: calculated [M+2H]/2⁺: 725.3, found: 725.7.

### Compound B7. ((2-(((5S,8S,10aR)-3-acetyl-8-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)benzo[b]thiophen-5-yl)difluoromethyl)phosphonic acid

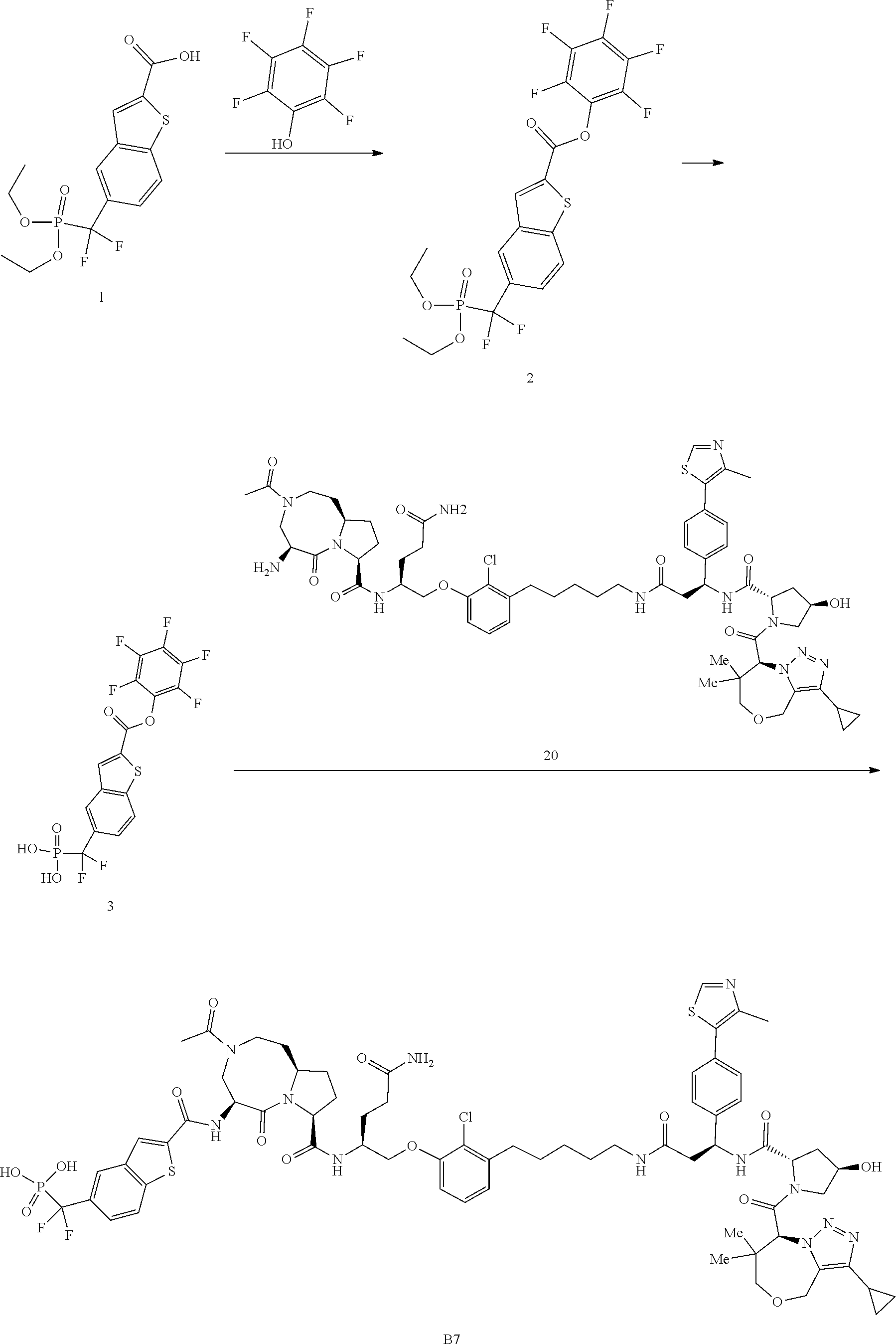

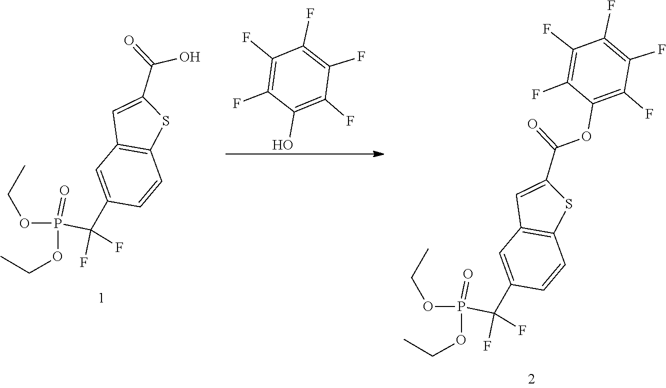 [SMILES: CCOP(=O)(OCC)C(F)(F)c1ccc2sc(C(=O)O)cc2c1 ; Oc1c(F)c(F)c(F)c(F)c1F ; CCOP(=O)(OCC)C(F)(F)c1ccc2sc(C(=O)Oc3c(F)c(F)c(F)c(F)c3F)cc2c1]

A solution of 1 (276 mg, 0.76 mmol, 1 equiv.), pentafluorophenol (155 mg, 0.84 mmol, 1.1 equiv.), DCC (204 mg, 0.99 mmol, 1.3 equiv.), and DMAP (9.3 mg, 0.64 mmol, 0.1 equiv.) in 3 mL of DMF was stirred at room temperature for 24 h. The mixture was directly purified by HPLC to yield compound 2 (265 mg, yield: 71%). UPLC-MS calculated [M+H]⁺: 531.0, found: 531.3.

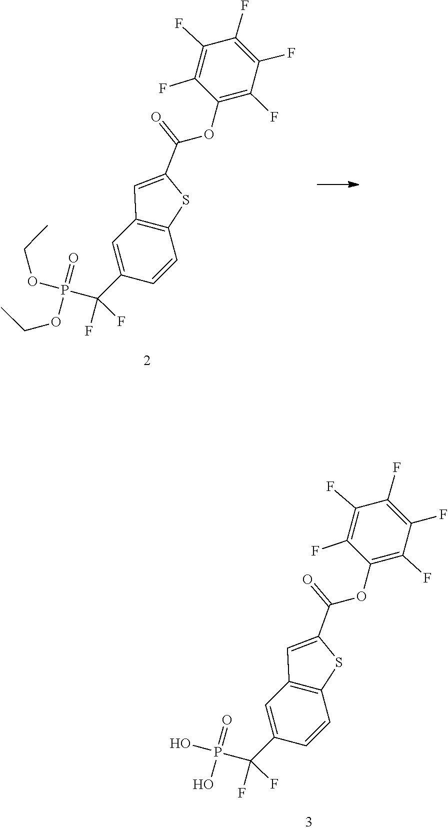 [SMILES: CCOP(=O)(OCC)C(F)(F)c1ccc2sc(C(=O)Oc3c(F)c(F)c(F)c(F)c3F)cc2c1 ; O=C(Oc1c(F)c(F)c(F)c(F)c1F)c1cc2cc(C(F)(F)P(=O)(O)O)ccc2s1]

To a round bottom flask was added 2 (265 mg, 0.5 mmol, 1.0 equiv) and CH₂Cl₂ (10 mL). The solution was cooled to 0° C. before adding CF₃CON(TMS)₂ (518 mg, 2.0 mmol, 4.0 equiv) and 1M of TMS-I in DCM (1.0 mL, 1.0 mmol, 2.0 equiv). The reaction mixture was allowed to stir at 0° C. until the starting material disappear and the solvent was removed under vacuum at 0° C. The residue was dissolved in a mixture solvent of CH₃CN and water, and purified by HPLC to yield 3 (192 mg, yield: 81%). UPLC-MS calculated [M+H]⁺: 475.0, found: 475.2.

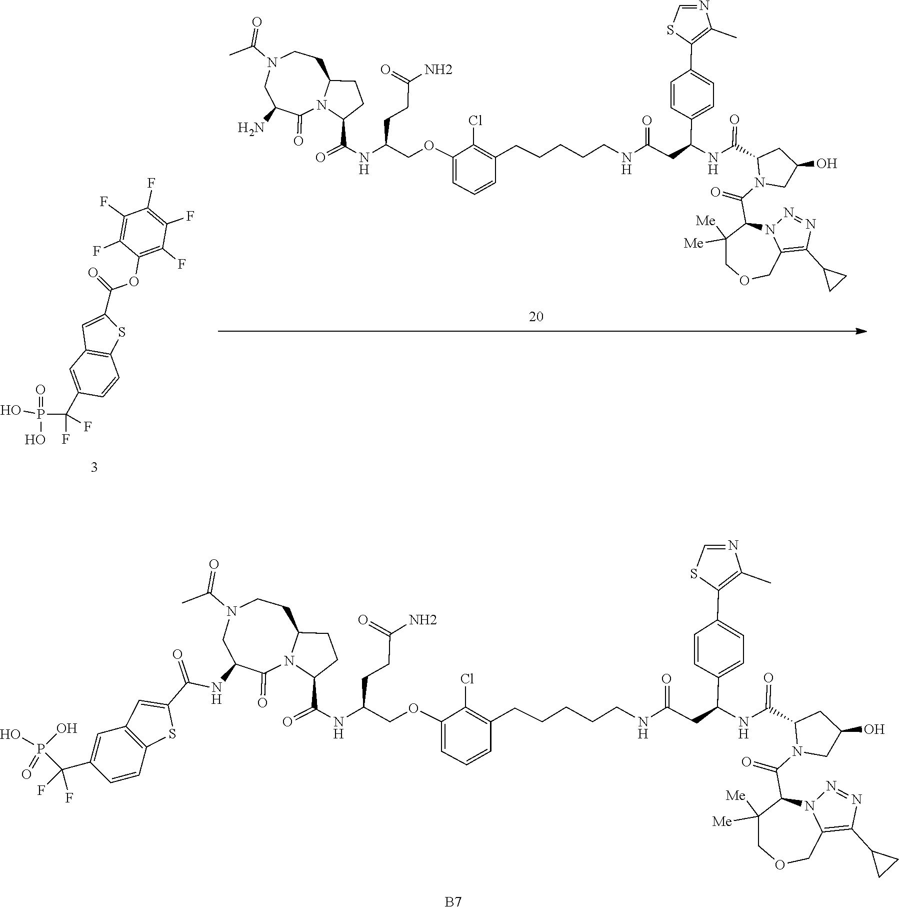 [SMILES: O=C(Oc1c(F)c(F)c(F)c(F)c1F)c1cc2cc(C(F)(F)P(=O)(O)O)ccc2s1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](N)C1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(F)(F)P(=O)(O)O)ccc3s2)C1 ; "B7"]

DIEA (0.042 mmol, 3 equiv.) was added to the mixture of 3 (0.017 mmol, 1.2 equiv.), compound 20 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B7. UPLC-MS calculated [M+2H]²⁺: 730.3, found: 730.4. Retention time: 3.5 min.

### Compound B8. (2-(((5S,8S,10aR)-3-acetyl-8-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(2,4-dimethylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

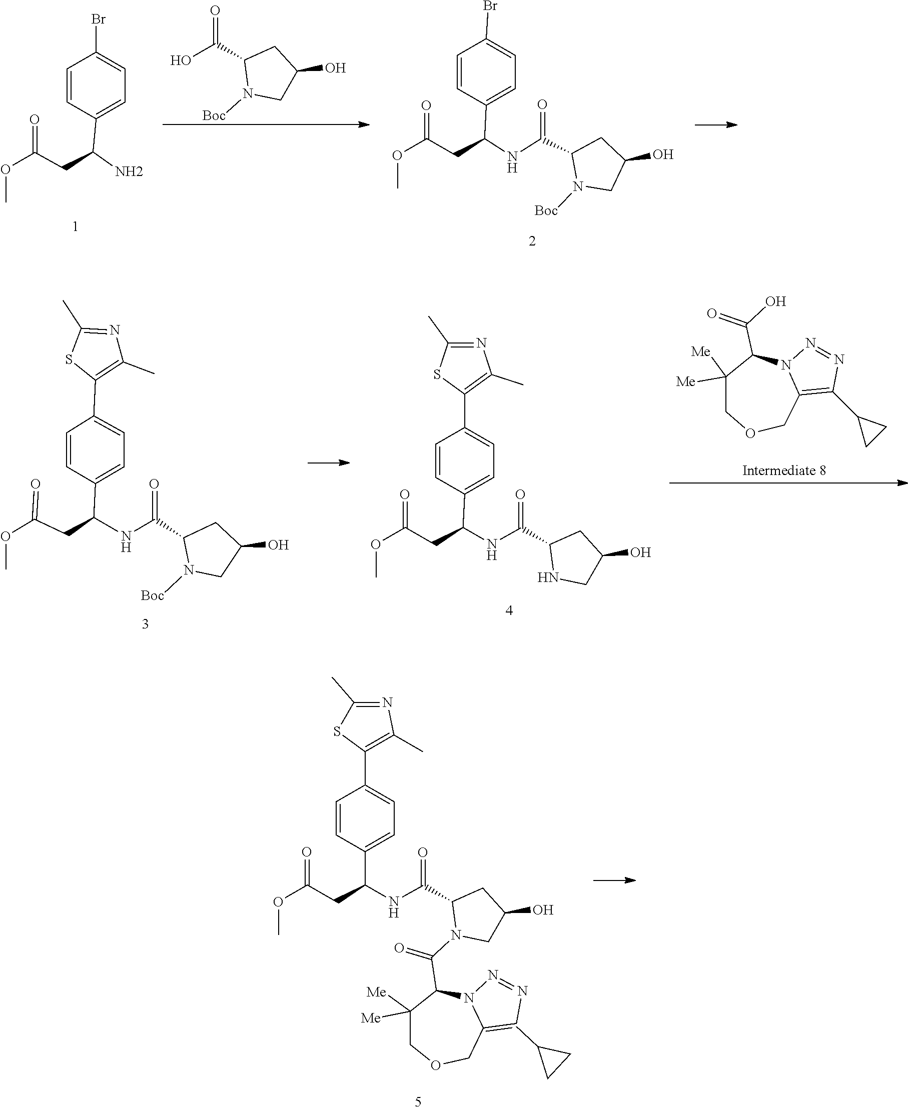

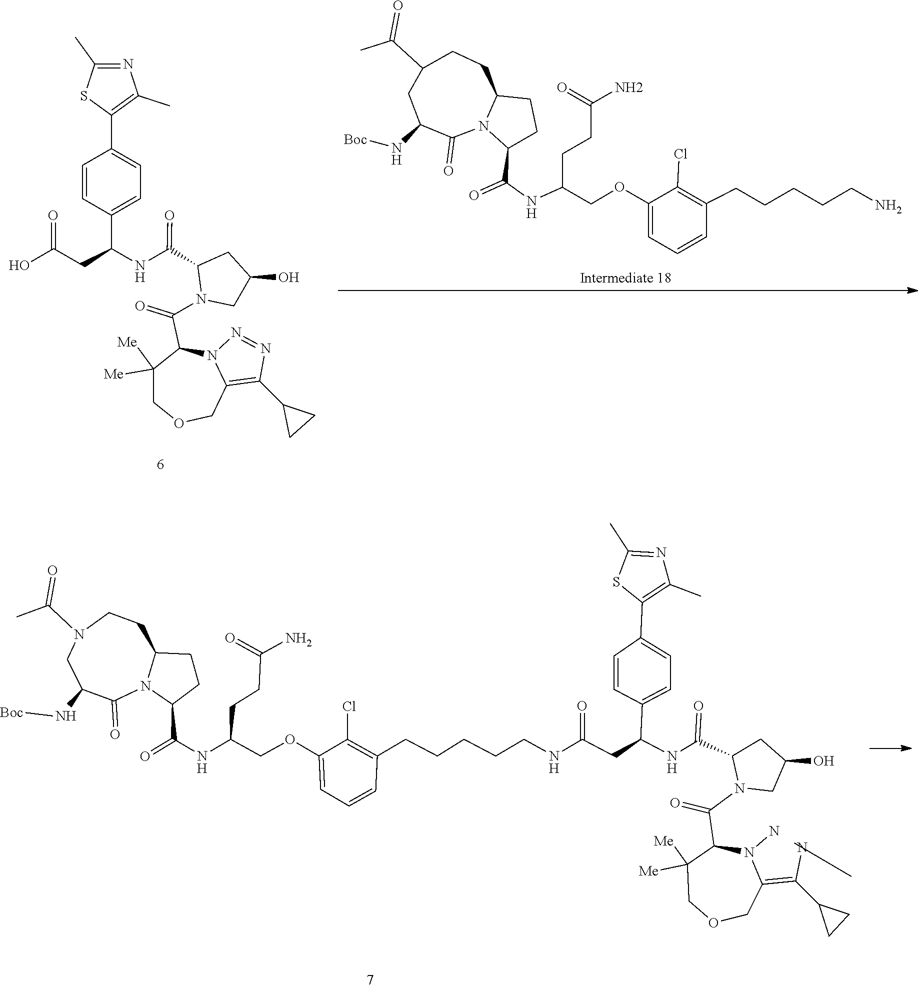

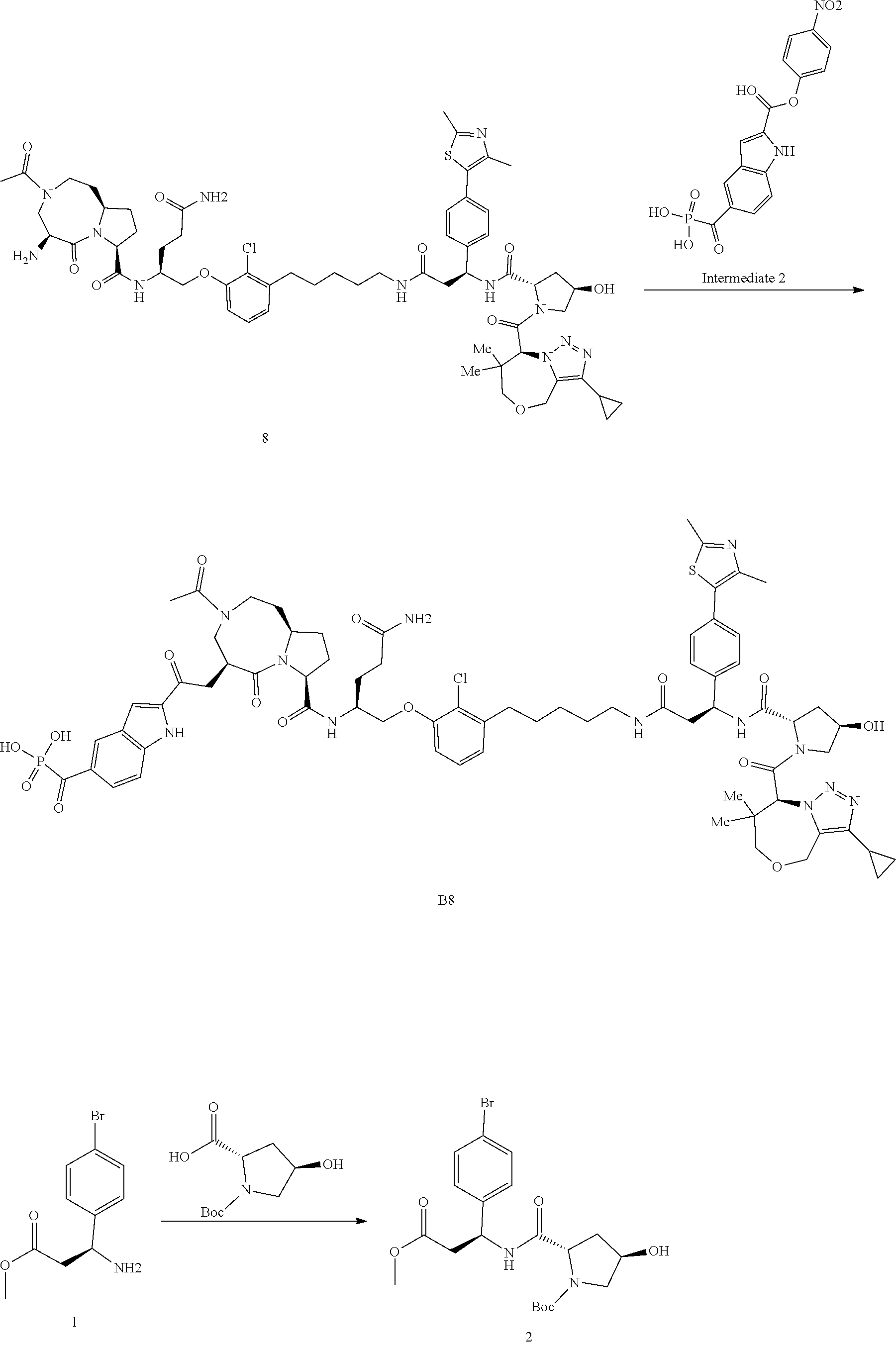

HATU (1.65 mmol, 1.1 equiv.) was added to a solution of 1 (1.5 mmol, 1 equiv.), (2S,4R)-1-(tert-butoxycarbonyl)-4-hydroxypyrrolidine-2-carboxylic acid (1.5 mmol, 1 equiv.) and DIEA (4.5 mmol, 3 equiv.) in DMF (15 mL) and the resulting mixture was stirred at rt for 1 h. This mixture was purified by reverse phase preparative HPLC to give 2. UPLC-MS calculated [M+H]+: 471.1, found: 471.3 Retention time: 3.8 min.

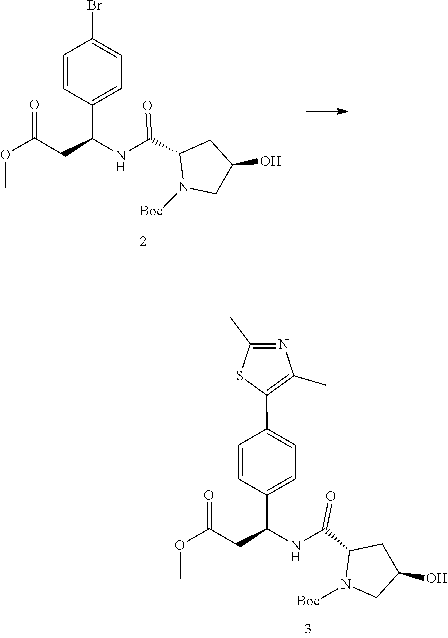 [SMILES: COC(=O)C[C@H](NC(=O)[C@@H]1C[C@@H](O)CN1C(=O)OC(C)(C)C)c1ccc(Br)cc1 ; COC(=O)C[C@H](NC(=O)[C@@H]1C[C@@H](O)CN1C(=O)OC(C)(C)C)c1ccc(-c2sc(C)nc2C)cc1]

Pd(OAc)₂ (0.1 mmol, 0.11 equiv.) was added to a mixture of compound 2 (1.0 mmol, 1 equiv.), 2,4-dimethylthiazole (2 mmol, 2 equiv.) and Potassium acetate (2 mmol, 2 equiv) in DMF (5 mL). The resulting mixture was purged and refilled with argon three times and stirred at 100° C. overnight under Argon. The reaction mixture was then cooled to room temperature and evaporated to remove most of the solvent. The residue was purified by HPLC to yield the 3 UPLC-MS calculated [M+H]+: 504.2, found: 504.4. Retention time: 3.2 min.

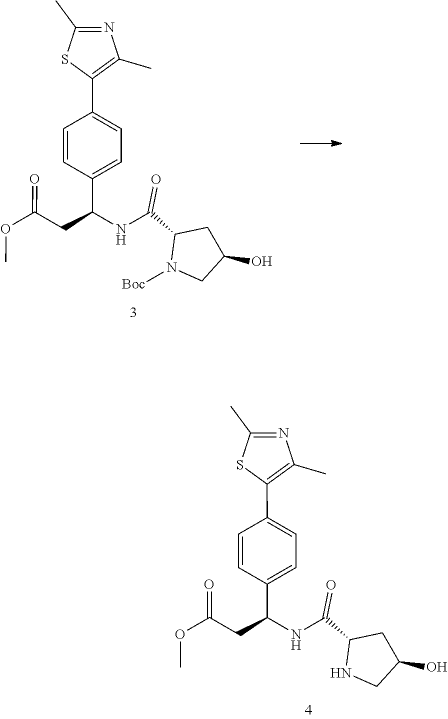 [SMILES: COC(=O)C[C@H](NC(=O)[C@@H]1C[C@@H](O)CN1C(=O)OC(C)(C)C)c1ccc(-c2sc(C)nc2C)cc1 ; COC(=O)C[C@H](NC(=O)[C@@H]1C[C@@H](O)CN1)c1ccc(-c2sc(C)nc2C)cc1]

Compound 3 (250 mg) was treated with 5 mL of 1M HCl (in Methanol) and 2 mL of 4M HCl (in dioxane) for 2 h. The reaction mixture was evaporated to give the 4 without further purification. UPLC-MS calculated [M+H]⁺: 404.2, found: 404.5. Retention time: 1.8 min.

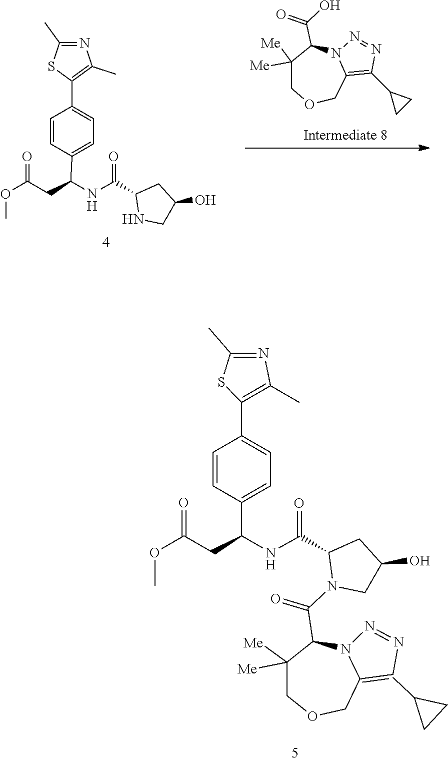

HATU (0.11 mmol, 1.1 equiv.) was added to a solution of Intermediate 8 (0.1 mmol, 1 equiv.), 4 (0.1 mmol, 1 equiv.) and DIEA (0.3 mmol, 3 equiv.) in DMF (1 mL) and the resulting mixture was stirred at rt for 1 h. This mixture was purified by reverse phase preparative HPLC to give 5. UPLC-MS calculated [M+H]⁺: 637.3, found: 637.6. Retention time: 3.6 min.

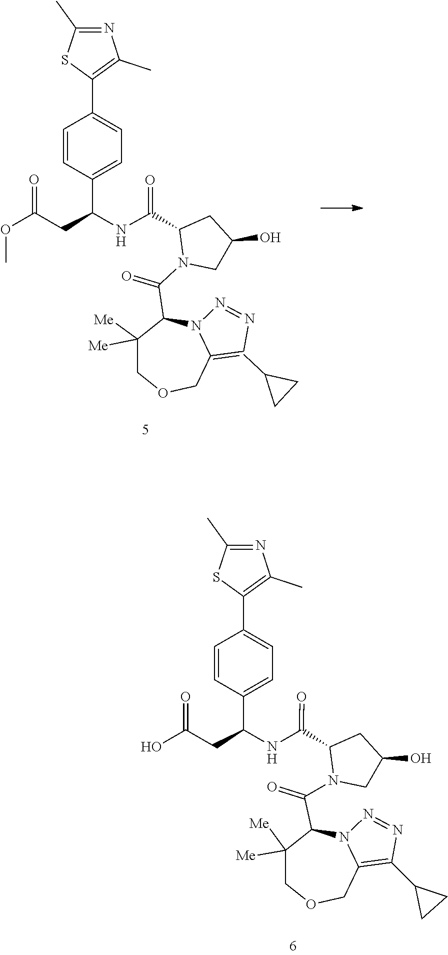

LiOH (0.2 mmol in 1 mL H2O) was added to a solution of 5 (0.1 mmoL) in Methanol (1 ml) and the resulting mixture was stirred for 2 h. Acidified with 1M HCl and then purification was performed by HPLC to give 6. UPLC-MS calculated [M+H]⁺: 623.3, found: 623.7.

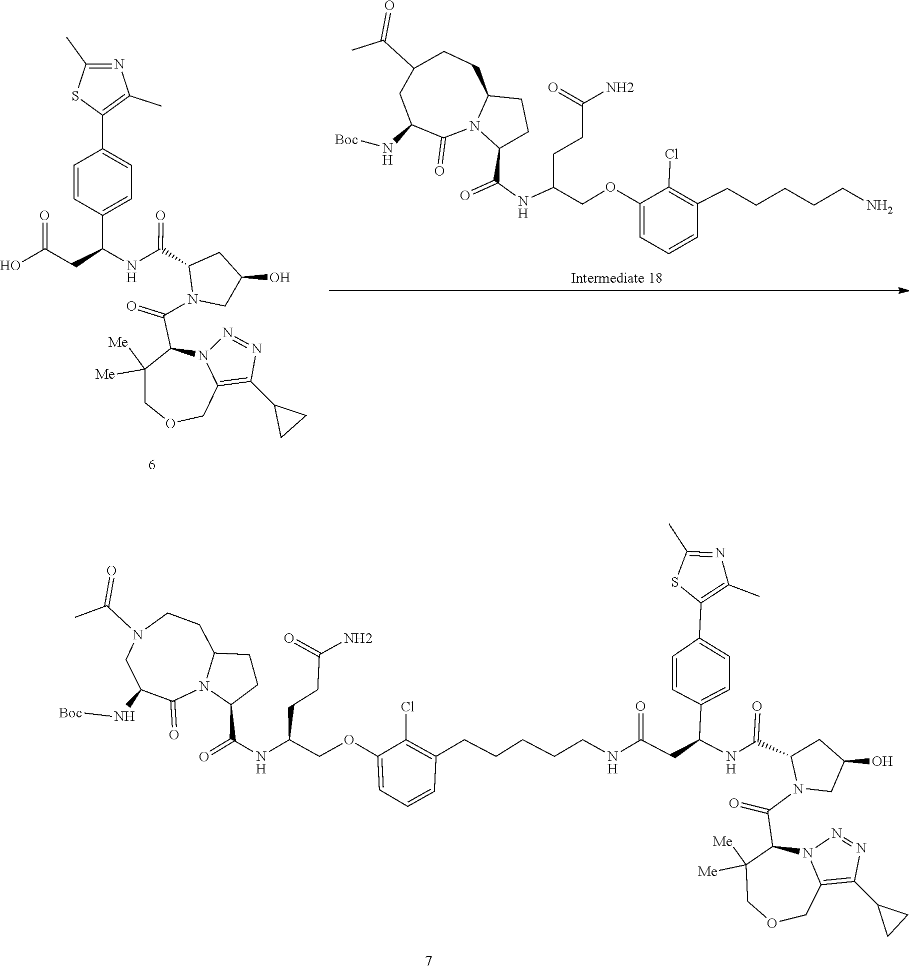

HATU (0.055 mmol, 1.1 equiv.) was added to a solution of 6 (0.05 mmol, 1 equiv.), intermediate 18 (0.05 mmol, 1 equiv.) and DIEA (0.15 mmol, 3 equiv.) in DMF (2 mL) and the resulting mixture was stirred at rt for 1 h. This mixture was purified by reverse phase preparative HPLC to yield 7. UPLC-MS calculated [M+2H]²⁺: 642.3, found: 642.6.

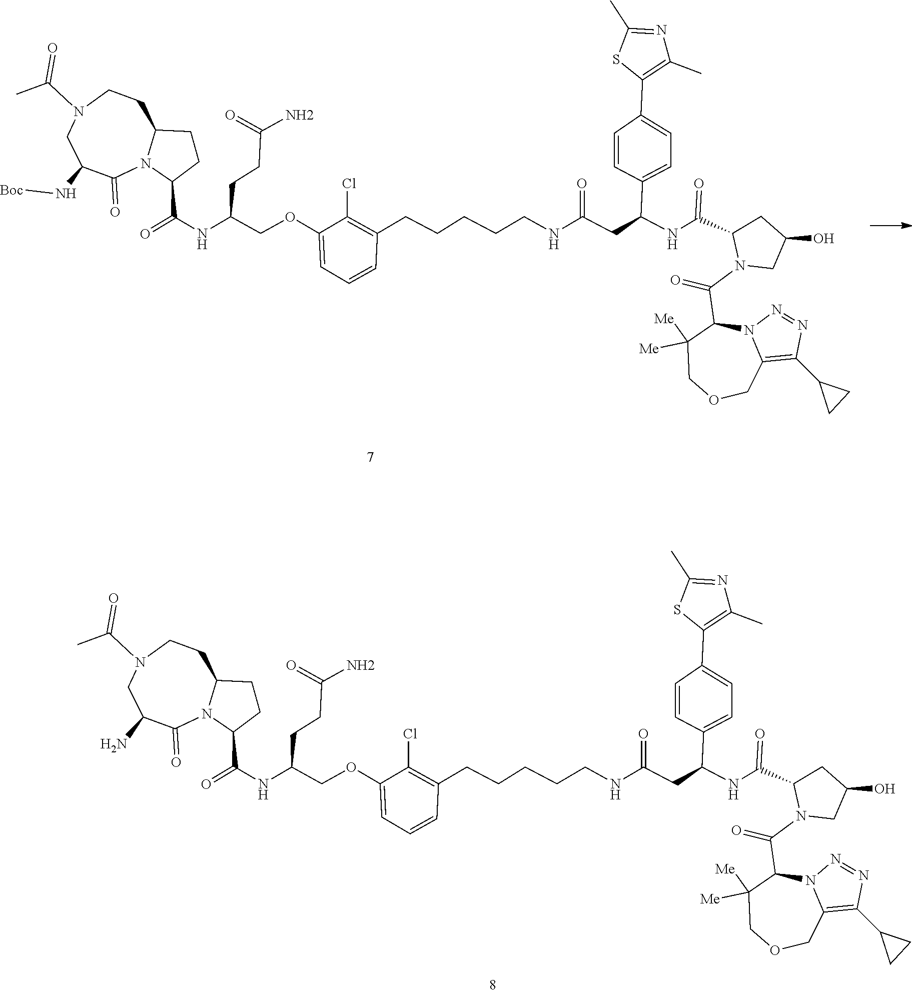 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](NC(=O)OC(C)(C)C)C1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](N)C1]

Compound 7 (50 mg) was treated with 1 mL of 1M HCl (in Methanol) and 1 mL of 4M HCl (in dioxane) for 2 h. The reaction mixture was evaporated to give the 8 without further purification. UPLC-MS calculated [M+H]⁺: 1183.5, found: 1183.8.

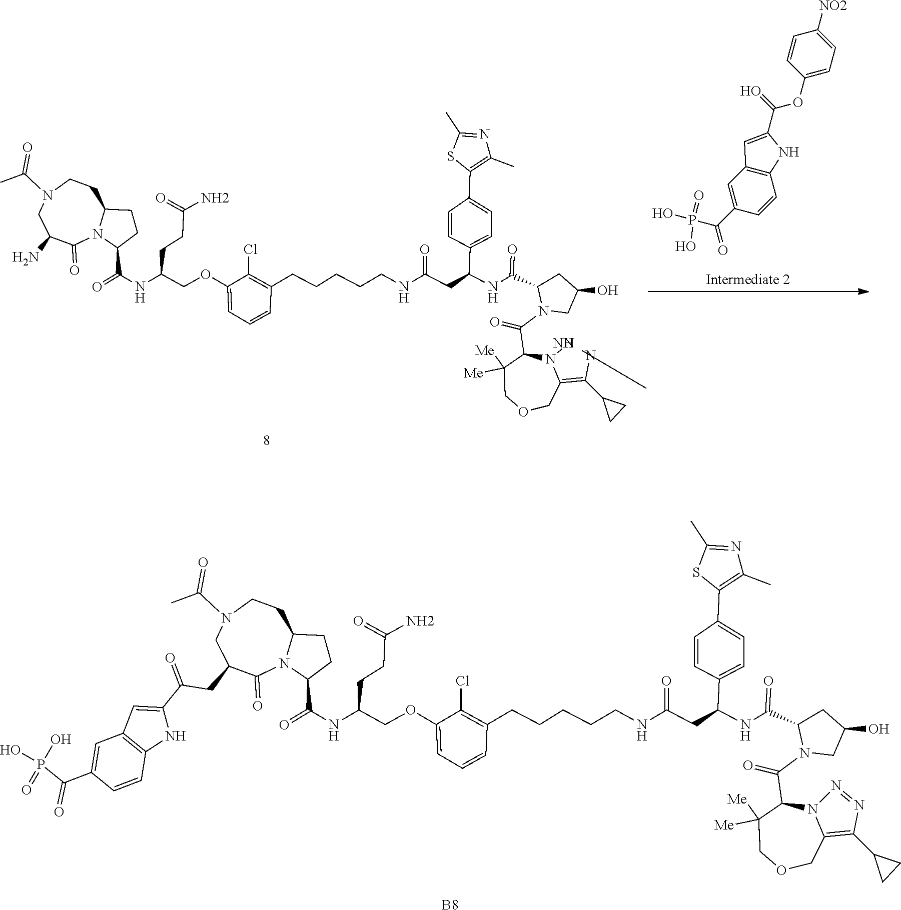

DIEA (0.06 mmol, 3 equiv.) was added to the mixture of Intermediate 2 (0.024 mmol, 1.2 equiv.), compound 8 (0.02 mmol, 1 equiv.) and HOBt (0.04 mmol, 2 equiv.) in DMF (1 mL). The resulted mixture was stirred at room temperature for 1 hour. Purification of this reaction mixture by HPLC gave the compound B8. UPLC-MS calculated [M+2H]²⁺: 717.8, found: 717.9. Retention time: 3.4 min.

### Compound B9. ((2-(((5S,8S,10aR)-3-acetyl-8-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(2,4-dimethylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)benzo[b]thiophen-5-yl)difluoromethyl)phosphonic acid

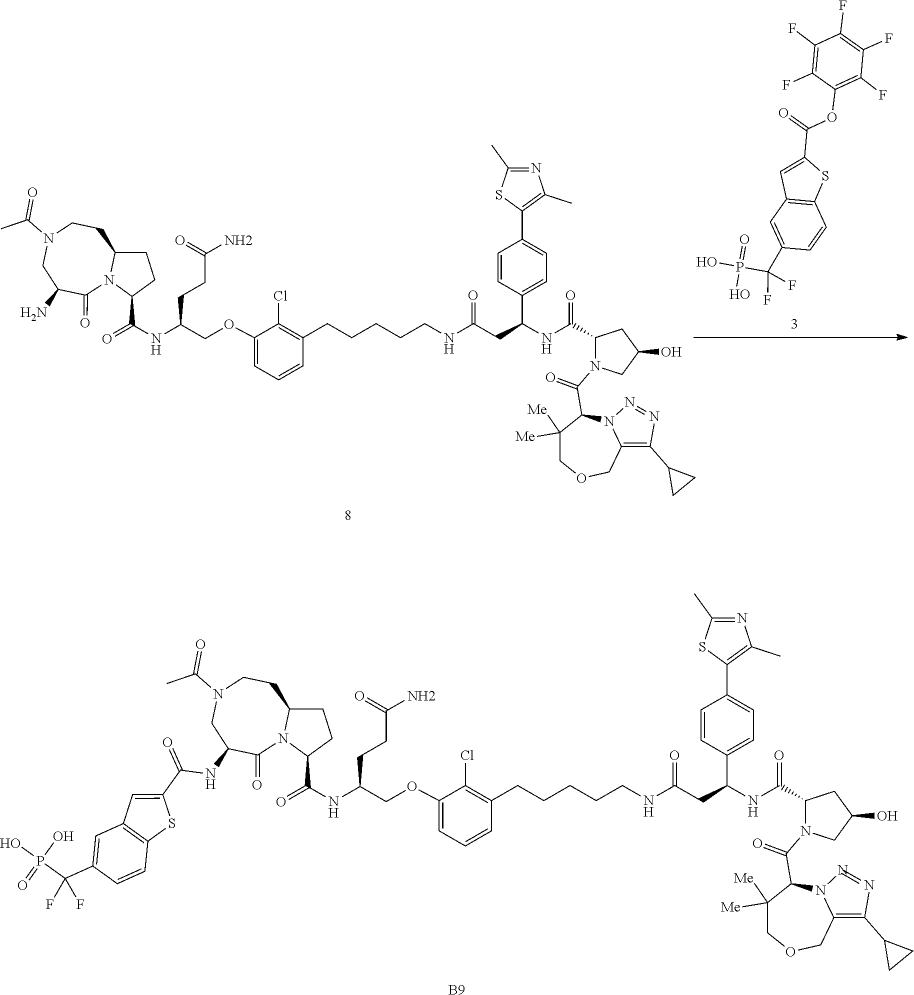

DIEA (0.042 mmol, 3 equiv.) was added to the mixture of 3 (0.017 mmol, 1.2 equiv.), compound 8 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B9. UPLC-MS calculated [M+2H]²⁺: 737.3, found: 737.8. Retention time: 3.6 min.

### Compound B11. (2-(((5S,8S,10aR)-3-acetyl-8-(((S)-5-amino-1-(2-chloro-3-(5-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-5,6,7,8-tetrahydro-4H-cyclohepta[d]isoxazole-3-carboxamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

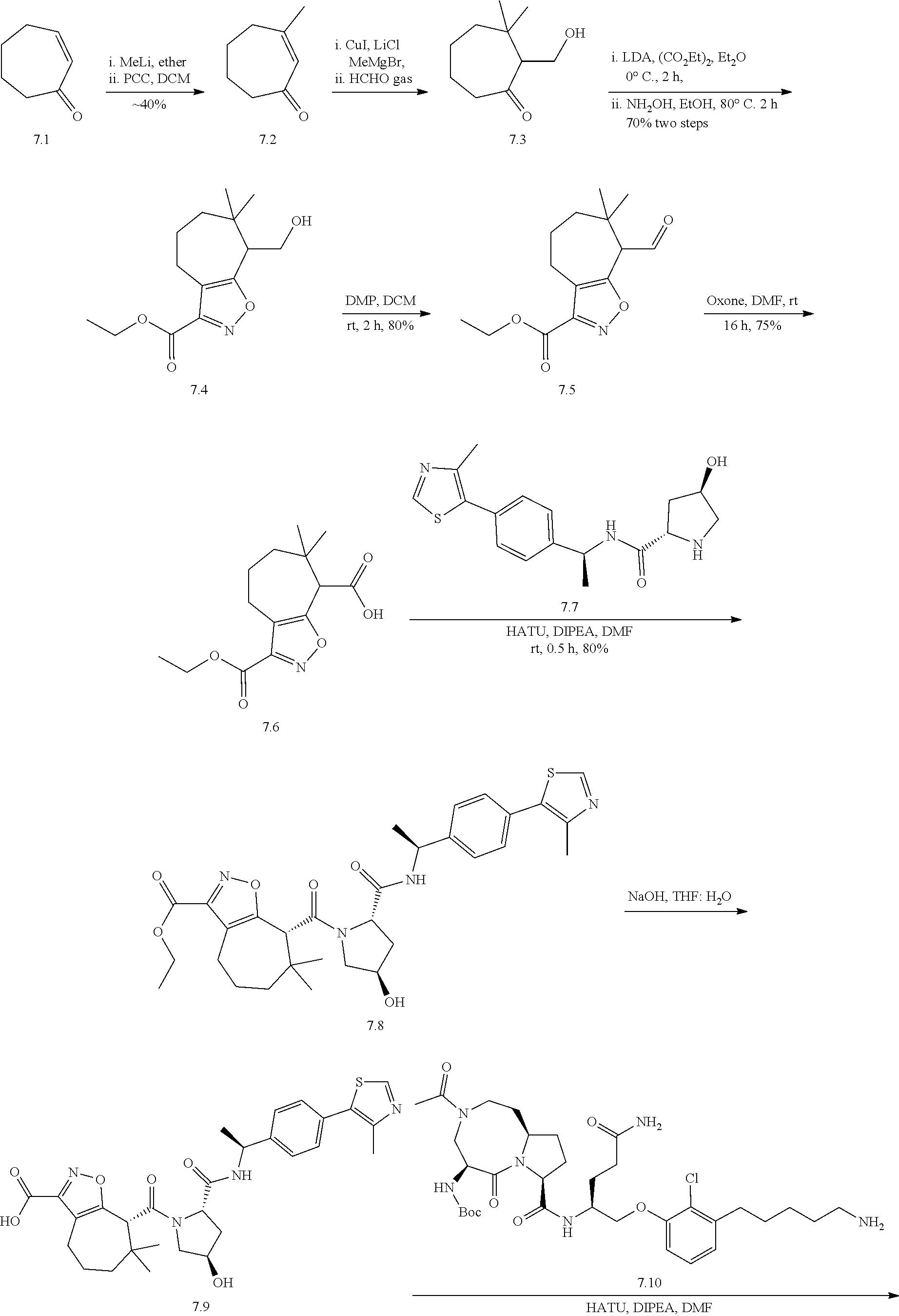

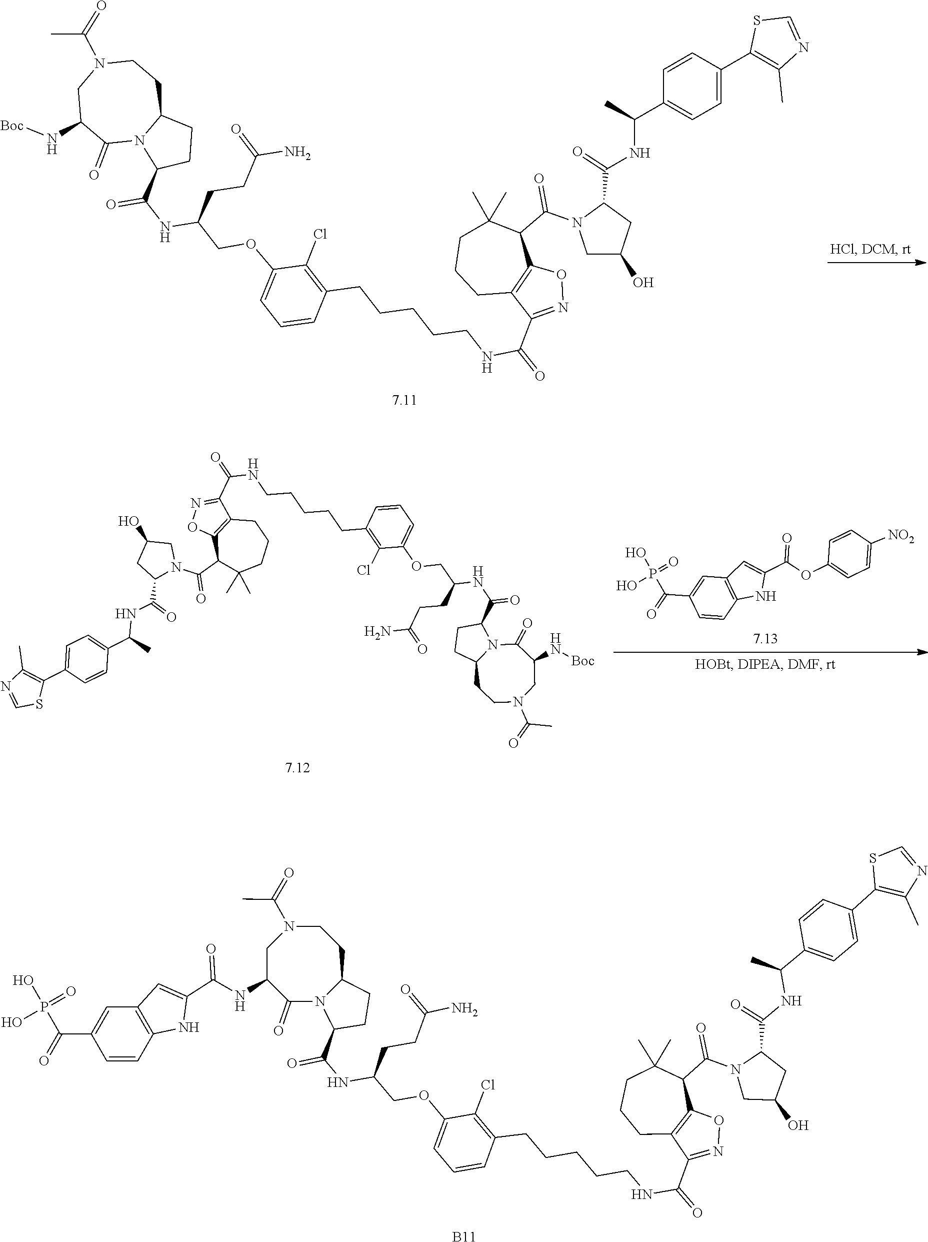

### Step 1

To a stirred solution of compound 7.1 in diethyl ether at −78° C., MeLi (1.1 eq) was added dropwise and the reaction was stirred at the same temperature for another 2 h. After complete conversion of the starting material, the reaction was quenched with saturated ammonium chloride and extracted with diethyl ether. The organic layer was washed with brine and dried over Na₂SO₄. The crude product was used for the next step.

To a stirred solution of the crude product in DCM at rt, PCC (1.5 eq) was added and the reaction was vigorously stirred for 2 h. The reaction was monitored by TLC chromatography. After complete conversion of the starting material, the reaction was diluted with ethyl ether and filtered through a pad of celite. The mixture was washed with ethyl ether. The filtrate was concentrated under reduced pressure and the product was purified with flash column chromatography. ¹H NMR (400 MHz, CDCl₃) δ 5.95 (d, J=1.9 Hz, 1H), 2.67-2.52 (m, 2H), 2.43 (dd, J=6.9, 4.6 Hz, 2H), 1.98 (dd, J=1.3, 0.7 Hz, 3H), 1.90-1.73 (m, 4H).

### Step 2

LiCl (0.1 eq) and CuI (0.05 eq) were added to a flame-dried two-way round bottom flask and purged with nitrogen three times. THF was added to the mixture and stirred vigorously until a homogeneous light-yellow solution. Compound 7.2 was added, and the reaction was cooled to 0° C. Methyl magnesium bromide (1.2 eq) was added to the reaction dropwise over 20 mins and the reaction was stirred at the same temperature for 2 h and monitored by TLC chromatography. After the complete conversion of starting material, the reaction was cooled to −10° C., and formaldehyde gas (generated by heating paraformaldehyde at 140° C.) was passed through the solution using a cannula for 4 h. The reaction was monitored by TLC chromatography. Upon completion of the reaction, the mixture was quenched with a saturated solution of ammonium chloride and extracted with ethyl acetate. The product was purified by flash column chromatography. ¹H NMR (400 MHz, CDCl₃) δ 4.02 (dd, J=11.2, 9.0 Hz, 1H), 3.71 (dd, J=11.2, 3.6 Hz, 1H), 2.96 (dd, J=9.0, 3.6 Hz, 1H), 2.67 (s, 2H), 2.59-2.34 (m, 2H), 1.89-1.73 (m, 3H), 1.66-1.40 (m, 2H), 1.05 (s, 3H), 0.83 (s, 3H).

### Step 3

A solution of compound 7.3 (1 eq), diethyl oxalate (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

A solution of crude product and hydroxyl amine hydrochloride (2 eq) in ethanol was heated to reflux for 2 h. After complete conversion of starting material, ethanol was evaporated under reduced pressure and the product was purified with flash column chromatography. ¹H NMR (400 MHz, CDCl₃) δ 4.41 (q, J=7.1 Hz, 2H), 3.81 (hept, J=6.7 Hz, 1H), 3.43 (hept, J=6.8 Hz, 1H), 3.14-3.03 (m, 1H), 2.98 (ddd, J=7.1, 5.9, 1.1 Hz, 1H), 2.60-2.44 (m, 1H), 2.06 (s, 2H), 1.88 (ddd, J=14.4, 11.1, 3.3 Hz, 1H), 1.71 (ddt, J=7.1, 6.0, 3.6 Hz, 1H), 1.61-1.53 (m, 1H), 1.40 (t, J=7.2 Hz, 3H), 1.05 (s, 3H), 0.93 (s, 3H). UPLC-MS: calculated [M+H]⁺: 268.15, found: 268.17.

### Step 4

To a stirred solution of compound 7.4 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography. ¹H NMR (400 MHz, CDCl₃) δ 9.85 (d, J=2.4 Hz, 1H), 4.41 (q, J=7.1 Hz, 2H), 3.76 (dd, J=2.4, 1.4 Hz, 1H), 3.16 (dddd, J=16.0, 5.2, 3.1, 1.2 Hz, 1H), 2.60-2.47 (m, 1H), 1.89 (ddd, J=14.0, 11.5, 2.7 Hz, 1H), 1.83-1.63 (m, 3H), 1.40 (t, J=7.1 Hz, 3H), 1.27 (s, 3H), 0.98 (s, 3H). UPLC-MS: calculated [M+H]⁺: 266.13, found: 266.07.

### Step 5

To a stirred solution of compound 7.5 (1 eq) in DMF at rt, Oxone monohydrate (3 eq) was added. The reaction was stirred at rt for 16 h followed by dilution with water. The mixture was extracted with ethyl acetate and the product was purified using flash column chromatography. UPLC-MS: calculated [M+H]⁺: 282.13, found: 282.04.

### Step 6

To a stirred solution of compound 7.6 (1 eq), compound 7.7 (1 eq), and HATU (1 eq) in DMF at rt, DIPEA (1.5 eq) was added dropwise and monitored by UPLC. After completion of the reaction, the product was purified by reverse phase column chromatography. UPLC-MS: calculated [M+H]⁺: 594.25, found: 595.10.

### Step 7

To a stirred solution of compound 7.8 in THF:water (1:1) at rt, NaOH was added and the reaction was stirred for 2 h. After complete conversion of the starting material, it was acidified with 2 N HCl up to pH 3 and extracted with ethyl acetate. The product was purified with flash column chromatography. UPLC-MS: calculated [M+H]⁺: 567.22, found: 567.12.

### Step 8

To a stirred solution of compound 7.9 (1 eq), compound 7.10 (1 eq), and HATU (1 eq) in DMF at rt, DIPEA (1.5 eq) was added dropwise and monitored by UPLC. After completion of the reaction, the product was purified by preparative HPLC (ACN: H₂O with 0.1% TFA). UPLC-MS: calculated [M+2H]²⁺/2: 614.28, found: 614.15.

### Step 9

To a stirred solution of 7.11 in DCM, 4 N HCl in dioxane was added and stirred for 1 h at room temperature and monitored by UPLC. After completion of the reaction solvent was evaporated to dryness and the crude product was used for the next step. UPLC-MS: calculated [M+H]⁺: 1127.51, found: 1127.15.

### Step 10

To a stirred solution of compound 7.12 (1 eq), compound 7.13 (1 eq), and HOBt (1 eq) in DMF at rt, DIPEA (1.5 eq) was added dropwise and monitored by UPLC. After completion of the reaction, the product was purified by preparative HPLC (ACN: H₂O with 0.1% TFA) to provide B11. UPLC-MS: calculated [M+2H]²⁺/2: 689.5, found: 689.9.

### Compound B14. ((2-(((3S,6S,10aS)-3-(((S)-5-amino-1-(2-chloro-3-(5-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-5-oxodecahydropyrrolo[1,2-a]azocin-6-yl)carbamoyl)benzo[b]thiophen-5-yl)difluoromethyl)phosphonic acid

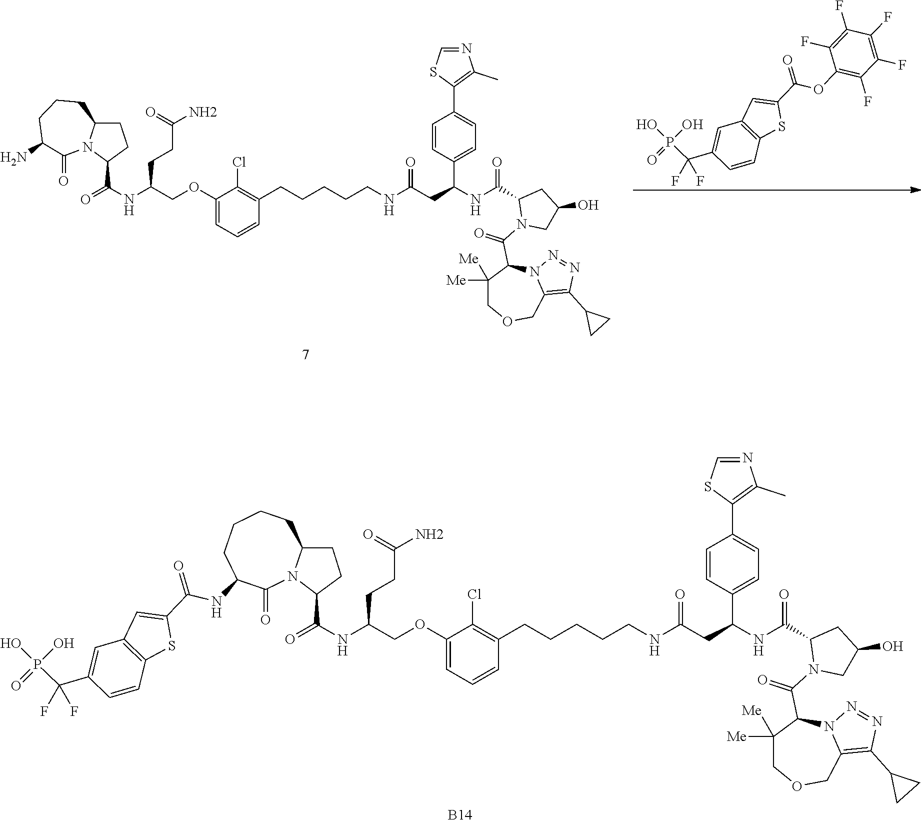 [SMILES: Cc1ncsc1-c1ccc([C@H](CC(=O)NCCCCCc2cccc(OC[C@H](CCC(N)=O)NC(=O)[C@@H]3CC[C@@H]4CCC[C@H](N)C(=O)N43)c2Cl)NC(=O)[C@@H]2C[C@@H](O)CN2C(=O)[C@H]2n3nnc(C4CC4)c3COCC2(C)C)cc1 ; O=C(Oc1c(F)c(F)c(F)c(F)c1F)c1cc2cc(C(F)(F)P(=O)(O)O)ccc2s1 ; Cc1ncsc1-c1ccc([C@H](CC(=O)NCCCCCc2cccc(OC[C@H](CCC(N)=O)NC(=O)[C@@H]3CC[C@@H]4CCCC[C@H](NC(=O)c5cc6cc(C(F)(F)P(=O)(O)O)ccc6s5)C(=O)N43)c2Cl)NC(=O)[C@@H]2C[C@@H](O)CN2C(=O)[C@H]2n3nnc(C4CC4)c3COCC2(C)C)cc1 ; "B14"]

DIEA (0.042 mmol, 3 equiv.) was added to the mixture of (difluoro (2-((perfluorophenoxy) carbonyl)benzo[b]thiophen-5-yl)methyl)phosphonic acid (0.017 mmol, 1.2 equiv.), compound 7 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B14. UPLC-MS calculated [M+2H]²⁺: 708.8, found: 708.9. Retention time: 4.0 min.

### Compound B15. (2-(((5S,8S,10aR)-3-acetyl-8-(((S)-5-amino-1-(2-chloro-3-(5-((S)-3-((2S,4R)-1-((S)-3-cyclopropyl-7,7-dimethyl-7,8-dihydro-4H,6H1-[1,2,3]triazolo[5,1-c][1,4]oxazepine-8-carbonyl)-4-hydroxypyrrolidine-2-carboxamido)-3-(4-(2,4-dimethyloxazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

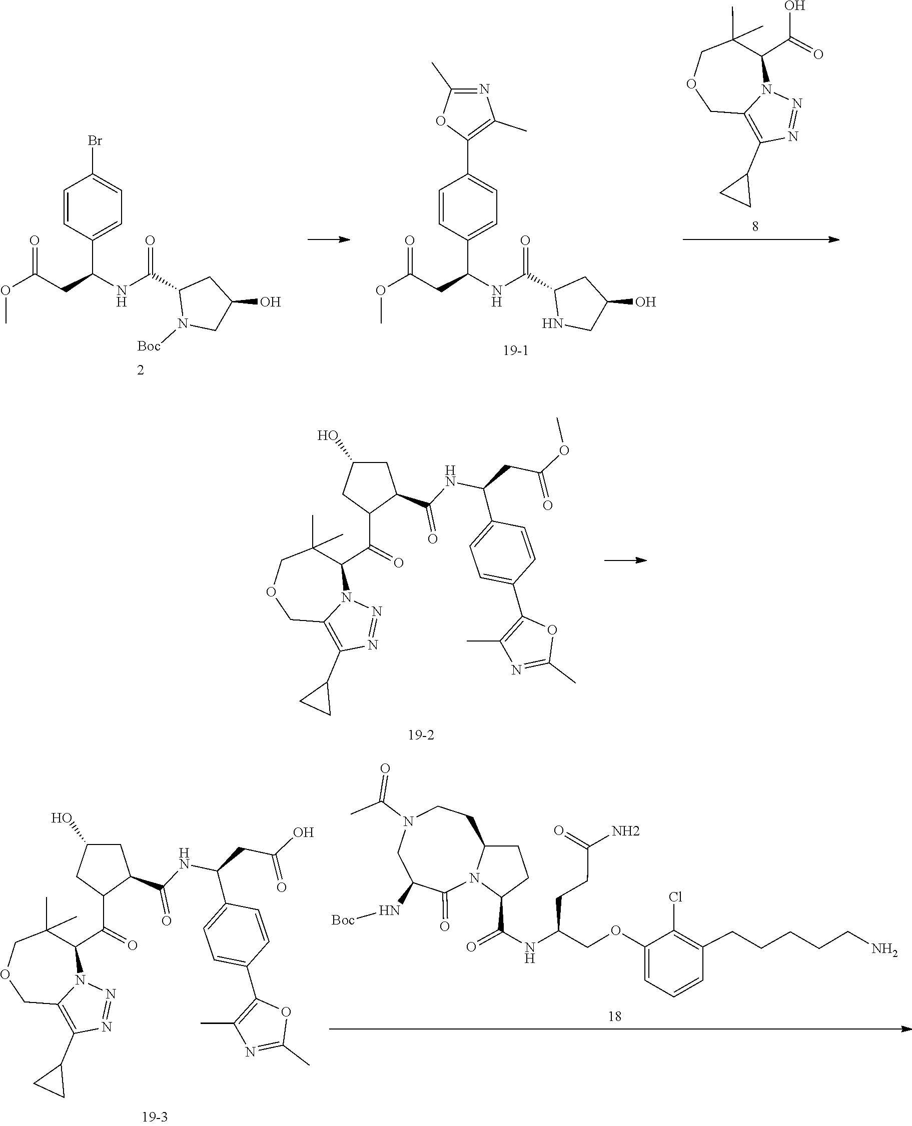 [SMILES: COC(=O)C[C@H](NC(=O)[C@@H]1C[C@@H](O)CN1C(=O)OC(C)(C)C)c1ccc(Br)cc1 ; COC(=O)C[C@H](NC(=O)[C@@H]1C[C@@H](O)CN1)c1ccc(-c2oc(C)nc2C)cc1 ; CC1(C)COCc2c(C3CC3)nnn2[C@@H]1C(=O)O ; COC(=O)C[C@H](NC(=O)[C@@H]1C[C@@H](O)CC1C(=O)[C@H]1n2nnc(C3CC3)c2COCC1(C)C)c1ccc(-c2oc(C)nc2C)cc1 ; Cc1nc(C)c(-c2ccc([C@H](CC(=O)O)NC(=O)[C@@H]3C[C@@H](O)CC3C(=O)[C@H]3n4nnc(C5CC5)c4COCC3(C)C)cc2)o1 ; CC(=O)N1CC[C@H]2CC[C@@H](C(=O)N[C@@H](CCC(N)=O)COc3cccc(CCCCCN)c3Cl)N2C(=O)[C@@H](NC(=O)OC(C)(C)C)C1]

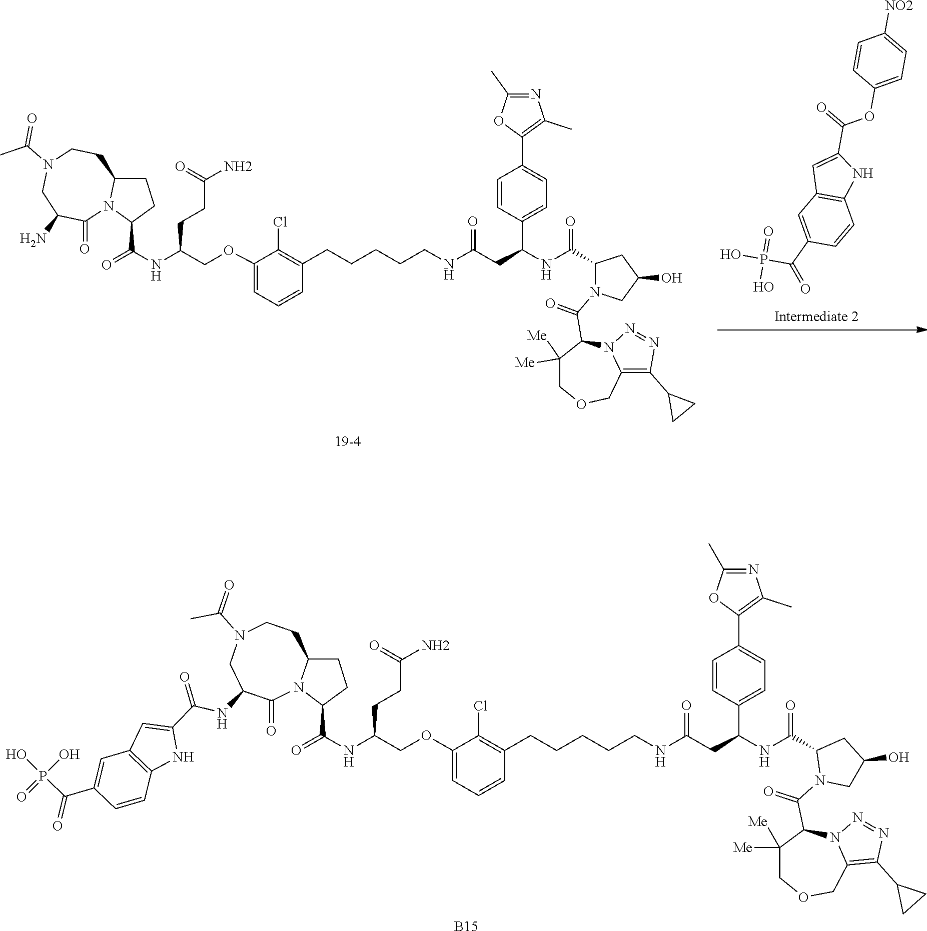 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5oc(C)nc5C)cc4)c3Cl)N2C(=O)[C@@H](N)C1 ; O=C(Oc1ccc([N+](=O)[O-])cc1)c1cc2cc(C(=O)P(=O)(O)O)ccc2[nH]1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5oc(C)nc5C)cc4)c3Cl)N2C(=O)[C@@H](NC(=O)c2cc3cc(C(=O)P(=O)(O)O)ccc3[nH]2)C1 ; "B15"]

19-1: Pd(OAc)₂ (0.1 mmol, 0.11 equiv.) was added to a mixture of compound 2 (1.0 mmol, 1 equiv.), 2,4-dimethyloxazole (2 mmol, 2 equiv.) and Potassium acetate (2 mmol, 2 equiv) in DMF (5 mL). The resulting mixture was purged and refilled with argon three times and stirred at 100° C. overnight under Argon. The reaction mixture was then cooled to room temperature and evaporated to remove most of the solvent. The residue was purified by HPLC and the obtained product was treated with 5 mL of 1M HCl (in Methanol) and 2 mL of 4M HCl (in dioxane) for 2 h. The reaction mixture was evaporated to give the 19-1 without further purification. UPLC-MS calculated [M+H]⁺: 388.2, found: 388.5. Retention time: 1.8 min.

19-2: HATU (0.022 mmol, 1.1 equiv.) was added to a solution of 19-1 (0.02 mmol, 1 equiv.), 8 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield 19-2. UPLC-MS calculated [M+H]²⁺: 621.3, found: 621.5. Retention time: 3.7 min.

19-3: LiOH (0.2 mmol in 1 mL H2O) was added to a solution of 19-2 (0.1 mmoL) in Methanol (1 ml) and the resulting mixture was stirred for 2 h. Acidified with 1M HCl and then purification was performed by HPLC to give 19-3. UPLC-MS calculated [M+H]⁺: 607.3, found: 607.5. Retention time: 3.1 min.

19-4: HATU (0.055 mmol, 1.1 equiv.) was added to a solution of 19-3 (0.05 mmol, 1 equiv.), intermediate 18 (0.05 mmol, 1 equiv.) and DIEA (0.15 mmol, 3 equiv.) in DMF (2 mL) and the resulting mixture was stirred at rt for 1 h. This mixture was purified by reverse phase preparative HPLC and the obtained product was treated with 5 mL of 1M HCl (in Methanol) and 2 mL of 4M HCl (in dioxane) for 2 h. The reaction mixture was evaporated to give the 19-4 without further purification. UPLC-MS calculated [M+H]⁺: 1167.6, found: 1167.9.

B15: DIEA (0.042 mmol, 3 equiv.) was added to the mixture of Intermediate 2 (0.017 mmol, 1.2 equiv.), compound 19-14 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B15. UPLC-MS calculated [M+2H]²⁺: 709.8, found: 710.0. Retention time: 3.4 min.

### Compound B16. (2-(((5S,8S,10aR)-8-(((S)-5-amino-1-((4-(ethylsulfonyl)benzyl)amino)-1,5-dioxopentan-2-yl)carbamoyl)-3-(6-((S)-3-((2R,4S)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)hexanoyl)-6-oxodecahydropyrrolo[1,2-a][1,5]diazocin-5-yl)carbamoyl)-1H-indole-5-carbonyl)phosphonic acid

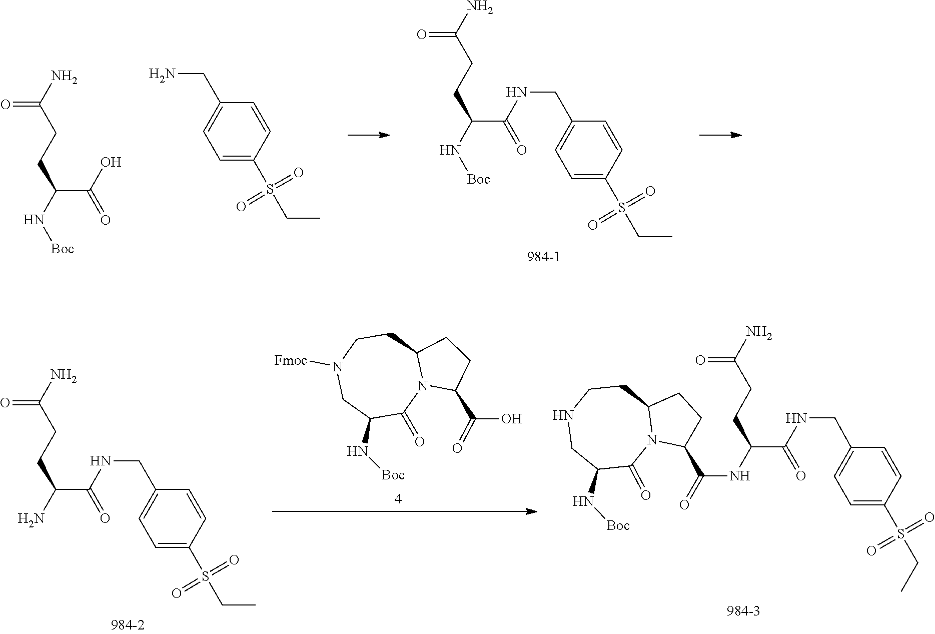

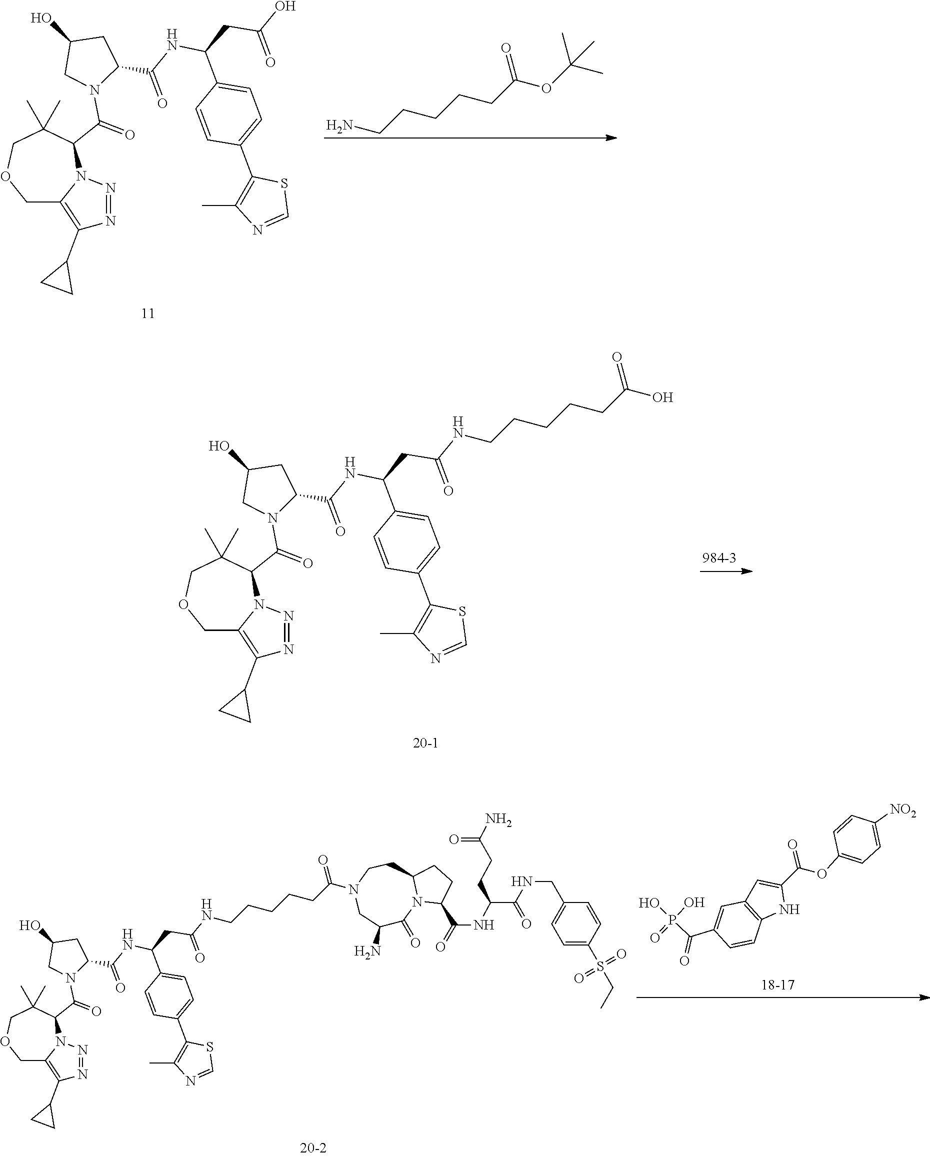 [SMILES: Cc1ncsc1-c1ccc([C@H](CC(=O)O)NC(=O)[C@H]2C[C@H](O)CN2C(=O)[C@H]2n3nnc(C4CC4)c3COCC2(C)C)cc1 ; CC(C)(C)OC(=O)CCCCCN ; Cc1ncsc1-c1ccc([C@H](CC(=O)NCCCCCC(=O)O)NC(=O)[C@H]2C[C@H](O)CN2C(=O)[C@H]2n3nnc(C4CC4)c3COCC2(C)C)cc1 ; CCS(=O)(=O)c1ccc(CNC(=O)[C@H](CCC(N)=O)NC(=O)[C@@H]2CC[C@@H]3CCN(C(=O)CCCCCNC(=O)C[C@H](NC(=O)[C@H]4C[C@H](O)CN4C(=O)[C@H]4n5nnc(C6CC6)c5COCC4(C)C)c4ccc(-c5scnc5C)cc4)C[C@H](N)C(=O)N32)cc1 ; O=C(Oc1ccc([N+](=O)[O-])cc1)c1cc2cc(C(=O)P(=O)(O)O)ccc2[nH]1]

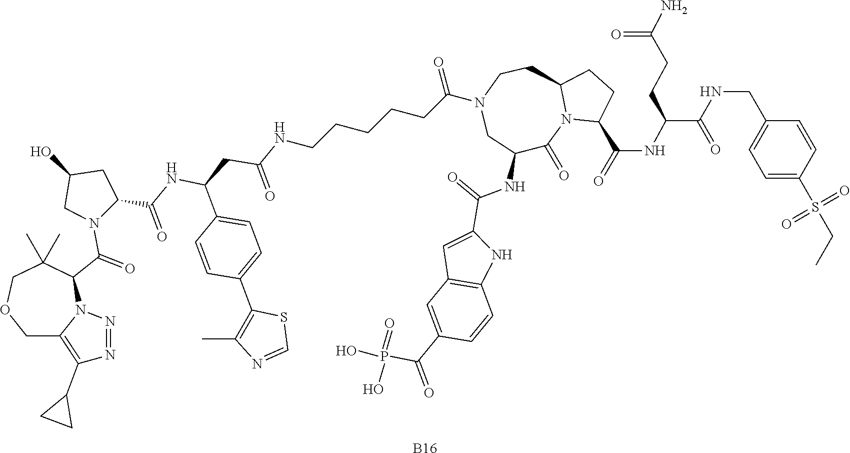

984-1: HATU (8.5 g, 22.3 mmol, 1.1 equiv.) was added to a solution of the Boc-Gin-OH (20.3 mmol, 1 equiv.), (4-(ethylsulfonyl)phenyl)methanamine (20.3 mmol, 1 equiv.) and DIEA (10.6 mL, 60.9 mmol, 3 equiv.) in DMF (60 mL) and the resulting mixture was stirred at rt for 1 h. The solution was diluted with EtOAc and washed with H120, saturated sodium bicarbonate aqueous solution and brine, and dried over sodium sulfate. After removal of the solvent in vacuo, the residue was purified by flash chromatography on silica gel to afford 984-1. UPLC-MS calculated [M+H]⁺: 428.2, found: 428.7.

984-2: TFA (5 mL) was added slowly to a solution of 984-1 (3 g) in DCM (50 mL) and the resulting reaction solution was stirred at rt for 6 h and then evaporated. The residue was used directly in the next step without further purification. UPLC-MS calculated [M+H]⁺: 328.1, found: 328.5.

984-3: HATU (0.55 mmol, 1.1 equiv.) was added to a solution of the 984-2 (0.5 mmol, 1 equiv.), 8 (0.5 mmol, 1 equiv.) and DIEA (1.5 mmol, 3 equiv.) in DMF (60 mL) and the resulting mixture was stirred at rt for 1 h. The solution was diluted with EtOAc and washed with H120, saturated sodium bicarbonate aqueous solution and brine, and dried over sodium sulfate. After removal of the solvent in vacuo, the residue was purified by flash chromatography on silica gel to afford 9. The obtained product was dissolved in Acetonitrile (10 mL) and Diethylamine (0.5 mL) was added to the solution. The resulting mixture was stirred at rt for 20 min and the solvent was removed under vacuum. The residue was purified by HPLC to yield 984-3. UPLC-MS calculated [M+H]⁺: 637.3, found: 637.5.

20-1: HATU (0.11 mmol, 1.1 equiv.) was added to a solution of the 11 (0.1 mmol, 1 equiv.), tert-butyl 6-aminohexanoate (0.1 mmol, 1 equiv.) and DIEA (0.3 mmol, 3 equiv.) in DMF (1 mL) and the resulting mixture was stirred at rt for 1 h. The solution was diluted with EtOAc and washed with H₂O, saturated sodium bicarbonate aqueous solution and brine, and dried over sodium sulfate. After removal of the solvent in vacuo, the residue was treated with 1 mL of 1M HCl (in Methanol) and 1 mL of 4M HCl (in dioxane) for 12 h. After evaporation, the residue was used directly in the next step without further purification. UPLC-MS calculated [M+H]⁺: 722.3, found: 722.6.

20-2: HATU (0.11 mmol, 1.1 equiv.) was added to a solution of 984-3 (0.1 mmol, 1 equiv.), 20-1 (0.1 mmol, 1 equiv.) and DIEA (0.3 mmol, 3 equiv.) in DMF (1 mL) and the resulting mixture was stirred at rt for 1 h. This mixture was purified by reverse phase preparative HPLC. The obtained product was treated with 1 mL of 1M HCl (in Methanol) and 1 mL of 4M HCl (in dioxane) for 1 h. The reaction mixture was evaporated to give the 20-2 without further purification. UPLC-MS calculated [M+2H]²⁺: 620.8, found: 620.9.

A16: DIEA (0.068 mmol, 3 equiv.) was added to the mixture of compound 18-17 (0.081 mmol, 1.2 equiv.), compound 20-2 (0.069 mmol, 1 equiv.) and HOBt (0.14 mmol, 2 equiv.) in DMF (1 mL). The resulted mixture was stirred at room temperature for 1 hour. Purification of this reaction mixture by HPLC gave the compound B16. UPLC-MS calculated [M+2H]²⁺: 746.3, found: 746.5.

### Compound B17. (2-(((5S,8S,10aR)-3-acetyl-8-(((S)-5-amino-1-(2-chloro-3-(3-(1-((R)-2-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-2-(4-(4-methylthiazol-5-yl)phenyl)ethyl)-1H-1,2,3-triazol-4-yl)propyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

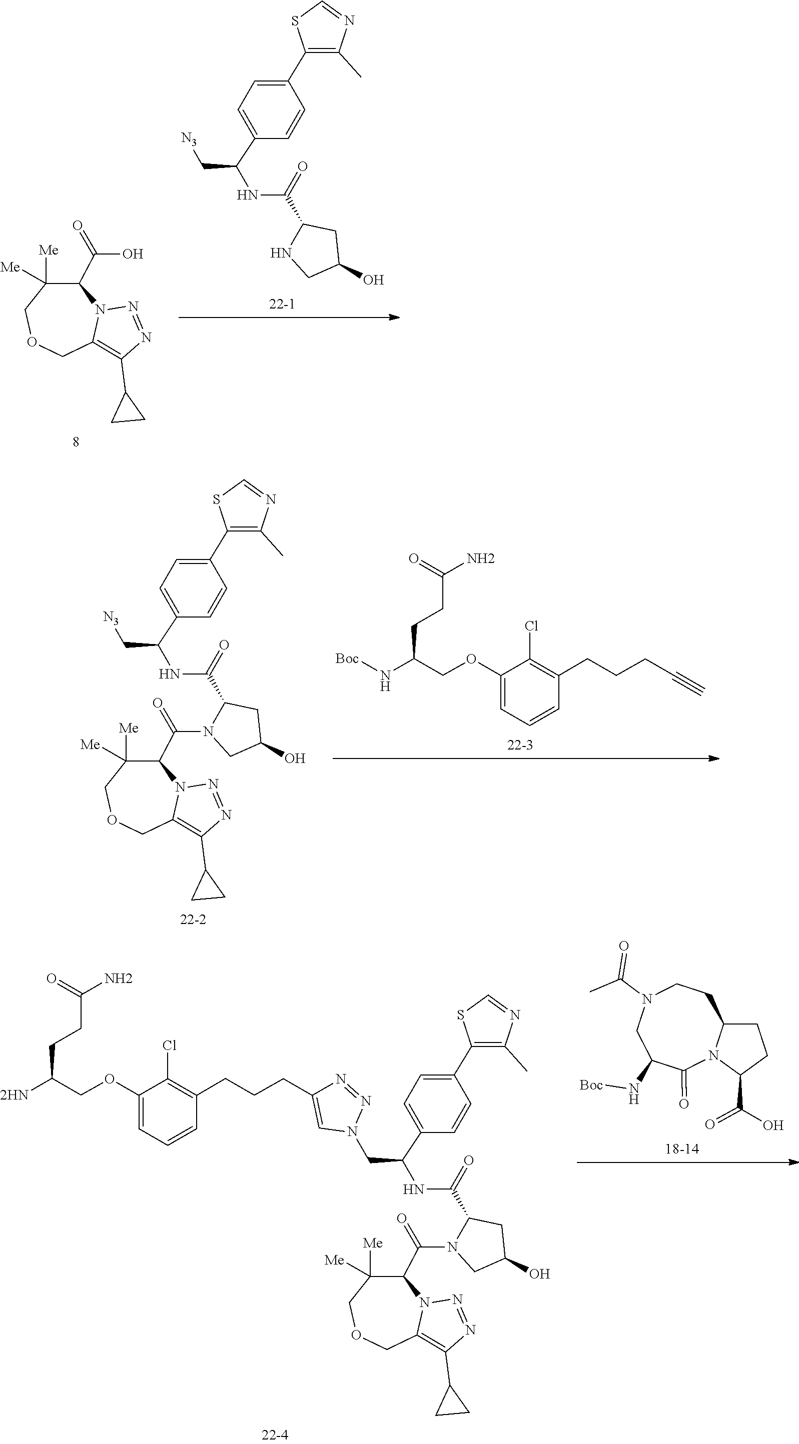

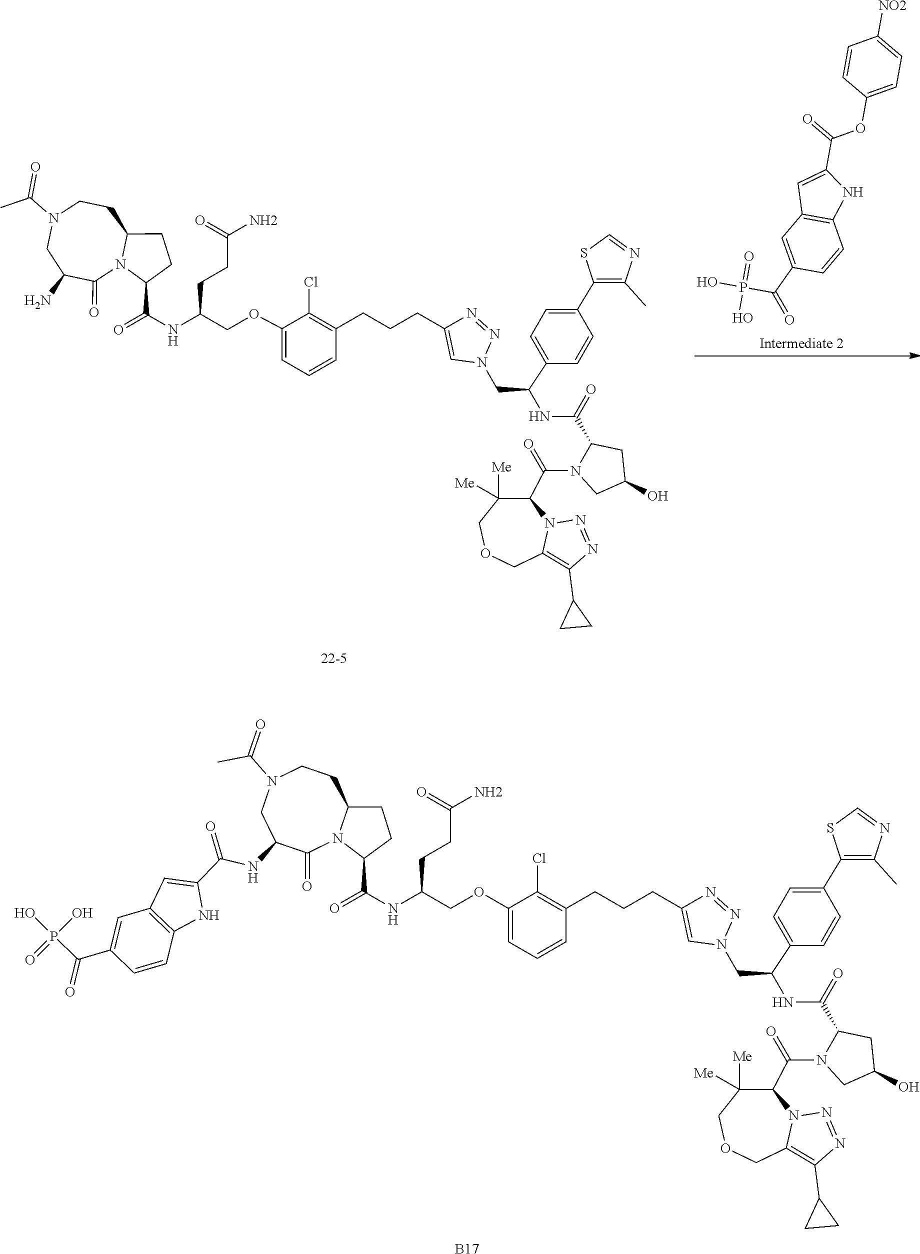 [SMILES: CC(=O)N1CC[C@H]2CC[C@@H](C(=O)N[C@@H](CCC(N)=O)COc3cccc(CCCc4cn(C[C@H](NC(=O)[C@@H]5C[C@@H](O)CN5C(=O)[C@H]5n6nnc(C7CC7)c6COCC5(C)C)c5ccc(-c6scnc6C)cc5)nn4)c3Cl)N2C(=O)[C@@H](N)C1 ; O=C(Oc1ccc([N+](=O)[O-])cc1)c1cc2cc(C(=O)P(=O)(O)O)ccc2[nH]1 ; CC(=O)N1CC[C@H]2CC[C@@H](C(=O)N[C@@H](CCC(N)=O)COc3cccc(CCCc4cn(C[C@H](NC(=O)[C@@H]5C[C@@H](O)CN5C(=O)[C@H]5n6nnc(C7CC7)c6COCC5(C)C)c5ccc(-c6scnc6C)cc5)nn4)c3Cl)N2C(=O)[C@@H](NC(=O)c2cc3cc(C(=O)P(=O)(O)O)ccc3[nH]2)C1 ; "B17"]

122-2: HATU (0.08 mmol, 1.1 equiv.) was added to a solution of 8 (0.07 mmol, 1 equiv.), 22-1 (0.07 mmol, 1 equiv.) and DIEA (0.21 mmol, 3 equiv.) in DMF (1 mL) and the resulting mixture was stirred at rt for 1 h. This mixture was purified by reverse phase preparative HPLC to give 22-2. UPLC-MS calculated [M+H]+: 606.3, found: 606.4. Retention time: 4.2 m.

122-4: 22-2 (0.05 mmol), Copper sulfate (0.06 mmol) and 22-3 (0.05 mmol) were added to a solution sodium L-ascorbate (0.15 mmol) in water (1 mL) and tert-Butyl alcohol (2 mL). The reaction was stirred at room temperature for 5 h and extracted with ethyl acetate. The combined organic layers were concentrated under reduced pressure and the residue was purified by RP-HPLC. The obtained product was treated with 1 ml, of 1M HCl (in Methanol) and 1 ml, of 4M HCl (in dioxane) for 1 h. The reaction mixture was evaporated to give the 22-4 without further purification. UPLC-MS calculated [M+H]P: 914.4, found: 914.6.

22-5: HATU (0.022 mmol, 1.1 equiv.) was added to a solution of 22-4 (0.02 mmol, 1 equiv.), 18-14 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC. The obtained product was treated with 1 ml, of 1M HCl (in Methanol) and 1 ml, of 4M HCl (in dioxane) for 1 h. The reaction mixture was evaporated to give the 22-5 without further purification. UPLC-MS calculated [M+H]+: 1165.5, found: 1165.8.

B17. DIEA (0.042 mmol, 3 equiv.) was added to the mixture of Intermediate 2 (0.017 mmol, 1.2 equiv.), compound 22-5 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B17. UPLC-MS calculated [M+2H]²⁺; 708.8, found: 708.9. Retention time: 3.4 min.

### Compound B18. (2-(((5S,8S,10aR)-3-acetyl-8-(((S)-5-amino-1-(2-chloro-3-(4-((S)-3-((2S,4R)-1-((S)-3-cyclopropyl-7,7-dimethyl-7,8-dihydro-4H,6H-[1,2,3]triazolo[5,1-c][1,4]oxazepine-8-carbonyl)-4-hydroxypyrrolidine-2-carboxamido)-3-(4-(4-methylthiazol-5-yl)phenyl)propanamido)butyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

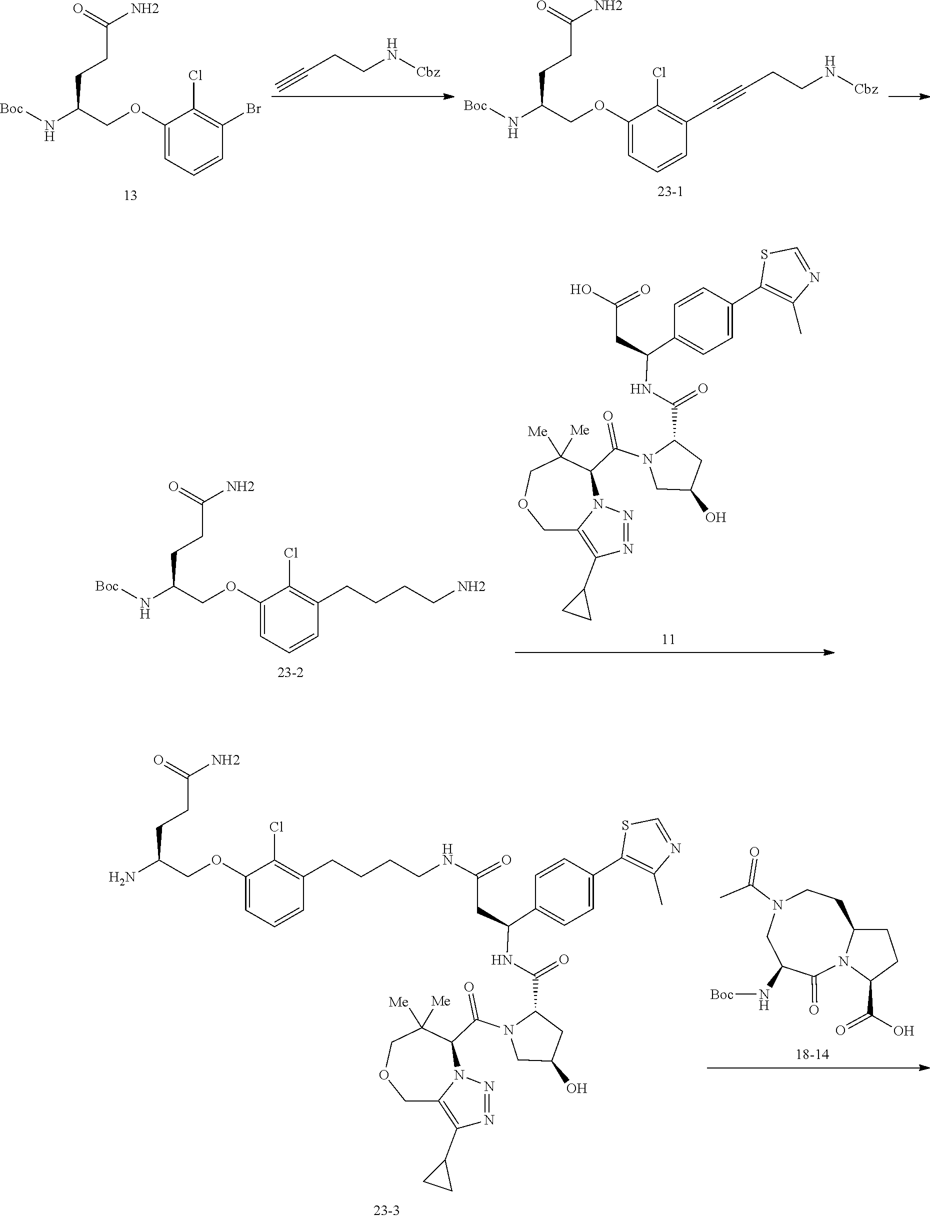

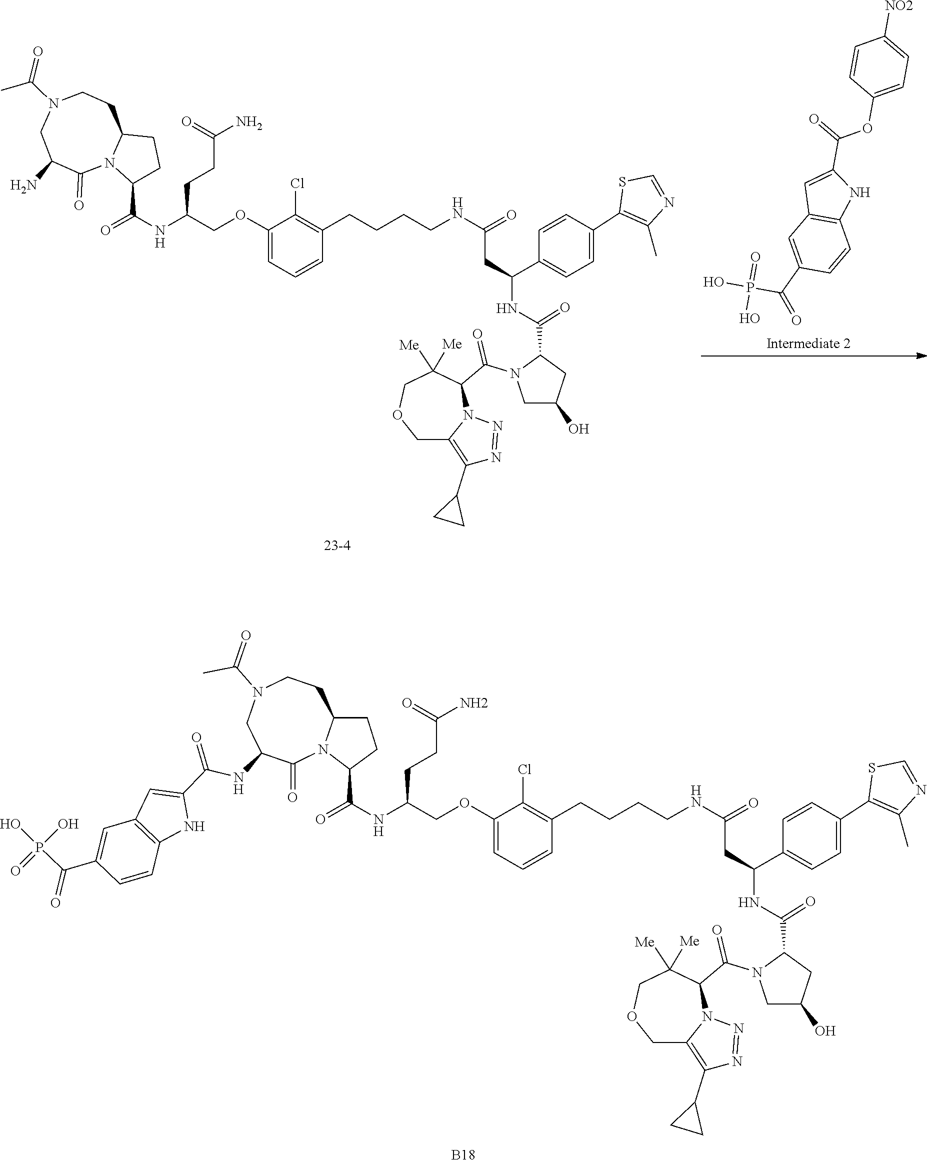 [SMILES: CC(=O)N1CC[C@H]2CC[C@@H](C(=O)N[C@@H](CCC(N)=O)COc3cccc(CCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](N)C1 ; O=C(Oc1ccc([N+](=O)[O-])cc1)c1cc2cc(C(=O)P(=O)(O)O)ccc2[nH]1 ; CC(=O)N1CC[C@H]2CC[C@@H](C(=O)N[C@@H](CCC(N)=O)COc3cccc(CCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1 ; "B18"]

23-1: Trimethylamine (3 mL) was added to a mixture of compound 13 (1.0 mmol, 1 equiv.), benzyl but-3-yn-1-ylcarbamate (1.5 mmol, 1.5 equiv.), CuI (154 mg, 0.2 mmol, 0.2 equiv) and Pd(PPh3)2Cl2 (282 mg, 0.1 mmol, 0.1 equiv) in DMF (5 mL). The resulting mixture was purged and refilled with argon three times and stirred at 70-80° C. for 3 h under Argon. The reaction mixture was then cooled to room temperature and evaporated to remove most of the solvent. The residue was purified by HPLC to yield the 23-1. UPLC-MS calculated [M+H]+: 544.2, found: 544.6.

23-2: To a solution of compound 23-1 (0.1 mmol) in MeOH (4 mL) was added platinum(iv) oxide (15 mg). The mixture was stirred under 1 atm of H₂ at room temperature for 3 hours before filtering through celite and being concentrated. The resulting amine 23-2 was used for the next step without further purification. UPLC-MS calculated [M+H]⁺: 414.2, found: 414.7.

23-3: HATU (0.022 mmol, 1.1 equiv.) was added to a solution of 11 (0.02 mmol, 1 equiv.), 23-2 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC. The obtained product was treated with 1 mL of 1M HCl (in Methanol) and 1 mL of 4M HCl (in dioxane) for 1 h. The reaction mixture was evaporated to give the 23-3 without further purification. UPLC-MS calculated [M+H]+: 904.4, found: 904.9.

23-4: HATU (0.022 mmol, 1.1 equiv.) was added to a solution of 23-3 (0.02 mmol, 1 equiv.), 18-14 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC. The obtained product was treated with 1 mL of 1M HCl (in Methanol) and 1 mL of 4M HCl (in dioxane) for 1 h. The reaction mixture was evaporated to give the 23-4 without further purification. UPLC-MS calculated [M+H]+: 1155.5, found: 1155.8.

DIEA (0.042 mmol, 3 equiv.) was added to the mixture of Intermediate 2 (0.017 mmol, 1.2 equiv.), compound 23-4 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B18. UPLC-MS calculated [M+2H]²⁺: 703.8, found: 703.8. Retention time: 3.4 min.

### Compound B19. (2-(((5S,8S,10aR)-3-acetyl-8-(((S)-5-amino-1-(2-chloro-3-(3-((S)-8-((2S,4R)-4-hydroxy-2-(((R)-2-hydroxy-1-(4-(4-methylthiazol-5-yl)phenyl)ethyl)carbamoyl)pyrrolidine-1-carbonyl)-7,7-dimethyl-7,8-dihydro-4H,6H-[1,2,3]triazolo[5,1-c][1,4]oxazepin-3-yl)propyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

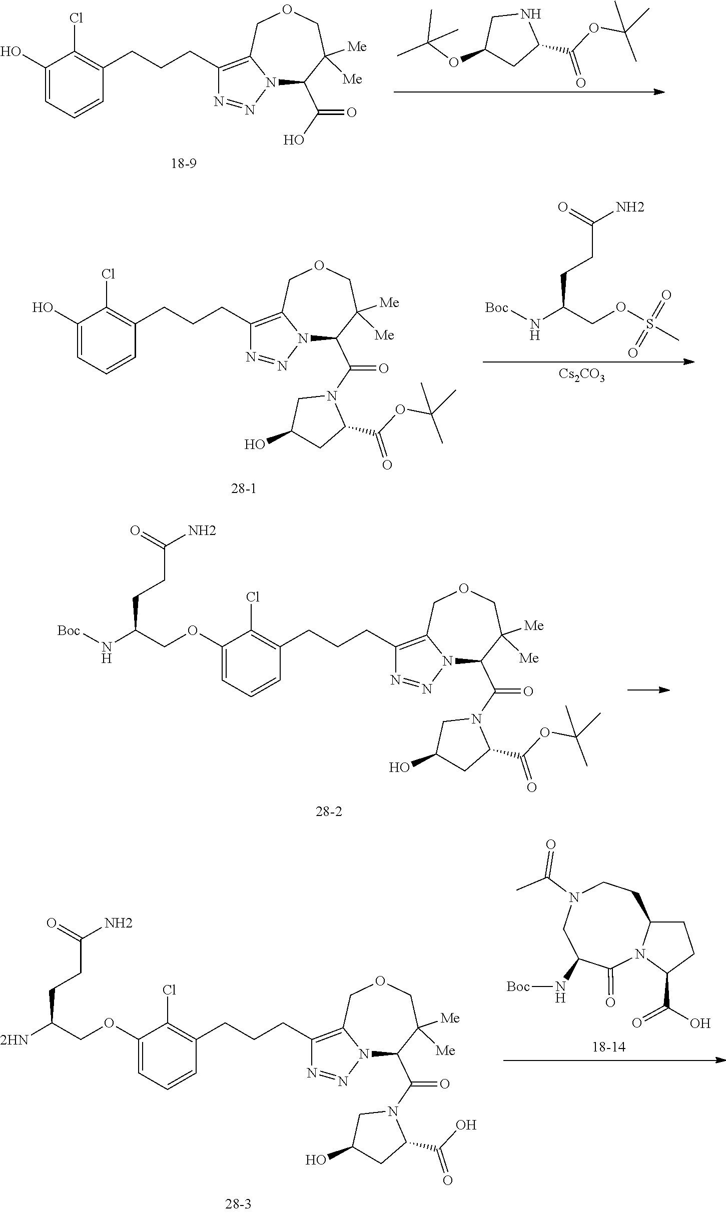

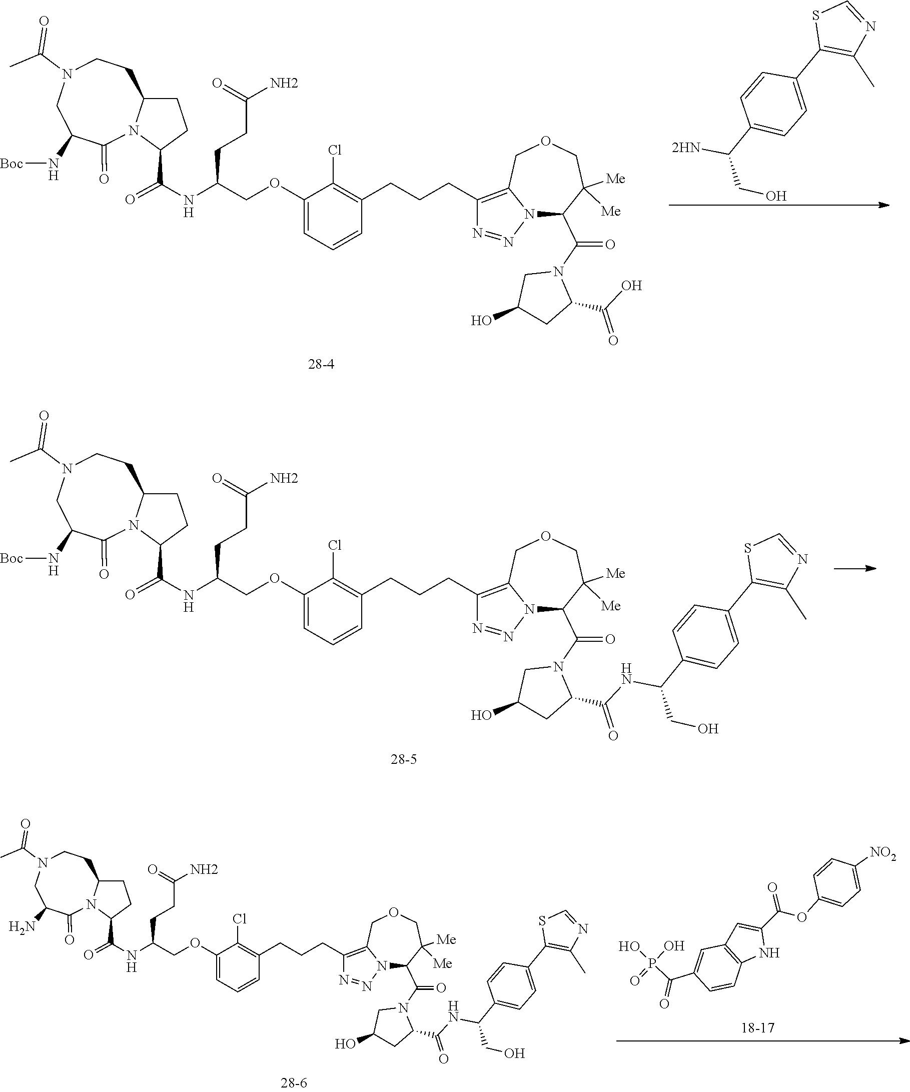

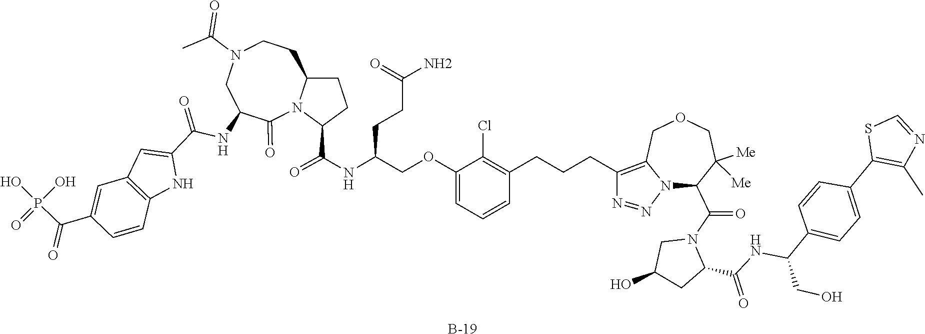

28-1: HATU (0.11 mmol, 1.3 equiv.) was added to a solution of 18-9 (33 mg, 0.087 mmol, 1 equiv.), tert-butyl (2S,4R)-4-(tert-butoxy)pyrrolidine-2-carboxylate (0.087 mmol, 1 equiv.) and DIEA (0.26 mmol, 3 equiv.) in DMF (2 mL) and the resulting mixture was stirred at rt for 1 h. Then 5 mL of MeOH was added to this reaction mixture and stirred for 2 hours. After removing most of MeOH under reduced pressure, the residue was purified by reverse phase preparative HPLC to give 28-1. UPLC-MS calculated [M+H]⁺: 605.3, found: 605.6. Retention time: 6.3 min.

28-2: NaI (0.1 mmol, 0.5 equiv.) and CsCO₃ (0.4 mmol, 2 equiv.) were added to a stirred solution of 28-1 (0.2 mmol, 1 equiv.) and (S)-5-amino-2-((tert-butoxycarbonyl)amino)-5-oxopentyl methanesulfonate (0.4 mmol, 2 equiv.) in DMF (1 mL) at rt under nitrogen atmosphere. The resulting mixture was stirred for overnight at 55° C. Additional of NaI (0.1 mmol, 0.5 equiv.), CsCO₃ (0.4 mmol, 2 equiv.) and (S)-5-amino-2-((tert-butoxycarbonyl)amino)-5-oxopentyl methanesulfonate (0.4 mmol, 2 equiv.) were added to the reaction mixture and this resulting mixture was stirred at 55 C for 5 h. This mixture was purified by reverse phase preparative HPLC to yield 28-2. UPLC-MS calculated [M+H]⁺: 819.4.3, found: 819.4. Retention time: 7.1 min.

28-3: Compound 28-2 (95 mg) was treated with 5 mL of 4M HCl (in dioxane) for 3 h. The reaction mixture was evaporated to give the 28-3 without further purification. UPLC-MS calculated [M+H]⁺: 607.3, found: 607.3. Retention time: 1.7 min.

28-4: HATU (0.022 mmol, 1.1 equiv.) was added to a solution 18-14 (0.02 mmol, 1 equiv.) in DMF (1 mL) and the resulting mixture was stirred at rt for 20 min. Then DIEA (0.06 mmol, 3 equiv.) and 28-3 were added and the resulting mixture was stirred at rt for 1 h. This mixture was purified by reverse phase preparative HPLC to yield 28-4. UPLC-MS calculated [M+H]⁺: 958.4, found: 958.7. Retention time: 3.8 min.

28-5: HATU (0.11 mmol, 1.3 equiv.) was added to a solution of 28-4 (33 mg, 0.087 mmol, 1 equiv.), (R)-2-amino-2-(4-(4-methylthiazol-5-yl)phenyl)ethan-1-ol (0.087 mmol, 1 equiv.) and DIEA (0.26 mmol, 3 equiv.) in DMF (2 mL) and the resulting mixture was stirred at rt for 1 h. This mixture was purified by reverse phase preparative HPLC to give 28-5. UPLC-MS calculated [M+H]⁺: 1174.5, found: 1174.9. Retention time: 3.6 min.

28-6: Compound 28-5 (20 mg) was treated with 1 mL of 1M HCl (in Methanol) and 1 mL of 4M HCl (in dioxane) for 2 h. The reaction mixture was evaporated to give the 28-6 without further purification. UPLC-MS calculated [M+H]⁺: 1074.5, found: 1074.8

B19: DIEA (0.042 mmol, 3 equiv.) was added to the mixture of 18-17 (0.017 mmol, 1.2 equiv.), compound 28-6 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B19. UPLC-MS calculated [M+2H]²⁺: 663.2, found: 663.3. Retention time: 3.0 min.

### Compound B20. (2-(((5S,8S,10aR)-3-acetyl-8-(((S)-5-amino-1-(2-chloro-3-(3-((S)-8-((2S,4R)-4-hydroxy-2-(((R)-1-(4-(4-methylthiazol-5-yl)phenyl)-2-morpholinoethyl)carbamoyl)pyrrolidine-1-carbonyl)-7,7-dimethyl-7,8-dihydro-4H,6H-[1,2,3]triazolo[5,1-c][1,4]oxazepin-3-yl)propyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

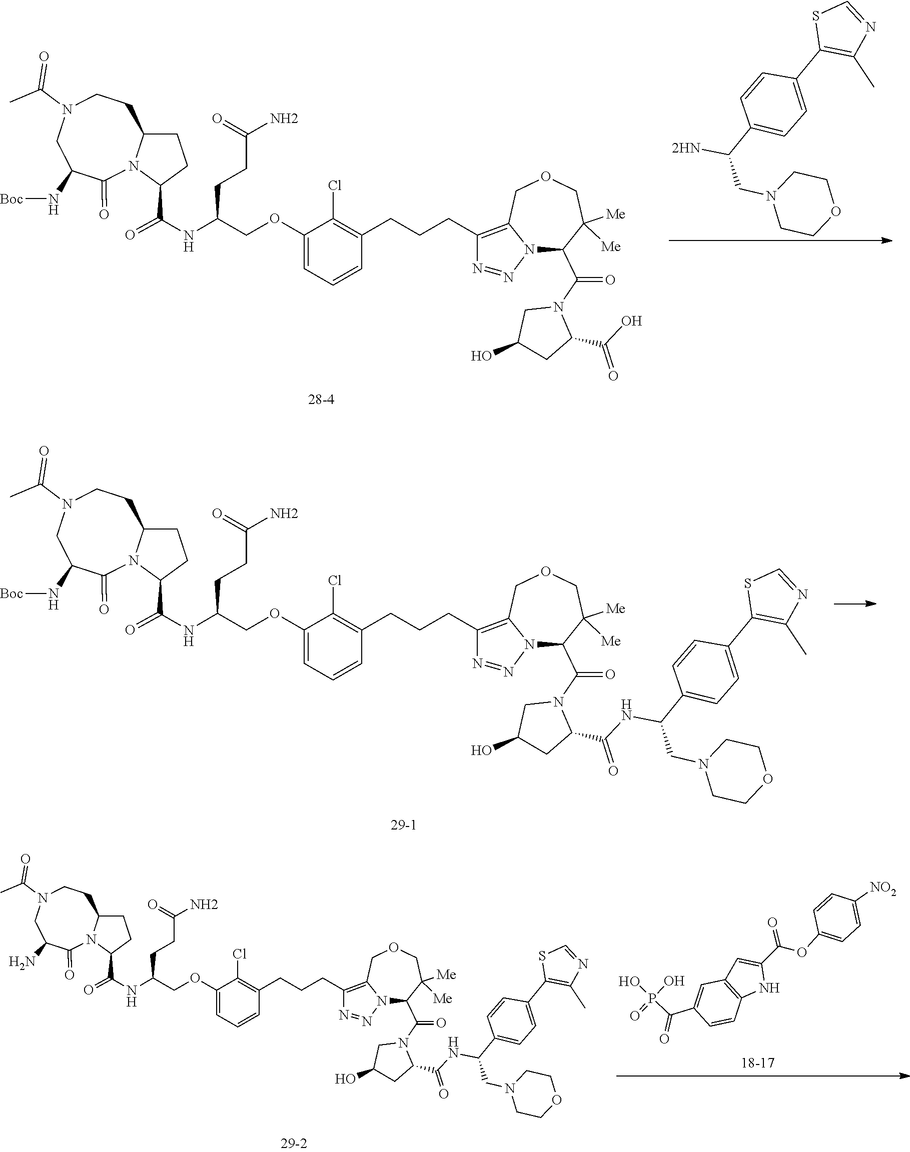

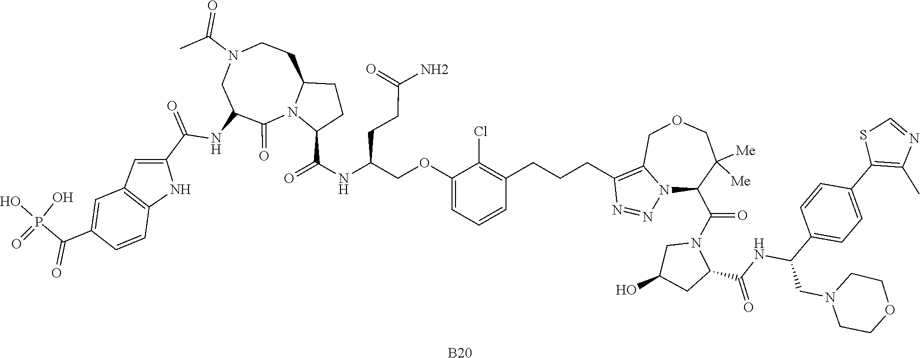

29-1: HATU (0.11 mmol, 1.3 equiv.) was added to a solution of 28-4 (33 mg, 0.087 mmol, 1 equiv.), (R)-1-(4-(4-methylthiazol-5-yl)phenyl)-2-morpholinoethan-1-amine (0.087 mmol, 1 equiv.) and DIEA (0.26 mmol, 3 equiv.) in DMF (2 mL) and the resulting mixture was stirred at rt for 1 h. This mixture was purified by reverse phase preparative HPLC to give 29-1. UPLC-MS calculated [M+2H]²⁺: 622.3, found: 622.3.

29-2: Compound 29-1 (20 mg) was treated with 1 mL of 1M HCl (in Methanol) and 1 mL of 4M HCl (in dioxane) for 2 h. The reaction mixture was evaporated to give the 29-2 without further purification. UPLC-MS calculated [M+H]⁺: 1142.5, found: 1142.7

B20: DIEA (0.042 mmol, 3 equiv.) was added to the mixture of 18-17 (0.017 mmol, 1.2 equiv.), compound 29-2 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B20. UPLC-MS calculated [M+2H]²⁺: 697.8, found: 697.6. Retention time: 2.8 min.

### Compound B21. (2-(((5S,8S,10aR)-3-acetyl-8-(((S)-5-amino-1-(2-chloro-3-(5-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-5,6,7,8-tetrahydro-4H-cyclohepta[d]isoxazole-3-carboxamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

### Compound B22. ((2-(((5S,8S,10aR)-3-acetyl-8-(((S)-5-amino-1-(2-chloro-3-(5-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-5,6,7,8-tetrahydro-4H-cyclohepta[d]isoxazole-3-carboxamido)pentyl)phenoxy)-5-oxopentan-2-yl)carbamoyl)-6-oxodecahydropyrrolo[1,2-a][1,5]diazocin-5-yl)carbamoyl)benzo[b]thiophen-5-yl)difluoromethyl)phosphonic acid

### Compound B26. (2-(((3S,6S,10aS)-3-(((S)-5-amino-1-(2-chloro-3-(5-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-5,6,7,8-tetrahydro-4H-cyclohepta[d]isoxazole-3-carboxamido)pentyl)phenoxy)-5-oxopentan-2-yl)carbamoyl)-5-oxodecahydropyrrolo[1,2-a]azocin-6-yl)carbamoyl)-1H-indole-5-carbonyl)phosphonic acid

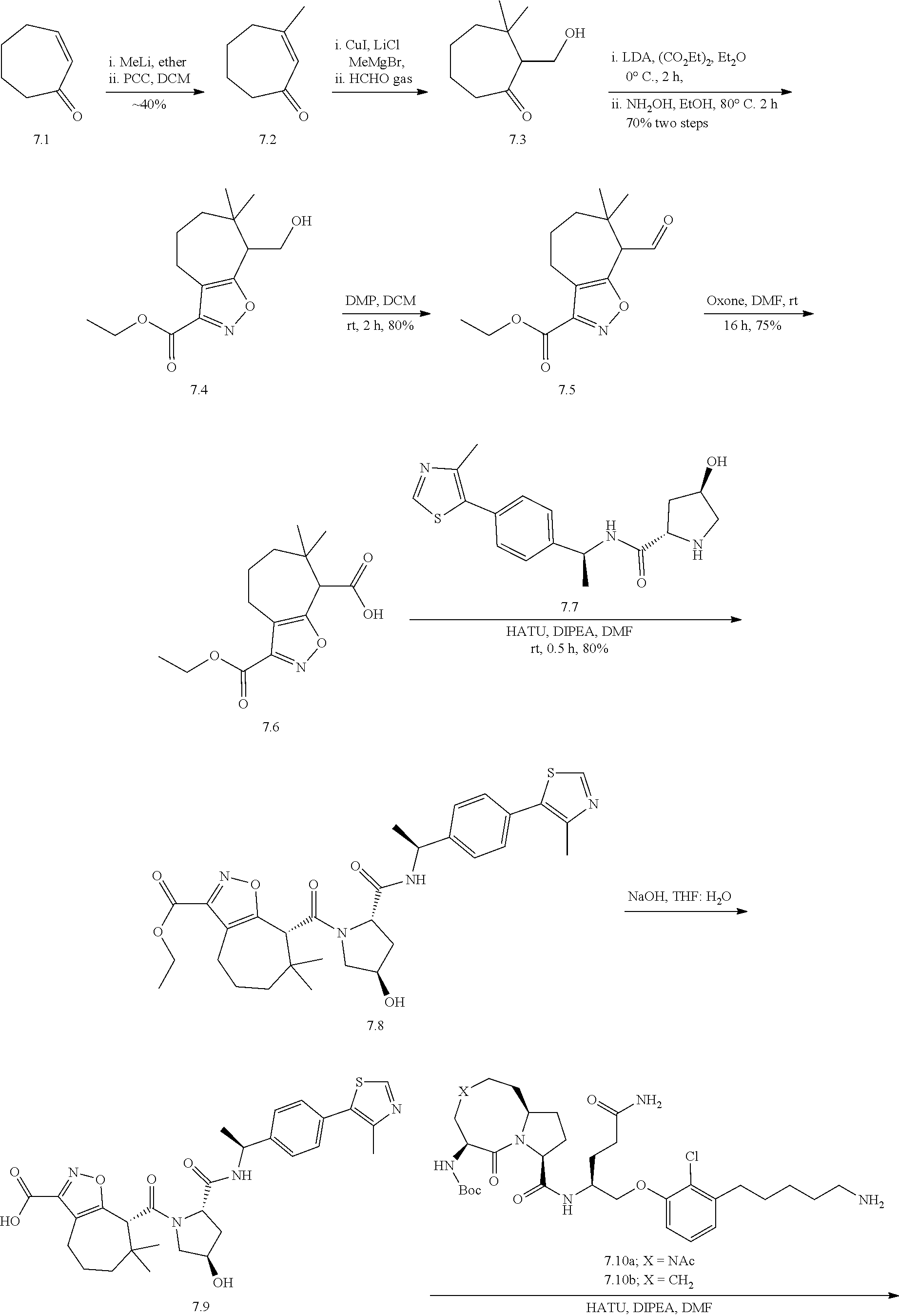

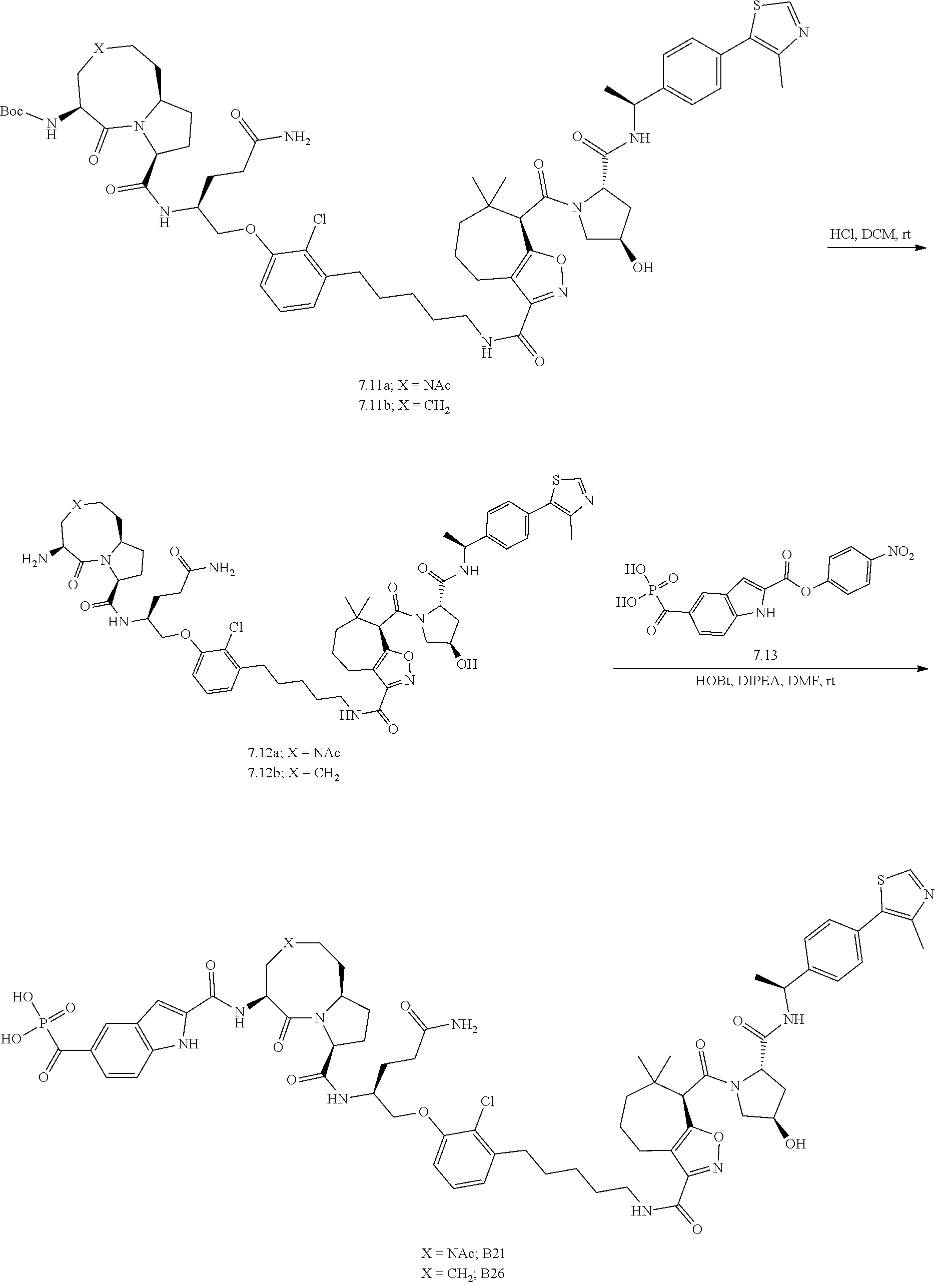

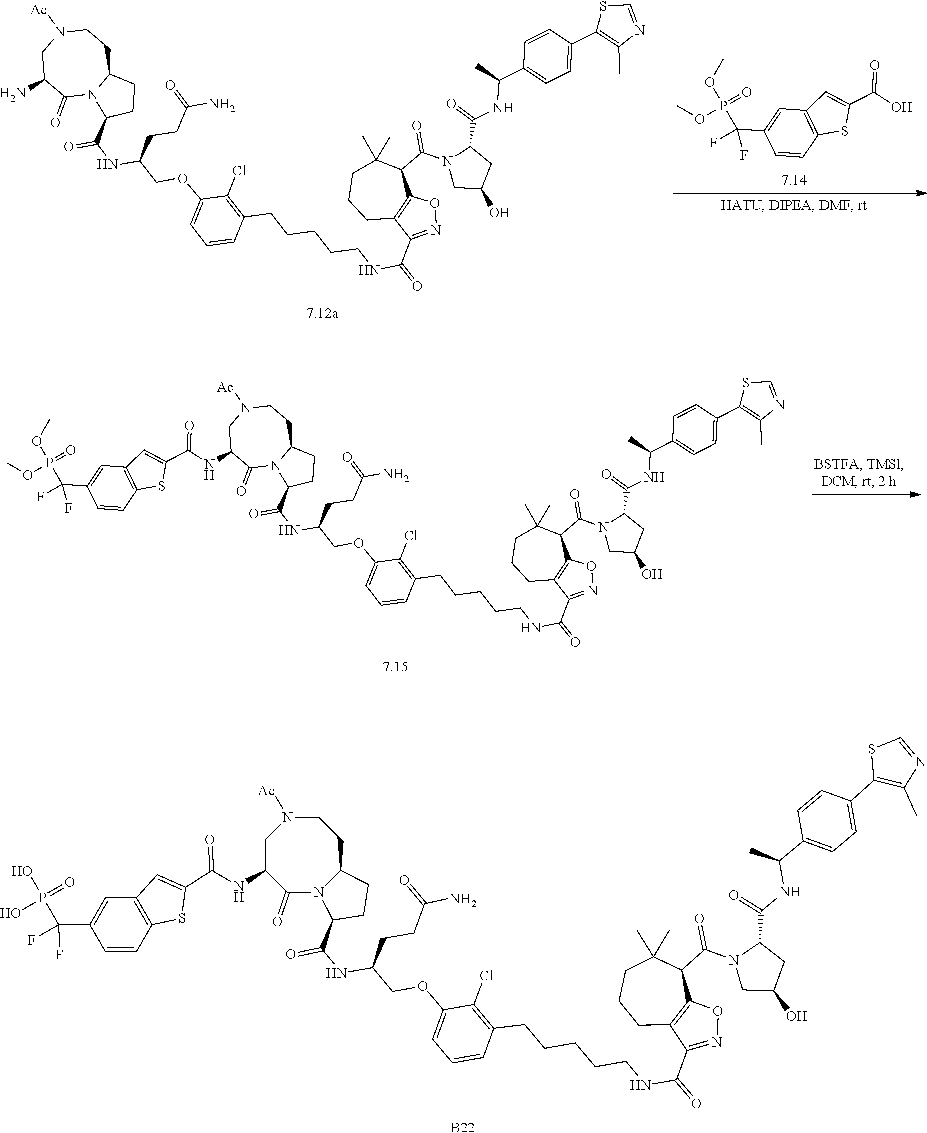

### Step 1

To a stirred solution of compound 7.1 in diethyl ether at −78° C., MeLi (1.1 eq) was added dropwise, and the reaction was stirred at the same temperature for another 2 h. After complete conversion of the starting material, the reaction was quenched with saturated ammonium chloride and extracted with diethyl ether. The organic layer was washed with brine and dried over Na₂SO₄. The crude product was used for the next step.

To a stirred solution of the crude product in DCM at rt, PCC (1.5 eq) was added, and the reaction was vigorously stirred for 2 h. The reaction was monitored by TLC chromatography. After complete conversion of the starting material, the reaction was diluted with ethyl ether and filtered through a pad of celite. The mixture was washed with ethyl ether. The filtrate was concentrated under reduced pressure and the product was purified with flash column chromatography. ¹H NMR (400 MHz, CDCl₃) δ 5.95 (d, J=1.9 Hz, 1H), 2.67-2.52 (m, 2H), 2.43 (dd, J=6.9, 4.6 Hz, 2H), 1.98 (dd, J=1.3, 0.7 Hz, 3H), 1.90-1.73 (m, 4H).

### Step 2

LiCl (0.1 eq) and CuI (0.05 eq) were added to a flame-dried two-way round bottom flask and purged with nitrogen three times. THF was added to the mixture and stirred vigorously until a homogeneous light-yellow solution. Compound 7.2 was added, and the reaction was cooled to 0° C. Methyl magnesium bromide (1.2 eq) was added to the reaction dropwise over 20 mins and the reaction was stirred at the same temperature for 2 h and monitored by TLC chromatography. After the complete conversion of starting material, the reaction was cooled to −10° C., and formaldehyde gas (generated by heating paraformaldehyde at 140° C.) was passed through the solution using a cannula for 4 h. The reaction was monitored by TLC chromatography. Upon completion of the reaction, the mixture was quenched with a saturated solution of ammonium chloride and extracted with ethyl acetate. The product was purified by flash column chromatography. ¹H NMR (400 MHz, CDCl₃) δ 4.02 (dd, J=11.2, 9.0 Hz, 1H), 3.71 (dd, J=11.2, 3.6 Hz, 1H), 2.96 (dd, J=9.0, 3.6 Hz, 1H), 2.67 (s, 2H), 2.59-2.34 (m, 2H), 1.89-1.73 (m, 3H), 1.66-1.40 (m, 2H), 1.05 (s, 3H), 0.83 (s, 3H).

### Step 3

A solution of compound 7.3 (1 eq), diethyl oxalate (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

A solution of crude product and hydroxyl amine hydrochloride (2 eq) in ethanol was heated to reflux for 2 h. After complete conversion of starting material, ethanol was evaporated under reduced pressure and the product was purified with flash column chromatography. ¹H NMR (400 MHz, CDCl₃) δ 4.41 (q, J=7.1 Hz, 2H), 3.81 (hept, J=6.7 Hz, 1H), 3.43 (hept, J=6.8 Hz, 1H), 3.14-3.03 (m, 1H), 2.98 (ddd, J=7.1, 5.9, 1.1 Hz, 1H), 2.60-2.44 (m, 1H), 2.06 (s, 2H), 1.88 (ddd, J=14.4, 11.1, 3.3 Hz, 1H), 1.71 (ddt, J=7.1, 6.0, 3.6 Hz, 1H), 1.61-1.53 (m, 1H), 1.40 (t, J=7.2 Hz, 3H), 1.05 (s, 3H), 0.93 (s, 3H). UPLC-MS: calculated [M+H]⁺: 268.15, found: 268.17.

### Step 4

To a stirred solution of compound 7.4 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography. ¹H NMR (400 MHz, CDCl₃) δ 9.85 (d, J=2.4 Hz, 1H), 4.41 (q, J=7.1 Hz, 2H), 3.76 (dd, J=2.4, 1.4 Hz, 1H), 3.16 (dddd, J=16.0, 5.2, 3.1, 1.2 Hz, 1H), 2.60-2.47 (m, 1H), 1.89 (ddd, J=14.0, 11.5, 2.7 Hz, 1H), 1.83-1.63 (m, 3H), 1.40 (t, J=7.1 Hz, 3H), 1.27 (s, 3H), 0.98 (s, 3H). UPLC-MS: calculated [M+H]⁺: 266.13, found: 266.07.

### Step 5

To a stirred solution of compound 7.5 (1 eq) in DMF at rt, Oxone monohydrate (3 eq) was added. The reaction was stirred at rt for 16 h followed by dilution with water. The mixture was extracted with ethyl acetate and the product was purified using flash column chromatography. UPLC-MS: calculated [M+H]⁺: 282.13, found: 282.04.

### Step 6

To a stirred solution of compound 7.6 (1 eq), compound 7.7 (1 eq), and HATU (1 eq) in DMF at rt, DIPEA (1.5 eq) was added dropwise and monitored by UPLC. After completion of the reaction, the product was purified by reverse phase column chromatography. UPLC-MS: calculated [M+H]⁺: 594.25, found: 595.10.

### Step 7

To a stirred solution of compound 7.8 in THF:water (1:1) at rt, NaOH was added and the reaction was stirred for 2 h. After complete conversion of the starting material, it was acidified with 2 N HCl up to pH 3 and extracted with ethyl acetate. The product was purified with flash column chromatography. UPLC-MS: calculated [M+H]⁺: 567.22, found: 567.12.

### Step 8

To a stirred solution of compound 7.9 (1 eq), compound 7.10a (1 eq), and HATU (1 eq) in DMF at rt, DIPEA (1.5 eq) was added dropwise and monitored by UPLC. After completion of the reaction, the product was purified by preparative HPLC (ACN: H₂O with 0.1% TFA). UPLC-MS: calculated [M+2H]²⁺/2: 614.28, found: 614.15.

### Step 9

To a stirred solution of 7.11a in DCM, 4 N HCl in dioxane was added and stirred for 1 h at room temperature and monitored by UPLC. After completion of the reaction solvent was evaporated to dryness and the crude product was used for the next step. UPLC-MS: calculated [M+H]⁺: 1127.51, found: 1127.15.

### Step 10

To a stirred solution of compound 7.12a (1 eq), compound 7.13 (1 eq), and HOBt (1 eq) in DMF at rt, DIPEA (1.5 eq) was added dropwise and monitored by UPLC. After completion of the reaction, the product was purified by preparative HPLC (ACN: H₂O with 0.1% TFA) to get B21 as white solid. UPLC-MS: calculated [M+2H]²+/2: 689.5, found: 689.9.

B26 was made following similar synthetic procedure described earlier. UPLC-MS: calculated [M+2H]²⁺/2: 668.25, found: 668.50.

To a stirred solution of compound 7.12a (1 eq), compound 7.14 (1 eq), and HATU (1 eq) in DMF (2 mL) at rt, DIPEA (1.5 eq) was added dropwise and monitored by UPLC. After completion of the reaction, the product was purified by preparative HPLC (ACN: H₂O with 0.1% TFA) to get 7.15 as white solid. UPLC-MS: calculated [M+2H]²+/2: 723.25, found: 723.5.

To a stirred solution of compound 7.15 (1 eq) in DCM (2 mL) at ° C., BSTFA (0.1 eq) and TMSI (1.5 eq) was added dropwise and monitored by UPLC. After completion of the reaction, the product was purified by preparative HPLC (ACN: H₂O with 0.1% TFA) to get B22 as white solid. UPLC-MS: calculated [M+2H]²+/2: 709.25, found: 709.5.

### Compound B25. (2-(((5S,8S,10aR)-3-acetyl-8-(((S)-5-amino-1-(2-chloro-3-(5-((R)-7-((2S,4R)-4-hydroxy-2-(((S)-1-(4-(4-methylthiazol-5-yl)phenyl)ethyl)carbamoyl)pyrrolidine-1-carbonyl)-6,6-dimethyl-4,5,6,7-tetrahydrobenzo[d]isoxazole-3-carboxamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

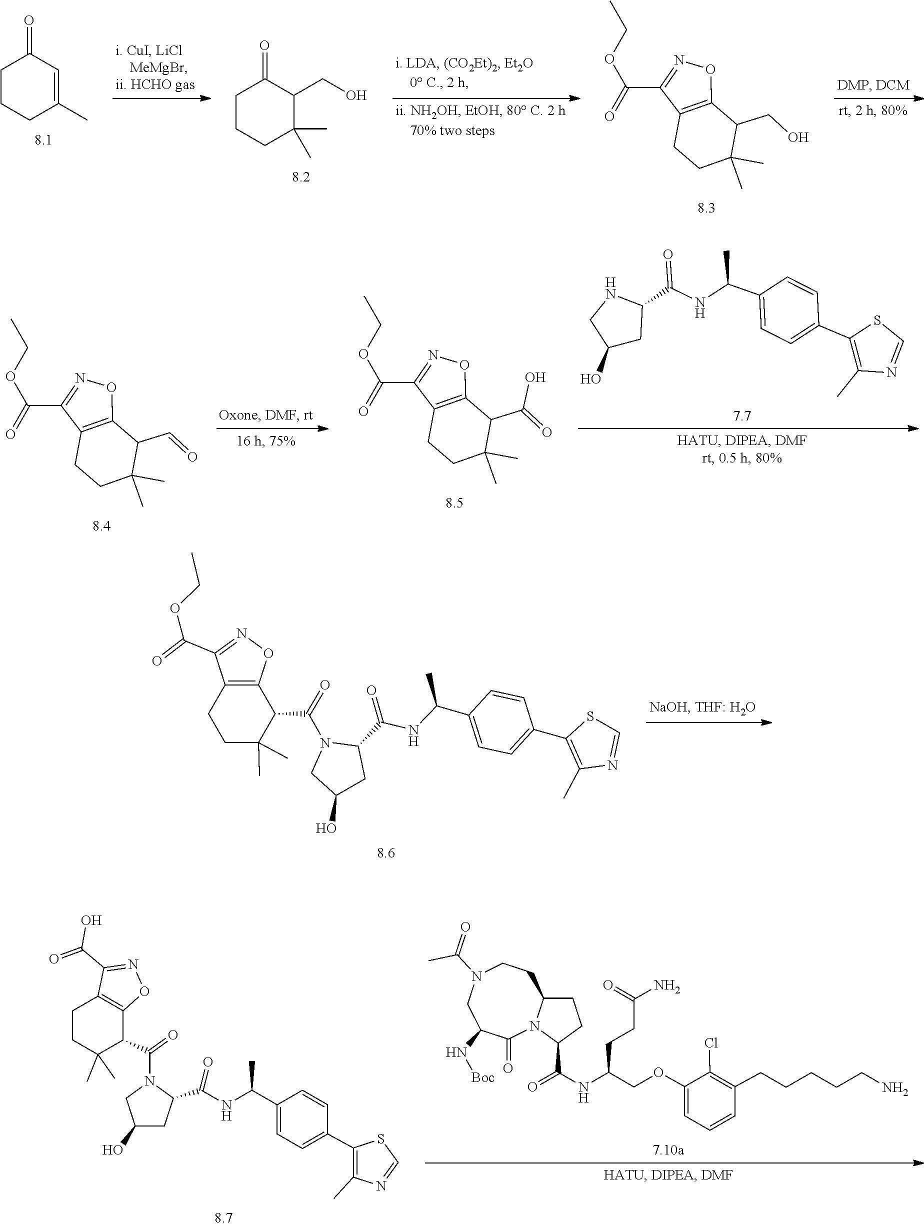

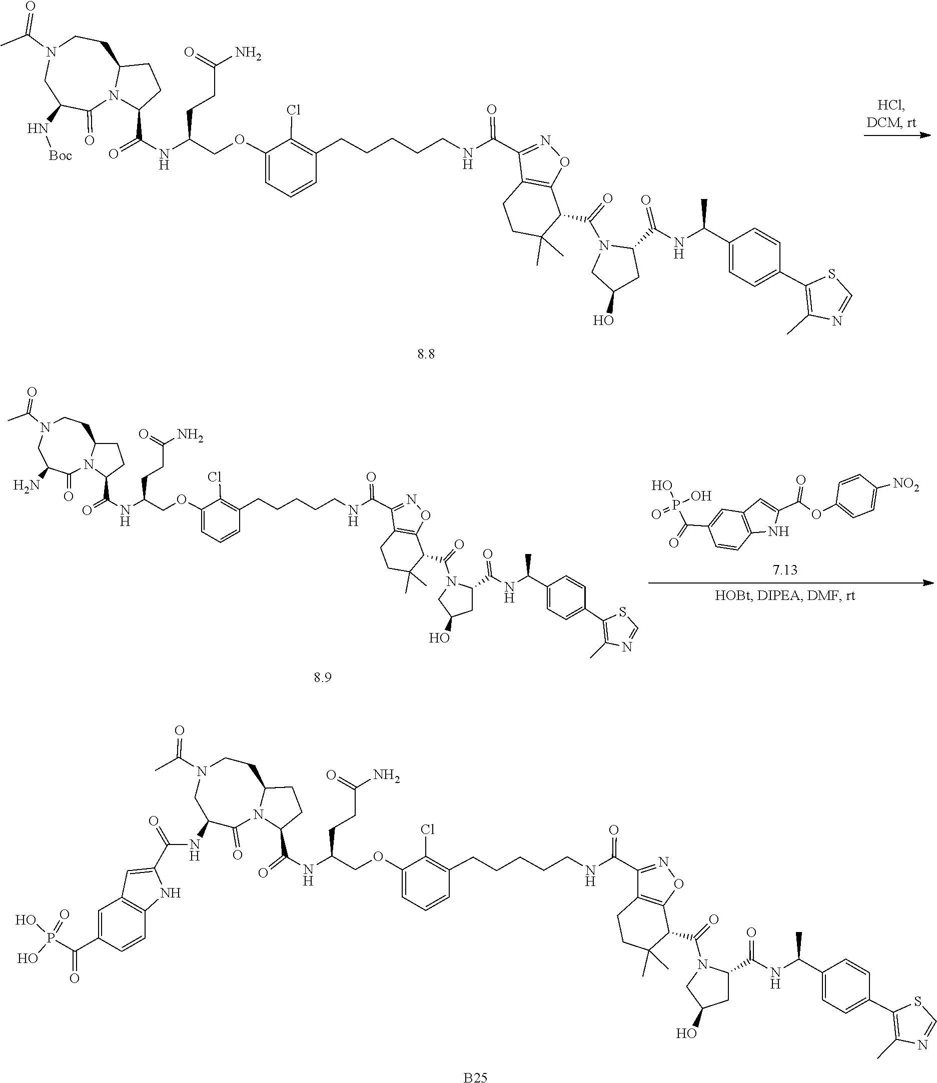

### Step 1

LiCl (0.1 eq) and CuI (0.05 eq) were added to a flame-dried two-way round bottom flask and purged with nitrogen three times. THF was added to the mixture and stirred vigorously until a homogeneous light-yellow solution. Compound 8.1 was added, and the reaction was cooled to 0° C. Methyl magnesium bromide (1.2 eq) was added to the reaction dropwise over 20 mins and the reaction was stirred at the same temperature for 2 h and monitored by TLC chromatography. After the complete conversion of starting material, the reaction was cooled to −10° C., and formaldehyde gas (generated by heating paraformaldehyde at 140° C.) was passed through the solution using a cannula for 4 h. The reaction was monitored by TLC chromatography. Upon completion of the reaction, the mixture was quenched with a saturated solution of ammonium chloride and extracted with ethyl acetate. The product was purified by flash column chromatography.

### Step 2

A solution of compound 8.2 (1 eq), diethyl oxalate (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

A solution of crude product and hydroxyl amine hydrochloride (2 eq) in ethanol was heated to reflux for 2 h. After complete conversion of starting material, ethanol was evaporated under reduced pressure and the product was purified with flash column chromatography. UPLC-MS: calculated [M+H]⁺: 254.13, found: 254.17.

### Step 3

To a stirred solution of compound 8.3 in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography. UPLC-MS: calculated [M+H]⁺: 252.12, found: 252.07.

### Step 4

To a stirred solution of compound 8.4 (1 eq) in DMF at rt, Oxone monohydrate (3 eq) was added. The reaction was stirred at rt for 16 h followed by dilution with water. The mixture was extracted with ethyl acetate and the product was purified using flash column chromatography. UPLC-MS: calculated [M+H]⁺: 268.11, found: 268.04.

### Step 5

To a stirred solution of compound 8.5 (1 eq), compound 7.7 (1 eq), and HATU (1 eq) in DMF at rt, DIPEA (1.5 eq) was added dropwise and monitored by UPLC. After completion of the reaction, the product was purified by reverse phase column chromatography. UPLC-MS: calculated [M+H]⁺: 581.24, found: 581.10.

### Step 6

To a stirred solution of compound 8.6 in THF:water (1:1) at rt, NaOH was added and the reaction was stirred for 2 h. After complete conversion of the starting material, it was acidified with 2 N HCl up to pH 3 and extracted with ethyl acetate. The product was purified with flash column chromatography. UPLC-MS: calculated [M+H]⁺: 553.20, found: 553.12.

### Step 7

To a stirred solution of compound 8.7 (1 eq), compound 7.10a (1 eq), and HATU (1 eq) in DMF at rt, DIPEA (1.5 eq) was added dropwise and monitored by UPLC. After completion of the reaction, the product was purified by preparative HPLC (ACN: H₂O with 0.1% TFA). UPLC-MS: calculated [M+2H]²*/2: 607.27, found: 607.15.

### Step 8

To a stirred solution of 8.8 in DCM, 4 N HCl in dioxane was added and stirred for 1 h at room temperature and monitored by UPLC. After completion of the reaction solvent was evaporated to dryness and the crude product was used for the next step. UPLC-MS: calculated [M+H]⁺: 1113.49, found: 1113.65.

### Step 9

To a stirred solution of compound 8.9 (1 eq), compound 7.13 (1 eq), and HOBt (1 eq) in DMF at rt, DIPEA (1.5 eq) was added dropwise and monitored by UPLC. After completion of the reaction, the product was purified by preparative HPLC to get B25 as white solid (ACN: H₂O with 0.1% TFA). UPLC-MS: calculated [M+2H]²*/2: 682.7, found: 683.08.

### Compound B23. (2-(((5S,8S,10aR)-3-acetyl-8-(((S)-5-amino-1-(2-chloro-3-(5-((S)-3-((2S,4R)-4-hydroxy-1-((R)-3,7,7-trimethyl-5,6,7,8-tetrahydro-4H-cyclohepta[d]isoxazole-8-carbonyl)pyrrolidine-2-carboxamido)-3-(4-(4-methylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

### Compound B28. (2-(((5S,8S,10aR)-3-acetyl-8-(((S)-5-amino-1-(2-chloro-3-(5-((S)-3-((2S,4R)-1-((R)-3-cyclopropyl-7,7-dimethyl-5,6,7,8-tetrahydro-4H-cyclohepta[d]isoxazole-8-carbonyl)-4-hydroxypyrrolidine-2-carboxamido)-3-(4-(4-methylthiazol-5-yl)phenyl)propanamido)pentyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

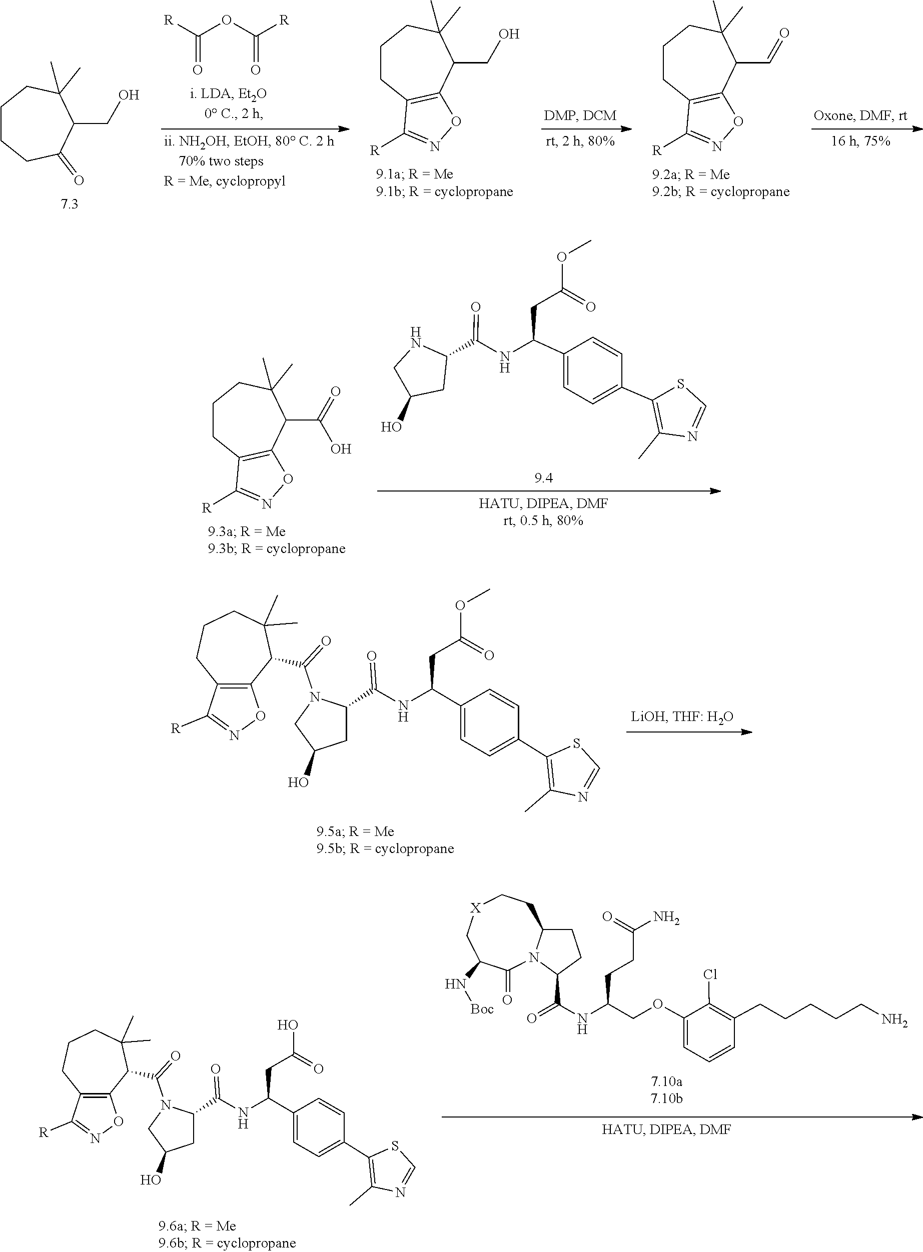

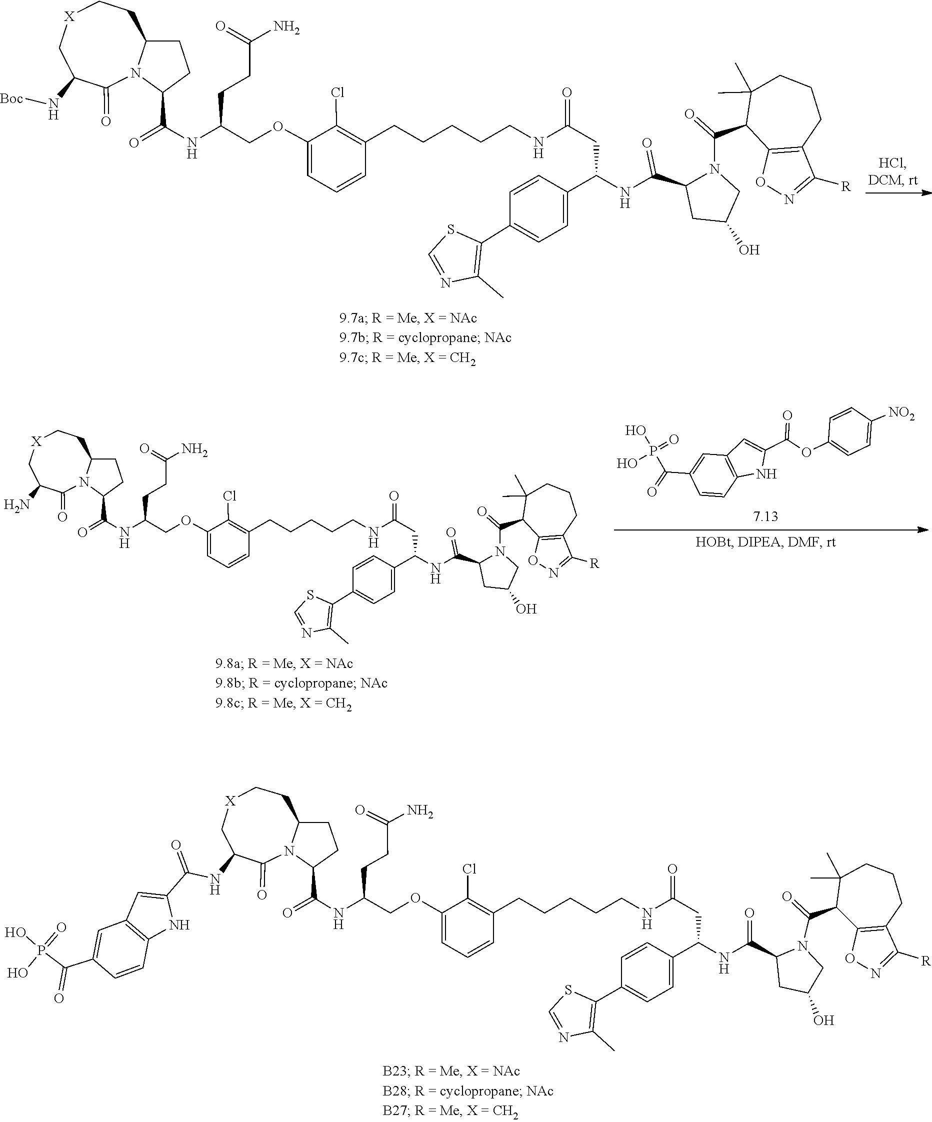

### Step 1

A solution of compound 7.3 (1 eq), acetic anhydride (1.5 eq) in diethyl ether was added dropwise to a stirred solution of LDA (2.2 eq) at 0° C. and the reaction was stirred for 2 h. After complete conversion of the starting material, the reaction was diluted with water and acidified with 2 N HCl up to pH 5. The reaction was extracted with ethyl acetate. The organic layer was separated, washed with brine, and dried over Na₂SO₄. The solvent was evaporated, and the crude product was used for the next step.

A solution of crude product and hydroxyl amine hydrochloride (2 eq) in ethanol was heated to reflux for 2 h. After complete conversion of starting material, ethanol was evaporated under reduced pressure and the product was purified with flash column chromatography to get compound 9.1a (UPLC-MS: calculated [M+H]⁺: 210.15, found: 210.17).

### Step 2

To a stirred solution of compound 9.1a in DCM, Martin's reagent was added at rt and stirred for 4 h. After completion of the reaction, it was quenched with a saturated solution Na₂S₂O₃ and extracted with DCM. The organic layer was separated, washed with brine, and dried over Na₂SO₄. The product was purified with flash column chromatography to get compound 9.2a (UPLC-MS: calculated [M+H]⁺: 208.14, found: 208.17).

### Step 3

To a stirred solution of compound 9.2a (1 eq) in DMF at rt, Oxone monohydrate (3 eq) was added. The reaction was stirred at rt for 16 h followed by dilution with water. The mixture was extracted with ethyl acetate and the product was purified using flash column chromatography. 9.3a (UPLC-MS: calculated [M+H]⁺: 224.12, found: 224.16).

### Step 4

To a stirred solution of compound 9.3a (1 eq), compound 9.4 (1 eq), and HATU (1 eq) in DMF at rt, DIPEA (1.5 eq) was added dropwise and monitored by UPLC. After completion of the reaction, the product was purified by reverse phase column chromatography to get compound 9.5a UPLC-MS: calculated [M+H]⁺: 595.25, found: 595.30.

### Step 5

To a stirred solution of compound 9.5a in THF:water (1:1) at rt, LiOH was added and the reaction was stirred for 2 h. After complete conversion of the starting material, it was acidified with 2 N HCl up to pH 3 and extracted with ethyl acetate. The product was purified with flash column chromatography to get compound 9.6a UPLC-MS: calculated [M+H]⁺: 581.24, found: 581.32.

### Step 6

To a stirred solution of compound 9.6a (1 eq), compound 7.10a (1 eq), and HATU (1 eq) in DMF at rt, DIPEA (1.5 eq) was added dropwise and monitored by UPLC. After completion of the reaction, the product was purified by preparative HPLC (ACN: H₂O with 0.1% TFA). UPLC-MS: calculated [M+2H]²+/2: 621.29, found: 621.15.

### Step 7

To a stirred solution of 9.7a in DCM, 4 N HCl in dioxane was added and stirred for 1 h at room temperature and monitored by UPLC. After completion of the reaction solvent was evaporated to dryness and the crude product was used for the next step. UPLC-MS: calculated [M+H]⁺: 1141.52, found: 1141.59.

### Step 8

To a stirred solution of compound 9.8a (1 eq), compound 7.13 (1 eq), and HOBt (1 eq) in DMF at rt, DIPEA (1.5 eq) was added dropwise and monitored by UPLC. After completion of the reaction, the product compound B23 was purified by preparative HPLC (ACN: H₂O with 0.1% TFA). UPLC-MS: calculated [M+2H]²*/2: 696.76, found: 697.03.

Similar synthetic route was followed to synthesize compound B28. UPLC-MS: calculated [M+2H]²*/2: 709.77, found: 710.05.

Similar synthetic route was followed to synthesize compound B27. UPLC-MS: calculated [M+2H]²*/2: 675.25, found: 675.50.

### Compound B24. (2-(((5S,8S,10aR)-3-acetyl-8-(((S)-5-amino-1-(2-chloro-3-(3-(1-((R)-2-((2S,4R)-1-((R)-3-cyclopropyl-7,7-dimethyl-5,6,7,8-tetrahydro-4H-cyclohepta[d]isoxazole-8-carbonyl)-4-hydroxypyrrolidine-2-carboxamido)-2-(4-(4-methylthiazol-5-yl)phenyl)ethyl)-1H-1,2,3-triazol-4-yl)propyl)phenoxy)-5-oxopentan-2-yl)carbamoyl)-6-oxodecahydropyrrolo[1,2-a][1,5]diazocin-5-yl)carbamoyl)-1H-indole-5-carbonyl)phosphonic acid

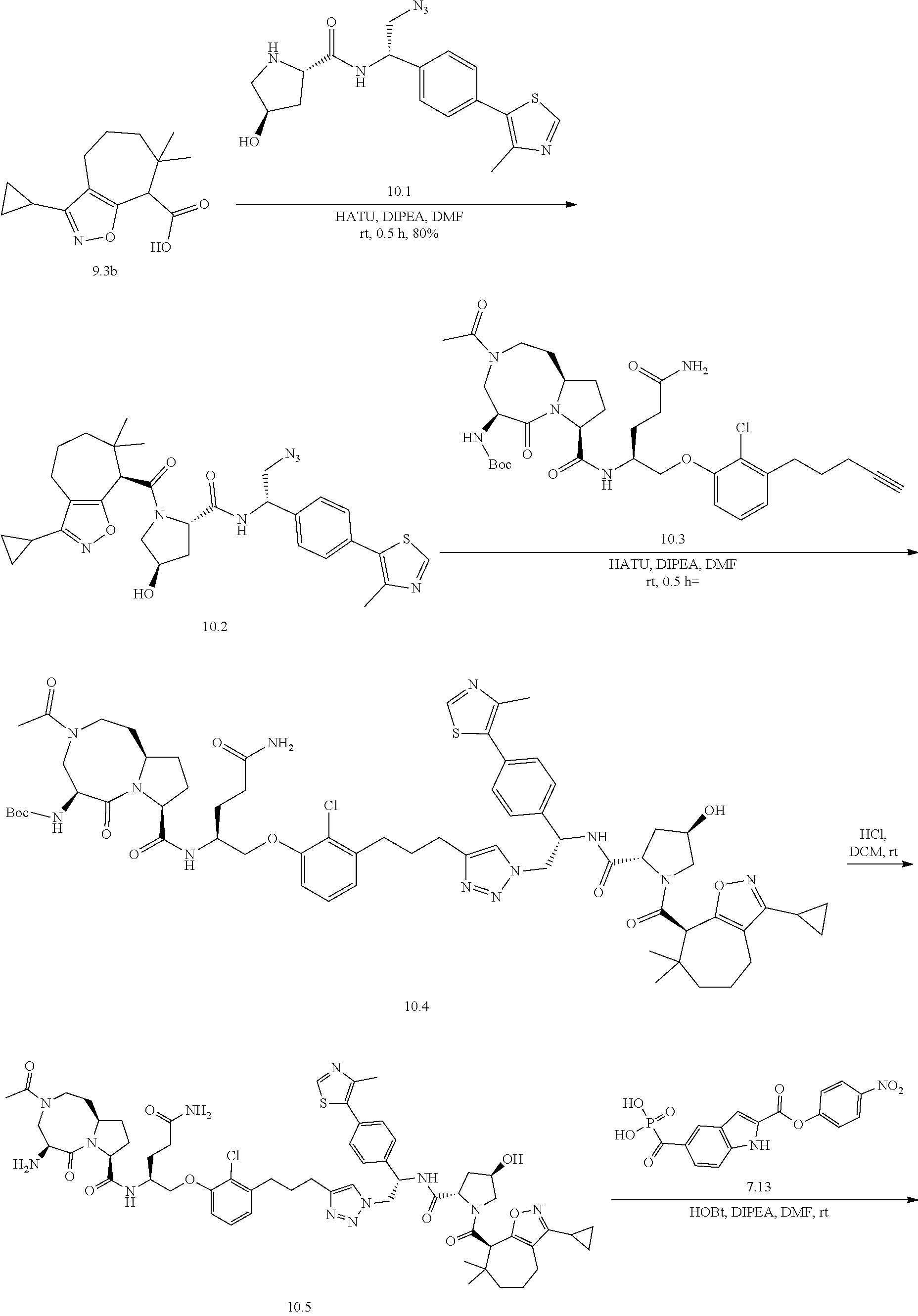

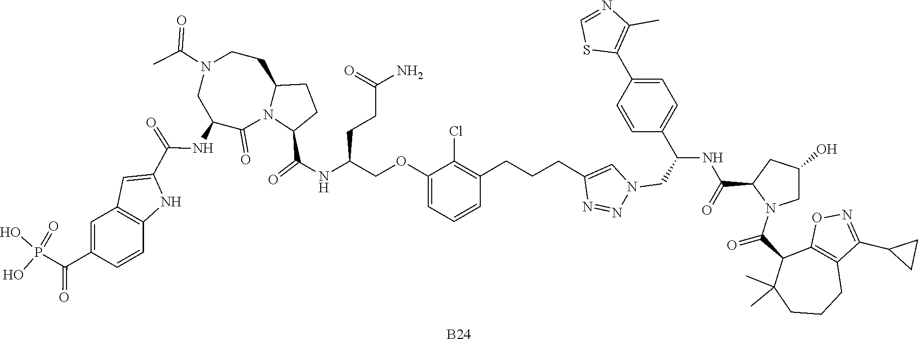 [SMILES: CC(=O)N1CC[C@H]2CC[C@@H](C(=O)N[C@@H](CCC(N)=O)COc3cccc(CCCc4cn(C[C@@H](NC(=O)[C@H]5C[C@H](O)CN5C(=O)[C@H]5c6onc(C7CC7)c6CCCC5(C)C)c5ccc(-c6scnc6C)cc5)nn4)c3Cl)N2C(=O)[C@@H](NC(=O)c2cc3cc(C(=O)P(=O)(O)O)ccc3[nH]2)C1 ; "B24"]

### Step 1

To a stirred solution of compound 9.3b (1 eq), compound 10.1 (1 eq), and HATU (1 eq) in DMF at rt, DIPEA (1.5 eq) was added dropwise and monitored by UPLC. After completion of the reaction, the product was purified by reverse phase column chromatography to get compound 10.2 UPLC-MS: calculated [M+H]H:604.26, found: 604.30.

### Step 2

To a stirred solution of compound 10.2 (1 eq), compound 10.3 (1 eq), sodium ascorbate (1.5 eq) in t-BuOH:H₂O (3:1), coper sulphate pentahydrate was added and monitored by UPLC. After completion of the reaction, the product was purified by reverse phase column chromatography to get compound 10.4 UPLC-MS: calculated [M+2H]+/2:632.75, found: 632.95.

### Step-3

To a stirred solution of 10.4 in DCM, 4 N HCl in dioxane was added and stirred for 1 h at room temperature and monitored by UPLC. After completion of the reaction solvent was evaporated to dryness to get compound 10.5. UPLC-MS: calculated [M+H]⁺: 1163.52, found: 1163.65.

### Step 4

To a stirred solution of compound 10.5 (1 eq), compound 7.13 (1 eq), and HOBt (1 eq) in DMF at rt, DIPEA (1.5 eq) was added dropwise and monitored by UPLC. After completion of the reaction, the product compound B24 was purified by preparative HPLC (ACN: H₂O with 0.1% TFA). UPLC-MS: calculated [M+2H]²*/2: 707.76, found: 708.10.

### Compound b29. (2-(((5S,8S,10aR)-2-acetyl-8-(((S)-5-amino-1-(2-chloro-3-(3-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-7,8-dihydro-4H,6H-[1,2,3]triazolo[5,1-c][1,4]oxazepin-3-yl)propyl)phenoxy)-5-oxopentan-2-yl)carbamoyl)-6-oxodecahydropyrrolo[1,2-a][1,4]diazocin-5-yl)carbamoyl)-1H-indole-5-carbonyl)phosphonic acid

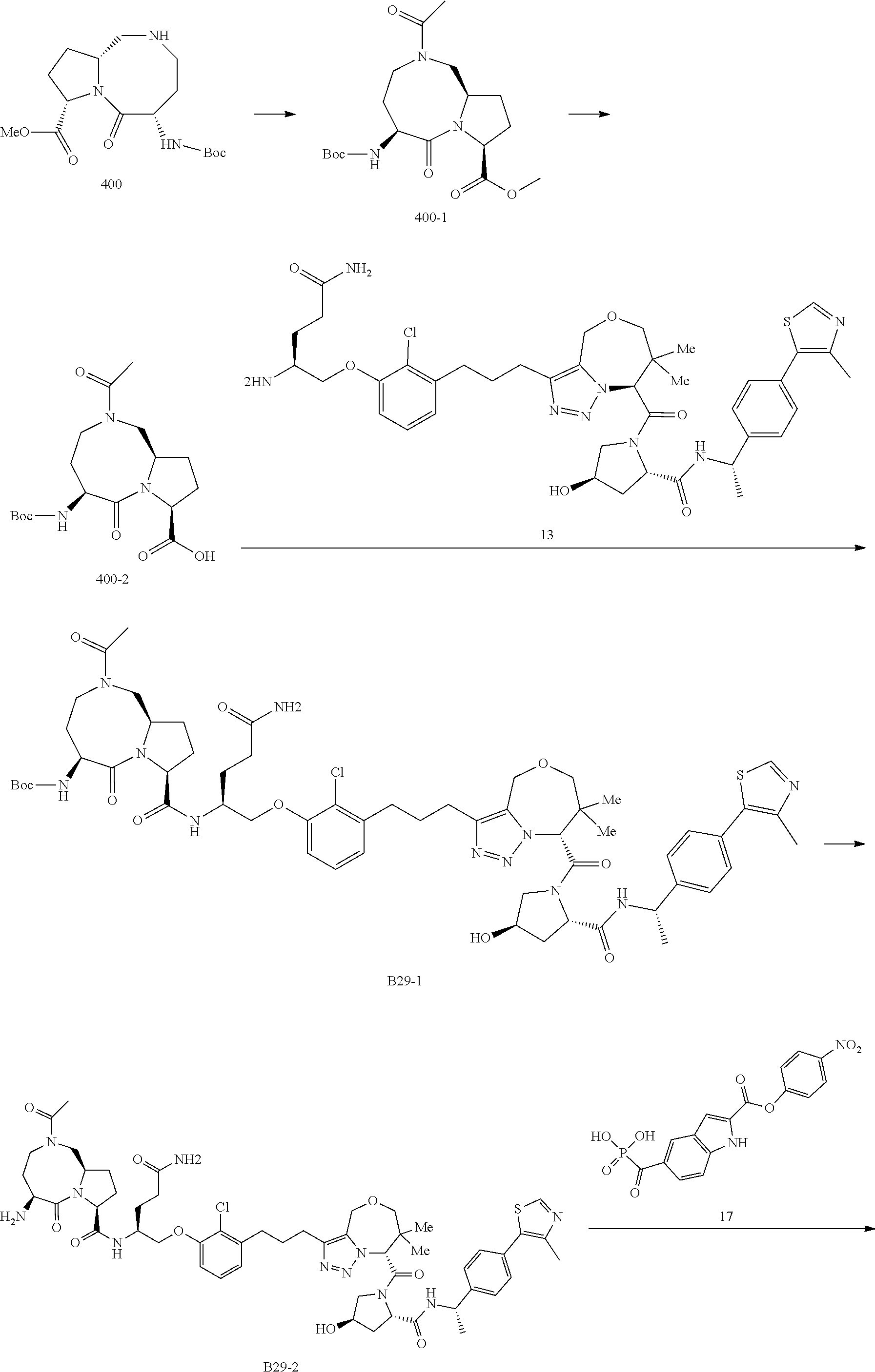

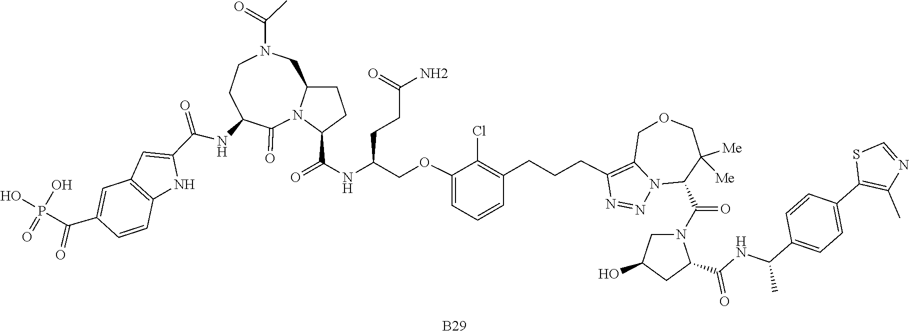 [SMILES: CC(=O)N1CC[C@H](NC(=O)c2cc3cc(C(=O)P(=O)(O)O)ccc3[nH]2)C(=O)N2[C@H](CC[C@H]2C(=O)N[C@@H](CCC(N)=O)COc2cccc(CCCc3nnn4c3COCC(C)(C)[C@@H]4C(=O)N3C[C@H](O)C[C@H]3C(=O)N[C@@H](C)c3ccc(-c4scnc4C)cc3)c2Cl)C1 ; "B29"]

Acetic anhydride (1.5 mmoL, 1.5 equiv.) and DIEA (2 mmol, 2 equiv.) were added to a solution of 400 (1 mmol, 1 equiv.) in DCM (15 mL). The resulting reaction mixture was stirred for half an hour and then was evaporated, and the residue was purified by flash chromatography on silica gel to afford 400-1. LiOH (1 mmol, 2 equiv.) was added to a mixture of 400-1 (0.5 mmol, 1 equiv.) in THF (10 mL) and Water (10 mL). The resulted mixture was stirred for 1 h, and the pH value was adjusted to 6. Ethyl Acetate was added and the orgic layer was separated and dried over Sodium sulfate. Removal of the solvent yielded 400-2. UPLC-MS calculated [M+H]+: 370.2, found: 370.5.

HATU (0.022 mmol, 1.1 equiv.) was added to a solution of 13 (0.02 mmol, 1 equiv.), 400-2 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield B29-1. UPLC-MS calculated [M+H]⁺: 1158.5, found: 1158.8.

B29-1 (20 mg) was treated with 1 mL of 1M HCl (in Methanol) and 1 mL of 4M HCl (in dioxane) for 2 h. The reaction mixture was evaporated to give the B29-2 without further purification. UPLC-MS calculated [M+H]⁺: 1058.5, found: 1058.6.

DIEA (0.042 mmol, 3 equiv.) was added to the mixture of 17 (0.017 mmol, 1.2 equiv.), compound B29-2 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B29. UPLC-MS calculated [M+2H]²⁺: 655.2, found: 655.4.

### Compound B30. (2-(((4S,7S,9aR)-2-acetyl-7-(((S)-5-amino-1-(2-chloro-3-(3-((R)-8-((2S,4R)-4-hydroxy-2-(((S)-1-(4-(4-methylthiazol-5-yl)phenyl)ethyl)carbamoyl)pyrrolidine-1-carbonyl)-7,7-dimethyl-7,8-dihydro-4H,6H-[1,2,3]triazolo[5,1-c][1,4]oxazepin-3-yl)propyl)phenoxy)-5-oxopentan-2-yl)carbamoyl)-5-oxooctahydro-1H-pyrrolo[1,2-a][1,4]diazepin-4-yl)carbamoyl)-1H-indole-5-carbonyl)phosphonic acid

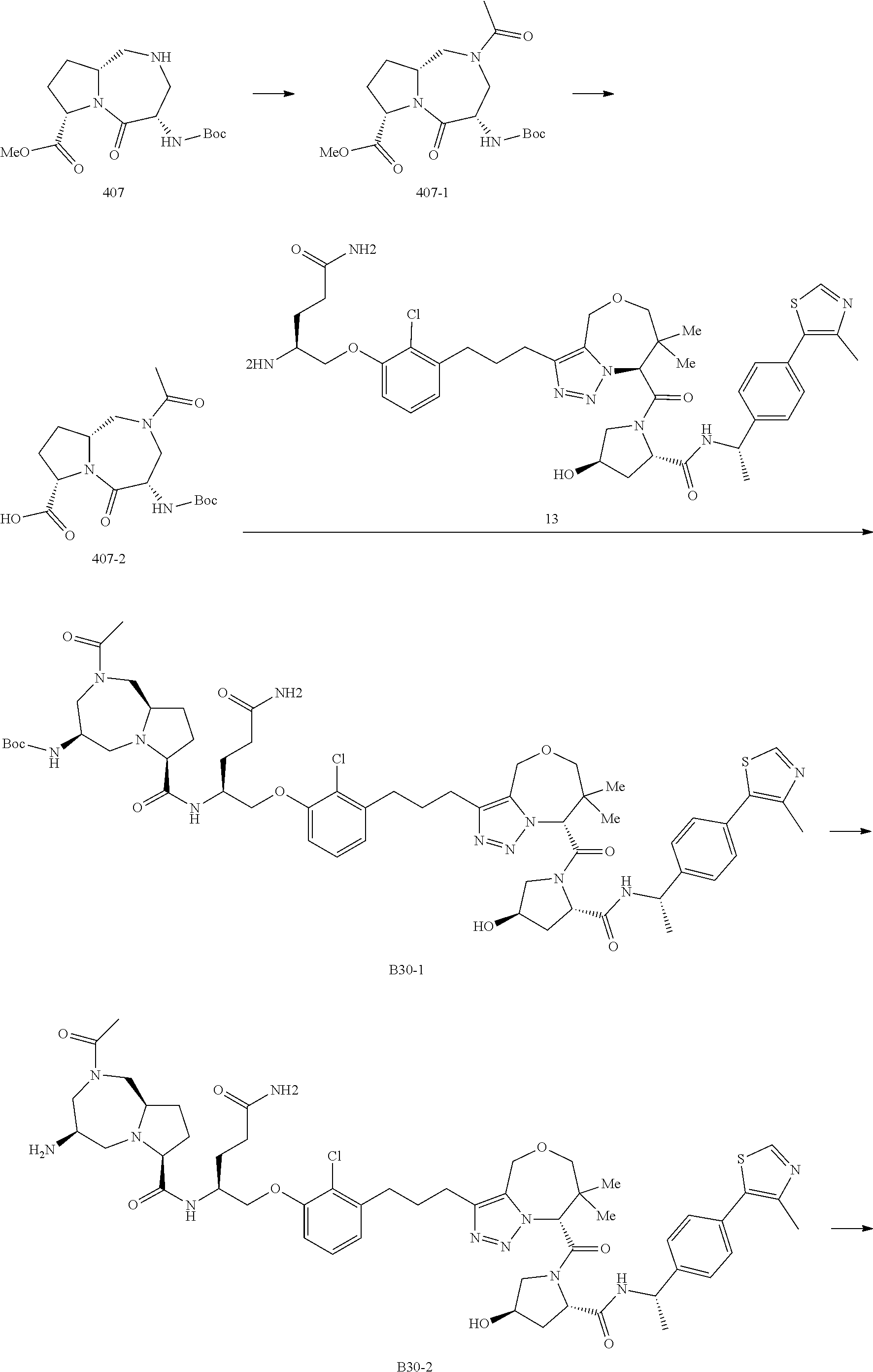

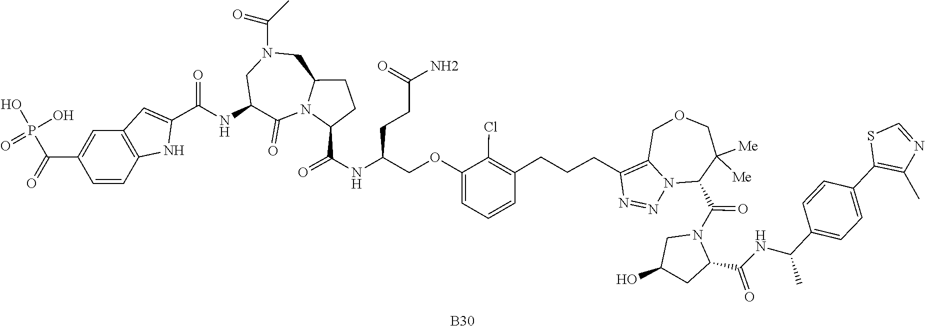

Acetic anhydride (1.5 mmoL, 1.5 equiv.) and DIEA (2 mmol, 2 equiv.) were added to a solution of 407 (1 mmol, 1 equiv.) in DCM (15 mL). The resulting reaction mixture was stirred for half an hour and then was evaporated, and the residue was purified by flash chromatography on silica gel to afford 407-1. LiOH (1 mmol, 2 equiv.) was added to a mixture of 407-1 (0.5 mmol, 1 equiv.) in THF (10 mL) and Water (10 mL). The resulted mixture was stirred for 1 h, and the pH value was adjusted to 6. Ethyl Acetate was added and the orgic layer was separated and dried over Sodium sulfate. Removal of the solvent yielded 407-2. UPLC-MS calculated [M+H]+: 356.2, found: 356.5.

HATU (0.022 mmol, 1.1 equiv.) was added to a solution of 13 (0.02 mmol, 1 equiv.), 407-4 (0.02 mmol, 1 equiv.) and DIEA (0.06 mmol, 3 equiv.) in DMF (1 mL) and the resulting mixture was stirred at rt for 1 h. This mixture was purified by reverse phase preparative HPLC to yield B30-1. UPLC-MS calculated [M+H]⁺: 1144.5, found: 1144.7.

B30-1 (20 mg) was treated with 1 mL of 1M HCl (in Methanol) and 1 mL of 4M HCl (in dioxane) for 2 h. The reaction mixture was evaporated to give the B30-2 without further purification. UPLC-MS calculated [M+H]⁺: 1054.5, found: 1044.4.

DIEA (0.042 mmol, 3 equiv.) was added to the mixture of 17 (0.017 mmol, 1.2 equiv.), compound B30-2 (0.014 mmol, 1 equiv.) and HOBt (0.028 mmol, 2 equiv.) in DMF (1 mL). The resulted mixture was stirred at room temperature for 1 hour. Purification of this reaction mixture by HPLC gave the compound B30. UPLC-MS calculated [M+2H]²⁺: 648.2, found: 648.6.

TABLE E2
Mass Spec Data

| | Expected | Observed |
|---|---|---|
| Compound No. | [M + 2H]²⁺/2 | [M + 2H]²⁺/2 |
| B1 | 710.8 | 710.9 |
| B2 | 689.3 | 689.3 |
| B3 | 655.2 | 655.4 |
| B4 | 695.8 | 695.9 |
| B5 | 731.3 | 731.8 |
| B6 | 718.3 | 718.8 |
| B7 | 730.3 | 730.4 |
| B8 | 717.8 | 717.9 |
| B9 | 737.3 | 737.8 |
| B10 | 689.5 | 689.9 |
| B11 | 689.5 | 689.9 |
| B12 | 717.3 | 717.8 |
| B13 | 725.3 | 725.7 |
| B14 | 708.8 | 708.9 |
| B15 | 709.8 | 710.0 |
| B16 | 746.3 | 746.5 |
| B17 | 708.8 | 708.9 |
| B18 | 703.8 | 703.8 |
| B19 | 663.2 | 663.3 |
| B20 | 697.8 | 697.6 |
| B21 | 689.7 | 689.98 |
| B22 | 709.25 | 709.45 |
| B23 | 696.75 | 697.03 |
| B24 | 707.75 | 708.10 |
| B25 | 682.75 | 683.08 |
| B26 | 668.25 | 668.50 |
| B27 | 675.25 | 675.45 |
| B28 | 709.75 | 710.05 |
| B29 | 655.2 | 655.4 |
| B30 | 648.2 | 648.6 |

### II. Biological Assessment

### A. VHL Ligands

### In Vitro Assay:

### Fluorescence Polarization (FP) Assay

FP experiments were performed in 96-well, black round-bottom plates (Microfluor 2, Fisher Scientific) using the CLARIOstar microplate reader (BMG Labtech). Serial dilutions of test compounds, fluorescein-labeled tracer, and VBC protein were added to each well. The final volume in the assay buffer (PBS pH 7.4+0.01% BGG+0.01% Tween-20, 2 mM DTT) is 100 μL. The final concentration of fluorescein-labeled tracer and VBC protein were 5 nM. The plate was mixed on a shaker for 15 min and incubated at rt for 1 h to reach equilibrium. The polarization values in millipolarization (mP) units were measured at an excitation wavelength of 485 nm and an emission wavelength of 530 nm. All experimental data were analyzed using Prism 8.0 software (GraphPad Software). IC₅₀ values were determined by nonlinear regression fitting of the competition curves. Kᵢ values of competitive inhibitors were obtained directly by nonlinear regression fitting, based upon the KD values of the fluorescein-labeled tracer and concentrations of the protein and probe in the competitive assays. All the FP competitive experiments were performed in duplicate in three independent experiments. Results are summarized in Table E3.

TABLE E3

| | VHL Binding FP |
|---|---|
| Compound No. | (IC50, nM) |
| A1 | 354.3 |
| A2 | 7.0 |
| A3 | 11.6 |
| A4 | 7.5 |
| A5 | 1252 |
| A6 | 14.89 |
| A7 | 8.4 |
| A8 | 8.4 |
| A9 | 18.6 |
| A10 | 9.5 |
| A11 | |
| A12 | 2.3 |
| A13 | 33 |
| A14 | 58 |
| A15 | 5.3 |
| A16 | 8.6 |
| A17 | 107.2 |
| A18 | |
| A19 | |
| A20 | 71.5 |
| A21 | 2.84 |
| A22 | 35 |
| A23 | 15.5 |
| A24 | — |
| A25 | — |
| A26 | 11.1 |

### B. Bifunctional Degraders

### Cell Viability Assay

SU-DHL-1 and SUP-M2 cell viability was determined by CellTiter-Glo 2.0 Cell Viability Assay (Promega). SU-DHL-1 cell line was purchased from ATCC (Manassas, VA) and SUP-M2 (ACC-509) was purchased from DSMZ (Germany). RPMI 1640 medium and fetal bovine serum (FBS) were purchased from Gibco/Thermo Fisher Scientific. In a typical procedure, cells seeded at 1000 cells per well in 384-well white plates (Corning) were incubated with serially diluted compounds for 4 days at 37° C. with 5% CO2. At the end of treatment, CellTiter-Glo 2.0 was added to the wells and luminescence was acquired on TECAN SPARK plate reader. Untreated cells were used as control. Data points were fit with a four-parameter equation to generate a concentration-response curve. IC₅₀ values were calculated using a nonlinear regression analysis of the mean±SD from triplicate.

### HiBiT Assay

STAT3-HiBiT KI HeLa cell line was purchased from Promega. This cell line is a clone created by using CRISPR-Cas9 to fuse HiBiT to the 3′ end of STAT3 in HeLa cells. DMEM medium and FBS were purchased from Gibco/Thermo Fisher Scientific. STAT3 degradation was determined based on quantification of luminescent signal using Nano-Glo HiBiT Lytic Detection System. In a typical procedure, cells seeded at 20000 cells per well in 96-well white plates (Corning) were incubated with serially diluted compounds for 24 hours at 37° C. with 5% CO₂. At the end of treatment, Nano-Glo HiBiT Lytic Detection reagents was added to the wells and luminescence was acquired on TECAN SPARK plate reader. Untreated cells were used as control. Data points were fit with a four-parameter equation to generate a concentration-response curve. IC₅₀ values were calculated using a nonlinear regression analysis of the mean±SD from triplicate.

TABLE E4

| | HiBit Degradation | | Cell viability | |
|---|---|---|---|---|
| | DC50 | Dmax (%) @ | (IC50, nM), 4 d | |
| Compound No. | (nM) | 1000 nM | SU-DHL1 | SUP-M2 |
| B1 | <1 | >95 | 5 | 5 |
| B2 | 1 | >95 | | 2 |
| B3 | 6 | >95 | | 7 |
| B4 | 7 | >95 | 43 | 52 |
| B5 | 1 | >95 | 12 | 14 |
| B6 | 4 | >95 | 15 | 30 |
| B7 | 2.3 | 90 | 4 | 4 |
| B8 | 1.4 | 90 | 5 | 4 |
| B9 | 2.1 | 90 | 4 | 3 |
| B10 | >500 | <10 | >500 | >500 |
| B11 | 3.5 | 90 | 6 | 3 |
| B12 | | | | |
| B13 | | | | |
| B14 | 1.4 | | 5 | 5 |
| B15 | 5.0 | | 7.6 | 8.8 |
| B16 | 86 | | 39 | 55 |
| B17 | 5 | | 2 | 6 |
| B18 | 4 | | 3 | 12 |
| B19 | 15.3 | | 13.3 | 8.9 |
| B20 | 17.8 | | 21.0 | 25.9 |
| B21 | 3.5 | | 6 | 3 |
| B22 | 6 | | 9 | 17 |
| B23 | 3 | | 6 | 10 |
| B24 | 5 | | 3 | 9 |
| B25 | 21 | | 39 | 50 |
| B26 | 0.7 | | 0.9 | — |
| B27 | 5 | | 1.8 | — |
| B28 | 1 | | 1 | 1 |
| B29 | 6.2 | | 7.9 | 3.4 |
| B30 | 1.7 | | 5.1 | 7.6 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A conjugate of Formula II: 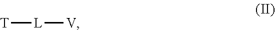
or a pharmaceutically acceptable salt, solvate, or stereoisomer thereof, wherein:
V is of Formula II-1 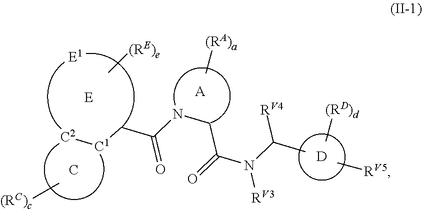
wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C¹ and C² are independently C or N;
each R^{C} is independently 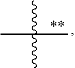
hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently 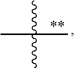
halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1″}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d};
wherein the alkyl, carbocyclyl, or heterocyclyl is optionally substituted with one or more R^{u};
each R^{E} is independently 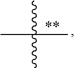
oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is 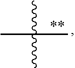
hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein one of R^{C}, R^{D}, R^{E} and R^{V4} is 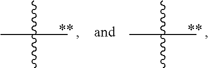
and denotes attachment to -L-T,
L is of Formula II-2 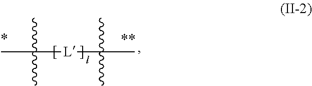
wherein:
* denotes attachment to T and ** denotes attachment to V;
each L′ is independently C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L′})—, —C(═O)O—, —N(R^{L′})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L′} is independently hydrogen, C₁₋₆ alkyl, C₂₋₆ alkenyl, C₂₋₆ alkynyl, C₃-12 carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
l is an integer selected from 0 to 6,
T is of Formula II-3 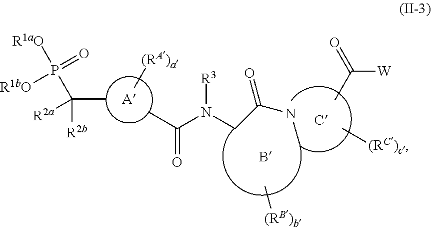
wherein:
R^{1a} and R^{1b} are independently hydrogen or C₁₋₆ alkyl optionally substituted with one or more R^{u};
R^{2a} and R^{2b} are independently hydrogen or halogen;
R^{2a} and R^{2b} together form an oxo; or
R^{2a} and R^{2b}, together with the carbon atom to which they are attached, form C₃₋₆ carbocycle or 3- to 6-membered heterocycle, wherein the carbocycle or heterocycle is optionally substituted with one or more R^{u};
Ring A′ is C₆₋₁₀ aryl or 5- to 10-membered heteroaryl;
each R^{A′} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
a′ is an integer selected from 0 to 6, as valency permits;
R³ is hydrogen, C₁-6 alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
Ring B′ is 3- to 12-membered heterocycle;
each R^{B′} is independently 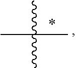
oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
* denotes attachment to L;
b′ is an integer selected from 0 to 6, as valency permits;
Ring C′ is 3- to 8-membered heterocycle;
each R^{C′} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c′ is an integer selected from 0 to 6, as valency permits;
W is 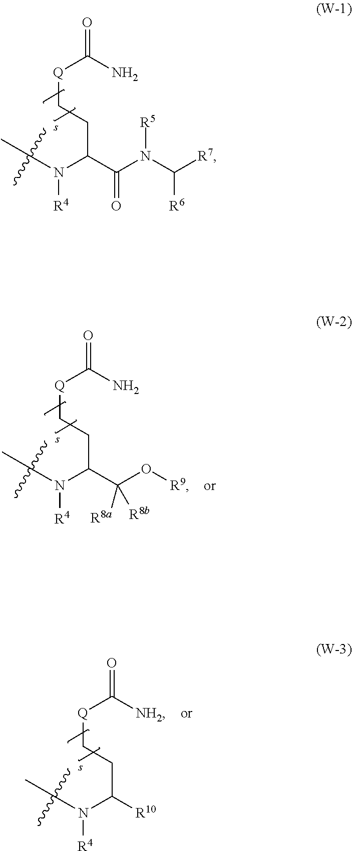
W is 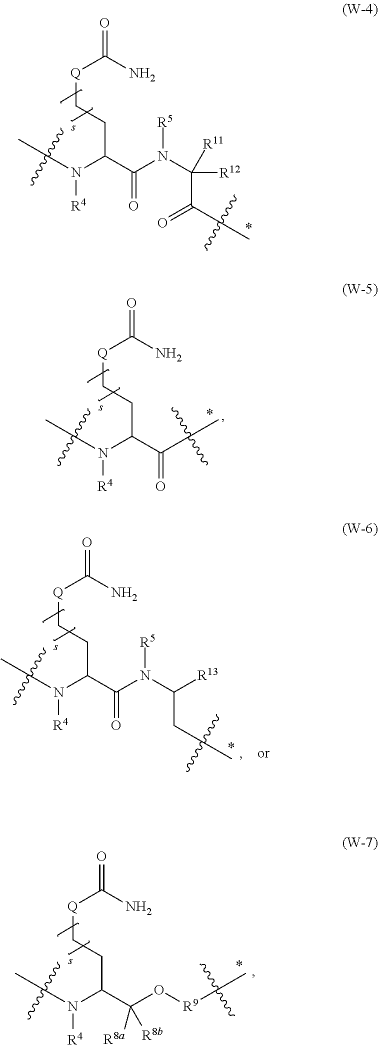
wherein:
* denotes attachment to L;
Q is absent; or
Q is —C(R^{Q})₂—, —O—, and —N(R^{Q′})—;
each R^{Q} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{Q}, together with the carbon atom to which they are attached, form C₃₋₆ carbocyclyl or 3- to 6-membered heterocyclyl, wherein the carbocyclyl or heterocyclyl is optionally substituted with one or more R^{u};
R^{Q′} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl or heterocyclyl is optionally substituted with one or more R^{u};
s is an integer selected from 0 to 4;
R⁴ and R⁵ are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
R⁶ and R⁷ are independently C₆₋₁₀ aryl or 5- to 10-membered heteroaryl, wherein the aryl or heteroaryl is optionally substituted with one or more R^{6a} or R^{7a}, respectively;
each R^{6a} and each R^{7a} are independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
or
R⁷ is hydrogen or C₁₋₆ alkyl optionally substituted with one or more R^{7b};
each R^{7b} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
or
R⁶ and R⁷, together with the carbon atom to which they are attached, form C₃₋₁₂ carbocyclyl, 3- to 12-membered heterocyclyl, C₆₋₁₀ aryl, or 5- to 10-membered heteroaryl, wherein the carbocyclyl, heterocyclyl, aryl, or heteroaryl is optionally substituted with one or more R^{u};
R^{8a} and R^{8b} are independently hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, carbocyclyl, or heterocyclyl is optionally substituted with one or more R^{u};
R⁹ is C₆₋₁₀ aryl or 5- to 10-membered heteroaryl, wherein the aryl or heteroaryl is optionally substituted with one or more R^{9a};
each R^{9a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹⁰ is hydrogen, halogen, —CN, —NO₂, —OH, —NH₂, C₁₋₆ alkyl, C₁₋₆ alkoxy, C₂₋₆ alkenyl, C₂₋₆ alkynyl, C₁₋₆ alkylamino, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
R¹¹ is hydrogen or C₁₋₆ alkyl optionally substituted with one or more R^{u};
R¹² is hydrogen, C₁₋₆ alkyl, C₃₋₁₂ carbocyclyl, 3- to 12-membered heterocyclyl, C₆₋₁₀ aryl, 5- to 10-membered heteroaryl, —(C₁₋₆ alkylene)-(C₃₋₁₂ carbocyclyl), —(C₁₋₆ alkylene)-(3- to 12-membered heterocyclyl), —(C₁₋₆ alkylene)-(C₆₋₁₀ aryl), or —(C₁₋₆ alkylene)-(5- to 10-membered heteroaryl), wherein the alkyl, alkylene, carbocyclyl, heterocyclyl, aryl, or heteroaryl is optionally substituted with one or more R^{12a};
each R^{12a} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹¹ and R¹², together with the carbon atom to which they are attached, form C₃₋₆ carbocyclyl or 3- to 6-membered heterocyclyl, wherein carbocyclyl or heterocyclyl is optionally substituted with one or more R^{u}; or
R¹¹ and R⁵, together with the intervening atoms, form 4- to 8-membered heterocyclyl optionally substituted with one or more R^{u}; and
R¹³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
wherein:
each R^{u} is independently oxo, halogen, —CN, —NO₂, —OH, —NH₂, C₁₋₆ alkyl, C₁₋₆ alkoxy, C₁-6 alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

2-112. (canceled)

113. The compound of claim 1, or a pharmaceutically acceptable salt thereof, wherein:
V is of Formula II-1-ix, II-1-x, II-1-xi, II-1-xii, or II-1-xiii: 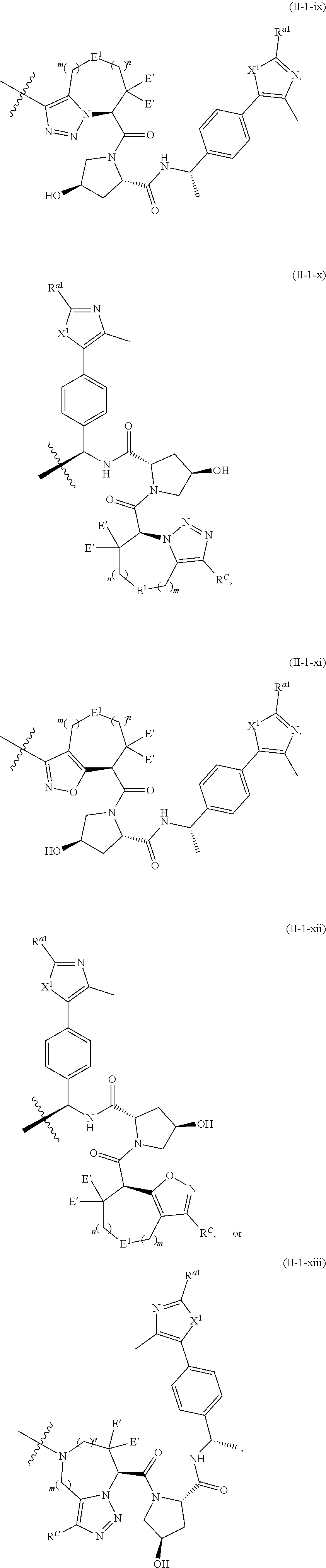
wherein:
R^{a1} is C₁₋₆ alkyl;
X¹ is O or S;
E¹ is CH₂, NR^{E1″} or O;
R^{E1″} is hydrogen, C₁₋₆ alkyl, or —C(═O)R^{a};
E′ is hydrogen or C₁₋₆ alkyl;
R^{a} is C₁₋₆ alkyl;
R^{C} is C₁₋₆ alkyl or C₃₋₆ carbocyclyl;
m is 1 or 2;
n is 0 or 1;
and 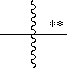
denotes attachment to -L-T;
L is of Formula II-2: 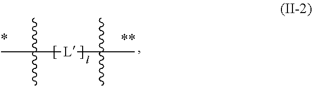
wherein:
* denotes attachment to T and ** denotes attachment to V;
each L′ is independently C₁₋₆ alkylene, 5- to 10-membered heteroarylene, —C(═O)—, —C(═O)N(R^{L′})—, —C(═O)O—, or —N(R^{L′})—;
R^{L′} is hydrogen;
l is an integer selected from 1 to 4;
T is of Formula II-3-ii-g or II-3-ii-h: 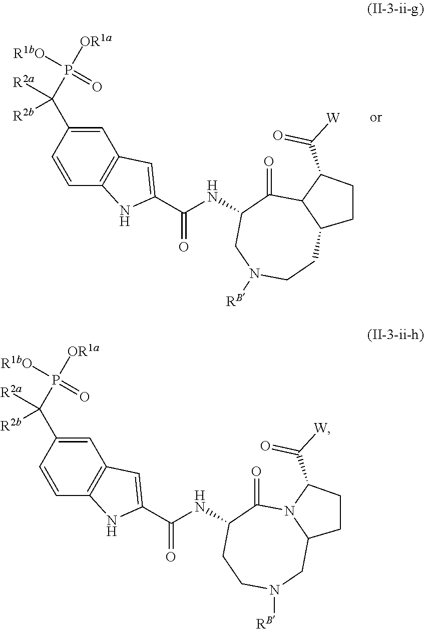
wherein:
R^{1a} and R^{1b} are independently hydrogen or C₁₋₆ alkyl;
R^{2a} and R^{2b} are independently hydrogen or halogen;
R^{2a} and R^{2b} together form an oxo; or
R^{B′} is —C(═O)R^{a} or 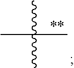
W is W-7 or W-8: 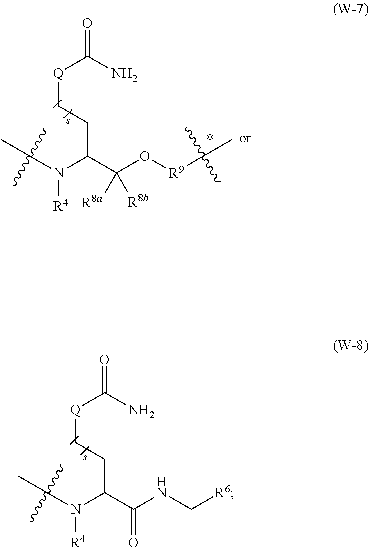
wherein:
* denotes attachment to L;
Q is absent;
s is 1;
R⁴ is hydrogen;
R⁶ is C₆₋₁₀ aryl, wherein the aryl is optionally substituted with one or more R^{6a};
each R^{6a} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d};
R^{a} is C₁₋₆ alkyl, C₃₋₁₂ carbocyclyl, 3- to 12-membered heterocyclyl, C₆₋₁₀ aryl, or 5- to 10-membered heteroaryl;
R^{b} is hydrogen, C₁₋₆ alkyl, C₃₋₁₂ carbocyclyl, 3- to 12-membered heterocyclyl, C₆₋₁₀ aryl, or 5- to 10-membered heteroaryl;
R^{c} and R^{d} are independently hydrogen, C₁₋₆ alkyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl;
R^{8a} and R^{8b} are both hydrogen;
R⁹ is C₆₋₁₀ aryl or 5- to 10-membered heteroaryl, wherein the aryl or heteroaryl is optionally substituted with one or more R^{9a}; and
each R^{9a} is independently halogen, —CN, —NO₂, —OH, —NH₂, C₁₋₆ alkyl, C₁₋₆ alkoxy, C₃₋₁₂ carbocyclyl, 3- to 12-membered heterocyclyl, C₆₋₁₀ aryl, or 5- to 10-membered heteroaryl;
with the provisos that:
(i) when R^{B′} is —C(═O)R^{a}, then W is W-7; or
(ii) when R^{B′} is 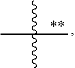
then W is W-8.

114. The compound of claim 113, or a pharmaceutically acceptable salt thereof, wherein V is of Formula II-1-xiv or II-1-xv:

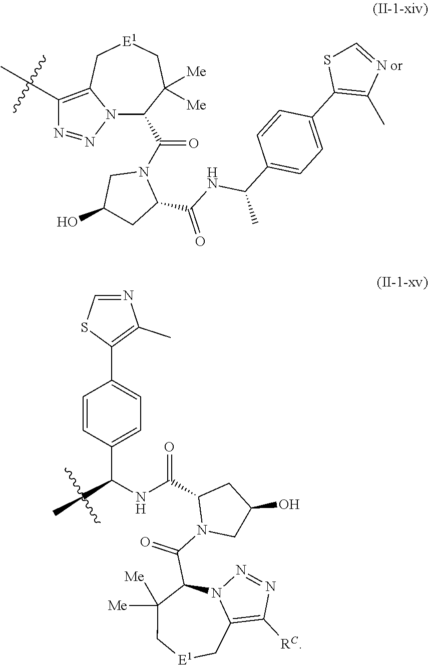

115. The compound of claim 114, or a pharmaceutically acceptable salt thereof, wherein E¹ is O.

116. The compound of claim 114, or a pharmaceutically acceptable salt thereof, wherein R^{C} is methyl or cyclopropyl.

117. The compound of claim 113, or a pharmaceutically acceptable salt thereof, wherein each L′ is independently C₁₋₆ alkylene, —C(═O)—, or —N(R^{L′})—.

118. The compound of claim 113, or a pharmaceutically acceptable salt thereof, wherein T is of Formula II-3-ii-g.

119. The compound of claim 117, or a pharmaceutically acceptable salt thereof, wherein R^{B′} is —C(═O)CH₃.

120. The compound of claim 113, or a pharmaceutically acceptable salt thereof, wherein T is of Formula II-3-ii-h.

121. The compound of claim 119, or a pharmaceutically acceptable salt thereof, wherein R^{B′} is —C(═O)CH₃.

122. The conjugate of claim 113, wherein R⁹ is:

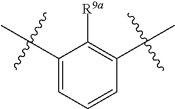

123. The conjugate of claim 122, or a pharmaceutically acceptable salt thereof, wherein R^{9a} is halogen.

124. The conjugate of claim 113, or a pharmaceutically acceptable salt thereof, wherein the conjugate is of Formula II-XI: 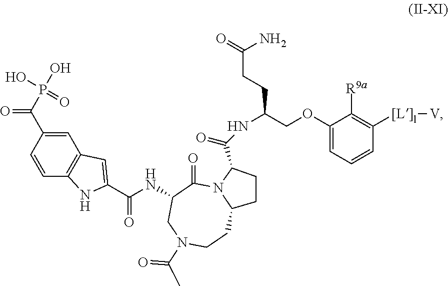
wherein:
R^{9a} is chloro;
each L′ is independently C₁₋₆ alkylene, —C(═O)—, or —N(R^{L′})—;
R^{L′} is hydrogen; and
l is an integer from 1 to 4.

125. The conjugate of claim 124, or a pharmaceutically acceptable salt thereof, wherein V is:

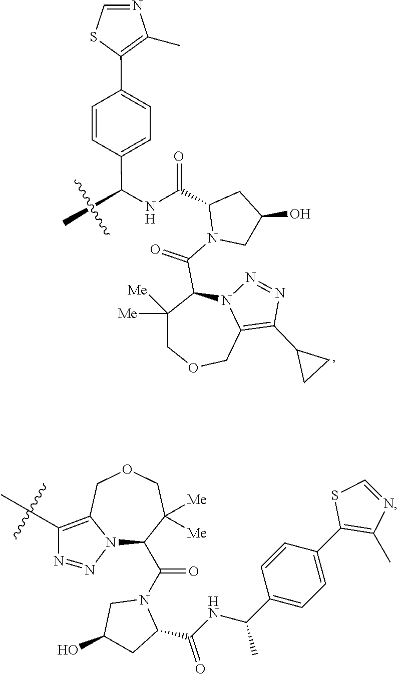

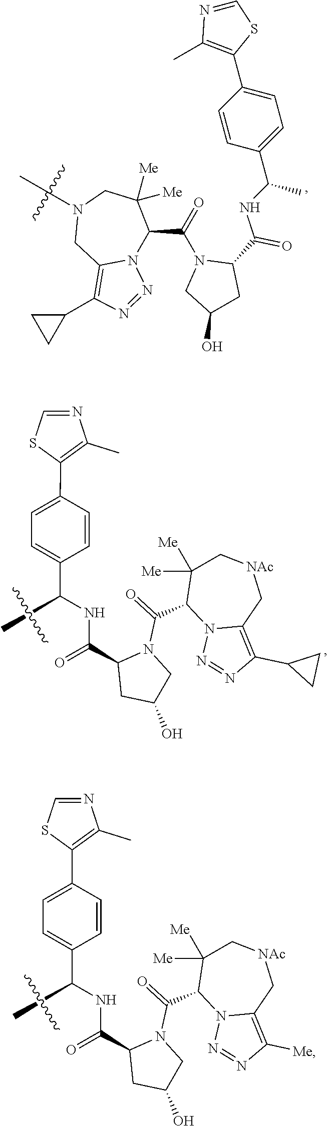

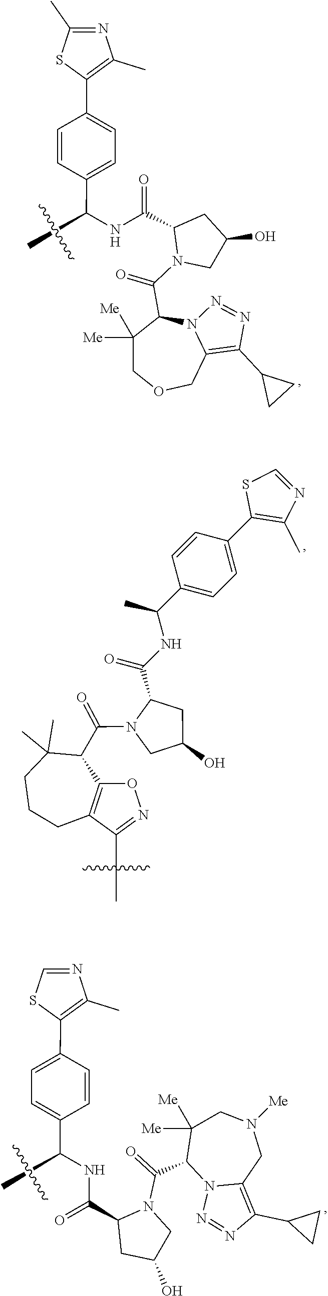

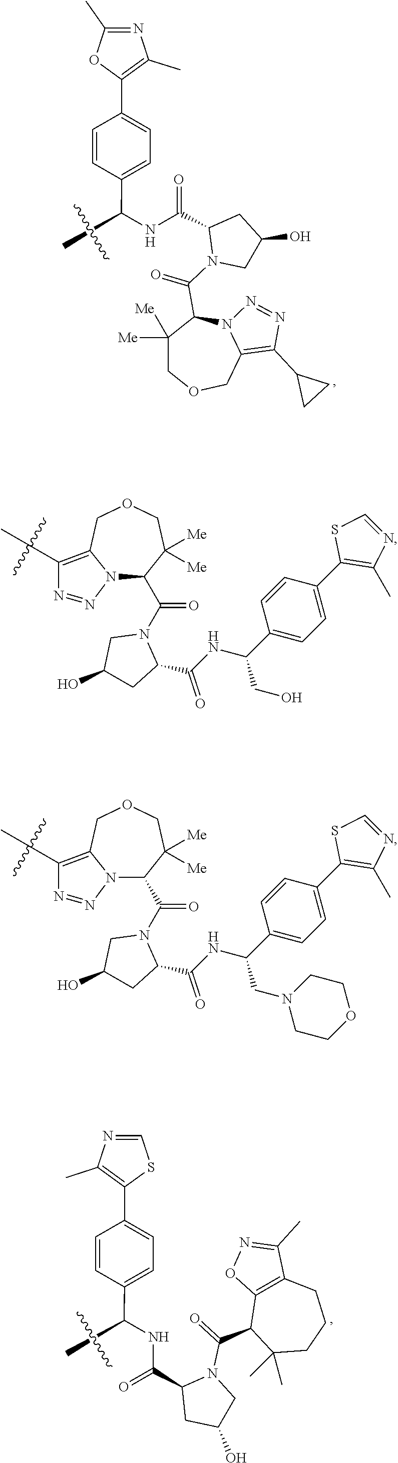

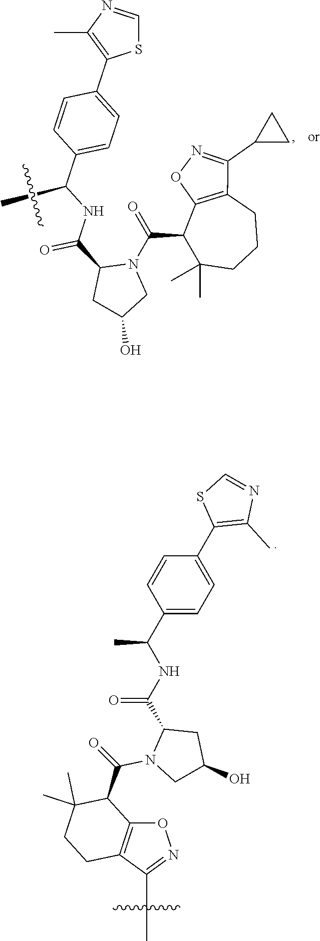

126. The conjugate of claim 113, or a pharmaceutically acceptable salt thereof, wherein the conjugate is of Formula II-XII: 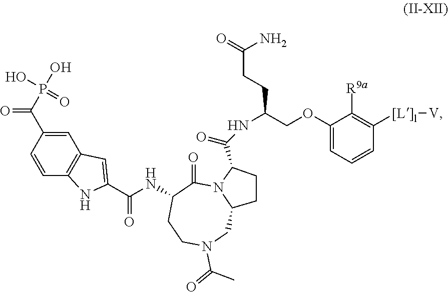
wherein:
R^{9a} is chloro;
each L′ is independently C₁₋₆ alkylene, —C(═O)—, or —N(R^{L′})—;
R^{L′} is hydrogen; and
l is an integer from 1 to 4.

127. The conjugate of claim 126, or a pharmaceutically acceptable salt thereof, wherein V is:

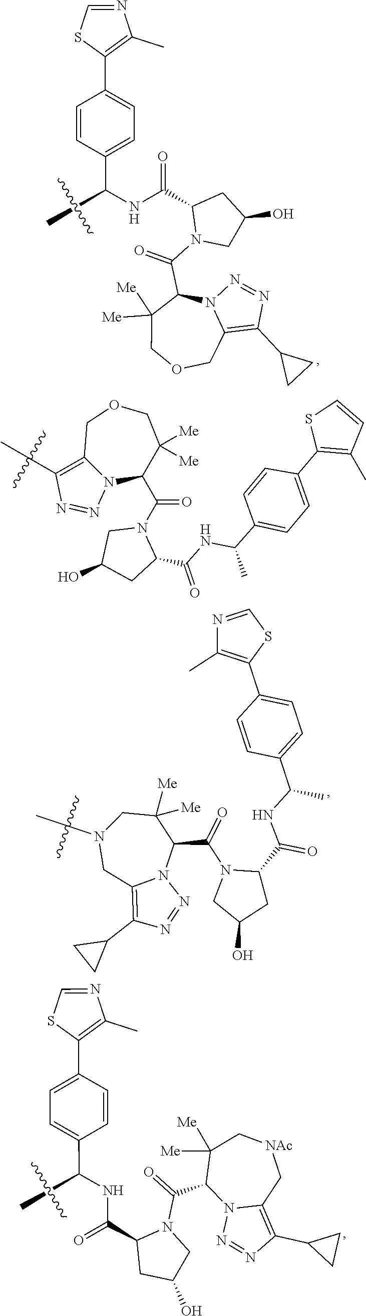

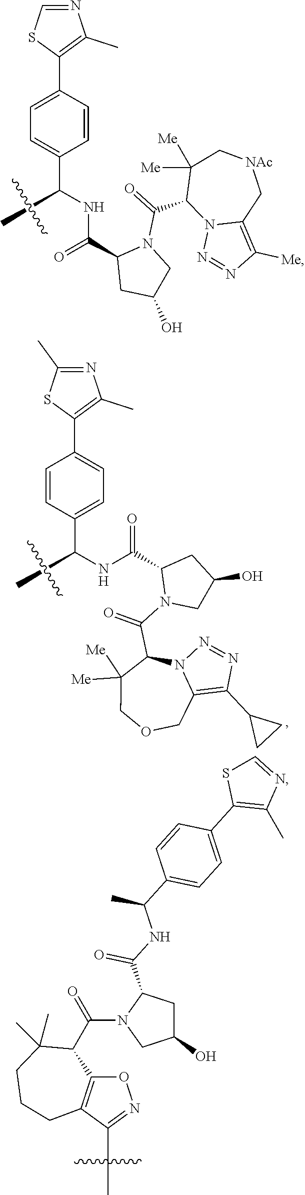

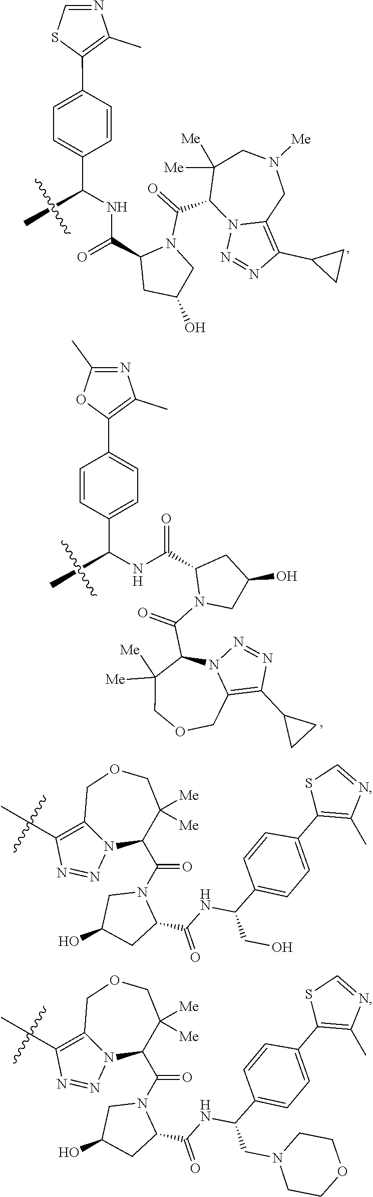

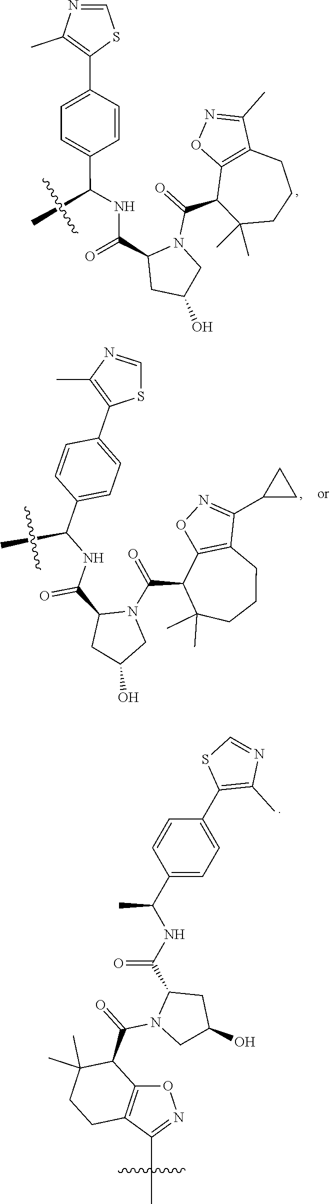

128. The conjugate of claim 113, or a pharmaceutically acceptable salt thereof, wherein the conjugate is of Formula II-XV: 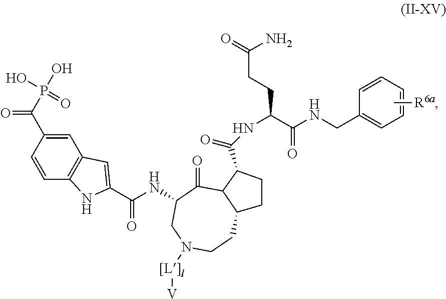
wherein:
R^{6a} is —S(═O)₂R^{a};
R^{a} is C₁₋₆ alkyl;
each L′ is independently C₁₋₆ alkylene, —C(═O)—, or —N(R^{L′})—;
R^{L′} is hydrogen; and
l is an integer from 1 to 4.

129. A conjugate selected from the group consisting of: 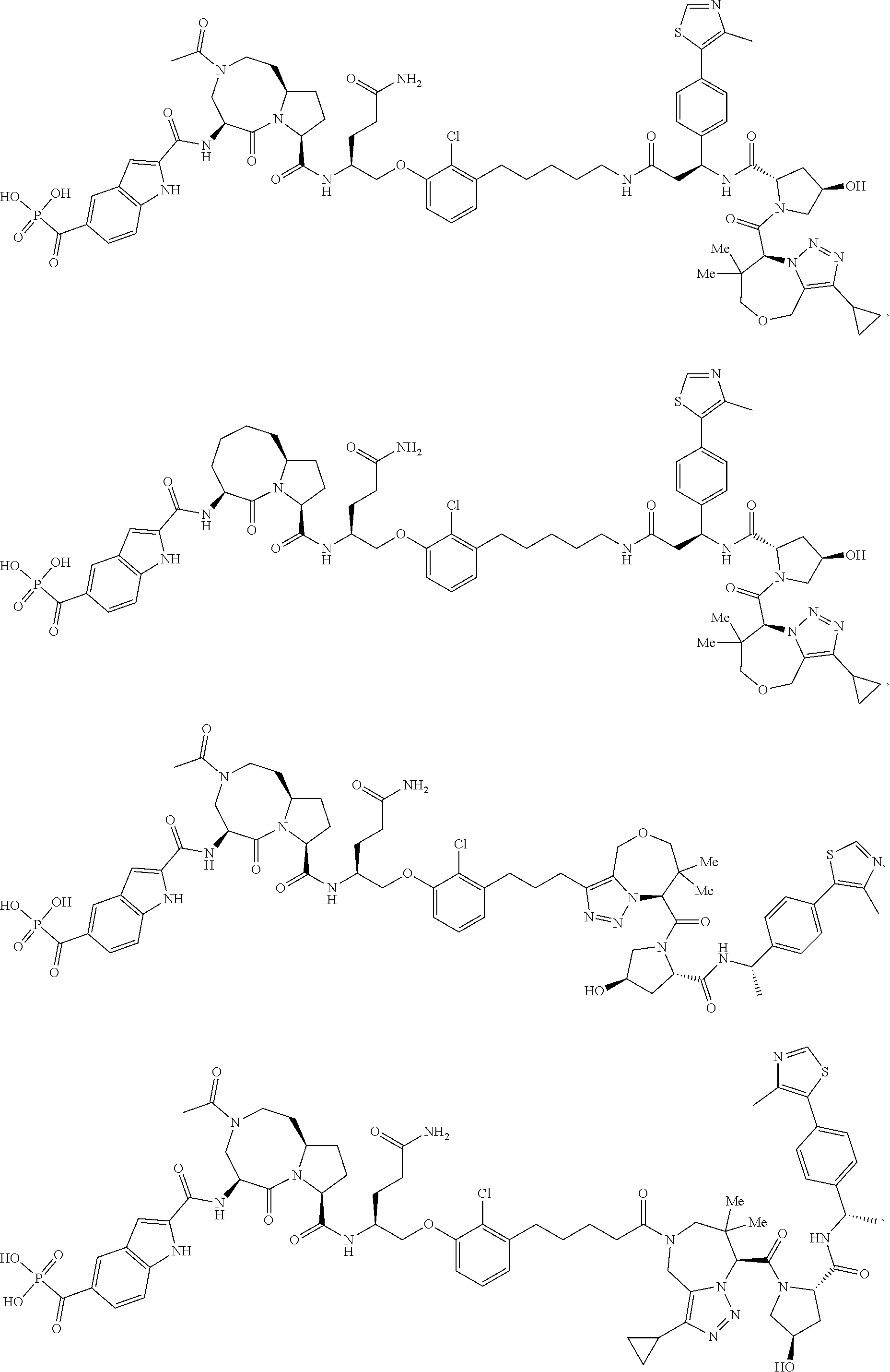 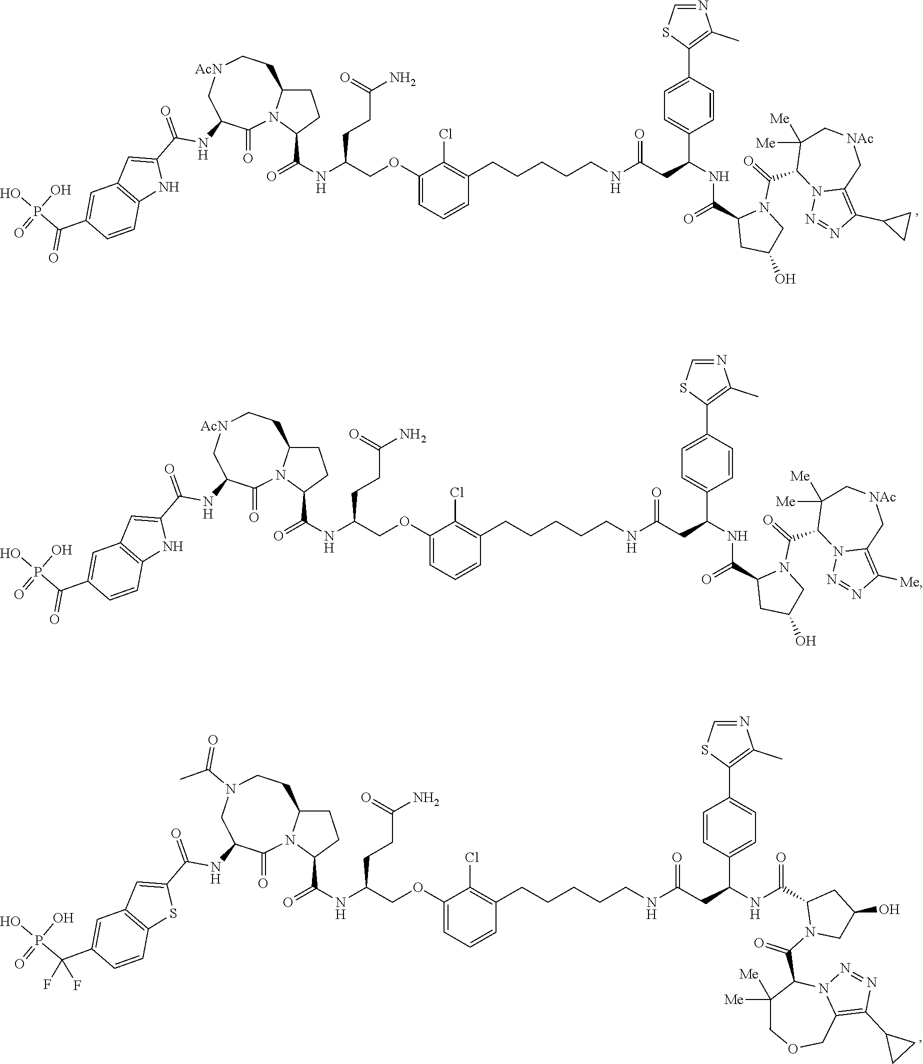 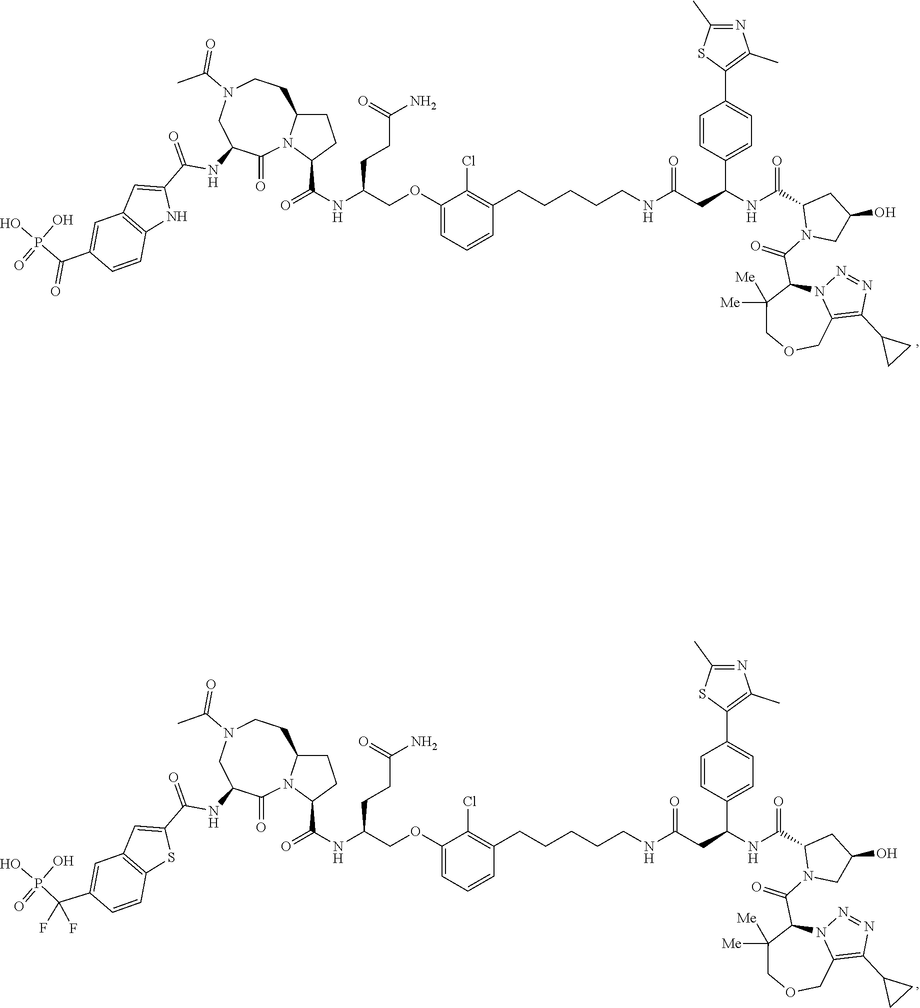 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](NC(=O)c2cc3cc(C(=O)P(=O)(O)O)ccc3[nH]2)C1 ; CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5sc(C)nc5C)cc4)c3Cl)N2C(=O)[C@@H](NC(=O)c2cc3cc(C(F)(F)P(=O)(O)O)ccc3s2)C1] 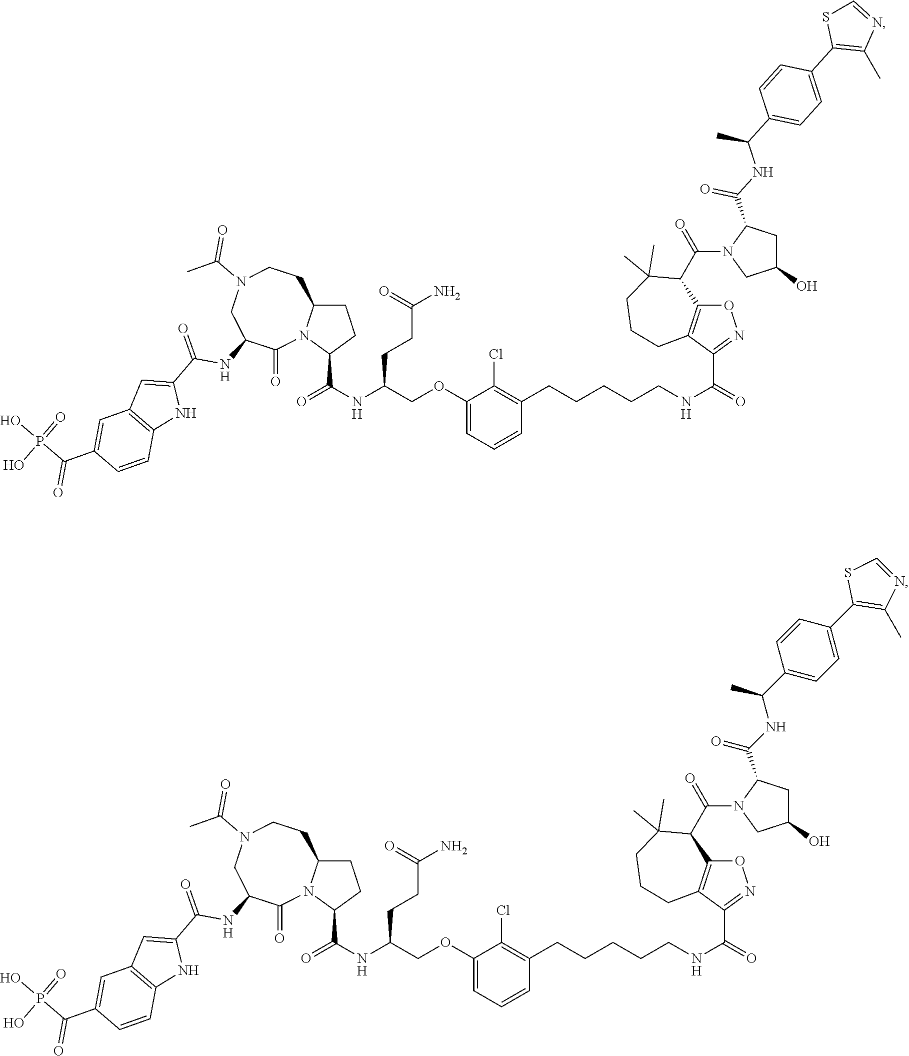 [SMILES: CC(=O)N1CC[C@H]2CC[C@@H](C(=O)N[C@@H](CCC(N)=O)COc3cccc(CCCCCNC(=O)c4noc5c4CCCC(C)(C)[C@@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1 ; CC(=O)N1CC[C@H]2CC[C@@H](C(=O)N[C@@H](CCC(N)=O)COc3cccc(CCCCCNC(=O)c4noc5c4CCCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1] 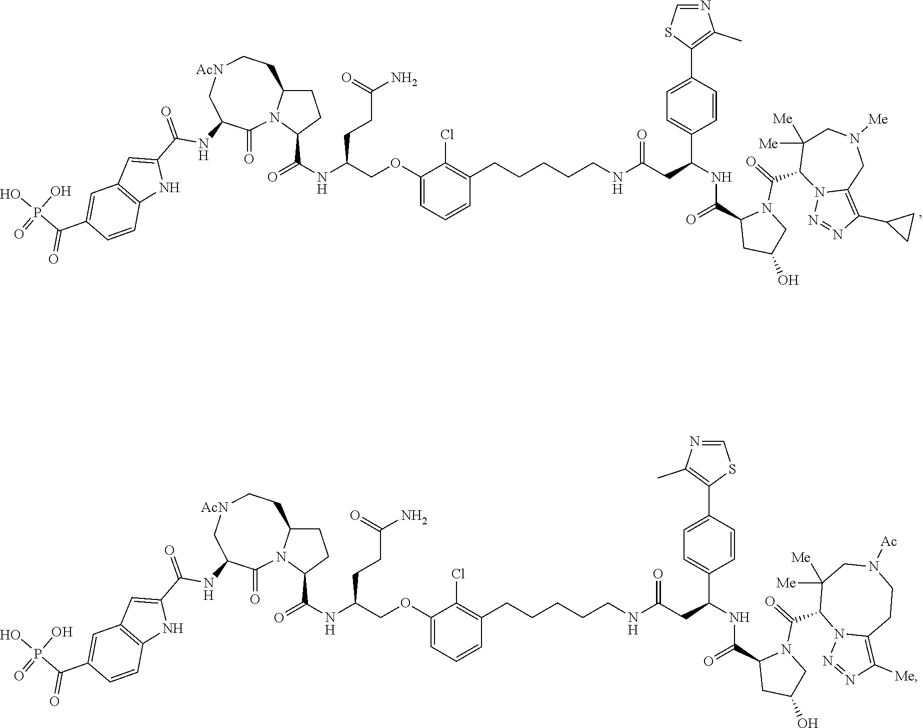 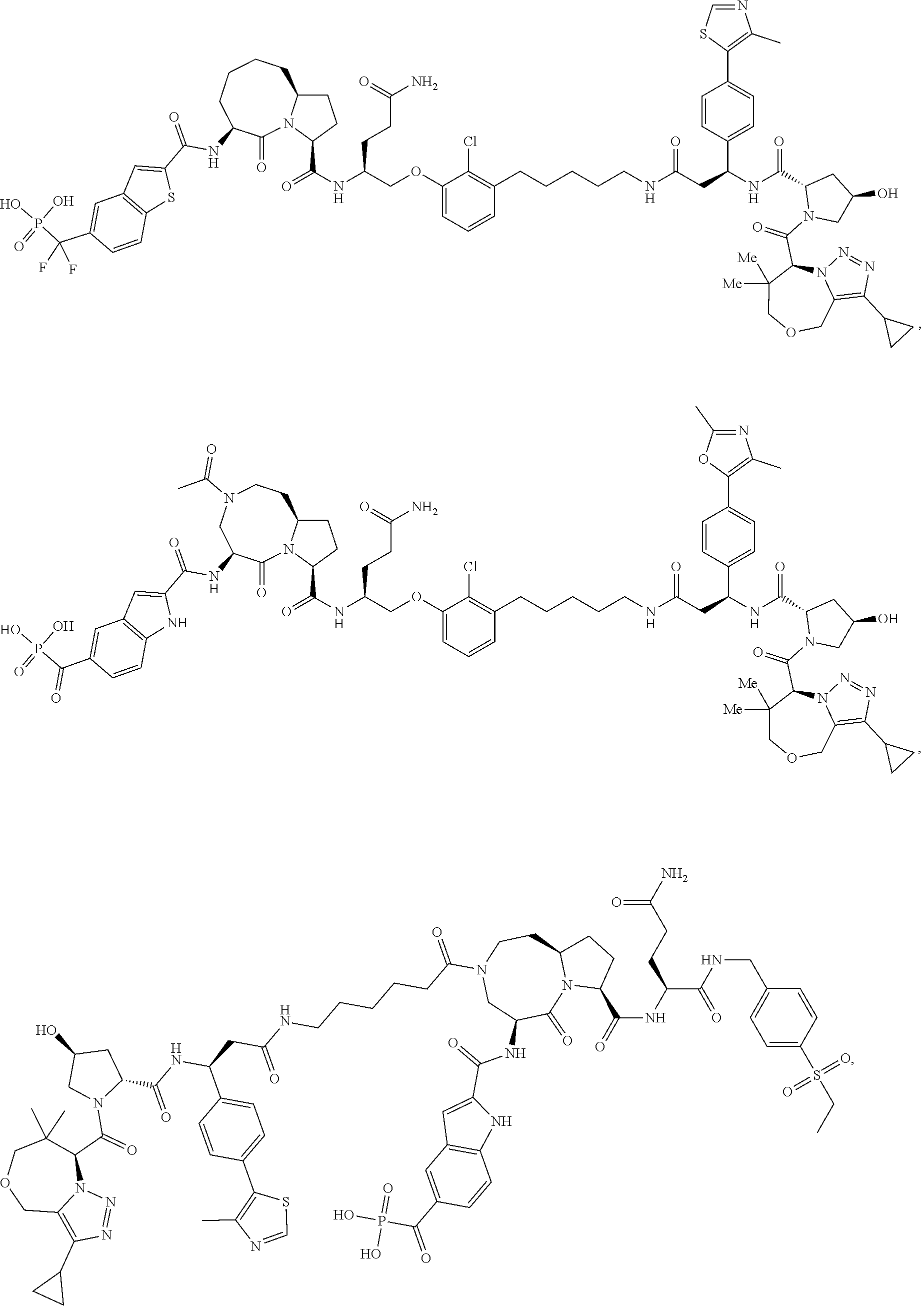 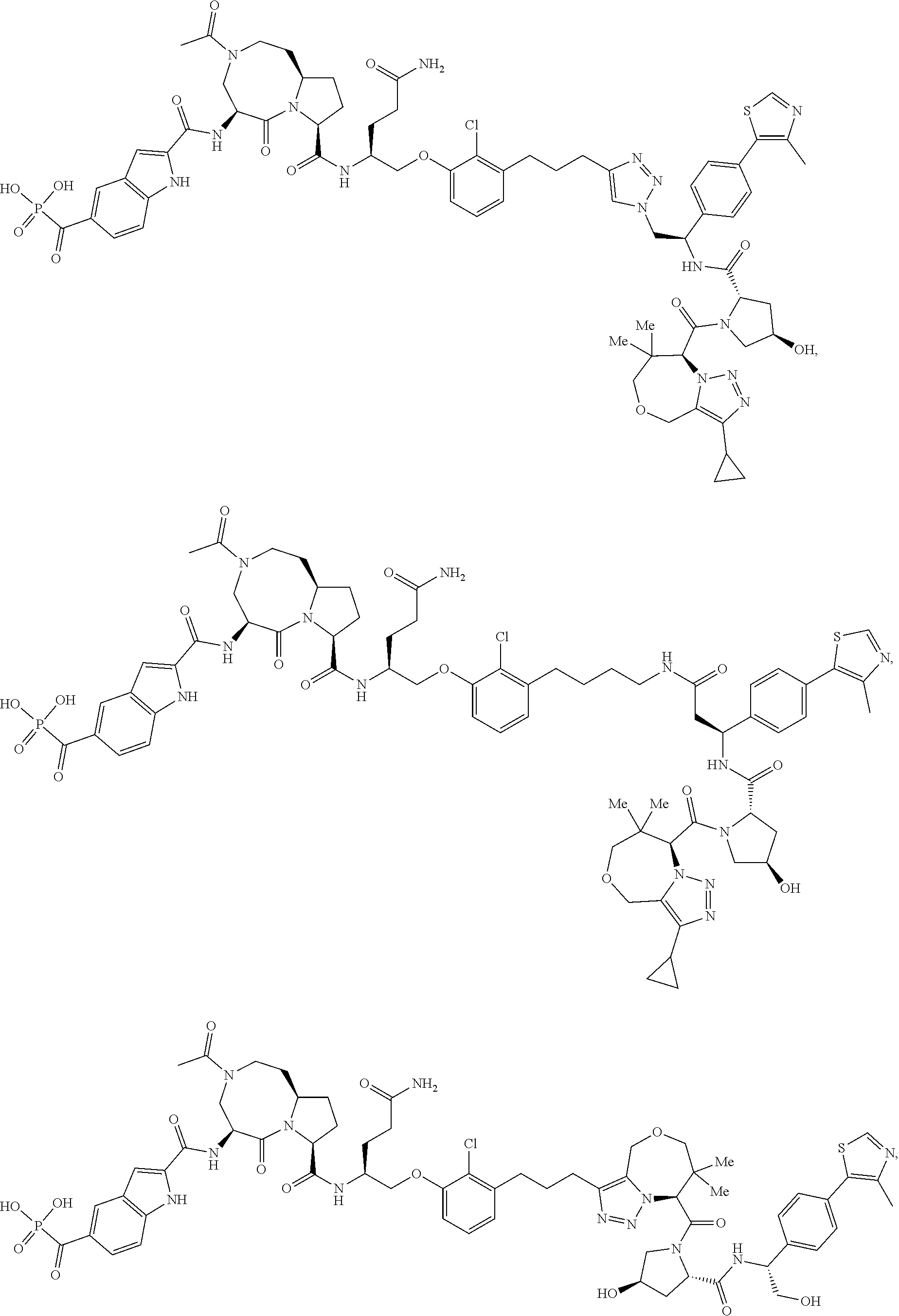 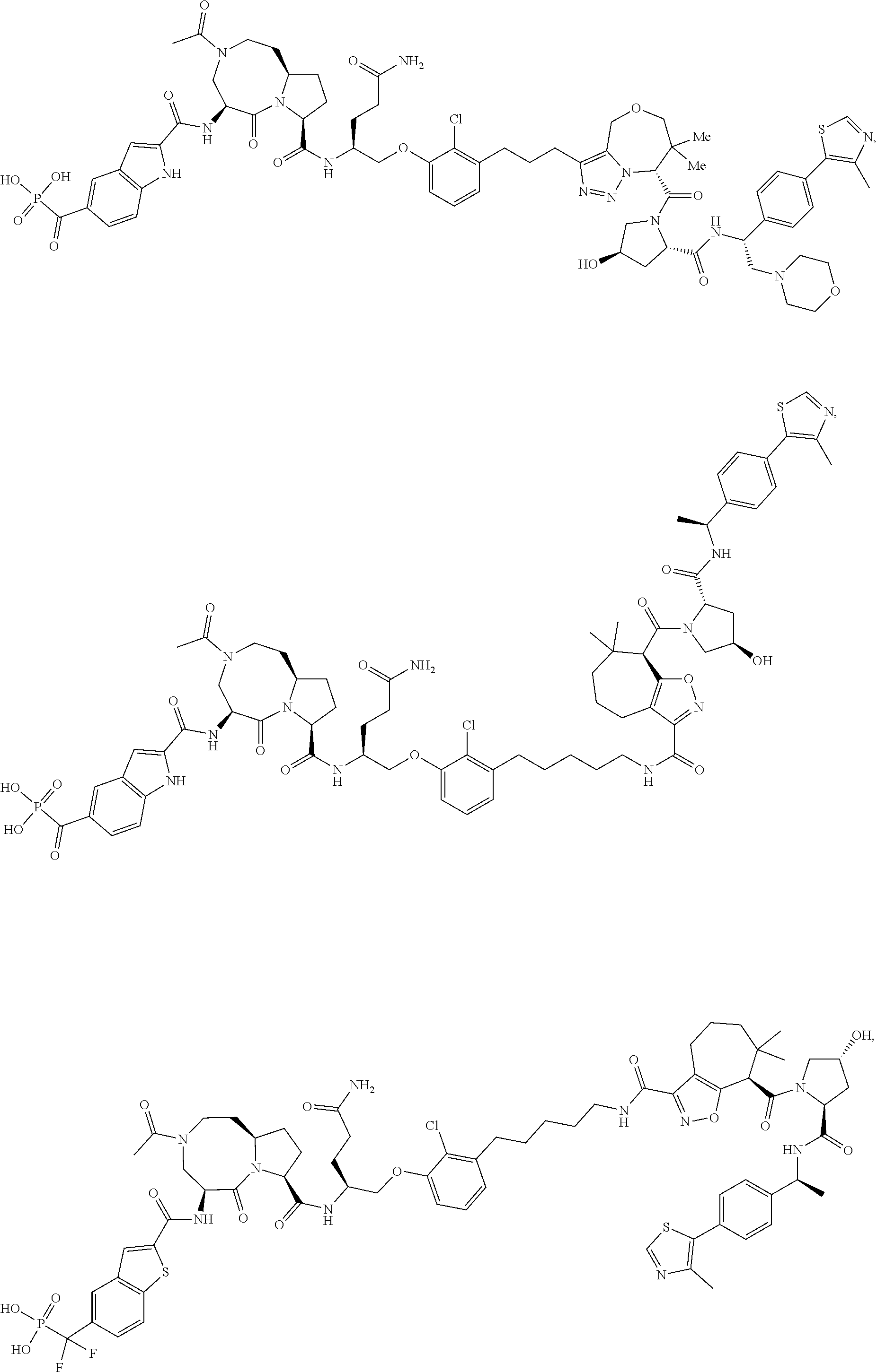 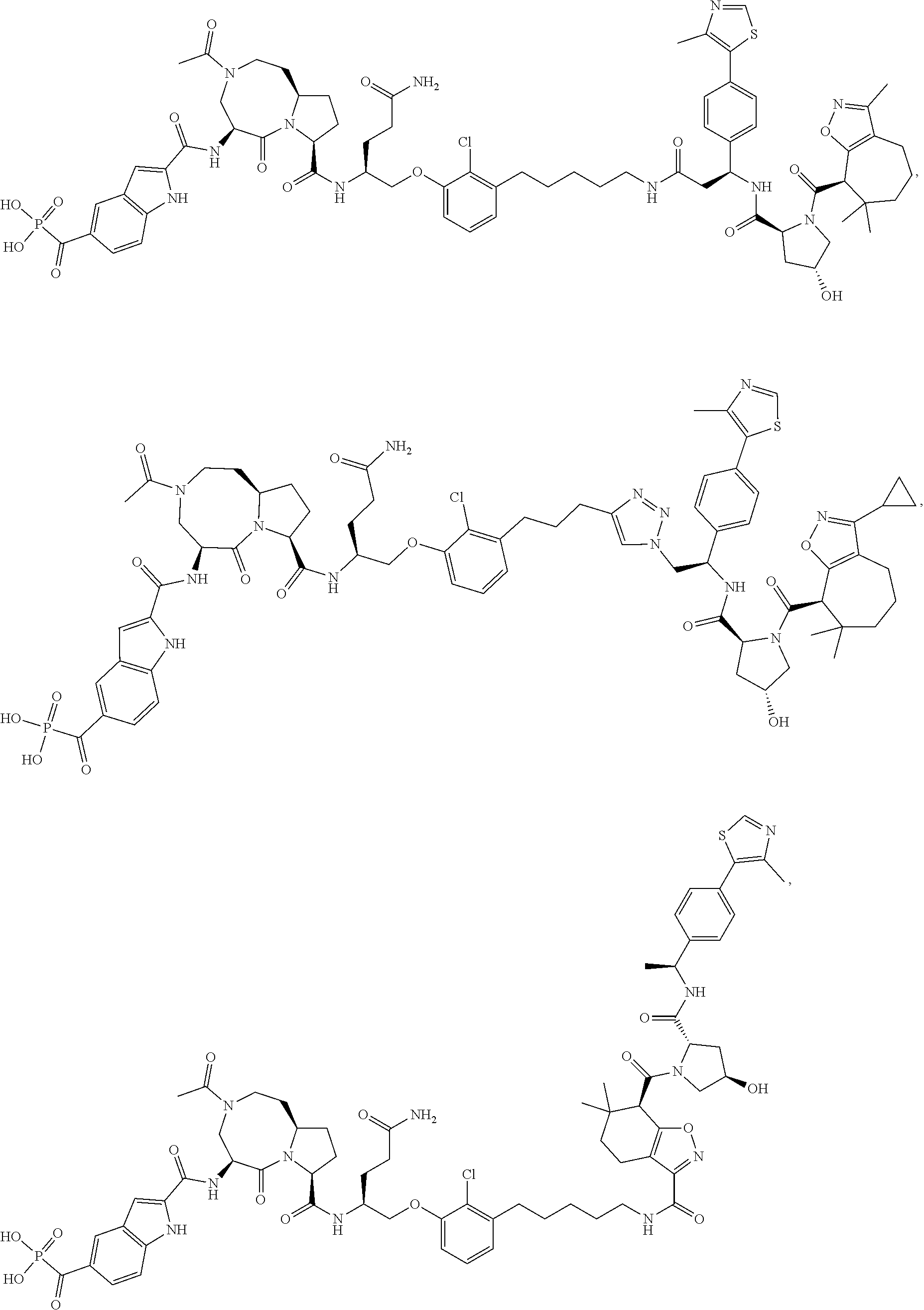 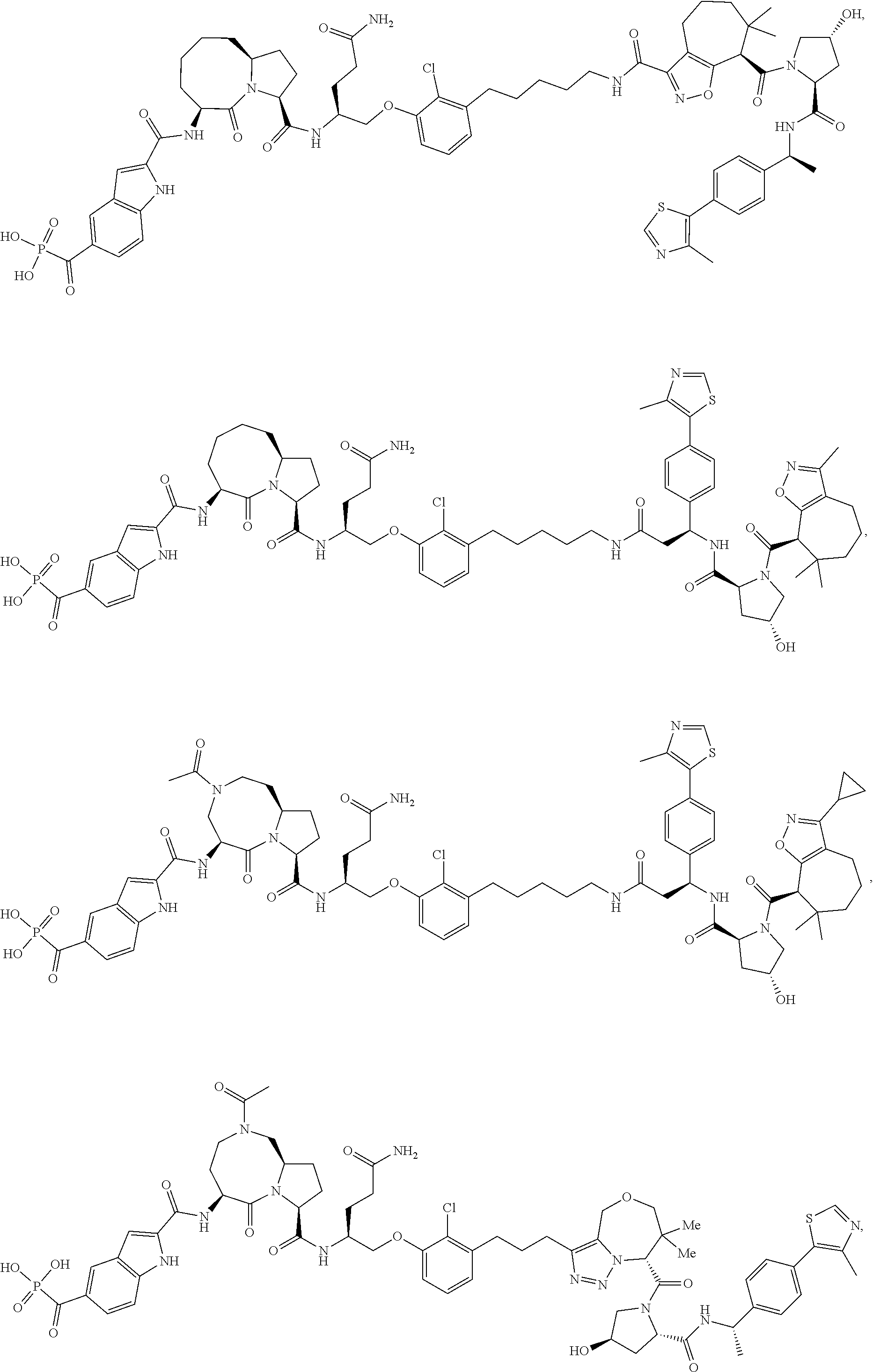 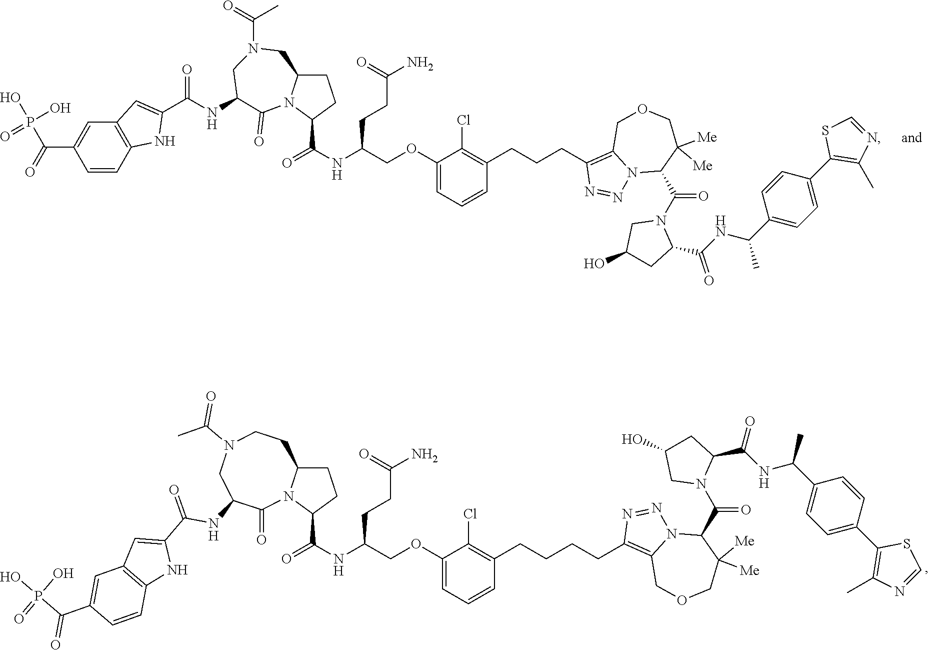 [SMILES: CC(=O)N1C[C@H]2CC[C@@H](C(=O)N[C@@H](CCC(N)=O)COc3cccc(CCCc4nnn5c4COCC(C)(C)[C@@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1 ; "and" ; CC(=O)N1CC[C@H]2CC[C@@H](C(=O)N[C@@H](CCC(N)=O)COc3cccc(CCCCc4nnn5c4COCC(C)(C)[C@@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1]
or a pharmaceutically acceptable salt thereof.

130. The conjugate of claim 129, or a pharmaceutically acceptable salt thereof, that is:

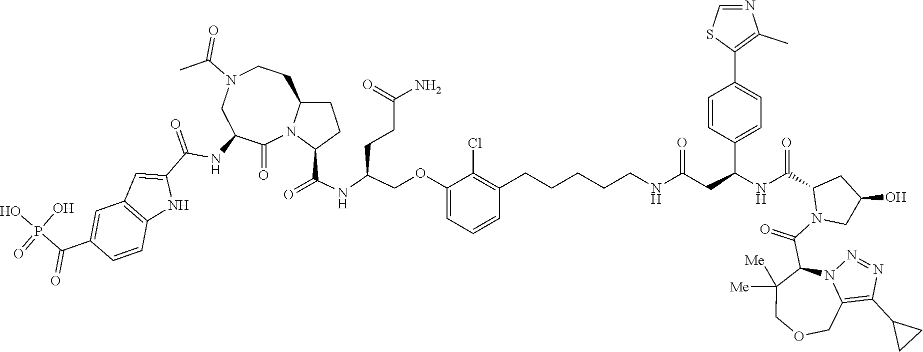 [SMILES: CC(=O)N1CC[C@H]2CC[C@@H](C(=O)N[C@@H](CCC(N)=O)COc3cccc(CCCCCNC(=O)C[C@H](NC(=O)[C@@H]4C[C@@H](O)CN4C(=O)[C@H]4n5nnc(C6CC6)c5COCC4(C)C)c4ccc(-c5scnc5C)cc4)c3Cl)N2C(=O)[C@@H](NC(=O)c2cc3cc(C(=O)P(=O)(O)O)ccc3[nH]2)C1]

131. The conjugate of claim 129, or a pharmaceutically acceptable salt thereof, that is:

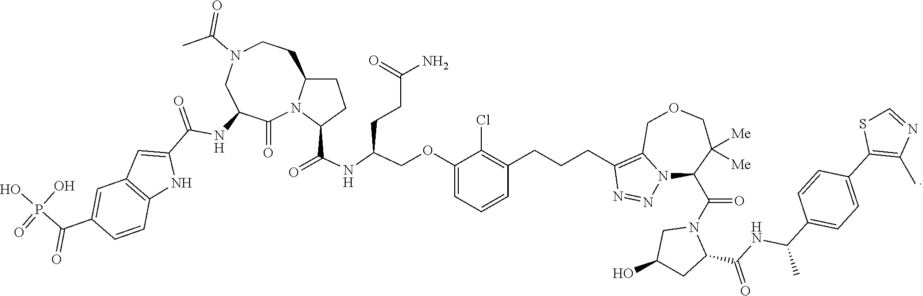 [SMILES: CC(=O)N1CC[C@H]2CC[C@@H](C(=O)N[C@@H](CCC(N)=O)COc3cccc(CCCc4nnn5c4COCC(C)(C)[C@H]5C(=O)N4C[C@H](O)C[C@H]4C(=O)N[C@@H](C)c4ccc(-c5scnc5C)cc4)c3Cl)N2C(=O)[C@@H](NC(=O)c2cc3cc(C(=O)P(=O)(O)O)ccc3[nH]2)C1]

132. The conjugate of claim 129, or a pharmaceutically acceptable salt thereof, that is:

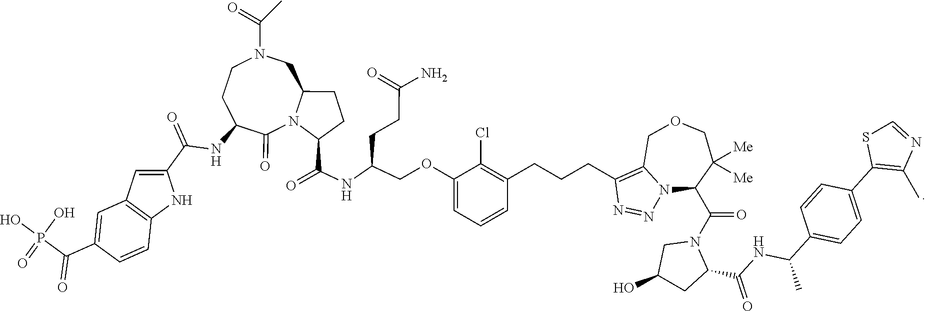 [SMILES: CC(=O)N1CC[C@H](NC(=O)c2cc3cc(C(=O)P(=O)(O)O)ccc3[nH]2)C(=O)N2[C@H](CC[C@H]2C(=O)N[C@@H](CCC(N)=O)COc2cccc(CCCc3nnn4c3COCC(C)(C)[C@H]4C(=O)N3C[C@H](O)C[C@H]3C(=O)N[C@@H](C)c3ccc(-c4scnc4C)cc3)c2Cl)C1]

133. A pharmaceutical composition comprising the conjugate of claim 113, or a pharmaceutically acceptable salt thereof, and a pharmaceutically acceptable excipient.

134. A method of treating a STAT3-mediated disease or disorder comprising administering to a patient in need thereof a conjugate of claim 113, wherein the STAT3-mediated disease or disorder is cancer, an autoimmune disease, an inflammatory disorder, a neurodegenerative disease, a viral disease, a hereditary disorder, a hormone-related disease, a metabolic disorder, a condition associated with organ transplantation, an immunodeficiency disorder, a destructive bone disorder, a proliferative disorder, an infectious disease, a condition associated with cell death, thrombin-induced platelet aggregation, liver disease, a pathologic immune condition involving T cell activation, a cardiovascular disorder, or a CNS disorder.

135. A compound of Formula I: 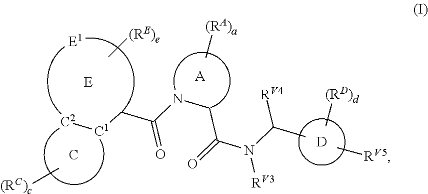
or a pharmaceutically acceptable salt, solvate, or stereoisomer thereof, wherein:
Ring A is 5- to 7-membered heterocycle;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
a is an integer selected from 0 to 10, as valency permits;
Ring C is C₆ aryl or 5- to 6-membered heteroaryl;
C′ and C² are independently C or N;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
c is an integer selected from 0 to 5, as valency permits;
Ring D is C₆ aryl or 5- to 6-membered heteroaryl;
each R^{D} is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 4;
Ring E is C₅₋₁₂ carbocycle or 5- to 12-membered heterocycle, wherein the carbocycle or heterocycle is optionally substituted with one or more R^{E};
E¹ is C(R^{E1′})₂, NR^{E1″}, O, or S;
each R^{E1′} is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u};
R^{E1″} is an amino-protecting group, hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
each R^{E} is independently oxo, hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; or
two geminal R^{E}, together with the carbon atom to which they are bonded, form C₃₋₆ carbocycle or 3- to 6-membered heterocycle, wherein the carbocycle or heterocycle is optionally substituted with one or more R^{u};
e is an integer selected from 0 to 6;
R^{V3} is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}; wherein the alkyl, carbocyclyl, or heterocyclyl is optionally substituted with one or more R^{u};
R^{V4} is hydrogen, C₁₋₆ alkyl, or C₁₋₈ heteroalkyl, wherein the alkyl or heteroalkyl is optionally substituted with one or more R^{u}; and
R^{V5} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u},
wherein:
each R^{u} is independently oxo, halogen, —N₃, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{c}S(═O)₂R^{a}, —NR^{c}S(═O)R^{a}, —NR^{c}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or
two R^{u}, together with the one or more intervening atoms, form C₃₋₁₂ carbocyclyl, 3- to 12-membered heterocyclyl, C₆₋₁₀ aryl, or 5- to 10-membered heteroaryl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, R^{b}, R^{c}, and R^{d} is independently and optionally substituted with one or more R^{z}; and
each R^{z} is independently oxo, halogen, —CN, —NO₂, —OH, —NH₂, C₁₋₆ alkyl, C₁₋₆ alkoxy, CI-6 alkylamino, C₂₋₆ alkenyl, C₂₋₆ alkynyl, C₃₋₆ carbocyclyl, or 3- to 6-membered heterocyclyl.

136. The compound of claim 136, wherein the compounds is selected from the group consisting of: 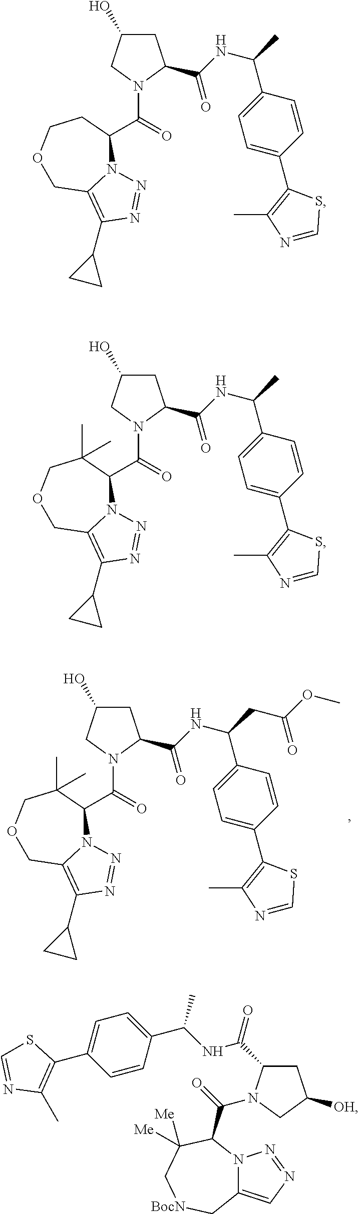 [SMILES: Cc1ncsc1-c1ccc([C@H](C)NC(=O)[C@@H]2C[C@@H](O)CN2C(=O)[C@@H]2CCOCc3c(C4CC4)nnn32)cc1 ; Cc1ncsc1-c1ccc([C@H](C)NC(=O)[C@@H]2C[C@@H](O)CN2C(=O)[C@H]2n3nnc(C4CC4)c3COCC2(C)C)cc1 ; COC(=O)C[C@H](NC(=O)[C@@H]1C[C@@H](O)CN1C(=O)[C@H]1n2nnc(C3CC3)c2COCC1(C)C)c1ccc(-c2scnc2C)cc1 ; Cc1ncsc1-c1ccc([C@H](C)NC(=O)[C@@H]2C[C@@H](O)CN2C(=O)[C@H]2n3nncc3CN(C(=O)OC(C)(C)C)CC2(C)C)cc1] 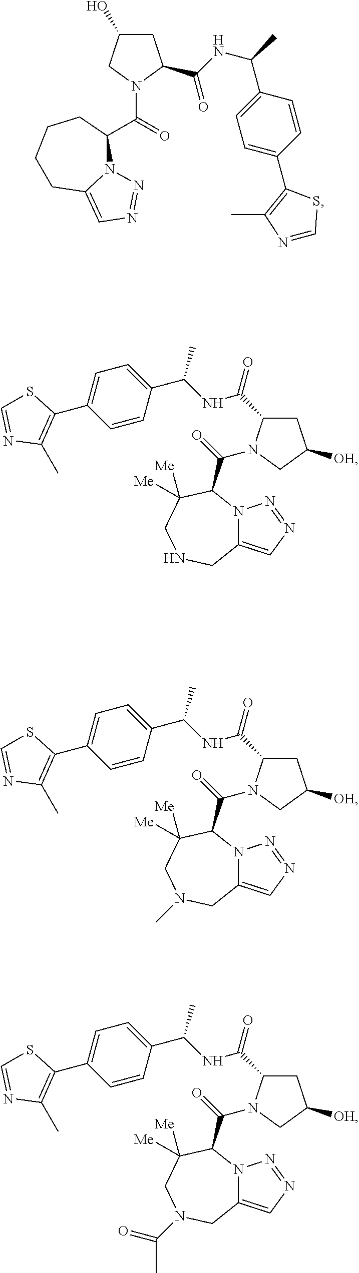 [SMILES: Cc1ncsc1-c1ccc([C@H](C)NC(=O)[C@@H]2C[C@@H](O)CN2C(=O)[C@@H]2CCCCc3cnnn32)cc1 ; Cc1ncsc1-c1ccc([C@H](C)NC(=O)[C@@H]2C[C@@H](O)CN2C(=O)[C@H]2n3nncc3CNCC2(C)C)cc1 ; Cc1ncsc1-c1ccc([C@H](C)NC(=O)[C@@H]2C[C@@H](O)CN2C(=O)[C@H]2n3nncc3CN(C)CC2(C)C)cc1 ; CC(=O)N1Cc2cnnn2[C@H](C(=O)N2C[C@H](O)C[C@H]2C(=O)N[C@@H](C)c2ccc(-c3scnc3C)cc2)C(C)(C)C1] 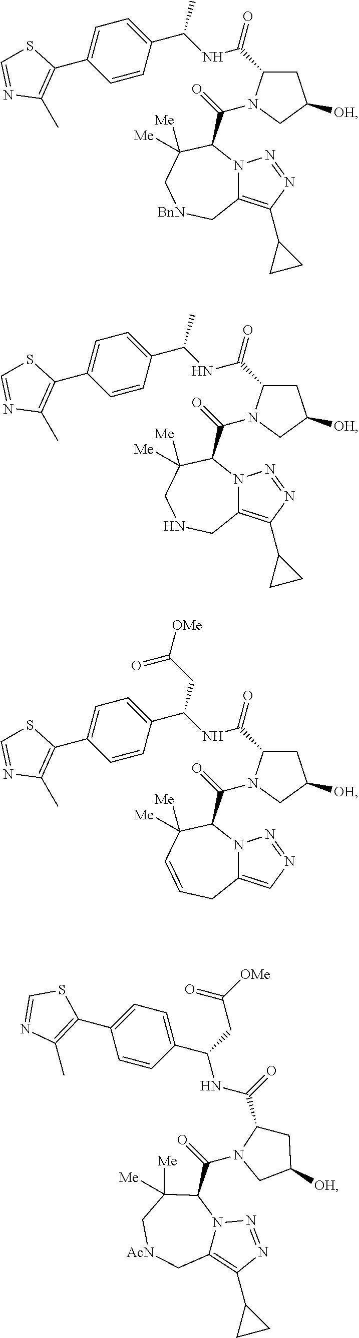 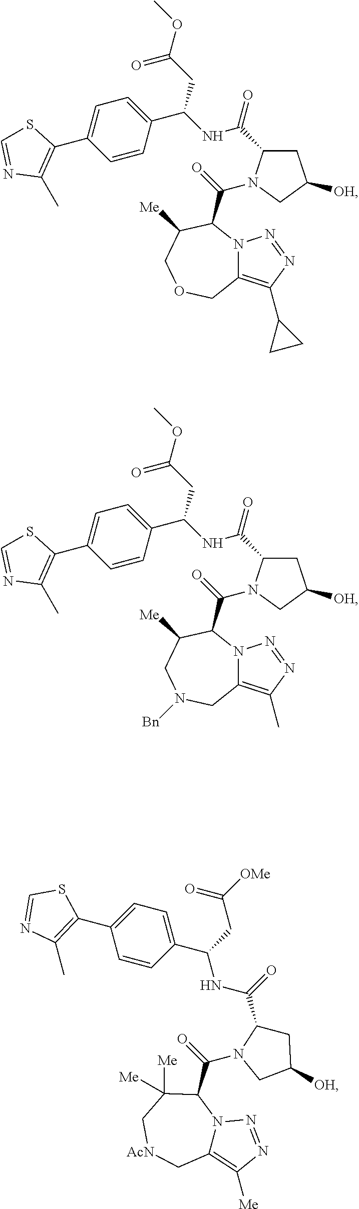 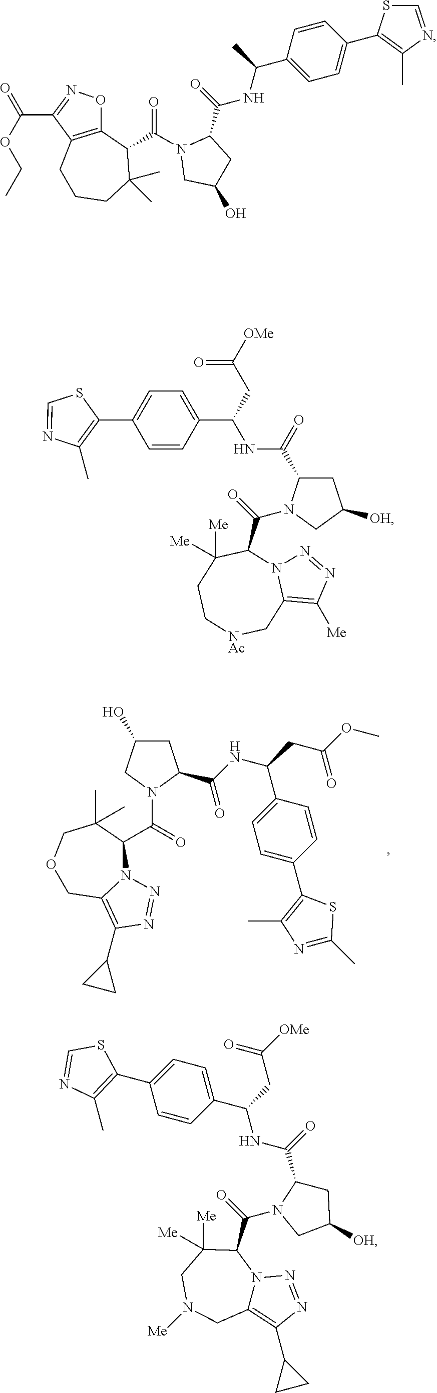 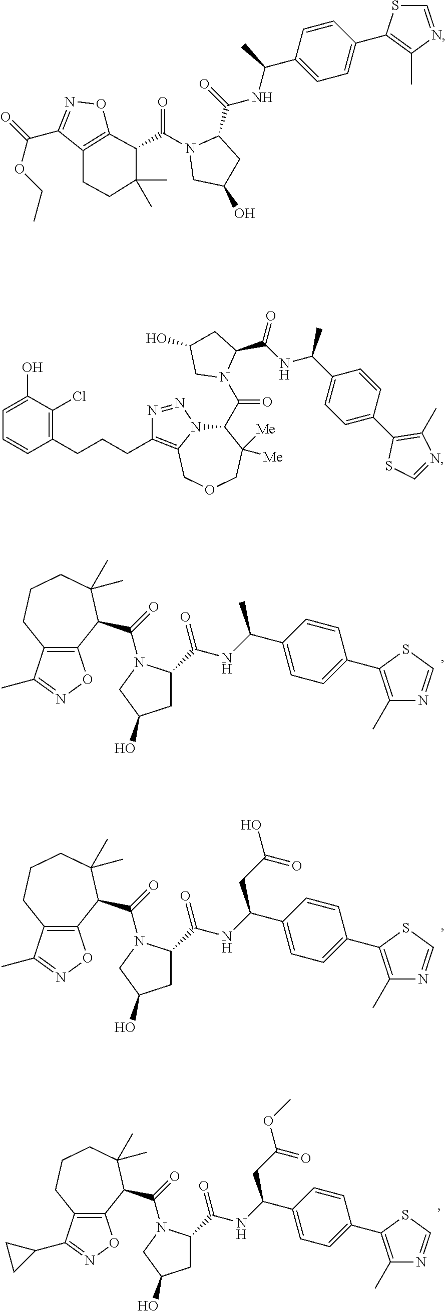 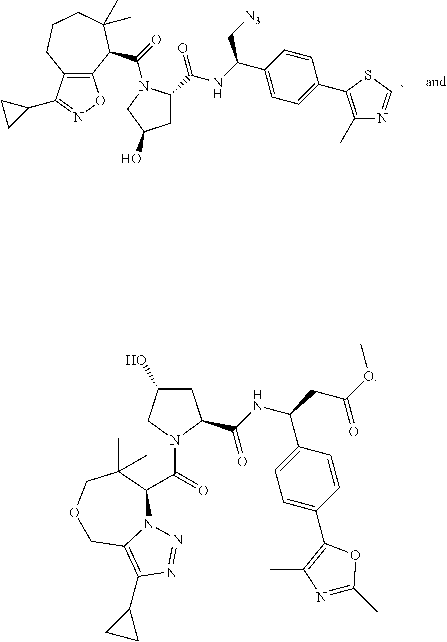 [SMILES: Cc1ncsc1-c1ccc([C@H](CN=[N+]=[N-])NC(=O)[C@@H]2C[C@@H](O)CN2C(=O)[C@H]2c3onc(C4CC4)c3CCCC2(C)C)cc1 ; COC(=O)C[C@H](NC(=O)[C@@H]1C[C@@H](O)CN1C(=O)[C@H]1n2nnc(C3CC3)c2COCC1(C)C)c1ccc(-c2oc(C)nc2C)cc1]
